(19) United States
(12) Patent Application Publication
(10) Pub. No.: US 2023/0392133 A1
(43) Pub. Date: Dec. 7, 2023

(54) Compositions and Methods Relating to Alzheimer's Disease

(71) Applicant: Duke University, Durham, NC (US)

(72) Inventors: Ornit Chiba-Falek, Durham, NC (US); Boris Kantor, Durham, NC (US)

(73) Assignee: Duke University, Durham, NC (US)

(21) Appl. No.: 18/033,166

(22) PCT Filed: Oct. 12, 2021
(86) PCT No.: PCT/US21/54475

§ 371 Date: Apr. 21, 2023

Related U.S. Application Data
(60) Provisional application No. 63/132,286, filed on Dec. 30, 2020.
(60) Provisional application No. 63/104,343, filed on Oct. 22, 2020.

(51) Int. Cl. C12N 9/22; C12N 15/86; A61P 25/28

(52) U.S. Cl. CPC C12N 9/22; C12N 15/86; A61P 25/28; C12N 2310/20; C12N 2740/15043

(57) ABSTRACT

Disclosed herein are methods of administering precision gene therapy, treating and/or preventing Alzheimer' disease progression, and reducing expression of APOE and APOE e4. Disclosed herein are isolated nucleic acid molecules, viral vectors, lentiviral vectors, pharmaceutical formulations, host cells, guide RNAs and plasmids for use in the disclosed methods.

### I. CROSS-REFERENCE TO RELATED APPLICATIONS

This application claims the benefit of priority to U.S. Provisional Patent Application No. 63/104,343 filed 22 Oct. 2020 and U.S. Provisional Patent Application No. 63/132,286 filed 30 Dec. 2020, both of which are incorporated by reference herein in their entirety.

### II. REFERENCE TO THE SEQUENCE LISTING

The Sequence Listing submitted 12 Oct. 2021 as a text file named “20_940_WO_Sequence_Listing”, created on 12 Oct. 2021 and having a size of 581 kilobytes is hereby incorporated by reference pursuant to 37 C.F.R. § 1.52(e)(5).

### III. BACKGROUND

Alzheimer's disease (AD) is sixth leading cause of death in the US and the most common cause of dementia in aging. With a rapidly growing aging population the number of AD cases is growing fast and projected to rise drastically over the next three decades. Today, more than 5 million people are living with AD in the United States alone, and by 2050, this number is projected to reach 14 million. Therefore, AD poses a huge economic burden on society placing overwhelming strain on the healthcare system. In 2020, the cost of AD to the US was $301 billion, including $206 billion in Medicare and Medicaid payments, while the caregivers provided $244 billions worth of care (Alzheimer's Association, Alzheimer's Impact Movement: Factsheet 2020). These trends will only worsen with time because there are no therapies to halt or prevent AD (i.e., projected to cost more than $1.1 trillion annually by 2050). Despite all the research effort, money and commitment, clinical trials to identify disease modifying therapies (DMT) for AD have repeatedly failed. To date, there is no cure and no DMT for AD and there are no methods to delay the onset and/or progression of the disease. Most available treatments are palliative and aimed at relieving symptom (Sharma K. (2019) Mol Med Rep. 20:1479-1487; Olivares D, et al. (2012) Curr Alzheimer Res. 9:746-758).

Late onset AD (LOAD) is a heterogenous disease with various genetic etiologies (Lo M T, et al. (2019). Neurobiol Aging. 84:243 e1-243.e9; Nacmias B, et al. (2018) J Alzheimers Dis. 62:903-911). A major reason for the failure to identify an effective treatment is likely the inaccurate consideration of LOAD as a homogeneous disease. In this respect, increasing evidence demonstrate the heterogeneity in the underlying pathophysiologic processes of LOAD and show variability in the genetic risk and molecular profiles amongst AD patients (Reitz C. (2016) Ann Transl Med. 4:107; Chiba-Falek O, et al. (2017) Expert Rev Precis Med Drug Dev. 2:47-55). Thus, AD remains an unmet medical need underscoring the urgent need for a paradigm shift in AD clinical research.

Accordingly, any advancement in LOAD therapy will require the development and validation of new therapeutic targets including those tailored to sub-groups of patients with specific risk factors. Thus, to date many investigators and funding bodies recognize the need to shift the focus to new potential culprits including candidate gene-targets such as LOAD risk genes and rare mutations (Guerreiro R, et al. (2013) Neurobiol Aging. 34:2890 e1-5). Consistently, recently, alternative targets such as APOE have emerged as potential promising targets for LOAD treatment (Huynh T V, et al. (2017) Neuron. 96:1013-1023 e4; Brody D L, et al. (2008) Annu Rev Neurosci 31:175-193; Kim J, et al. (2012) J Exp Med. 209:2149-2156).

Accordingly, there is a need for a disease modifying therapy for Alzheimer's disease, particularly LOAD.

### IV. BRIEF DESCRIPTION OF THE FIGURES

FIG. 1 shows the effect of APOE genotypes on APOE-mRNA levels. FIG. 1A shows a schematic model describing the mechanisms that lead to increased ApoE activity and by that mediate the pathogenic effect of APOE e4 and APOE e3 (differ in amino acid at position 112 Arg and Cys, respectively) on LOAD. FIG. 1B shows a diagram of the different technologies to target ApoE, including antisense oligonucleotide (ASO), monoclonal antibody (mAbs), and CRISPR/Cas9 gene editing technologies. The fold levels of human APOE mRNA were assayed using qRT-PCR in temporal tissues (FIG. 1C) and in occipital tissues (FIG. 1D). FIG. 1E shows the level of human APOE-mRNA in whole brain tissues from humanized mice assayed by qRT-PCR.

FIG. 2 shows a schematic representation of APOE gene. The APOE gene is located at chromosome 19q13.2. The SNP rs429358 changes amino acid in position 112 and defines APOE e4 allele. The SNP rs7412 changes amino acid in position 158 and defines the APOE e2 allele. The CpG island in exon 4 is highlighted. DMRI and DMR2 regions are defined by two CGIs, which are marked in a yellow box. Exons 1-4 are designated in boxes. The translated exons are highlighted in dark blue. 5′-UTR and 3′-UTR of the gene are highlighted in light blue.

FIG. 3 shows the DNA-methylation profile of the APOE LD region in FANS-sorted neuronal and non-neuronal nuclei. FIG. 3A shows a map of MethylEPIC array probes in the chr19: 45,393,000-45,424,000; hg19. The red circles represent probes with >0.5 methylation levels while the blue circles represent probes with <0.5 methylation levels. The APOE promoter region is hypomethylated and is an excellent target region for enhancement of DNA-methylation. FIG. 3B shows that probes showed significant differences in methylation levels between NeuN⁺ (n=16), NeuN⁻ sorted nuclei (n=16), or LOAD (n=8) vs. Normal (n=8). Solid bars represent neuronal population while the hatched bars represent the non-neuronal population. The accompanying table summarizes the p-values for each of the significant probes.

FIG. 4 show the structure of human APOE gene and the design of spCas9 gRNAs to target the promoter region of the gene. FIG. 4 shows the genomic organization of the gene including the two SNPs in exon 4 and the gRNA targeting of the promoter region of the gene. The 5′-UTR and 3′-UTR of the gene are also shown.

FIG. 5 shows the schematic representation of lentiviral vector system carrying DNMT3A to target the promoter and exon 4 regions of APOE gene. The 5′-LTR and the 3′-LTR represent long terminal repeats. Phi represents the packaging signal of the vector. RRE represents the rev responsive element responsible for binding REV protein of the virus. The Sp1 responsive element inclusion (Ortiniski et al. (2017); Kantor et al. (2018)) demonstrated high production yield. The hU6 promoter drives expression of the gRNA and the EFS-NC promoter drives the expression of dCAS9 (to target promoter of APOE) or dVRER to target SNP (112) at the exon 4 region. The p2A signal separates the effector molecule from GFP/Puro reporters. WPRE is the Woodchuck Hepatitis Virus (WHP) Post-Transcriptional Regulatory Element (WPRE), which is a DNA sequence that when transcribed creates a tertiary structure enhancing expression. The arrow pointing to the promoter region highlights the binding of the dCas9-DNMT3A-gRNA to the promoter region or the SNP region that results in the DNA methylation (red lollipops) and downregulation of gene expression (represented with the red cross sign).

FIG. 6 shows the targeting of the promoter region of APOE with gRNA-dCas9-DNMT3A lentiviral vector system. Human hepatocytes HEPG2 cell (having APOEe3/3 genotype) were stably transduced with lentiviral vector carrying 4 different gRNA paired with dCas9-DNMT3A or dCAS9-DNMT3A null vectors. In FIG. 6, the levels of the mRNA and protein downregulation were compared to untransduced naïve HEPG2 cells. The vectors delivering the active version of DNMT3A represented in white bars while the null mutants are shown in black bars. The experiments were repeated three time and the SD bars are highlighted. FIG. 6A shows the levels of RNA knockdown following the transduction with a lentiviral vector as assessed by real-time PCR. gRNA1 showed the most robust reduction in APOE-mRNA. FIG. 6B shows the levels of protein knockdown following the transduction with a lentiviral vector as assessed by western blot. The effects on the protein levels were comparable with the effects on the mRNA shown in FIG. 6A, demonstrating the most robust decrease in protein levels was driven by gRNA1. In FIG. 6, gRNA1 was gacagggggagccctataat (SEQ ID NO:25), gRNA3 was actgggatgtaagccatagc (SEQ ID NO:27), and gRNA4 was gttggagcttagaatgtgaa (SEQ ID NO:28).

FIG. 7 shows the structure of humanAPOE gene and VRER gRNAs design relative to the spCas9 gRNAs positions targeting the promoter region of the gene. Genomic organization of the gene outlined in the lower panel highlighting the 2 SNPs within exon 4. gRNA targeting promoter region of the gene are outlined. spCas9 gRNAs (in green) and VRER gRNAs (in yellow) positions. The 5′-UTR and the 3′-UTR of the gene are indicated in boxes. Structure of a human APOE gene and VRER vs spCas9 gRNAs locations are shown.

FIG. 8A-FIG. 8B show the validation of VRER system using GFP-reporter cells. A GFP-reporter 293T cell line was created by stable transduction using lentiviral vector. GFP was subjected to site-directed mutagenesis to change the PAM motif for VRER enzyme NGCG to GGG, which is recognized by SpCas9. The cells identified as 1003GFP⁻ are generated to include this modification. The target cells were transduced with SpCas9-gRNA-to-GFP vector VRER-gRNA-to-GFP vector to assess the specificity and efficacy of the corresponding enzymes. The gRNA sequence selected for targeting is highlighted. The cells identified as 201A GFP cells (FIG. 8A) contained the “naïve” GFP sequence, while 1003 GFP cells (FIG. 8B) were introduced with point-substitution (as above) without changing amino acid residues. A score of 5+ highlights the high efficiency of the GFP cleavage, while a score of 5− highlights incapacity of the enzyme to digest DNA. The specificity of VRER was found to be comparable to that of Cas9 while the efficacy was demonstrated to be significantly lower.

FIG. 9 shows the effect of targeting the promoter region of APOE with a gRNA-dVRER-DNMT3A lentiviral vector system. Human hepatocytes HEPG2 cells were stably transduced with lentiviral vector carrying 4 different gRNA paired with dVRER-DNMT3A or dVRER-DNMT3A null vectors. FIG. 9 shows the level of RNA knockdown following the transduction using real-time PCR. The levels of the mRNA downregulation was compared to untransduced, naïve HEPG2 cells. The vectors delivering the active version of DNMT3A are represented in white bars while the null mutants are represented in black bars. The experiments were repeated three times and the SD bars are highlighted.

FIG. 10A-FIG. 10F shows the differentiation and characterization of hiPSC-derived neurons. FIG. 10A shows the timeline for neuronal differentiation. FIG. 10B shows representative immunocytochemistry of hiPSC-derived neurons. FIG. 10C shows the FACS-analysis showing co-expression of TUBB3 and VachT (36.4%) while FIG. 10D shows the absence of GFAP signal. FIG. 10E shows the relative expression levels of the neuronal-specific markers (TUBB3 and CHAT) and the astrocyte specific marker (GFAP). FIG. 10F shows APOE-mRNA expression in isogenic APOE 3/3 and 4/4 hiPSC-derived neurons. APOE-mRNA 3/3>4/4 consistent with the observation in human brain, which demonstrated the suitability of the system for drug discovery.

FIG. 11A-FIG. 11C show expression levels and immunohistochemical staining of isogenic APOE-hiPSC. FIG. 11A shows the fold levels of human APOE mRNA assayed by qRT-PCR using TaqMan assay. FIG. 11B (APOE 3/3) and FIG. 11C (APOE 4/4) show hiPSC shows cells stained with pluripotency markers OCT 4 and NANOG. (FROM GRANT)

FIG. 12A-FIG. 12M show the nuclear envelope markers in isogenic APOE 3/3 and 4/4 hiPSC-derived neurons. FIG. 12A shows the immunocytochemistry for lamin B1 in APOE 3/3 hiPSC-derived neurons while FIG. 12B shows lamin B1 staining in APOE 4/4 hiPSC-derived neurons. As demonstrated in FIG. 12C, the quantification of the nuclear envelope circularity showed loss circularity in the APOE 4/4 hiPSC-derived neurons vs. the APOE 3/3 hiPSC-derived neurons before heat treatment while FIG. 12D shows the same comparison after heat treatment. FIG. 12E shows the immunocytochemistry for lamin AC in APOE 3/3 hiPSC-derived neurons while FIG. 12F shows lamin B1 staining in APOE 4/4 hiPSC-derived neurons. FIG. 12G shows the proportion of cells with abnormal nuclear morphology in the APOE 4/4 hiPSC-derived neurons vs. the APOE 3/3 hiPSC-derived neurons before heat treatment while FIG. 12H shows the same comparison after heat treatment. As shown in FIG. 12I, osmotic stress showed an increased sensitivity of the nuclear envelope in the APOE 4/4 neurons compared to the APOE 3/3. FIG. 13J shows the decrease in global 5-mC % in APOE 4/4 hiPSC-derived neurons as compared to APOE 3/3 hiPSC-derived neurons. FIG. 12K and FIG. 12L shows the nuclear leakage as assessed by a dextran assay using 155 kDa fluorescently-label molecule APOE 3/3 hiPSC-derived neurons and 4/4 hiPSC-derived neurons, respectively. FIG. 12M shows the percentage of leaky nuclei for both APOE 3/3 and APO 4/4 hiPSC-derived neurons.

FIG. 13A-FIG. 13E shows the methylation profile of the APOE LD region in isogenic APOE hiPSC-derived neurons. FIG. 13A shows a map of MethylEPIC array probes in chromosome 19 from 45,393,000-45,424,000 (hg19). Those probes with >0.5 methylation levels are highlighted in red. Those probes with <0.5 methylation levels are highlighted in blue. Significant differences in methylation between the APOE neuronal lines are shown using asterisks as follows: black asterisk (>0.1) and red asterisk (>0.2). Because the APOE promoter region was hypomethylated, it became an excellent target region for enhancement of DNA-methylation. FIG. 13B shows a schematic representation of the 27 CpG islands for pyrosequencing in the APOE region, i.e., chromosome 19 from 45,411,858-45,412,079 (hg19). FIG. 13C shows those probes that had significant differences in DNA-methylation levels between isogenic APOE hiPSC-derived neurons. FIG. 13D shows the methylation level (%) of the CpG 11-38 that was quantitatively determined in the isogenic hiPSC-derived neurons using pyrosequencing. FIG. 13E shows a comparison of the methylation level (%) of CpG 11-38 between hiPSC-derived neurons and NeuN⁺ FANS-sorted nuclei using pyrosequencing. Here, the DNA-methylation profiles of the hiPSC-derived neurons were comparable to those observed for the human brain sorted neuronal nuclei (indicating that the hiPSC-derived neuronal system was suitable for drug discovery studies aiming at DNA-methylation editing).

FIG. 14A-FIG. 14D show the AD-related phenotypes in isogenic APOE 3/3 and 4/4 hiPSC-derived neurons. FIG. 14A shows the ratio of extracellular A042:AD40 secreted from APO 3/3 and APOE 4/4 neurons measured by ELISA. FIG. 14B shows the total tau levels measured by ELISA. FIG. 14C shows the neurite outgrowth evaluated using TUBB3 immunostaining in APOE 3/3 hiPSC-derived neurons. and FIG. 14D shows the neurite outgrowth evaluated using TUBB3 immunostaining in APOE 4/4 hiPSC-derived neurons.

FIG. 15A-15B shows methylation in the target promoter region of APOE and the design of gRNA for targeting. FIG. 15A shows the genome browser view of a map of the targeted region using UCSC genome browser viewer. The black bars in the upper portion of the panel shows the positions of (i) the target region, (ii) the designed gRNAs, and (iii) MethylEpic probes. The lower panel of FIG. 15A shows the APOE gene structure including the promoter, exon 1, intron 1, and the TSS. FIG. 15B shows the analysis of DNA-methylation within the APOE-promoter target region. Relevant probes were those that overlapped the target region and showed differences in DNA-methylation levels between the isogenic APOE hiPSC-derived neurons.

FIG. 16 shows the targeting of the promoter region of APOE with gRNA-dCas9-DNMT3A lentiviral vector system. hiPSC-derived cholinergic neurons homozygote to the APOE e4 allele (APOE 4/4) were stably transduced with lentiviral vector carrying gRNA3 paired with either a dCas9-DNMT3A vector or a dCAS9-DNMT3A null vector. To assess the level of APOE mRNA knockdown following the transduction, qRT-PCR was used.

FIG. 17 shows the targeting of the promoter region of APOE with gRNA-dCas9-DNMT3A lentiviral vector system. hiPSC-derived cholinergic neurons homozygote to the APOE e4 allele (APOE 4/4) were stably transduced with lentiviral vector carrying gRNAs 1-4 paired with dCas9-DNMT3A or a dCas9-DNMT3A vector with no-gRNA. To assess the level of APOE mRNA knockdown following the transduction, qRT-PCR was used.

FIG. 18 shows the targeting of the promoter region of APOE with gRNA-dCas9-DNMT3A lentiviral vector system. hiPSC-derived cholinergic neurons homozygote to the APOE e3 allele (APOE 3/3) were stably transduced with lentiviral vector carrying gRNAs 1-4 paired with dCas9-DNMT3A compared to dCAS9-DNMT3A vector with no-gRNA. To assess the level of APOE mRNA knockdown following the transduction, qRT-PCR was used.

FIG. 19 shows the targeting exon 4 region of APOE with a gRNA-dVRER-DNMT3A lentiviral vector system. hiPSC-derived cholinergic neurons homozygote to the APOE e4 allele (APOE 4/4) were stably transduced with lentiviral vector carrying a gRNA 2′-paired with dVRER-DNMT3A compared to a dVRER-DNMT3A vector with no-gRNA. Real-time PCR assessed the level of mRNA knockdown following the transduction. A 15% reduction in the level of APOE-mRNA was observed following transduction with the lentiviral vector carrying the gRNA.

FIG. 20 shows the targeting exon 4 region of APOE with a gRNA-dVRER-DNMT3A lentiviral vector system. hiPSC-derived cholinergic neurons homozygote to the APOE e3 allele (APOE 3/3) were stably transduced with lentiviral vector carrying a gRNA 2′-paired with dVRER-DNMT3A compared to a dVRER-DNMT3A vector with no-gRNA. Real-time PCR assessed the level of mRNA knockdown following the transduction. No changes in APOE-mRNA were observed.

FIG. 21A-FIG. 21B show the schematic strategy to silence APOEe4 allele using DNMT3A-DNMT3L enzymes and KRAB repressor as the effector molecules. FIG. 21 shows a schematic representation of the APOE gene including promoter region and exons 1-4. As shown on the right, two lentiviral vector systems were established. The first system carried dCAS9-gRNA-to-promoter. This vector also contained a SunTag epitope that was recognized by single-chain scFv protein. The second system carried dVRER and a gRNA for specific targeting of SNP rs429358 in the exon 4 (on the e4) and DNMT3A-DNMT3L effectors. gRNA introduced with MS2 binding sites allowed the recruitment of KRAB repressor via the MS2-protein (fusion). FIG. 21B shows that following lentiviral vector delivery of dCAS9-gRNA-SunTag binds to the promoter region on both alleles. However, it was inactive on the e3-allele as it lacked the effector molecules. The recruitment of dVRER via specific binding mediated throughout the recognition of the PAM (NGCG) brings the effector molecules in the action. Following interaction between SunTag-scFv DNA on the e4 will be looped out and two the effector molecules, KRAB and DNMT3A-L repress and methylate the promoter of the e4. This SunTag-MS2-bridging system allows specific repression of the e4 allele.

FIG. 22 shows the schematic of a lentiviral vector carrying gRNA-dCas9/dVRER-repressor transgene. the targeting exon 4 region of APOE with a gRNA-dVRER-DNMT3A lentiviral vector system. The vector backbone was optimized by inclosing Sp1 binding sites. dCas9-KRAB/MeCP2/KRAB-MeCP2 fusion was expressed from EFS-NC promoter. Human U6 promoter drove the gRNA expression. The vector carried gRNA to target the regulatory element within exon 4 overlapping the e4-SNP (i.e., specifically target the ApoE4 allele).

FIG. 23A-FIG. 23B show the targeting exon 4 region of APOE with a gRNA-dVRER-DNMT3A lentiviral vector system. FIG. 23A shows that the construct was identical to that of FIG. 5 but for the addition of the repressor to the fused domains of KRAB-MeCP2. FIG. 23B shows the mRNA level in hiPSC-derived cholinergic neurons homozygote to the APOE e4 allele following stable transduction with lentiviral vector carrying a gRNA 2′-paired with dVRER-CRAB MeCp2 or a lentiviral vector carrying a dVRER-KRAB MeCp2 vector with no gRNA. Real-time PCR assessed the levels of mRNA knockdown following the transduction. The vector harboring gRNA2 caused a >50% reduction in the level of APOE mRNA.

### V. BRIEF SUMMARY

Disclosed herein is an isolated nucleic acid molecule comprising a nucleic acid sequence encoding (i) a Cas endonuclease, (ii) at least one polypeptide having an enzymatic activity, and (iii) at least one guide RNA. Disclosed herein is an isolated nucleic acid molecule comprising a nucleic acid sequence encoding (i) a fusion protein and (ii) at least one guide RNA.

Disclosed herein is a viral vector comprising a disclosed isolated nucleic acid molecule. Disclosed herein is a viral vector comprising a disclosed isolated nucleic acid molecule comprising a nucleic acid sequence encoding (i) a Cas endonuclease, (ii) at least one polypeptide having an enzymatic activity, and (iii) at least one guide RNA. Disclosed herein is a viral vector comprising a disclosed isolated nucleic acid molecule comprising a nucleic acid sequence encoding (i) a fusion protein and (ii) at least one guide RNA.

Disclosed herein is a lentiviral vector comprising an isolated nucleic acid molecule comprising a nucleic acid sequence encoding (i) a Cas endonuclease, (ii) at least one polypeptide having an enzymatic activity, and (iii) at least one guide RNA. Disclosed herein is a lentiviral vector comprising an isolated nucleic acid molecule comprising a nucleic acid sequence encoding (i) a fusion protein and (ii) at least one guide RNA.

Disclosed herein is pharmaceutical formulation comprising a disclosed isolated nucleic acid molecule and a pharmaceutically acceptable carrier. Disclosed herein is pharmaceutical formulation comprising an isolated nucleic acid molecule comprising a nucleic acid sequence encoding (i) a Cas endonuclease, (ii) at least one polypeptide having an enzymatic activity, and (iii) at least one guide RNA, and a pharmaceutically acceptable carrier. Disclosed herein is pharmaceutical formulation comprising an isolated nucleic acid molecule comprising a nucleic acid sequence encoding (i) a fusion protein and (ii) at least one guide RNA, and a pharmaceutically acceptable carrier. Disclosed herein is pharmaceutical formulation comprising a disclosed vector and a pharmaceutically acceptable carrier. Disclosed herein is pharmaceutical formulation comprising a disclosed lentiviral vector and a pharmaceutically acceptable carrier.

Disclosed herein is a host cell comprising an isolated nucleic acid molecule comprising a nucleic acid sequence encoding (i) a Cas endonuclease, (ii) at least one polypeptide having an enzymatic activity, and (iii) at least one guide RNA.

Disclosed herein is a host cell comprising an isolated nucleic acid molecule comprising a nucleic acid sequence encoding (i) a fusion protein and (ii) at least one guide RNA.

Disclosed herein is a host cell comprising a lentiviral vector comprising an isolated nucleic acid molecule comprising a nucleic acid sequence encoding (i) a Cas endonuclease, (ii) at least one polypeptide having an enzymatic activity, and (iii) at least one guide RNA. Disclosed herein is a host cell comprising a lentiviral vector comprising an isolated nucleic acid molecule comprising a nucleic acid sequence encoding (i) a fusion protein and (ii) at least one guide RNA.

Disclosed herein is a host cell comprising plasmid comprising the sequence set forth in any one of SEQ ID NO:21-24, SEQ ID NO:29-36, SEQ ID NO:43-50, SEQ ID NO:53-56, SEQ ID NO:59-61. Disclosed herein is a guide RNA comprising the sequence set forth in any one of SEQ ID NO:05-SEQ ID NO:14. Disclosed herein is a guide RNA comprising the sequence set forth in any one of SEQ ID NO:25-SEQ ID NO:28. Disclosed herein is a guide RNA comprising the sequence set forth in any one of SEQ ID NO:39-SEQ ID NO:42. Disclosed herein is a guide RNA comprising the sequence set forth in any one of SEQ ID NO:51-SEQ ID NO:52. Disclosed herein is a plasmid comprising the sequence set forth in any of SEQ ID NO:21-SEQ ID NO:24.

Disclosed herein is a plasmid comprising the sequence set forth in any of SEQ ID NO:29-SEQ ID NO:36. Disclosed herein is a plasmid comprising the sequence set forth in any of SEQ ID NO:43-SEQ ID NO:50. Disclosed herein is a plasmid comprising the sequence set forth in any of SEQ ID NO:53-SEQ ID NO:56. Disclosed herein is a plasmid comprising the sequence set forth in any of SEQ ID NO:59-SEQ ID NO:61.

Disclosed herein is a method of administering precision gene therapy, the method comprising contacting one or more cells with a therapeutically effective amount of a viral vector, wherein the viral vector comprises an isolated nucleic acid molecule comprising a nucleic acid sequence encoding (i) a fusion protein and (ii) at least one guide RNA, wherein the fusion protein comprises a Cas endonuclease and a polypeptide having an enzymatic activity, and reducing expression of APOE in one or more cells.

Disclosed herein is a method of administering precision gene therapy, the method comprising contacting one or more cells with a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, and reducing expression of APOE in one or more cells.

Disclosed herein is a method of administering precision gene therapy, the method comprising contacting one or more cells with a therapeutically effective amount of a viral vector, wherein the viral vector comprises an isolated nucleic acid molecule comprising a nucleic acid sequence encoding (i) a fusion protein and (ii) at least one guide RNA, wherein the fusion protein comprises a Cas endonuclease and a polypeptide having an enzymatic activity, and reducing expression of the APOE e4 allele in one or more cells.

Disclosed herein is a method of administering precision gene therapy, the method comprising contacting one or more cells with a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, and reducing expression of the APOE e4 allele in one or more cells.

Disclosed herein is a method of administering precision gene therapy, the method comprising administering to a subject in need thereof a therapeutically effective amount of a viral vector, wherein the viral vector comprises an isolated nucleic acid molecule comprising a nucleic acid sequence encoding (i) a fusion protein and (ii) at least one guide RNA, wherein the fusion protein comprises a Cas endonuclease and a polypeptide having an enzymatic activity, and reducing expression of the APOE e4 allele.

Disclosed herein is a method of administering precision gene therapy, the method comprising administering to a subject in need thereof a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, and reducing expression of the APOE e4 allele.

Disclosed herein is a method of treating and/or preventing Alzheimer's disease progression in a subject, the method comprising administering to a subject in need thereof a therapeutically effective amount of a viral vector, wherein the viral vector comprises an isolated nucleic acid molecule comprising a nucleic acid sequence encoding (i) a fusion protein comprising a Cas endonuclease and a polypeptide having an enzymatic activity and (ii) at least one guide RNA, and reducing expression of APOE, thereby reducing the pathological phenotype associated with Alzheimer's disease.

Disclosed herein is a method of treating and/or preventing Alzheimer's disease progression in a subject, the method comprising reducing the pathological phenotype associated with Alzheimer's disease by administering to a subject in need thereof a therapeutically effective amount of a viral vector, wherein the viral vector comprises an isolated nucleic acid molecule comprising a nucleic acid sequence encoding (i) a fusion protein comprising a Cas endonuclease and a polypeptide having an enzymatic activity and (ii) at least one guide RNA, and reducing expression of APOE.

Disclosed herein is a method of treating and/or preventing Alzheimer's disease progression in a subject, the method comprising administering to a subject in need thereof a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, and reducing expression of APOE, thereby reducing the pathological phenotype associated with Alzheimer's disease.

Disclosed herein is a method of treating and/or preventing Alzheimer's disease progression in a subject, the method comprising reducing the pathological phenotype associated with Alzheimer's disease by administering to a subject in need thereof a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, and reducing expression of APOE.

Disclosed herein is a method of treating and/or preventing Alzheimer's disease progression in a subject, the method comprising administering to a subject in need thereof a therapeutically effective amount of a viral vector, wherein the viral vector comprises an isolated nucleic acid molecule comprising a nucleic acid sequence encoding (i) a fusion protein comprising a Cas endonuclease and a polypeptide having an enzymatic activity and (ii) at least one guide RNA, and reducing expression of the APOE e4 allele, thereby reducing the pathological phenotype associated with Alzheimer's disease.

Disclosed herein is a method of treating and/or preventing Alzheimer's disease progression in a subject, the method comprising reducing the pathological phenotype associated with Alzheimer's disease by administering to a subject in need thereof a therapeutically effective amount of a viral vector, wherein the viral vector comprises an isolated nucleic acid molecule comprising a nucleic acid sequence encoding (i) a fusion protein comprising a Cas endonuclease and a polypeptide having an enzymatic activity and (ii) at least one guide RNA, and reducing expression of the APOE e4 allele.

Disclosed herein is a method of treating and/or preventing Alzheimer's disease progression in a subject, the method comprising administering to a subject in need thereof a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, and reducing expression of the APOE e4 allele, thereby reducing the pathological phenotype associated with Alzheimer's disease.

Disclosed herein is a method of treating and/or preventing Alzheimer's disease progression in a subject, the method comprising reducing the pathological phenotype associated with Alzheimer's disease by administering to a subject in need thereof a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, and reducing expression of the APOE e4 allele.

Disclosed herein is a method of reducing expression of APOE, the method comprising administering to a subject in need thereof a therapeutically effective amount of a viral vector, wherein the viral vector comprises an isolated nucleic acid molecule comprising a nucleic acid sequence encoding (i) a fusion protein comprising a Cas endonuclease and a polypeptide having an enzymatic activity and (ii) at least one guide RNA, thereby reducing expression of APOE.

Disclosed herein is a method of reducing expression of APOE, the method comprising administering to a subject in need thereof a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, thereby reducing expression of APOE.

Disclosed herein is a method of reducing expression of APOE e4, the method comprising administering to a subject in need thereof a therapeutically effective amount of a viral vector, wherein the viral vector comprises an isolated nucleic acid molecule comprising a nucleic acid sequence encoding (i) a fusion protein comprising a Cas endonuclease and a polypeptide having an enzymatic activity and (ii) at least one guide RNA, thereby reducing expression of the APOE e4 allele.

Disclosed herein is a method of reducing expression of APOE e4, the method comprising administering to a subject in need thereof a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, thereby reducing expression of the APOE e4 allele.

Disclosed herein is a kit comprising one or more disclosed isolated nucleic acid molecules, disclosed vectors, disclosed lentiviral vectors, disclosed pharmaceutical formulations, disclosed host cells, disclosed guide RNAs, disclosed plasmids, or any combination thereof with or without additional therapeutic agents to treat, prevent, inhibit, and/or ameliorate one or more symptoms or complications associated AD or LOAD.

### VI. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Relevant Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is +10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have Alzheimer's disease (e.g., LOAD), be suspected of having Alzheimer's disease, or be at risk of developing and/or acquiring Alzheimer's disease.

As used herein, the term “diagnosed” means having been subjected to an examination by a person of skill, for example, a physician, and found to have a condition that can be diagnosed or treated by one or more of the disclosed agents, disclosed therapeutic agents, disclosed pharmaceutical formulations, or a combination thereof, or by one or more of the disclosed methods. For example, “diagnosed with Alzheimer's disease or LOAD” means having been subjected to an examination by a person of skill, for example, a physician, and found to have a condition that can be treated by one or more of the disclosed isolated nucleic acid molecules, disclosed viral vectors, disclosed lentiviral vectors, disclosed pharmaceutical formulations, disclosed host cells, disclosed gRNAs, disclosed plasmids, or any combination thereof, or by one or more of the disclosed methods. For example, “suspected of having Alzheimer's disease” can mean having been subjected to an examination by a person of skill, for example, a physician, and found to have a condition that can likely be treated by one or more of the disclosed isolated nucleic acid molecules, disclosed viral vectors, disclosed lentiviral vectors, disclosed pharmaceutical formulations, disclosed host cells, disclosed gRNAs, disclosed plasmids, or any combination thereof, or by one or more of the disclosed methods. In an aspect, an examination can be physical, can involve various tests (e.g., blood tests, genotyping, biopsies, etc.) and assays (e.g., enzymatic assay), or a combination thereof.

A “patient” can refer to a subject that has been diagnosed with or is suspected of having Alzheimer's disease (AD) or late-onset Alzheimer's disease (LOAD). In an aspect, a patient can refer to a subject that has been diagnosed with or is suspected of having AD such as for example, LOAD, and is seeking treatment or receiving treatment for AD or LOAD.

As used herein, the phrase “identified to be in need of treatment for a disorder,” or the like, refers to selection of a subject based upon need for treatment of the disorder. For example, a subject can be identified as having a need for treatment of a disorder (e.g., such as Alzheimer's disease) based upon an earlier diagnosis by a person of skill and thereafter subjected to treatment for the disorder (e.g., AD or LOAD). In an aspect, the identification can be performed by a person different from the person making the diagnosis. In an aspect, the administration can be performed by one who performed the diagnosis.

As used herein, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having Alzheimer's disease). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels. In an aspect, a native or control level can be a pre-disease or pre-disorder level.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder (such as Alzheimer's disease). In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating Alzheimer's disease or LOAD can reduce the severity of an established disease in a subject by 1%-100% as compared to a control (such as, for example, an individual not having AD or LOAD). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of AD or LOAD. For example, treating Alzheimer's disease can reduce one or more symptoms of AD or LOAD in a subject by 1%-100% as compared to a control (such as, for example, an individual not having AD or LOAD). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established AD (such as LOAD). It is understood that treatment does not necessarily refer to a cure or complete ablation or eradication of AD. However, in an aspect, treatment can refer to a cure or complete ablation or eradication of AD or LOAD.

As used herein, “SunTag” refers to a tag that allows numerous copies of GFP to be recruited to a protein of interest for bright signals. The SunTag can be used for amplification of a fluorescence signal (Tanenbaum M E, et al. (2014) Cell. 159(3):635-646).

As used herein, a “biomarker” refers to a defined characteristic that is measured as an indicator of normal biological processes, pathogenic processes, or response to an exposure of intervention. In an aspect, a biomarker can be diagnostic (i.e., detects or classifies a pathological condition), prognostic (i.e., predicts the probability of disease occurrence or progression), pharmacodynamic/responsive (i.e., identifies a change in response to a therapeutic intervention), predictive (i.e., predicts how an individual or subject might respond to a particular intervention or event). In an aspect, a biomarker can be diagnostic, prognostic, pharmacodynamic/responsive, and/or predictive at the same time. In an aspect, a biomarker can be diagnostic, prognostic, pharmacodynamic/responsive, and/or predictive at different times (e.g., first a biomarker can be diagnostic and then later, the same biomarker can be prognostic, pharmacodynamic/responsive, and/or predictive). A biomarker can be an objective measure that can be linked to a clinical outcome assessment. A biomarker can be used by the skilled person to make a clinical decision based on its context of use.

As used herein, “operably linked” means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing Alzheimer's disease (AD) or LOAD and/or AD or LOAD progression is intended. The words “prevent” and “preventing” and “prevention” also refer to prophylactic or preventative measures for protecting or precluding a subject (e.g., an individual) not having AD or LOAD or an AD or LOAD complication from progressing to that complication. In an aspect, preventing or reducing APOE expression and/or activity is intended.

As used herein, the terms “administering” and “administration” refer to any method of providing one or more of the disclosed isolated nucleic acid molecules, disclosed pharmaceutical formulations, disclosed vectors, or any combination thereof to a subject. Such methods are well known to those skilled in the art and include, but are not limited to, the following routes: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed therapeutic agent, a disclosed pharmaceutical composition, or a combination thereof can comprise administration directly into the CNS (e.g., intraparenchymal, intracerebroventriular, inthrathecal cisternal, intrathecal (lumbar), deep gray matter delivery, convection-enhanced delivery to deep gray matter) or the PNS. Administration can be continuous or intermittent.

In an aspect, a “therapeutic agent” can be a “biologically active agent” or “biologic active agent” or “bioactive agent”, which refers to an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, vaccines, hormones, antibodies (including active antibody fragments sFv, Fv, and Fab fragments), aptamers, peptide mimetics, functional nucleic acids, therapeutic proteins, peptides, or nucleic acids. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents. It is understood that a biologically active agent can be used in connection with administration to various subjects, for example, to humans (i.e., medical administration) or to animals (i.e., veterinary administration). As used herein, the recitation of a biologically active agent inherently encompasses the pharmaceutically acceptable salts thereof.

In an aspect, a “therapeutic agent” can be any agent that effects a desired clinical outcome in a subject having AD or LOAD, suspected of having AD or LOAD, and/or likely to develop or acquire AD or LOAD. In an aspect, a disclosed therapeutic agent can be an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable.

In an aspect, a therapeutic agent can be a “drug” or a “vaccine” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. Examples include but are not limited to a radiosensitizer, the combination of a radiosensitizer and a chemotherapeutic, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, carbonic anhydrase inhibitors, prostaglandin analogs, a combination of an alpha agonist and a beta blocker, a combination of a carbonic anhydrase inhibitor and a beta blocker, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, or a vaccine. In a further aspect, the pharmaceutically active agent can be coumarin, albumin, bromolidine, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, timol hemihydrate, levobunolol hydrochloride, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists (i.e., alpha adrenergic receptor agonist) such as clonidine, brimonidine tartrate, and apraclonidine hydrochloride; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; prostaglandin analogs such as latanoprost, travoprost, and bimatoprost; cholinergics (i.e., acetylcholine receptor agonists) such as pilocarpine hydrochloride and carbachol; glutamate receptor agonists such as the N-methyl D-aspartate receptor agonist memantine; anti-Vascular endothelial growth factor (VEGF) aptamers such as pegaptanib; anti-VEGF antibodies (including but not limited to anti-VEGF-A antibodies) such as ranibizumab and bevacizumab; carbonic anhydrase inhibitors such as methazolamide, brinzolamide, dorzolamide hydrochloride, and acetazolamide; antiarrhythmic agents such as quinidine, lidocaine, tocainide hydrochloride, mexiletine hydrochloride, digoxin, verapamil hydrochloride, propafenone hydrochloride, flecaimide acetate, procainamide hydrochloride, moricizine hydrochloride, and diisopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides. It is understood that a pharmaceutically active agent can be used in connection with administration to various subjects, for example, to humans (i.e., medical administration) or to animals (i.e., veterinary administration). As used herein, the recitation of a pharmaceutically active agent inherently encompasses the pharmaceutically acceptable salts thereof.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of glycogen branching enzymes and amylases. It should be understood that sequence with substantial sequence identity do not necessarily have the same length and may differ in length. For example, sequences that have the same nucleotide sequence but of which one has additional nucleotides on the 3′- and/or 5′-side are 100% identical.

In an aspect, the skilled person can determine an efficacious dose, an efficacious schedule, and an efficacious route of administration for one or more of the disclosed isolated nucleic acid molecules, disclosed pharmaceutical formulations, disclosed vectors, or any combination thereof so as to treat or prevent AD or LOAD. In an aspect, the skilled person can also alter, change, or modify an aspect of an administering step to improve efficacy of one or more of the disclosed isolated nucleic acid molecules, disclosed pharmaceutical formulations, disclosed vectors, or any combination thereof. In an aspect, the skilled person can determine an efficacious dose, an efficacious schedule, and an efficacious route of administration for any disclosed isolated nucleic acid molecule, disclosed pharmaceutical formulation, disclosed vector, disclosed therapeutic agent, or any combination thereof.

As used herein, “modifying the method” can comprise modifying or changing one or more features or aspects of one or more steps of a disclosed method. For example, in an aspect, a method can be altered by changing the amount of one or more of the disclosed isolated nucleic acid molecules, disclosed viral vectors, disclosed lentiviral vectors, disclosed pharmaceutical formulations, disclosed host cells, disclosed gRNAs, disclosed plasmids, or any combination thereof, or administered to a subject, or by changing the frequency of administration of one or more of the disclosed isolated nucleic acid molecules, disclosed viral vectors, disclosed lentiviral vectors, disclosed pharmaceutical formulations, disclosed host cells, disclosed gRNAs, disclosed plasmids, or any combination thereof, or by changing the duration of time that the one or more of the disclosed isolated nucleic acid molecules, disclosed viral vectors, disclosed lentiviral vectors, disclosed pharmaceutical formulations, disclosed host cells, disclosed gRNAs, disclosed plasmids, or any combination thereof, or are administered to a subject.

As used herein, “concurrently” means (1) simultaneously in time, or (2) at different times during the course of a common treatment schedule.

The term “contacting” as used herein refers to bringing one or more of disclosed isolated nucleic acid molecules, disclosed pharmaceutical formulations, disclosed vectors, or any combination thereof together with a target area or intended target area in such a manner that the one or more of the disclosed isolated nucleic acid molecules, disclosed pharmaceutical formulations, disclosed vectors, or any combination thereof exert an effect on the intended target or targeted area either directly or indirectly. A target area or intended target area can be one or more of a subject's organs (e.g., lungs, heart, liver, kidney, brain, etc.). In an aspect, a target area or intended target area can be any cell or any organ infected by AD or LOAD (such as cholinergic neurons). In an aspect, a target area or intended target area can be the brain or various neuronal populations.

As used herein, “determining” can refer to measuring or ascertaining the presence and severity of AD such as, for example, LOAD. Methods and techniques used to determine the presence and/or severity of AD are typically known to the medical arts. For example, the art is familiar with the ways to identify and/or diagnose the presence, severity, or both of AD (such as, for example, a LOAD. In an aspect, “determining” can also refer to measuring or ascertaining the level of one or more proteins or peptides in a biosample, or measuring or ascertaining the level or one or more RNAs or miRNAs in a biosample. Methods and techniques for determining the level of proteins/peptides and RNAs/miRNAs are known to the art and are disclosed herein.

As used herein, “effective amount” and “amount effective” can refer to an amount that is sufficient to achieve the desired result such as, for example, the treatment and/or prevention of AD or LOAD. As used herein, the terms “effective amount” and “amount effective” can refer to an amount that is sufficient to achieve the desired an effect on an undesired condition (e.g., a AD or LOAD). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed isolated nucleic acid molecule, a disclosed pharmaceutical formulation, a disclosed vector, or any combination thereof that (i) treats the particular disease, condition, or disorder (e.g., AD or LOAD), (ii) attenuates, ameliorates, or eliminates one or more symptoms of the particular disease, condition, or disorder (e.g., AD or LOAD), or (iii) delays the onset of one or more symptoms of the particular disease, condition, or disorder described herein (e.g., AD or LOAD). The specific therapeutically effective dose level for any particular patient will depend upon a variety of factors including the disorder being treated and the severity of the disorder; the isolated nucleic acid molecules, disclosed pharmaceutical formulations, disclosed vectors, or any combination thereof employed; the disclosed methods employed; the age, body weight, general health, sex and diet of the patient; the time of administration; the route of administration; the rate of excretion of the disclosed isolated nucleic acid molecules, disclosed pharmaceutical formulations, disclosed vectors, or any combination thereof employed; the duration of the treatment; drugs used in combination or coincidental with the disclosed isolated nucleic acid molecules, disclosed pharmaceutical formulations, disclosed vectors, or any combination thereof employed, and other like factors well known in the medical arts. For example, it is well within the skill of the art to start doses of the disclosed isolated nucleic acid molecules, disclosed pharmaceutical formulations, disclosed vectors, or any combination thereof at levels lower than those required to achieve the desired therapeutic effect and to gradually increase the dosage until the desired effect is achieved. If desired, then the effective daily dose can be divided into multiple doses for purposes of administration. Consequently, a single dose of the disclosed isolated nucleic acid molecules, disclosed pharmaceutical formulations, disclosed vectors, or any combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, AD or LOAD

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of one or more of the disclosed agents, disclosed therapeutic agents, disclosed pharmaceutical formulations or a combination thereof includes the use of more than one therapy (e.g., additional therapeutic agents). Administration “in combination with” one or more additional therapeutic agents includes simultaneous (e.g., concurrent) and consecutive administration in any order. The use of the term “in combination” does not restrict the order in which therapies are administered to a subject. By way of non-limiting example, a first therapy (e.g., one or more of the disclosed isolated nucleic acid molecules, disclosed pharmaceutical formulations, disclosed vectors, or any combination thereof) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., one or more of the disclosed isolated nucleic acid molecules, disclosed pharmaceutical formulations, disclosed vectors, or any combination thereof or one or more additional therapeutic agents) to a subject having or diagnosed with AD or LOAD.

As used herein, “CRISPR or clustered regularly interspaced short palindromic repeat” is an ideal tool for correction of genetic abnormalities associated with diseases such as Alzheimer's disease or LOAD. The system can be designed to target genomic DNA directly. A CRISPR system involves two main components: a Cas9 enzyme and a guide (gRNA). The gRNA contains a targeting sequence for DNA binding (at, for example, the APOE promoter region) and a scaffold sequence for Cas9 binding. Cas9 nuclease is often used to “knockout” target genes such as for example, the APOE e4 allele. Also, multiple gRNAs can be employed to suppress or activate multiple genes simultaneously, hence increasing the treatment efficacy and reducing resistance potentially caused by new mutations in the target genes.

As used herein, “CRISPR-based endonucleases” include RNA-guided endonucleases that comprise at least one nuclease domain and at least one domain that interacts with a guide RNA. As known to the art, a guide RNA directs the CRISPR-based endonucleases to a targeted site in a nucleic acid at which site the CRISPR-based endonucleases cleaves at least one strand of the targeted nucleic acid sequence. As the guide RNA provides the specificity for the targeted cleavage, the CRISPR-based endonuclease is universal and can be used with different guide RNAs to cleave different target nucleic acid sequences. CRISPR-based endonucleases are RNA-guided endonucleases derived from CRISPR/Cas systems.

In an aspect, a disclosed CRISPR-based endonuclease can be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In an aspect, a disclosed CRISPR-based endonuclease can be derived from a type II CRISPR/Cas system. For example, in an aspect, a CRISPR-based endonuclease can be derived from a Cas9 protein. The Cas9 protein can be from Streptococcus pyogenes, Streptococcus thermophilus, Streptococcus sp, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. In an aspect, the CRISPR-based nuclease can be derived from a Cas9 protein from Streptococcus pyogenes. In an aspect, the CRISPR-based nuclease can comprise the sequence set forth in SEQ ID NO:64 or SEQ ID NO:65.

As used herein, “CRISPRa” refers to CRISPR Activation, which is using a dCas9 or dCas9-activator with a gRNA to increase transcription of a target gene.

As used herein, “CRISPRi” refers to CRISPR Interference, which is using a dCas9 or dCas9-repressor with a gRNA to repress/decrease transcription of a target gene.

As used herein, “dCas9” refers to enzymatically inactive form of Cas9, which can bind, but cannot cleave, DNA.

As used herein, “Protospacer Adjacent Motif” or “PAM” refers to a sequence adjacent to the target sequence that is necessary for Cas enzymes to bind target DNA.

Disclosed are the components to be used to prepare the disclosed isolated nucleic acid molecules, disclosed pharmaceutical formulations, disclosed vectors, or any combination thereof as well the disclosed isolated nucleic acid molecules, disclosed pharmaceutical formulations, disclosed vectors, or any combination thereof used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific aspects or combination of aspects of the disclosed methods.

### B. Compositions

### 1. Isolated Nucleic Acid Molecules

Disclosed herein is an isolated nucleic acid molecule comprising a nucleic acid sequence encoding (i) a Cas endonuclease, (ii) at least one polypeptide having an enzymatic activity, and (iii) at least one guide RNA.

In an aspect, a disclosed Cas endonuclease can comprise Cas9, SpCas9, SaCas9, a variant Cas9, a dCas, or a dCas9. In an aspect, a disclosed Cas9 can comprise the sequence set forth in SEQ ID NO:64 or SEQ ID NO:65. In an aspect, a disclosed Cas9 can have a sequence having at least 75%, at least 80%, at least 85%, at least 90%, or at least 95% identity to the sequence set forth in SEQ ID NO:64 or SEQ ID NO:65 or a fragment thereof.

In an aspect, a disclosed variant Cas9 can comprise VQR, EQR, or VRER. In an aspect a disclosed VRER can comprise the sequence set forth in SEQ ID NO:15. In an aspect, a disclosed VRER can have a sequence having at least 75%, at least 80%, at least 85%, at least 90%, or at least 95% identity to the sequence set forth in SEQ ID NO:15 or a fragment thereof. In an aspect, a disclosed dCas can comprise dVQR, dEQR, or dVRER. In an aspect, a disclosed dCas can comprise the sequence set forth in SEQ ID NO:16. In an aspect, a disclosed dCas9 can have a sequence having at least 75%, at least 80%, at least 85%, at least 90%, or at least 95% identity to the sequence set forth in SEQ ID NO:16 or a fragment thereof. A SpCas9 (3′NGG-PAM sequence) can comprise SpCas9 VQR (3′NGAN or 3′NGNG), SpCas9 EQR (3′NGAG), or SpCas9 VRER (3′NGCG).

In an aspect, a disclosed VRER can have the sequence set forth below or a sequence having 70%, 75%, 80%, 85%, 90%, 95%, or more identity to the sequence set forth below:

(SEQ ID NO: 15)
atggataaaaagtattctattggtttagacatcggcactaattccgttggatgggctgtcataaccgatgaatacaaagtaccttcaaagaaatt
taaggtgttggggaacacagaccgtcattcgattaaaaagaatcttatcggtgccctcctattcgatagtggcgaaacggcagaggcgactc
gcctgaaacgaaccgctcggagaaggtatacacgtcgcaagaaccgaatatgttacttacaagaaatttttagcaatgagatggccaaagtt
gacgattctttctttcaccgtttggaagagtccttccttgtcgaagaggacaagaaacatgaacggcaccccatctttggaaacatagtagatg
aggtggcatatcatgaaaagtacccaacgatttatcacctcagaaaaaagctagttgactcaactgataaagcggacctgaggttaatctact
tggctcttgcccatatgataaagttccgtgggcactttctcattgagggtgatctaaatccggacaactcggatgtcgacaaactgttcatcca
gttagtacaaacctataatcagttgtttgaagagaaccctataaatgcaagtggcgtggatgcgaaggctattcttagcgcccgcctctctaaa
tcccgacggctagaaaacctgatcgcacaattacccggagagaagaaaaatgggttgttcggtaaccttatagcgctctcactaggcctga
caccaaattttaagtcgaacttcgacttagctgaagatgccaaattgcagcttagtaaggacacgtacgatgacgatctcgacaatctactgg
cacaaattggagatcagtatgcggacttatttttggctgccaaaaaccttagcgatgcaatcctcctatctgacatactgagagttaatactgag
attaccaaggcgccgttatccgcttcaatgatcaaaaggtacgatgaacatcaccaagacttgacacttctcaaggccctagtccgtcagca
actgcctgagaaatataaggaaatattctttgatcagtcgaaaaacgggtacgcaggttatattgacggcggagcgagtcaagaggaattct
acaagtttatcaaacccatattagagaagatggatgggacggaagagttgcttgtaaaactcaatcgcgaagatctactgcgaaagcagcg
gactttcgacaacggtagcattccacatcaaatccacttaggcgaattgcatgctatacttagaaggcaggaggatttttatccgttcctcaaa
gacaatcgtgaaaagattgagaaaatcctaacctttcgcataccttactatgtgggacccctggcccgagggaactctcggttcgcatggat
gacaagaaagtccgaagaaacgattactccatggaattttgaggaagttgtcgataaaggtgcgtcagctcaatcgttcatcgagaggatga
ccaactttgacaagaatttaccgaacgaaaaagtattgcctaagcacagtttactttacgagtatttcacagtgtacaatgaactcacgaaagtt
aagtatgtcactgagggcatgcgtaaacccgcctttctaagcggagaacagaagaaagcaatagtagatctgttattcaagaccaaccgca
aagtgacagttaagcaattgaaagaggactactttaagaaaattgaatgcttcgattctgtcgagatctccggggtagaagatcgatttaatgc
gtcacttggtacgtatcatgacctcctaaagataattaaagataaggacttcctggataacgaagagaatgaagatatcttagaagatatagtg
ttgactcttaccctctttgaagatcgggaaatgattgaggaaagactaaaaacatacgctcacctgttcgacgataaggttatgaaacagttaa
agaggcgtcgctatacgggctggggacgattgtcgcggaaacttatcaacgggataagagacaagcaaagtggtaaaactattctcgattt
tctaaagagcgacggcttcgccaataggaactttatgcagctgatccatgatgactctttaaccttcaaagaggatatacaaaaggcacaggt
ttccggacaaggggactcattgcacgaacatattgcgaatcttgctggttcgccagccatcaaaaagggcatactccagacagtcaaagta
gtggatgagctagttaaggtcatgggacgtcacaaaccggaaaacattgtaatcgagatggcacgcgaaaatcaaacgactcagaaggg
gcaaaaaaacagtcgagagcggatgaagagaatagaagagggtattaaagaactgggcagccagatcttaaaggagcatcctgtggaa
aatacccaattgcagaacgagaaactttacctctattacctacaaaatggaagggacatgtatgttgatcaggaactggacataaaccgtttat
ctgattacgacgtcgatcacattgtaccccaatcctttttgaaggacgattcaatcgacaataaagtgcttacacgctcggataagaaccgag
ggaaaagtgacaatgttccaagcgaggaagtcgtaaagaaaatgaagaactattggcggcagctcctaaatgcgaaactgataacgcaaa
gaaagttcgataacttaactaaagctgagaggggggcttgtctgaacttgacaaggccggatttattaaacgtcagctcgtggaaacccgc
caaatcacaaagcatgttgcacagatactagattcccgaatgaatacgaaatacgacgagaacgataagctgattcgggaagtcaaagtaa
tcactttaaagtcaaaattggtgtcggacttcagaaaggattttcaattctataaagttagggagataaataactaccaccatgcgcacgacgc
ttatcttaatgccgtcgtagggaccgcactcattaagaaatacccgaagctagaaagtgagtttgtgtatggtgattacaaagtttatgacgtcc
gtaagatgatcgcgaaaagcgaacaggagataggcaaggctacagccaaatacttcttttattctaacattatgaatttctttaagacggaaat
cactctggcaaacggagagatacgcaaacgacctttaattgaaaccaatggggagacaggtgaaatcgtatgggataagggccgggactt
cgcgacggtgagaaaagttttgtccatgccccaagtcaacatagtaaagaaaactgaggtgcagaccggagggttttcaaaggaatcgatt
cttccaaaaaggaatagtgataagctcatcgctcgtaaaaaggactgggacccgaaaaagtacggtggcttcgtgagccctacagttgcct
attctgtcctagtagtggcaaaagttgagaagggaaaatccaagaaactgaagtcagtcaaagaattattggggataacgattatggagcgc
tcgtcttttgaaaagaaccccatcgacttccttgaggcgaaaggttacaaggaagtaaaaaaggatctcataattaaactaccaaagtatagt
ctgtttgagttagaaaatggccgaaaacggatgttggctagcgccagagagcttcaaaaggggaacgaactcgcactaccgtctaaatacg
tgaatttcctgtatttagcgtcccattacgagaagttgaaaggttcacctgaagataacgaacagaagcaactttttgttgagcagcacaaaca
ttatctcgacgaaatcatagagcaaatttcggaattcagtaagagagtcatcctagctgatgccaatctggacaaagtattaagcgcatacaa
caagcacagggataaacccatacgtgagcaggcggaaaatattatccatttgtttactcttaccaacctcggcgctccagccgcattcaagta
ttttgacacaacgatagatcgcaaagagtacagatctaccaaggaggtgctagacgcgacactgattcaccaatccatcacgggattatatg
aaactcggatagatttgtcacagcttgggggtgactga.

In an aspect, dCas9 can have the following sequence:

(SEQ ID NO: 16)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD.

In an aspect, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof. In an aspect, a disclosed encoded polypeptide can be histone deacetylase or heterochromatin protein 1. In an aspect, a disclosed encoded polypeptide can comprise transcription repression activity. In an aspect, a disclosed DNMT3A can have the amino acid sequence set forth in SEQ ID NO:17 or the nucleotide sequence set forth in SEQ ID NO:18. In an aspect, a disclosed DNMT3A can have a sequence having at least 75%, at least 80%, at least 85%, at least 90%, or at least 95% identity to the sequence set forth in SEQ ID NO:17 or SEQ ID NO:18 or a fragment thereof.

In an aspect, a disclosed DNMT3A can have the sequence set forth below or a sequence having 70%, 75%, 80%, 85%, 90%, 95%, or more identity to the sequence set forth:

(SEQ ID NO: 17)
PSRLQMFFANNHDQEFDPPKVYPPVPAEKRKPIRVLSLFDGIATGLLV
LKDLGIQVDRYIASEVCEDSITVGMVRHQGKIMYVGDVRSVTQKHIQE
WGPFDLVIGGSPCNDLSIVNPARKGLYEGTGRLFFEFYRLLHDARPKE
GDDRPFFWLFENVVAMGVSDKRDISRFLESNPVMIDAKEVSAAHRARY
FWGNLPGMNRPLASTVNDKLELQECLEHGRIAKFSKVRTITTRSNSIK
QGKDQHFPVFMNEKEDILWCTEMERVFGFPVHYTDVSNMSRLARQRLL
GRSWSVPVIRHLFAPLKEYFACV.

In an aspect, a disclosed DNMT3A can have the sequence set forth below or a sequence having 70%, 75%, 80%, 85%, 90%, 95%, or more identity to the sequence set forth:

(SEQ ID NO: 18)
Ccccccggctccagatgttcttcgctaataaccacgaccaggaatttgac
cctccaaaggtttacccacctgtcccagctgagaagaggaagcccatccg
ggtgctgtctctctttgatggaatcgctacagggctcctggtgctgaagg
acttgggcattcaggtggaccgctacattgcctcggaggtgtgtgaggac
tccatcacggtgggcatggtgcggcaccaggggaagatcatgtacgtcgg
ggacgtccgcagcgtcacacagaagcatatccaggagtggggcccattcg
atctggtgattgggggcagtccctgcaatgacctctccatcgtcaaccct
gctcgcaagggcctctacgagggcactggccggctcttctttgagttcta
ccgcctcctgcatgatgcgcggcccaaggagggagatgatcgccccttct
tctggctctttgagaatgtggtggccatgggcgttagtgacaagagggac
atctcgcgatttctcgagtccaaccctgtgatgattgatgccaaagaagt
gtcagctgcacacagggcccgctacttctggggtaaccttcccggtatga
acaggccgttggcatccactgtgaatgataagctggagctgcaggagtgt
ctggagcatggcaggatagccaagttcagcaaagtgaggaccattactac
gaggtcaaactccataaagcagggcaaagaccagcattttcctgtgttca
tgaatgagaaagaggacatcttatggtgcactgaaatggaaagggtattt
ggtttcccagtccactatactgacgtgtccaacatgagccgcttggcgag
gcagagactgctgggccggtcatggagcgtgccagtcatccgccacctct
tcgctccgctgaaggagtattttgcgtgtgtg.

In an aspect, at least one encoded polypeptide can comprise Kroppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed TRD of MeCP2 can comprise the nucleotide sequence set forth in SEQ ID NO:57 or the amino acid sequence set forth in SEQ ID NO:58. In an aspect, a disclosed TRD of MeCP2 can have a sequence having at least 75%, at least 80%, at least 85%, at least 90%, or at least 95% identity to the sequence set forth in SEQ ID NO:57 or SEQ ID NO:58 or a fragment thereof. In an aspect, a disclosed KRAB-MeCP2 can comprise the nucleotide sequence set forth in SEQ ID NO:62 or the amino acid sequence set forth in SEQ ID NO:63. In an aspect, a disclosed KRAB-MeCP2 can have a sequence having at least 75%, at least 80%, at least 85%, at least 90%, or at least 95% identity to the sequence set forth in SEQ ID NO:62 or SEQ ID NO:63 or a fragment thereof.

In an aspect, a disclosed MeCP2 TRD can comprise the sequence set forth below or a sequence having 70%, 75%, 80%, 85%, 90%, 95%, or more identity to the sequence set forth:

(SEQ ID NO: 57)
atgtcggagggtgtgcaggtgaaaagggtcctggagaaaagtcctgggaa
gctccttgtcaagatgccttttcaaacttcgccagggggcaaggctgagg
ggggtggggccaccacatccacccaggtcatggtgatcaaacgccccggc
aggaagcgaaaagctgaggccgaccctcaggccattcccaagaaacgggg
ccgaaagccggggagtgtggtggcagccgctgccgccgaggccaaaaaga
aagccgtgaaggagtcttctatccgatctgtgcaggagacagtactcccc
atcaagaagcgcaagacccgggagtaa.

In an aspect, a disclosed MeCP2 TRD can comprise the sequence set forth below or a sequence having 70%, 75%, 80%, 85%, 90%, 95%, or more identity to the sequence set forth:

(SEQ ID NO: 58)
MSEGVQVKRVLEKSPGKLLVKMPFQTSPGGKAEGGGATTSTQVMVIKRPG
RKRKAEADPQAIPKKRGRKPGSVVAAAAAEAKKKAVKESSIRSVQETVLP
IKKRKTRE*.

In an aspect, a disclosed KRAB-MeCP2 repressor can comprise the sequence set forth below or a sequence having 70%, 75%, 80%, 85%, 90%, 95%, or more identity to the sequence set forth:

(SEQ ID NO: 62)
atgcggacactggtgaccttcaaggatgtatttgtggacttcaccaggga
ggagtggaagctgctggacactgctcagcagatcgtgtacagaaatgtga
tgctggagaactataagaacctggtttccttgggttatcagcttactaag
ccagatgtgatcctccggttggagaagggagaagagccctcgggaggtgg
ttcgggaggtggttcggagggtgtgcaggtgaaaagggtcctggagaaaa
gtcctgggaagctccttgtcaagatgccttttcaaacttcgccagggggc
aaggctgaggggggtggggccaccacatccacccaggtcatggtgatcaa
acgccccggcaggaagcgaaaagctgaggccgaccctcaggccattccca
agaaacggggccgaaagccggggagtgtggtggcagccgctgccgccgag
gccaaaaagaaagccgtgaaggagtcttctatccgatctgtgcaggagac
Agtactccccatcaagaagcgcaagacccgggagtaa.

In an aspect, a disclosed KRAB-MeCP2 repressor can comprise the sequence set forth below or a sequence having 70%, 75%, 80%, 85%, 90%, 95%, or more identity to the sequence set forth:

(SEQ ID NO: 63)
MRTLVTFKDVFVDFTREEWKLLDTAQQIVYRNVMLENYKNLVSLGYQLTK
PDVILRLEKGEEPSGGGSGGGSEGVQVKRVLEKSPGKLLVKMPFQTSPGG
KAEGGGATTSTQVMVIKRPGRKRKAEADPQAIPKKRGRKPGSVVAAAAAE
AKKKAVKESSIRSVQETVLPIKKRKTRE*.

In an aspect, a disclosed gRNA can be designed to target exon 4 of the APOE gene or designed to target a protospacer-adjacent motif (PAM) created by a SNP rs429358 in exon 4 of the APOE gene. In an aspect, a disclosed gRNA can have the sequence set forth in any one of SEQ ID NO:05-SEQ ID NO:14, SEQ ID NO:25-SEQ ID NO:28, SEQ ID NO:39-SEQ ID NO:42, and SEQ ID NO:51-SEQ ID NO:52.

In an aspect, a disclosed Cas endonuclease can be fused to a disclosed polypeptide having an enzymatic activity. In an aspect, a disclosed Cas endonuclease can be dCas9 and a disclosed polypeptide can be DNMT3A. In an aspect, a disclosed dCas9-DNMT3A fusion protein can have the amino acid sequence set forth SEQ ID NO: 19. In an aspect, a dCas9-DNMT3A fusion protein can be encoded by the sequence set forth in SEQ ID NO:20. In an aspect, a disclosed dCas9-DNMT3A fusion protein can have a sequence having at least 75%, at least 80%, at least 85%, at least 90%, or at least 95% identity to the sequence set forth in SEQ ID NO:19 or SEQ ID NO:20 or a fragment thereof. In an aspect, a disclosed Cas endonuclease can be dVRER and the polypeptide can be DNMT3A. In an aspect, a disclosed dVRER-DNMT3A fusion protein can have the amino acid sequence set forth SEQ ID NO:38. In an aspect, a disclosed dVRER-DNMT3A fusion protein can be encoded by the sequence set forth in SEQ ID NO:37. In an aspect, a disclosed dVRER-DNMT3A fusion protein can have a sequence having at least 75%, at least 80%, at least 85%, at least 90%, or at least 95% identity to the sequence set forth in SEQ ID NO:38 or SEQ ID NO:37 or a fragment thereof.

In an aspect, a disclosed Cas endonuclease can be dCas9 and the polypeptide can be Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed Cas endonuclease can be dVRER and the polypeptide can be Krüppel-associated box (KRAB), the transcription repression domain (TRD) of Methyl-CpG Binding Protein 2 (MeCP2), or a fusion of KRAB-MeCP2 (KRAB-MeCP2).

In an aspect, a disclosed dCas9-DNMT3A fusion protein can comprise the sequence set forth below or a sequence having 70%, 75%, 80%, 85%, 90%, 95%, or more identity to the sequence set forth:

(SEQ ID NO: 19)
DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGDKRPAATKKAGQAKKKKLEGGGGSGSPSRLQMFF
ANNHDQEFDPPKVYPPVPAEKRKPIRVLSLFDGIATGLLVLKDLGIQVDR
YIASEVCEDSITVGMVRHQGKIMYVGDVRSVTQKHIQEWGPFDLVIGGSP
CNDLSIVNPARKGLYEGTGRLFFEFYRLLHDARPKEGDDRPFFWLFENVV
AMGVSDKRDISRFLESNPVMIDAKEVSAAHRARYFWGNLPGMNRPLASTV
NDKLELQECLEHGRIAKFSKVRTITTRSNSIKQGKDQHFPVFMNEKEDIL
WCTEMERVFGFPVHYTDVSNMSRLARQRLLGRSWSVPVIRHLFAPLKEYF
AC.

In an aspect, a disclosed dCas9-DNMT3A fusion protein can comprise the sequence set forth below or a sequence having 70%, 75%, 80%, 85%, 90%, 95%, or more identity to the sequence set forth:

(SEQ ID NO: 20)
gacaagaagtacagcatcggcctggccatcggcaccaactctgtgggctgggccgtgatcaccgacgagtacaaggtgcccagcaaga
aattcaaggtgctgggcaacaccgaccggcacagcatcaagaagaacctgatcggagccctgctgttcgacagcggcgaaacagccga
ggccacccggctgaagagaaccgccagaagaagatacaccagacggaagaaccggatctgctatctgcaagagatcttcagcaacgag
atggccaaggtggacgacagcttcttccacagactggaagagtccttcctggtggaagaggataagaagcacgagcggcaccccatcttc
ggcaacatcgtggacgaggtggcctaccacgagaagtaccccaccattaccacctgagaaagaaactggtggacagcaccgacaagg
ccgacctgcggctgatctatctggccctggcccacatgatcaagttccggggccacttcctgatcgagggcgacctgaaccccgacaaca
gcgacgtggacaagctgttcatccagctggtgcagacctacaaccagctgttcgaggaaaaccccatcaacgccagcggcgtggacgcc
aaggccatcctgtctgccagactgagcaagagcagacggctggaaaatctgatcgcccagctgcccggcgagaagaagaatggcctgtt
cggcaacctgattgccctgagcctgggcctgacccccaacttcaagagcaacttcgacctggccgaggatgccaaactgcagctgagca
aggacacctacgacgacgacctggacaacctgctggcccagateggcgaccagtacgccgacctgtttctggccgccaagaacctgtcc
gacgccatcctgctgagcgacatcctgagagtgaacaccgagatcaccaaggcccccctgagcgcctctatgatcaagagatacgacga
gcaccaccaggacctgaccctgctgaaagctctcgtgcggcagcagctgcctgagaagtacaaagagattttcttcgaccagagcaagaa
cggctacgccggctacattgacggcggagccagccaggaagagttctacaagttcatcaagcccatcctggaaaagatggacggcaccg
aggaactgctcgtgaagctgaacagagaggacctgctgcggaagcageggaccttegacaacggcagcatcccccaccagatccacct
gggagagctgcacgccattctgcggcggcaggaagatttttacccattcctgaaggacaaccgggaaaagatcgagaagatcctgacctt
ccgcatcccctactacgtgggccctctggccaggggaaacagcagattcgcctggatgaccagaaagagegaggaaaccatcaccccct
ggaacttcgaggaagtggtggacaagggcgcttccgcccagagcttcatcgagcggatgaccaacttcgataagaacctgcccaacgag
aaggtgctgcccaagcacagcctgctgtacgagtacttcaccgtgtataacgagctgaccaaagtgaaatacgtgaccgagggaatgaga
aagcccgccttcctgagcggcgagcagaaaaaggccatcgtggacctgctgttcaagaccaaccggaaagtgaccgtgaagcagctga
aagaggactacttcaagaaaatcgagtgcttcgactccgtggaaatctccggcgtggaagatcggttcaacgcctccctgggcacatacca
cgatctgctgaaaattatcaaggacaaggacttcctggacaatgaggaaaacgaggacattctggaagatatcgtgctgaccctgacactgt
ttgaggacagagagatgatcgaggaacggctgaaaacctatgcccacctgttcgacgacaaagtgatgaagcagctgaagcggcggag
atacaccggctggggcaggctgagccggaagctgatcaacggcatccgggacaagcagtccggcaagacaatcctggatttcctgaagt
ccgacggcttcgccaacagaaacttcatgcagctgatccacgacgacagcctgacctttaaagaggacatccagaaagcccaggtgtccg
gccagggcgatagcctgcacgagcacattgccaatctggccggcagccccgccattaagaagggcatcctgcagacagtgaaggtggt
ggacgagctcgtgaaagtgatgggccggcacaagcccgagaacatcgtgatcgaaatggccagagagaaccagaccacccagaagg
gacagaagaacagccgcgagagaatgaagcggatcgaagagggcatcaaagagctgggcagccagatcctgaaagaacaccccgtg
gaaaacacccagctgcagaacgagaagctgtacctgtactacctgcagaatggggggatatgtacgtggaccaggaactggacatcaa
ccggctgtccgactacgatgtggacgctatcgtgcctcagagctttctgaaggacgactccatcgacaacaaggtgctgaccagaagcga
caagaaccggggcaagagcgacaacgtgccctccgaagaggtcgtgaagaagatgaagaactactggcggcagctgctgaacgccaa
gctgattacccagagaaagttcgacaatctgaccaaggccgagagaggcggcctgagcgaactggataaggccggcttcatcaagaga
cagctggtggaaacccggcagatcacaaagcacgtggcacagatcctggactcccggatgaacactaagtacgacgagaatgacaagc
tgatccgggaagtgaaagtgatcaccctgaagtccaagctggtgtccgatttccggaaggatttccagttttacaaagtgcgcgagatcaac
aactaccaccacgcccacgacgcctacctgaacgccgtcgtgggaaccgccctgatcaaaaagtaccctaagctggaaagcgagttcgt
gtacggcgactacaaggtgtacgacgtgcggaagatgatcgccaagagcgagcaggaaatcggcaaggctaccgccaagtacttcttct
acagcaacatcatgaactttttcaagaccgagattaccctggccaacggcgagatccggaagcggcctctgatcgagacaaacggcgaa
accggggagatcgtgtgggataagggccgggattttgccaccgtgcggaaagtgctgagcatgccccaagtgaatatcgtgaaaaagac
cgaggtgcagacaggcggcttcagcaaagagtctatcctgcccaagaggaacagcgataagctgatcgccagaaagaaggactgggac
cctaagaagtacggcggcttcgacagccccaccgtggcctattctgtgctggtggtggccaaagtggaaaagggcaagtccaagaaact
gaagagtgtgaaagagctgctggggatcaccatcatggaaagaagcagcttcgagaagaatcccatcgactttctggaagccaagggcta
caaagaagtgaaaaaggacctgatcatcaagctgcctaagtactccctgttcgagctggaaaacggccggaagagaatgctggcctctgc
cggcgaactgcagaagggaaacgaactggccctgccctccaaatatgtgaacttcctgtacctggccagccactatgagaagctgaagg
gctcccccgaggataatgagcagaaacagctgtttgtggaacagcacaagcactacctggacgagatcatcgagcagatcagcgagttct
ccaagagagtgatcctggccgacgctaatctggacaaagtgctgtccgcctacaacaagcaccgggataagcccatcagagagcaggc
cgagaatatcatccacctgtttaccctgaccaatctgggagcccctgccgccttcaagtactttgacaccaccatcgaccggaagaggtaca
ccagcaccaaagaggtgctggacgccaccctgatccaccagagcatcaccggcctgtacgagacacggatcgacctgtctcagctggg
aggcgacaaaaggccggcggccacgaaaaaggccggacaggccaaaaagaaaaagctcgagggcggaggcgggagcggatcccc
ctcccggctccagatgttcttcgctaataaccacgaccaggaatttgaccctccaaaggtttacccacctgtcccagctgagaagaggaagc
ccatccgggtgctgtctctctttgatggaatcgctacagggctcctggtgctgaaggacttgggcattcaggtggaccgctacattgcctcgg
aggtgtgtgaggactccatcacggtgggcatggtgcggcaccaggggaagatcatgtacgtcggggacgtccgcagcgtcacacagaa
gcatatccaggagtggggcccattcgatctggtgattgggggcagtccctgcaatgacctctccatcgtcaaccctgctcgcaagggcctct
acgagggcactggccggctcttctttgagttctaccgcctcctgcatgatgcgcggcccaaggagggagatgatcgccccttcttctggctc
tttgagaatgtggtggccatgggcgttagtgacaagagggacatctcgcgatttctcgagtccaaccctgtgatgattgatgccaaagaagt
gtcagctgcacacagggcccgctacttctggggtaaccttcccggtatgaacaggccgttggcatccactgtgaatgataagctggagctg
caggagtgtctggagcatggcaggatagccaagttcagcaaagtgaggaccattactacgaggtcaaactccataaagcagggcaaaga
ccagcattttcctgtgttcatgaatgagaaagaggacatcttatggtgcactgaaatggaaagggtatttggtttcccagtccactatactg
acgtgtccaacatgagccgcttggcgaggcagagactgctgggccggtcatggagcgtgccagtcatccgccacctcttcgctccgctgaag
gagtattttgcgtgtgtg.

In an aspect, a disclosed SpCas9-dVRER-DNMT3A can comprise the sequence set forth below or a sequence having 70%, 75%, 80%, 85%, 90%, 95%, or more identity to the sequence set forth below:

(SEQ ID NO: 37)
atggactataaggaccacgacggagactacaaggatcatgatattgattacaaagacgatgacgataagatggccccaaagaagaagcg
gaaggtcggtatccacggagtcccagcagccgacaagaagtacagcatcggcctggccatcggcaccaactctgtgggctgggccgtg
atcaccgacgagtacaaggtgcccagcaagaaattcaaggtgctgggcaacaccgaccggcacagcatcaagaagaacctgatcggag
ccctgctgttcgacagcggcgaaacagccgaggccacccggctgaagagaaccgccagaagaagatacaccagacggaagaaccgg
atctgctatctgcaagagatcttcagcaacgagatggccaaggtggacgacagcttcttccacagactggaagagtccttcctggtggaag
aggataagaagcacgagcggcaccccatcttcggcaacatcgtggacgaggtggcctaccacgagaagtaccccaccatctaccacctg
agaaagaaactggtggacagcaccgacaaggccgacctgcggctgatctatctggccctggcccacatgatcaagttccggggccactt
cctgatcgagggcgacctgaaccccgacaacagcgacgtggacaagctgttcatccagctggtgcagacctacaaccagctgttcgagg
aaaaccccatcaacgccagcggcgtggacgccaaggccatcctgtctgccagactgagcaagagcagacggctggaaaatctgatcgc
ccagctgcccggcgagaagaagaatggcctgttcggcaacctgattgccctgagcctgggcctgacccccaacttcaagagcaacttcga
cctggccgaggatgccaaactgcagctgagcaaggacacctacgacgacgacctggacaacctgctggcccagateggcgaccagtac
gccgacctgtttctggccgccaagaacctgtccgacgccatcctgctgagcgacatcctgagagtgaacaccgagatcaccaaggcccc
cctgagcgcctctatgatcaagagatacgacgagcaccaccaggacctgaccctgctgaaagctctcgtgcggcagcagctgcctgaga
agtacaaagagattttcttcgaccagagcaagaacggctacgccggctacattgacggcggagccagccaggaagagttctacaagttca
tcaagcccatcctggaaaagatggacggcaccgaggaactgctcgtgaagctgaacagagaggacctgctgcggaagcagcggacctt
cgacaacggcagcatcccccaccagatccacctgggagagctgcacgccattctgcggcggcaggaagatttttacccattcctgaagga
caaccgggaaaagatcgagaagatcctgaccttccgcatcccctactacgtgggccctctggccaggggaaacagcagattcgcctggat
gaccagaaagagcgaggaaaccatcaccccctggaacttcgaggaagtggtggacaagggcgcttccgcccagagcttcatcgagcgg
atgaccaacttcgataagaacctgcccaacgagaaggtgctgcccaagcacagcctgctgtacgagtacttcaccgtgtataacgagctga
ccaaagtgaaatacgtgaccgagggaatgagaaagcccgccttcctgagcggcgagcagaaaaaggccatcgtggacctgctgttcaa
gaccaaccggaaagtgaccgtgaagcagctgaaagaggactacttcaagaaaatcgagtgcttcgactccgtggaaatctccggcgtgg
aagatcggttcaacgcctccctgggcacataccacgatctgctgaaaattatcaaggacaaggacttcctggacaatgaggaaaacgagg
acattctggaagatatcgtgctgaccctgacactgtttgaggacagagagatgatcgaggaacggctgaaaacctatgcccacctgttcga
cgacaaagtgatgaagcagctgaagcggcggagatacaccggctggggcaggctgagccggaagctgatcaacggcatccgggaca
agcagtccggcaagacaatcctggatttcctgaagtccgacggcttcgccaacagaaacttcatgcagctgatccacgacgacagcctga
cctttaaagaggacatccagaaagcccaggtgtccggccagggcgatagcctgcacgagcacattgccaatctggccggcagccccgc
cattaagaagggcatcctgcagacagtgaaggtggtggacgagctcgtgaaagtgatgggccggcacaagcccgagaacatcgtgatc
gaaatggccagagagaaccagaccacccagaagggacagaagaacagccgcgagagaatgaagcggatcgaagagggcatcaaag
agctgggcagccagatcctgaaagaacaccccgtggaaaacacccagctgcagaacgagaagctgtacctgtactacctgcagaatgg
gcgggatatgtacgtggaccaggaactggacatcaaccggctgtccgactacgatgtggacgctatcgtgcctcagagctttctgaaggac
gactccatcgacaacaaggtgctgaccagaagcgacaagaaccggggcaagagcgacaacgtgccctccgaagaggtcgtgaagaag
atgaagaactactggcggcagctgctgaacgccaagctgattacccagagaaagttcgacaatctgaccaaggccgagagaggcggcct
gagcgaactggataaggccggcttcatcaagagacagctggtggaaacccggcagatcacaaagcacgtggcacagatactagattccc
gaatgaatacgaaatacgacgagaacgataagctgattcgggaagtcaaagtaatcactttaaagtcaaaattggtgtcggacttcagaaagg
attttcaattctataaagttagggagataaataactaccaccatgcgcacgacgcttatcttaatgccgtcgtagggaccgcactcattaaga
aatacccgaagctagaaagtgagtttgtgtatggtgattacaaagtttatgacgtccgtaagatgatcgcgaaaagcgaacaggagataggc
aaggctacagccaaatacttcttttattctaacattatgaatttctttaagacggaaatcactctggcaaacggagagatacgcaaacgacct
ttaattgaaaccaatggggagacaggtgaaatcgtatgggataagggccgggacttcgcgacggtgagaaaagttttgtccatgccccaagt
caacatagtaaagaaaactgaggtgcagaccggagggttttcaaaggaatcgattcttccaaaaaggaatagtgataagctcatcgctcgta
aaaaggactgggacccgaaaaagtacggtggcttcgtgagccctacagttgcctattctgtcctagtagtggcaaaagttgagaagggaaa
atccaagaaactgaagtcagtcaaagaattattggggataacgattatggagcgctcgtcttttgaaaagaaccccatcgacttccttgaggc
gaaaggttacaaggaagtaaaaaaggatctcataattaaactaccaaagtatagtctgtttgagttagaaaatggccgaaaacggatgttgg
ctagcgccagagagcttcaaaaggggaacgaactcgcactaccgtctaaatacgtgaatttcctgtatttagcgtcccattacgagaagttga
aaggttcacctgaagataacgaacagaagcaactttttgttgagcagcacaaacattatctcgacgaaatcatagagcaaatttcggaattca
gtaagagagtcatcctagctgatgccaatctggacaaagtattaagcgcatacaacaagcacagggataaacccatacgtgagcaggcgg
aaaatattatccatttgtttactcttaccaacctcggcgctccagccgcattcaagtattttgacacaacgatagatcgcaaagagtacagat
ctaccaaggaggtgctagacgcgacactgattcaccaatccatcacgggattatatgaaactcggatagatttgtcacagcttgggggtgacg
gatcccccaagaagaagaggaaagtcctcgagggcggaggcgggagcggatccccctcccggctccagatgttcttcgctaataaccac
gaccaggaatttgaccctccaaaggtttacccacctgtcccagctgagaagaggaagcccatccgggtgctgtctctctttgatggaatcgc
tacagggctcctggtgctgaaggacttgggcattcaggtggaccgctacattgcctcggaggtgtgtgaggactccatcacggtgggcatg
gtgcggcaccaggggaagatcatgtacgtcggggacgtccgcagcgtcacacagaagcatatccaggagtggggcccattcgatctggt
gattgggggcagtccctgcaatgacctctccatcgtcaaccctgctcgcaagggcctctacgagggcactggccggctcttctttgagttct
accgcctcctgcatgatgcgcggcccaaggagggagatgatcgccccttcttctggctctttgagaatgtggtggccatgggcgttagtga
caagagggacatctcgcgatttctcgagtccaaccctgtgatgattgatgccaaagaagtgtcagctgcacacagggcccgctacttctgg
ggtaaccttcccggtatgaacaggccgttggcatccactgtgaatgataagctggagctgcaggagtgtctggagcatggcaggatagcc
aagttcagcaaagtgaggaccattactacgaggtcaaactccataaagcagggcaaagaccagcattttcctgtgttcatgaatgagaaag
aggacatcttatggtgcactgaaatggaaagggtatttggtttcccagtccactatactgacgtgtccaacatgagccgcttggcgaggcag
agactgctgggccggtcatggagcgtgccagtcatccgccacctcttcgctccgctgaaggagtattttgcgtgtgtg.

In an aspect, a disclosed VRER-DNMT3A can comprise the sequence set forth below or a sequence having 70%, 75%, 80%, 85%, 90%, 95%, or more identity to the sequence set forth below:

(SEQ ID NO: 38)
MAPKKKRKVGIHGVPAADKKYSIGLAIGTNSVGWAVITDEYKVPSKKFKV
LGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLK
SDGFANRNFMQLIHDDSLTFKEDIQKAQVSGQGDSLHEHANLAGSPAIKK
GILQTVKVVDELVKVMGRHKPENIVIEMARENQTTQKGQKNSRERMKRIE
EGIKELGSQILKEHPVENTQLQNEKLYLYYLONGRDMYVDQELDINRLSD
YDVDAIVPQSFLKDDSIDNKVLTRSDKNRGKSDNVPSEEVVKKMKNYWRQ
LLNAKLITQRKFDNLTKAERGGLSELDKAGFIKRQLVETRQITKHVAQIL
DSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAK
YFFYSNIMNFFKTEITLAGEIRKRPLIETNGETGEIVWDKGRDFATVRKV
LSMPQVNIVKKTEVQTGGFSKESILPKRNSDKLIARKKDWDPKKYGGFVS
PTVAYSVLVVAKVEKGKSKKLKSVKELLGITIMERSSFEKNPIDFLEAKG
YKEVKKDLIIKLPKYSLFELENGRKRMLASARELQKGNLKGSPEDNEQKQ
LFVEQHKHYLDEIIEQISEFSKRVILADANLDKVLSAYNKHRDKPIREQA
ENIIHLFTLTNLGAPAAFKYFDTTIDRKEYRSTKEVLDATLIHQSITGLY
ETRIDLSQLGGDGSPKKKVLEGGGGSGSPSRLQMFFANNHDQEFDPPKVY
PPVPAEKRKPIRVLSLFDGIATGLLVLKDLGIQVDRYIASEVCEDSITVG
MVRHQGKIMYVGDVRSVTQKHIQEWGPFDLVIGGSPCNDLSIVNPARKGL
YEGTGRLFFEFYRLLHDARPKEGDDRPFFWLFENVVAMGVSDKRDISRFL
ESNPVMIDAKEVSAAHRARYFWGNLPGMNRPLASTVNDKLELQECLEHRI
AKFSKVRTITTRSNSIKQGKDQHFPVFMNEKEDILWCTEMERVFGFPVHY
TDVSNMSRLARQRLLGRSWSVPVIRHLFAPLKEYFACV.

Disclosed herein is an isolated nucleic acid molecule comprising a nucleic acid sequence encoding (i) a fusion protein and (ii) at least one guide RNA.

In an aspect, a disclosed fusion protein can encode a disclosed Cas endonuclease and a disclosed polypeptide.

In an aspect, a disclosed Cas endonuclease can comprise Cas9, SpCas9, SaCas9, a variant Cas9, a dCas, or a dCas9. In an aspect, a disclosed Cas9 can comprise the sequence set forth in SEQ ID NO:64 or SEQ ID NO:65. In an aspect, a disclosed Cas9 can have a sequence having at least 75%, at least 80%, at least 85%, at least 90%, or at least 95% identity to the sequence set forth in SEQ ID NO:64 or SEQ ID NO:65 or a fragment thereof.

In an aspect, a disclosed variant Cas9 can comprise VQR, EQR, or VRER. In an aspect a disclosed VRER can comprise the sequence set forth in SEQ ID NO:15. In an aspect, a disclosed VRER can have a sequence having at least 75%, at least 80%, at least 85%, at least 90%, or at least 95% identity to the sequence set forth in SEQ ID NO:15 or a fragment thereof. In an aspect, a disclosed dCas can comprise dVQR, dEQR, or dVRER. In an aspect, a disclosed dCas can comprise the sequence set forth in SEQ ID NO:16. In an aspect, a disclosed dCas9 can have a sequence having at least 75%, at least 80%, at least 85%, at least 90%, or at least 95% identity to the sequence set forth in SEQ ID NO:16 or a fragment thereof. A SpCas9 (3′NGG-PAM sequence) can comprise SpCas9 VQR (3′NGAN or 3′NGNG), SpCas9 EQR (3′NGAG), or SpCas9 VRER (3′NGCG).

In an aspect, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof. In an aspect, a disclosed encoded polypeptide can be histone deacetylase or heterochromatin protein 1. In an aspect, a disclosed encoded polypeptide can comprise transcription repression activity. In an aspect, a disclosed DNMT3A can have the amino acid sequence set forth in SEQ ID NO:17 or the nucleotide sequence set forth in SEQ ID NO:18. In an aspect, a disclosed DNMT3A can have a sequence having at least 75%, at least 80%, at least 85%, at least 90%, or at least 95% identity to the sequence set forth in SEQ ID NO:17 or SEQ ID NO:18 or a fragment thereof.

In an aspect, at least one encoded polypeptide can comprise Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed TRD of MeCP2 can comprise the nucleotide sequence set forth in SEQ ID NO:57 or the amino acid sequence set forth in SEQ ID NO:58. In an aspect, a disclosed TRD of MeCP2 can have a sequence having at least 75%, at least 80%, at least 85%, at least 90%, or at least 95% identity to the sequence set forth in SEQ ID NO:57 or SEQ ID NO:58 or a fragment thereof. In an aspect, a disclosed KRAB-MeCP2 can comprise the nucleotide sequence set forth in SEQ ID NO:62 or the amino acid sequence set forth in SEQ ID NO:63. In an aspect, a disclosed KRAB-MeCP2 can have a sequence having at least 75%, at least 80%, at least 85%, at least 90%, or at least 95% identity to the sequence set forth in SEQ ID NO:62 or SEQ ID NO:63 or a fragment thereof.

In an aspect, a disclosed gRNA can be designed to target exon 4 of the APOE gene or designed to target a protospacer-adjacent motif (PAM) created by a SNP rs429358 in exon 4 of the APOE gene. In an aspect, a disclosed gRNA can have the sequence set forth in any one of SEQ ID NO:05-SEQ ID NO:14, SEQ ID NO:25-SEQ ID NO:28, SEQ ID NO:39-SEQ ID NO:42, and SEQ ID NO:51-SEQ ID NO:52.

In an aspect, a disclosed fusion protein can comprise dCas9 and DNMT3A. In an aspect, a dCas9-DNMT3A fusion protein can have the amino acid sequence set forth SEQ ID NO:19. In an aspect, a dCas9-DNMT3A fusion protein can be encoded by the sequence set forth in SEQ ID NO:20. In an aspect, a disclosed dCas9-DNMT3A fusion protein can have a sequence having at least 75%, at least 80%, at least 85%, at least 90%, or at least 95% identity to the sequence set forth in SEQ ID NO:19 or SEQ ID NO:20 or a fragment thereof. In an aspect, a disclosed Cas endonuclease can be dVRER and the polypeptide can be DNMT3A. In an aspect, a disclosed dVRER-DNMT3A fusion protein can have the amino acid sequence set forth SEQ ID NO:38. In an aspect, a disclosed dVRER-DNMT3A fusion protein can be encoded by the sequence set forth in SEQ ID NO:37. In an aspect, a disclosed dVRER-DNMT3A fusion protein can have a sequence having at least 75%, at least 80%, at least 85%, at least 90%, or at least 95% identity to the sequence set forth in SEQ ID NO:38 or SEQ ID NO:37 or a fragment thereof.

In an aspect, a disclosed fusion protein can comprise dCas9 and Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed fusion protein can comprise dVRER and Krüppel-associated box (KRAB), the transcription repression domain (TRD) of Methyl-CpG Binding Protein 2 (MeCP2), or a fusion of KRAB-MeCP2 (KRAB-MeCP2).

### 2. Non-Viral Vectors

Disclosed herein is a non-viral vector comprising a disclosed isolated nucleic acid molecule.

Disclosed herein is a non-viral vector comprising a disclosed isolated nucleic acid molecule comprising a nucleic acid sequence encoding (i) a Cas endonuclease, (ii) at least one polypeptide having an enzymatic activity, and (iii) at least one guide RNA.

Disclosed herein is a non-viral vector comprising a disclosed isolated nucleic acid molecule comprising a nucleic acid sequence encoding (i) a fusion protein and (ii) at least one guide RNA.

In an aspect, a disclosed non-viral vector can be a polymer based vector, a peptide based vector, a lipid nanoparticle, a solid lipid nanoparticle, or a cationic lipid based vector.

In an aspect, a disclosed non-viral vector can comprise one or more promoters operably linked to the isolated nucleic acid molecule. In an aspect, a disclosed promoter can drive the expression of a gRNA, the Cas9 endonuclease, a polypeptide, or a combination thereof. In an aspect, a disclosed promoter can be a hU6 promoter and a disclosed hU6 promoter can drive expression of a gRNA. In an aspect, a promoter can be an EFS-NC promoter and a disclosed EFS-NC promoter can drive expression of the Cas endonuclease. In an aspect, a disclosed promoter can comprise a hU6 promoter, an EFS-NC promoter, or a combination thereof.

In an aspect, a disclosed Cas endonuclease can comprise Cas9, SpCas9, SaCas9, a variant Cas9, a dCas, or a dCas9. In an aspect, a disclosed Cas9 can comprise the sequence set forth in SEQ ID NO:64 or SEQ ID NO:65. In an aspect, a disclosed Cas9 can have a sequence having at least 75%, at least 80%, at least 85%, at least 90%, or at least 95% identity to the sequence set forth in SEQ ID NO:64 or SEQ ID NO:65 or a fragment thereof.

In an aspect, a disclosed variant Cas9 can comprise VQR, EQR, or VRER. In an aspect a disclosed VRER can comprise the sequence set forth in SEQ ID NO:15. In an aspect, a disclosed VRER can have a sequence having at least 75%, at least 80%, at least 85%, at least 90%, or at least 95% identity to the sequence set forth in SEQ ID NO:15 or a fragment thereof. In an aspect, a disclosed dCas can comprise dVQR, dEQR, or dVRER. In an aspect, a disclosed dCas can comprise the sequence set forth in SEQ ID NO:16. In an aspect, a disclosed dCas9 can have a sequence having at least 75%, at least 80%, at least 85%, at least 90%, or at least 95% identity to the sequence set forth in SEQ ID NO:16 or a fragment thereof. A SpCas9 (3′NGG-PAM sequence) can comprise SpCas9 VQR (3′NGAN or 3′NGNG), SpCas9 EQR (3′NGAG), or SpCas9 VRER (3′NGCG).

In an aspect, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof. In an aspect, a disclosed encoded polypeptide can be histone deacetylase or heterochromatin protein 1. In an aspect, a disclosed encoded polypeptide can comprise transcription repression activity. In an aspect, a disclosed DNMT3A can have the amino acid sequence set forth in SEQ ID NO:17 or the nucleotide sequence set forth in SEQ ID NO:18. In an aspect, a disclosed DNMT3A can have a sequence having at least 75%, at least 80%, at least 85%, at least 90%, or at least 95% identity to the sequence set forth in SEQ ID NO:17 or SEQ ID NO:18 or a fragment thereof.

In an aspect, at least one encoded polypeptide can comprise Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed TRD of MeCP2 can comprise the nucleotide sequence set forth in SEQ ID NO:57 or the amino acid sequence set forth in SEQ ID NO:58. In an aspect, a disclosed TRD of MeCP2 can have a sequence having at least 75%, at least 80%, at least 85%, at least 90%, or at least 95% identity to the sequence set forth in SEQ ID NO:57 or SEQ ID NO:58 or a fragment thereof. In an aspect, a disclosed KRAB-MeCP2 can comprise the nucleotide sequence set forth in SEQ ID NO:62 or the amino acid sequence set forth in SEQ ID NO:63. In an aspect, a disclosed KRAB-MeCP2 can have a sequence having at least 75%, at least 80%, at least 85%, at least 90%, or at least 95% identity to the sequence set forth in SEQ ID NO:62 or SEQ ID NO:63 or a fragment thereof.

In an aspect, a disclosed gRNA can be designed to target exon 4 of the APOE gene or designed to target a protospacer-adjacent motif (PAM) created by a SNP rs429358 in exon 4 of the APOE gene. In an aspect, a disclosed gRNA can have the sequence set forth in any one of SEQ ID NO:05-SEQ ID NO:14, SEQ ID NO:25-SEQ ID NO:28, SEQ ID NO:39-SEQ ID NO:42, and SEQ ID NO:51-SEQ ID NO:52.

In an aspect, a disclosed Cas endonuclease can be fused to a disclosed polypeptide having an enzymatic activity. In an aspect, a disclosed fusion protein can encode a disclosed Cas endonuclease and a disclosed polypeptide. In an aspect, a disclosed fusion protein can comprise dCas9 and DNMT3A. In an aspect, a dCas9-DNMT3A fusion protein can have the amino acid sequence set forth SEQ ID NO:19. In an aspect, a dCas9-DNMT3A fusion protein can be encoded by the sequence set forth in SEQ ID NO:20. In an aspect, a disclosed dCas9-DNMT3A fusion protein can have a sequence having at least 75%, at least 80%, at least 85%, at least 90%, or at least 95% identity to the sequence set forth in SEQ ID NO:19 or SEQ ID NO:20 or a fragment thereof. In an aspect, a disclosed Cas endonuclease can be dVRER and the polypeptide can be DNMT3A. In an aspect, a disclosed dVRER-DNMT3A fusion protein can have the amino acid sequence set forth SEQ ID NO:38. In an aspect, a disclosed dVRER-DNMT3A fusion protein can be encoded by the sequence set forth in SEQ ID NO:37. In an aspect, a disclosed dVRER-DNMT3A fusion protein can have a sequence having at least 75%, at least 80%, at least 85%, at least 90%, or at least 95% identity to the sequence set forth in SEQ ID NO:38 or SEQ ID NO:37 or a fragment thereof.

In an aspect, a disclosed fusion protein can comprise dCas9 and Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed fusion protein can comprise dVRER and Krüppel-associated box (KRAB), the transcription repression domain (TRD) of Methyl-CpG Binding Protein 2 (MeCP2), or a fusion of KRAB-MeCP2 (KRAB-MeCP2).

In an aspect, a disclosed non-viral vector can comprise one or more regulatory elements. Regulatory elements are known in the art and can comprise one or more of the following: a Sp1 responsive element, a p2A signal, a woodchuck hepatitis virus post-transcriptional regulatory element, a Phi signal-packaging signal, a rev responsive element, a 5′-LTR, and a 3′-LTR. In an aspect, a disclosed non-viral vector can comprise two Sp1 response elements, a p2A signal, a woodchuck hepatitis virus post-transcriptional regulatory element, a Phi signal-packaging signal, a rev responsive element, a 5′-LTR, and a 3′-LTR.

### 3. Viral Vectors

Disclosed herein is a viral vector comprising a disclosed isolated nucleic acid molecule.

Disclosed herein is a viral vector comprising a disclosed isolated nucleic acid molecule comprising a nucleic acid sequence encoding (i) a Cas endonuclease, (ii) at least one polypeptide having an enzymatic activity, and (iii) at least one guide RNA.

In an aspect, a disclosed viral vector can comprise one or more promoters operably linked to the isolated nucleic acid molecule. In an aspect, a disclosed promoter can drive the expression of a gRNA, the Cas9 endonuclease, a polypeptide, or a combination thereof. In an aspect, a disclosed promoter can be a hU6 promoter and a disclosed hU6 promoter can drive expression of a gRNA. In an aspect, a promoter can be an EFS-NC promoter and a disclosed EFS-NC promoter can drive expression of the Cas endonuclease. In an aspect, a disclosed promoter can comprise a hU6 promoter, an EFS-NC promoter, or a combination thereof.

In an aspect, a disclosed Cas endonuclease can comprise Cas9, SpCas9, SaCas9, a variant Cas9, a dCas, or a dCas9. In an aspect, a disclosed Cas9 can comprise the sequence set forth in SEQ ID NO:64 or SEQ ID NO:65. In an aspect, a disclosed Cas9 can have a sequence having at least 75%, at least 80%, at least 85%, at least 90%, or at least 95% identity to the sequence set forth in SEQ ID NO:64 or SEQ ID NO:65 or a fragment thereof.

In an aspect, a disclosed variant Cas9 can comprise VQR, EQR, or VRER. In an aspect a disclosed VRER can comprise the sequence set forth in SEQ ID NO:15. In an aspect, a disclosed VRER can have a sequence having at least 75%, at least 80%, at least 85%, at least 90%, or at least 95% identity to the sequence set forth in SEQ ID NO:15 or a fragment thereof. In an aspect, a disclosed dCas can comprise dVQR, dEQR, or dVRER. In an aspect, a disclosed dCas can comprise the sequence set forth in SEQ ID NO:16. In an aspect, a disclosed dCas9 can have a sequence having at least 75%, at least 80%, at least 85%, at least 90%, or at least 95% identity to the sequence set forth in SEQ ID NO:16 or a fragment thereof. A SpCas9 (3′NGG-PAM sequence) can comprise SpCas9 VQR (3′NGAN or 3′NGNG), SpCas9 EQR (3′NGAG), or SpCas9 VRER (3′NGCG).

In an aspect, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof. In an aspect, a disclosed encoded polypeptide can be histone deacetylase or heterochromatin protein 1. In an aspect, a disclosed encoded polypeptide can comprise transcription repression activity. In an aspect, a disclosed DNMT3A can have the amino acid sequence set forth in SEQ ID NO:17 or the nucleotide sequence set forth in SEQ ID NO:18. In an aspect, a disclosed DNMT3A can have a sequence having at least 75%, at least 80%, at least 85%, at least 90%, or at least 95% identity to the sequence set forth in SEQ ID NO:17 or SEQ ID NO:18 or a fragment thereof.

In an aspect, at least one encoded polypeptide can comprise Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed TRD of MeCP2 can comprise the nucleotide sequence set forth in SEQ ID NO:57 or the amino acid sequence set forth in SEQ ID NO:58. In an aspect, a disclosed TRD of MeCP2 can have a sequence having at least 75%, at least 80%, at least 85%, at least 90%, or at least 95% identity to the sequence set forth in SEQ ID NO:57 or SEQ ID NO:58 or a fragment thereof. In an aspect, a disclosed KRAB-MeCP2 can comprise the nucleotide sequence set forth in SEQ ID NO:62 or the amino acid sequence set forth in SEQ ID NO:63. In an aspect, a disclosed KRAB-MeCP2 can have a sequence having at least 75%, at least 80%, at least 85%, at least 90%, or at least 95% identity to the sequence set forth in SEQ ID NO:62 or SEQ ID NO:63 or a fragment thereof.

In an aspect, a disclosed gRNA can be designed to target exon 4 of the APOE gene or designed to target a protospacer-adjacent motif (PAM) created by a SNP rs429358 in exon 4 of the APOE gene. In an aspect, a disclosed gRNA can have the sequence set forth in any one of SEQ ID NO:05-SEQ ID NO:14, SEQ ID NO:25-SEQ ID NO:28, SEQ ID NO:39-SEQ ID NO:42, and SEQ ID NO:51-SEQ ID NO:52.

In an aspect, a disclosed Cas endonuclease can be fused to a disclosed polypeptide having an enzymatic activity. In an aspect, a disclosed fusion protein can encode a disclosed Cas endonuclease and a disclosed polypeptide. In an aspect, a disclosed fusion protein can comprise dCas9 and DNMT3A. In an aspect, a dCas9-DNMT3A fusion protein can have the amino acid sequence set forth SEQ ID NO:19. In an aspect, a dCas9-DNMT3A fusion protein can be encoded by the sequence set forth in SEQ ID NO:20. In an aspect, a disclosed dCas9-DNMT3A fusion protein can have a sequence having at least 75%, at least 80%, at least 85%, at least 90%, or at least 95% identity to the sequence set forth in SEQ ID NO:19 or SEQ ID NO:20 or a fragment thereof. In an aspect, a disclosed Cas endonuclease can be dVRER and the polypeptide can be DNMT3A. In an aspect, a disclosed dVRER-DNMT3A fusion protein can have the amino acid sequence set forth SEQ ID NO:38. In an aspect, a disclosed dVRER-DNMT3A fusion protein can be encoded by the sequence set forth in SEQ ID NO:37. In an aspect, a disclosed dVRER-DNMT3A fusion protein can have a sequence having at least 75%, at least 80%, at least 85%, at least 90%, or at least 95% identity to the sequence set forth in SEQ ID NO:38 or SEQ ID NO:37 or a fragment thereof.

In an aspect, a disclosed fusion protein can comprise dCas9 and Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed fusion protein can comprise dVRER and Krüppel-associated box (KRAB), the transcription repression domain (TRD) of Methyl-CpG Binding Protein 2 (MeCP2), or a fusion of KRAB-MeCP2 (KRAB-MeCP2).

In an aspect, a disclosed viral vector can comprise one or more regulatory elements. Regulatory elements are known in the art and can comprise one or more of the following: a Sp1 responsive element, a p2A signal, a woodchuck hepatitis virus post-transcriptional regulatory element, a Phi signal-packaging signal, a rev responsive element, a 5′-LTR, and a 3′-LTR. In an aspect, a disclosed viral vector can comprise two Sp1 response elements, a p2A signal, a woodchuck hepatitis virus post-transcriptional regulatory element, a Phi signal-packaging signal, a rev responsive element, a 5′-LTR, and a 3′-LTR.

In an aspect, a disclosed viral vector can be an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector. In an aspect, a disclosed viral vector can be a lentiviral vector.

Disclosed herein is a viral vector comprising a disclosed isolated nucleic acid molecule comprising a nucleic acid sequence encoding (i) a fusion protein and (ii) at least one guide RNA.

In an aspect, a disclosed viral vector can comprise one or more promoters operably linked to the isolated nucleic acid molecule. In an aspect, a disclosed promoter can drive the expression of a gRNA, the Cas9 endonuclease, a polypeptide, or a combination thereof. In an aspect, a disclosed promoter can be a hU6 promoter and a disclosed hU6 promoter can drive expression of a gRNA. In an aspect, a promoter can be an EFS-NC promoter and a disclosed EFS-NC promoter can drive expression of the Cas endonuclease. In a aspect, a disclosed promoter can comprise a hU6 promoter, an EFS-NC promoter, or a combination thereof.

In an aspect, a disclosed Cas endonuclease can comprise Cas9, SpCas9, SaCas9, a variant Cas9, a dCas, or a dCas9. In an aspect, a disclosed Cas9 can comprise the sequence set forth in SEQ ID NO:64 or SEQ ID NO:65. In an aspect, a disclosed Cas9 can have a sequence having at least 75%, at least 80%, at least 85%, at least 90%, or at least 95% identity to the sequence set forth in SEQ ID NO:64 or SEQ ID NO:65 or a fragment thereof.

In an aspect, a disclosed variant Cas9 can comprise VQR, EQR, or VRER. In an aspect a disclosed VRER can comprise the sequence set forth in SEQ ID NO:15. In an aspect, a disclosed VRER can have a sequence having at least 75%, at least 80%, at least 85%, at least 90%, or at least 95% identity to the sequence set forth in SEQ ID NO:15 or a fragment thereof. In an aspect, a disclosed dCas can comprise dVQR, dEQR, or dVRER. In an aspect, a disclosed dCas can comprise the sequence set forth in SEQ ID NO:16. In an aspect, a disclosed dCas9 can have a sequence having at least 75%, at least 80%, at least 85%, at least 90%, or at least 95% identity to the sequence set forth in SEQ ID NO:16 or a fragment thereof. A SpCas9 (3′NGG-PAM sequence) can comprise SpCas9 VQR (3′NGAN or 3′NGNG), SpCas9 EQR (3′NGAG), or SpCas9 VRER (3′NGCG).

In an aspect, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof. In an aspect, a disclosed encoded polypeptide can be histone deacetylase or heterochromatin protein 1. In an aspect, a disclosed encoded polypeptide can comprise transcription repression activity. In an aspect, a disclosed DNMT3A can have the amino acid sequence set forth in SEQ ID NO:17 or the nucleotide sequence set forth in SEQ ID NO:18. In an aspect, a disclosed DNMT3A can have a sequence having at least 75%, at least 80%, at least 85%, at least 90%, or at least 95% identity to the sequence set forth in SEQ ID NO:17 or SEQ ID NO:18 or a fragment thereof.

In an aspect, at least one encoded polypeptide can comprise Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed TRD of MeCP2 can comprise the nucleotide sequence set forth in SEQ ID NO:57 or the amino acid sequence set forth in SEQ ID NO:58. In an aspect, a disclosed TRD of MeCP2 can have a sequence having at least 75%, at least 80%, at least 85%, at least 90%, or at least 95% identity to the sequence set forth in SEQ ID NO:57 or SEQ ID NO:58 or a fragment thereof. In an aspect, a disclosed KRAB-MeCP2 can comprise the nucleotide sequence set forth in SEQ ID NO:62 or the amino acid sequence set forth in SEQ ID NO:63. In an aspect, a disclosed KRAB-MeCP2 can have a sequence having at least 75%, at least 80%, at least 85%, at least 90%, or at least 95% identity to the sequence set forth in SEQ ID NO:62 or SEQ ID NO:63 or a fragment thereof.

In an aspect, a disclosed gRNA can be designed to target exon 4 of the APOE gene or designed to target a protospacer-adjacent motif (PAM) created by a SNP rs429358 in exon 4 of the APOE gene. In an aspect, a disclosed gRNA can have the sequence set forth in any one of SEQ ID NO:05-SEQ ID NO:14, SEQ ID NO:25-SEQ ID NO:28, SEQ ID NO:39-SEQ ID NO:42, and SEQ ID NO:51-SEQ ID NO:52.

In an aspect, a disclosed fusion protein can encode a disclosed Cas endonuclease and a disclosed polypeptide. In an aspect, a disclosed fusion protein can comprise dCas9 and DNMT3A. In an aspect, a dCas9-DNMT3A fusion protein can have the amino acid sequence set forth SEQ ID NO:19. In an aspect, a dCas9-DNMT3A fusion protein can be encoded by the sequence set forth in SEQ ID NO:20. In an aspect, a disclosed dCas9-DNMT3A fusion protein can have a sequence having at least 75%, at least 80%, at least 85%, at least 90%, or at least 95% identity to the sequence set forth in SEQ ID NO:19 or SEQ ID NO:20 or a fragment thereof. In an aspect, a disclosed Cas endonuclease can be dVRER and the polypeptide can be DNMT3A. In an aspect, a disclosed dVRER-DNMT3A fusion protein can have the amino acid sequence set forth SEQ ID NO:38. In an aspect, a disclosed dVRER-DNMT3A fusion protein can be encoded by the sequence set forth in SEQ ID NO:37. In an aspect, a disclosed dVRER-DNMT3A fusion protein can have a sequence having at least 75%, at least 80%, at least 85%, at least 90%, or at least 95% identity to the sequence set forth in SEQ ID NO:38 or SEQ ID NO:37 or a fragment thereof.

In an aspect, a disclosed fusion protein can comprise dCas9 and Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed fusion protein can comprise dVRER and Krüppel-associated box (KRAB), the transcription repression domain (TRD) of Methyl-CpG Binding Protein 2 (MeCP2), or a fusion of KRAB-MeCP2 (KRAB-MeCP2).

In an aspect, a disclosed viral vector can comprise one or more regulatory elements. Regulatory elements are known in the art and can comprise one or more of the following: a Sp1 responsive element, a p2A signal, a woodchuck hepatitis virus post-transcriptional regulatory element, a Phi signal-packaging signal, a rev responsive element, a 5′-LTR, and a 3′-LTR. In an aspect, a disclosed vector can comprise two Sp1 response elements, a p2A signal, a woodchuck hepatitis virus post-transcriptional regulatory element, a Phi signal-packaging signal, a rev responsive element, a 5′-LTR, and a 3′-LTR.

In an aspect, a disclosed viral vector can be a lentiviral vector.

### 4. Lentiviral Vectors

Disclosed herein is a lentiviral vector comprising an isolated nucleic acid molecule comprising a nucleic acid sequence encoding (i) a Cas endonuclease, (ii) at least one polypeptide having an enzymatic activity, and (iii) at least one guide RNA.

In an aspect, a disclosed lentiviral vector can comprise one or more promoters operably linked to the isolated nucleic acid molecule. In an aspect, a disclosed promoter can drive the expression of a gRNA, the Cas9 endonuclease, a polypeptide, or a combination thereof. In an aspect, a disclosed promoter can be a hU6 promoter and a disclosed hU6 promoter can drive expression of a gRNA. In an aspect, a promoter can be an EFS-NC promoter and a disclosed EFS-NC promoter can drive expression of the Cas endonuclease. In an aspect, a disclosed promoter can comprise a hU6 promoter, an EFS-NC promoter, or a combination thereof.

In an aspect, a disclosed Cas endonuclease can comprise Cas9, SpCas9, SaCas9, a variant Cas9, a dCas, or a dCas9. In an aspect, a disclosed Cas9 can comprise the sequence set forth in SEQ ID NO:64 or SEQ ID NO:65. In an aspect, a disclosed Cas9 can have a sequence having at least 75%, at least 80%, at least 85%, at least 90%, or at least 95% identity to the sequence set forth in SEQ ID NO:64 or SEQ ID NO:65 or a fragment thereof.

In an aspect, a disclosed variant Cas9 can comprise VQR, EQR, or VRER. In an aspect a disclosed VRER can comprise the sequence set forth in SEQ ID NO:15. In an aspect, a disclosed VRER can have a sequence having at least 75%, at least 80%, at least 85%, at least 90%, or at least 95% identity to the sequence set forth in SEQ ID NO:15 or a fragment thereof. In an aspect, a disclosed dCas can comprise dVQR, dEQR, or dVRER. In an aspect, a disclosed dCas can comprise the sequence set forth in SEQ ID NO:16. In an aspect, a disclosed dCas9 can have a sequence having at least 75%, at least 80%, at least 85%, at least 90%, or at least 95% identity to the sequence set forth in SEQ ID NO:16 or a fragment thereof. A SpCas9 (3′NGG-PAM sequence) can comprise SpCas9 VQR (3′NGAN or 3′NGNG), SpCas9 EQR (3′NGAG), or SpCas9 VRER (3′NGCG).

In an aspect, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof. In an aspect, a disclosed encoded polypeptide can be histone deacetylase or heterochromatin protein 1. In an aspect, a disclosed encoded polypeptide can comprise transcription repression activity. In an aspect, a disclosed DNMT3A can have the amino acid sequence set forth in SEQ ID NO: 17 or the nucleotide sequence set forth in SEQ ID NO:18. In an aspect, a disclosed DNMT3A can have a sequence having at least 75%, at least 80%, at least 85%, at least 90%, or at least 95% identity to the sequence set forth in SEQ ID NO:17 or SEQ ID NO:18 or a fragment thereof.

In an aspect, at least one encoded polypeptide can comprise Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed TRD of MeCP2 can comprise the nucleotide sequence set forth in SEQ ID NO:57 or the amino acid sequence set forth in SEQ ID NO:58. In an aspect, a disclosed TRD of MeCP2 can have a sequence having at least 75%, at least 80%, at least 85%, at least 90%, or at least 95% identity to the sequence set forth in SEQ ID NO:57 or SEQ ID NO:58 or a fragment thereof. In an aspect, a disclosed KRAB-MeCP2 can comprise the nucleotide sequence set forth in SEQ ID NO:62 or the amino acid sequence set forth in SEQ ID NO:63. In an aspect, a disclosed KRAB-MeCP2 can have a sequence having at least 75%, at least 80%, at least 85%, at least 90%, or at least 95% identity to the sequence set forth in SEQ ID NO:62 or SEQ ID NO:63 or a fragment thereof.

In an aspect, a disclosed gRNA can be designed to target exon 4 of the APOE gene or designed to target a protospacer-adjacent motif (PAM) created by a SNP rs429358 in exon 4 of the APOE gene. In an aspect, a disclosed gRNA can have the sequence set forth in any one of SEQ ID NO:05-SEQ ID NO:14, SEQ ID NO:25-SEQ ID NO:28, SEQ ID NO:39-SEQ ID NO:42, and SEQ ID NO:51-SEQ ID NO:52.

In an aspect, a disclosed fusion protein can encode a disclosed Cas endonuclease and a disclosed polypeptide. In an aspect, a disclosed fusion protein can comprise dCas9 and DNMT3A. In an aspect, a dCas9-DNMT3A fusion protein can have the amino acid sequence set forth SEQ ID NO:19. In an aspect, a dCas9-DNMT3A fusion protein can be encoded by the sequence set forth in SEQ ID NO:20. In an aspect, a disclosed dCas9-DNMT3A fusion protein can have a sequence having at least 75%, at least 80%, at least 85%, at least 90%, or at least 95% identity to the sequence set forth in SEQ ID NO:19 or SEQ ID NO:20 or a fragment thereof. In an aspect, a disclosed Cas endonuclease can be dVRER and the polypeptide can be DNMT3A. In an aspect, a disclosed dVRER-DNMT3A fusion protein can have the amino acid sequence set forth SEQ ID NO:38. In an aspect, a disclosed dVRER-DNMT3A fusion protein can be encoded by the sequence set forth in SEQ ID NO:37. In an aspect, a disclosed dVRER-DNMT3A fusion protein can have a sequence having at least 75%, at least 80%, at least 85%, at least 90%, or at least 95% identity to the sequence set forth in SEQ ID NO:38 or SEQ ID NO:37 or a fragment thereof.

In an aspect, a disclosed fusion protein can comprise dCas9 and Kroppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed fusion protein can comprise dVRER and Krüppel-associated box (KRAB), the transcription repression domain (TRD) of Methyl-CpG Binding Protein 2 (MeCP2), or a fusion of KRAB-MeCP2 (KRAB-MeCP2).

In an aspect, a disclosed lentiviral vector can comprise one or more regulatory elements. Regulatory elements are known in the art and can comprise one or more of the following: a Sp1 responsive element, a p2A signal, a woodchuck hepatitis virus post-transcriptional regulatory element, a Phi signal-packaging signal, a rev responsive element, a 5′-LTR, and a 3′-LTR. In an aspect, a disclosed lentiviral vector can comprise two Sp1 response elements, a p2A signal, a woodchuck hepatitis virus post-transcriptional regulatory element, a Phi signal-packaging signal, a rev responsive element, a 5′-LTR, and a 3′-LTR.

Disclosed herein is a lentiviral vector comprising an isolated nucleic acid molecule comprising a nucleic acid sequence encoding (i) a fusion protein and (ii) at least one guide RNA.

In an aspect, a disclosed lentiviral vector can comprise one or more promoters operably linked to the isolated nucleic acid molecule. In an aspect, a disclosed promoter can drive the expression of a gRNA, the Cas9 endonuclease, a polypeptide, or a combination thereof. In an aspect, a disclosed promoter can be a hU6 promoter and a disclosed hU6 promoter can drive expression of a gRNA. In an aspect, a promoter can be an EFS-NC promoter and a disclosed EFS-NC promoter can drive expression of the Cas endonuclease. In an aspect, a disclosed promoter can comprise a hU6 promoter, an EFS-NC promoter, or a combination thereof.

In an aspect, a disclosed Cas endonuclease can comprise Cas9, SpCas9, SaCas9, a variant Cas9, a dCas, or a dCas9. In an aspect, a disclosed Cas9 can comprise the sequence set forth in SEQ ID NO:64 or SEQ ID NO:65. In an aspect, a disclosed Cas9 can have a sequence having at least 75%, at least 80%, at least 85%, at least 90%, or at least 95% identity to the sequence set forth in SEQ ID NO:64 or SEQ ID NO:65 or a fragment thereof.

In an aspect, a disclosed variant Cas9 can comprise VQR, EQR, or VRER. In an aspect a disclosed VRER can comprise the sequence set forth in SEQ ID NO:15. In an aspect, a disclosed VRER can have a sequence having at least 75%, at least 80%, at least 85%, at least 90%, or at least 95% identity to the sequence set forth in SEQ ID NO:15 or a fragment thereof. In an aspect, a disclosed dCas can comprise dVQR, dEQR, or dVRER. In an aspect, a disclosed dCas can comprise the sequence set forth in SEQ ID NO:16. In an aspect, a disclosed dCas9 can have a sequence having at least 75%, at least 80%, at least 85%, at least 90%, or at least 95% identity to the sequence set forth in SEQ ID NO:16 or a fragment thereof. A SpCas9 (3′NGG-PAM sequence) can comprise SpCas9 VQR (3′NGAN or 3′NGNG), SpCas9 EQR (3′NGAG), or SpCas9 VRER (3′NGCG).

In an aspect, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof. In an aspect, a disclosed encoded polypeptide can be histone deacetylase or heterochromatin protein 1. In an aspect, a disclosed encoded polypeptide can comprise transcription repression activity. In an aspect, a disclosed DNMT3A can have the amino acid sequence set forth in SEQ ID NO:17 or the nucleotide sequence set forth in SEQ ID NO:18. In an aspect, a disclosed DNMT3A can have a sequence having at least 75%, at least 80%, at least 85%, at least 90%, or at least 95% identity to the sequence set forth in SEQ ID NO:17 or SEQ ID NO:18 or a fragment thereof.

In an aspect, at least one encoded polypeptide can comprise Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed TRD of MeCP2 can comprise the nucleotide sequence set forth in SEQ ID NO:57 or the amino acid sequence set forth in SEQ ID NO:58. In an aspect, a disclosed TRD of MeCP2 can have a sequence having at least 75%, at least 80%, at least 85%, at least 90%, or at least 95% identity to the sequence set forth in SEQ ID NO:57 or SEQ ID NO:58 or a fragment thereof. In an aspect, a disclosed KRAB-MeCP2 can comprise the nucleotide sequence set forth in SEQ ID NO:62 or the amino acid sequence set forth in SEQ ID NO:63. In an aspect, a disclosed KRAB-MeCP2 can have a sequence having at least 75%, at least 80%, at least 85%, at least 90%, or at least 95% identity to the sequence set forth in SEQ ID NO:62 or SEQ ID NO:63 or a fragment thereof.

In an aspect, a disclosed gRNA can be designed to target exon 4 of the APOE gene or designed to target a protospacer-adjacent motif (PAM) created by a SNP rs429358 in exon 4 of the APOE gene. In an aspect, a disclosed gRNA can have the sequence set forth in any one of SEQ ID NO:05-SEQ ID NO:14, SEQ ID NO:25-SEQ ID NO:28, SEQ ID NO:39-SEQ ID NO:42, and SEQ ID NO:51-SEQ ID NO:52.

In an aspect, a disclosed fusion protein can encode a disclosed Cas endonuclease and a disclosed polypeptide. In an aspect, a disclosed fusion protein can comprise dCas9 and DNMT3A. In an aspect, a dCas9-DNMT3A fusion protein can have the amino acid sequence set forth SEQ ID NO:19. In an aspect, a dCas9-DNMT3A fusion protein can be encoded by the sequence set forth in SEQ ID NO:20. In an aspect, a disclosed dCas9-DNMT3A fusion protein can have a sequence having at least 75%, at least 80%, at least 85%, at least 90%, or at least 95% identity to the sequence set forth in SEQ ID NO:19 or SEQ ID NO:20 or a fragment thereof. In an aspect, a disclosed Cas endonuclease can be dVRER and the polypeptide can be DNMT3A. In an aspect, a disclosed dVRER-DNMT3A fusion protein can have the amino acid sequence set forth SEQ ID NO:38. In an aspect, a disclosed dVRER-DNMT3A fusion protein can be encoded by the sequence set forth in SEQ ID NO:37. In an aspect, a disclosed dVRER-DNMT3A fusion protein can have a sequence having at least 75%, at least 80%, at least 85%, at least 90%, or at least 95% identity to the sequence set forth in SEQ ID NO:38 or SEQ ID NO:37 or a fragment thereof.

In an aspect, a disclosed fusion protein can comprise dCas9 and Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed fusion protein can comprise dVRER and Krüppel-associated box (KRAB), the transcription repression domain (TRD) of Methyl-CpG Binding Protein 2 (MeCP2), or a fusion of KRAB-MeCP2 (KRAB-MeCP2).

In an aspect, a disclosed lentiviral vector can comprise one or more regulatory elements. Regulatory elements are known in the art and can comprise one or more of the following: a Sp1 responsive element, a p2A signal, a woodchuck hepatitis virus post-transcriptional regulatory element, a Phi signal-packaging signal, a rev responsive element, a 5′-LTR, and a 3′-LTR. In an aspect, a disclosed lentiviral vector can comprise two Sp1 response elements, a p2A signal, a woodchuck hepatitis virus post-transcriptional regulatory element, a Phi signal-packaging signal, a rev responsive element, a 5′-LTR, and a 3′-LTR.

### 5. Pharmaceutical Formulations

Disclosed herein is pharmaceutical formulation comprising a disclosed isolated nucleic acid molecule and a pharmaceutically acceptable carrier. Disclosed herein is pharmaceutical formulation comprising an isolated nucleic acid molecule comprising a nucleic acid sequence encoding (i) a Cas endonuclease, (ii) at least one polypeptide having an enzymatic activity, and (iii) at least one guide RNA, and a pharmaceutically acceptable carrier. Disclosed herein is pharmaceutical formulation comprising an isolated nucleic acid molecule comprising a nucleic acid sequence encoding (i) a fusion protein and (ii) at least one guide RNA, and a pharmaceutically acceptable carrier. Disclosed herein is pharmaceutical formulation comprising a disclosed vector and a pharmaceutically acceptable carrier. Disclosed herein is pharmaceutical formulation comprising a disclosed lentiviral vector and a pharmaceutically acceptable carrier.

In an aspect, a disclosed formulation can comprise (i) one or more active agents, (ii) biologically active agents, (iii) one or more pharmaceutically active agents, (iv) one or more immune-based therapeutic agents, (v) one or more clinically approved agents, or (vi) a combination thereof. In an aspect, a disclosed composition can comprise one or more proteasome inhibitors. In an aspect, a disclosed composition can comprise one or more immunosuppressives or immunosuppressive agents. In an aspect, an immunosuppressive agent can be anti-thymocyte globulin (ATG), cyclosporine (CSP), mycophenolate mofetil (MMF), or a combination thereof. In an aspect, a disclosed formulation can comprise a RNA therapeutic. A RNA therapeutic can comprise RNA-mediated interference (RNAi) and/or antisense oligonucleotides (ASO). In an aspect, a disclosed formulation can comprise a disclosed small molecule.

### 6. Host Cells

Disclosed herein is a host cell comprising an isolated nucleic acid molecule comprising a nucleic acid sequence encoding (i) a Cas endonuclease, (ii) at least one polypeptide having an enzymatic activity, and (iii) at least one guide RNA.

Disclosed herein is a host cell comprising an isolated nucleic acid molecule comprising a nucleic acid sequence encoding (i) a fusion protein and (ii) at least one guide RNA.

Disclosed herein is a host cell comprising a lentiviral vector comprising an isolated nucleic acid molecule comprising a nucleic acid sequence encoding (i) a Cas endonuclease, (ii) at least one polypeptide having an enzymatic activity, and (iii) at least one guide RNA.

Disclosed herein is a host cell comprising a lentiviral vector comprising an isolated nucleic acid molecule comprising a nucleic acid sequence encoding (i) a fusion protein and (ii) at least one guide RNA.

Disclosed herein is a host cell comprising plasmid comprising the sequence set forth in any one of SEQ ID NO:21-24, SEQ ID NO:29-36, SEQ ID NO:43-50, SEQ ID NO:53-56, SEQ ID NO:59-61.

In an aspect, a disclosed viral vector or a disclosed lentiviral vector in a disclosed host cell can comprise one or more promoters operably linked to the isolated nucleic acid molecule. In an aspect, a disclosed promoter can drive the expression of a gRNA, the Cas9 endonuclease, a polypeptide, or a combination thereof. In an aspect, a disclosed promoter can be a hU6 promoter and a disclosed hU6 promoter can drive expression of a gRNA. In an aspect, a promoter can be an EFS-NC promoter and a disclosed EFS-NC promoter can drive expression of the Cas endonuclease. In an aspect, a disclosed promoter can comprise a hU6 promoter, an EFS-NC promoter, or a combination thereof.

In an aspect, a disclosed Cas endonuclease can comprise Cas9, SpCas9, SaCas9, a variant Cas9, a dCas, or a dCas9. In an aspect, a disclosed Cas9 can comprise the sequence set forth in SEQ ID NO:64 or SEQ ID NO:65. In an aspect, a disclosed Cas9 can have a sequence having at least 75%, at least 80%, at least 85%, at least 90%, or at least 95% identity to the sequence set forth in SEQ ID NO:64 or SEQ ID NO:65 or a fragment thereof.

In an aspect, a disclosed variant Cas9 can comprise VQR, EQR, or VRER. In an aspect a disclosed VRER can comprise the sequence set forth in SEQ ID NO:15. In an aspect, a disclosed VRER can have a sequence having at least 75%, at least 80%, at least 85%, at least 90%, or at least 95% identity to the sequence set forth in SEQ ID NO:15 or a fragment thereof. In an aspect, a disclosed dCas can comprise dVQR, dEQR, or dVRER. In an aspect, a disclosed dCas can comprise the sequence set forth in SEQ ID NO:16. In an aspect, a disclosed dCas9 can have a sequence having at least 75%, at least 80%, at least 85%, at least 90%, or at least 95% identity to the sequence set forth in SEQ ID NO:16 or a fragment thereof. A SpCas9 (3′NGG-PAM sequence) can comprise SpCas9 VQR (3′NGAN or 3′NGNG), SpCas9 EQR (3′NGAG), or SpCas9 VRER (3′NGCG).

In an aspect, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof. In an aspect, a disclosed encoded polypeptide can be histone deacetylase or heterochromatin protein 1. In an aspect, a disclosed encoded polypeptide can comprise transcription repression activity. In an aspect, a disclosed DNMT3A can have the amino acid sequence set forth in SEQ ID NO:17 or the nucleotide sequence set forth in SEQ ID NO:18. In an aspect, a disclosed DNMT3A can have a sequence having at least 75%, at least 80%, at least 85%, at least 90%, or at least 95% identity to the sequence set forth in SEQ ID NO:17 or SEQ ID NO:18 or a fragment thereof.

In an aspect, at least one encoded polypeptide can comprise Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed TRD of MeCP2 can comprise the nucleotide sequence set forth in SEQ ID NO:57 or the amino acid sequence set forth in SEQ ID NO:58. In an aspect, a disclosed TRD of MeCP2 can have a sequence having at least 75%, at least 80%, at least 85%, at least 90%, or at least 95% identity to the sequence set forth in SEQ ID NO:57 or SEQ ID NO:58 or a fragment thereof. In an aspect, a disclosed KRAB-MeCP2 can comprise the nucleotide sequence set forth in SEQ ID NO:62 or the amino acid sequence set forth in SEQ ID NO:63. In an aspect, a disclosed KRAB-MeCP2 can have a sequence having at least 75%, at least 80%, at least 85%, at least 90%, or at least 95% identity to the sequence set forth in SEQ ID NO:62 or SEQ ID NO:63 or a fragment thereof.

In an aspect, a disclosed gRNA can be designed to target exon 4 of the APOE gene or designed to target a protospacer-adjacent motif (PAM) created by a SNP rs429358 in exon 4 of the APOE gene. In an aspect, a disclosed gRNA can have the sequence set forth in any one of SEQ ID NO:05-SEQ ID NO:14, SEQ ID NO:25-SEQ ID NO:28, SEQ ID NO:39-SEQ ID NO:42, and SEQ ID NO:51-SEQ ID NO:52.

In an aspect, a disclosed fusion protein can encode a disclosed Cas endonuclease and a disclosed polypeptide. In an aspect, a disclosed fusion protein can comprise dCas9 and DNMT3A. In an aspect, a dCas9-DNMT3A fusion protein can have the amino acid sequence set forth SEQ ID NO:19. In an aspect, a dCas9-DNMT3A fusion protein can be encoded by the sequence set forth in SEQ ID NO:20. In an aspect, a disclosed dCas9-DNMT3A fusion protein can have a sequence having at least 75%, at least 80%, at least 85%, at least 90%, or at least 95% identity to the sequence set forth in SEQ ID NO:19 or SEQ ID NO:20 or a fragment thereof. In an aspect, a disclosed Cas endonuclease can be dVRER and the polypeptide can be DNMT3A. In an aspect, a disclosed dVRER-DNMT3A fusion protein can have the amino acid sequence set forth SEQ ID NO:38. In an aspect, a disclosed dVRER-DNMT3A fusion protein can be encoded by the sequence set forth in SEQ ID NO:37. In an aspect, a disclosed dVRER-DNMT3A fusion protein can have a sequence having at least 75%, at least 80%, at least 85%, at least 90%, or at least 95% identity to the sequence set forth in SEQ ID NO:38 or SEQ ID NO:37 or a fragment thereof.

In an aspect, a disclosed fusion protein can comprise dCas9 and Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed fusion protein can comprise dVRER and Krüppel-associated box (KRAB), the transcription repression domain (TRD) of Methyl-CpG Binding Protein 2 (MeCP2), or a fusion of KRAB-MeCP2 (KRAB-MeCP2).

In an aspect, a disclosed viral vector or a disclosed lentiviral vector in a disclosed host cell can comprise one or more regulatory elements. Regulatory elements are known in the art and can comprise one or more of the following: a Sp1 responsive element, a p2A signal, a woodchuck hepatitis virus post-transcriptional regulatory element, a Phi signal-packaging signal, a rev responsive element, a 5′-LTR, and a 3′-LTR. In an aspect, a disclosed viral vector or a disclosed lentiviral vector can comprise two Sp1 response elements, a p2A signal, a woodchuck hepatitis virus post-transcriptional regulatory element, a Phi signal-packaging signal, a rev responsive element, a 5′-LTR, and a 3′-LTR.

7. Guide RNAs (gRNAs)

Disclosed herein is a guide RNA comprising the sequence set forth in any one of SEQ ID NO:05-SEQ ID NO: 14. Disclosed herein is a guide RNA comprising the sequence set forth in any one of SEQ ID NO:25-SEQ ID NO:28. Disclosed herein is a guide RNA comprising the sequence set forth in any one of SEQ ID NO:39-SEQ ID NO:42. Disclosed herein is a guide RNA comprising the sequence set forth in any one of SEQ ID NO:51-SEQ ID NO:52. Disclosed gRNAs are listed below.

| SEQ | | |
|---|---|---|
| ID NO. | gRNA Sequence | Description |
| 5 | gacagggggagccctataat | gRNA1 targeting promoter region of ApoE using |
| | | SpCas9 |
| 6 | tcaggagagctactcggggt | gRNA2 targeting promoter region of ApoE using |
| | | SpCas9 |
| 7 | actgggatgtaagccatagc | gRNA3 targeting promoter region of ApoE using |
| | | SpCas9 |
| 8 | gttggagcttagaatgtgaa | gRNA4 targeting promoter region of ApoE using |
| | | SpCas9 |
| 9 | gccctatccctgggggaggg | gRNA1 targeting promoter region of ApoE using VRER |
| | | Cas9 |
| 10 | tcgggcttggggagaggagg | gRNA2 targeting promoter region of ApoE using VRER |
| | | Cas9 |
| 11 | ctctccccaccccaccttct | gRNA3 targeting promoter region of ApoE using VRER |
| | | Cas9 |
| 12 | tgtgaagggagaatgaggaa | gRNA4 targeting promoter region of ApoE using VRER |
| | | Cas9 |
| 13 | ggcgaggagctgttcaccg | gRNA targeting GFP ORF using SpCas9 |
| 14 | gccacaagttcagcgtgtcc | gRNA targeting GFP ORF using VRER-SpCas9 |
| 25 | gacagggggagccctataat | gRNA1 cloned into pBK1026 and pBK1030 plasmids |
| 26 | tcaggagagctactcggggt | gRNA2 cloned into pBK1027 and pBK1031 plasmids |
| 27 | actgggatgtaagccatagc | gRNA3 cloned into pBK1028 and pBK1032 plasmids |
| 28 | gttggagcttagaatgtgaa | gRNA4 cloned into pBK1029 and pBK1033 plasmids |
| 39 | gccctatccctgggggaggg | gRNA1 cloned into pBK1105 and pBK1109 plasmids |
| 40 | tcgggcttggggagaggagg | gRNA2 cloned into pBK1106 and pBK1110 plasmids |
| 41 | ctctccccaccccaccttct | gRNA3 cloned into pBK1107 and pBK1111 plasmids |
| 42 | tgtgaagggagaatgaggaa | gRNA4 cloned into pBK1108 and pBK1112 plasmids |
| 51 | gggcgcggacatggaggacg | gRNA1 cloned into pBK1426 and pBK1428 plasmids |
| 52 | gggcgcggacatggaggacg | gRNA2 cloned into pBK1427 and pBK1429 plasmids |

As known to the art, a gRNA provides the targeting of a CRISPR/Cas9-based epigenome modifying system. A guide RNA is a specific RNA sequence that recognizes the target DNA region of interest (such as, for example, APOE e4 allele) and directs the Cas endonuclease there for editing. The gRNA is made up of two parts: crispr RNA (crRNA), a 17-20 nucleotide sequence complementary to the target DNA, and a tracer RNA, which serves as a binding scaffold for the Cas nuclease. The CRISPR-associated (Cas) protein is a non-specific endonuclease, which can be directed to the specific DNA locus by a gRNA (where it makes a double-strand break).

In an aspect, a disclosed gRNA can serve to direct a disclosed endonucleases or a disclosed fusion product having an endonuclease to a target area of interest (such as, for example, the promoter of the APOE gene or the APOE e4 allele).

### 8. Plasmids

Disclosed herein is a plasmid comprising the sequence set forth in any of SEQ ID NO:21-SEQ ID NO:24. Disclosed herein is a plasmid comprising the sequence set forth in any of SEQ ID NO:29-SEQ ID NO:36. Disclosed herein is a plasmid comprising the sequence set forth in any of SEQ ID NO:43-SEQ ID NO:50. Disclosed herein is a plasmid comprising the sequence set forth in any of SEQ ID NO:53-SEQ ID NO:56. Disclosed herein is a plasmid comprising the sequence set forth in any of SEQ ID NO:59-SEQ ID NO:61. Plasmids disclosed herein include but are not limited to those listed below.

| SEQ ID NO | Plasmid Description |
|---|---|
| 21 | pBK546 carrying dCas9-DNMT3A fused transgene linked to puromycin |
| | reporter via p2A cleavage signal |
| 22 | pBK539 carrying dCas9-DNMT3A fused transgene linked to GFP reporter via |
| | p2A cleavage signal |
| 23 | pBK500 carrying all-in-one lentiviral vector containing fusion protein and |
| | gRNA4 |
| 24 | pBK744 carrying dCas9-DNMT3A fused transgene linked to GFP reporter via |
| | p2A cleavage signal and carrying gRNA3 targeting rat/mouse intron Snca- |
| | intron 1 sequences |
| 29 | pBK1026 carrying dCas9-(active) DNMT3A vector targeting promoter region |
| | of ApoE gene and carrying gRNA1 and puromycin reporter separated via p2a |
| | signal from DNMT3A and SpCas9 is present |
| 30 | pBK1027 carrying dCas9-(active) DNMT3A vector targeting promoter region |
| | of ApoE gene and carrying gRNA2 and puromycin reporter separated via p2a |
| | signal from DNMT3A and SpCas9 is present |
| 31 | pBK1028 carrying dCas9-(active) DNMT3A vector targeting promoter region |
| | of ApoE gene and carrying gRNA3 and puromycin reporter separated via p2a |
| | signal from DNMT3A and SpCas9 is present |
| 32 | pBK1029 carrying dCas9-(active) DNMT3A vector targeting promoter region |
| | of ApoE gene and carrying gRNA4 and puromycin reporter separated via p2a |
| | signal from DNMT3A and SpCas9 is present |
| 33 | pBK1030 carrying dCas9-(inactive) DNMT3A vector targeting promoter |
| | region of ApoE gene and carrying gRNA1 and puromycin reporter separated |
| | via p2a signal from DNMT3A and SpCas9 is present |
| 34 | pBK1031 carrying dCas9-(inactive) DNMT3A vector targeting promoter |
| | region of ApoE gene and carrying gRNA2 and puromycin reporter separated |
| | via p2a signal from DNMT3A and SpCas9 is present |
| 35 | pBK1032 carrying dCas9-(inactive) DNMT3A vector targeting promoter |
| | region of ApoE gene and carrying gRNA3 and puromycin reporter separated |
| | via p2a signal from DNMT3A and SpCas9 is present |
| 36 | pBK1033 carrying dCas9-(inactive) DNMT3A vector targeting promoter |
| | region of ApoE gene and carrying gRNA4 and puromycin reporter separated |
| | via p2a signal from DNMT3A and SpCas9 is present |
| 43 | pBK1105 carrying dCas9-VRER (active) DNMT3A vector targeting promoter |
| | region of ApoE gene and carrying gRNA1 and puromycin reporter separated |
| | via p2a signal from DNMT3A and SpCas9 is present |
| 44 | pBK1106 carrying dCas9-VRER (active) DNMT3A vector targeting promoter |
| | region of ApoE gene and carrying gRNA2 and puromycin reporter separated |
| | via p2a signal from DNMT3A and SpCas9 is present |
| 45 | pBK1107 carrying dCas9-VRER (active) DNMT3A vector targeting promoter |
| | region of ApoE gene and carrying gRNA3 and puromycin reporter separated |
| | via p2a signal from DNMT3A and SpCas9 is present |
| 46 | pBK1108 carrying dCas9-VRER (active) DNMT3A vector targeting promoter |
| | region of ApoE gene and carrying gRNA4 and puromycin reporter separated |
| | via p2a signal from DNMT3A and SpCas9 is present |
| 47 | pBK1109 carrying dCas9-VRER (inactive) DNMT3A vector targeting |
| | promoter region of ApoE gene and carrying gRNA1 and puromycin reporter |
| | separated via p2a signal from DNMT3A and SpCas9 is present |
| 48 | pBK1110 carrying dCas9-VRER (inactive) DNMT3A vector targeting |
| | promoter region of ApoE gene and carrying gRNA2 and puromycin reporter |
| | separated via p2a signal from DNMT3A and SpCas9 is present |
| 49 | pBK1111 carrying dCas9-VRER (inactive) DNMT3A vector targeting |
| | promoter region of ApoE gene and carrying gRNA3 and puromycin reporter |
| | separated via p2a signal from DNMT3A and SpCas9 is present |
| 50 | pBK1112 carrying dCas9-VRER (inactive) DNMT3A vector targeting |
| | promoter region of ApoE gene and carrying gRNA3 and puromycin reporter |
| | separated via p2a signal from DNMT3A and SpCas9 is present |
| 53 | pBK1426 carrying dCas9-VRER (active) DNMT3A vector targeting SNP |
| | region of ApoE4 and carrying gRNA1 and puromycin reporter separated via |
| | p2a signal from DNMT3A and VRER-SpCas9 is present |
| 54 | pBK1427 carrying dCas9-VRER (active) DNMT3A vector targeting SNP |
| | region of ApoE4 and carrying gRNA2 and puromycin reporter separated via |
| | p2a signal from DNMT3A and VRER-SpCas9 is present |
| 55 | pBK1428 carrying dCas9-VRER (inactive) DNMT3A vector targeting SNP |
| | region of ApoE4 and carrying gRNA1 and puromycin reporter separated via |
| | p2a signal from DNMT3A and VRER-SpCas9 is present |
| 56 | pBK1429 carrying dCas9-VRER DNMT3A vector targeting SNP region of |
| | ApoE4 and carrying gRNA2 and puromycin reporter separated via p2a signal |
| | from DNMT3A and VRER-SpCas9 is present |
| 59 | pBK1531 carrying a Lentiviral vector with dCas9-VRER-MeCP2 |
| | Transcription Repression Domain (TRD) and gRNA1 |
| 60 | pBK1532 carrying a Lentiviral vector with dCas9-VRER-MeCP2 |
| | Transcription Repression Domain (TRD) and gRNA2 |
| 61 | pBK1536 carrying a Lentiviral vector with dCas9-VRER-MeCP2 |
| | Transcription Repression Domain (TRD) without gRNA |

In an aspect, a disclosed pBK546 plasmid can comprise the sequence set forth below or a sequence having 70%, 75%, 80%, 85%, 90%, 95%, or more identity to the sequence set forth

(SEQ ID NO: 21)
gtcgacggatcgggagatctcccgatcccctatggtgcactctcagtacaatctgctctgatgccgcatagttaagccagtatctgctccctg
cttgtgtgttggaggtcgctgagtagtgcgcgagcaaaatttaagctacaacaaggcaaggcttgaccgacaattgcatgaagaatctgctt
agggttaggcgttttgcgctgcttcgcgatgtacgggccagatatacgcgttgacattgattattgactagttattaatagtaatcaattacggg
gtcattagttcatagcccatatatggagttccgcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtacatcaatgggcgtggata
gcggtttgactcacggggatttccaagtctccaccccattgacgtcaatgggagtttgttttggcaccaaaatcaacgggactttccaaaatgt
cgtaacaactccgccccattgacgcaaatgggcggtaggcgtgtacggtgggaggtctatataagcagcgcgttttgcctgtactgggtct
ctctggttagaccagatctgagcctgggagctctctggctaactagggaacccactgcttaagcctcaataaagcttgccttgagtgcttcaa
gtagtgtgtgcccgtctgttgtgtgactctggtaactagagatccctcagacccttttagtcagtgtggaaaatctctagcagtggcgcccgaa
cagggacttgaaagcgaaagggaaaccagaggagctctctcgacgcaggactcggcttgctgaagcgcgcacggcaagaggcgagg
ggcggcgactggtgagtacgccaaaaattttgactagcggaggctagaaggagagagatgggtgcgagagcgtcagtattaagcgggg
gagaattagatcgcgatgggaaaaaattcggttaaggccagggggaaagaaaaaatataaattaaaacatatagtatgggcaagcaggga
gctagaacgattcgcagttaatcctggcctgttagaaacatcagaaggctgtagacaaatactgggacagctacaaccatcccttcagacag
gatcagaagaacttagatcattatataatacagtagcaaccctctattgtgtgcatcaaaggatagagataaaagacaccaaggaagctttag
acaagatagaggaagagcaaaacaaaagtaagaccaccgcacagcaagcggccgctgatcttcagacctggaggaggagatatgagg
gacaattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatctgttgca
actcacagtctggggcatcaagcagctccaggcaagaatcctggctgtggaaagatacctaaaggatcaacagctcctggggatttgggg
ttgctctggaaaactcatttgcaccactgctgtgccttggaatgctagttggagtaataaatctctggaacagatttggaatcacacgacctgg
atggagtgggacagagaaattaacaattacacaagcttaatacactccttaattgaagaatcgcaaaaccagcaagaaaagaatgaacaag
aattattggaattagataaatgggcaagtttgtggaattggtttaacataacaaattggctgtggtatataaaattattcataatgatagtaggag
gcttggtaggtttaagaatagtttttgctgtactttctatagtgaatagagttaggcagggatattcaccattatcgtttcagacccacctcccaac
cccgaggggacccgacaggcccgaaggaatagaagaagaaggtggagagagagacagagacagatccattcgattagtgaacggatc
ggcactgcgtgcgccaattctgcagacaaatggcagtattcatccacaattttaaaagaaaaggggggattggggggtacagtgcagggg
aaagaatagtagacataatagcaacagacatacaaactaaagaattacaaaaacaaattacaaaaattcaaaattttcgggtttattacaggg
acagcagagatccagtttggttaattaatggggggacgttaacggggcggaacggtaccgagggcctatttcccatgattccttcatatttg
catatacgatacaaggctgttagagagataattagaattaatttgactgtaaacacaaagatattagtacaaaatacgtgacgtagaaagtaataatt
tcttgggtagtttgcagttttaaaattatgttttaaaatggactatcatatgcttaccgtaacttgaaagtatttcgatttcttggctttatatatct
tgtggaaaggacgaaacaccggagacgtgtacacgtctctgttttagagctagaaatagcaagttaaaataaggctagtccgttatcaacttg
aaaaagtggcaccgagtcggtgcttttttgaattcgctagctaggtcttgaaaggagtgggaattggctccggtgcccgtcagtgggcaga
gcgcacatcgcccacagtccccgagaagttggggggaggggtcggcaattgatccggtgcctagagaaggtggcgcggggtaaactg
ggaaagtgatgtcgtgtactggctccgcctttttcccgagggtgggggagaaccgtatataagtgcagtagtcgccgtgaacgttctttttcg
caacgggtttgccgccagaacacaggaccggtgccaccatggactataaggaccacgacggagactacaaggatcatgatattgattaca
aagacgatgacgataagatggccccaaagaagaagcggaaggtcggtatccacggagtcccagcagccgacaagaagtacagcatcg
gcctggccatcggcaccaactctgtgggctgggccgtgatcaccgacgagtacaaggtgcccagcaagaaattcaaggtgctgggcaac
accgaccggcacagcatcaagaagaacctgatcggagccctgctgttcgacagcggcgaaacagccgaggccacccggctgaagaga
accgccagaagaagatacaccagacggaagaaccggatctgctatctgcaagagatcttcagcaacgagatggccaaggtggacgaca
gcttcttccacagactggaagagtccttcctggtggaagaggataagaagcacgagcggcaccccatcttcggcaacatcgtggacgagg
tggcctaccacgagaagtaccccaccatctaccacctgagaaagaaactggtggacagcaccgacaaggccgacctgcggctgatctat
ctggccctggcccacatgatcaagttccggggccacttcctgatcgagggcgacctgaaccccgacaacagcgacgtggacaagctgtt
catccagctggtgcagacctacaaccagctgttcgaggaaaaccccatcaacgccagcggcgtggacgccaaggccatcctgtctgcca
gactgagcaagagcagacggctggaaaatctgatcgcccagctgcccggcgagaagaagaatggcctgttcggcaacctgattgccctg
agcctgggcctgacccccaacttcaagagcaacttcgacctggccgaggatgccaaactgcagctgagcaaggacacctacgacgacg
acctggacaacctgctggcccagatcggcgaccagtacgccgacctgtttctggccgccaagaacctgtccgacgccatcctgctgagcg
acatcctgagagtgaacaccgagatcaccaaggcccccctgagcgcctctatgatcaagagatacgacgagcaccaccaggacctgacc
ctgctgaaagctctcgtgcggcagcagctgcctgagaagtacaaagagattttcttcgaccagagcaagaacggctacgccggctacattg
acggcggagccagccaggaagagttctacaagttcatcaagcccatcctggaaaagatggacggcaccgaggaactgctcgtgaagct
gaacagagaggacctgctgcggaagcagcggaccttcgacaacggcagcatcccccaccagatccacctgggagagctgcacgccatt
ctgcggcggcaggaagatttttacccattcctgaaggacaaccgggaaaagatcgagaagatcctgaccttccgcatcccctactacgtgg
gccctctggccaggggaaacagcagattcgcctggatgaccagaaagagcgaggaaaccatcaccccctggaacttcgaggaagtggt
ggacaagggcgcttccgcccagagcttcatcgagcggatgaccaacttcgataagaacctgcccaacgagaaggtgctgcccaagcac
agcctgctgtacgagtacttcaccgtgtataacgagctgaccaaagtgaaatacgtgaccgagggaatgagaaagcccgccttcctgagc
ggcgagcagaaaaaggccatcgtggacctgctgttcaagaccaaccggaaagtgaccgtgaagcagctgaaagaggactacttcaaga
aaatcgagtgcttcgactccgtggaaatctccggcgtggaagatcggttcaacgcctccctgggcacataccacgatctgctgaaaattatc
aaggacaaggacttcctggacaatgaggaaaacgaggacattctggaagatatcgtgctgaccctgacactgtttgaggacagagagatg
atcgaggaacggctgaaaacctatgcccacctgttcgacgacaaagtgatgaagcagctgaagcggcggagatacaccggctggggca
ggctgagccggaagctgatcaacggcatccgggacaagcagtccggcaagacaatcctggatttcctgaagtccgacggcttcgccaac
agaaacttcatgcagctgatccacgacgacagcctgacctttaaagaggacatccagaaagcccaggtgtccggccagggcgatagcct
gcacgagcacattgccaatctggccggcagccccgccattaagaagggcatcctgcagacagtgaaggtggtggacgagctcgtgaaa
gtgatgggccggcacaagcccgagaacatcgtgatcgaaatggccagagagaaccagaccacccagaagggacagaagaacagccg
cgagagaatgaagcggatcgaagagggcatcaaagagctgggcagccagatcctgaaagaacaccccgtggaaaacacccagctgca
gaacgagaagctgtacctgtactacctgcagaatggggggatatgtacgtggaccaggaactggacatcaaccggctgtccgactacga
tgtggacgctatcgtgcctcagagctttctgaaggacgactccatcgacaacaaggtgctgaccagaagcgacaagaaccggggcaaga
gcgacaacgtgccctccgaagaggtcgtgaagaagatgaagaactactggcggcagctgctgaacgccaagctgattacccagagaaa
gttcgacaatctgaccaaggccgagagaggcggcctgagcgaactggataaggccggcttcatcaagagacagctggtggaaacccgg
cagatcacaaagcacgtggcacagatcctggactcccggatgaacactaagtacgacgagaatgacaagctgatccgggaagtgaaagt
gatcaccctgaagtccaagctggtgtccgatttccggaaggatttccagttttacaaagtgcgcgagatcaacaactaccaccacgcccacg
acgcctacctgaacgccgtcgtgggaaccgccctgatcaaaaagtaccctaagctggaaagcgagttcgtgtacggcgactacaaggtgt
acgacgtgcggaagatgatcgccaagagcgagcaggaaatcggcaaggctaccgccaagtacttcttctacagcaacatcatgaacttttt
caagaccgagattaccctggccaacggcgagatccggaagcggcctctgatcgagacaaacggcgaaaccggggagatcgtgtggga
taagggccgggattttgccaccgtgcggaaagtgctgagcatgccccaagtgaatatcgtgaaaaagaccgaggtgcagacaggcggct
tcagcaaagagtctatcctgcccaagaggaacagcgataagctgatcgccagaaagaaggactgggaccctaagaagtacggggcttc
gacagccccaccgtggcctattctgtgctggtggtggccaaagtggaaaagggcaagtccaagaaactgaagagtgtgaaagagctgct
ggggatcaccatcatggaaagaagcagcttcgagaagaatcccatcgactttctggaagccaagggctacaaagaagtgaaaaaggacc
tgatcatcaagctgcctaagtactccctgttcgagctggaaaacggccggaagagaatgctggcctctgccggcgaactgcagaagggaa
acgaactggccctgccctccaaatatgtgaacttcctgtacctggccagccactatgagaagctgaagggctcccccgaggataatgagca
gaaacagctgtttgtggaacagcacaagcactacctggacgagatcatcgagcagatcagcgagttctccaagagagtgatcctggccga
cgctaatctggacaaagtgctgtccgcctacaacaagcaccgggataagcccatcagagagcaggccgagaatatcatccacctgtttac
cctgaccaatctgggagcccctgccgccttcaagtactttgacaccaccatcgaccggaagaggtacaccagcaccaaagaggtgctgg
acgccaccctgatccaccagagcatcaccggcctgtacgagacacggatcgacctgtctcagctgggaggcgacaaaaggccggcgg
ccacgaaaaaggccggacaggccaaaaagaaaaagctcgagggcggaggcgggagcggatccccctcccggctccagatgttcttcg
ctaataaccacgaccaggaatttgaccctccaaaggtttacccacctgtcccagctgagaagaggaagcccatccgggtgctgtctctcttt
gatggaatcgctacagggctcctggtgctgaaggacttgggcattcaggtggaccgctacattgcctcggaggtgtgtgaggactccatca
cggtgggcatggtgcggcaccaggggaagatcatgtacgtcggggacgtccgcagcgtcacacagaagcatatccaggagtggggcc
cattcgatctggtgattgggggcagtccctgcaatgacctctccatcgtcaaccctgctcgcaagggcctctacgagggcactggccggct
cttctttgagttctaccgcctcctgcatgatgcgcggcccaaggagggagatgatcgccccttcttctggctctttgagaatgtggtggccatg
ggcgttagtgacaagagggacatctcgcgatttctcgagtccaaccctgtgatgattgatgccaaagaagtgtcagctgcacacagggccc
gctacttctggggtaaccttcccggtatgaacaggccgttggcatccactgtgaatgataagctggagctgcaggagtgtctggagcatgg
caggatagccaagttcagcaaagtgaggaccattactacgaggtcaaactccataaagcagggcaaagaccagcattttcctgtgttcatg
aatgagaaagaggacatcttatggtgcactgaaatggaaagggtatttggtttcccagtccactatactgacgtgtccaacatgagccgcttg
gcgaggcagagactgctgggccggtcatggagcgtgccagtcatccgccacctcttcgctccgctgaaggagtattttgcgtgtgtgtccg
gccggcccggatccggcgcaacaaacttctctctgctgaaacaagccggagatgtcgaagagaatcctggaccgaccgagtacaagcc
cacggtgcgcctcgccacccgcgacgacgtccccagggccgtacgcaccctcgccgccgegttcgccgactaccccgccacgcgcca
caccgtcgatccggaccgccacatcgagcgggtcaccgagctgcaagaactcttcctcacgcgcgtcgggctcgacatcggcaaggtgt
gggtcgcggacgacggcgccgcggtggcggtctggaccacgccggagagcgtcgaagcgggggcggtgttcgccgagatcggccc
gcgcatggccgagttgagcggttcccggctggccgcgcagcaacagatggaaggcctcctggcgccgcaccggcccaaggagcccg
cgtggttcctggccaccgtcggagtctcgcccgaccaccagggcaagggtctgggcagcgccgtcgtgctccccggagtggaggcgg
ccgagcgcgccggggtgcccgccttcctggagacctccgcgccccgcaacctccccttctacgageggcteggcttcaccgtcaccgcc
gacgtcgaggtgcccgaaggaccgcgcacctggtgcatgacccgcaagcccggtgcctgaacgcgttaagtcgacaatcaacctctggatt
acaaaatttgtgaaagattgactggtattcttaactatgttgctccttttacgctatgtggatacgctgctttaatgcctttgtatcatgctattgc
ttcccgtatggctttcattttctcctccttgtataaatcctggttgctgtctctttatgaggagttgtggcccgttgtcaggcaacgtggcgtggtgt
gcactgtgtttgctgacgcaacccccactggttggggcattgccaccacctgtcagctcctttccgggactttcgctttccccctccctattgcc
acggcggaactcatcgccgcctgccttgcccgctgctggacaggggctcggctgttgggcactgacaattccgtggtgttgtcggggaaa
tcatcgtcctttccttggctgctcgcctgtgttgccacctggattctgcgcgggacgtccttctgctacgtcccttcggccctcaatccagcgg
accttccttcccgcggcctgctgccggctctgcggcctcttccgcgtcttcgccttcgccctcagacgagtcggatctccctttgggccgcct
ccccgcgtcgactttaagaccaatgacttacaaggcagctgtagatcttagccactttttaaaagaaaaggggggactggaagggctaattc
actcccaacgaagacaagatctgctttttgcttgtactgggtctctctggttagaccagatctgagcctgggagctctctggctaactagggaa
cccactgcttaagcctcaataaagcttgccttgagtgcttcaagtagtgtgtgcccgtctgttgtgtgactctggtaactagagatccctcagac
ccttttagtcagtgtggaaaatctctagcagggcccgtttaaacccgctgatcagcctcgactgtgccttctagttgccagccatctgttgtttg
cccctcccccgtgccttccttgaccctggaaggtgccactcccactgtcctttcctaataaaatgaggaaattgcatcgcattgtctgagtagg
tgtcattctattctggggggtggggggggcaggacagcaagggggaggattgggaagacaatagcaggcatgctggggatgcggtgg
gctctatggcttctgaggcggaaagaaccagctggggctctagggggtatccccacgcgccctgtagcggcgcattaagcgcggcgggt
gtggtggttacgcgcagcgtgaccgctacacttgccagcgccctagcgcccgctcctttcgctttettcccttcctttctcgccacgttcgccg
gctttccccgtcaagctctaaatcgggggctccctttagggttccgatttagtgctttacggcacctcgaccccaaaaaacttgattagggtga
tggttcacgtagtgggccatcgccctgatagacggtttttcgccctttgacgttggagtccacgttctttaatagtggactcttgttccaaactgg
aacaacactcaaccctatctcggtctattcttttgatttataagggattttgccgatttcggcctattggttaaaaaatgagctgatttaacaaaaat
ttaacgcgaattaattctgtggaatgtgtgtcagttagggtgtggaaagtccccaggctccccagcaggcagaagtatgcaaagcatgcatc
tcaattagtcagcaaccaggtgtggaaagtccccaggctccccagcaggcagaagtatgcaaagcatgcatctcaattagtcagcaaccat
agtcccgcccctaactccgcccatcccgcccctaactccgcccagttccgcccattctccgccccatggctgactaattttttttatttatgcag
aggccgaggccgcctctgcctctgagctattccagaagtagtgaggaggcttttttggaggcctaggcttttgcaaaaagctcccgggagc
ttgtatatccattttcggatctgatcagcacgtgttgacaattaatcatcggcatagtatatcggcatagtataatacgacaaggtgaggaacta
aaccatggccaagttgaccagtgccgttccggtgctcaccgcgcgcgacgtcgccggagcggtcgagttctggaccgaccggctcgggt
tctcccgggacttcgtggaggacgacttcgccggtgtggtccgggacgacgtgaccctgttcatcagcgcggtccaggaccaggtggtg
ccggacaacaccctggcctgggtgtgggtgcgcggcctggacgagctgtacgccgagtggtcggaggtcgtgtccacgaacttccggg
acgcctccgggccggccatgaccgagatcggcgagcagccgtggggggggagttcgccctgcgcgacccggccggcaactgcgtg
cacttcgtggccgaggagcaggactgacacgtgctacgagatttcgattccaccgccgccttctatgaaaggttgggcttcggaatcgttttc
cgggacgccggctggatgatcctccagcgcggggatctcatgctggagttcttcgcccaccccaacttgtttattgcagcttataatggttacaa
ataaagcaatagcatcacaaatttcacaaataaagcatttttttcactgcattctagttgtggtttgtccaaactcatcaatgtatcttatcatgtc
tgtataccgtcgacctctagctagagcttggcgtaatcatggtcatagctgtttcctgtgtgaaattgttatccgctcacaattccacacaacata
cgagccggaagcataaagtgtaaagcctggggtgcctaatgagtgagctaactcacattaattgcgttgcgctcactgcccgctttccagtc
gggaaacctgtcgtgccagctgcattaatgaatcggccaacgcgcggggagaggcggtttgcgtattgggcgctcttccgcttcctcgctc
actgactcgctgcgctcggtcgttcggctgcggcgagcggtatcagctcactcaaaggcggtaatacggttatccacagaatcaggggata
acgcaggaaagaacatgtgagcaaaaggccagcaaaaggccaggaaccgtaaaaaggccgcgttgctggcgtttttccataggctccgc
ccccctgacgagcatcacaaaaatcgacgctcaagtcagaggtggcgaaacccgacaggactataaagataccaggcgtttccccctgg
aagctccctcgtgcgctctcctgttccgaccctgccgcttaccggatacctgtccgcctttctcccttcgggaagcgtggcgctttctcatagc
tcacgctgtaggtatctcagttcggtgtaggtcgttcgctccaagctgggctgtgtgcacgaaccccccgttcagcccgaccgctgcgcctt
atccggtaactatcgtcttgagtccaacccggtaagacacgacttatcgccactggcagcagccactggtaacaggattagcagagcgag
gtatgtaggcggtgctacagagttcttgaagtggtggcctaactacggctacactagaagaacagtatttggtatctgcgctctgctgaagcc
agttaccttcggaaaaagagttggtagctcttgatccggcaaacaaaccaccgctggtagcggtggtttttttgtttgcaagcagcagattac
gcgcagaaaaaaaggatctcaagaagatcctttgatcttttctacggggtctgacgctcagtggaacgaaaactcacgttaagggattttggt
catgagattatcaaaaaggatcttcacctagatccttttaaattaaaaatgaagttttaaatcaatctaaagtatatatgagtaaacttggtctgac
agttaccaatgcttaatcagtgaggcacctatctcagcgatctgtctatttcgttcatccatagttgcctgactccccgtcgtgtagataactacg
atacgggagggcttaccatctggccccagtgctgcaatgataccgcgagacccacgctcaccggctccagatttatcagcaataaaccag
ccagccggaagggccgagcgcagaagtggtcctgcaactttatccgcctccatccagtctattaattgttgccgggaagctagagtaagta
gttcgccagttaatagtttgcgcaacgttgttgccattgctacaggcatcgtggtgtcacgctcgtcgtttggtatggcttcattcagctccggtt
cccaacgatcaaggcgagttacatgatcccccatgttgtgcaaaaaagcggttagctccttcggtcctccgatcgttgtcagaagtaagttgg
ccgcagtgttatcactcatggttatggcagcactgcataattctcttactgtcatgccatccgtaagatgcttttctgtgactggtgagtactcaa
ccaagtcattctgagaatagtgtatgcggcgaccgagttgctcttgcccggcgtcaatacgggataataccgcgccacatagcagaacttta
aaagtgctcatcattggaaaacgttcttcggggcgaaaactctcaaggatcttaccgctgttgagatccagttcgatgtaacccactcgtgca
cccaactgatcttcagcatcttttactttcaccagcgtttctgggtgagcaaaaacaggaaggcaaaatgccgcaaaaaagggaataaggg
cgacacggaaatgttgaatactcatactcttcctttttcaatattattgaagcatttatcagggttattgtctcatgagcggatacatatttgaat
gtatttagaaaaataaacaaataggggttccgcgcacatttccccgaaaagtgccacctgac.

In an aspect, a disclosed pBK539 plasmid can comprise the sequence set forth below or a sequence having 70%, 75%, 80%, 85%, 90%, 95%, or more identity to the sequence set forth

(SEQ ID NO: 22)
gtcgacggatcgggagatctcccgatcccctatggtgcactctcagtacaatctgctctgatgccgcatagttaagccagtatctgctccctg
cttgtgtgttggaggtcgctgagtagtgcgcgagcaaaatttaagctacaacaaggcaaggcttgaccgacaattgcatgaagaatctgctt
agggttaggcgttttgcgctgcttcgcgatgtacgggccagatatacgcgttgacattgattattgactagttattaatagtaatcaattacggg
gtcattagttcatagcccatatatggagttccgcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtacatcaatgggcgtggata
gcggtttgactcacggggatttccaagtctccaccccattgacgtcaatgggagtttgttttggcaccaaaatcaacgggactttccaaaatgt
cgtaacaactccgccccattgacgcaaatgggcggtaggcgtgtacggtgggaggtctatataagcagcgcgttttgcctgtactgggtct
ctctggttagaccagatctgagcctgggagctctctggctaactagggaacccactgcttaagcctcaataaagcttgccttgagtgcttcaa
gtagtgtgtgcccgtctgttgtgtgactctggtaactagagatccctcagacccttttagtcagtgtggaaaatctctagcagtggcgcccgaa
cagggacttgaaagcgaaagggaaaccagaggagctctctcgacgcaggactcggcttgctgaagcgcgcacggcaagaggcgagg
ggcggcgactggtgagtacgccaaaaattttgactagcggaggctagaaggagagagatgggtgcgagagcgtcagtattaagcgggg
gagaattagatcgcgatgggaaaaaattcggttaaggccagggggaaagaaaaaatataaattaaaacatatagtatgggcaagcaggga
gctagaacgattcgcagttaatcctggcctgttagaaacatcagaaggctgtagacaaatactgggacagctacaaccatcccttcagacag
gatcagaagaacttagatcattatataatacagtagcaaccctctattgtgtgcatcaaaggatagagataaaagacaccaaggaagctttag
acaagatagaggaagagcaaaacaaaagtaagaccaccgcacagcaagcggccgctgatcttcagacctggaggaggagatatgagg
gacaattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatctgttgca
actcacagtctggggcatcaagcagctccaggcaagaatcctggctgtggaaagatacctaaaggatcaacagctcctggggatttgggg
ttgctctggaaaactcatttgcaccactgctgtgccttggaatgctagttggagtaataaatctctggaacagatttggaatcacacgacctgg
atggagtgggacagagaaattaacaattacacaagcttaatacactccttaattgaagaatcgcaaaaccagcaagaaaagaatgaacaag
aattattggaattagataaatgggcaagtttgtggaattggtttaacataacaaattggctgtggtatataaaattattcataatgatagtaggag
gcttggtaggtttaagaatagtttttgctgtactttctatagtgaatagagttaggcagggatattcaccattatcgtttcagacccacctcccaac
cccgaggggacccgacaggcccgaaggaatagaagaagaaggtggagagagagacagagacagatccattcgattagtgaacggatc
ggcactgcgtgcgccaattctgcagacaaatggcagtattcatccacaattttaaaagaaaaggggggattggggggtacagtgcagggg
aaagaatagtagacataatagcaacagacatacaaactaaagaattacaaaaacaaattacaaaaattcaaaattttcgggtttattacaggg
acagcagagatccagtttggttaattaatggggggacgttaacggggcggaacggtaccgagggcctatttcccatgattccttcatatttg
catatacgatacaaggctgttagagagataattagaattaatttgactgtaaacacaaagatattagtacaaaatacgtgacgtagaaagtaataat
ttcttgggtagtttgcagttttaaaattatgttttaaaatggactatcatatgcttaccgtaacttgaaagtatttcgatttcttggctttatatat
cttgtggaaaggacgaaacaccggagacgtgtacacgtctctgttttagagctagaaatagcaagttaaaataaggctagtccgttatcaacttg
aaaaagtggcaccgagtcggtgcttttttgaattcgctagctaggtcttgaaaggagtgggaattggctccggtgcccgtcagtgggcaga
gcgcacatcgcccacagtccccgagaagttggggggaggggtcggcaattgatccggtgcctagagaaggtggcgcggggtaaactg
ggaaagtgatgtcgtgtactggctccgcctttttcccgaggggggggagaaccgtatataagtgcagtagtcgccgtgaacgttctttttcg
caacgggtttgccgccagaacacaggaccggtgccaccatggactataaggaccacgacggagactacaaggatcatgatattgattaca
aagacgatgacgataagatggccccaaagaagaagcggaaggtcggtatccacggagtcccagcagccgacaagaagtacagcatcg
gcctggccatcggcaccaactctgtgggctgggccgtgatcaccgacgagtacaaggtgcccagcaagaaattcaaggtgctgggcaac
accgaccggcacagcatcaagaagaacctgatcggagccctgctgttcgacagcggcgaaacagccgaggccacccggctgaagaga
accgccagaagaagatacaccagacggaagaaccggatctgctatctgcaagagatcttcagcaacgagatggccaaggtggacgaca
gcttcttccacagactggaagagtccttcctggtggaagaggataagaagcacgagcggcaccccatcttcggcaacatcgtggacgagg
tggcctaccacgagaagtaccccaccatctaccacctgagaaagaaactggtggacagcaccgacaaggccgacctgcggctgatctat
ctggccctggcccacatgatcaagttccggggccacttcctgatcgagggcgacctgaaccccgacaacagcgacgtggacaagctgtt
catccagctggtgcagacctacaaccagctgttcgaggaaaaccccatcaacgccagcggcgtggacgccaaggccatcctgtctgcca
gactgagcaagagcagacggctggaaaatctgatcgcccagctgcccggcgagaagaagaatggcctgttcggcaacctgattgccctg
agcctgggcctgacccccaacttcaagagcaacttcgacctggccgaggatgccaaactgcagctgagcaaggacacctacgacgacg
acctggacaacctgctggcccagatcggcgaccagtacgccgacctgtttctggccgccaagaacctgtccgacgccatcctgctgagcg
acatcctgagagtgaacaccgagatcaccaaggcccccctgagcgcctctatgatcaagagatacgacgagcaccaccaggacctgacc
ctgctgaaagctctcgtgcggcagcagctgcctgagaagtacaaagagattttcttcgaccagagcaagaacggctacgccggctacattg
acggcggagccagccaggaagagttctacaagttcatcaagcccatcctggaaaagatggacggcaccgaggaactgctcgtgaagct
gaacagagaggacctgctgcggaagcagcggaccttcgacaacggcagcatcccccaccagatccacctgggagagctgcacgccatt
ctgcggcggcaggaagatttttacccattcctgaaggacaaccgggaaaagatcgagaagatcctgaccttccgcatcccctactacgtgg
gccctctggccaggggaaacagcagattcgcctggatgaccagaaagagcgaggaaaccatcaccccctggaacttcgaggaagtggt
ggacaagggcgcttccgcccagagcttcategagcggatgaccaacttcgataagaacctgcccaacgagaaggtgctgcccaagcac
agcctgctgtacgagtacttcaccgtgtataacgagctgaccaaagtgaaatacgtgaccgagggaatgagaaagcccgccttcctgagc
ggcgagcagaaaaaggccatcgtggacctgctgttcaagaccaaccggaaagtgaccgtgaagcagctgaaagaggactacttcaaga
aaatcgagtgcttcgactccgtggaaatctccggcgtggaagatcggttcaacgcctccctgggcacataccacgatctgctgaaaattatc
aaggacaaggacttcctggacaatgaggaaaacgaggacattctggaagatatcgtgctgaccctgacactgtttgaggacagagagatg
atcgaggaacggctgaaaacctatgcccacctgttcgacgacaaagtgatgaagcagctgaagcggcggagatacaccggctggggca
ggctgagccggaagctgatcaacggcatccgggacaagcagtccggcaagacaatcctggatttcctgaagtccgacggcttcgccaac
agaaacttcatgcagctgatccacgacgacagcctgacctttaaagaggacatccagaaagcccaggtgtccggccagggcgatagcct
gcacgagcacattgccaatctggccggcagccccgccattaagaagggcatcctgcagacagtgaaggtggtggacgagctcgtgaaa
gtgatgggccggcacaagcccgagaacatcgtgatcgaaatggccagagagaaccagaccacccagaagggacagaagaacagccg
cgagagaatgaagcggatcgaagagggcatcaaagagctgggcagccagatcctgaaagaacaccccgtggaaaacacccagctgca
gaacgagaagctgtacctgtactacctgcagaatggggggatatgtacgtggaccaggaactggacatcaaccggctgtccgactacga
tgtggacgctatcgtgcctcagagctttctgaaggacgactccatcgacaacaaggtgctgaccagaagcgacaagaaccggggcaaga
gcgacaacgtgccctccgaagaggtcgtgaagaagatgaagaactactggcggcagctgctgaacgccaagctgattacccagagaaa
gttcgacaatctgaccaaggccgagagaggcggcctgagcgaactggataaggccggcttcatcaagagacagctggtggaaacccgg
cagatcacaaagcacgtggcacagatcctggactcccggatgaacactaagtacgacgagaatgacaagctgatccgggaagtgaaagt
gatcaccctgaagtccaagctggtgtccgatttccggaaggatttccagttttacaaagtgcgcgagatcaacaactaccaccacgcccacg
acgcctacctgaacgccgtcgtgggaaccgccctgatcaaaaagtaccctaagctggaaagcgagttcgtgtacggcgactacaaggtgt
acgacgtgcggaagatgatcgccaagagcgagcaggaaatcggcaaggctaccgccaagtacttcttctacagcaacatcatgaacttttt
caagaccgagattaccctggccaacggcgagatccggaagcggcctctgatcgagacaaacggcgaaaccggggagatcgtgtggga
taagggccgggattttgccaccgtgcggaaagtgctgagcatgccccaagtgaatatcgtgaaaaagaccgaggtgcagacaggcggct
tcagcaaagagtctatcctgcccaagaggaacagcgataagctgatcgccagaaagaaggactgggaccctaagaagtacggcggcttc
gacagccccaccgtggcctattctgtgctggtggtggccaaagtggaaaagggcaagtccaagaaactgaagagtgtgaaagagctgct
ggggatcaccatcatggaaagaagcagcttcgagaagaatcccatcgactttctggaagccaagggctacaaagaagtgaaaaaggacc
tgatcatcaagctgcctaagtactccctgttcgagctggaaaacggccggaagagaatgctggcctctgccggcgaactgcagaagggaa
acgaactggccctgccctccaaatatgtgaacttcctgtacctggccagccactatgagaagctgaagggctcccccgaggataatgagca
gaaacagctgtttgtggaacagcacaagcactacctggacgagatcatcgagcagatcagcgagttctccaagagagtgatcctggccga
cgctaatctggacaaagtgctgtccgcctacaacaagcaccgggataagcccatcagagagcaggccgagaatatcatccacctgtttac
cctgaccaatctgggagcccctgccgccttcaagtactttgacaccaccatcgaccggaagaggtacaccagcaccaaagaggtgctgg
acgccaccctgatccaccagagcatcaccggcctgtacgagacacggatcgacctgtctcagctgggaggcgacaaaaggccggcgg
ccacgaaaaaggccggacaggccaaaaagaaaaagctcgagggggaggcgggagcggatccccctcccggctccagatgttcttcg
ctaataaccacgaccaggaatttgaccctccaaaggtttacccacctgtcccagctgagaagaggaagcccatccgggtgctgtctctcttt
gatggaatcgctacagggctcctggtgctgaaggacttgggcattcaggtggaccgctacattgcctcggaggtgtgtgaggactccatca
cggtgggcatggtgcggcaccaggggaagatcatgtacgtcggggacgtccgcagcgtcacacagaagcatatccaggagtggggcc
cattcgatctggtgattgggggcagtccctgcaatgacctctccatcgtcaaccctgctcgcaagggcctctacgagggcactggccggct
cttctttgagttctaccgcctcctgcatgatgcgcggcccaaggagggagatgatcgccccttcttctggctctttgagaatgtggtggccatg
ggcgttagtgacaagagggacatctcgcgatttctcgagtccaaccctgtgatgattgatgccaaagaagtgtcagctgcacacagggccc
gctacttctggggtaaccttcccggtatgaacaggccgttggcatccactgtgaatgataagctggagctgcaggagtgtctggagcatgg
caggatagccaagttcagcaaagtgaggaccattactacgaggtcaaactccataaagcagggcaaagaccagcattttcctgtgttcatg
aatgagaaagaggacatcttatggtgcactgaaatggaaagggtatttggtttcccagtccactatactgacgtgtccaacatgagccgcttg
gcgaggcagagactgctgggccggtcatggagcgtgccagtcatccgccacctcttcgctccgctgaaggagtattttgcgtgtgtgtccg
gccggggccggcccggatccggcgcaacaaacttctctctgctgaaacaagccggagatgtcgaagagaatcctggaccgatggtgag
caagggcgaggagctgttcaccggggtggtgcccatcctggtcgagctggacggcgacgtaaacggccacaagttcagcgtgtccggc
gagggcgagggcgatgccacctacggcaagctgaccctgaagttcatctgcaccaccggcaagctgcccgtgccctggcccaccctcgt
gaccaccctgacctacggcgtgcagtgcttcagccgctaccccgaccacatgaagcagcacgacttcttcaagtccgccatgcccgaagg
ctacgtccaggagcgcaccatcttcttcaaggacgacggcaactacaagacccgcgccgaggtgaagttcgagggcgacaccctggtga
accgcatcgagctgaagggcatcgacttcaaggaggacggcaacatcctggggcacaagctggagtacaactacaacagccacaacgt
ctatatcatggccgacaagcagaagaacggcatcaaggtgaacttcaagatccgccacaacatcgaggacggcagcgtgcagctcgcc
gaccactaccagcagaacacccccatcggcgacggccccgtgctgctgcccgacaaccactacctgagcacccagtccgccctgagca
aagaccccaacgagaagcgcgatcacatggtcctgctggagttcgtgaccgccgccgggatcactctcggcatggacgagctgtacaag
taaagcggccgcgtcgacaatcaacctctggattacaaaatttgtgaaagattgactggtattcttaactatgttgctccttttacgctatgtgga
tacgctgctttaatgcctttgtatcatgctattgcttcccgtatggctttcattttctcctccttgtataaatcctggttgctgtctctttatgagga
gttgtggcccgttgtcaggcaacgtggcgtggtgtgcactgtgtttgctgacgcaacccccactggttggggcattgccaccacctgtcagctc
ctttccgggactttcgctttccccctccctattgccacggeggaactcatcgccgcctgccttgcccgctgctggacaggggctcggctgttg
ggcactgacaattccgtggtgttgtcggggaagctgacgtcctttccatggctgctcgcctgtgttgccacctggattctgcgcgggacgtc
cttctgctacgtcccttcggccctcaatccagcggaccttccttcccgcggcctgctgccggctctgcggcctettccgcgtcttegccttcgc
cctcagacgagtcggatctccctttgggccgcctccccgcctggaattcgagctcggtacctttaagaccaatgacttacaaggcagctgta
gatcttagccactttttaaaagaaaaggggggactggaagggctaattcactcccaacgaagacaagatctgctttttgcttgtactgggtctc
tctggttagaccagatctgagcctgggagctctctggctaactagggaacccactgcttaagcctcaataaagcttgccttgagtgcttcaag
tagtgtgtgcccgtctgttgtgtgactctggtaactagagatccctcagacccttttagtcagtgtggaaaatctctagcagtagtagttcatgtc
atcttattattcagtatttataacttgcaaagaaatgaatatcagagagtgagaggaacttgtttattgcagcttataatggttacaaataaagcaa
tagcatcacaaatttcacaaataaagcatttttttcactgcattctagttgtggtttgtccaaactcatcaatgtatcttatcatgtctggctctagc
tatcccgcccctaactccgcccatcccgcccctaactccgcccagttccgcccattctccgccccatggctgactaattttttttatttatgcagag
gccgaggccgcctcggcctctgagctattccagaagtagtgaggaggcttttttggaggcctagggacgtacccaattcgccctatagtga
gtcgtattacgcgcgctcactggccgtcgttttacaacgtcgtgactgggaaaaccctggcgttacccaacttaatcgccttgcagcacatcc
ccctttcgccagctggcgtaatagcgaagaggcccgcaccgatcgcccttcccaacagttgcgcagcctgaatggcgaatgggacgcgc
cctgtagcggcgcattaagcgcggcgggtgtggtggttacgcgcagcgtgaccgctacacttgccagcgccctagegcccgctcctttcg
ctttcttcccttcctttctcgccacgttcgccggctttccccgtcaagctctaaatcgggggctccctttagggttccgatttagtgctttacggca
cctcgaccccaaaaaacttgattagggtgatggttcacgtagtgggccatcgccctgatagacggtttttcgccctttgacgttggagtccacgt
tctttaatagtggactcttgttccaaactggaacaacactcaaccctatctcggtctattcttttgatttataagggattttgccgatttcggccta
ttggttaaaaaatgagctgatttaacaaaaatttaacgcgaattttaacaaaatattaacgcttacaatttaggtgccggccatgaccgagatcg
gcgagcagccgtggggggggagttcgccctgcgcgacccggccggcaactgcgtgcacttcgtggccgaggagcaggactgacac
gtgctacgagatttcgattccaccgccgccttctatgaaaggttgggcttcggaatcgttttccgggacgccggctggatgatcctccagcg
cggggatctcatgctggagttcttcgcccaccccaacttgtttattgcagcttataatggttacaaataaagcaatagcatcacaaatttcacaa
ataaagcatttttttcactgcattctagttgtggtttgtccaaactcatcaatgtatcttatcatgtctgtataccgtcgacctctagctagagct
tggcgtaatcatggtcatagctgtttcctgtgtgaaattgttatccgctcacaattccacacaacatacgagccggaagcataaagtgtaaagcctg
gggtgcctaatgagtgagctaactcacattaattgcgttgcgctcactgcccgctttccagtcgggaaacctgtcgtgccagctgcattaatg
aatcggccaacgcgcggggagaggcggtttgcgtattgggcgctcttccgcttcctcgctcactgactcgctgcgctcggtcgttcggctg
cggcgagcggtatcagctcactcaaaggcggtaatacggttatccacagaatcaggggataacgcaggaaagaacatgtgagcaaaag
gccagcaaaaggccaggaaccgtaaaaaggccgcgttgctggcgtttttccataggctccgcccccctgacgagcatcacaaaaatcga
cgctcaagtcagaggtggcgaaacccgacaggactataaagataccaggcgtttccccctggaagctccctcgtgcgctctcctgttccga
ccctgccgcttaccggatacctgtccgcctttctcccttcgggaagcgtggcgctttctcatagctcacgctgtaggtatctcagttcggtgta
ggtcgttcgctccaagctgggctgtgtgcacgaaccccccgttcagcccgaccgctgcgccttatccggtaactategtcttgagtccaacc
cggtaagacacgacttatcgccactggcagcagccactggtaacaggattagcagagcgaggtatgtaggcggtgctacagagttcttga
agtggtggcctaactacggctacactagaagaacagtatttggtatctgcgctctgctgaagccagttaccttcggaaaaagagttggtagct
cttgatccggcaaacaaaccaccgctggtagcggtggtttttttgtttgcaagcagcagattacgcgcagaaaaaaaggatctcaagaagat
cctttgatcttttctacggggtctgacgctcagtggaacgaaaactcacgttaagggattttggtcatgagattatcaaaaaggatcttcaccta
gatccttttaaattaaaaatgaagttttaaatcaatctaaagtatatatgagtaaacttggtctgacagttaccaatgcttaatcagtgaggcacct
atctcagcgatctgtctatttcgttcatccatagttgcctgactccccgtcgtgtagataactacgatacgggagggcttaccatctggcccca
gtgctgcaatgataccgcgagacccacgctcaccggctccagatttatcagcaataaaccagccagccggaagggccgagcgcagaag
tggtcctgcaactttatccgcctccatccagtctattaattgttgccgggaagctagagtaagtagttcgccagttaatagtttgcgcaacgttgt
tgccattgctacaggcatcgtggtgtcacgctcgtcgtttggtatggcttcattcagctccggttcccaacgatcaaggcgagttacatgatcc
cccatgttgtgcaaaaaagcggttagctccttcggtcctccgatcgttgtcagaagtaagttggccgcagtgttatcactcatggttatggcag
cactgcataattctcttactgtcatgccatccgtaagatgcttttctgtgactggtgagtactcaaccaagtcattctgagaatagtgtatgcggc
gaccgagttgctcttgcccggcgtcaatacgggataataccgcgccacatagcagaactttaaaagtgctcatcattggaaaacgttcttcg
gggcgaaaactctcaaggatcttaccgctgttgagatccagttcgatgtaacccactcgtgcacccaactgatcttcagcatcttttactttcac
cagcgtttctgggtgagcaaaaacaggaaggcaaaatgccgcaaaaaagggaataagggcgacacggaaatgttgaatactcatactctt
cctttttcaatattattgaagcatttatcagggttattgtctcatgagcggatacatatttgaatgtatttagaaaaataaacaaataggggttccgc
gcacatttccccgaaaagtgccacctgac.

In an aspect, a disclosed pBK500 plasmid can comprise the sequence set forth below or a sequence having 70%, 75%, 80%, 85%, 90%, 95%, or more identity to the sequence set forth

(SEQ ID NO: 23)
gtcgacggatcgggagatctcccgatcccctatggtgcactctcagtacaatctgctctgatgccgcatagttaagccagtatctgctccctg
cttgtgtgttggaggtcgctgagtagtgcgcgagcaaaatttaagctacaacaaggcaaggcttgaccgacaattgcatgaagaatctgctt
agggttaggcgttttgcgctgcttcgcgatgtacgggccagatatacgcgttgacattgattattgactagttattaatagtaatcaattacggg
gtcattagttcatagcccatatatggagttccgcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtacatcaatgggcgtggat
agcggtttgactcacggggatttccaagtctccaccccattgacgtcaatgggagtttgttttggcaccaaaatcaacgggactttccaaaatgt
cgtaacaactccgccccattgacgcaaatgggggtaggcgtgtacggtgggaggtctatataagcagcgcgttttgcctgtactgggtct
ctctggttagaccagatctgagcctgggagctctctggctaactagggaacccactgcttaagcctcaataaagcttgccttgagtgcttcaa
gtagtgtgtgcccgtctgttgtgtgactctggtaactagagatccctcagacccttttagtcagtgtggaaaatctctagcagtggcgcccgaa
cagggacttgaaagcgaaagggaaaccagaggagctctctcgacgcaggactcggcttgctgaagcgcgcacggcaagaggcgagg
ggcggcgactggtgagtacgccaaaaattttgactagcggaggctagaaggagagagatgggtgcgagagcgtcagtattaagcgggg
gagaattagatcgcgatgggaaaaaattcggttaaggccagggggaaagaaaaaatataaattaaaacatatagtatgggcaagcaggga
gctagaacgattcgcagttaatcctggcctgttagaaacatcagaaggctgtagacaaatactgggacagctacaaccatcccttcagacag
gatcagaagaacttagatcattatataatacagtagcaaccctctattgtgtgcatcaaaggatagagataaaagacaccaaggaagctttag
acaagatagaggaagagcaaaacaaaagtaagaccaccgcacagcaagcggccgctgatcttcagacctggaggaggagatatgagg
gacaattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatctgttgca
actcacagtctggggcatcaagcagctccaggcaagaatcctggctgtggaaagatacctaaaggatcaacagctcctggggatttgggg
ttgctctggaaaactcatttgcaccactgctgtgccttggaatgctagttggagtaataaatctctggaacagatttggaatcacacgacctgg
atggagtgggacagagaaattaacaattacacaagcttaatacactccttaattgaagaatcgcaaaaccagcaagaaaagaatgaacaaga
attattggaattagataaatgggcaagtttgtggaattggtttaacataacaaattggctgtggtatataaaattattcataatgatagtaggag
gcttggtaggtttaagaatagtttttgctgtactttctatagtgaatagagttaggcagggatattcaccattatcgtttcagacccacctccca
accccgaggggacccgacaggcccgaaggaatagaagaagaaggtggagagagagacagagacagatccattcgattagtgaacggatc
ggcactgcgtgcgccaattctgcagacaaatggcagtattcatccacaattttaaaagaaaaggggggattggggggtacagtgcagggg
aaagaatagtagacataatagcaacagacatacaaactaaagaattacaaaaacaaattacaaaaattcaaaattttcgggtttattacaggg
acagcagagatccagtttggttaattaatggggggacgttaacggggcggaacggtaccgagggcctatttcccatgattccttcatatttgc
atatacgatacaaggctgttagagagataattagaattaatttgactgtaaacacaaagatattagtacaaaatacgtgacgtagaaagtaataa
tttcttgggtagtttgcagttttaaaattatgttttaaaatggactatcatatgcttaccgtaacttgaaagtatttcgatttcttggctttata
tatcttgtggaaaggacgaaacaccgctgctcagggtagatagctggttttagagctagaaatagcaagttaaaataaggctagtccgttatcaa
cttgaaaaagtggcaccgagtcggtgcttttttgaattcgctagctaggtcttgaaaggagtgggaattggctccggtgcccgtcagtgggcag
agcgcacatcgcccacagtccccgagaagttggggggaggggtcggcaattgatccggtgcctagagaaggtggcgcggggtaaact
gggaaagtgatgtcgtgtactggctccgcctttttcccgagggtgggggagaaccgtatataagtgcagtagtcgccgtgaacgttctttttc
gcaacgggtttgccgccagaacacaggaccggttctagagcgctgccaccatggacaagaagtacagcatcggcctggacatcggcac
caactctgtgggctgggccgtgatcaccgacgagtacaaggtgcccagcaagaaattcaaggtgctgggcaacaccgaccggcacagc
atcaagaagaacctgatcggagccctgctgttcgacagcggcgaaacagccgaggccacccggctgaagagaaccgccagaagaaga
tacaccagacggaagaaccggatctgctatctgcaagagatcttcagcaacgagatggccaaggtggacgacagcttcttccacagactg
gaagagtccttcctggtggaagaggataagaagcacgagcggcaccccatcttcggcaacatcgtggacgaggtggcctaccacgaga
agtaccccaccatctaccacctgagaaagaaactggtggacagcaccgacaaggccgacctgcggctgatctatctggccctggcccac
atgatcaagttccggggccacttcctgatcgagggcgacctgaaccccgacaacagcgacgtggacaagctgttcatccagctggtgcag
acctacaaccagctgttcgaggaaaaccccatcaacgccagcggcgtggacgccaaggccatcctgtctgccagactgagcaagagca
gacggctggaaaatctgatcgcccagctgcccggcgagaagaagaatggcctgttcggaaacctgattgccctgagcctgggcctgacc
cccaacttcaagagcaacttcgacctggccgaggatgccaaactgcagctgagcaaggacacctacgacgacgacctggacaacctgct
ggcccagatcggcgaccagtacgccgacctgtttctggccgccaagaacctgtccgacgccatcctgctgagcgacatcctgagagtga
acaccgagatcaccaaggcccccctgagcgcctctatgatcaagagatacgacgagcaccaccaggacctgaccctgctgaaagctctc
gtgcggcagcagctgcctgagaagtacaaagagattttcttcgaccagagcaagaacggctacgccggctacattgacggcggagccag
ccaggaagagttctacaagttcatcaagcccatcctggaaaagatggacggcaccgaggaactgctcgtgaagctgaacagagaggacc
tgctgcggaagcagcggaccttcgacaacggcagcatcccccaccagatccacctgggagagctgcacgccattctgcggcggcagga
agatttttacccattcctgaaggacaaccgggaaaagatcgagaagatcctgaccttccgcatcccctactacgtgggccctctggccagg
ggaaacagcagattcgcctggatgaccagaaagagcgaggaaaccatcaccccctggaacttcgaggaagtggtggacaagggcgctt
ccgcccagagcttcatcgagcggatgaccaacttcgataagaacctgcccaacgagaaggtgctgcccaagcacagcctgctgtacgag
tacttcaccgtgtataacgagctgaccaaagtgaaatacgtgaccgagggaatgagaaagcccgccttcctgagcggcgagcagaaaaa
ggccatcgtggacctgctgttcaagaccaaccggaaagtgaccgtgaagcagctgaaagaggactacttcaagaaaatcgagtgcttcga
ctccgtggaaatctccggcgtggaagatcggttcaacgcctccctgggcacataccacgatctgctgaaaattatcaaggacaaggacttc
ctggacaatgaggaaaacgaggacattctggaagatatcgtgctgaccctgacactgtttgaggacagagagatgatcgaggaacggctg
aaaacctatgcccacctgttcgacgacaaagtgatgaagcagctgaagcggcggagatacaccggctggggcaggctgagccggaag
ctgatcaacggcatccgggacaagcagtccggcaagacaatcctggatttcctgaagtccgacggcttcgccaacagaaacttcatgcag
ctgatccacgacgacagcctgacctttaaagaggacatccagaaagcccaggtgtccggccagggcgatagcctgcacgagcacattgc
caatctggccggcagccccgccattaagaagggcatcctgcagacagtgaaggtggtggacgagctcgtgaaagtgatgggccggcac
aagcccgagaacatcgtgatcgaaatggccagagagaaccagaccacccagaagggacagaagaacagccgcgagagaatgaagcg
gatcgaagagggcatcaaagagctgggcagccagatcctgaaagaacaccccgtggaaaacacccagctgcagaacgagaagctgta
cctgtactacctgcagaatggggggatatgtacgtggaccaggaactggacatcaaccggctgtccgactacgatgtggaccatatcgtg
cctcagagctttctgaaggacgactccatcgacaacaaggtgctgaccagaagcgacaagaaccggggcaagagcgacaacgtgccct
ccgaagaggtcgtgaagaagatgaagaactactggcggcagctgctgaacgccaagctgattacccagagaaagttcgacaatctgacc
aaggccgagagaggcggcctgagcgaactggataaggccggcttcatcaagagacagctggtggaaacccggcagatcacaaagcac
gtggcacagatcctggactcccggatgaacactaagtacgacgagaatgacaagctgatccgggaagtgaaagtgatcaccctgaagtc
caagctggtgtccgatttccggaaggatttccagttttacaaagtgcgcgagatcaacaactaccaccacgcccacgacgcctacctgaac
gccgtcgtgggaaccgccctgatcaaaaagtaccctaagctggaaagcgagttcgtgtacggcgactacaaggtgtacgacgtgcggaa
gatgatcgccaagagcgagcaggaaatcggcaaggctaccgccaagtacttcttctacagcaacatcatgaactttttcaagaccgagatta
ccctggccaacggcgagatccggaagcggcctctgatcgagacaaacggcgaaaccggggagatcgtgtgggataagggccgggatt
ttgccaccgtgcggaaagtgctgagcatgccccaagtgaatatcgtgaaaaagaccgaggtgcagacaggcggcttcagcaaagagtct
atcctgcccaagaggaacagcgataagctgatcgccagaaagaaggactgggaccctaagaagtacggcggcttcgacagccccaccg
tggcctattctgtgctggtggtggccaaagtggaaaagggcaagtccaagaaactgaagagtgtgaaagagctgctggggatcaccatca
tggaaagaagcagcttcgagaagaatcccatcgactttctggaagccaagggctacaaagaagtgaaaaaggacctgatcatcaagctgc
ctaagtactccctgttcgagctggaaaacggccggaagagaatgctggcctctgccggcgaactgcagaagggaaacgaactggccctg
ccctccaaatatgtgaacttcctgtacctggccagccactatgagaagctgaagggctcccccgaggataatgagcagaaacagctgtttgt
ggaacagcacaagcactacctggacgagatcatcgagcagatcagcgagttctccaagagagtgatcctggccgacgctaatctggaca
aagtgctgtccgcctacaacaagcaccgggataagcccatcagagagcaggccgagaatatcatccacctgtttaccctgaccaatctgg
gagcccctgccgccttcaagtactttgacaccaccatcgaccggaagaggtacaccagcaccaaagaggtgctggacgccaccctgatc
caccagagcatcaccggcctgtacgagacacggatcgacctgtctcagctgggaggcgacaagcgacctgccgccacaaagaaggct
ggacaggctaagaagaagaaagattacaaagacgatgacgataagggatccggcgcaacaaacttctctctgctgaaacaagccggag
atgtcgaagagaatcctggaccgaccgagtacaagcccacggtgcgcctcgccacccgcgacgacgtccccagggccgtacgcaccct
cgccgccgcgttcgccgactaccccgccacgcgccacacegtcgatccggaccgccacatcgagcgggtcaccgagctgcaagaact
cttcctcacgcgcgtcgggctcgacatcggcaaggtgtgggtcgcggacgacggcgccgcggtggcggtctggaccacgccggagag
cgtcgaagcgggggcggtgttcgccgagatcggcccgcgcatggccgagttgagcggttcccggctggccgcgcagcaacagatgga
aggcctcctggcgccgcaccggcccaaggagcccgcgtggttcctggccaccgtcggagtctcgcccgaccaccagggcaagggtct
gggcagcgccgtcgtgctccccggagtggaggcggccgagcgcgccggggtgcccgccttcctggagacctccgcgccccgcaacct
ccccttctacgagcggctcggcttcaccgtcaccgccgacgtcgaggtgcccgaaggaccgcgcacctggtgcatgaccegcaagcccggt
gcctgaacgcgttaagtcgacaatcaacctctggattacaaaatttgtgaaagattgactggtattcttaactatgttgctccttttacgctat
gtggatacgctgctttaatgcctttgtatcatgctattgcttcccgtatggctttcattttctcctccttgtataaatcctggttgctgtctct
ttatgaggagttgtggcccgttgtcaggcaacgtggcgtggtgtgcactgtgtttgctgacgcaacccccactggttggggcattgccaccacc
tgtcagctcctttccgggactttcgctttccccctccctattgccacggcggaactcatcgccgcctgccttgcccgctgctggacaggggctcg
gctgttgggcactgacaattccgtggtgttgtcggggaaatcatcgtcctttccttggctgctcgcctgtgttgccacctggattctgcgcggga
cgtccttctgctacgtcccttcggccctcaatccagcggaccttccttcccgcggcctgctgccggctctgcggcctcttcegcgtcttcgcctt
cgccctcagacgagtcggatctccctttgggccgcctccccgcgtcgactttaagaccaatgacttacaaggcagctgtagatcttagccactt
tttaaaagaaaaggggggactggaagggctaattcactcccaacgaagacaagatctgctttttgcttgtactgggtctctctggttagacca
gatctgagcctgggagctctctggctaactagggaacccactgcttaagcctcaataaagcttgccttgagtgcttcaagtagtgtgtgcccg
tctgttgtgtgactctggtaactagagatccctcagacccttttagtcagtgtggaaaatctctagcagggcccgtttaaacccgctgatcagc
ctcgactgtgccttctagttgccagccatctgttgtttgcccctcccccgtgccttccttgaccctggaaggtgccactcccactgtcctttcct
aataaaatgaggaaattgcatcgcattgtctgagtaggtgtcattctattctggggggtggggggggcaggacagcaagggggaggattg
ggaagacaatagcaggcatgctggggatgcggtgggctctatggcttctgaggcggaaagaaccagctggggctctagggggtatccc
cacgcgccctgtagcggcgcattaagcgcggcgggtgtggtggttacgcgcagcgtgaccgctacacttgccagcgccctagcgcccgctc
ctttcgctttcttcccttcctttctcgccacgttcgccggctttccccgtcaagctctaaatcgggggctccctttagggttccgatttagtgct
ttacggcacctcgaccccaaaaaacttgattagggtgatggttcacgtagtgggccatcgccctgatagacggtttttcgccctttgacgttg
gagtccacgttctttaatagtggactcttgttccaaactggaacaacactcaaccctatctcggtctattcttttgatttataagggattttgcc
gatttcggcctattggttaaaaaatgagctgatttaacaaaaatttaacgcgaattaattctgtggaatgtgtgtcagttagggtgtggaaagtc
cccaggctccccagcaggcagaagtatgcaaagcatgcatctcaattagtcagcaaccaggtgtggaaagtccccaggctccccagcaggca
gaagtatgcaaagcatgcatctcaattagtcagcaaccatagtcccgcccctaactccgcccatcccgcccctaactccgcccagttcegcc
cattctccgccccatggctgactaattttttttatttatgcagaggccgaggccgcctctgcctctgagctattccagaagtagtgaggaggctt
ttttggaggcctaggcttttgcaaaaagctcccgggagcttgtatatccattttcggatctgatcagcacgtgttgacaattaatcatcggcata
gtatatcggcatagtataatacgacaaggtgaggaactaaaccatggccaagttgaccagtgccgttccggtgctcaccgcgcgcgacgtc
gccggagcggtcgagttctggaccgaccggctcgggttctcccgggacttcgtggaggacgacttcgccggtgtggtccgggacgacgt
gaccctgttcatcagcgcggtccaggaccaggtggtgccggacaacaccctggcctgggtgtgggtgcgcggcctggacgagctgtac
gccgagtggtcggaggtcgtgtccacgaacttccgggacgcctccgggccggccatgaccgagatcggcgagcagccgtgggggcg
ggagttcgccctgcgcgacccggccggcaactgcgtgcacttcgtggccgaggagcaggactgacacgtgctacgagatttcgattcca
ccgccgccttctatgaaaggttgggcttcggaatcgttttccgggacgccggctggatgatcctccagcgcggggatctcatgctggagttcttc
gcccaccccaacttgtttattgcagcttataatggttacaaataaagcaatagcatcacaaatttcacaaataaagcatttttttcactgcattc
tagttgtggtttgtccaaactcatcaatgtatcttatcatgtctgtataccgtcgacctctagctagagcttggcgtaatcatggtcatagctgt
ttcctgtgtgaaattgttatccgctcacaattccacacaacatacgagccggaagcataaagtgtaaagcctggggtgcctaatgagtgagctaa
ctcacattaattgcgttgcgctcactgcccgctttccagtcgggaaacctgtcgtgccagctgcattaatgaatcggccaacgcgcggggag
aggcggtttgcgtattgggcgctcttccgcttcctcgctcactgactcgctgcgctcggtcgttcggctgcggcgagcggtatcagctcactc
aaaggcggtaatacggttatccacagaatcaggggataacgcaggaaagaacatgtgagcaaaaggccagcaaaaggccaggaaccg
taaaaaggccgcgttgctggcgtttttccataggctccgcccccctgacgagcatcacaaaaatcgacgctcaagtcagaggtggcgaaa
cccgacaggactataaagataccaggcgtttccccctggaagctccctcgtgcgctctcctgttccgaccctgccgcttaccggatacctgt
ccgcctttctcccttcgggaagcgtggcgctttctcatagctcacgctgtaggtatctcagttcggtgtaggtcgttcgctccaagctgggctg
tgtgcacgaaccccccgttcagcccgaccgctgcgccttatccggtaactatcgtcttgagtccaacccggtaagacacgacttatcgccac
tggcagcagccactggtaacaggattagcagagcgaggtatgtaggcggtgctacagagttcttgaagtggtggcctaactacggctaca
ctagaagaacagtatttggtatctgcgctctgctgaagccagttaccttcggaaaaagagttggtagctcttgatccggcaaacaaaccacc
gctggtagcggtggtttttttgtttgcaagcagcagattacgcgcagaaaaaaaggatctcaagaagatcctttgatcttttctacggggtctg
acgctcagtggaacgaaaactcacgttaagggattttggtcatgagattatcaaaaaggatcttcacctagatccttttaaattaaaaatgaagt
tttaaatcaatctaaagtatatatgagtaaacttggtctgacagttaccaatgcttaatcagtgaggcacctatctcagcgatctgtctatttcg
ttcatccatagttgcctgactccccgtcgtgtagataactacgatacgggagggcttaccatctggccccagtgctgcaatgataccgcgagacc
cacgctcaccggctccagatttatcagcaataaaccagccagccggaagggccgagcgcagaagtggtcctgcaactttatccgcctcca
tccagtctattaattgttgccgggaagctagagtaagtagttcgccagttaatagtttgcgcaacgttgttgccattgctacaggcatcgtggtg
tcacgctcgtcgtttggtatggcttcattcagctccggttcccaacgatcaaggcgagttacatgatcccccatgttgtgcaaaaaagcggtta
gctccttcggtcctccgatcgttgtcagaagtaagttggccgcagtgttatcactcatggttatggcagcactgcataattctcttactgtcatg
ccatccgtaagatgcttttctgtgactggtgagtactcaaccaagtcattctgagaatagtgtatgcggcgaccgagttgctcttgcccggcgtc
aatacgggataataccgcgccacatagcagaactttaaaagtgctcatcattggaaaacgttcttcggggcgaaaactctcaaggatcttac
cgctgttgagatccagttcgatgtaacccactcgtgcacccaactgatcttcagcatcttttactttcaccagcgtttctgggtgagcaaaaaca
ggaaggcaaaatgccgcaaaaaagggaataagggcgacacggaaatgttgaatactcatactcttcctttttcaatattattgaagcatttatc
agggttattgtctcatgagcggatacatatttgaatgtatttagaaaaataaacaaataggggttccgcgcacatttccccgaaaagtgccacc
tgac.

In an aspect, a disclosed pBK744 plasmid can comprise the sequence set forth below or a sequence having 70%, 75%, 80%, 85%, 90%, 95%, or more identity to the sequence set forth

(SEQ ID NO: 24)
gtcgacggatcgggagatctcccgatcccctatggtgcactctcagtacaatctgctctgatgccgcatagttaagccagtatctgctccctg
cttgtgtgttggaggtcgctgagtagtgcgcgagcaaaatttaagctacaacaaggcaaggcttgaccgacaattgcatgaagaatctgctt
agggttaggcgttttgcgctgcttcgcgatgtacgggccagatatacgcgttgacattgattattgactagttattaatagtaatcaattacggg
gtcattagttcatagcccatatatggagttccgcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtacatcaatgggcgtggata
gcggtttgactcacggggatttccaagtctccaccccattgacgtcaatgggagtttgttttggcaccaaaatcaacgggactttccaaaatgt
cgtaacaactccgccccattgacgcaaatgggcggtaggcgtgtacggtgggaggtctatataagcagcgcgttttgcctgtactgggtct
ctctggttagaccagatctgagcctgggagctctctggctaactagggaacccactgcttaagcctcaataaagcttgccttgagtgcttcaa
gtagtgtgtgcccgtctgttgtgtgactctggtaactagagatccctcagacccttttagtcagtgtggaaaatctctagcagtggcgcccgaa
cagggacttgaaagcgaaagggaaaccagaggagctctctcgacgcaggactcggcttgctgaagcgcgcacggcaagaggcgagg
ggcggcgactggtgagtacgccaaaaattttgactagcggaggctagaaggagagagatgggtgcgagagcgtcagtattaagcgggg
gagaattagatcgcgatgggaaaaaattcggttaaggccagggggaaagaaaaaatataaattaaaacatatagtatgggcaagcaggga
gctagaacgattcgcagttaatcctggcctgttagaaacatcagaaggctgtagacaaatactgggacagctacaaccatcccttcagacag
gatcagaagaacttagatcattatataatacagtagcaaccctctattgtgtgcatcaaaggatagagataaaagacaccaaggaagctttag
acaagatagaggaagagcaaaacaaaagtaagaccaccgcacagcaagcggccgctgatcttcagacctggaggaggagatatgagg
gacaattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatctgttgca
actcacagtctggggcatcaagcagctccaggcaagaatcctggctgtggaaagatacctaaaggatcaacagctcctggggatttgggg
ttgctctggaaaactcatttgcaccactgctgtgccttggaatgctagttggagtaataaatctctggaacagatttggaatcacacgacctgg
atggagtgggacagagaaattaacaattacacaagcttaatacactccttaattgaagaatcgcaaaaccagcaagaaaagaatgaacaag
aattattggaattagataaatgggcaagtttgtggaattggtttaacataacaaattggctgtggtatataaaattattcataatgatagtaggag
gcttggtaggtttaagaatagtttttgctgtactttctatagtgaatagagttaggcagggatattcaccattatcgtttcagacccacctcccaac
cccgaggggacccgacaggcccgaaggaatagaagaagaaggtggagagagagacagagacagatccattcgattagtgaacggatc
ggcactgcgtgcgccaattctgcagacaaatggcagtattcatccacaattttaaaagaaaaggggggattggggggtacagtgcagggg
aaagaatagtagacataatagcaacagacatacaaactaaagaattacaaaaacaaattacaaaaattcaaaattttcgggtttattacaggg
acagcagagatccagtttggttaattaatggggggacgttaacggggcggaacggtaccgagggcctatttcccatgattccttcatatttgca
tatacgatacaaggctgttagagagataattagaattaatttgactgtaaacacaaagatattagtacaaaatacgtgacgtagaaagtaataattt
cttgggtagtttgcagttttaaaattatgttttaaaatggactatcatatgcttaccgtaacttgaaagtatttcgatttcttggctttatatatct
tgtggaaaggacgaaacaccgtttttcaagcggaaacgctagttttagagctagaaatagcaagttaaaataaggctagtccgttatcaactt
gaaaaagtggcaccgagtcggtgcttttttgaattcgctagctaggtcttgaaaggagtgggaattggctccggtgcccgtcagtgggcag
agcgcacatcgcccacagtccccgagaagttggggggaggggtcggcaattgatccggtgcctagagaaggtggcgcggggtaaact
gggaaagtgatgtcgtgtactggctccgcctttttcccgaggggggggagaaccgtatataagtgcagtagtcgccgtgaacgttctttttc
gcaacgggtttgccgccagaacacaggaccggtgccaccatggactataaggaccacgacggagactacaaggatcatgatattgattac
aaagacgatgacgataagatggccccaaagaagaagcggaaggtcggtatccacggagtcccagcagccgacaagaagtacagcatc
ggcctggccatcggcaccaactctgtgggctgggccgtgatcaccgacgagtacaaggtgcccagcaagaaattcaaggtgctgggcaa
caccgaccggcacagcatcaagaagaacctgatcggagccctgctgttcgacagcggcgaaacagccgaggccacccggctgaagag
aaccgccagaagaagatacaccagacggaagaaccggatctgctatctgcaagagatcttcagcaacgagatggccaaggtggacgac
agcttcttccacagactggaagagtccttcctggtggaagaggataagaagcacgagcggcaccccatcttcggcaacatcgtggacgag
gtggcctaccacgagaagtaccccaccatctaccacctgagaaagaaactggtggacagcaccgacaaggccgacctgcggctgatcta
tctggccctggcccacatgatcaagttccggggccacttcctgatcgagggcgacctgaaccccgacaacagcgacgtggacaagctgtt
catccagctggtgcagacctacaaccagctgttcgaggaaaaccccatcaacgccagcggcgtggacgccaaggccatcctgtctgcca
gactgagcaagagcagacggctggaaaatctgatcgcccagctgcccggcgagaagaagaatggcctgttcggcaacctgattgccctg
agcctgggcctgacccccaacttcaagagcaacttcgacctggccgaggatgccaaactgcagctgagcaaggacacctacgacgacg
acctggacaacctgctggcccagatcggcgaccagtacgccgacctgtttctggccgccaagaacctgtccgacgccatcctgctgagcg
acatcctgagagtgaacaccgagatcaccaaggcccccctgagcgcctctatgatcaagagatacgacgagcaccaccaggacctgacc
ctgctgaaagctctcgtgcggcagcagctgcctgagaagtacaaagagattttcttcgaccagagcaagaacggctacgccggctacattg
acggcggagccagccaggaagagttctacaagttcatcaagcccatcctggaaaagatggacggcaccgaggaactgctcgtgaagct
gaacagagaggacctgctgcggaagcagcggaccttcgacaacggcagcatcccccaccagatccacctgggagagctgcacgccatt
ctgcggcggcaggaagatttttacccattcctgaaggacaaccgggaaaagatcgagaagatcctgaccttccgcatcccctactacgtgg
gccctctggccaggggaaacagcagattcgcctggatgaccagaaagagcgaggaaaccatcaccccctggaacttcgaggaagtggt
ggacaagggcgcttccgcccagagcttcatcgagcggatgaccaacttcgataagaacctgcccaacgagaaggtgctgcccaagcac
agcctgctgtacgagtacttcaccgtgtataacgagctgaccaaagtgaaatacgtgaccgagggaatgagaaagcccgccttcctgagc
ggcgagcagaaaaaggccatcgtggacctgctgttcaagaccaaccggaaagtgaccgtgaagcagctgaaagaggactacttcaaga
aaatcgagtgcttcgactccgtggaaatctccggcgtggaagatcggttcaacgcctccctgggcacataccacgatctgctgaaaattatc
aaggacaaggacttcctggacaatgaggaaaacgaggacattctggaagatatcgtgctgaccctgacactgtttgaggacagagagatg
atcgaggaacggctgaaaacctatgcccacctgttcgacgacaaagtgatgaagcagctgaagcggcggagatacaccggctggggca
ggctgagccggaagctgatcaacggcatccgggacaagcagtccggcaagacaatcctggatttcctgaagtccgacggcttcgccaac
agaaacttcatgcagctgatccacgacgacagcctgacctttaaagaggacatccagaaagcccaggtgtccggccagggcgatagcct
gcacgagcacattgccaatctggccggcagccccgccattaagaagggcatcctgcagacagtgaaggtggtggacgagctcgtgaaa
gtgatgggccggcacaagcccgagaacatcgtgatcgaaatggccagagagaaccagaccacccagaagggacagaagaacagccg
cgagagaatgaagcggatcgaagagggcatcaaagagctgggcagccagatcctgaaagaacaccccgtggaaaacacccagctgca
gaacgagaagctgtacctgtactacctgcagaatggggggatatgtacgtggaccaggaactggacatcaaccggctgtccgactacga
tgtggacgctatcgtgcctcagagctttctgaaggacgactccatcgacaacaaggtgctgaccagaagcgacaagaaccggggcaaga
gcgacaacgtgccctccgaagaggtcgtgaagaagatgaagaactactggcggcagctgctgaacgccaagctgattacccagagaaa
gttcgacaatctgaccaaggccgagagaggcggcctgagcgaactggataaggccggcttcatcaagagacagctggtggaaacccgg
cagatcacaaagcacgtggcacagatcctggactcccggatgaacactaagtacgacgagaatgacaagctgatccgggaagtgaaagt
gatcaccctgaagtccaagctggtgtccgatttccggaaggatttccagttttacaaagtgcgcgagatcaacaactaccaccacgcccacg
acgcctacctgaacgccgtcgtgggaaccgccctgatcaaaaagtaccctaagctggaaagcgagttcgtgtacggcgactacaaggtgt
acgacgtgcggaagatgatcgccaagagcgagcaggaaatcggcaaggctaccgccaagtacttcttctacagcaacatcatgaacttttt
caagaccgagattaccctggccaacggcgagatccggaagcggcctctgatcgagacaaacggcgaaaccggggagatcgtgtggga
taagggccgggattttgccaccgtgcggaaagtgctgagcatgccccaagtgaatatcgtgaaaaagaccgaggtgcagacaggcggct
tcagcaaagagtctatcctgcccaagaggaacagcgataagctgatcgccagaaagaaggactgggaccctaagaagtacggcggcttc
gacagccccaccgtggcctattctgtgctggtggtggccaaagtggaaaagggcaagtccaagaaactgaagagtgtgaaagagctgct
ggggatcaccatcatggaaagaagcagcttcgagaagaatcccatcgactttctggaagccaagggctacaaagaagtgaaaaaggacc
tgatcatcaagctgcctaagtactccctgttcgagctggaaaacggccggaagagaatgctggcctctgccggcgaactgcagaagggaa
acgaactggccctgccctccaaatatgtgaacttcctgtacctggccagccactatgagaagctgaagggctcccccgaggataatgagca
gaaacagctgtttgtggaacagcacaagcactacctggacgagatcatcgagcagatcagcgagttctccaagagagtgatcctggccga
cgctaatctggacaaagtgctgtccgcctacaacaagcaccgggataagcccatcagagagcaggccgagaatatcatccacctgtttac
cctgaccaatctgggagcccctgccgccttcaagtactttgacaccaccatcgaccggaagaggtacaccagcaccaaagaggtgctgg
acgccaccctgatccaccagagcatcaccggcctgtacgagacacggatcgacctgtctcagctgggaggcgacaaaaggccggcgg
ccacgaaaaaggccggacaggccaaaaagaaaaagctcgagggcggaggcgggagcggatccccctcccggctccagatgttcttcg
ctaataaccacgaccaggaatttgaccctccaaaggtttacccacctgtcccagctgagaagaggaagcccatccgggtgctgtctctcttt
gatggaatcgctacagggctcctggtgctgaaggacttgggcattcaggtggaccgctacattgcctcggaggtgtgtgaggactccatca
cggtgggcatggtgcggcaccaggggaagatcatgtacgtcggggacgtccgcagcgtcacacagaagcatatccaggagtggggcc
cattcgatctggtgattgggggcagtccctgcaatgacctctccatcgtcaaccctgctcgcaagggcctctacgagggcactggccggct
cttctttgagttctaccgcctcctgcatgatgcgcggcccaaggagggagatgatcgccccttcttctggctctttgagaatgtggtggccatg
ggcgttagtgacaagagggacatctcgcgatttctcgagtccaaccctgtgatgattgatgccaaagaagtgtcagctgcacacagggccc
gctacttctggggtaaccttcccggtatgaacaggccgttggcatccactgtgaatgataagctggagctgcaggagtgtctggagcatgg
caggatagccaagttcagcaaagtgaggaccattactacgaggtcaaactccataaagcagggcaaagaccagcattttcctgtgttcatg
aatgagaaagaggacatcttatggtgcactgaaatggaaagggtatttggtttcccagtccactatactgacgtgtccaacatgagccgcttg
gcgaggcagagactgctgggccggtcatggagcgtgccagtcatccgccacctcttcgctccgctgaaggagtattttgcgtgtgtgtccg
gccggggccggcccggatccggcgcaacaaacttctctctgctgaaacaagccggagatgtcgaagagaatcctggaccgatggtgag
caagggcgaggagctgttcaccggggtggtgcccatcctggtcgagctggacggcgacgtaaacggccacaagttcagcgtgtccggc
gagggcgagggcgatgccacctacggcaagctgaccctgaagttcatctgcaccaccggcaagctgcccgtgccctggcccaccctcgt
gaccaccctgacctacggcgtgcagtgcttcagccgctaccccgaccacatgaagcagcacgacttcttcaagtccgccatgcccgaagg
ctacgtccaggagcgcaccatcttcttcaaggacgacggcaactacaagacccgcgccgaggtgaagttcgagggcgacaccctggtga
accgcatcgagctgaagggcatcgacttcaaggaggacggcaacatcctggggcacaagctggagtacaactacaacagccacaacgt
ctatatcatggccgacaagcagaagaacggcatcaaggtgaacttcaagatccgccacaacatcgaggacggcagcgtgcagctcgcc
gaccactaccagcagaacacccccatcggcgacggccccgtgctgctgcccgacaaccactacctgagcacccagtccgccctgagca
aagaccccaacgagaagcgcgatcacatggtcctgctggagttcgtgaccgccgccgggatcactctcggcatggacgagctgtacaag
taaagcggccgcgtcgacaatcaacctctggattacaaaatttgtgaaagattgactggtattcttaactatgttgctccttttacgctatgtgga
tacgctgctttaatgcctttgtatcatgctattgcttcccgtatggctttcattttctcctccttgtataaatcctggttgctgtctctttatgagg
agttgtggcccgttgtcaggcaacgtggcgtggtgtgcactgtgtttgctgacgcaacccccactggttggggcattgccaccacctgtcagctc
ctttccgggactttcgctttccccctccctattgccacggeggaactcatcgccgcctgccttgcccgctgctggacaggggctcggctgttg
ggcactgacaattccgtggtgttgtcggggaagctgacgtcctttccatggctgctcgcctgtgttgccacctggattctgcgcgggacgtc
cttctgctacgtcccttcggccctcaatccagcggaccttccttcccgcggcctgctgccggctctgcggcctcttccgcgtcttcgccttcgc
cctcagacgagtcggatctccctttgggccgcctccccgcctggaattcgagctcggtacctttaagaccaatgacttacaaggcagctgta
gatcttagccactttttaaaagaaaaggggggactggaagggctaattcactcccaacgaagacaagatctgctttttgcttgtactgggtctc
tctggttagaccagatctgagcctgggagctctctggctaactagggaacccactgcttaagcctcaataaagcttgccttgagtgcttcaag
tagtgtgtgcccgtctgttgtgtgactctggtaactagagatccctcagacccttttagtcagtgtggaaaatctctagcagtagtagttcatgtc
atcttattattcagtatttataacttgcaaagaaatgaatatcagagagtgagaggaacttgtttattgcagcttataatggttacaaataaagcaa
tagcatcacaaatttcacaaataaagcatttttttcactgcattctagttgtggtttgtccaaactcatcaatgtatcttatcatgtctggctctag
ctatcccgcccctaactccgcccatcccgcccctaactccgcccagttccgcccattctccgccccatggctgactaattttttttatttatgcaga
ggccgaggccgcctcggcctctgagctattccagaagtagtgaggaggcttttttggaggcctagggacgtacccaattcgccctatagtga
gtcgtattacgcgcgctcactggccgtcgttttacaacgtcgtgactgggaaaaccctggcgttacccaacttaatcgccttgcagcacatcc
ccctttcgccagctggcgtaatagcgaagaggcccgcaccgatcgcccttcccaacagttgcgcagcctgaatggcgaatgggacgcgc
cctgtagcggcgcattaagcgcggcgggtgtggtggttacgcgcagcgtgaccgctacacttgccagcgccctagcgcccgctcctttcg
ctttcttcccttcctttctcgccacgttcgccggctttccccgtcaagctctaaatcgggggctccctttagggttccgatttagtgctttacggca
cctcgaccccaaaaaacttgattagggtgatggttcacgtagtgggccatcgccctgatagacggtttttcgccctttgacgttggagtccacgtt
ctttaatagtggactcttgttccaaactggaacaacactcaaccctatctcggtctattcttttgatttataagggattttgccgatttcggccta
ttggttaaaaaatgagctgatttaacaaaaatttaacgcgaattttaacaaaatattaacgcttacaatttaggtgccggccatgaccgagatcg
gcgagcagccgtggggggggagttcgccctgcgcgacccggccggcaactgcgtgcacttcgtggccgaggagcaggactgacac
gtgctacgagatttcgattccaccgccgccttctatgaaaggttgggcttcggaatcgttttccgggacgccggctggatgatcctccagcg
cggggatctcatgctggagttcttcgcccaccccaacttgtttattgcagcttataatggttacaaataaagcaatagcatcacaaatttcacaaat
aaagcatttttttcactgcattctagttgtggtttgtccaaactcatcaatgtatcttatcatgtctgtataccgtcgacctctagctagagcttgg
cgtaatcatggtcatagctgtttcctgtgtgaaattgttatccgctcacaattccacacaacatacgagccggaagcataaagtgtaaagcctg
gggtgcctaatgagtgagctaactcacattaattgcgttgcgctcactgcccgctttccagtcgggaaacctgtcgtgccagctgcattaatg
aatcggccaacgcgcggggagaggcggtttgcgtattgggcgctcttccgcttcctcgctcactgactcgctgcgctcggtcgttcggctg
cggcgagcggtatcagctcactcaaaggcggtaatacggttatccacagaatcaggggataacgcaggaaagaacatgtgagcaaaag
gccagcaaaaggccaggaaccgtaaaaaggccgcgttgctggcgtttttccataggctccgcccccctgacgagcatcacaaaaatcga
cgctcaagtcagaggtggcgaaacccgacaggactataaagataccaggcgtttccccctggaagctccctcgtgcgctctcctgttccga
ccctgccgcttaccggatacctgtccgcctttctcccttcgggaagcgtggcgctttctcatagctcacgctgtaggtatctcagttcggtgta
ggtcgttcgctccaagctgggctgtgtgcacgaaccccccgttcagcccgaccgctgcgccttatccggtaactatcgtcttgagtccaacc
cggtaagacacgacttatcgccactggcagcagccactggtaacaggattagcagagcgaggtatgtaggcggtgctacagagttcttga
agtggtggcctaactacggctacactagaagaacagtatttggtatctgcgctctgctgaagccagttaccttcggaaaaagagttggtagct
cttgatccggcaaacaaaccaccgctggtagcggtggtttttttgtttgcaagcagcagattacgcgcagaaaaaaaggatctcaagaagat
cctttgatcttttctacggggtctgacgctcagtggaacgaaaactcacgttaagggattttggtcatgagattatcaaaaaggatcttcaccta
gatccttttaaattaaaaatgaagttttaaatcaatctaaagtatatatgagtaaacttggtctgacagttaccaatgcttaatcagtgaggcacct
atctcagcgatctgtctatttcgttcatccatagttgcctgactccccgtcgtgtagataactacgatacgggagggcttaccatctggcccca
gtgctgcaatgataccgcgagacccacgctcaccggctccagatttatcagcaataaaccagccagccggaagggccgagcgcagaag
tggtcctgcaactttatccgcctccatccagtctattaattgttgccgggaagctagagtaagtagttcgccagttaatagtttgcgcaacgttgt
tgccattgctacaggcatcgtggtgtcacgctcgtcgtttggtatggcttcattcagctccggttcccaacgatcaaggcgagttacatgatcc
cccatgttgtgcaaaaaagcggttagctccttcggtcctccgatcgttgtcagaagtaagttggccgcagtgttatcactcatggttatggcag
cactgcataattctcttactgtcatgccatccgtaagatgcttttctgtgactggtgagtactcaaccaagtcattctgagaatagtgtatgcggc
gaccgagttgctcttgcccggcgtcaatacgggataataccgcgccacatagcagaactttaaaagtgctcatcattggaaaacgttcttcg
gggcgaaaactctcaaggatcttaccgctgttgagatccagttcgatgtaacccactcgtgcacccaactgatcttcagcatcttttactttcac
cagcgtttctgggtgagcaaaaacaggaaggcaaaatgccgcaaaaaagggaataagggcgacacggaaatgttgaatactcatactctt
cctttttcaatattattgaagcatttatcagggttattgtctcatgagcggatacatatttgaatgtatttagaaaaataaacaaataggggttcc
gcgcacatttccccgaaaagtgccacctgac.

In an aspect, a disclosed pBK1026 plasmid can comprise the sequence set forth below or a sequence having 70%, 75%, 80%, 85%, 90%, 95%, or more identity to the sequence set forth

(SEQ ID NO: 29)
gtcgacggatcgggagatctcccgatcccctatggtgcactctcagtacaatctgctctgatgccgcatagttaagccagtatctgctccctg
cttgtgtgttggaggtcgctgagtagtgcgcgagcaaaatttaagctacaacaaggcaaggcttgaccgacaattgcatgaagaatctgctt
agggttaggcgttttgcgctgcttcgcgatgtacgggccagatatacgcgttgacattgattattgactagttattaatagtaatcaattacggg
gtcattagttcatagcccatatatggagttccgcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtacatcaatgggcgtggata
gcggtttgactcacggggatttccaagtctccaccccattgacgtcaatgggagtttgttttggcaccaaaatcaacgggactttccaaaatgt
cgtaacaactccgccccattgacgcaaatgggcggtaggcgtgtacggtgggaggtctatataagcagcgcgttttgcctgtactgggtct
ctctggttagaccagatctgagcctgggagctctctggctaactagggaacccactgcttaagcctcaataaagcttgccttgagtgcttcaa
gtagtgtgtgcccgtctgttgtgtgactctggtaactagagatccctcagacccttttagtcagtgtggaaaatctctagcagtggcgcccgaa
cagggacttgaaagcgaaagggaaaccagaggagctctctcgacgcaggactcggcttgctgaagcgcgcacggcaagaggcgagg
ggcggcgactggtgagtacgccaaaaattttgactagcggaggctagaaggagagagatgggtgcgagagcgtcagtattaagcgggg
gagaattagatcgcgatgggaaaaaattcggttaaggccagggggaaagaaaaaatataaattaaaacatatagtatgggcaagcaggga
gctagaacgattcgcagttaatcctggcctgttagaaacatcagaaggctgtagacaaatactgggacagctacaaccatcccttcagacag
gatcagaagaacttagatcattatataatacagtagcaaccctctattgtgtgcatcaaaggatagagataaaagacaccaaggaagctttag
acaagatagaggaagagcaaaacaaaagtaagaccaccgcacagcaagcggccgctgatcttcagacctggaggaggagatatgagg
gacaattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatctgttgca
actcacagtctggggcatcaagcagctccaggcaagaatcctggctgtggaaagatacctaaaggatcaacagctcctggggatttgggg
ttgctctggaaaactcatttgcaccactgctgtgccttggaatgctagttggagtaataaatctctggaacagatttggaatcacacgacctgg
atggagtgggacagagaaattaacaattacacaagcttaatacactccttaattgaagaatcgcaaaaccagcaagaaaagaatgaacaag
aattattggaattagataaatgggcaagtttgtggaattggtttaacataacaaattggctgtggtatataaaattattcataatgatagtaggag
gcttggtaggtttaagaatagtttttgctgtactttctatagtgaatagagttaggcagggatattcaccattategtttcagacccacctcccaac
cccgaggggacccgacaggcccgaaggaatagaagaagaaggtggagagagagacagagacagatccattcgattagtgaacggatc
ggcactgcgtgcgccaattctgcagacaaatggcagtattcatccacaattttaaaagaaaaggggggattggggggtacagtgcagggg
aaagaatagtagacataatagcaacagacatacaaactaaagaattacaaaaacaaattacaaaaattcaaaattttcgggtttattacaggg
acagcagagatccagtttggttaattaatggggggacgttaacggggcggaacggtaccgagggcctatttcccatgattccttcatatttg
catatacgatacaaggctgttagagagataattagaattaatttgactgtaaacacaaagatattagtacaaaatacgtgacgtagaaagtaataat
ttcttgggtagtttgcagttttaaaattatgttttaaaatggactatcatatgcttaccgtaacttgaaagtatttcgatttcttggctttatatat
cttgtggaaaggacgaaacaccggacagggggagccctataatgttttagagctagaaatagcaagttaaaataaggctagtccgttatcaac
ttgaaaaagtggcaccgagtcggtgcttttttgaattcgctagctaggtcttgaaaggagtgggaattggctccggtgcccgtcagtgggca
gagcgcacatcgcccacagtccccgagaagttggggggaggggtcggcaattgatccggtgcctagagaaggtggcgcggggtaaac
tgggaaagtgatgtcgtgtactggctccgcctttttcccgaggggggggagaaccgtatataagtgcagtagtcgccgtgaacgttctttttc
gcaacgggtttgccgccagaacacaggaccggtgccaccatggactataaggaccacgacggagactacaaggatcatgatattgattac
aaagacgatgacgataagatggccccaaagaagaagcggaaggtcggtatccacggagtcccagcagccgacaagaagtacagcatc
ggcctggccatcggcaccaactctgtgggctgggccgtgatcaccgacgagtacaaggtgcccagcaagaaattcaaggtgctgggcaa
caccgaccggcacagcatcaagaagaacctgatcggagccctgctgttcgacagcggcgaaacagccgaggccacccggctgaagag
aaccgccagaagaagatacaccagacggaagaaccggatctgctatctgcaagagatcttcagcaacgagatggccaaggtggacgac
agcttcttccacagactggaagagtccttcctggtggaagaggataagaagcacgagcggcaccccatcttcggcaacatcgtggacgag
gtggcctaccacgagaagtaccccaccattaccacctgagaaagaaactggtggacagcaccgacaaggccgacctgcggctgatcta
tctggccctggcccacatgatcaagttccggggccacttcctgatcgagggcgacctgaaccccgacaacagcgacgtggacaagctgtt
catccagctggtgcagacctacaaccagctgttcgaggaaaaccccatcaacgccagcggcgtggacgccaaggccatcctgtctgcca
gactgagcaagagcagacggctggaaaatctgatcgcccagctgcccggcgagaagaagaatggcctgttcggcaacctgattgccctg
agcctgggcctgacccccaacttcaagagcaacttcgacctggccgaggatgccaaactgcagctgagcaaggacacctacgacgacg
acctggacaacctgctggcccagatcggcgaccagtacgccgacctgtttctggccgccaagaacctgtccgacgccatcctgctgagcg
acatcctgagagtgaacaccgagatcaccaaggcccccctgagcgcctctatgatcaagagatacgacgagcaccaccaggacctgacc
ctgctgaaagctctcgtgcggcagcagctgcctgagaagtacaaagagattttcttcgaccagagcaagaacggctacgccggctacattg
acggcggagccagccaggaagagttctacaagttcatcaagcccatcctggaaaagatggacggcaccgaggaactgctcgtgaagct
gaacagagaggacctgctgcggaagcagcggaccttcgacaacggcagcatcccccaccagatccacctgggagagctgcacgccatt
ctgcggcggcaggaagatttttacccattcctgaaggacaaccgggaaaagatcgagaagatcctgaccttccgcatcccctactacgtgg
gccctctggccaggggaaacagcagattcgcctggatgaccagaaagagcgaggaaaccatcaccccctggaacttcgaggaagtggt
ggacaagggcgcttccgcccagagcttcatcgagcggatgaccaacttcgataagaacctgcccaacgagaaggtgctgcccaagcac
agcctgctgtacgagtacttcaccgtgtataacgagctgaccaaagtgaaatacgtgaccgagggaatgagaaagcccgccttcctgagc
ggcgagcagaaaaaggccatcgtggacctgctgttcaagaccaaccggaaagtgaccgtgaagcagctgaaagaggactacttcaaga
aaatcgagtgcttcgactccgtggaaatctccggcgtggaagatcggttcaacgcctccctgggcacataccacgatctgctgaaaattatc
aaggacaaggacttcctggacaatgaggaaaacgaggacattctggaagatatcgtgctgaccctgacactgtttgaggacagagagatg
atcgaggaacggctgaaaacctatgcccacctgttcgacgacaaagtgatgaagcagctgaagcggcggagatacaccggctggggca
ggctgagccggaagctgatcaacggcatccgggacaagcagtccggcaagacaatcctggatttcctgaagtccgacggcttcgccaac
agaaacttcatgcagctgatccacgacgacagcctgacctttaaagaggacatccagaaagcccaggtgtccggccagggcgatagcct
gcacgagcacattgccaatctggccggcagccccgccattaagaagggcatcctgcagacagtgaaggtggtggacgagctcgtgaaa
gtgatgggccggcacaagcccgagaacatcgtgatcgaaatggccagagagaaccagaccacccagaagggacagaagaacagccg
cgagagaatgaagcggatcgaagagggcatcaaagagctgggcagccagatcctgaaagaacaccccgtggaaaacacccagctgca
gaacgagaagctgtacctgtactacctgcagaatggggggatatgtacgtggaccaggaactggacatcaaccggctgtccgactacga
tgtggacgctatcgtgcctcagagctttctgaaggacgactccatcgacaacaaggtgctgaccagaagcgacaagaaccggggcaaga
gcgacaacgtgccctccgaagaggtcgtgaagaagatgaagaactactggcggcagctgctgaacgccaagctgattacccagagaaa
gttcgacaatctgaccaaggccgagagaggcggcctgagcgaactggataaggccggcttcatcaagagacagctggtggaaacccgg
cagatcacaaagcacgtggcacagatcctggactcccggatgaacactaagtacgacgagaatgacaagctgatccgggaagtgaaagt
gatcaccctgaagtccaagctggtgtccgatttccggaaggatttccagttttacaaagtgcgcgagatcaacaactaccaccacgcccacg
acgcctacctgaacgccgtcgtgggaaccgccctgatcaaaaagtaccctaagctggaaagcgagttcgtgtacggcgactacaaggtgt
acgacgtgcggaagatgatcgccaagagcgagcaggaaatcggcaaggctaccgccaagtacttcttctacagcaacatcatgaacttttt
caagaccgagattaccctggccaacggcgagatccggaagcggcctctgatcgagacaaacggcgaaaccggggagatcgtgtggga
taagggccgggattttgccaccgtgcggaaagtgctgagcatgccccaagtgaatatcgtgaaaaagaccgaggtgcagacaggcggct
tcagcaaagagtctatcctgcccaagaggaacagcgataagctgatcgccagaaagaaggactgggaccctaagaagtacggcggcttc
gacagccccaccgtggcctattctgtgctggtggtggccaaagtggaaaagggcaagtccaagaaactgaagagtgtgaaagagctgct
ggggatcaccatcatggaaagaagcagcttcgagaagaatcccatcgactttctggaagccaagggctacaaagaagtgaaaaaggacc
tgatcatcaagctgcctaagtactccctgttcgagctggaaaacggccggaagagaatgctggcctctgccggcgaactgcagaagggaa
acgaactggccctgccctccaaatatgtgaacttcctgtacctggccagccactatgagaagctgaagggctcccccgaggataatgagca
gaaacagctgtttgtggaacagcacaagcactacctggacgagatcatcgagcagatcagcgagttctccaagagagtgatcctggccga
cgctaatctggacaaagtgctgtccgcctacaacaagcaccgggataagcccatcagagagcaggccgagaatatcatccacctgtttac
cctgaccaatctgggagcccctgccgccttcaagtactttgacaccaccatcgaccggaagaggtacaccagcaccaaagaggtgctgg
acgccaccctgatccaccagagcatcaccggcctgtacgagacacggatcgacctgtctcagctgggaggcgacaaaaggccggcgg
ccacgaaaaaggccggacaggccaaaaagaaaaagctcgagggcggaggcgggagcggatccccctcccggctccagatgttcttcg
ctaataaccacgaccaggaatttgaccctccaaaggtttacccacctgtcccagctgagaagaggaagcccatccgggtgctgtctctcttt
gatggaatcgctacagggctcctggtgctgaaggacttgggcattcaggtggaccgctacattgcctcggaggtgtgtgaggactccatca
cggtgggcatggtgcggcaccaggggaagatcatgtacgtcggggacgtccgcagcgtcacacagaagcatatccaggagtggggcc
cattcgatctggtgattgggggcagtccctgcaatgacctctccatcgtcaaccctgctcgcaagggcctctacgagggcactggccggct
cttctttgagttctaccgcctcctgcatgatgcgcggcccaaggagggagatgatcgccccttcttctggctctttgagaatgtggtggccatg
ggcgttagtgacaagagggacatctcgcgatttctcgagtccaaccctgtgatgattgatgccaaagaagtgtcagctgcacacagggccc
gctacttctggggtaaccttcccggtatgaacaggccgttggcatccactgtgaatgataagctggagctgcaggagtgtctggagcatgg
caggatagccaagttcagcaaagtgaggaccattactacgaggtcaaactccataaagcagggcaaagaccagcattttcctgtgttcatg
aatgagaaagaggacatcttatggtgcactgaaatggaaagggtatttggtttcccagtccactatactgacgtgtccaacatgagccgcttg
gcgaggcagagactgctgggccggtcatggagcgtgccagtcatccgccacctcttcgctccgctgaaggagtattttgcgtgtgtgtccg
gccggcccggatccggcgcaacaaacttctctctgctgaaacaagccggagatgtcgaagagaatcctggaccgaccgagtacaagcc
cacggtgcgcctcgccacccgcgacgacgtccccagggccgtacgcaccctcgccgccgegttcgccgactaccccgccacgcgcca
caccgtcgatccggaccgccacatcgagcgggtcaccgagctgcaagaactcttcctcacgcgcgtcgggctcgacatcggcaaggtgt
gggtcgcggacgacggcgccgcggtggcggtctggaccacgccggagagcgtcgaagcgggggcggtgttcgccgagatcggccc
gcgcatggccgagttgagcggttcccggctggccgcgcagcaacagatggaaggcctcctggcgccgcaccggcccaaggagcccg
cgtggttcctggccaccgtcggagtctcgcccgaccaccagggcaagggtctgggcagcgccgtcgtgctccccggagtggaggcgg
ccgagcgcgccggggtgcccgccttcctggagacctccgcgccccgcaacctccccttctacgageggcteggcttcaccgtcaccgcc
gacgtcgaggtgcccgaaggaccgcgcacctggtgcatgacccgcaagcccggtgcctgaacgcgttaagtcgacaatcaacctctggattaca
aaatttgtgaaagattgactggtattcttaactatgttgctccttttacgctatgtggatacgctgctttaatgcctttgtatcatgctattgc
ttcccgtatggctttcattttctcctccttgtataaatcctggttgctgtctctttatgaggagttgtggcccgttgtcaggcaacgtggcgtg
gtgtgcactgtgtttgctgacgcaacccccactggttggggcattgccaccacctgtcagctcctttccgggactttcgctttccccctcccta
ttgccacggcggaactcatcgccgcctgccttgcccgctgctggacaggggctcggctgttgggcactgacaattccgtggtgttgtcggggaaa
tcatcgtcctttccttggctgctcgcctgtgttgccacctggattctgcgcgggacgtccttctgctacgtcccttcggccctcaatccagcgg
accttccttcccgcggcctgctgccggctctgcggcctcttccgcgtcttcgccttcgccctcagacgagtcggatctccctttgggccgcct
ccccgcgtcgactttaagaccaatgacttacaaggcagctgtagatcttagccactttttaaaagaaaaggggggactggaagggctaattc
actcccaacgaagacaagatctgctttttgcttgtactgggtctctctggttagaccagatctgagcctgggagctctctggctaactagggaa
cccactgcttaagcctcaataaagcttgccttgagtgcttcaagtagtgtgtgcccgtctgttgtgtgactctggtaactagagatccctcagac
ccttttagtcagtgtggaaaatctctagcagggcccgtttaaacccgctgatcagcctcgactgtgccttctagttgccagccatctgttgtttg
cccctcccccgtgccttccttgaccctggaaggtgccactcccactgtcctttcctaataaaatgaggaaattgcatcgcattgtctgagtagg
tgtcattctattctggggggtggggggggcaggacagcaagggggaggattgggaagacaatagcaggcatgctggggatgcggtgg
gctctatggcttctgaggcggaaagaaccagctggggctctagggggtatccccacgcgccctgtagcggcgcattaagcgcggcgggt
gtggtggttacgcgcagcgtgaccgctacacttgccagcgccctagcgcccgctcctttcgctttcttcccttcctttctcgccacgttcgccg
gctttccccgtcaagctctaaatcgggggctccctttagggttccgatttagtgctttacggcacctcgaccccaaaaaacttgattagggtga
tggttcacgtagtgggccatcgccctgatagacggtttttcgccctttgacgttggagtccacgttctttaatagtggactcttgttccaaactgg
aacaacactcaaccctatctcggtctattcttttgatttataagggattttgccgatttcggcctattggttaaaaaatgagctgatttaacaaaa
atttaacgcgaattaattctgtggaatgtgtgtcagttagggtgtggaaagtccccaggctccccagcaggcagaagtatgcaaagcatgcatc
tcaattagtcagcaaccaggtgtggaaagtccccaggctccccagcaggcagaagtatgcaaagcatgcatctcaattagtcagcaaccat
agtcccgcccctaactccgcccatcccgcccctaactccgcccagttccgcccattctccgccccatggctgactaattttttttatttatgcag
aggccgaggccgcctctgcctctgagctattccagaagtagtgaggaggcttttttggaggcctaggcttttgcaaaaagctcccgggagc
ttgtatatccattttcggatctgatcagcacgtgttgacaattaatcatcggcatagtatatcggcatagtataatacgacaaggtgaggaacta
aaccatggccaagttgaccagtgccgttccggtgctcaccgcgcgcgacgtcgccggagcggtcgagttctggaccgaccggctcgggt
tctcccgggacttcgtggaggacgacttcgccggtgtggtccgggacgacgtgaccctgttcatcagcgcggtccaggaccaggtggtg
ccggacaacaccctggcctgggtgtgggtgcgcggcctggacgagctgtacgccgagtggtcggaggtcgtgtccacgaacttccggg
acgcctccgggccggccatgaccgagatcggcgagcagccgtggggggggagttcgccctgcgcgacccggccggcaactgcgtg
cacttcgtggccgaggagcaggactgacacgtgctacgagatttcgattccaccgccgccttctatgaaaggttgggcttcggaatcgttttc
cgggacgccggctggatgatcctccagcgcggggatctcatgctggagttcttcgcccaccccaacttgtttattgcagcttataatggttacaaa
taaagcaatagcatcacaaatttcacaaataaagcatttttttcactgcattctagttgtggtttgtccaaactcatcaatgtatcttatcatgtc
tgtataccgtcgacctctagctagagcttggcgtaatcatggtcatagctgtttcctgtgtgaaattgttatccgctcacaattccacacaacata
cgagccggaagcataaagtgtaaagcctggggtgcctaatgagtgagctaactcacattaattgcgttgcgctcactgcccgctttccagtc
gggaaacctgtcgtgccagctgcattaatgaatcggccaacgcgcggggagaggcggtttgcgtattgggcgctcttccgcttcctcgctc
actgactcgctgcgctcggtcgttcggctgcggcgagcggtatcagctcactcaaaggcggtaatacggttatccacagaatcaggggata
acgcaggaaagaacatgtgagcaaaaggccagcaaaaggccaggaaccgtaaaaaggccgcgttgctggcgtttttccataggctccgc
ccccctgacgagcatcacaaaaatcgacgctcaagtcagaggtggcgaaacccgacaggactataaagataccaggcgtttccccctgg
aagctccctcgtgcgctctcctgttccgaccctgccgcttaccggatacctgtccgcctttctcccttcgggaagcgtggcgctttctcatagc
tcacgctgtaggtatctcagttcggtgtaggtcgttcgctccaagctgggctgtgtgcacgaaccccccgttcagcccgaccgctgcgcctt
atccggtaactatcgtcttgagtccaacccggtaagacacgacttatcgccactggcagcagccactggtaacaggattagcagagcgag
gtatgtaggcggtgctacagagttcttgaagtggtggcctaactacggctacactagaagaacagtatttggtatctgcgctctgctgaagcc
agttaccttcggaaaaagagttggtagctcttgatccggcaaacaaaccaccgctggtagcggtggtttttttgtttgcaagcagcagattac
gcgcagaaaaaaaggatctcaagaagatcctttgatcttttctacggggtctgacgctcagtggaacgaaaactcacgttaagggattttggt
catgagattatcaaaaaggatcttcacctagatccttttaaattaaaaatgaagttttaaatcaatctaaagtatatatgagtaaacttggtctgac
agttaccaatgcttaatcagtgaggcacctatctcagcgatctgtctatttcgttcatccatagttgcctgactccccgtcgtgtagataactacg
atacgggagggcttaccatctggccccagtgctgcaatgataccgcgagacccacgctcaccggctccagatttatcagcaataaaccag
ccagccggaagggccgagcgcagaagtggtcctgcaactttatccgcctccatccagtctattaattgttgccgggaagctagagtaagta
gttcgccagttaatagtttgcgcaacgttgttgccattgctacaggcatcgtggtgtcacgctcgtcgtttggtatggcttcattcagctccggtt
cccaacgatcaaggcgagttacatgatcccccatgttgtgcaaaaaagcggttagctccttcggtcctccgatcgttgtcagaagtaagttgg
ccgcagtgttatcactcatggttatggcagcactgcataattctcttactgtcatgccatccgtaagatgcttttctgtgactggtgagtactcaa
ccaagtcattctgagaatagtgtatgcggcgaccgagttgctcttgcccggcgtcaatacgggataataccgcgccacatagcagaacttta
aaagtgctcatcattggaaaacgttcttcggggcgaaaactctcaaggatcttaccgctgttgagatccagttcgatgtaacccactcgtgca
cccaactgatcttcagcatcttttactttcaccagcgtttctgggtgagcaaaaacaggaaggcaaaatgccgcaaaaaagggaataaggg
cgacacggaaatgttgaatactcatactcttcctttttcaatattattgaagcatttatcagggttattgtctcatgagcggatacatatttgaat
gtatttagaaaaataaacaaataggggttccgcgcacatttccccgaaaagtgccacctgac.

In an aspect, a disclosed pBK1027 plasmid can comprise the sequence set forth below or a sequence having 70%, 75%, 80%, 85%, 90%, 95%, or more identity to the sequence set forth

(SEQ ID NO: 30)
gtcgacggatcgggagatctcccgatcccctatggtgcactctcagtacaatctgctctgatgccgcatagttaagccagtatctgctccctg
cttgtgtgttggaggtcgctgagtagtgcgcgagcaaaatttaagctacaacaaggcaaggcttgaccgacaattgcatgaagaatctgctt
agggttaggcgttttgcgctgcttcgcgatgtacgggccagatatacgcgttgacattgattattgactagttattaatagtaatcaattacggg
gtcattagttcatagcccatatatggagttccgcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtacatcaatgggcgtggata
gcggtttgactcacggggatttccaagtctccaccccattgacgtcaatgggagtttgttttggcaccaaaatcaacgggactttccaaaatgt
cgtaacaactccgccccattgacgcaaatgggcggtaggcgtgtacggtgggaggtctatataagcagcgcgttttgcctgtactgggtct
ctctggttagaccagatctgagcctgggagctctctggctaactagggaacccactgcttaagcctcaataaagcttgccttgagtgcttcaa
gtagtgtgtgcccgtctgttgtgtgactctggtaactagagatccctcagacccttttagtcagtgtggaaaatctctagcagtggcgcccgaa
cagggacttgaaagcgaaagggaaaccagaggagctctctcgacgcaggactcggcttgctgaagcgcgcacggcaagaggcgagg
ggcggcgactggtgagtacgccaaaaattttgactagcggaggctagaaggagagagatgggtgcgagagcgtcagtattaagcgggg
gagaattagatcgcgatgggaaaaaattcggttaaggccagggggaaagaaaaaatataaattaaaacatatagtatgggcaagcaggga
gctagaacgattcgcagttaatcctggcctgttagaaacatcagaaggctgtagacaaatactgggacagctacaaccatcccttcagacag
gatcagaagaacttagatcattatataatacagtagcaaccctctattgtgtgcatcaaaggatagagataaaagacaccaaggaagctttag
acaagatagaggaagagcaaaacaaaagtaagaccaccgcacagcaagcggccgctgatcttcagacctggaggaggagatatgagg
gacaattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatctgttgca
actcacagtctggggcatcaagcagctccaggcaagaatcctggctgtggaaagatacctaaaggatcaacagctcctggggatttgggg
ttgctctggaaaactcatttgcaccactgctgtgccttggaatgctagttggagtaataaatctctggaacagatttggaatcacacgacctgg
atggagtgggacagagaaattaacaattacacaagcttaatacactccttaattgaagaatcgcaaaaccagcaagaaaagaatgaacaag
aattattggaattagataaatgggcaagtttgtggaattggtttaacataacaaattggctgtggtatataaaattattcataatgatagtaggag
gcttggtaggtttaagaatagtttttgctgtactttctatagtgaatagagttaggcagggatattcaccattatcgtttcagacccacctcccaac
cccgaggggacccgacaggcccgaaggaatagaagaagaaggtggagagagagacagagacagatccattcgattagtgaacggatc
ggcactgcgtgcgccaattctgcagacaaatggcagtattcatccacaattttaaaagaaaaggggggattggggggtacagtgcagggg
aaagaatagtagacataatagcaacagacatacaaactaaagaattacaaaaacaaattacaaaaattcaaaattttcgggtttattacaggg
acagcagagatccagtttggttaattaatggggggacgttaacggggggaacggtaccgagggcctatttcccatgattccttcatatttg
catatacgatacaaggctgttagagagataattagaattaatttgactgtaaacacaaagatattagtacaaaatacgtgacgtagaaagtaataat
ttcttgggtagtttgcagttttaaaattatgttttaaaatggactatcatatgcttaccgtaacttgaaagtatttcgatttcttggctttatatat
cttgtggaaaggacgaaacaccgtcaggagagctactcggggtgttttagagctagaaatagcaagttaaaataaggctagtccgttatcaact
tgaaaaagtggcaccgagtcggtgcttttttgaattcgctagctaggtcttgaaaggagtgggaattggctccggtgcccgtcagtgggcag
agcgcacatcgcccacagtccccgagaagttggggggaggggtcggcaattgatccggtgcctagagaaggtggcgcggggtaaact
gggaaagtgatgtcgtgtactggctccgcctttttcccgaggggggggagaaccgtatataagtgcagtagtcgccgtgaacgttctttttc
gcaacgggtttgccgccagaacacaggaccggtgccaccatggactataaggaccacgacggagactacaaggatcatgatattgattac
aaagacgatgacgataagatggccccaaagaagaagcggaaggtcggtatccacggagtcccagcagccgacaagaagtacagcatc
ggcctggccatcggcaccaactctgtgggctgggccgtgatcaccgacgagtacaaggtgcccagcaagaaattcaaggtgctgggcaa
caccgaccggcacagcatcaagaagaacctgatcggagccctgctgttcgacagcggcgaaacagccgaggccacccggctgaagag
aaccgccagaagaagatacaccagacggaagaaccggatctgctatctgcaagagatcttcagcaacgagatggccaaggtggacgac
agcttcttccacagactggaagagtccttcctggtggaagaggataagaagcacgagcggcaccccatcttcggcaacatcgtggacgag
gtggcctaccacgagaagtaccccaccatctaccacctgagaaagaaactggtggacagcaccgacaaggccgacctgcggctgatcta
tctggccctggcccacatgatcaagttccggggccacttcctgatcgagggcgacctgaaccccgacaacagcgacgtggacaagctgtt
catccagctggtgcagacctacaaccagctgttcgaggaaaaccccatcaacgccagcggcgtggacgccaaggccatcctgtctgcca
gactgagcaagagcagacggctggaaaatctgatcgcccagctgcccggcgagaagaagaatggcctgttcggcaacctgattgccctg
agcctgggcctgacccccaacttcaagagcaacttcgacctggccgaggatgccaaactgcagctgagcaaggacacctacgacgacg
acctggacaacctgctggcccagatcggcgaccagtacgccgacctgtttctggccgccaagaacctgtccgacgccatcctgctgagcg
acatcctgagagtgaacaccgagatcaccaaggcccccctgagcgcctctatgatcaagagatacgacgagcaccaccaggacctgacc
ctgctgaaagctctcgtgcggcagcagctgcctgagaagtacaaagagattttcttcgaccagagcaagaacggctacgccggctacattg
acggcggagccagccaggaagagttctacaagttcatcaagcccatcctggaaaagatggacggcaccgaggaactgctcgtgaagct
gaacagagaggacctgctgcggaagcagcggaccttcgacaacggcagcatcccccaccagatccacctgggagagctgcacgccatt
ctgcggcggcaggaagatttttacccattcctgaaggacaaccgggaaaagatcgagaagatcctgaccttccgcatcccctactacgtgg
gccctctggccaggggaaacagcagattcgcctggatgaccagaaagagcgaggaaaccatcaccccctggaacttcgaggaagtggt
ggacaagggcgcttccgcccagagcttcatcgagcggatgaccaacttcgataagaacctgcccaacgagaaggtgctgcccaagcac
agcctgctgtacgagtacttcaccgtgtataacgagctgaccaaagtgaaatacgtgaccgagggaatgagaaagcccgccttcctgagc
ggcgagcagaaaaaggccatcgtggacctgctgttcaagaccaaccggaaagtgaccgtgaagcagctgaaagaggactacttcaaga
aaatcgagtgcttcgactccgtggaaatctccggcgtggaagatcggttcaacgcctccctgggcacataccacgatctgctgaaaattatc
aaggacaaggacttcctggacaatgaggaaaacgaggacattctggaagatatcgtgctgaccctgacactgtttgaggacagagagatg
atcgaggaacggctgaaaacctatgcccacctgttcgacgacaaagtgatgaagcagctgaagcggcggagatacaccggctggggca
ggctgagccggaagctgatcaacggcatccgggacaagcagtccggcaagacaatcctggatttcctgaagtccgacggcttcgccaac
agaaacttcatgcagctgatccacgacgacagcctgacctttaaagaggacatccagaaagcccaggtgtccggccagggcgatagcct
gcacgagcacattgccaatctggccggcagccccgccattaagaagggcatcctgcagacagtgaaggtggtggacgagctcgtgaaa
gtgatgggccggcacaagcccgagaacatcgtgatcgaaatggccagagagaaccagaccacccagaagggacagaagaacagccg
cgagagaatgaagcggatcgaagagggcatcaaagagctgggcagccagatcctgaaagaacaccccgtggaaaacacccagctgca
gaacgagaagctgtacctgtactacctgcagaatggggggatatgtacgtggaccaggaactggacatcaaccggctgtccgactacga
tgtggacgctatcgtgcctcagagctttctgaaggacgactccatcgacaacaaggtgctgaccagaagcgacaagaaccggggcaaga
gcgacaacgtgccctccgaagaggtcgtgaagaagatgaagaactactggcggcagctgctgaacgccaagctgattacccagagaaa
gttcgacaatctgaccaaggccgagagaggcggcctgagcgaactggataaggccggcttcatcaagagacagctggtggaaacccgg
cagatcacaaagcacgtggcacagatcctggactcccggatgaacactaagtacgacgagaatgacaagctgatccgggaagtgaaagt
gatcaccctgaagtccaagctggtgtccgatttccggaaggatttccagttttacaaagtgcgcgagatcaacaactaccaccacgcccacg
acgcctacctgaacgccgtcgtgggaaccgccctgatcaaaaagtaccctaagctggaaagcgagttcgtgtacggcgactacaaggtgt
acgacgtgcggaagatgatcgccaagagcgagcaggaaatcggcaaggctaccgccaagtacttcttctacagcaacatcatgaacttttt
caagaccgagattaccctggccaacggcgagatccggaagcggcctctgatcgagacaaacggcgaaaccggggagatcgtgtggga
taagggccgggattttgccaccgtgcggaaagtgctgagcatgccccaagtgaatatcgtgaaaaagaccgaggtgcagacaggcggct
tcagcaaagagtctatcctgcccaagaggaacagcgataagctgatcgccagaaagaaggactgggaccctaagaagtacggcggcttc
gacagccccaccgtggcctattctgtgctggtggtggccaaagtggaaaagggcaagtccaagaaactgaagagtgtgaaagagctgct
ggggatcaccatcatggaaagaagcagcttcgagaagaatcccatcgactttctggaagccaagggctacaaagaagtgaaaaaggacc
tgatcatcaagctgcctaagtactccctgttcgagctggaaaacggccggaagagaatgctggcctctgccggcgaactgcagaagggaa
acgaactggccctgccctccaaatatgtgaacttcctgtacctggccagccactatgagaagctgaagggctcccccgaggataatgagca
gaaacagctgtttgtggaacagcacaagcactacctggacgagatcatcgagcagatcagcgagttctccaagagagtgatcctggccga
cgctaatctggacaaagtgctgtccgcctacaacaagcaccgggataagcccatcagagagcaggccgagaatatcatccacctgtttac
cctgaccaatctgggagcccctgccgccttcaagtactttgacaccaccatcgaccggaagaggtacaccagcaccaaagaggtgctgg
acgccaccctgatccaccagagcatcaccggcctgtacgagacacggatcgacctgtctcagctgggaggcgacaaaaggccggcgg
ccacgaaaaaggccggacaggccaaaaagaaaaagctcgagggcggaggcgggagcggatccccctcccggctccagatgttcttcg
ctaataaccacgaccaggaatttgaccctccaaaggtttacccacctgtcccagctgagaagaggaagcccatccgggtgctgtctctcttt
gatggaatcgctacagggctcctggtgctgaaggacttgggcattcaggtggaccgctacattgcctcggaggtgtgtgaggactccatca
cggtgggcatggtgcggcaccaggggaagatcatgtacgtcggggacgtccgcagcgtcacacagaagcatatccaggagtggggcc
cattcgatctggtgattgggggcagtccctgcaatgacctctccatcgtcaaccctgctcgcaagggcctctacgagggcactggccggct
cttctttgagttctaccgcctcctgcatgatgcgcggcccaaggagggagatgatcgccccttcttctggctctttgagaatgtggtggccatg
ggcgttagtgacaagagggacatctcgcgatttctcgagtccaaccctgtgatgattgatgccaaagaagtgtcagctgcacacagggccc
gctacttctggggtaaccttcccggtatgaacaggccgttggcatccactgtgaatgataagctggagctgcaggagtgtctggagcatgg
caggatagccaagttcagcaaagtgaggaccattactacgaggtcaaactccataaagcagggcaaagaccagcattttcctgtgttcatg
aatgagaaagaggacatcttatggtgcactgaaatggaaagggtatttggtttcccagtccactatactgacgtgtccaacatgagccgcttg
gcgaggcagagactgctgggccggtcatggagcgtgccagtcatccgccacctcttcgctccgctgaaggagtattttgcgtgtgtgtccg
gccggcccggatccggcgcaacaaacttctctctgctgaaacaagccggagatgtcgaagagaatcctggaccgaccgagtacaagcc
cacggtgcgcctcgccacccgcgacgacgtccccagggccgtacgcaccctcgccgccgegttegccgactaccccgccacgcgcca
caccgtcgatccggaccgccacatcgagcgggtcaccgagctgcaagaactcttcctcacgcgcgtcgggctcgacatcggcaaggtgt
gggtcgcggacgacggcgccgcggtggcggtctggaccacgccggagagcgtcgaagcgggggcggtgttcgccgagatcggccc
gcgcatggccgagttgagcggttcccggctggccgcgcagcaacagatggaaggcctcctggcgccgcaccggcccaaggagcccg
cgtggttcctggccaccgtcggagtctcgcccgaccaccagggcaagggtctgggcagcgccgtcgtgctccccggagtggaggcgg
ccgagcgcgccggggtgcccgccttcctggagacctccgcgccccgcaacctccccttctacgageggctcggcttcaccgtcaccgcc
gacgtcgaggtgcccgaaggaccgcgcacctggtgcatgacccgcaagcccggtgcctgaacgcgttaagtcgacaatcaacctctggatt
acaaaatttgtgaaagattgactggtattcttaactatgttgctccttttacgctatgtggatacgctgctttaatgcctttgtatcatgctattgc
ttcccgtatggctttcattttctcctccttgtataaatcctggttgctgtctctttatgaggagttgtggcccgttgtcaggcaacgtggcgtggt
gtgcactgtgtttgctgacgcaacccccactggttggggcattgccaccacctgtcagctcctttccgggactttcgctttccccctccctattgcc
acggcggaactcatcgccgcctgccttgcccgctgctggacaggggctcggctgttgggcactgacaattccgtggtgttgtcggggaaa
tcatcgtcctttccttggctgctcgcctgtgttgccacctggattctgcgcgggacgtccttctgctacgtcccttcggccctcaatccagcgg
accttccttcccgcggcctgctgccggctctgcggcctcttccgcgtcttcgccttcgccctcagacgagtcggatctccctttgggccgcct
ccccgcgtcgactttaagaccaatgacttacaaggcagctgtagatcttagccactttttaaaagaaaaggggggactggaagggctaattc
actcccaacgaagacaagatctgctttttgcttgtactgggtctctctggttagaccagatctgagcctgggagctctctggctaactagggaa
cccactgcttaagcctcaataaagcttgccttgagtgcttcaagtagtgtgtgcccgtctgttgtgtgactctggtaactagagatccctcagac
ccttttagtcagtgtggaaaatctctagcagggcccgtttaaacccgctgatcagcctcgactgtgccttctagttgccagccatctgttgtttg
cccctcccccgtgccttccttgaccctggaaggtgccactcccactgtcctttcctaataaaatgaggaaattgcatcgcattgtctgagtagg
tgtcattctattctggggggtggggggggcaggacagcaagggggaggattgggaagacaatagcaggcatgctggggatgcggtgg
gctctatggcttctgaggcggaaagaaccagctggggctctagggggtatccccacgcgccctgtagcggcgcattaagcgcggcgggt
gtggtggttacgcgcagcgtgaccgctacacttgccagcgccctagcgcccgctcctttcgctttcttcccttcctttctcgccacgttcgccg
gctttccccgtcaagctctaaatcgggggctccctttagggttccgatttagtgctttacggcacctcgaccccaaaaaacttgattagggtga
tggttcacgtagtgggccatcgccctgatagacggtttttcgccctttgacgttggagtccacgttctttaatagtggactcttgttccaaactgg
aacaacactcaaccctatctcggtctattcttttgatttataagggattttgccgatttcggcctattggttaaaaaatgagctgatttaacaaaa
atttaacgcgaattaattctgtggaatgtgtgtcagttagggtgtggaaagtccccaggctccccagcaggcagaagtatgcaaagcatgcatc
tcaattagtcagcaaccaggtgtggaaagtccccaggctccccagcaggcagaagtatgcaaagcatgcatctcaattagtcagcaaccat
agtcccgcccctaactccgcccatcccgcccctaactccgcccagttccgcccattctccgccccatggctgactaattttttttatttatgcag
aggccgaggccgcctctgcctctgagctattccagaagtagtgaggaggcttttttggaggcctaggcttttgcaaaaagctcccgggagc
ttgtatatccattttcggatctgatcagcacgtgttgacaattaatcatcggcatagtatatcggcatagtataatacgacaaggtgaggaacta
aaccatggccaagttgaccagtgccgttccggtgctcaccgcgcgcgacgtcgccggagcggtcgagttctggaccgaccggctcgggt
tctcccgggacttcgtggaggacgacttcgccggtgtggtccgggacgacgtgaccctgttcatcagcgcggtccaggaccaggtggtg
ccggacaacaccctggcctgggtgtgggtgcgcggcctggacgagctgtacgccgagtggtcggaggtcgtgtccacgaacttccggg
acgcctccgggccggccatgaccgagatcggcgagcagccgtggggggggagttcgccctgcgcgacccggccggcaactgcgtg
cacttcgtggccgaggagcaggactgacacgtgctacgagatttcgattccaccgccgccttctatgaaaggttgggcttcggaatcgttttc
cgggacgccggctggatgatcctccagcgcggggatctcatgctggagttcttcgcccaccccaacttgtttattgcagcttataatggttacaaa
taaagcaatagcatcacaaatttcacaaataaagcatttttttcactgcattctagttgtggtttgtccaaactcatcaatgtatcttatcatgtc
tgtataccgtcgacctctagctagagcttggcgtaatcatggtcatagctgtttcctgtgtgaaattgttatccgctcacaattccacacaacata
cgagccggaagcataaagtgtaaagcctggggtgcctaatgagtgagctaactcacattaattgcgttgcgctcactgcccgctttccagtc
gggaaacctgtcgtgccagctgcattaatgaatcggccaacgcgcggggagaggcggtttgcgtattgggcgctcttccgcttcctcgctc
actgactcgctgcgctcggtcgttcggctgcggcgagcggtatcagctcactcaaaggcggtaatacggttatccacagaatcaggggata
acgcaggaaagaacatgtgagcaaaaggccagcaaaaggccaggaaccgtaaaaaggccgcgttgctggcgtttttccataggctccgc
ccccctgacgagcatcacaaaaatcgacgctcaagtcagaggtggcgaaacccgacaggactataaagataccaggcgtttccccctgg
aagctccctcgtgcgctctcctgttccgaccctgccgcttaccggatacctgtccgcctttctcccttcgggaagcgtggcgctttctcatagc
tcacgctgtaggtatctcagttcggtgtaggtcgttcgctccaagctgggctgtgtgcacgaaccccccgttcagcccgaccgctgcgcctt
atccggtaactatcgtcttgagtccaacccggtaagacacgacttatcgccactggcagcagccactggtaacaggattagcagagcgag
gtatgtaggcggtgctacagagttcttgaagtggtggcctaactacggctacactagaagaacagtatttggtatctgcgctctgctgaagcc
agttaccttcggaaaaagagttggtagctcttgatccggcaaacaaaccaccgctggtagcggtggtttttttgtttgcaagcagcagattac
gcgcagaaaaaaaggatctcaagaagatcctttgatcttttctacggggtctgacgctcagtggaacgaaaactcacgttaagggattttggt
catgagattatcaaaaaggatcttcacctagatccttttaaattaaaaatgaagttttaaatcaatctaaagtatatatgagtaaacttggtctgac
agttaccaatgcttaatcagtgaggcacctatctcagcgatctgtctatttcgttcatccatagttgcctgactccccgtcgtgtagataactacg
atacgggagggcttaccatctggccccagtgctgcaatgataccgcgagacccacgctcaccggctccagatttatcagcaataaaccag
ccagccggaagggccgagcgcagaagtggtcctgcaactttatccgcctccatccagtctattaattgttgccgggaagctagagtaagta
gttcgccagttaatagtttgcgcaacgttgttgccattgctacaggcatcgtggtgtcacgctcgtcgtttggtatggcttcattcagctccggtt
cccaacgatcaaggcgagttacatgatcccccatgttgtgcaaaaaagcggttagctccttcggtcctccgatcgttgtcagaagtaagttgg
ccgcagtgttatcactcatggttatggcagcactgcataattctcttactgtcatgccatccgtaagatgcttttctgtgactggtgagtactcaa
ccaagtcattctgagaatagtgtatgcggcgaccgagttgctcttgcccggcgtcaatacgggataataccgcgccacatagcagaacttta
aaagtgctcatcattggaaaacgttcttcggggcgaaaactctcaaggatcttaccgctgttgagatccagttcgatgtaacccactcgtgca
cccaactgatcttcagcatcttttactttcaccagcgtttctgggtgagcaaaaacaggaaggcaaaatgccgcaaaaaagggaataaggg
cgacacggaaatgttgaatactcatactcttcctttttcaatattattgaagcatttatcagggttattgtctcatgagcggatacatatttgaat
gtatttagaaaaataaacaaataggggttccgcgcacatttccccgaaaagtgccacctgac.

In an aspect, a disclosed pBK1028 plasmid can comprise the sequence set forth below or a sequence having 70%, 75%, 80%, 85%, 90%, 95%, or more identity to the sequence set forth below:

(SEQ ID NO: 31)
gtcgacggatcgggagatctcccgatcccctatggtgcactctcagtacaatctgctctgatgccgcatagttaagccagtatctgctccctg
cttgtgtgttggaggtcgctgagtagtgcgcgagcaaaatttaagctacaacaaggcaaggcttgaccgacaattgcatgaagaatctgctt
agggttaggcgttttgcgctgcttcgcgatgtacgggccagatatacgcgttgacattgattattgactagttattaatagtaatcaattacggg
gtcattagttcatagcccatatatggagttccgcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtacatcaatgggcgtggata
gcggtttgactcacggggatttccaagtctccaccccattgacgtcaatgggagtttgttttggcaccaaaatcaacgggactttccaaaatgt
cgtaacaactccgccccattgacgcaaatgggcggtaggcgtgtacggtgggaggtctatataagcagcgcgttttgcctgtactgggtct
ctctggttagaccagatctgagcctgggagctctctggctaactagggaacccactgcttaagcctcaataaagcttgccttgagtgcttcaa
gtagtgtgtgcccgtctgttgtgtgactctggtaactagagatccctcagacccttttagtcagtgtggaaaatctctagcagtggcgcccgaa
cagggacttgaaagcgaaagggaaaccagaggagctctctcgacgcaggactcggcttgctgaagcgcgcacggcaagaggcgagg
ggcggcgactggtgagtacgccaaaaattttgactagcggaggctagaaggagagagatgggtgcgagagcgtcagtattaagcgggg
gagaattagatcgcgatgggaaaaaattcggttaaggccagggggaaagaaaaaatataaattaaaacatatagtatgggcaagcaggga
gctagaacgattcgcagttaatcctggcctgttagaaacatcagaaggctgtagacaaatactgggacagctacaaccatcccttcagacag
gatcagaagaacttagatcattatataatacagtagcaaccctctattgtgtgcatcaaaggatagagataaaagacaccaaggaagctttag
acaagatagaggaagagcaaaacaaaagtaagaccaccgcacagcaagcggccgctgatcttcagacctggaggaggagatatgagg
gacaattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatctgttgca
actcacagtctggggcatcaagcagctccaggcaagaatcctggctgtggaaagatacctaaaggatcaacagctcctggggatttgggg
ttgctctggaaaactcatttgcaccactgctgtgccttggaatgctagttggagtaataaatctctggaacagatttggaatcacacgacctgg
atggagtgggacagagaaattaacaattacacaagcttaatacactccttaattgaagaatcgcaaaaccagcaagaaaagaatgaacaag
aattattggaattagataaatgggcaagtttgtggaattggtttaacataacaaattggctgtggtatataaaattattcataatgatagtaggag
gcttggtaggtttaagaatagtttttgctgtactttctatagtgaatagagttaggcagggatattcaccattatcgtttcagacccacctcccaac
cccgaggggacccgacaggcccgaaggaatagaagaagaaggtggagagagagacagagacagatccattcgattagtgaacggatc
ggcactgcgtgcgccaattctgcagacaaatggcagtattcatccacaattttaaaagaaaaggggggattggggggtacagtgcagggg
aaagaatagtagacataatagcaacagacatacaaactaaagaattacaaaaacaaattacaaaaattcaaaattttcgggtttattacaggg
acagcagagatccagtttggttaattaatggggggacgttaacggggcggaacggtaccgagggcctatttcccatgattccttcatatttg
catatacgatacaaggctgttagagagataattagaattaatttgactgtaaacacaaagatattagtacaaaatacgtgacgtagaaagtaataat
ttcttgggtagtttgcagttttaaaattatgttttaaaatggactatcatatgcttaccgtaacttgaaagtatttcgatttcttggctttatatat
cttgtggaaaggacgaaacaccgactgggatgtaagccatagcgttttagagctagaaatagcaagttaaaataaggctagtccgttatcaact
tgaaaaagtggcaccgagtcggtgcttttttgaattcgctagctaggtcttgaaaggagtgggaattggctccggtgcccgtcagtgggcag
agcgcacatcgcccacagtccccgagaagttggggggaggggtcggcaattgatccggtgcctagagaaggtggcgcggggtaaact
gggaaagtgatgtcgtgtactggctccgcctttttcccgaggggggggagaaccgtatataagtgcagtagtcgccgtgaacgttctttttc
gcaacgggtttgccgccagaacacaggaccggtgccaccatggactataaggaccacgacggagactacaaggatcatgatattgattac
aaagacgatgacgataagatggccccaaagaagaagcggaaggtcggtatccacggagtcccagcagccgacaagaagtacagcatc
ggcctggccatcggcaccaactctgtgggctgggccgtgatcaccgacgagtacaaggtgcccagcaagaaattcaaggtgctgggcaa
caccgaccggcacagcatcaagaagaacctgatcggagccctgctgttcgacagcggcgaaacagccgaggccacccggctgaagag
aaccgccagaagaagatacaccagacggaagaaccggatctgctatctgcaagagatcttcagcaacgagatggccaaggtggacgac
agcttcttccacagactggaagagtccttcctggtggaagaggataagaagcacgagcggcaccccatcttcggcaacatcgtggacgag
gtggcctaccacgagaagtaccccaccatctaccacctgagaaagaaactggtggacagcaccgacaaggccgacctgcggctgatcta
tctggccctggcccacatgatcaagttccggggccacttcctgatcgagggcgacctgaaccccgacaacagcgacgtggacaagctgtt
catccagctggtgcagacctacaaccagctgttcgaggaaaaccccatcaacgccagcggcgtggacgccaaggccatcctgtctgcca
gactgagcaagagcagacggctggaaaatctgatcgcccagctgcccggcgagaagaagaatggcctgttcggcaacctgattgccctg
agcctgggcctgacccccaacttcaagagcaacttcgacctggccgaggatgccaaactgcagctgagcaaggacacctacgacgacg
acctggacaacctgctggcccagateggcgaccagtacgccgacctgtttctggccgccaagaacctgtccgacgccatcctgctgagcg
acatcctgagagtgaacaccgagatcaccaaggcccccctgagcgcctctatgatcaagagatacgacgagcaccaccaggacctgacc
ctgctgaaagctctcgtgcggcagcagctgcctgagaagtacaaagagattttcttcgaccagagcaagaacggctacgccggctacattg
acggcggagccagccaggaagagttctacaagttcatcaagcccatcctggaaaagatggacggcaccgaggaactgctcgtgaagct
gaacagagaggacctgctgcggaagcagcggaccttcgacaacggcagcatcccccaccagatccacctgggagagctgcacgccatt
ctgcggcggcaggaagatttttacccattcctgaaggacaaccgggaaaagatcgagaagatcctgaccttccgcatcccctactacgtgg
gccctctggccaggggaaacagcagattcgcctggatgaccagaaagagcgaggaaaccatcaccccctggaacttcgaggaagtggt
ggacaagggcgcttccgcccagagcttcatcgagcggatgaccaacttcgataagaacctgcccaacgagaaggtgctgcccaagcac
agcctgctgtacgagtacttcaccgtgtataacgagctgaccaaagtgaaatacgtgaccgagggaatgagaaagcccgccttcctgagc
ggcgagcagaaaaaggccatcgtggacctgctgttcaagaccaaccggaaagtgaccgtgaagcagctgaaagaggactacttcaaga
aaatcgagtgcttcgactccgtggaaatctccggcgtggaagatcggttcaacgcctccctgggcacataccacgatctgctgaaaattatc
aaggacaaggacttcctggacaatgaggaaaacgaggacattctggaagatatcgtgctgaccctgacactgtttgaggacagagagatg
atcgaggaacggctgaaaacctatgcccacctgttcgacgacaaagtgatgaagcagctgaagcggcggagatacaccggctggggca
ggctgagccggaagctgatcaacggcatccgggacaagcagtccggcaagacaatcctggatttcctgaagtccgacggcttcgccaac
agaaacttcatgcagctgatccacgacgacagcctgacctttaaagaggacatccagaaagcccaggtgtccggccagggcgatagcct
gcacgagcacattgccaatctggccggcagccccgccattaagaagggcatcctgcagacagtgaaggtggtggacgagctcgtgaaa
gtgatgggccggcacaagcccgagaacatcgtgatcgaaatggccagagagaaccagaccacccagaagggacagaagaacagccg
cgagagaatgaagcggatcgaagagggcatcaaagagctgggcagccagatcctgaaagaacaccccgtggaaaacacccagctgca
gaacgagaagctgtacctgtactacctgcagaatggggggatatgtacgtggaccaggaactggacatcaaccggctgtccgactacga
tgtggacgctatcgtgcctcagagctttctgaaggacgactccatcgacaacaaggtgctgaccagaagcgacaagaaccggggcaaga
gcgacaacgtgccctccgaagaggtcgtgaagaagatgaagaactactggcggcagctgctgaacgccaagctgattacccagagaaa
gttcgacaatctgaccaaggccgagagaggcggcctgagcgaactggataaggccggcttcatcaagagacagctggtggaaacccgg
cagatcacaaagcacgtggcacagatcctggactcccggatgaacactaagtacgacgagaatgacaagctgatccgggaagtgaaagt
gatcaccctgaagtccaagctggtgtccgatttccggaaggatttccagttttacaaagtgcgcgagatcaacaactaccaccacgcccacg
acgcctacctgaacgccgtcgtgggaaccgccctgatcaaaaagtaccctaagctggaaagcgagttcgtgtacggcgactacaaggtgt
acgacgtgcggaagatgatcgccaagagcgagcaggaaatcggcaaggctaccgccaagtacttcttctacagcaacatcatgaacttttt
caagaccgagattaccctggccaacggcgagatccggaagcggcctctgatcgagacaaacggcgaaaccggggagatcgtgtggga
taagggccgggattttgccaccgtgcggaaagtgctgagcatgccccaagtgaatatcgtgaaaaagaccgaggtgcagacaggcggct
tcagcaaagagtctatcctgcccaagaggaacagcgataagctgatcgccagaaagaaggactgggaccctaagaagtacggcggcttc
gacagccccaccgtggcctattctgtgctggtggtggccaaagtggaaaagggcaagtccaagaaactgaagagtgtgaaagagctgct
ggggatcaccatcatggaaagaagcagcttcgagaagaatcccatcgactttctggaagccaagggctacaaagaagtgaaaaaggacc
tgatcatcaagctgcctaagtactccctgttcgagctggaaaacggccggaagagaatgctggcctctgccggcgaactgcagaagggaa
acgaactggccctgccctccaaatatgtgaacttcctgtacctggccagccactatgagaagctgaagggctcccccgaggataatgagca
gaaacagctgtttgtggaacagcacaagcactacctggacgagatcatcgagcagatcagcgagttctccaagagagtgatcctggccga
cgctaatctggacaaagtgctgtccgcctacaacaagcaccgggataagcccatcagagagcaggccgagaatatcatccacctgtttac
cctgaccaatctgggagcccctgccgccttcaagtactttgacaccaccatcgaccggaagaggtacaccagcaccaaagaggtgctgg
acgccaccctgatccaccagagcatcaccggcctgtacgagacacggatcgacctgtctcagctgggaggcgacaaaaggccggcgg
ccacgaaaaaggccggacaggccaaaaagaaaaagctcgagggcggaggcgggagcggatccccctcccggctccagatgttcttcg
ctaataaccacgaccaggaatttgaccctccaaaggtttacccacctgtcccagctgagaagaggaagcccatccgggtgctgtctctcttt
gatggaatcgctacagggctcctggtgctgaaggacttgggcattcaggtggaccgctacattgcctcggaggtgtgtgaggactccatca
cggtgggcatggtgcggcaccaggggaagatcatgtacgtcggggacgtccgcagcgtcacacagaagcatatccaggagtggggcc
cattcgatctggtgattgggggcagtccctgcaatgacctctccatcgtcaaccctgctcgcaagggcctctacgagggcactggccggct
cttctttgagttctaccgcctcctgcatgatgcgcggcccaaggagggagatgatcgccccttcttctggctctttgagaatgtggtggccatg
ggcgttagtgacaagagggacatctcgcgatttctcgagtccaaccctgtgatgattgatgccaaagaagtgtcagctgcacacagggccc
gctacttctggggtaaccttcccggtatgaacaggccgttggcatccactgtgaatgataagctggagctgcaggagtgtctggagcatgg
caggatagccaagttcagcaaagtgaggaccattactacgaggtcaaactccataaagcagggcaaagaccagcattttcctgtgttcatg
aatgagaaagaggacatcttatggtgcactgaaatggaaagggtatttggtttcccagtccactatactgacgtgtccaacatgagccgcttg
gcgaggcagagactgctgggccggtcatggagcgtgccagtcatccgccacctcttcgctccgctgaaggagtattttgcgtgtgtgtccg
gccggcccggatccggcgcaacaaacttctctctgctgaaacaagccggagatgtcgaagagaatcctggaccgaccgagtacaagcc
cacggtgcgcctcgccacccgcgacgacgtccccagggccgtacgcaccctcgccgccgegttcgccgactaccccgccacgcgcca
caccgtcgatccggaccgccacatcgagcgggtcaccgagctgcaagaactcttcctcacgcgcgtcgggctcgacatcggcaaggtgt
gggtcgcggacgacggcgccgcggtggcggtctggaccacgccggagagcgtcgaagcgggggcggtgttcgccgagatcggccc
gcgcatggccgagttgagcggttcccggctggccgcgcagcaacagatggaaggcctcctggcgccgcaccggcccaaggagcccg
cgtggttcctggccaccgtcggagtctcgcccgaccaccagggcaagggtctgggcagcgccgtcgtgctccccggagtggaggcgg
ccgagcgcgccggggtgcccgccttcctggagacctccgcgccccgcaacctccccttctacgageggctcggcttcaccgtcaccgcc
gacgtcgaggtgcccgaaggaccgcgcacctggtgcatgacccgcaagcccggtgcctgaacgcgttaagtcgacaatcaacctctggatt
acaaaatttgtgaaagattgactggtattcttaactatgttgctccttttacgctatgtggatacgctgctttaatgcctttgtatcatgctattgc
ttcccgtatggctttcattttctcctccttgtataaatcctggttgctgtctctttatgaggagttgtggcccgttgtcaggcaacgtggcgtggtgt
gcactgtgtttgctgacgcaacccccactggttggggcattgccaccacctgtcagctcctttccgggactttcgctttccccctccctattgcc
acggcggaactcatcgccgcctgccttgcccgctgctggacaggggctcggctgttgggcactgacaattccgtggtgttgtcggggaaa
tcatcgtcctttccttggctgctcgcctgtgttgccacctggattctgcgcgggacgtccttctgctacgtcccttcggccctcaatccagcgg
accttccttcccgcggcctgctgccggctctgcggcctcttccgcgtcttcgccttcgccctcagacgagtcggatctccctttgggccgcct
ccccgcgtcgactttaagaccaatgacttacaaggcagctgtagatcttagccactttttaaaagaaaaggggggactggaagggctaattc
actcccaacgaagacaagatctgctttttgcttgtactgggtctctctggttagaccagatctgagcctgggagctctctggctaactagggaa
cccactgcttaagcctcaataaagcttgccttgagtgcttcaagtagtgtgtgcccgtctgttgtgtgactctggtaactagagatccctcagac
ccttttagtcagtgtggaaaatctctagcagggcccgtttaaacccgctgatcagcctcgactgtgccttctagttgccagccatctgttgtttg
cccctcccccgtgccttccttgaccctggaaggtgccactcccactgtcctttcctaataaaatgaggaaattgcatcgcattgtctgagtagg
tgtcattctattctggggggtggggggggcaggacagcaagggggaggattgggaagacaatagcaggcatgctggggatgcggtgg
gctctatggcttctgaggcggaaagaaccagctggggctctagggggtatccccacgcgccctgtagcggcgcattaagcgcggcgggt
gtggtggttacgcgcagcgtgaccgctacacttgccagcgccctagcgcccgctcctttcgctttcttcccttcctttctcgccacgttcgccg
gctttccccgtcaagctctaaatcgggggctccctttagggttccgatttagtgctttacggcacctcgaccccaaaaaacttgattagggtga
tggttcacgtagtgggccatcgccctgatagacggtttttcgccctttgacgttggagtccacgttctttaatagtggactcttgttccaaactgg
aacaacactcaaccctatctcggtctattcttttgatttataagggattttgccgatttcggcctattggttaaaaaatgagctgatttaacaaaaat
ttaacgcgaattaattctgtggaatgtgtgtcagttagggtgtggaaagtccccaggctccccagcaggcagaagtatgcaaagcatgcatc
tcaattagtcagcaaccaggtgtggaaagtccccaggctccccagcaggcagaagtatgcaaagcatgcatctcaattagtcagcaaccat
agtcccgcccctaactccgcccatcccgcccctaactccgcccagttccgcccattctccgccccatggctgactaattttttttatttatgcag
aggccgaggccgcctctgcctctgagctattccagaagtagtgaggaggcttttttggaggcctaggcttttgcaaaaagctcccgggagc
ttgtatatccattttcggatctgatcagcacgtgttgacaattaatcatcggcatagtatatcggcatagtataatacgacaaggtgaggaacta
aaccatggccaagttgaccagtgccgttccggtgctcaccgcgcgcgacgtcgccggagcggtcgagttctggaccgaccggctcgggt
tctcccgggacttcgtggaggacgacttcgccggtgtggtccgggacgacgtgaccctgttcatcagcgcggtccaggaccaggtggtg
ccggacaacaccctggcctgggtgtgggtgcgcggcctggacgagctgtacgccgagtggtcggaggtcgtgtccacgaacttccggg
acgcctccgggccggccatgaccgagatcggcgagcagccgtggggggggagttcgccctgcgcgacccggccggcaactgcgtg
cacttcgtggccgaggagcaggactgacacgtgctacgagatttcgattccaccgccgccttctatgaaaggttgggcttcggaatcgttttc
cgggacgccggctggatgatcctccagcgcggggatctcatgctggagttcttcgcccaccccaacttgtttattgcagcttataatggttacaaa
taaagcaatagcatcacaaatttcacaaataaagcatttttttcactgcattctagttgtggtttgtccaaactcatcaatgtatcttatcatgtc
tgtataccgtcgacctctagctagagcttggcgtaatcatggtcatagctgtttcctgtgtgaaattgttatccgctcacaattccacacaacata
cgagccggaagcataaagtgtaaagcctggggtgcctaatgagtgagctaactcacattaattgcgttgcgctcactgcccgctttccagtc
gggaaacctgtcgtgccagctgcattaatgaatcggccaacgcgcggggagaggcggtttgcgtattgggcgctcttccgcttcctcgctc
actgactcgctgcgctcggtcgttcggctgcggcgagcggtatcagctcactcaaaggcggtaatacggttatccacagaatcaggggata
acgcaggaaagaacatgtgagcaaaaggccagcaaaaggccaggaaccgtaaaaaggccgcgttgctggcgtttttccataggctccgc
ccccctgacgagcatcacaaaaatcgacgctcaagtcagaggtggcgaaacccgacaggactataaagataccaggcgtttccccctgg
aagctccctcgtgcgctctcctgttccgaccctgccgcttaccggatacctgtccgcctttctcccttcgggaagcgtggcgctttctcatagc
tcacgctgtaggtatctcagttcggtgtaggtcgttcgctccaagctgggctgtgtgcacgaaccccccgttcagcccgaccgctgcgcctt
atccggtaactatcgtcttgagtccaacccggtaagacacgacttatcgccactggcagcagccactggtaacaggattagcagagcgag
gtatgtaggcggtgctacagagttcttgaagtggtggcctaactacggctacactagaagaacagtatttggtatctgcgctctgctgaagcc
agttaccttcggaaaaagagttggtagctcttgatccggcaaacaaaccaccgctggtagcggtggtttttttgtttgcaagcagcagattac
gcgcagaaaaaaaggatctcaagaagatcctttgatcttttctacggggtctgacgctcagtggaacgaaaactcacgttaagggattttggt
catgagattatcaaaaaggatcttcacctagatccttttaaattaaaaatgaagttttaaatcaatctaaagtatatatgagtaaacttggtctgac
agttaccaatgcttaatcagtgaggcacctatctcagcgatctgtctatttcgttcatccatagttgcctgactccccgtcgtgtagataactacg
atacgggagggcttaccatctggccccagtgctgcaatgataccgcgagacccacgctcaccggctccagatttatcagcaataaaccag
ccagccggaagggccgagcgcagaagtggtcctgcaactttatccgcctccatccagtctattaattgttgccgggaagctagagtaagta
gttcgccagttaatagtttgcgcaacgttgttgccattgctacaggcatcgtggtgtcacgctcgtcgtttggtatggcttcattcagctccggtt
cccaacgatcaaggcgagttacatgatcccccatgttgtgcaaaaaagcggttagctccttcggtcctccgatcgttgtcagaagtaagttgg
ccgcagtgttatcactcatggttatggcagcactgcataattctcttactgtcatgccatccgtaagatgcttttctgtgactggtgagtactcaa
ccaagtcattctgagaatagtgtatgcggcgaccgagttgctcttgcccggcgtcaatacgggataataccgcgccacatagcagaacttta
aaagtgctcatcattggaaaacgttcttcggggcgaaaactctcaaggatcttaccgctgttgagatccagttcgatgtaacccactcgtgca
cccaactgatcttcagcatcttttactttcaccagcgtttctgggtgagcaaaaacaggaaggcaaaatgccgcaaaaaagggaataaggg
cgacacggaaatgttgaatactcatactcttcctttttcaatattattgaagcatttatcagggttattgtctcatgagcggatacatatttgaa
tgtatttagaaaaataaacaaataggggttccgcgcacatttccccgaaaagtgccacctgac.

In an aspect, a disclosed pBK1029 plasmid can comprise the sequence set forth below or a sequence having 70%, 75%, 80%, 85%, 90%, 95%, or more identity to the sequence set forth below:

(SEQ ID NO: 32)
gtcgacggatcgggagatctcccgatcccctatggtgcactctcagtacaatctgctctgatgccgcatagttaagccagtatctgctccctg
cttgtgtgttggaggtcgctgagtagtgcgcgagcaaaatttaagctacaacaaggcaaggcttgaccgacaattgcatgaagaatctgctt
agggttaggcgttttgcgctgcttcgcgatgtacgggccagatatacgcgttgacattgattattgactagttattaatagtaatcaattacggg
gtcattagttcatagcccatatatggagttccgcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtacatcaatgggcgtggata
gcggtttgactcacggggatttccaagtctccaccccattgacgtcaatgggagtttgttttggcaccaaaatcaacgggactttccaaaatgt
cgtaacaactccgccccattgacgcaaatgggcggtaggcgtgtacggtgggaggtctatataagcagcgcgttttgcctgtactgggtct
ctctggttagaccagatctgagcctgggagctctctggctaactagggaacccactgcttaagcctcaataaagcttgccttgagtgcttcaa
gtagtgtgtgcccgtctgttgtgtgactctggtaactagagatccctcagacccttttagtcagtgtggaaaatctctagcagtggcgcccgaa
cagggacttgaaagcgaaagggaaaccagaggagctctctcgacgcaggactcggcttgctgaagcgcgcacggcaagaggcgagg
ggcggcgactggtgagtacgccaaaaattttgactagcggaggctagaaggagagagatgggtgcgagagcgtcagtattaagcgggg
gagaattagatcgcgatgggaaaaaattcggttaaggccagggggaaagaaaaaatataaattaaaacatatagtatgggcaagcaggga
gctagaacgattcgcagttaatcctggcctgttagaaacatcagaaggctgtagacaaatactgggacagctacaaccatcccttcagacag
gatcagaagaacttagatcattatataatacagtagcaaccctctattgtgtgcatcaaaggatagagataaaagacaccaaggaagctttag
acaagatagaggaagagcaaaacaaaagtaagaccaccgcacagcaagcggccgctgatcttcagacctggaggaggagatatgagg
gacaattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatctgttgca
actcacagtctggggcatcaagcagctccaggcaagaatcctggctgtggaaagatacctaaaggatcaacagctcctggggatttgggg
ttgctctggaaaactcatttgcaccactgctgtgccttggaatgctagttggagtaataaatctctggaacagatttggaatcacacgacctgg
atggagtgggacagagaaattaacaattacacaagcttaatacactccttaattgaagaatcgcaaaaccagcaagaaaagaatgaacaag
aattattggaattagataaatgggcaagtttgtggaattggtttaacataacaaattggctgtggtatataaaattattcataatgatagtaggag
gcttggtaggtttaagaatagtttttgctgtactttctatagtgaatagagttaggcagggatattcaccattatcgtttcagacccacctcccaac
cccgaggggacccgacaggcccgaaggaatagaagaagaaggtggagagagagacagagacagatccattcgattagtgaacggatc
ggcactgcgtgcgccaattctgcagacaaatggcagtattcatccacaattttaaaagaaaaggggggattggggggtacagtgcagggg
aaagaatagtagacataatagcaacagacatacaaactaaagaattacaaaaacaaattacaaaaattcaaaattttcgggtttattacaggg
acagcagagatccagtttggttaattaatggggggacgttaacggggggaacggtaccgagggcctatttcccatgattccttcatatttg
catatacgatacaaggctgttagagagataattagaattaatttgactgtaaacacaaagatattagtacaaaatacgtgacgtagaaagtaataat
ttcttgggtagtttgcagttttaaaattatgttttaaaatggactatcatatgcttaccgtaacttgaaagtatttcgatttcttggctttatatat
cttgtggaaaggacgaaacaccggttggagcttagaatgtgaagttttagagctagaaatagcaagttaaaataaggctagtccgttatcaactt
gaaaaagtggcaccgagtcggtgcttttttgaattcgctagctaggtcttgaaaggagtgggaattggctccggtgcccgtcagtgggcag
agcgcacatcgcccacagtccccgagaagttggggggaggggtcggcaattgatccggtgcctagagaaggtggcgcggggtaaact
gggaaagtgatgtcgtgtactggctccgcctttttcccgaggggggggagaaccgtatataagtgcagtagtcgccgtgaacgttctttttc
gcaacgggtttgccgccagaacacaggaccggtgccaccatggactataaggaccacgacggagactacaaggatcatgatattgattac
aaagacgatgacgataagatggccccaaagaagaagcggaaggtcggtatccacggagtcccagcagccgacaagaagtacagcatc
ggcctggccatcggcaccaactctgtgggctgggccgtgatcaccgacgagtacaaggtgcccagcaagaaattcaaggtgctgggcaa
caccgaccggcacagcatcaagaagaacctgatcggagccctgctgttcgacagcggcgaaacagccgaggccacccggctgaagag
aaccgccagaagaagatacaccagacggaagaaccggatctgctatctgcaagagatcttcagcaacgagatggccaaggtggacgac
gtggcctaccacgagaagtaccccaccattaccacctgagaaagaaactggtggacagcaccgacaaggccgacctgcggctgatcta
tctggccctggcccacatgatcaagttccggggccacttcctgatcgagggcgacctgaaccccgacaacagcgacgtggacaagctgtt
catccagctggtgcagacctacaaccagctgttcgaggaaaaccccatcaacgccagcggcgtggacgccaaggccatcctgtctgcca
gactgagcaagagcagacggctggaaaatctgatcgcccagctgcccggcgagaagaagaatggcctgttcggcaacctgattgccctg
agcctgggcctgacccccaacttcaagagcaacttcgacctggccgaggatgccaaactgcagctgagcaaggacacctacgacgacg
acctggacaacctgctggcccagatcggcgaccagtacgccgacctgtttctggccgccaagaacctgtccgacgccatcctgctgagcg
acatcctgagagtgaacaccgagatcaccaaggcccccctgagcgcctctatgatcaagagatacgacgagcaccaccaggacctgacc
ctgctgaaagctctcgtgcggcagcagctgcctgagaagtacaaagagattttcttcgaccagagcaagaacggctacgccggctacattg
acggcggagccagccaggaagagttctacaagttcatcaagcccatcctggaaaagatggacggcaccgaggaactgctcgtgaagct
gaacagagaggacctgctgcggaagcagcggaccttcgacaacggcagcatcccccaccagatccacctgggagagctgcacgccatt
ctgcggcggcaggaagatttttacccattcctgaaggacaaccgggaaaagatcgagaagatcctgaccttccgcatcccctactacgtgg
gccctctggccaggggaaacagcagattcgcctggatgaccagaaagagcgaggaaaccatcaccccctggaacttcgaggaagtggt
ggacaagggcgcttccgcccagagcttcatcgagcggatgaccaacttcgataagaacctgcccaacgagaaggtgctgcccaagcac
agcctgctgtacgagtacttcaccgtgtataacgagctgaccaaagtgaaatacgtgaccgagggaatgagaaagcccgccttcctgagc
ggcgagcagaaaaaggccatcgtggacctgctgttcaagaccaaccggaaagtgaccgtgaagcagctgaaagaggactacttcaaga
aaatcgagtgcttcgactccgtggaaatctccggcgtggaagatcggttcaacgcctccctgggcacataccacgatctgctgaaaattatc
aaggacaaggacttcctggacaatgaggaaaacgaggacattctggaagatatcgtgctgaccctgacactgtttgaggacagagagatg
atcgaggaacggctgaaaacctatgcccacctgttcgacgacaaagtgatgaagcagctgaagcggcggagatacaccggctggggca
ggctgagccggaagctgatcaacggcatccgggacaagcagtccggcaagacaatcctggatttcctgaagtccgacggcttcgccaac
agaaacttcatgcagctgatccacgacgacagcctgacctttaaagaggacatccagaaagcccaggtgtccggccagggcgatagcct
gcacgagcacattgccaatctggccggcagccccgccattaagaagggcatcctgcagacagtgaaggtggtggacgagctcgtgaaa
gtgatgggccggcacaagcccgagaacatcgtgatcgaaatggccagagagaaccagaccacccagaagggacagaagaacagccg
cgagagaatgaagcggatcgaagagggcatcaaagagctgggcagccagatcctgaaagaacaccccgtggaaaacacccagctgca
gaacgagaagctgtacctgtactacctgcagaatggggggatatgtacgtggaccaggaactggacatcaaccggctgtccgactacga
tgtggacgctatcgtgcctcagagctttctgaaggacgactccatcgacaacaaggtgctgaccagaagcgacaagaaccggggcaaga
gcgacaacgtgccctccgaagaggtcgtgaagaagatgaagaactactggcggcagctgctgaacgccaagctgattacccagagaaa
gttcgacaatctgaccaaggccgagagaggcggcctgagcgaactggataaggccggcttcatcaagagacagctggtggaaacccgg
cagatcacaaagcacgtggcacagatcctggactcccggatgaacactaagtacgacgagaatgacaagctgatccgggaagtgaaagt
gatcaccctgaagtccaagctggtgtccgatttccggaaggatttccagttttacaaagtgcgcgagatcaacaactaccaccacgcccacg
acgcctacctgaacgccgtcgtgggaaccgccctgatcaaaaagtaccctaagctggaaagcgagttcgtgtacggcgactacaaggtgt
acgacgtgcggaagatgatcgccaagagcgagcaggaaatcggcaaggctaccgccaagtacttcttctacagcaacatcatgaacttttt
caagaccgagattaccctggccaacggcgagatccggaagcggcctctgatcgagacaaacggcgaaaccggggagatcgtgtggga
taagggccgggattttgccaccgtgcggaaagtgctgagcatgccccaagtgaatatcgtgaaaaagaccgaggtgcagacaggcggct
tcagcaaagagtctatcctgcccaagaggaacagcgataagctgatcgccagaaagaaggactgggaccctaagaagtacggcggcttc
gacagccccaccgtggcctattctgtgctggtggtggccaaagtggaaaagggcaagtccaagaaactgaagagtgtgaaagagctgct
ggggatcaccatcatggaaagaagcagcttcgagaagaatcccatcgactttctggaagccaagggctacaaagaagtgaaaaaggacc
tgatcatcaagctgcctaagtactccctgttcgagctggaaaacggccggaagagaatgctggcctctgccggcgaactgcagaagggaa
acgaactggccctgccctccaaatatgtgaacttcctgtacctggccagccactatgagaagctgaagggctcccccgaggataatgagca
gaaacagctgtttgtggaacagcacaagcactacctggacgagatcatcgagcagatcagcgagttctccaagagagtgatcctggccga
cgctaatctggacaaagtgctgtccgcctacaacaagcaccgggataagcccatcagagagcaggccgagaatatcatccacctgtttac
cctgaccaatctgggagcccctgccgccttcaagtactttgacaccaccatcgaccggaagaggtacaccagcaccaaagaggtgctgg
acgccaccctgatccaccagagcatcaccggcctgtacgagacacggatcgacctgtctcagctgggaggcgacaaaaggccggcgg
ccacgaaaaaggccggacaggccaaaaagaaaaagctcgagggcggaggcgggagcggatccccctcccggctccagatgttcttcg
ctaataaccacgaccaggaatttgaccctccaaaggtttacccacctgtcccagctgagaagaggaagcccatccgggtgctgtctctcttt
gatggaatcgctacagggctcctggtgctgaaggacttgggcattcaggtggaccgctacattgcctcggaggtgtgtgaggactccatca
cggtgggcatggtgcggcaccaggggaagatcatgtacgtcggggacgtccgcagcgtcacacagaagcatatccaggagtggggcc
cattcgatctggtgattgggggcagtccctgcaatgacctctccatcgtcaaccctgctcgcaagggcctctacgagggcactggccggct
cttctttgagttctaccgcctcctgcatgatgcgcggcccaaggagggagatgatcgccccttcttctggctctttgagaatgtggtggccatg
ggcgttagtgacaagagggacatctcgcgatttctcgagtccaaccctgtgatgattgatgccaaagaagtgtcagctgcacacagggccc
gctacttctggggtaaccttcccggtatgaacaggccgttggcatccactgtgaatgataagctggagctgcaggagtgtctggagcatgg
caggatagccaagttcagcaaagtgaggaccattactacgaggtcaaactccataaagcagggcaaagaccagcattttcctgtgttcatg
aatgagaaagaggacatcttatggtgcactgaaatggaaagggtatttggtttcccagtccactatactgacgtgtccaacatgagccgcttg
gcgaggcagagactgctgggccggtcatggagcgtgccagtcatccgccacctcttcgctccgctgaaggagtattttgcgtgtgtgtccg
gccggcccggatccggcgcaacaaacttctctctgctgaaacaagccggagatgtcgaagagaatcctggaccgaccgagtacaagcc
cacggtgcgcctcgccacccgcgacgacgtccccagggccgtacgcaccctcgccgccgegttcgccgactaccccgccacgcgcca
caccgtcgatccggaccgccacatcgagcgggtcaccgagctgcaagaactcttcctcacgcgcgtcgggctcgacatcggcaaggtgt
gggtcgcggacgacggcgccgcggtggcggtctggaccacgccggagagcgtcgaagcgggggcggtgttcgccgagatcggccc
gcgcatggccgagttgagcggttcccggctggccgcgcagcaacagatggaaggcctcctggcgccgcaccggcccaaggagcccg
cgtggttcctggccaccgtcggagtctcgcccgaccaccagggcaagggtctgggcagcgccgtcgtgctccccggagtggaggcgg
ccgagcgcgccggggtgcccgccttcctggagacctccgcgccccgcaacctccccttctacgageggcteggcttcaccgtcaccgcc
gacgtcgaggtgcccgaaggaccgcgcacctggtgcatgacccgcaagcccggtgcctgaacgcgttaagtcgacaatcaacctctggatt
acaaaatttgtgaaagattgactggtattcttaactatgttgctccttttacgctatgtggatacgctgctttaatgcctttgtatcatgctattgc
ttcccgtatggctttcattttctcctccttgtataaatcctggttgctgtctctttatgaggagttgtggcccgttgtcaggcaacgtggcgtggt
gcactgtgtttgctgacgcaacccccactggttggggcattgccaccacctgtcagctcctttccgggactttcgetttccccctccctattgcc
gtacggcggaactcatcgccgcctgccttgcccgctgctggacaggggctcggctgttgggcactgacaattccgtggtgttgtcggggaaa
tcatcgtcctttccttggctgctcgcctgtgttgccacctggattctgcgcgggacgtccttctgctacgtcccttcggccctcaatccagcgg
accttccttcccgcggcctgctgccggctctgcggcctcttccgcgtcttcgccttcgccctcagacgagtcggatctccctttgggccgcct
ccccgcgtcgactttaagaccaatgacttacaaggcagctgtagatcttagccactttttaaaagaaaaggggggactggaagggctaattc
actcccaacgaagacaagatctgctttttgcttgtactgggtctctctggttagaccagatctgagcctgggagctctctggctaactagggaa
cccactgcttaagcctcaataaagcttgccttgagtgcttcaagtagtgtgtgcccgtctgttgtgtgactctggtaactagagatccctcagac
ccttttagtcagtgtggaaaatctctagcagggcccgtttaaacccgctgatcagcctcgactgtgccttctagttgccagccatctgttgtttg
cccctcccccgtgccttccttgaccctggaaggtgccactcccactgtcctttcctaataaaatgaggaaattgcatcgcattgtctgagtagg
tgtcattctattctggggggtggggggggcaggacagcaagggggaggattgggaagacaatagcaggcatgctggggatgcggtgg
gctctatggcttctgaggcggaaagaaccagctggggctctagggggtatccccacgcgccctgtagcggcgcattaagcgcggcgggt
gtggtggttacgcgcagcgtgaccgctacacttgccagcgccctagcgcccgctcctttcgctttcttcccttcctttctcgccacgttcgccg
gctttccccgtcaagctctaaatcgggggctccctttagggttccgatttagtgctttacggcacctcgaccccaaaaaacttgattagggtga
tggttcacgtagtgggccatcgccctgatagacggtttttcgccctttgacgttggagtccacgttctttaatagtggactcttgttccaaactgg
aacaacactcaaccctatctcggtctattcttttgatttataagggattttgccgatttcggcctattggttaaaaaatgagctgatttaacaaaa
atttaacgcgaattaattctgtggaatgtgtgtcagttagggtgtggaaagtccccaggctccccagcaggcagaagtatgcaaagcatgcatc
tcaattagtcagcaaccaggtgtggaaagtccccaggctccccagcaggcagaagtatgcaaagcatgcatctcaattagtcagcaaccat
agtcccgcccctaactccgcccatcccgcccctaactccgcccagttccgcccattctccgccccatggctgactaattttttttatttatgcag
aggccgaggccgcctctgcctctgagctattccagaagtagtgaggaggcttttttggaggcctaggcttttgcaaaaagctcccgggagc
ttgtatatccattttcggatctgatcagcacgtgttgacaattaatcatcggcatagtatatcggcatagtataatacgacaaggtgaggaacta
aaccatggccaagttgaccagtgccgttccggtgctcaccgcgcgcgacgtcgccggagcggtcgagttctggaccgaccggctcgggt
tctcccgggacttcgtggaggacgacttcgccggtgtggtccgggacgacgtgaccctgttcatcagcgcggtccaggaccaggtggtg
ccggacaacaccctggcctgggtgtgggtgcgcggcctggacgagctgtacgccgagtggtcggaggtcgtgtccacgaacttccggg
acgcctccgggccggccatgaccgagatcggcgagcagccgtggggggggagttcgccctgcgcgacccggccggcaactgcgtg
cacttcgtggccgaggagcaggactgacacgtgctacgagatttcgattccaccgccgccttctatgaaaggttgggcttcggaatcgttttc
cgggacgccggctggatgatcctccagcgcggggatctcatgctggagttcttcgcccaccccaacttgtttattgcagcttataatggttacaaa
taaagcaatagcatcacaaatttcacaaataaagcatttttttcactgcattctagttgtggtttgtccaaactcatcaatgtatcttatcatgtc
tgtataccgtcgacctctagctagagcttggcgtaatcatggtcatagctgtttcctgtgtgaaattgttatccgctcacaattccacacaacata
cgagccggaagcataaagtgtaaagcctggggtgcctaatgagtgagctaactcacattaattgcgttgcgctcactgcccgctttccagtc
gggaaacctgtcgtgccagctgcattaatgaatcggccaacgcgcggggagaggcggtttgcgtattgggcgctcttccgcttcctcgctc
actgactcgctgcgctcggtcgttcggctgcggcgagcggtatcagctcactcaaaggcggtaatacggttatccacagaatcaggggata
acgcaggaaagaacatgtgagcaaaaggccagcaaaaggccaggaaccgtaaaaaggccgcgttgctggcgtttttccataggctccgc
ccccctgacgagcatcacaaaaatcgacgctcaagtcagaggtggcgaaacccgacaggactataaagataccaggcgtttccccctgg
aagctccctcgtgcgctctcctgttccgaccctgccgcttaccggatacctgtccgcctttctcccttcgggaagcgtggcgctttctcatagc
tcacgctgtaggtatctcagttcggtgtaggtcgttcgctccaagctgggctgtgtgcacgaaccccccgttcagcccgaccgctgcgcctt
atccggtaactatcgtcttgagtccaacccggtaagacacgacttatcgccactggcagcagccactggtaacaggattagcagagcgag
gtatgtaggcggtgctacagagttcttgaagtggtggcctaactacggctacactagaagaacagtatttggtatctgcgctctgctgaagcc
agttaccttcggaaaaagagttggtagctcttgatccggcaaacaaaccaccgctggtagcggtggtttttttgtttgcaagcagcagattac
gcgcagaaaaaaaggatctcaagaagatcctttgatcttttctacggggtctgacgctcagtggaacgaaaactcacgttaagggattttggt
catgagattatcaaaaaggatcttcacctagatccttttaaattaaaaatgaagttttaaatcaatctaaagtatatatgagtaaacttggtctgac
agttaccaatgcttaatcagtgaggcacctatctcagcgatctgtctatttcgttcatccatagttgcctgactccccgtcgtgtagataactacg
atacgggagggcttaccatctggccccagtgctgcaatgataccgcgagacccacgctcaccggctccagatttatcagcaataaaccag
ccagccggaagggccgagcgcagaagtggtcctgcaactttatccgcctccatccagtctattaattgttgccgggaagctagagtaagta
gttcgccagttaatagtttgcgcaacgttgttgccattgctacaggcatcgtggtgtcacgctcgtcgtttggtatggcttcattcagctccggtt
cccaacgatcaaggcgagttacatgatcccccatgttgtgcaaaaaagcggttagctccttcggtcctccgatcgttgtcagaagtaagttgg
ccgcagtgttatcactcatggttatggcagcactgcataattctcttactgtcatgccatccgtaagatgcttttctgtgactggtgagtactcaa
ccaagtcattctgagaatagtgtatgcggcgaccgagttgctcttgcccggcgtcaatacgggataataccgcgccacatagcagaacttta
aaagtgctcatcattggaaaacgttcttcggggcgaaaactctcaaggatcttaccgctgttgagatccagttcgatgtaacccactcgtgca
cccaactgatcttcagcatcttttactttcaccagcgtttctgggtgagcaaaaacaggaaggcaaaatgccgcaaaaaagggaataaggg
cgacacggaaatgttgaatactcatactcttcctttttcaatattattgaagcatttatcagggttattgtctcatgagcggatacatatttgaa
tgtatttagaaaaataaacaaataggggttccgcgcacatttccccgaaaagtgccacctgac.

In an aspect, a disclosed pBK1030 plasmid can comprise the sequence set forth below or a sequence having 70%, 75%, 80%, 85%, 90%, 95%, or more identity to the sequence set forth

(SEQ ID NO: 33)
gtcgacggatcgggagatctcccgatcccctatggtgcactctcagtacaatctgctctgatgccgcatagttaagccagtatctgctccctg
cttgtgtgttggaggtcgctgagtagtgcgcgagcaaaatttaagctacaacaaggcaaggcttgaccgacaattgcatgaagaatctgctt
agggttaggcgttttgcgctgcttcgcgatgtacgggccagatatacgcgttgacattgattattgactagttattaatagtaatcaattacggg
gtcattagttcatagcccatatatggagttccgcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtacatcaatgggcgtggata
gcggtttgactcacggggatttccaagtctccaccccattgacgtcaatgggagtttgttttggcaccaaaatcaacgggactttccaaaatgt
cgtaacaactccgccccattgacgcaaatgggcggtaggcgtgtacggtgggaggtctatataagcagcgcgttttgcctgtactgggtct
ctctggttagaccagatctgagcctgggagctctctggctaactagggaacccactgcttaagcctcaataaagcttgccttgagtgcttcaa
gtagtgtgtgcccgtctgttgtgtgactctggtaactagagatccctcagacccttttagtcagtgtggaaaatctctagcagtggcgcccgaa
cagggacttgaaagcgaaagggaaaccagaggagctctctcgacgcaggactcggcttgctgaagcgcgcacggcaagaggcgagg
ggcggcgactggtgagtacgccaaaaattttgactagcggaggctagaaggagagagatgggtgcgagagcgtcagtattaagcgggg
gagaattagatcgcgatgggaaaaaattcggttaaggccagggggaaagaaaaaatataaattaaaacatatagtatgggcaagcaggga
gctagaacgattcgcagttaatcctggcctgttagaaacatcagaaggctgtagacaaatactgggacagctacaaccatcccttcagacag
gatcagaagaacttagatcattatataatacagtagcaaccctctattgtgtgcatcaaaggatagagataaaagacaccaaggaagctttag
acaagatagaggaagagcaaaacaaaagtaagaccaccgcacagcaagcggccgctgatcttcagacctggaggaggagatatgagg
gacaattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatctgttgca
actcacagtctggggcatcaagcagctccaggcaagaatcctggctgtggaaagatacctaaaggatcaacagctcctggggatttgggg
ttgctctggaaaactcatttgcaccactgctgtgccttggaatgctagttggagtaataaatctctggaacagatttggaatcacacgacctgg
atggagtgggacagagaaattaacaattacacaagcttaatacactccttaattgaagaatcgcaaaaccagcaagaaaagaatgaacaag
aattattggaattagataaatgggcaagtttgtggaattggtttaacataacaaattggctgtggtatataaaattattcataatgatagtaggag
gcttggtaggtttaagaatagtttttgctgtactttctatagtgaatagagttaggcagggatattcaccattatgtttcagacccacctcccaac
cccgaggggacccgacaggcccgaaggaatagaagaagaaggtggagagagagacagagacagatccattcgattagtgaacggatc
ggcactgcgtgcgccaattctgcagacaaatggcagtattcatccacaattttaaaagaaaaggggggattggggggtacagtgcagggg
aaagaatagtagacataatagcaacagacatacaaactaaagaattacaaaaacaaattacaaaaattcaaaattttcgggtttattacaggg
acagcagagatccagtttggttaattaatgggcgggacgttaacggggcggaacggtaccgagggcctatttcccatgattccttcatatttg
catatacgatacaaggctgttagagagataattagaattaatttgactgtaaacacaaagatattagtacaaaatacgtgacgtagaaagtaataat
ttcttgggtagtttgcagttttaaaattatgttttaaaatggactatcatatgcttaccgtaacttgaaagtatttcgatttcttggctttatatatct
tgtggaaaggacgaaacaccggacagggggagccctataatgttttagagctagaaatagcaagttaaaataaggctagtccgttatcaac
ttgaaaaagtggcaccgagtcggtgcttttttgaattcgctagctaggtcttgaaaggagtgggaattggctccggtgcccgtcagtgggca
gagcgcacatcgcccacagtccccgagaagttggggggaggggtcggcaattgatccggtgcctagagaaggtggcgcggggtaaac
tgggaaagtgatgtcgtgtactggctccgcctttttcccgaggggggggagaaccgtatataagtgcagtagtcgccgtgaacgttctttttc
gcaacgggtttgccgccagaacacaggaccggtgccaccatggactataaggaccacgacggagactacaaggatcatgatattgattac
aaagacgatgacgataagatggccccaaagaagaagcggaaggtcggtatccacggagtcccagcagccgacaagaagtacagcatc
ggcctggccatcggcaccaactctgtgggctgggccgtgatcaccgacgagtacaaggtgcccagcaagaaattcaaggtgctgggcaa
caccgaccggcacagcatcaagaagaacctgatcggagccctgctgttcgacagcggcgaaacagccgaggccacccggctgaagag
aaccgccagaagaagatacaccagacggaagaaccggatctgctatctgcaagagatcttcagcaacgagatggccaaggtggacgac
agcttcttccacagactggaagagtccttcctggtggaagaggataagaagcacgagcggcaccccatcttcggcaacatcgtggacgag
gtggcctaccacgagaagtaccccaccatctaccacctgagaaagaaactggtggacagcaccgacaaggccgacctgcggctgatcta
tctggccctggcccacatgatcaagttccggggccacttcctgatcgagggcgacctgaaccccgacaacagcgacgtggacaagctgtt
catccagctggtgcagacctacaaccagctgttcgaggaaaaccccatcaacgccagcggcgtggacgccaaggccatcctgtctgcca
gactgagcaagagcagacggctggaaaatctgatcgcccagctgcccggcgagaagaagaatggcctgttcggcaacctgattgccctg
agcctgggcctgacccccaacttcaagagcaacttcgacctggccgaggatgccaaactgcagctgagcaaggacacctacgacgacg
acctggacaacctgctggcccagateggcgaccagtacgccgacctgtttctggccgccaagaacctgtccgacgccatcctgctgagcg
acatcctgagagtgaacaccgagatcaccaaggcccccctgagcgcctctatgatcaagagatacgacgagcaccaccaggacctgacc
ctgctgaaagctctcgtgcggcagcagctgcctgagaagtacaaagagattttcttcgaccagagcaagaacggctacgccggctacattg
acggcggagccagccaggaagagttctacaagttcatcaagcccatcctggaaaagatggacggcaccgaggaactgctcgtgaagct
gaacagagaggacctgctgcggaagcagcggaccttcgacaacggcagcatcccccaccagatccacctgggagagctgcacgccatt
ctgcggcggcaggaagatttttacccattcctgaaggacaaccgggaaaagatcgagaagatcctgaccttccgcatcccctactacgtgg
gccctctggccaggggaaacagcagattcgcctggatgaccagaaagagcgaggaaaccatcaccccctggaacttcgaggaagtggt
ggacaagggcgcttccgcccagagcttcatcgagcggatgaccaacttcgataagaacctgcccaacgagaaggtgctgcccaagcac
agcctgctgtacgagtacttcaccgtgtataacgagctgaccaaagtgaaatacgtgaccgagggaatgagaaagcccgccttcctgagc
ggcgagcagaaaaaggccatcgtggacctgctgttcaagaccaaccggaaagtgaccgtgaagcagctgaaagaggactacttcaaga
aaatcgagtgcttcgactccgtggaaatctccggcgtggaagatcggttcaacgcctccctgggcacataccacgatctgctgaaaattatc
aaggacaaggacttcctggacaatgaggaaaacgaggacattctggaagatatcgtgctgaccctgacactgtttgaggacagagagatg
atcgaggaacggctgaaaacctatgcccacctgttcgacgacaaagtgatgaagcagctgaagcggcggagatacaccggctggggca
ggctgagccggaagctgatcaacggcatccgggacaagcagtccggcaagacaatcctggatttcctgaagtccgacggcttcgccaac
agaaacttcatgcagctgatccacgacgacagcctgacctttaaagaggacatccagaaagcccaggtgtccggccagggcgatagcct
gcacgagcacattgccaatctggccggcagccccgccattaagaagggcatcctgcagacagtgaaggtggtggacgagctcgtgaaa
gtgatgggccggcacaagcccgagaacatcgtgatcgaaatggccagagagaaccagaccacccagaagggacagaagaacagccg
cgagagaatgaagcggatcgaagagggcatcaaagagctgggcagccagatcctgaaagaacaccccgtggaaaacacccagctgca
gaacgagaagctgtacctgtactacctgcagaatggggggatatgtacgtggaccaggaactggacatcaaccggctgtccgactacga
tgtggacgctatcgtgcctcagagctttctgaaggacgactccatcgacaacaaggtgctgaccagaagcgacaagaaccggggcaaga
gcgacaacgtgccctccgaagaggtcgtgaagaagatgaagaactactggcggcagctgctgaacgccaagctgattacccagagaaa
gttcgacaatctgaccaaggccgagagaggcggcctgagcgaactggataaggccggcttcatcaagagacagctggtggaaacccgg
cagatcacaaagcacgtggcacagatcctggactcccggatgaacactaagtacgacgagaatgacaagctgatccgggaagtgaaagt
gatcaccctgaagtccaagctggtgtccgatttccggaaggatttccagttttacaaagtgcgcgagatcaacaactaccaccacgcccacg
acgcctacctgaacgccgtcgtgggaaccgccctgatcaaaaagtaccctaagctggaaagcgagttcgtgtacggcgactacaaggtgt
acgacgtgcggaagatgatcgccaagagcgagcaggaaatcggcaaggctaccgccaagtacttcttctacagcaacatcatgaacttttt
caagaccgagattaccctggccaacggcgagatccggaagcggcctctgatcgagacaaacggcgaaaccggggagatcgtgtggga
taagggccgggattttgccaccgtgcggaaagtgctgagcatgccccaagtgaatatcgtgaaaaagaccgaggtgcagacaggcggct
tcagcaaagagtctatcctgcccaagaggaacagcgataagctgatcgccagaaagaaggactgggaccctaagaagtacggcggcttc
gacagccccaccgtggcctattctgtgctggtggtggccaaagtggaaaagggcaagtccaagaaactgaagagtgtgaaagagctgct
ggggatcaccatcatggaaagaagcagcttcgagaagaatcccatcgactttctggaagccaagggctacaaagaagtgaaaaaggacc
tgatcatcaagctgcctaagtactccctgttcgagctggaaaacggccggaagagaatgctggcctctgccggcgaactgcagaagggaa
acgaactggccctgccctccaaatatgtgaacttcctgtacctggccagccactatgagaagctgaagggctcccccgaggataatgagca
gaaacagctgtttgtggaacagcacaagcactacctggacgagatcatcgagcagatcagcgagttctccaagagagtgatcctggccga
cgctaatctggacaaagtgctgtccgcctacaacaagcaccgggataagcccatcagagagcaggccgagaatatcatccacctgtttac
cctgaccaatctgggagcccctgccgccttcaagtactttgacaccaccatcgaccggaagaggtacaccagcaccaaagaggtgctgg
acgccaccctgatccaccagagcatcaccggcctgtacgagacacggatcgacctgtctcagctgggaggcgacaaaaggccggcgg
ccacgaaaaaggccggacaggccaaaaagaaaaagctcgagggcggaggcgggagcggatccccctcccggctccagatgttcttcg
ctaataaccacgaccaggaatttgaccctccaaaggtttacccacctgtcccagctgagaagaggaagcccatccgggtgctgtctctcttt
gatggaatcgctacagggctcctggtgctgaaggacttgggcattcaggtggaccgctacattgcctcggaggtgtgtgaggactccatca
cggtgggcatggtgcggcaccaggggaagatcatgtacgtcggggacgtccgcagcgtcacacagaagcatatccaggagtggggcc
cattcgatctggtgattgggggcagtccctgcaatgacctctccatcgtcaaccctgctcgcaagggcctctacgagggcactggccggct
cttctttgagttctaccgcctcctgcatgatgcgcggcccaaggagggagatgatcgccccttcttctggctctttgcgaatgtggtggccatg
ggcgttagtgacaagagggacatctcgcgatttctcgagtccaaccctgtgatgattgatgccaaagaagtgtcagctgcacacagggccc
gctacttctggggtaaccttcccggtatgaacaggccgttggcatccactgtgaatgataagctggagctgcaggagtgtctggagcatgg
caggatagccaagttcagcaaagtgaggaccattactacgaggtcaaactccataaagcagggcaaagaccagcattttcctgtgttcatg
aatgagaaagaggacatcttatggtgcactgaaatggaaagggtatttggtttcccagtccactatactgacgtgtccaacatgagccgcttg
gcgaggcagagactgctgggccggtcatggagcgtgccagtcatccgccacctcttcgctccgctgaaggagtattttgcgtgtgtgtccg
gccggcccggatccggcgcaacaaacttctctctgctgaaacaagccggagatgtcgaagagaatcctggaccgaccgagtacaagcc
cacggtgcgcctcgccacccgcgacgacgtccccagggccgtacgcaccctcgccgccgcgttcgccgactaccccgccacgcgcca
caccgtcgatccggaccgccacatcgagcgggtcaccgagctgcaagaactcttcctcacgcgcgtcgggctcgacatcggcaaggtgt
gggtcgcggacgacggcgccgcggtggcggtctggaccacgccggagagcgtcgaagcgggggcggtgttcgccgagatcggccc
gcgcatggccgagttgagcggttcccggctggccgcgcagcaacagatggaaggcctcctggcgccgcaccggcccaaggagcccg
cgtggttcctggccaccgtcggagtctcgcccgaccaccagggcaagggtctgggcagcgccgtcgtgctccccggagtggaggcgg
ccgagcgcgccggggtgcccgccttcctggagacctccgcgccccgcaacctccccttctacgageggcteggcttcaccgtcaccgcc
gacgtcgaggtgcccgaaggaccgcgcacctggtgcatgacccgcaagcccggtgcctgaacgcgttaagtcgacaatcaacctctggatt
acaaaatttgtgaaagattgactggtattcttaactatgttgctccttttacgctatgtggatacgctgctttaatgcctttgtatcatgctattgc
ttcccgtatggctttcattttctcctccttgtataaatcctggttgctgtctctttatgaggagttgtggcccgttgtcaggcaacgtggcgtggtgt
gcactgtgtttgctgacgcaacccccactggttggggcattgccaccacctgtcagctcctttccgggactttcgctttccccctccctattgcc
acggcggaactcatcgccgcctgccttgcccgctgctggacaggggctcggctgttgggcactgacaattccgtggtgttgtcggggaaa
tcatcgtcctttccttggctgctcgcctgtgttgccacctggattctgcgcgggacgtccttctgctacgtcccttcggccctcaatccagcgg
accttccttcccgcggcctgctgccggctctgcggcctcttccgcgtcttcgccttcgccctcagacgagtcggatctccctttgggccgcct
ccccgcgtcgactttaagaccaatgacttacaaggcagctgtagatcttagccactttttaaaagaaaaggggggactggaagggctaattc
actcccaacgaagacaagatctgctttttgcttgtactgggtctctctggttagaccagatctgagcctgggagctctctggctaactagggaa
cccactgcttaagcctcaataaagcttgccttgagtgcttcaagtagtgtgtgcccgtctgttgtgtgactctggtaactagagatccctcagac
ccttttagtcagtgtggaaaatctctagcagggcccgtttaaacccgctgatcagcctcgactgtgccttctagttgccagccatctgttgtttg
cccctcccccgtgccttccttgaccctggaaggtgccactcccactgtcctttcctaataaaatgaggaaattgcatcgcattgtctgagtagg
tgtcattctattctggggggtggggggggcaggacagcaagggggaggattgggaagacaatagcaggcatgctggggatgcggtgg
gctctatggcttctgaggcggaaagaaccagctggggctctagggggtatccccacgcgccctgtagcggcgcattaagcgcggcgggt
gtggtggttacgcgcagcgtgaccgctacacttgccagcgccctagcgcccgctcctttcgctttcttcccttcctttctegccacgttcgccg
gctttccccgtcaagctctaaatcgggggctccctttagggttccgatttagtgctttacggcacctcgaccccaaaaaacttgattagggtga
tggttcacgtagtgggccatcgccctgatagacggtttttcgccctttgacgttggagtccacgttctttaatagtggactcttgttccaaactgg
aacaacactcaaccctatctcggtctattcttttgatttataagggattttgccgatttcggcctattggttaaaaaatgagctgatttaacaaaaat
ttaacgcgaattaattctgtggaatgtgtgtcagttagggtgtggaaagtccccaggctccccagcaggcagaagtatgcaaagcatgcatc
tcaattagtcagcaaccaggtgtggaaagtccccaggctccccagcaggcagaagtatgcaaagcatgcatctcaattagtcagcaaccat
agtcccgcccctaactccgcccatcccgcccctaactccgcccagttccgcccattctccgccccatggctgactaattttttttatttatgcag
aggccgaggccgcctctgcctctgagctattccagaagtagtgaggaggcttttttggaggcctaggcttttgcaaaaagctcccgggagc
ttgtatatccattttcggatctgatcagcacgtgttgacaattaatcatcggcatagtatatcggcatagtataatacgacaaggtgaggaacta
aaccatggccaagttgaccagtgccgttccggtgctcaccgcgcgcgacgtcgccggagcggtcgagttctggaccgaccggctcgggt
tctcccgggacttcgtggaggacgacttcgccggtgtggtccgggacgacgtgaccctgttcatcagcgcggtccaggaccaggtggtg
ccggacaacaccctggcctgggtgtgggtgcgcggcctggacgagctgtacgccgagtggtcggaggtcgtgtccacgaacttccggg
acgcctccgggccggccatgaccgagatcggcgagcagccgtggggggggagttcgccctgcgcgacccggccggcaactgcgtg
cacttcgtggccgaggagcaggactgacacgtgctacgagatttcgattccaccgccgccttctatgaaaggttgggcttcggaatcgttttc
cgggacgccggctggatgatcctccagcgcggggatctcatgctggagttcttcgcccaccccaacttgtttattgcagcttataatggttacaaa
taaagcaatagcatcacaaatttcacaaataaagcatttttttcactgcattctagttgtggtttgtccaaactcatcaatgtatcttatcatgtc
tgtataccgtcgacctctagctagagcttggcgtaatcatggtcatagctgtttcctgtgtgaaattgttatccgctcacaattccacacaacata
cgagccggaagcataaagtgtaaagcctggggtgcctaatgagtgagctaactcacattaattgcgttgcgctcactgcccgctttccagtc
gggaaacctgtcgtgccagctgcattaatgaatcggccaacgcgcggggagaggcggtttgcgtattgggcgctcttccgcttcctcgctc
actgactcgctgcgctcggtcgttcggctgcggcgagcggtatcagctcactcaaaggcggtaatacggttatccacagaatcaggggata
acgcaggaaagaacatgtgagcaaaaggccagcaaaaggccaggaaccgtaaaaaggccgcgttgctggcgtttttccataggctccgc
ccccctgacgagcatcacaaaaatcgacgctcaagtcagaggtggcgaaacccgacaggactataaagataccaggcgtttccccctgg
aagctccctcgtgcgctctcctgttccgaccctgccgcttaccggatacctgtccgcctttctcccttcgggaagcgtggcgctttctcatagc
tcacgctgtaggtatctcagttcggtgtaggtcgttcgctccaagctgggctgtgtgcacgaaccccccgttcagcccgaccgctgcgcctt
atccggtaactatcgtcttgagtccaacccggtaagacacgacttatcgccactggcagcagccactggtaacaggattagcagagcgag
gtatgtaggcggtgctacagagttcttgaagtggtggcctaactacggctacactagaagaacagtatttggtatctgcgctctgctgaagcc
agttaccttcggaaaaagagttggtagctcttgatccggcaaacaaaccaccgctggtagcggtggtttttttgtttgcaagcagcagattac
gcgcagaaaaaaaggatctcaagaagatcctttgatcttttctacggggtctgacgctcagtggaacgaaaactcacgttaagggattttggt
catgagattatcaaaaaggatcttcacctagatccttttaaattaaaaatgaagttttaaatcaatctaaagtatatatgagtaaacttggtctgac
agttaccaatgcttaatcagtgaggcacctatctcagcgatctgtctatttcgttcatccatagttgcctgactccccgtcgtgtagataactacg
atacgggagggcttaccatctggccccagtgctgcaatgataccgcgagacccacgctcaccggctccagatttatcagcaataaaccag
ccagccggaagggccgagcgcagaagtggtcctgcaactttatccgcctccatccagtctattaattgttgccgggaagctagagtaagta
gttcgccagttaatagtttgcgcaacgttgttgccattgctacaggcatcgtggtgtcacgctcgtcgtttggtatggcttcattcagctccggtt
cccaacgatcaaggcgagttacatgatcccccatgttgtgcaaaaaagcggttagctccttcggtcctccgatcgttgtcagaagtaagttgg
ccgcagtgttatcactcatggttatggcagcactgcataattctcttactgtcatgccatccgtaagatgcttttctgtgactggtgagtactcaa
ccaagtcattctgagaatagtgtatgcggcgaccgagttgctcttgcccggcgtcaatacgggataataccgcgccacatagcagaacttta
aaagtgctcatcattggaaaacgttcttcggggcgaaaactctcaaggatcttaccgctgttgagatccagttcgatgtaacccactcgtgca
cccaactgatcttcagcatcttttactttcaccagcgtttctgggtgagcaaaaacaggaaggcaaaatgccgcaaaaaagggaataaggg
cgacacggaaatgttgaatactcatactcttcctttttcaatattattgaagcatttatcagggttattgtctcatgagcggatacatatttgaat
gtatttagaaaaataaacaaataggggttccgcgcacatttccccgaaaagtgccacctgac.

In an aspect, a disclosed pBK1031 plasmid can comprise the sequence set forth below or a sequence having 70%, 75%, 80%, 85%, 90%, 95%, or more identity to the sequence set forth

(SEQ ID NO: 34)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggtggagtatttacggtaaactgcccacttggcagtacatcaag
tgtatcatatgccaagtacgccccctattgacgtcaatgacggtaaat
ggcccgcctggcattatgcccagtacatgaccttatgggactttccta
cttggcagtacatctacgtattagtcatcgctattaccatggtgatgc
ggttttggcagtacatcaatgggcgtggatagcggtttgactcacggg
gatttccaagtctccaccccattgacgtcaatgggagtttgttttggc
accaaaatcaacgggactttccaaaatgtcgtaacaactccgccccat
tgacgcaaatgggcggtaggcgtgtacggtgggaggtctatataagca
gcgcgttttgcctgtactgggtctctctggttagaccagatctgagcc
tgggagctctctggctaactagggaacccactgcttaagcctcaataa
agcttgccttgagtgcttcaagtagtgtgtgcccgtctgttgtgtgac
tctggtaactagagatccctcagacccttttagtcagtgtggaaaatc
tctagcagtggcgcccgaacagggacttgaaagcgaaagggaaaccag
aggagctctctcgacgcaggactcggcttgctgaagcgcgcacggcaa
gaggcgaggggcggcgactggtgagtacgccaaaaattttgactagcg
gaggctagaaggagagagatgggtgcgagagcgtcagtattaagcggg
ggagaattagatcgcgatgggaaaaaattcggttaaggccagggggaa
agaaaaaatataaattaaaacatatagtatgggcaagcagggagctag
aacgattcgcagttaatcctggcctgttagaaacatcagaaggctgta
gacaaatactgggacagctacaaccatcccttcagacaggatcagaag
aacttagatcattatataatacagtagcaaccctctattgtgtgcatc
aaaggatagagataaaagacaccaaggaagctttagacaagatagagg
aagagcaaaacaaaagtaagaccaccgcacagcaagcggccgctgatc
ttcagacctggaggaggagatatgagggacaattggagaagtgaatta
tataaatataaagtagtaaaaattgaaccattaggagtagcacccacc
aaggcaaagagaagagtggtgcagagagaaaaaagagcagtgggaata
ggagctttgttccttgggttcttgggagcagcaggaagcactatgggc
gcagcgtcaatgacgctgacggtacaggccagacaattattgtctggt
atagtgcagcagcagaacaatttgctgagggctattgaggcgcaacag
catctgttgcaactcacagtctggggcatcaagcagctccaggcaaga
atcctggctgtggaaagatacctaaaggatcaacagctcctggggatt
tggggttgctctggaaaactcatttgcaccactgctgtgccttggaat
gctagttggagtaataaatctctggaacagatttggaatcacacgacc
tggatggagtgggacagagaaattaacaattacacaagcttaatacac
tccttaattgaagaatcgcaaaaccagcaagaaaagaatgaacaagaa
ttattggaattagataaatgggcaagtttgtggaattggtttaacata
acaaattggctgtggtatataaaattattcataatgatagtaggaggc
ttggtaggtttaagaatagtttttgctgtactttctatagtgaataga
gttaggcagggatattcaccattatcgtttcagacccacctcccaacc
ccgaggggacccgacaggcccgaaggaatagaagaagaaggtggagag
agagacagagacagatccattcgattagtgaacggatcggcactgcgt
gcgccaattctgcagacaaatggcagtattcatccacaattttaaaag
aaaaggggggattggggggtacagtgcaggggaaagaatagtagacat
aatagcaacagacatacaaactaaagaattacaaaaacaaattacaaa
aattcaaaattttcgggtttattacagggacagcagagatccagtttg
gttaattaatggggggacgttaacggggcggaacggtaccgagggcct
atttcccatgattccttcatatttgcatatacgatacaaggctgttag
agagataattagaattaatttgactgtaaacacaaagatattagtaca
aaatacgtgacgtagaaagtaataatttcttgggtagtttgcagtttt
aaaattatgttttaaaatggactatcatatgcttaccgtaacttgaaa
gtatttcgatttcttggctttatatatcttgtggaaaggacgaaacac
cgtcaggagagctactcggggtgttttagagctagaaatagcaagtta
aaataaggctagtccgttatcaacttgaaaaagtggcaccgagtcggt
gcttttttgaattcgctagctaggtcttgaaaggagtgggaattggct
ccggtgcccgtcagtgggcagagcgcacatcgcccacagtccccgaga
agttggggggaggggtcggcaattgatccggtgcctagagaaggtggc
gcggggtaaactgggaaagtgatgtcgtgtactggctccgcctttttc
ccgaggggggggagaaccgtatataagtgcagtagtcgccgtgaacgt
tctttttcgcaacgggtttgccgccagaacacaggaccggtgccacca
tggactataaggaccacgacggagactacaaggatcatgatattgatt
acaaagacgatgacgataagatggccccaaagaagaagcggaaggtcg
gtatccacggagtcccagcagccgacaagaagtacagcatcggcctgg
ccatcggcaccaactctgtgggctgggccgtgatcaccgacgagtaca
aggtgcccagcaagaaattcaaggtgctgggcaacaccgaccggcaca
gcatcaagaagaacctgatcggagccctgctgttcgacagcggcgaaa
cagccgaggccacccggctgaagagaaccgccagaagaagatacacca
gacggaagaaccggatctgctatctgcaagagatcttcagcaacgaga
tggccaaggtggacgacagcttcttccacagactggaagagtccttcc
tggtggaagaggataagaagcacgagcggcaccccatcttcggcaaca
tcgtggacgaggtggcctaccacgagaagtaccccaccatctaccacc
tgagaaagaaactggtggacagcaccgacaaggccgacctgcggctga
tctatctggccctggcccacatgatcaagttccggggccacttcctga
tcgagggcgacctgaaccccgacaacagcgacgtggacaagctgttca
tccagctggtgcagacctacaaccagctgttcgaggaaaaccccatca
acgccagcggcgtggacgccaaggccatcctgtctgccagactgagca
agagcagacggctggaaaatctgatcgcccagctgcccggcgagaaga
agaatggcctgttcggcaacctgattgccctgagcctgggcctgaccc
ccaacttcaagagcaacttcgacctggccgaggatgccaaactgcagc
tgagcaaggacacctacgacgacgacctggacaacctgctggcccaga
tcggcgaccagtacgccgacctgtttctggccgccaagaacctgtccg
acgccatcctgctgagcgacatcctgagagtgaacaccgagatcacca
aggcccccctgagcgcctctatgatcaagagatacgacgagcaccacc
aggacctgaccctgctgaaagctctcgtgcggcagcagctgcctgaga
agtacaaagagattttcttcgaccagagcaagaacggctacgccggct
acattgacggcggagccagccaggaagagttctacaagttcatcaagc
ccatcctggaaaagatggacggcaccgaggaactgctcgtgaagctga
acagagaggacctgctgcggaagcagcggaccttcgacaacggcagca
tcccccaccagatccacctgggagagctgcacgccattctgcggcggc
aggaagatttttacccattcctgaaggacaaccgggaaaagatcgaga
agatcctgaccttccgcatcccctactacgtgggccctctggccaggg
gaaacagcagattcgcctggatgaccagaaagagcgaggaaaccatca
ccccctggaacttcgaggaagtggtggacaagggcgcttccgcccaga
gcttcatcgagcggatgaccaacttcgataagaacctgcccaacgaga
aggtgctgcccaagcacagcctgctgtacgagtacttcaccgtgtata
acgagctgaccaaagtgaaatacgtgaccgagggaatgagaaagcccg
ccttcctgagcggcgagcagaaaaaggccatcgtggacctgctgttca
agaccaaccggaaagtgaccgtgaagcagctgaaagaggactacttca
agaaaatcgagtgcttcgactccgtggaaatctccggcgtggaagatc
ggttcaacgcctccctgggcacataccacgatctgctgaaaattatca
aggacaaggacttcctggacaatgaggaaaacgaggacattctggaag
atatcgtgctgaccctgacactgtttgaggacagagagatgatcgagg
aacggctgaaaacctatgcccacctgttcgacgacaaagtgatgaagc
agctgaagcggcggagatacaccggctggggcaggctgagccggaagc
tgatcaacggcatccgggacaagcagtccggcaagacaatcctggatt
tcctgaagtccgacggcttcgccaacagaaacttcatgcagctgatcc
acgacgacagcctgacctttaaagaggacatccagaaagcccaggtgt
ccggccagggcgatagcctgcacgagcacattgccaatctggccggca
gccccgccattaagaagggcatcctgcagacagtgaaggtggtggacg
agctcgtgaaagtgatgggccggcacaagcccgagaacatcgtgatcg
aaatggccagagagaaccagaccacccagaagggacagaagaacagcc
gcgagagaatgaagcggatcgaagagggcatcaaagagctgggcagcc
agatcctgaaagaacaccccgtggaaaacacccagctgcagaacgaga
agctgtacctgtactacctgcagaatggggggatatgtacgtggacca
ggaactggacatcaaccggctgtccgactacgatgtggacgctatcgt
gcctcagagctttctgaaggacgactccatcgacaacaaggtgctgac
cagaagcgacaagaaccggggcaagagcgacaacgtgccctccgaaga
ggtcgtgaagaagatgaagaactactggcggcagctgctgaacgccaa
gctgattacccagagaaagttcgacaatctgaccaaggccgagagagg
cggcctgagcgaactggataaggccggcttcatcaagagacagctggt
ggaaacccggcagatcacaaagcacgtggcacagatcctggactcccg
gatgaacactaagtacgacgagaatgacaagctgatccgggaagtgaa
agtgatcaccctgaagtccaagctggtgtccgatttccggaaggattt
ccagttttacaaagtgcgcgagatcaacaactaccaccacgcccacga
cgcctacctgaacgccgtcgtgggaaccgccctgatcaaaaagtaccc
taagctggaaagcgagttcgtgtacggcgactacaaggtgtacgacgt
gcggaagatgatcgccaagagcgagcaggaaatcggcaaggctaccgc
caagtacttcttctacagcaacatcatgaactttttcaagaccgagat
taccctggccaacggcgagatccggaagcggcctctgatcgagacaaa
cggcgaaaccggggagatcgtgtgggataagggccgggattttgccac
cgtgcggaaagtgctgagcatgccccaagtgaatatcgtgaaaaagac
cgaggtgcagacaggcggcttcagcaaagagtctatcctgcccaagag
gaacagcgataagctgatcgccagaaagaaggactgggaccctaagaa
gtacggcggcttcgacagccccaccgtggcctattctgtgctggtggt
ggccaaagtggaaaagggcaagtccaagaaactgaagagtgtgaaaga
gctgctggggatcaccatcatggaaagaagcagcttcgagaagaatcc
catcgactttctggaagccaagggctacaaagaagtgaaaaaggacct
gatcatcaagctgcctaagtactccctgttcgagctggaaaacggccg
gaagagaatgctggcctctgccggcgaactgcagaagggaaacgaact
ggccctgccctccaaatatgtgaacttcctgtacctggccagccacta
tgagaagctgaagggctcccccgaggataatgagcagaaacagctgtt
tgtggaacagcacaagcactacctggacgagatcatcgagcagatcag
cgagttctccaagagagtgatcctggccgacgctaatctggacaaagt
gctgtccgcctacaacaagcaccgggataagcccatcagagagcaggc
cgagaatatcatccacctgtttaccctgaccaatctgggagcccctgc
cgccttcaagtactttgacaccaccatcgaccggaagaggtacaccag
caccaaagaggtgctggacgccaccctgatccaccagagcatcaccgg
cctgtacgagacacggatcgacctgtctcagctgggaggcgacaaaag
gccggcggccacgaaaaaggccggacaggccaaaaagaaaaagctcga
gggcggaggcgggagcggatccccctcccggctccagatgttcttcgc
taataaccacgaccaggaatttgaccctccaaaggtttacccacctgt
cccagctgagaagaggaagcccatccgggtgctgtctctctttgatgg
aatcgctacagggctcctggtgctgaaggacttgggcattcaggtgga
ccgctacattgcctcggaggtgtgtgaggactccatcacggtgggcat
ggtgcggcaccaggggaagatcatgtacgtcggggacgtccgcagcgt
cacacagaagcatatccaggagtggggcccattcgatctggtgattgg
gggcagtccctgcaatgacctctccatcgtcaaccctgctcgcaaggg
cctctacgagggcactggccggctcttctttgagttctaccgcctcct
gcatgatgcgcggcccaaggagggagatgatcgccccttcttctggct
ctttgcgaatgtggtggccatgggcgttagtgacaagagggacatctc
gcgatttctcgagtccaaccctgtgatgattgatgccaaagaagtgtc
agctgcacacagggcccgctacttctggggtaaccttcccggtatgaa
caggccgttggcatccactgtgaatgataagctggagctgcaggagtg
tctggagcatggcaggatagccaagttcagcaaagtgaggaccattac
tacgaggtcaaactccataaagcagggcaaagaccagcattttcctgt
gttcatgaatgagaaagaggacatcttatggtgcactgaaatggaaag
ggtatttggtttcccagtccactatactgacgtgtccaacatgagccg
cttggcgaggcagagactgctgggccggtcatggagcgtgccagtcat
ccgccacctcttcgctccgctgaaggagtattttgcgtgtgtgtccgg
ccggcccggatccggcgcaacaaacttctctctgctgaaacaagccgg
agatgtcgaagagaatcctggaccgaccgagtacaagcccacggtgcg
cctcgccacccgcgacgacgtccccagggccgtacgcaccctcgccgc
cgcgttcgccgactaccccgccacgcgccacaccgtcgatccggaccg
ccacatcgagcgggtcaccgagctgcaagaactcttcctcacgcgcgt
cgggctcgacatcggcaaggtgtgggtcgcggacgacggcgccgcggt
ggcggtctggaccacgccggagagcgtcgaagcgggggcggtgttcgc
cgagatcggcccgcgcatggccgagttgagcggttcccggctggccgc
gcagcaacagatggaaggcctcctggcgccgcaccggcccaaggagcc
cgcgtggttcctggccaccgtcggagtctcgcccgaccaccagggcaa
gggtctgggcagcgccgtcgtgctccccggagtggaggcggccgagcg
cgccggggtgcccgccttcctggagacctccgcgccccgcaacctccc
cttctacgagcggctcggcttcaccgtcaccgccgacgtcgaggtgcc
cgaaggaccgcgcacctggtgcatgacccgcaagcccggtgcctgaac
gcgttaagtcgacaatcaacctctggattacaaaatttgtgaaagatt
gactggtattcttaactatgttgctccttttacgctatgtggatacgc
tgctttaatgcctttgtatcatgctattgcttcccgtatggctttcat
tttctcctccttgtataaatcctggttgctgtctctttatgaggagtt
gtggcccgttgtcaggcaacgtggcgtggtgtgcactgtgtttgctga
cgcaacccccactggttggggcattgccaccacctgtcagctcctttc
cgggactttcgctttccccctccctattgccacggcggaactcatcgc
cgcctgccttgcccgctgctggacaggggctcggctgttgggcactga
caattccgtggtgttgtcggggaaatcatcgtcctttccttggctgct
cgcctgtgttgccacctggattctgcgcgggacgtccttctgctacgt
cccttcggccctcaatccagcggaccttccttcccgcggcctgctgcc
ggctctgcggcctcttccgcgtcttcgccttcgccctcagacgagtcg
gatctccctttgggccgcctccccgcgtcgactttaagaccaatgact
tacaaggcagctgtagatcttagccactttttaaaagaaaagggggga
ctggaagggctaattcactcccaacgaagacaagatctgctttttgct
tgtactgggtctctctggttagaccagatctgagcctgggagctctct
ggctaactagggaacccactgcttaagcctcaataaagcttgccttga
gtgcttcaagtagtgtgtgcccgtctgttgtgtgactctggtaactag
agatccctcagacccttttagtcagtgtggaaaatctctagcagggcc
cgtttaaacccgctgatcagcctcgactgtgccttctagttgccagcc
atctgttgtttgcccctcccccgtgccttccttgaccctggaaggtgc
cactcccactgtcctttcctaataaaatgaggaaattgcatcgcattg
tctgagtaggtgtcattctattctggggggtggggggggcaggacagc
aagggggaggattgggaagacaatagcaggcatgctggggatgcggtg
ggctctatggcttctgaggcggaaagaaccagctggggctctaggggg
tatccccacgcgccctgtagcggcgcattaagcgcggcgggtgtggtg
gttacgcgcagcgtgaccgctacacttgccagcgccctagcgcccgct
cctttcgctttcttcccttcctttctcgccacgttcgccggctttccc
cgtcaagctctaaatcgggggctccctttagggttccgatttagtgct
ttacggcacctcgaccccaaaaaacttgattagggtgatggttcacgt
agtgggccatcgccctgatagacggtttttcgccctttgacgttggag
tccacgttctttaatagtggactcttgttccaaactggaacaacactc
aaccctatctcggtctattcttttgatttataagggattttgccgatt
tcggcctattggttaaaaaatgagctgatttaacaaaaatttaacgcg
aattaattctgtggaatgtgtgtcagttagggtgtggaaagtccccag
gctccccagcaggcagaagtatgcaaagcatgcatctcaattagtcag
caaccaggtgtggaaagtccccaggctccccagcaggcagaagtatgc
aaagcatgcatctcaattagtcagcaaccatagtcccgcccctaactc
cgcccatcccgcccctaactccgcccagttccgcccattctccgcccc
atggctgactaattttttttatttatgcagaggccgaggccgcctctg
cctctgagctattccagaagtagtgaggaggcttttttggaggcctag
gcttttgcaaaaagctcccgggagcttgtatatccattttcggatctg
atcagcacgtgttgacaattaatcatcggcatagtatatcggcatagt
ataatacgacaaggtgaggaactaaaccatggccaagttgaccagtgc
cgttccggtgctcaccgcgcgcgacgtcgccggagcggtcgagttctg
gaccgaccggctcgggttctcccgggacttcgtggaggacgacttcgc
cggtgtggtccgggacgacgtgaccctgttcatcagcgcggtccagga
ccaggtggtgccggacaacaccctggcctgggtgtgggtgcgcggcct
ggacgagctgtacgccgagtggtcggaggtcgtgtccacgaacttccg
ggacgcctccgggccggccatgaccgagatcggcgagcagccgtgggg
ggggagttcgccctgcgcgacccggccggcaactgcgtgcacttcgtg
gccgaggagcaggactgacacgtgctacgagatttcgattccaccgcc
gccttctatgaaaggttgggcttcggaatcgttttccgggacgccggc
tggatgatcctccagcgcggggatctcatgctggagttcttcgcccac
cccaacttgtttattgcagcttataatggttacaaataaagcaatagc
atcacaaatttcacaaataaagcatttttttcactgcattctagttgt
ggtttgtccaaactcatcaatgtatcttatcatgtctgtataccgtcg
acctctagctagagcttggcgtaatcatggtcatagctgtttcctgtg
tgaaattgttatccgctcacaattccacacaacatacgagccggaagc
ataaagtgtaaagcctggggtgcctaatgagtgagctaactcacatta
attgcgttgcgctcactgcccgctttccagtcgggaaacctgtcgtgc
cagctgcattaatgaatcggccaacgcgcggggagaggcggtttgcgt
attgggcgctcttccgcttcctcgctcactgactcgctgcgctcggtc
gttcggctgcggcgagcggtatcagctcactcaaaggcggtaatacgg
ttatccacagaatcaggggataacgcaggaaagaacatgtgagcaaaa
ggccagcaaaaggccaggaaccgtaaaaaggccgcgttgctggcgttt
ttccataggctccgcccccctgacgagcatcacaaaaatcgacgctca
agtcagaggtggcgaaacccgacaggactataaagataccaggcgttt
ccccctggaagctccctcgtgcgctctcctgttccgaccctgccgctt
accggatacctgtccgcctttctcccttcgggaagcgtggcgctttct
catagctcacgctgtaggtatctcagttcggtgtaggtcgttcgctcc
aagctgggctgtgtgcacgaaccccccgttcagcccgaccgctgcgcc
ttatccggtaactatcgtcttgagtccaacccggtaagacacgactta
tcgccactggcagcagccactggtaacaggattagcagagcgaggtat
gtaggcggtgctacagagttcttgaagtggtggcctaactacggctac
actagaagaacagtatttggtatctgcgctctgctgaagccagttacc
ttcggaaaaagagttggtagctcttgatccggcaaacaaaccaccgct
ggtagcggtggtttttttgtttgcaagcagcagattacgcgcagaaaa
aaaggatctcaagaagatcctttgatcttttctacggggtctgacgct
cagtggaacgaaaactcacgttaagggattttggtcatgagattatca
aaaaggatcttcacctagatccttttaaattaaaaatgaagttttaaa
tcaatctaaagtatatatgagtaaacttggtctgacagttaccaatgc
ttaatcagtgaggcacctatctcagcgatctgtctatttcgttcatcc
atagttgcctgactccccgtcgtgtagataactacgatacgggagggc
ttaccatctggccccagtgctgcaatgataccgcgagacccacgctca
ccggctccagatttatcagcaataaaccagccagccggaagggccgag
cgcagaagtggtcctgcaactttatccgcctccatccagtctattaat
tgttgccgggaagctagagtaagtagttcgccagttaatagtttgcgc
aacgttgttgccattgctacaggcatcgtggtgtcacgctcgtcgttt
ggtatggcttcattcagctccggttcccaacgatcaaggcgagttaca
tgatcccccatgttgtgcaaaaaagcggttagctccttcggtcctccg
atcgttgtcagaagtaagttggccgcagtgttatcactcatggttatg
gcagcactgcataattctcttactgtcatgccatccgtaagatgcttt
tctgtgactggtgagtactcaaccaagtcattctgagaatagtgtatg
cggcgaccgagttgctcttgcccggcgtcaatacgggataataccgcg
ccacatagcagaactttaaaagtgctcatcattggaaaacgttcttcg
gggcgaaaactctcaaggatcttaccgctgttgagatccagttcgatg
taacccactcgtgcacccaactgatcttcagcatcttttactttcacc
agcgtttctgggtgagcaaaaacaggaaggcaaaatgccgcaaaaaag
ggaataagggcgacacggaaatgttgaatactcatactcttccttttt
caatattattgaagcatttatcagggttattgtctcatgagcggatac
atatttgaatgtatttagaaaaataaacaaataggggttccgcgcaca
tttccccgaaaagtgccacctgac.

In an aspect, a disclosed pBK1032 plasmid can comprise the sequence set forth below or a sequence having 70%, 75%, 80%, 85%, 90%, 95%, or more identity to the sequence set forth

(SEQ ID NO: 35)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggtggagtatttacggtaaactgcccacttggcagtacatcaag
tgtatcatatgccaagtacgccccctattgacgtcaatgacggtaaat
ggcccgcctggcattatgcccagtacatgaccttatgggactttccta
cttggcagtacatctacgtattagtcatcgctattaccatggtgatgc
ggttttggcagtacatcaatgggcgtggatagcggtttgactcacggg
gatttccaagtctccaccccattgacgtcaatgggagtttgttttggc
accaaaatcaacgggactttccaaaatgtcgtaacaactccgccccat
tgacgcaaatgggggtaggcgtgtacggtgggaggtctatataagcag
cgcgttttgcctgtactgggtctctctggttagaccagatctgagcct
gggagctctctggctaactagggaacccactgcttaagcctcaataaa
gcttgccttgagtgcttcaagtagtgtgtgcccgtctgttgtgtgact
ctggtaactagagatccctcagacccttttagtcagtgtggaaaatct
ctagcagtggcgcccgaacagggacttgaaagcgaaagggaaaccaga
ggagctctctcgacgcaggactcggcttgctgaagcgcgcacggcaag
aggcgaggggcggcgactggtgagtacgccaaaaattttgactagcgg
aggctagaaggagagagatgggtgcgagagcgtcagtattaagcgggg
gagaattagatcgcgatgggaaaaaattcggttaaggccagggggaaa
gaaaaaatataaattaaaacatatagtatgggcaagcagggagctaga
acgattcgcagttaatcctggcctgttagaaacatcagaaggctgtag
acaaatactgggacagctacaaccatcccttcagacaggatcagaaga
acttagatcattatataatacagtagcaaccctctattgtgtgcatca
aaggatagagataaaagacaccaaggaagctttagacaagatagagga
agagcaaaaaaaagtaagaccaccgcacagcaagcggccgctgatctt
cagacctggaggaggagatatgagggacaattggagaagtgaattata
taaatataaagtagtaaaaattgaaccattaggagtagcacccaccaa
ggcaaagagaagagtggtgcagagagaaaaaagagcagtgggaatagg
agctttgttccttgggttcttgggagcagcaggaagcactatgggcgc
agcgtcaatgacgctgacggtacaggccagacaattattgtctggtat
agtgcagcagcagaacaatttgctgagggctattgaggcgcaacagca
tctgttgcaactcacagtctggggcatcaagcagctccaggcaagaat
cctggctgtggaaagatacctaaaggatcaacagctcctggggatttg
gggttgctctggaaaactcatttgcaccactgctgtgccttggaatgc
tagttggagtaataaatctctggaacagatttggaatcacacgacctg
gatggagtgggacagagaaattaacaattacacaagcttaatacactc
cttaattgaagaatcgcaaaaccagcaagaaaagaatgaacaagaatt
attggaattagataaatgggcaagtttgtggaattggtttaacataac
aaattggctgtggtatataaaattattcataatgatagtaggaggctt
ggtaggtttaagaatagtttttgctgtactttctatagtgaatagagt
taggcagggatattcaccattatcgtttcagacccacctcccaacccc
gaggggacccgacaggcccgaaggaatagaagaagaaggtggagagag
agacagagacagatccattcgattagtgaacggatcggcactgcgtgc
gccaattctgcagacaaatggcagtattcatccacaattttaaaagaa
aaggggggattggggggtacagtgcaggggaaagaatagtagacataa
tagcaacagacatacaaactaaagaattacaaaaacaaattacaaaaa
ttcaaaattttcgggtttattacagggacagcagagatccagtttggt
taattaatggggggacgttaacggggcggaacggtaccgagggcctat
ttcccatgattccttcatatttgcatatacgatacaaggctgttagag
agataattagaattaatttgactgtaaacacaaagatattagtacaaa
atacgtgacgtagaaagtaataatttcttgggtagtttgcagttttaa
aattatgttttaaaatggactatcatatgcttaccgtaacttgaaagt
atttcgatttcttggctttatatatcttgtggaaaggacgaaacaccg
actgggatgtaagccatagcgttttagagctagaaatagcaagttaaa
ataaggctagtccgttatcaacttgaaaaagtggcaccgagtcggtgc
ttttttgaattcgctagctaggtcttgaaaggagtgggaattggctcc
ggtgcccgtcagtgggcagagcgcacatcgcccacagtccccgagaag
ttggggggaggggtcggcaattgatccggtgcctagagaaggtggcgc
ggggtaaactgggaaagtgatgtcgtgtactggctccgcctttttccc
gaggggggggagaaccgtatataagtgcagtagtcgccgtgaacgttc
tttttcgcaacgggtttgccgccagaacacaggaccggtgccaccatg
gactataaggaccacgacggagactacaaggatcatgatattgattac
aaagacgatgacgataagatggccccaaagaagaagcggaaggtcggt
atccacggagtcccagcagccgacaagaagtacagcatcggcctggcc
atcggcaccaactctgtgggctgggccgtgatcaccgacgagtacaag
gtgcccagcaagaaattcaaggtgctgggcaacaccgaccggcacagc
atcaagaagaacctgatcggagccctgctgttcgacagcggcgaaaca
gccgaggccacccggctgaagagaaccgccagaagaagatacaccaga
cggaagaaccggatctgctatctgcaagagatcttcagcaacgagatg
gccaaggtggacgacagcttcttccacagactggaagagtccttcctg
gtggaagaggataagaagcacgagcggcaccccatcttcggcaacatc
gtggacgaggtggcctaccacgagaagtaccccaccatctaccacctg
agaaagaaactggtggacagcaccgacaaggccgacctgcggctgatc
tatctggccctggcccacatgatcaagttccggggccacttcctgatc
gagggcgacctgaaccccgacaacagcgacgtggacaagctgttcatc
cagctggtgcagacctacaaccagctgttcgaggaaaaccccatcaac
gccagcggcgtggacgccaaggccatcctgtctgccagactgagcaag
agcagacggctggaaaatctgatcgcccagctgcccggcgagaagaag
aatggcctgttcggcaacctgattgccctgagcctgggcctgaccccc
aacttcaagagcaacttcgacctggccgaggatgccaaactgcagctg
agcaaggacacctacgacgacgacctggacaacctgctggcccagatc
ggcgaccagtacgccgacctgtttctggccgccaagaacctgtccgac
gccatcctgctgagcgacatcctgagagtgaacaccgagatcaccaag
gcccccctgagcgcctctatgatcaagagatacgacgagcaccaccag
gacctgaccctgctgaaagctctcgtgcggcagcagctgcctgagaag
tacaaagagattttcttcgaccagagcaagaacggctacgccggctac
attgacggcggagccagccaggaagagttctacaagttcatcaagccc
atcctggaaaagatggacggcaccgaggaactgctcgtgaagctgaac
agagaggacctgctgcggaagcagcggaccttcgacaacggcagcatc
ccccaccagatccacctgggagagctgcacgccattctgcggcggcag
gaagatttttacccattcctgaaggacaaccgggaaaagatcgagaag
atcctgaccttccgcatcccctactacgtgggccctctggccagggga
aacagcagattcgcctggatgaccagaaagagcgaggaaaccatcacc
ccctggaacttcgaggaagtggtggacaagggcgcttccgcccagagc
ttcatcgagcggatgaccaacttcgataagaacctgcccaacgagaag
gtgctgcccaagcacagcctgctgtacgagtacttcaccgtgtataac
gagctgaccaaagtgaaatacgtgaccgagggaatgagaaagcccgcc
ttcctgagcggcgagcagaaaaaggccatcgtggacctgctgttcaag
accaaccggaaagtgaccgtgaagcagctgaaagaggactacttcaag
aaaatcgagtgcttcgactccgtggaaatctccggcgtggaagatcgg
ttcaacgcctccctgggcacataccacgatctgctgaaaattatcaag
gacaaggacttcctggacaatgaggaaaacgaggacattctggaagat
atcgtgctgaccctgacactgtttgaggacagagagatgatcgaggaa
cggctgaaaacctatgcccacctgttcgacgacaaagtgatgaagcag
ctgaagcggcggagatacaccggctggggcaggctgagccggaagctg
atcaacggcatccgggacaagcagtccggcaagacaatcctggatttc
ctgaagtccgacggcttcgccaacagaaacttcatgcagctgatccac
gacgacagcctgacctttaaagaggacatccagaaagcccaggtgtcc
ggccagggcgatagcctgcacgagcacattgccaatctggccggcagc
cccgccattaagaagggcatcctgcagacagtgaaggtggtggacgag
ctcgtgaaagtgatgggccggcacaagcccgagaacatcgtgatcgaa
atggccagagagaaccagaccacccagaagggacagaagaacagccgc
gagagaatgaagcggatcgaagagggcatcaaagagctgggcagccag
atcctgaaagaacaccccgtggaaaacacccagctgcagaacgagaag
ctgtacctgtactacctgcagaatggggggatatgtacgtggaccagg
aactggacatcaaccggctgtccgactacgatgtggacgctatcgtgc
ctcagagctttctgaaggacgactccatcgacaacaaggtgctgacca
gaagcgacaagaaccggggcaagagcgacaacgtgccctccgaagagg
tcgtgaagaagatgaagaactactggcggcagctgctgaacgccaagc
tgattacccagagaaagttcgacaatctgaccaaggccgagagaggcg
gcctgagcgaactggataaggccggcttcatcaagagacagctggtgg
aaacccggcagatcacaaagcacgtggcacagatcctggactcccgga
tgaacactaagtacgacgagaatgacaagctgatccgggaagtgaaag
tgatcaccctgaagtccaagctggtgtccgatttccggaaggatttcc
agttttacaaagtgcgcgagatcaacaactaccaccacgcccacgacg
cctacctgaacgccgtcgtgggaaccgccctgatcaaaaagtacccta
agctggaaagcgagttcgtgtacggcgactacaaggtgtacgacgtgc
ggaagatgatcgccaagagcgagcaggaaatcggcaaggctaccgcca
agtacttcttctacagcaacatcatgaactttttcaagaccgagatta
ccctggccaacggcgagatccggaagcggcctctgatcgagacaaacg
gcgaaaccggggagatcgtgtgggataagggccgggattttgccaccg
tgcggaaagtgctgagcatgccccaagtgaatatcgtgaaaaagaccg
aggtgcagacaggcggcttcagcaaagagtctatcctgcccaagagga
acagcgataagctgatcgccagaaagaaggactgggaccctaagaagt
acggcggcttcgacagccccaccgtggcctattctgtgctggtggtgg
ccaaagtggaaaagggcaagtccaagaaactgaagagtgtgaaagagc
tgctggggatcaccatcatggaaagaagcagcttcgagaagaatccca
tcgactttctggaagccaagggctacaaagaagtgaaaaaggacctga
tcatcaagctgcctaagtactccctgttcgagctggaaaacggccgga
agagaatgctggcctctgccggcgaactgcagaagggaaacgaactgg
ccctgccctccaaatatgtgaacttcctgtacctggccagccactatg
agaagctgaagggctcccccgaggataatgagcagaaacagctgtttg
tggaacagcacaagcactacctggacgagatcatcgagcagatcagcg
agttctccaagagagtgatcctggccgacgctaatctggacaaagtgc
tgtccgcctacaacaagcaccgggataagcccatcagagagcaggccg
agaatatcatccacctgtttaccctgaccaatctgggagcccctgccg
ccttcaagtactttgacaccaccatcgaccggaagaggtacaccagca
ccaaagaggtgctggacgccaccctgatccaccagagcatcaccggcc
tgtacgagacacggatcgacctgtctcagctgggaggcgacaaaaggc
cggcggccacgaaaaaggccggacaggccaaaaagaaaaagctcgagg
gcggaggcgggagcggatccccctcccggctccagatgttcttcgcta
ataaccacgaccaggaatttgaccctccaaaggtttacccacctgtcc
cagctgagaagaggaagcccatccgggtgctgtctctctttgatggaa
tcgctacagggctcctggtgctgaaggacttgggcattcaggtggacc
gctacattgcctcggaggtgtgtgaggactccatcacggtgggcatgg
tgcggcaccaggggaagatcatgtacgtcggggacgtccgcagcgtca
cacagaagcatatccaggagtggggcccattcgatctggtgattgggg
gcagtccctgcaatgacctctccatcgtcaaccctgctcgcaagggcc
tctacgagggcactggccggctcttctttgagttctaccgcctcctgc
atgatgcgcggcccaaggagggagatgatcgccccttcttctggctct
ttgcgaatgtggtggccatgggcgttagtgacaagagggacatctcgc
gatttctcgagtccaaccctgtgatgattgatgccaaagaagtgtcag
ctgcacacagggcccgctacttctggggtaaccttcccggtatgaaca
ggccgttggcatccactgtgaatgataagctggagctgcaggagtgtc
tggagcatggcaggatagccaagttcagcaaagtgaggaccattacta
cgaggtcaaactccataaagcagggcaaagaccagcattttcctgtgt
tcatgaatgagaaagaggacatcttatggtgcactgaaatggaaaggg
tatttggtttcccagtccactatactgacgtgtccaacatgagccgct
tggcgaggcagagactgctgggccggtcatggagcgtgccagtcatcc
gccacctcttcgctccgctgaaggagtattttgcgtgtgtgtccggcc
ggcccggatccggcgcaacaaacttctctctgctgaaacaagccggag
atgtcgaagagaatcctggaccgaccgagtacaagcccacggtgcgcc
tcgccacccgcgacgacgtccccagggccgtacgcaccctcgccgccg
cgttcgccgactaccccgccacgcgccacaccgtcgatccggaccgcc
acatcgagcgggtcaccgagctgcaagaactcttcctcacgcgcgtcg
ggctcgacatcggcaaggtgtgggtcgcggacgacggcgccgcggtgg
cggtctggaccacgccggagagcgtcgaagcgggggcggtgttcgccg
agatcggcccgcgcatggccgagttgagcggttcccggctggccgcgc
agcaacagatggaaggcctcctggcgccgcaccggcccaaggagcccg
cgtggttcctggccaccgtcggagtctcgcccgaccaccagggcaagg
gtctgggcagcgccgtcgtgctccccggagtggaggcggccgagcgcg
ccggggtgcccgccttcctggagacctccgcgccccgcaacctcccct
tctacgagcggctcggcttcaccgtcaccgccgacgtcgaggtgcccg
aaggaccgcgcacctggtgcatgacccgcaagcccggtgcctgaacgc
gttaagtcgacaatcaacctctggattacaaaatttgtgaaagattga
ctggtattcttaactatgttgctccttttacgctatgtggatacgctg
ctttaatgcctttgtatcatgctattgcttcccgtatggctttcattt
tctcctccttgtataaatcctggttgctgtctctttatgaggagttgt
ggcccgttgtcaggcaacgtggcgtggtgtgcactgtgtttgctgacg
caacccccactggttggggcattgccaccacctgtcagctcctttccg
ggactttcgctttccccctccctattgccacggcggaactcatcgccg
cctgccttgcccgctgctggacaggggctcggctgttgggcactgaca
attccgtggtgttgtcggggaaatcatcgtcctttccttggctgctcg
cctgtgttgccacctggattctgcgcgggacgtccttctgctacgtcc
cttcggccctcaatccagcggaccttccttcccgcggcctgctgccgg
ctctgcggcctcttccgcgtcttcgccttcgccctcagacgagtcgga
tctccctttgggccgcctccccgcgtcgactttaagaccaatgactta
caaggcagctgtagatcttagccactttttaaaagaaaaggggggact
ggaagggctaattcactcccaacgaagacaagatctgctttttgcttg
tactgggtctctctggttagaccagatctgagcctgggagctctctgg
ctaactagggaacccactgcttaagcctcaataaagcttgccttgagt
gcttcaagtagtgtgtgcccgtctgttgtgtgactctggtaactagag
atccctcagacccttttagtcagtgtggaaaatctctagcagggcccg
tttaaacccgctgatcagcctcgactgtgccttctagttgccagccat
ctgttgtttgcccctcccccgtgccttccttgaccctggaaggtgcca
ctcccactgtcctttcctaataaaatgaggaaattgcatcgcattgtc
tgagtaggtgtcattctattctggggggtggggggggcaggacagcaa
gggggaggattgggaagacaatagcaggcatgctggggatgcggtggg
ctctatggcttctgaggcggaaagaaccagctggggctctagggggta
tccccacgcgccctgtagcggcgcattaagcgcggcgggtgtggtggt
tacgcgcagcgtgaccgctacacttgccagcgccctagcgcccgctcc
tttcgctttcttcccttcctttctcgccacgttcgccggctttccccg
tcaagctctaaatcgggggctccctttagggttccgatttagtgcttt
acggcacctcgaccccaaaaaacttgattagggtgatggttcacgtag
tgggccatcgccctgatagacggtttttcgccctttgacgttggagtc
cacgttctttaatagtggactcttgttccaaactggaacaacactcaa
ccctatctcggtctattcttttgatttataagggattttgccgatttc
ggcctattggttaaaaaatgagctgatttaacaaaaatttaacgcgaa
ttaattctgtggaatgtgtgtcagttagggtgtggaaagtccccaggc
tccccagcaggcagaagtatgcaaagcatgcatctcaattagtcagca
accaggtgtggaaagtccccaggctccccagcaggcagaagtatgcaa
agcatgcatctcaattagtcagcaaccatagtcccgcccctaactccg
cccatcccgcccctaactccgcccagttccgcccattctccgccccat
ggctgactaattttttttatttatgcagaggccgaggccgcctctgcc
tctgagctattccagaagtagtgaggaggcttttttggaggcctaggc
ttttgcaaaaagctcccgggagcttgtatatccattttcggatctgat
cagcacgtgttgacaattaatcatcggcatagtatatcggcatagtat
aatacgacaaggtgaggaactaaaccatggccaagttgaccagtgccg
ttccggtgctcaccgcgcgcgacgtcgccggagcggtcgagttctgga
ccgaccggctcgggttctcccgggacttcgtggaggacgacttcgccg
gtgtggtccgggacgacgtgaccctgttcatcagcgcggtccaggacc
aggtggtgccggacaacaccctggcctgggtgtgggtgcgcggcctgg
acgagctgtacgccgagtggtcggaggtcgtgtccacgaacttccggg
acgcctccgggccggccatgaccgagatcggcgagcagccgtgggggg
ggagttcgccctgcgcgacccggccggcaactgcgtgcacttcgtggc
cgaggagcaggactgacacgtgctacgagatttcgattccaccgccgc
cttctatgaaaggttgggcttcggaatcgttttccgggacgccggctg
gatgatcctccagcgcggggatctcatgctggagttcttcgcccaccc
caacttgtttattgcagcttataatggttacaaataaagcaatagcat
cacaaatttcacaaataaagcatttttttcactgcattctagttgtgg
tttgtccaaactcatcaatgtatcttatcatgtctgtataccgtcgac
ctctagctagagcttggcgtaatcatggtcatagctgtttcctgtgtg
aaattgttatccgctcacaattccacacaacatacgagccggaagcat
aaagtgtaaagcctggggtgcctaatgagtgagctaactcacattaat
tgcgttgcgctcactgcccgctttccagtcgggaaacctgtcgtgcca
gctgcattaatgaatcggccaacgcgcggggagaggcggtttgcgtat
tgggcgctcttccgcttcctcgctcactgactcgctgcgctcggtcgt
tcggctgcggcgagcggtatcagctcactcaaaggcggtaatacggtt
atccacagaatcaggggataacgcaggaaagaacatgtgagcaaaagg
ccagcaaaaggccaggaaccgtaaaaaggccgcgttgctggcgttttt
ccataggctccgcccccctgacgagcatcacaaaaatcgacgctcaag
tcagaggtggcgaaacccgacaggactataaagataccaggcgtttcc
ccctggaagctccctcgtgcgctctcctgttccgaccctgccgcttac
cggatacctgtccgcctttctcccttcgggaagcgtggcgctttctca
tagctcacgctgtaggtatctcagttcggtgtaggtcgttcgctccaa
gctgggctgtgtgcacgaaccccccgttcagcccgaccgctgcgcctt
atccggtaactatcgtcttgagtccaacccggtaagacacgacttatc
gccactggcagcagccactggtaacaggattagcagagcgaggtatgt
aggcggtgctacagagttcttgaagtggtggcctaactacggctacac
tagaagaacagtatttggtatctgcgctctgctgaagccagttacctt
cggaaaaagagttggtagctcttgatccggcaaacaaaccaccgctgg
tagcggtggtttttttgtttgcaagcagcagattacgcgcagaaaaaa
aggatctcaagaagatcctttgatcttttctacggggtctgacgctca
gtggaacgaaaactcacgttaagggattttggtcatgagattatcaaa
aaggatcttcacctagatccttttaaattaaaaatgaagttttaaatc
aatctaaagtatatatgagtaaacttggtctgacagttaccaatgctt
aatcagtgaggcacctatctcagcgatctgtctatttcgttcatccat
agttgcctgactccccgtcgtgtagataactacgatacgggagggctt
accatctggccccagtgctgcaatgataccgcgagacccacgctcacc
ggctccagatttatcagcaataaaccagccagccggaagggccgagcg
cagaagtggtcctgcaactttatccgcctccatccagtctattaattg
ttgccgggaagctagagtaagtagttcgccagttaatagtttgcgcaa
cgttgttgccattgctacaggcatcgtggtgtcacgctcgtcgtttgg
tatggcttcattcagctccggttcccaacgatcaaggcgagttacatg
atcccccatgttgtgcaaaaaagcggttagctccttcggtcctccgat
cgttgtcagaagtaagttggccgcagtgttatcactcatggttatggc
agcactgcataattctcttactgtcatgccatccgtaagatgcttttc
tgtgactggtgagtactcaaccaagtcattctgagaatagtgtatgcg
gcgaccgagttgctcttgcccggcgtcaatacgggataataccgcgcc
acatagcagaactttaaaagtgctcatcattggaaaacgttcttcggg
gcgaaaactctcaaggatcttaccgctgttgagatccagttcgatgta
acccactcgtgcacccaactgatcttcagcatcttttactttcaccag
cgtttctgggtgagcaaaaacaggaaggcaaaatgccgcaaaaaaggg
aataagggcgacacggaaatgttgaatactcatactcttcctttttca
atattattgaagcatttatcagggttattgtctcatgagcggatacat
atttgaatgtatttagaaaaataaacaaataggggttccgcgcacatt
tccccgaaaagtgccacctgac.

In an aspect, a disclosed pBK1033 plasmid can comprise the sequence set forth below or a sequence having 70%, 75%, 80%, 85%, 90%, 95%, or more identity to the sequence set forth

(SEQ ID NO: 36)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggggagtatttacggtaaactgcccacttggcagtacatcaagt
gtatcatatgccaagtacgccccctattgacgtcaatgacggtaaatg
gcccgcctggcattatgcccagtacatgaccttatgggactttcctac
ttggcagtacatctacgtattagtcatcgctattaccatggtgatgcg
gttttggcagtacatcaatgggcgtggatagcggtttgactcacgggg
atttccaagtctccaccccattgacgtcaatgggagtttgttttggca
ccaaaatcaacgggactttccaaaatgtcgtaacaactccgccccatt
gacgcaaatgggcggtaggcgtgtacggtgggaggtctatataagcag
cgcgttttgcctgtactgggtctctctggttagaccagatctgagcct
gggagctctctggctaactagggaacccactgcttaagcctcaataaa
gcttgccttgagtgcttcaagtagtgtgtgcccgtctgttgtgtgact
ctggtaactagagatccctcagacccttttagtcagtgtggaaaatct
ctagcagtggcgcccgaacagggacttgaaagcgaaagggaaaccaga
ggagctctctcgacgcaggactcggcttgctgaagcgcgcacggcaag
aggcgaggggcggcgactggtgagtacgccaaaaattttgactagcgg
aggctagaaggagagagatgggtgcgagagcgtcagtattaagcgggg
gagaattagatcgcgatgggaaaaaattcggttaaggccagggggaaa
gaaaaaatataaattaaaacatatagtatgggcaagcagggagctaga
acgattcgcagttaatcctggcctgttagaaacatcagaaggctgtag
acaaatactgggacagctacaaccatcccttcagacaggatcagaaga
acttagatcattatataatacagtagcaaccctctattgtgtgcatca
aaggatagagataaaagacaccaaggaagctttagacaagatagagga
agagcaaaacaaaagtaagaccaccgcacagcaagcggccgctgatct
tcagacctggaggaggagatatgagggacaattggagaagtgaattat
ataaatataaagtagtaaaaattgaaccattaggagtagcacccacca
aggcaaagagaagagtggtgcagagagaaaaaagagcagtgggaatag
gagctttgttccttgggttcttgggagcagcaggaagcactatgggcg
cagcgtcaatgacgctgacggtacaggccagacaattattgtctggta
tagtgcagcagcagaacaatttgctgagggctattgaggcgcaacagc
atctgttgcaactcacagtctggggcatcaagcagctccaggcaagaa
tcctggctgtggaaagatacctaaaggatcaacagctcctggggattt
ggggttgctctggaaaactcatttgcaccactgctgtgccttggaatg
ctagttggagtaataaatctctggaacagatttggaatcacacgacct
ggatggagtgggacagagaaattaacaattacacaagcttaatacact
ccttaattgaagaatcgcaaaaccagcaagaaaagaatgaacaagaat
tattggaattagataaatgggcaagtttgtggaattggtttaacataa
caaattggctgtggtatataaaattattcataatgatagtaggaggct
tggtaggtttaagaatagtttttgctgtactttctatagtgaatagag
ttaggcagggatattcaccattatcgtttcagacccacctcccaaccc
cgaggggacccgacaggcccgaaggaatagaagaagaaggtggagaga
gagacagagacagatccattcgattagtgaacggatcggcactgcgtg
cgccaattctgcagacaaatggcagtattcatccacaattttaaaaga
aaaggggggattggggggtacagtgcaggggaaagaatagtagacata
atagcaacagacatacaaactaaagaattacaaaaacaaattacaaaa
attcaaaattttcgggtttattacagggacagcagagatccagtttgg
ttaattaatggggggacgttaacggggcggaacggtaccgagggccta
tttcccatgattccttcatatttgcatatacgatacaaggctgttaga
gagataattagaattaatttgactgtaaacacaaagatattagtacaa
aatacgtgacgtagaaagtaataatttcttgggtagtttgcagtttta
aaattatgttttaaaatggactatcatatgcttaccgtaacttgaaag
tatttcgatttcttggctttatatatcttgtggaaaggacgaaacacc
ggttggagcttagaatgtgaagttttagagctagaaatagcaagttaa
aataaggctagtccgttatcaacttgaaaaagtggcaccgagtcggtg
cttttttgaattcgctagctaggtcttgaaaggagtgggaattggctc
cggtgcccgtcagtgggcagagcgcacatcgcccacagtccccgagaa
gttggggggaggggtcggcaattgatccggtgcctagagaaggtggcg
cggggtaaactgggaaagtgatgtcgtgtactggctccgcctttttcc
cgaggggggggagaaccgtatataagtgcagtagtcgccgtgaacgtt
ctttttcgcaacgggtttgccgccagaacacaggaccggtgccaccat
ggactataaggaccacgacggagactacaaggatcatgatattgatta
caaagacgatgacgataagatggccccaaagaagaagcggaaggtcgg
tatccacggagtcccagcagccgacaagaagtacagcatcggcctggc
catcggcaccaactctgtgggctgggccgtgatcaccgacgagtacaa
ggtgcccagcaagaaattcaaggtgctgggcaacaccgaccggcacag
catcaagaagaacctgatcggagccctgctgttcgacagcggcgaaac
agccgaggccacccggctgaagagaaccgccagaagaagatacaccag
acggaagaaccggatctgctatctgcaagagatcttcagcaacgagat
ggccaaggtggacgacagcttcttccacagactggaagagtccttcct
ggtggaagaggataagaagcacgagcggcaccccatcttcggcaacat
cgtggacgaggtggcctaccacgagaagtaccccaccatctaccacct
gagaaagaaactggtggacagcaccgacaaggccgacctgcggctgat
ctatctggccctggcccacatgatcaagttccggggccacttcctgat
cgagggcgacctgaaccccgacaacagcgacgtggacaagctgttcat
ccagctggtgcagacctacaaccagctgttcgaggaaaaccccatcaa
cgccagcggcgtggacgccaaggccatcctgtctgccagactgagcaa
gagcagacggctggaaaatctgatcgcccagctgcccggcgagaagaa
gaatggcctgttcggcaacctgattgccctgagcctgggcctgacccc
caacttcaagagcaacttcgacctggccgaggatgccaaactgcagct
gagcaaggacacctacgacgacgacctggacaacctgctggcccagat
cggcgaccagtacgccgacctgtttctggccgccaagaacctgtccga
cgccatcctgctgagcgacatcctgagagtgaacaccgagatcaccaa
ggcccccctgagcgcctctatgatcaagagatacgacgagcaccacca
ggacctgaccctgctgaaagctctcgtgcggcagcagctgcctgagaa
gtacaaagagattttcttcgaccagagcaagaacggctacgccggcta
cattgacggcggagccagccaggaagagttctacaagttcatcaagcc
catcctggaaaagatggacggcaccgaggaactgctcgtgaagctgaa
cagagaggacctgctgcggaagcagcggaccttcgacaacggcagcat
cccccaccagatccacctgggagagctgcacgccattctgcggcggca
ggaagatttttacccattcctgaaggacaaccgggaaaagatcgagaa
gatcctgaccttccgcatcccctactacgtgggccctctggccagggg
aaacagcagattcgcctggatgaccagaaagagcgaggaaaccatcac
cccctggaacttcgaggaagtggtggacaagggcgcttccgcccagag
cttcatcgagcggatgaccaacttcgataagaacctgcccaacgagaa
ggtgctgcccaagcacagcctgctgtacgagtacttcaccgtgtataa
cgagctgaccaaagtgaaatacgtgaccgagggaatgagaaagcccgc
cttcctgagcggcgagcagaaaaaggccatcgtggacctgctgttcaa
gaccaaccggaaagtgaccgtgaagcagctgaaagaggactacttcaa
gaaaatcgagtgcttcgactccgtggaaatctccggcgtggaagatcg
gttcaacgcctccctgggcacataccacgatctgctgaaaattatcaa
ggacaaggacttcctggacaatgaggaaaacgaggacattctggaaga
tatcgtgctgaccctgacactgtttgaggacagagagatgatcgagga
acggctgaaaacctatgcccacctgttcgacgacaaagtgatgaagca
gctgaagcggcggagatacaccggctggggcaggctgagccggaagct
gatcaacggcatccgggacaagcagtccggcaagacaatcctggattt
cctgaagtccgacggcttcgccaacagaaacttcatgcagctgatcca
cgacgacagcctgacctttaaagaggacatccagaaagcccaggtgtc
cggccagggcgatagcctgcacgagcacattgccaatctggccggcag
ccccgccattaagaagggcatcctgcagacagtgaaggtggtggacga
gctcgtgaaagtgatgggccggcacaagcccgagaacatcgtgatcga
aatggccagagagaaccagaccacccagaagggacagaagaacagccg
cgagagaatgaagcggatcgaagagggcatcaaagagctgggcagcca
gatcctgaaagaacaccccgtggaaaacacccagctgcagaacgagaa
gctgtacctgtactacctgcagaatggggggatatgtacgtggaccag
gaactggacatcaaccggctgtccgactacgatgtggacgctatcgtg
cctcagagctttctgaaggacgactccatcgacaacaaggtgctgacc
agaagcgacaagaaccggggcaagagcgacaacgtgccctccgaagag
gtcgtgaagaagatgaagaactactggcggcagctgctgaacgccaag
ctgattacccagagaaagttcgacaatctgaccaaggccgagagaggc
ggcctgagcgaactggataaggccggcttcatcaagagacagctggtg
gaaacccggcagatcacaaagcacgtggcacagatcctggactcccgg
atgaacactaagtacgacgagaatgacaagctgatccgggaagtgaaa
gtgatcaccctgaagtccaagctggtgtccgatttccggaaggatttc
cagttttacaaagtgcgcgagatcaacaactaccaccacgcccacgac
gcctacctgaacgccgtcgtgggaaccgccctgatcaaaaagtaccct
aagctggaaagcgagttcgtgtacggcgactacaaggtgtacgacgtg
cggaagatgatcgccaagagcgagcaggaaatcggcaaggctaccgcc
aagtacttcttctacagcaacatcatgaactttttcaagaccgagatt
accctggccaacggcgagatccggaagcggcctctgatcgagacaaac
ggcgaaaccggggagatcgtgtgggataagggccgggattttgccacc
gtgcggaaagtgctgagcatgccccaagtgaatatcgtgaaaaagacc
gaggtgcagacaggcggcttcagcaaagagtctatcctgcccaagagg
aacagcgataagctgatcgccagaaagaaggactgggaccctaagaag
tacggcggcttcgacagccccaccgtggcctattctgtgctggtggtg
gccaaagtggaaaagggcaagtccaagaaactgaagagtgtgaaagag
ctgctggggatcaccatcatggaaagaagcagcttcgagaagaatccc
atcgactttctggaagccaagggctacaaagaagtgaaaaaggacctg
atcatcaagctgcctaagtactccctgttcgagctggaaaacggccgg
aagagaatgctggcctctgccggcgaactgcagaagggaaacgaactg
gccctgccctccaaatatgtgaacttcctgtacctggccagccactat
gagaagctgaagggctcccccgaggataatgagcagaaacagctgttt
gtggaacagcacaagcactacctggacgagatcatcgagcagatcagc
gagttctccaagagagtgatcctggccgacgctaatctggacaaagtg
ctgtccgcctacaacaagcaccgggataagcccatcagagagcaggcc
gagaatatcatccacctgtttaccctgaccaatctgggagcccctgcc
gccttcaagtactttgacaccaccatcgaccggaagaggtacaccagc
accaaagaggtgctggacgccaccctgatccaccagagcatcaccggc
ctgtacgagacacggatcgacctgtctcagctgggaggcgacaaaagg
ccggcggccacgaaaaaggccggacaggccaaaaagaaaaagctcgag
ggcggaggcgggagcggatccccctcccggctccagatgttcttcgct
aataaccacgaccaggaatttgaccctccaaaggtttacccacctgtc
ccagctgagaagaggaagcccatccgggtgctgtctctctttgatgga
atcgctacagggctcctggtgctgaaggacttgggcattcaggtggac
cgctacattgcctcggaggtgtgtgaggactccatcacggtgggcatg
gtgcggcaccaggggaagatcatgtacgtcggggacgtccgcagcgtc
acacagaagcatatccaggagtggggcccattcgatctggtgattggg
ggcagtccctgcaatgacctctccatcgtcaaccctgctcgcaagggc
ctctacgagggcactggccggctcttctttgagttctaccgcctcctg
catgatgcgcggcccaaggagggagatgatcgccccttcttctggctc
tttgcgaatgtggtggccatgggcgttagtgacaagagggacatctcg
cgatttctcgagtccaaccctgtgatgattgatgccaaagaagtgtca
gctgcacacagggcccgctacttctggggtaaccttcccggtatgaac
aggccgttggcatccactgtgaatgataagctggagctgcaggagtgt
ctggagcatggcaggatagccaagttcagcaaagtgaggaccattact
acgaggtcaaactccataaagcagggcaaagaccagcattttcctgtg
ttcatgaatgagaaagaggacatcttatggtgcactgaaatggaaagg
gtatttggtttcccagtccactatactgacgtgtccaacatgagccgc
ttggcgaggcagagactgctgggccggtcatggagcgtgccagtcatc
cgccacctcttcgctccgctgaaggagtattttgcgtgtgtgtccggc
cggcccggatccggcgcaacaaacttctctctgctgaaacaagccgga
gatgtcgaagagaatcctggaccgaccgagtacaagcccacggtgcgc
ctcgccacccgcgacgacgtccccagggccgtacgcaccctcgccgcc
gcgttcgccgactaccccgccacgcgccacaccgtcgatccggaccgc
cacatcgagcgggtcaccgagctgcaagaactcttcctcacgcgcgtc
gggctcgacatcggcaaggtgtgggtcgcggacgacggcgccgcggtg
gcggtctggaccacgccggagagcgtcgaagcgggggcggtgttcgcc
gagatcggcccgcgcatggccgagttgagcggttcccggctggccgcg
cagcaacagatggaaggcctcctggcgccgcaccggcccaaggagccc
gcgtggttcctggccaccgtcggagtctcgcccgaccaccagggcaag
ggtctgggcagcgccgtcgtgctccccggagtggaggcggccgagcgc
gccggggtgcccgccttcctggagacctccgcgccccgcaacctcccc
ttctacgagcggctcggcttcaccgtcaccgccgacgtcgaggtgccc
gaaggaccgcgcacctggtgcatgacccgcaagcccggtgcctgaacg
cgttaagtcgacaatcaacctctggattacaaaatttgtgaaagattg
actggtattcttaactatgttgctccttttacgctatgtggatacgct
gctttaatgcctttgtatcatgctattgcttcccgtatggctttcatt
ttctcctccttgtataaatcctggttgctgtctctttatgaggagttg
tggcccgttgtcaggcaacgtggcgtggtgtgcactgtgtttgctgac
gcaacccccactggttggggcattgccaccacctgtcagctcctttcc
gggactttcgctttccccctccctattgccacggcggaactcatcgcc
gcctgccttgcccgctgctggacaggggctcggctgttgggcactgac
aattccgtggtgttgtcggggaaatcatcgtcctttccttggctgctc
gcctgtgttgccacctggattctgcgcgggacgtccttctgctacgtc
ccttcggccctcaatccagcggaccttccttcccgcggcctgctgccg
gctctgcggcctcttccgcgtcttcgccttcgccctcagacgagtcgg
atctccctttgggccgcctccccgcgtcgactttaagaccaatgactt
acaaggcagctgtagatcttagccactttttaaaagaaaaggggggac
tggaagggctaattcactcccaacgaagacaagatctgctttttgctt
gtactgggtctctctggttagaccagatctgagcctgggagctctctg
gctaactagggaacccactgcttaagcctcaataaagcttgccttgag
tgcttcaagtagtgtgtgcccgtctgttgtgtgactctggtaactaga
gatccctcagacccttttagtcagtgtggaaaatctctagcagggccc
gtttaaacccgctgatcagcctcgactgtgccttctagttgccagcca
tctgttgtttgcccctcccccgtgccttccttgaccctggaaggtgcc
actcccactgtcctttcctaataaaatgaggaaattgcatcgcattgt
ctgagtaggtgtcattctattctggggggtggggggggcaggacagca
agggggaggattgggaagacaatagcaggcatgctggggatgcggtgg
gctctatggcttctgaggcggaaagaaccagctggggctctagggggt
atccccacgcgccctgtagcggcgcattaagcgcggcgggtgtggtgg
ttacgcgcagcgtgaccgctacacttgccagcgccctagcgcccgctc
ctttcgctttcttcccttcctttctcgccacgttcgccggctttcccc
gtcaagctctaaatcgggggctccctttagggttccgatttagtgctt
tacggcacctcgaccccaaaaaacttgattagggtgatggttcacgta
gtgggccatcgccctgatagacggtttttcgccctttgacgttggagt
ccacgttctttaatagtggactcttgttccaaactggaacaacactca
accctatctcggtctattcttttgatttataagggattttgccgattt
cggcctattggttaaaaaatgagctgatttaacaaaaatttaacgcga
attaattctgtggaatgtgtgtcagttagggtgtggaaagtccccagg
ctccccagcaggcagaagtatgcaaagcatgcatctcaattagtcagc
aaccaggtgtggaaagtccccaggctccccagcaggcagaagtatgca
aagcatgcatctcaattagtcagcaaccatagtcccgcccctaactcc
gcccatcccgcccctaactccgcccagttccgcccattctccgcccca
tggctgactaattttttttatttatgcagaggccgaggccgcctctgc
ctctgagctattccagaagtagtgaggaggcttttttggaggcctagg
cttttgcaaaaagctcccgggagcttgtatatccattttcggatctga
tcagcacgtgttgacaattaatcatcggcatagtatatcggcatagta
taatacgacaaggtgaggaactaaaccatggccaagttgaccagtgcc
gttccggtgctcaccgcgcgcgacgtcgccggagcggtcgagttctgg
accgaccggctcgggttctcccgggacttcgtggaggacgacttcgcc
ggtgtggtccgggacgacgtgaccctgttcatcagcgcggtccaggac
caggtggtgccggacaacaccctggcctgggtgtgggtgcgcggcctg
gacgagctgtacgccgagtggtcggaggtcgtgtccacgaacttccgg
gacgcctccgggccggccatgaccgagatcggcgagcagccgtggggg
gggagttcgccctgcgcgacccggccggcaactgcgtgcacttcgtgg
ccgaggagcaggactgacacgtgctacgagatttcgattccaccgccg
ccttctatgaaaggttgggcttcggaatcgttttccgggacgccggct
ggatgatcctccagcgcggggatctcatgctggagttcttcgcccacc
ccaacttgtttattgcagcttataatggttacaaataaagcaatagca
tcacaaatttcacaaataaagcatttttttcactgcattctagttgtg
gtttgtccaaactcatcaatgtatcttatcatgtctgtataccgtcga
cctctagctagagcttggcgtaatcatggtcatagctgtttcctgtgt
gaaattgttatccgctcacaattccacacaacatacgagccggaagca
taaagtgtaaagcctggggtgcctaatgagtgagctaactcacattaa
ttgcgttgcgctcactgcccgctttccagtcgggaaacctgtcgtgcc
agctgcattaatgaatcggccaacgcgcggggagaggcggtttgcgta
ttgggcgctcttccgcttcctcgctcactgactcgctgcgctcggtcg
ttcggctgcggcgagcggtatcagctcactcaaaggcggtaatacggt
tatccacagaatcaggggataacgcaggaaagaacatgtgagcaaaag
gccagcaaaaggccaggaaccgtaaaaaggccgcgttgctggcgtttt
tccataggctccgcccccctgacgagcatcacaaaaatcgacgctcaa
gtcagaggtggcgaaacccgacaggactataaagataccaggcgtttc
cccctggaagctccctcgtgcgctctcctgttccgaccctgccgctta
ccggatacctgtccgcctttctcccttcgggaagcgtggcgctttctc
atagctcacgctgtaggtatctcagttcggtgtaggtcgttcgctcca
agctgggctgtgtgcacgaaccccccgttcagcccgaccgctgcgcct
tatccggtaactatcgtcttgagtccaacccggtaagacacgacttat
cgccactggcagcagccactggtaacaggattagcagagcgaggtatg
taggcggtgctacagagttcttgaagtggtggcctaactacggctaca
ctagaagaacagtatttggtatctgcgctctgctgaagccagttacct
tcggaaaaagagttggtagctcttgatccggcaaacaaaccaccgctg
gtagcggtggtttttttgtttgcaagcagcagattacgcgcagaaaaa
aaggatctcaagaagatcctttgatcttttctacggggtctgacgctc
agtggaacgaaaactcacgttaagggattttggtcatgagattatcaa
aaaggatcttcacctagatccttttaaattaaaaatgaagttttaaat
caatctaaagtatatatgagtaaacttggtctgacagttaccaatgct
taatcagtgaggcacctatctcagcgatctgtctatttcgttcatcca
tagttgcctgactccccgtcgtgtagataactacgatacgggagggct
taccatctggccccagtgctgcaatgataccgcgagacccacgctcac
cggctccagatttatcagcaataaaccagccagccggaagggccgagc
gcagaagtggtcctgcaactttatccgcctccatccagtctattaatt
gttgccgggaagctagagtaagtagttcgccagttaatagtttgcgca
acgttgttgccattgctacaggcatcgtggtgtcacgctcgtcgtttg
gtatggcttcattcagctccggttcccaacgatcaaggcgagttacat
gatcccccatgttgtgcaaaaaagcggttagctccttcggtcctccga
tcgttgtcagaagtaagttggccgcagtgttatcactcatggttatgg
cagcactgcataattctcttactgtcatgccatccgtaagatgctttt
ctgtgactggtgagtactcaaccaagtcattctgagaatagtgtatgc
ggcgaccgagttgctcttgcccggcgtcaatacgggataataccgcgc
cacatagcagaactttaaaagtgctcatcattggaaaacgttcttcgg
ggcgaaaactctcaaggatcttaccgctgttgagatccagttcgatgt
aacccactcgtgcacccaactgatcttcagcatcttttactttcacca
gcgtttctgggtgagcaaaaacaggaaggcaaaatgccgcaaaaaagg
gaataagggcgacacggaaatgttgaatactcatactcttcctttttc
aatattattgaagcatttatcagggttattgtctcatgagcggataca
tatttgaatgtatttagaaaaataaacaaataggggttccgcgcacat
ttccccgaaaagtgccacctgac.

In an aspect, a disclosed pBK1105 plasmid can comprise the sequence set forth below or a sequence having 70%, 75%, 80%, 85%, 90%, 95%, or more identity to the sequence set forth

(SEQ ID NO: 43)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggtggagtatttacggtaaactgcccacttggcagtacatcaag
tgtatcatatgccaagtacgccccctattgacgtcaatgacggtaaat
ggcccgcctggcattatgcccagtacatgaccttatgggactttccta
cttggcagtacatctacgtattagtcatcgctattaccatggtgatgc
ggttttggcagtacatcaatgggcgtggatagcggtttgactcacggg
gatttccaagtctccaccccattgacgtcaatgggagtttgttttggc
accaaaatcaacgggactttccaaaatgtcgtaacaactccgccccat
tgacgcaaatgggcggtaggcgtgtacggtgggaggtctatataagca
gcgcgttttgcctgtactgggtctctctggttagaccagatctgagcc
tgggagctctctggctaactagggaacccactgcttaagcctcaataa
agcttgccttgagtgcttcaagtagtgtgtgcccgtctgttgtgtgac
tctggtaactagagatccctcagacccttttagtcagtgtggaaaatc
tctagcagtggcgcccgaacagggacttgaaagcgaaagggaaaccag
aggagctctctcgacgcaggactcggcttgctgaagcgcgcacggcaa
gaggcgaggggcggcgactggtgagtacgccaaaaattttgactagcg
gaggctagaaggagagagatgggtgcgagagcgtcagtattaagcggg
ggagaattagatcgcgatgggaaaaaattcggttaaggccagggggaa
agaaaaaatataaattaaaacatatagtatgggcaagcagggagctag
aacgattcgcagttaatcctggcctgttagaaacatcagaaggctgta
gacaaatactgggacagctacaaccatcccttcagacaggatcagaag
aacttagatcattatataatacagtagcaaccctctattgtgtgcatc
aaaggatagagataaaagacaccaaggaagctttagacaagatagagg
aagagcaaaacaaaagtaagaccaccgcacagcaagcggccgctgatc
ttcagacctggaggaggagatatgagggacaattggagaagtgaatta
tataaatataaagtagtaaaaattgaaccattaggagtagcacccacc
aaggcaaagagaagagtggtgcagagagaaaaaagagcagtgggaata
ggagctttgttccttgggttcttgggagcagcaggaagcactatgggc
gcagcgtcaatgacgctgacggtacaggccagacaattattgtctggt
atagtgcagcagcagaacaatttgctgagggctattgaggcgcaacag
catctgttgcaactcacagtctggggcatcaagcagctccaggcaaga
atcctggctgtggaaagatacctaaaggatcaacagctcctggggatt
tggggttgctctggaaaactcatttgcaccactgctgtgccttggaat
gctagttggagtaataaatctctggaacagatttggaatcacacgacc
tggatggagtgggacagagaaattaacaattacacaagcttaatacac
tccttaattgaagaatcgcaaaaccagcaagaaaagaatgaacaagaa
ttattggaattagataaatgggcaagtttgtggaattggtttaacata
acaaattggctgtggtatataaaattattcataatgatagtaggaggc
ttggtaggtttaagaatagtttttgctgtactttctatagtgaataga
gttaggcagggatattcaccattatcgtttcagacccacctcccaacc
ccgaggggacccgacaggcccgaaggaatagaagaagaaggtggagag
agagacagagacagatccattcgattagtgaacggatcggcactgcgt
gcgccaattctgcagacaaatggcagtattcatccacaattttaaaag
aaaaggggggattggggggtacagtgcaggggaaagaatagtagacat
aatagcaacagacatacaaactaaagaattacaaaaacaaattacaaa
aattcaaaattttcgggtttattacagggacagcagagatccagtttg
gttaattaatggggggacgttaacggggcggaacggtaccgagggcct
atttcccatgattccttcatatttgcatatacgatacaaggctgttag
agagataattagaattaatttgactgtaaacacaaagatattagtaca
aaatacgtgacgtagaaagtaataatttcttgggtagtttgcagtttt
aaaattatgttttaaaatggactatcatatgcttaccgtaacttgaaa
gtatttcgatttcttggctttatatatcttgtggaaaggacgaaacac
cggccctatccctgggggaggggttttagagctagaaatagcaagtta
aaataaggctagtccgttatcaacttgaaaaagtggcaccgagtcggt
gcttttttgaattcgctagctaggtcttgaaaggagtgggaattggct
ccggtgcccgtcagtgggcagagcgcacatcgcccacagtccccgaga
agttggggggaggggtcggcaattgatccggtgcctagagaaggtggc
gcggggtaaactgggaaagtgatgtcgtgtactggctccgcctttttc
ccgaggggggggagaaccgtatataagtgcagtagtcgccgtgaacgt
tctttttcgcaacgggtttgccgccagaacacaggaccggtgccacca
tggactataaggaccacgacggagactacaaggatcatgatattgatt
acaaagacgatgacgataagatggccccaaagaagaagcggaaggtcg
gtatccacggagtcccagcagccgacaagaagtacagcatcggcctgg
ccatcggcaccaactctgtgggctgggccgtgatcaccgacgagtaca
aggtgcccagcaagaaattcaaggtgctgggcaacaccgaccggcaca
gcatcaagaagaacctgatcggagccctgctgttcgacagcggcgaaa
cagccgaggccacccggctgaagagaaccgccagaagaagatacacca
gacggaagaaccggatctgctatctgcaagagatcttcagcaacgaga
tggccaaggtggacgacagcttcttccacagactggaagagtccttcc
tggtggaagaggataagaagcacgagcggcaccccatcttcggcaaca
tcgtggacgaggtggcctaccacgagaagtaccccaccatctaccacc
tgagaaagaaactggtggacagcaccgacaaggccgacctgcggctga
tctatctggccctggcccacatgatcaagttccggggccacttcctga
tcgagggcgacctgaaccccgacaacagcgacgtggacaagctgttca
tccagctggtgcagacctacaaccagctgttcgaggaaaaccccatca
acgccagcggcgtggacgccaaggccatcctgtctgccagactgagca
agagcagacggctggaaaatctgatcgcccagctgcccggcgagaaga
agaatggcctgttcggcaacctgattgccctgagcctgggcctgaccc
ccaacttcaagagcaacttcgacctggccgaggatgccaaactgcagc
tgagcaaggacacctacgacgacgacctggacaacctgctggcccaga
tcggcgaccagtacgccgacctgtttctggccgccaagaacctgtccg
acgccatcctgctgagcgacatcctgagagtgaacaccgagatcacca
aggcccccctgagcgcctctatgatcaagagatacgacgagcaccacc
aggacctgaccctgctgaaagctctcgtgcggcagcagctgcctgaga
agtacaaagagattttcttcgaccagagcaagaacggctacgccggct
acattgacggcggagccagccaggaagagttctacaagttcatcaagc
ccatcctggaaaagatggacggcaccgaggaactgctcgtgaagctga
acagagaggacctgctgcggaagcagcggaccttcgacaacggcagca
tcccccaccagatccacctgggagagctgcacgccattctgcggcggc
aggaagatttttacccattcctgaaggacaaccgggaaaagatcgaga
agatcctgaccttccgcatcccctactacgtgggccctctggccaggg
gaaacagcagattcgcctggatgaccagaaagagcgaggaaaccatca
ccccctggaacttcgaggaagtggtggacaagggcgcttccgcccaga
gcttcatcgagcggatgaccaacttcgataagaacctgcccaacgaga
aggtgctgcccaagcacagcctgctgtacgagtacttcaccgtgtata
acgagctgaccaaagtgaaatacgtgaccgagggaatgagaaagcccg
ccttcctgagcggcgagcagaaaaaggccatcgtggacctgctgttca
agaccaaccggaaagtgaccgtgaagcagctgaaagaggactacttca
agaaaatcgagtgcttcgactccgtggaaatctccggcgtggaagatc
ggttcaacgcctccctgggcacataccacgatctgctgaaaattatca
aggacaaggacttcctggacaatgaggaaaacgaggacattctggaag
atatcgtgctgaccctgacactgtttgaggacagagagatgatcgagg
aacggctgaaaacctatgcccacctgttcgacgacaaagtgatgaagc
agctgaagcggcggagatacaccggctggggcaggctgagccggaagc
tgatcaacggcatccgggacaagcagtccggcaagacaatcctggatt
tcctgaagtccgacggcttcgccaacagaaacttcatgcagctgatcc
acgacgacagcctgacctttaaagaggacatccagaaagcccaggtgt
ccggccagggcgatagcctgcacgagcacattgccaatctggccggca
gccccgccattaagaagggcatcctgcagacagtgaaggtggtggacg
agctcgtgaaagtgatgggccggcacaagcccgagaacatcgtgatcg
aaatggccagagagaaccagaccacccagaagggacagaagaacagcc
gcgagagaatgaagcggatcgaagagggcatcaaagagctgggcagcc
agatcctgaaagaacaccccgtggaaaacacccagctgcagaacgaga
agctgtacctgtactacctgcagaatggggggatatgtacgtggacca
ggaactggacatcaaccggctgtccgactacgatgtggacgctatcgt
gcctcagagctttctgaaggacgactccatcgacaacaaggtgctgac
cagaagcgacaagaaccggggcaagagcgacaacgtgccctccgaaga
ggtcgtgaagaagatgaagaactactggcggcagctgctgaacgccaa
gctgattacccagagaaagttcgacaatctgaccaaggccgagagagg
cggcctgagcgaactggataaggccggcttcatcaagagacagctggt
ggaaacccggcagatcacaaagcacgtggcacagatactagattcccg
aatgaatacgaaatacgacgagaacgataagctgattcgggaagtcaa
agtaatcactttaaagtcaaaattggtgtcggacttcagaaaggattt
tcaattctataaagttagggagataaataactaccaccatgcgcacga
cgcttatcttaatgccgtcgtagggaccgcactcattaagaaataccc
gaagctagaaagtgagtttgtgtatggtgattacaaagtttatgacgt
ccgtaagatgatcgcgaaaagcgaacaggagataggcaaggctacagc
caaatacttcttttattctaacattatgaatttctttaagacggaaat
cactctggcaaacggagagatacgcaaacgacctttaattgaaaccaa
tggggagacaggtgaaatcgtatgggataagggccgggacttcgcgac
ggtgagaaaagttttgtccatgccccaagtcaacatagtaaagaaaac
tgaggtgcagaccggagggttttcaaaggaatcgattcttccaaaaag
gaatagtgataagctcatcgctcgtaaaaaggactgggacccgaaaaa
gtacggtggcttcgtgagccctacagttgcctattctgtcctagtagt
ggcaaaagttgagaagggaaaatccaagaaactgaagtcagtcaaaga
attattggggataacgattatggagcgctcgtcttttgaaaagaaccc
catcgacttccttgaggcgaaaggttacaaggaagtaaaaaaggatct
cataattaaactaccaaagtatagtctgtttgagttagaaaatggccg
aaaacggatgttggctagcgccagagagcttcaaaaggggaacgaact
cgcactaccgtctaaatacgtgaatttcctgtatttagcgtcccatta
cgagaagttgaaaggttcacctgaagataacgaacagaagcaactttt
tgttgagcagcacaaacattatctcgacgaaatcatagagcaaatttc
ggaattcagtaagagagtcatcctagctgatgccaatctggacaaagt
attaagcgcatacaacaagcacagggataaacccatacgtgagcaggc
ggaaaatattatccatttgtttactcttaccaacctcggcgctccagc
cgcattcaagtattttgacacaacgatagatcgcaaagagtacagatc
taccaaggaggtgctagacgcgacactgattcaccaatccatcacggg
attatatgaaactcggatagatttgtcacagcttgggggtgacggatc
ccccaagaagaagaggaaagtcctcgagggcggaggcgggagcggatc
cccctcccggctccagatgttcttcgctaataaccacgaccaggaatt
tgaccctccaaaggtttacccacctgtcccagctgagaagaggaagcc
catccgggtgctgtctctctttgatggaatcgctacagggctcctggt
gctgaaggacttgggcattcaggtggaccgctacattgcctcggaggt
gtgtgaggactccatcacggtgggcatggtgcggcaccaggggaagat
catgtacgtcggggacgtccgcagcgtcacacagaagcatatccagga
gtggggcccattcgatctggtgattgggggcagtccctgcaatgacct
ctccatcgtcaaccctgctcgcaagggcctctacgagggcactggccg
gctcttctttgagttctaccgcctcctgcatgatgcgcggcccaagga
gggagatgatcgccccttcttctggctctttgagaatgtggtggccat
gggcgttagtgacaagagggacatctcgcgatttctcgagtccaaccc
tgtgatgattgatgccaaagaagtgtcagctgcacacagggcccgcta
cttctggggtaaccttcccggtatgaacaggccgttggcatccactgt
gaatgataagctggagctgcaggagtgtctggagcatggcaggatagc
caagttcagcaaagtgaggaccattactacgaggtcaaactccataaa
gcagggcaaagaccagcattttcctgtgttcatgaatgagaaagagga
catcttatggtgcactgaaatggaaagggtatttggtttcccagtcca
ctatactgacgtgtccaacatgagccgcttggcgaggcagagactgct
gggccggtcatggagcgtgccagtcatccgccacctcttcgctccgct
gaaggagtattttgcgtgtgtgtccggccggcccggatccggcgcaac
aaacttctctctgctgaaacaagccggagatgtcgaagagaatcctgg
accgaccgagtacaagcccacggtgcgcctcgccacccgcgacgacgt
ccccagggccgtacgcaccctcgccgccgcgttcgccgactaccccgc
cacgcgccacaccgtcgatccggaccgccacatcgagcgggtcaccga
gctgcaagaactcttcctcacgcgcgtcgggctcgacatcggcaaggt
gtgggtcgcggacgacggcgccgcggtggcggtctggaccacgccgga
gagcgtcgaagcgggggcggtgttcgccgagatcggcccgcgcatggc
cgagttgagcggttcccggctggccgcgcagcaacagatggaaggcct
cctggcgccgcaccggcccaaggagcccgcgtggttcctggccaccgt
cggagtctcgcccgaccaccagggcaagggtctgggcagcgccgtcgt
gctccccggagtggaggcggccgagcgcgccggggtgcccgccttcct
ggagacctccgcgccccgcaacctccccttctacgagcggctcggctt
caccgtcaccgccgacgtcgaggtgcccgaaggaccgcgcacctggtg
catgacccgcaagcccggtgcctgaacgcgttaagtcgacaatcaacc
tctggattacaaaatttgtgaaagattgactggtattcttaactatgt
tgctccttttacgctatgtggatacgctgctttaatgcctttgtatca
tgctattgcttcccgtatggctttcattttctcctccttgtataaatc
ctggttgctgtctctttatgaggagttgtggcccgttgtcaggcaacg
tggcgtggtgtgcactgtgtttgctgacgcaacccccactggttgggg
cattgccaccacctgtcagctcctttccgggactttcgctttccccct
ccctattgccacggggaactcatcgccgcctgccttgcccgctgctgg
acaggggctcggctgttgggcactgacaattccgtggtgttgtcgggg
aaatcatcgtcctttccttggctgctcgcctgtgttgccacctggatt
ctgcgcgggacgtccttctgctacgtcccttcggccctcaatccagcg
gaccttccttcccgcggcctgctgccggctctgcggcctcttccgcgt
cttcgccttcgccctcagacgagtcggatctccctttgggccgcctcc
ccgcgtcgactttaagaccaatgacttacaaggcagctgtagatctta
gccactttttaaaagaaaaggggggactggaagggctaattcactccc
aacgaagacaagatctgctttttgcttgtactgggtctctctggttag
accagatctgagcctgggagctctctggctaactagggaacccactgc
ttaagcctcaataaagcttgccttgagtgcttcaagtagtgtgtgccc
gtctgttgtgtgactctggtaactagagatccctcagacccttttagt
cagtgtggaaaatctctagcagggcccgtttaaacccgctgatcagcc
tcgactgtgccttctagttgccagccatctgttgtttgcccctccccc
gtgccttccttgaccctggaaggtgccactcccactgtcctttcctaa
taaaatgaggaaattgcatcgcattgtctgagtaggtgtcattctatt
ctggggggtggggggggcaggacagcaagggggaggattgggaagaca
atagcaggcatgctggggatgcggtgggctctatggcttctgaggcgg
aaagaaccagctggggctctagggggtatccccacgcgccctgtagcg
gcgcattaagcgcggcgggtgtggtggttacgcgcagcgtgaccgcta
cacttgccagcgccctagcgcccgctcctttcgctttcttcccttcct
ttctcgccacgttcgccggctttccccgtcaagctctaaatcgggggc
tccctttagggttccgatttagtgctttacggcacctcgaccccaaaa
aacttgattagggtgatggttcacgtagtgggccatcgccctgataga
cggtttttcgccctttgacgttggagtccacgttctttaatagtggac
tcttgttccaaactggaacaacactcaaccctatctcggtctattctt
ttgatttataagggattttgccgatttcggcctattggttaaaaaatg
agctgatttaacaaaaatttaacgcgaattaattctgtggaatgtgtg
tcagttagggtgtggaaagtccccaggctccccagcaggcagaagtat
gcaaagcatgcatctcaattagtcagcaaccaggtgtggaaagtcccc
aggctccccagcaggcagaagtatgcaaagcatgcatctcaattagtc
agcaaccatagtcccgcccctaactccgcccatcccgcccctaactcc
gcccagttccgcccattctccgccccatggctgactaattttttttat
ttatgcagaggccgaggccgcctctgcctctgagctattccagaagta
gtgaggaggcttttttggaggcctaggcttttgcaaaaagctccctac
cgtcgacctctagctagagcttggcgtaatcatggtcatagctgtttc
ctgtgtgaaattgttatccgctcacaattccacacaacatacgagccg
gaagcataaagtgtaaagcctggggtgcctaatgagtgagctaactca
cattaattgcgttgcgctcactgcccgctttccagtcgggaaacctgt
cgtgccagctgcattaatgaatcggccaacgcgcggggagaggcggtt
tgcgtattgggcgctcttccgcttcctcgctcactgactcgctgcgct
cggtcgttcggctgcggcgagcggtatcagctcactcaaaggcggtaa
tacggttatccacagaatcaggggataacgcaggaaagaacatgtgag
caaaaggccagcaaaaggccaggaaccgtaaaaaggccgcgttgctgg
cgtttttccataggctccgcccccctgacgagcatcacaaaaatcgac
gctcaagtcagaggtggcgaaacccgacaggactataaagataccagg
cgtttccccctggaagctccctcgtgcgctctcctgttccgaccctgc
cgcttaccggatacctgtccgcctttctcccttcgggaagcgtggcgc
tttctcatagctcacgctgtaggtatctcagttcggtgtaggtcgttc
gctccaagctgggctgtgtgcacgaaccccccgttcagcccgaccgct
gcgccttatccggtaactatcgtcttgagtccaacccggtaagacacg
acttatcgccactggcagcagccactggtaacaggattagcagagcga
ggtatgtaggcggtgctacagagttcttgaagtggtggcctaactacg
gctacactagaagaacagtatttggtatctgcgctctgctgaagccag
ttaccttcggaaaaagagttggtagctcttgatccggcaaacaaacca
ccgctggtagcggtggtttttttgtttgcaagcagcagattacgcgca
gaaaaaaaggatctcaagaagatcctttgatcttttctacggggtctg
acgctcagtggaacgaaaactcacgttaagggattttggtcatgagat
tatcaaaaaggatcttcacctagatccttttaaattaaaaatgaagtt
ttaaatcaatctaaagtatatatgagtaaacttggtctgacagttacc
aatgcttaatcagtgaggcacctatctcagcgatctgtctatttcgtt
catccatagttgcctgactccccgtcgtgtagataactacgatacggg
agggcttaccatctggccccagtgctgcaatgataccgcgagacccac
gctcaccggctccagatttatcagcaataaaccagccagccggaaggg
ccgagcgcagaagtggtcctgcaactttatccgcctccatccagtcta
ttaattgttgccgggaagctagagtaagtagttcgccagttaatagtt
tgcgcaacgttgttgccattgctacaggcatcgtggtgtcacgctcgt
cgtttggtatggcttcattcagctccggttcccaacgatcaaggcgag
ttacatgatcccccatgttgtgcaaaaaagcggttagctccttcggtc
ctccgatcgttgtcagaagtaagttggccgcagtgttatcactcatgg
ttatggcagcactgcataattctcttactgtcatgccatccgtaagat
gcttttctgtgactggtgagtactcaaccaagtcattctgagaatagt
gtatgcggcgaccgagttgctcttgcccggcgtcaatacgggataata
ccgcgccacatagcagaactttaaaagtgctcatcattggaaaacgtt
cttcggggcgaaaactctcaaggatcttaccgctgttgagatccagtt
cgatgtaacccactcgtgcacccaactgatcttcagcatcttttactt
tcaccagcgtttctgggtgagcaaaaacaggaaggcaaaatgccgcaa
aaaagggaataagggcgacacggaaatgttgaatactcatactcttcc
tttttcaatattattgaagcatttatcagggttattgtctcatgagcg
gatacatatttgaatgtatttagaaaaataaacaaataggggttccgc
gcacatttccccgaaaagtgccacctgac.

In an aspect, a disclosed pBK1106 plasmid can comprise the sequence set forth below or a sequence having 70%, 75%, 80%, 85%, 90%, 95%, or more identity to the sequence set forth

(SEQ ID NO: 44)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggtggagtatttacggtaaactgcccacttggcagtacatcaag
tgtatcatatgccaagtacgccccctattgacgtcaatgacggtaaat
ggcccgcctggcattatgcccagtacatgaccttatgggactttccta
cttggcagtacatctacgtattagtcatcgctattaccatggtgatgc
ggttttggcagtacatcaatgggcgtggatagcggtttgactcacggg
gatttccaagtctccaccccattgacgtcaatgggagtttgttttggc
accaaaatcaacgggactttccaaaatgtcgtaacaactccgccccat
tgacgcaaatgggggtaggcgtgtacggtgggaggtctatataagcag
cgcgttttgcctgtactgggtctctctggttagaccagatctgagcct
gggagctctctggctaactagggaacccactgcttaagcctcaataaa
gcttgccttgagtgcttcaagtagtgtgtgcccgtctgttgtgtgact
ctggtaactagagatccctcagacccttttagtcagtgtggaaaatct
ctagcagtggcgcccgaacagggacttgaaagcgaaagggaaaccaga
ggagctctctcgacgcaggactcggcttgctgaagcgcgcacggcaag
aggcgaggggcggcgactggtgagtacgccaaaaattttgactagcgg
aggctagaaggagagagatgggtgcgagagcgtcagtattaagcgggg
gagaattagatcgcgatgggaaaaaattcggttaaggccagggggaaa
gaaaaaatataaattaaaacatatagtatgggcaagcagggagctaga
acgattcgcagttaatcctggcctgttagaaacatcagaaggctgtag
acaaatactgggacagctacaaccatcccttcagacaggatcagaaga
acttagatcattatataatacagtagcaaccctctattgtgtgcatca
aaggatagagataaaagacaccaaggaagctttagacaagatagagga
agagcaaaacaaaagtaagaccaccgcacagcaagcggccgctgatct
tcagacctggaggaggagatatgagggacaattggagaagtgaattat
ataaatataaagtagtaaaaattgaaccattaggagtagcacccacca
aggcaaagagaagagtggtgcagagagaaaaaagagcagtgggaatag
gagctttgttccttgggttcttgggagcagcaggaagcactatgggcg
cagcgtcaatgacgctgacggtacaggccagacaattattgtctggta
tagtgcagcagcagaacaatttgctgagggctattgaggcgcaacagc
atctgttgcaactcacagtctggggcatcaagcagctccaggcaagaa
tcctggctgtggaaagatacctaaaggatcaacagctcctggggattt
ggggttgctctggaaaactcatttgcaccactgctgtgccttggaatg
ctagttggagtaataaatctctggaacagatttggaatcacacgacct
ggatggagtgggacagagaaattaacaattacacaagcttaatacact
ccttaattgaagaatcgcaaaaccagcaagaaaagaatgaacaagaat
tattggaattagataaatgggcaagtttgtggaattggtttaacataa
caaattggctgtggtatataaaattattcataatgatagtaggaggct
tggtaggtttaagaatagtttttgctgtactttctatagtgaatagag
ttaggcagggatattcaccattatcgtttcagacccacctcccaaccc
cgaggggacccgacaggcccgaaggaatagaagaagaaggtggagaga
gagacagagacagatccattcgattagtgaacggatcggcactgcgtg
cgccaattctgcagacaaatggcagtattcatccacaattttaaaaga
aaaggggggattggggggtacagtgcaggggaaagaatagtagacata
atagcaacagacatacaaactaaagaattacaaaaacaaattacaaaa
attcaaaattttcgggtttattacagggacagcagagatccagtttgg
ttaattaatggggggacgttaacggggggaacggtaccgagggcctat
ttcccatgattccttcatatttgcatatacgatacaaggctgttagag
agataattagaattaatttgactgtaaacacaaagatattagtacaaa
atacgtgacgtagaaagtaataatttcttgggtagtttgcagttttaa
aattatgttttaaaatggactatcatatgcttaccgtaacttgaaagt
atttcgatttcttggctttatatatcttgtggaaaggacgaaacaccg
tcgggcttggggagaggagggttttagagctagaaatagcaagttaaa
ataaggctagtccgttatcaacttgaaaaagtggcaccgagtcggtgc
ttttttgaattcgctagctaggtcttgaaaggagtgggaattggctcc
ggtgcccgtcagtgggcagagcgcacatcgcccacagtccccgagaag
ttggggggaggggtcggcaattgatccggtgcctagagaaggtggcgc
ggggtaaactgggaaagtgatgtcgtgtactggctccgcctttttccc
gaggggggggagaaccgtatataagtgcagtagtcgccgtgaacgttc
tttttcgcaacgggtttgccgccagaacacaggaccggtgccaccatg
gactataaggaccacgacggagactacaaggatcatgatattgattac
aaagacgatgacgataagatggccccaaagaagaagcggaaggtcggt
atccacggagtcccagcagccgacaagaagtacagcatcggcctggcc
atcggcaccaactctgtgggctgggccgtgatcaccgacgagtacaag
gtgcccagcaagaaattcaaggtgctgggcaacaccgaccggcacagc
atcaagaagaacctgatcggagccctgctgttcgacagcggcgaaaca
gccgaggccacccggctgaagagaaccgccagaagaagatacaccaga
cggaagaaccggatctgctatctgcaagagatcttcagcaacgagatg
gccaaggtggacgacagcttcttccacagactggaagagtccttcctg
gtggaagaggataagaagcacgagcggcaccccatcttcggcaacatc
gtggacgaggtggcctaccacgagaagtaccccaccatctaccacctg
agaaagaaactggtggacagcaccgacaaggccgacctgcggctgatc
tatctggccctggcccacatgatcaagttccggggccacttcctgatc
gagggcgacctgaaccccgacaacagcgacgtggacaagctgttcatc
cagctggtgcagacctacaaccagctgttcgaggaaaaccccatcaac
gccagcggcgtggacgccaaggccatcctgtctgccagactgagcaag
agcagacggctggaaaatctgatcgcccagctgcccggcgagaagaag
aatggcctgttcggcaacctgattgccctgagcctgggcctgaccccc
aacttcaagagcaacttcgacctggccgaggatgccaaactgcagctg
agcaaggacacctacgacgacgacctggacaacctgctggcccagatc
ggcgaccagtacgccgacctgtttctggccgccaagaacctgtccgac
gccatcctgctgagcgacatcctgagagtgaacaccgagatcaccaag
gcccccctgagcgcctctatgatcaagagatacgacgagcaccaccag
gacctgaccctgctgaaagctctcgtgcggcagcagctgcctgagaag
tacaaagagattttcttcgaccagagcaagaacggctacgccggctac
attgacggcggagccagccaggaagagttctacaagttcatcaagccc
atcctggaaaagatggacggcaccgaggaactgctcgtgaagctgaac
agagaggacctgctgcggaagcagcggaccttcgacaacggcagcatc
ccccaccagatccacctgggagagctgcacgccattctgcggcggcag
gaagatttttacccattcctgaaggacaaccgggaaaagatcgagaag
atcctgaccttccgcatcccctactacgtgggccctctggccagggga
aacagcagattcgcctggatgaccagaaagagcgaggaaaccatcacc
ccctggaacttcgaggaagtggtggacaagggcgcttccgcccagagc
ttcatcgagcggatgaccaacttcgataagaacctgcccaacgagaag
gtgctgcccaagcacagcctgctgtacgagtacttcaccgtgtataac
gagctgaccaaagtgaaatacgtgaccgagggaatgagaaagcccgcc
ttcctgagcggcgagcagaaaaaggccatcgtggacctgctgttcaag
accaaccggaaagtgaccgtgaagcagctgaaagaggactacttcaag
aaaatcgagtgcttcgactccgtggaaatctccggcgtggaagatcgg
ttcaacgcctccctgggcacataccacgatctgctgaaaattatcaag
gacaaggacttcctggacaatgaggaaaacgaggacattctggaagat
atcgtgctgaccctgacactgtttgaggacagagagatgatcgaggaa
cggctgaaaacctatgcccacctgttcgacgacaaagtgatgaagcag
ctgaagcggcggagatacaccggctggggcaggctgagccggaagctg
atcaacggcatccgggacaagcagtccggcaagacaatcctggatttc
ctgaagtccgacggcttcgccaacagaaacttcatgcagctgatccac
gacgacagcctgacctttaaagaggacatccagaaagcccaggtgtcc
ggccagggcgatagcctgcacgagcacattgccaatctggccggcagc
cccgccattaagaagggcatcctgcagacagtgaaggtggtggacgag
ctcgtgaaagtgatgggccggcacaagcccgagaacatcgtgatcgaa
atggccagagagaaccagaccacccagaagggacagaagaacagccgc
gagagaatgaagcggatcgaagagggcatcaaagagctgggcagccag
atcctgaaagaacaccccgtggaaaacacccagctgcagaacgagaag
ctgtacctgtactacctgcagaatggggggatatgtacgtggaccagg
aactggacatcaaccggctgtccgactacgatgtggacgctatcgtgc
ctcagagctttctgaaggacgactccatcgacaacaaggtgctgacca
gaagcgacaagaaccggggcaagagcgacaacgtgccctccgaagagg
tcgtgaagaagatgaagaactactggcggcagctgctgaacgccaagc
tgattacccagagaaagttcgacaatctgaccaaggccgagagaggcg
gcctgagcgaactggataaggccggcttcatcaagagacagctggtgg
aaacccggcagatcacaaagcacgtggcacagatactagattcccgaa
tgaatacgaaatacgacgagaacgataagctgattcgggaagtcaaag
taatcactttaaagtcaaaattggtgtcggacttcagaaaggattttc
aattctataaagttagggagataaataactaccaccatgcgcacgacg
cttatcttaatgccgtcgtagggaccgcactcattaagaaatacccga
agctagaaagtgagtttgtgtatggtgattacaaagtttatgacgtcc
gtaagatgatcgcgaaaagcgaacaggagataggcaaggctacagcca
aatacttcttttattctaacattatgaatttctttaagacggaaatca
ctctggcaaacggagagatacgcaaacgacctttaattgaaaccaatg
gggagacaggtgaaatcgtatgggataagggccgggacttcgcgacgg
tgagaaaagttttgtccatgccccaagtcaacatagtaaagaaaactg
aggtgcagaccggagggttttcaaaggaatcgattcttccaaaaagga
atagtgataagctcatcgctcgtaaaaaggactgggacccgaaaaagt
acggtggcttcgtgagccctacagttgcctattctgtcctagtagtgg
caaaagttgagaagggaaaatccaagaaactgaagtcagtcaaagaat
tattggggataacgattatggagcgctcgtcttttgaaaagaacccca
tcgacttccttgaggcgaaaggttacaaggaagtaaaaaaggatctca
taattaaactaccaaagtatagtctgtttgagttagaaaatggccgaa
aacggatgttggctagcgccagagagcttcaaaaggggaacgaactcg
cactaccgtctaaatacgtgaatttcctgtatttagcgtcccattacg
agaagttgaaaggttcacctgaagataacgaacagaagcaactttttg
ttgagcagcacaaacattatctcgacgaaatcatagagcaaatttcgg
aattcagtaagagagtcatcctagctgatgccaatctggacaaagtat
taagcgcatacaacaagcacagggataaacccatacgtgagcaggcgg
aaaatattatccatttgtttactcttaccaacctcggcgctccagccg
cattcaagtattttgacacaacgatagatcgcaaagagtacagatcta
ccaaggaggtgctagacgcgacactgattcaccaatccatcacgggat
tatatgaaactcggatagatttgtcacagcttgggggtgacggatccc
ccaagaagaagaggaaagtcctcgagggcggagggggagcggatcccc
ctcccggctccagatgttcttcgctaataaccacgaccaggaatttga
ccctccaaaggtttacccacctgtcccagctgagaagaggaagcccat
ccgggtgctgtctctctttgatggaatcgctacagggctcctggtgct
gaaggacttgggcattcaggtggaccgctacattgcctcggaggtgtg
tgaggactccatcacggtgggcatggtgcggcaccaggggaagatcat
gtacgtcggggacgtccgcagcgtcacacagaagcatatccaggagtg
gggcccattcgatctggtgattgggggcagtccctgcaatgacctctc
catcgtcaaccctgctcgcaagggcctctacgagggcactggccggct
cttctttgagttctaccgcctcctgcatgatgcgcggcccaaggaggg
agatgatcgccccttcttctggctctttgagaatgtggtggccatggg
cgttagtgacaagagggacatctcgcgatttctcgagtccaaccctgt
gatgattgatgccaaagaagtgtcagctgcacacagggcccgctactt
ctggggtaaccttcccggtatgaacaggccgttggcatccactgtgaa
tgataagctggagctgcaggagtgtctggagcatggcaggatagccaa
gttcagcaaagtgaggaccattactacgaggtcaaactccataaagca
gggcaaagaccagcattttcctgtgttcatgaatgagaaagaggacat
cttatggtgcactgaaatggaaagggtatttggtttcccagtccacta
tactgacgtgtccaacatgagccgcttggcgaggcagagactgctggg
ccggtcatggagcgtgccagtcatccgccacctcttcgctccgctgaa
ggagtattttgcgtgtgtgtccggccggcccggatccggcgcaacaaa
cttctctctgctgaaacaagccggagatgtcgaagagaatcctggacc
gaccgagtacaagcccacggtgcgcctcgccacccgcgacgacgtccc
cagggccgtacgcaccctcgccgccgcgttcgccgactaccccgccac
gcgccacaccgtcgatccggaccgccacatcgagcgggtcaccgagct
gcaagaactcttcctcacgcgcgtcgggctcgacatcggcaaggtgtg
ggtcgcggacgacggcgccgcggtggcggtctggaccacgccggagag
cgtcgaagcgggggcggtgttcgccgagatcggcccgcgcatggccga
gttgagcggttcccggctggccgcgcagcaacagatggaaggcctcct
ggcgccgcaccggcccaaggagcccgcgtggttcctggccaccgtcgg
agtctcgcccgaccaccagggcaagggtctgggcagcgccgtcgtgct
ccccggagtggaggcggccgagcgcgccggggtgcccgccttcctgga
gacctccgcgccccgcaacctccccttctacgagcggctcggcttcac
cgtcaccgccgacgtcgaggtgcccgaaggaccgcgcacctggtgcat
gacccgcaagcccggtgcctgaacgcgttaagtcgacaatcaacctct
ggattacaaaatttgtgaaagattgactggtattcttaactatgttgc
tccttttacgctatgtggatacgctgctttaatgcctttgtatcatgc
tattgcttcccgtatggctttcattttctcctccttgtataaatcctg
gttgctgtctctttatgaggagttgtggcccgttgtcaggcaacgtgg
cgtggtgtgcactgtgtttgctgacgcaacccccactggttggggcat
tgccaccacctgtcagctcctttccgggactttcgctttccccctccc
tattgccacggcggaactcatcgccgcctgccttgcccgctgctggac
aggggctcggctgttgggcactgacaattccgtggtgttgtcggggaa
atcatcgtcctttccttggctgctcgcctgtgttgccacctggattct
gcgcgggacgtccttctgctacgtcccttcggccctcaatccagcgga
ccttccttcccgcggcctgctgccggctctgcggcctcttccgcgtct
tcgccttcgccctcagacgagtcggatctccctttgggccgcctcccc
gcgtcgactttaagaccaatgacttacaaggcagctgtagatcttagc
cactttttaaaagaaaagggggactggaagggctaattcactcccaac
gaagacaagatctgctttttgcttgtactgggtctctctggttagacc
agatctgagcctgggagctctctggctaactagggaacccactgctta
agcctcaataaagcttgccttgagtgcttcaagtagtgtgtgcccgtc
tgttgtgtgactctggtaactagagatccctcagacccttttagtcag
tgtggaaaatctctagcagggcccgtttaaacccgctgatcagcctcg
actgtgccttctagttgccagccatctgttgtttgcccctcccccgtg
ccttccttgaccctggaaggtgccactcccactgtcctttcctaataa
aatgaggaaattgcatcgcattgtctgagtaggtgtcattctattctg
gggggtggggggggcaggacagcaagggggaggattgggaagacaata
gcaggcatgctggggatgcggtgggctctatggcttctgaggcggaaa
gaaccagctggggctctagggggtatccccacgcgccctgtagcggcg
cattaagcgcggcgggtgtggtggttacgcgcagcgtgaccgctacac
ttgccagcgccctagcgcccgctcctttcgctttcttcccttcctttc
tcgccacgttcgccggctttccccgtcaagctctaaatcgggggctcc
ctttagggttccgatttagtgctttacggcacctcgaccccaaaaaac
ttgattagggtgatggttcacgtagtgggccatcgccctgatagacgg
tttttcgccctttgacgttggagtccacgttctttaatagtggactct
tgttccaaactggaacaacactcaaccctatctcggtctattcttttg
atttataagggattttgccgatttcggcctattggttaaaaaatgagc
tgatttaacaaaaatttaacgcgaattaattctgtggaatgtgtgtca
gttagggtgtggaaagtccccaggctccccagcaggcagaagtatgca
aagcatgcatctcaattagtcagcaaccaggtgtggaaagtccccagg
ctccccagcaggcagaagtatgcaaagcatgcatctcaattagtcagc
aaccatagtcccgcccctaactccgcccatcccgcccctaactccgcc
cagttccgcccattctccgccccatggctgactaattttttttattta
tgcagaggccgaggccgcctctgcctctgagctattccagaagtagtg
aggaggcttttttggaggcctaggcttttgcaaaaagctccctaccgt
cgacctctagctagagcttggcgtaatcatggtcatagctgtttcctg
tgtgaaattgttatccgctcacaattccacacaacatacgagccggaa
gcataaagtgtaaagcctggggtgcctaatgagtgagctaactcacat
taattgcgttgcgctcactgcccgctttccagtcgggaaacctgtcgt
gccagctgcattaatgaatcggccaacgcgcggggagaggcggtttgc
gtattgggcgctcttccgcttcctcgctcactgactcgctgcgctcgg
tcgttcggctgcggcgagcggtatcagctcactcaaaggcggtaatac
ggttatccacagaatcaggggataacgcaggaaagaacatgtgagcaa
aaggccagcaaaaggccaggaaccgtaaaaaggccgcgttgctggcgt
ttttccataggctccgcccccctgacgagcatcacaaaaatcgacgct
caagtcagaggtggcgaaacccgacaggactataaagataccaggcgt
ttccccctggaagctccctcgtgcgctctcctgttccgaccctgccgc
ttaccggatacctgtccgcctttctcccttcgggaagcgtggcgcttt
ctcatagctcacgctgtaggtatctcagttcggtgtaggtcgttcgct
ccaagctgggctgtgtgcacgaaccccccgttcagcccgaccgctgcg
ccttatccggtaactatcgtcttgagtccaacccggtaagacacgact
tatcgccactggcagcagccactggtaacaggattagcagagcgaggt
atgtaggcggtgctacagagttcttgaagtggtggcctaactacggct
acactagaagaacagtatttggtatctgcgctctgctgaagccagtta
ccttcggaaaaagagttggtagctcttgatccggcaaacaaaccaccg
ctggtagcggtggtttttttgtttgcaagcagcagattacgcgcagaa
aaaaaggatctcaagaagatcctttgatcttttctacggggtctgacg
ctcagtggaacgaaaactcacgttaagggattttggtcatgagattat
caaaaaggatcttcacctagatccttttaaattaaaaatgaagtttta
aatcaatctaaagtatatatgagtaaacttggtctgacagttaccaat
gcttaatcagtgaggcacctatctcagcgatctgtctatttcgttcat
ccatagttgcctgactccccgtcgtgtagataactacgatacgggagg
gcttaccatctggccccagtgctgcaatgataccgcgagacccacgct
caccggctccagatttatcagcaataaaccagccagccggaagggccg
agcgcagaagtggtcctgcaactttatccgcctccatccagtctatta
attgttgccgggaagctagagtaagtagttcgccagttaatagtttgc
gcaacgttgttgccattgctacaggcatcgtggtgtcacgctcgtcgt
ttggtatggcttcattcagctccggttcccaacgatcaaggcgagtta
catgatcccccatgttgtgcaaaaaagcggttagctccttcggtcctc
cgatcgttgtcagaagtaagttggccgcagtgttatcactcatggtta
tggcagcactgcataattctcttactgtcatgccatccgtaagatgct
tttctgtgactggtgagtactcaaccaagtcattctgagaatagtgta
tgcggcgaccgagttgctcttgcccggcgtcaatacgggataataccg
cgccacatagcagaactttaaaagtgctcatcattggaaaacgttctt
cggggcgaaaactctcaaggatcttaccgctgttgagatccagttcga
tgtaacccactcgtgcacccaactgatcttcagcatcttttactttca
ccagcgtttctgggtgagcaaaaacaggaaggcaaaatgccgcaaaaa
agggaataagggcgacacggaaatgttgaatactcatactcttccttt
ttcaatattattgaagcatttatcagggttattgtctcatgagcggat
acatatttgaatgtatttagaaaaataaacaaataggggttccgcgca
catttccccgaaaagtgccacctgac.

In an aspect, a disclosed pBK1107 plasmid can comprise the sequence set forth below or a sequence having 70%, 75%, 80%, 85%, 90%, 95%, or more identity to the sequence set forth

(SEQ ID NO: 45)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggtggagtatttacggtaaactgcccacttggcagtacatcaag
tgtatcatatgccaagtacgccccctattgacgtcaatgacggtaaat
ggcccgcctggcattatgcccagtacatgaccttatgggactttccta
cttggcagtacatctacgtattagtcatcgctattaccatggtgatgc
ggttttggcagtacatcaatgggcgtggatagcggtttgactcacggg
gatttccaagtctccaccccattgacgtcaatgggagtttgttttggc
accaaaatcaacgggactttccaaaatgtcgtaacaactccgccccat
tgacgcaaatgggggtaggcgtgtacggtgggaggtctatataagcag
cgcgttttgcctgtactgggtctctctggttagaccagatctgagcct
gggagctctctggctaactagggaacccactgcttaagcctcaataaa
gcttgccttgagtgcttcaagtagtgtgtgcccgtctgttgtgtgact
ctggtaactagagatccctcagacccttttagtcagtgtggaaaatct
ctagcagtggcgcccgaacagggacttgaaagcgaaagggaaaccaga
ggagctctctcgacgcaggactcggcttgctgaagcgcgcacggcaag
aggcgaggggcggcgactggtgagtacgccaaaaattttgactagcgg
aggctagaaggagagagatgggtgcgagagcgtcagtattaagcgggg
gagaattagatcgcgatgggaaaaaattcggttaaggccagggggaaa
gaaaaaatataaattaaaacatatagtatgggcaagcagggagctaga
acgattcgcagttaatcctggcctgttagaaacatcagaaggctgtag
acaaatactgggacagctacaaccatcccttcagacaggatcagaaga
acttagatcattatataatacagtagcaaccctctattgtgtgcatca
aaggatagagataaaagacaccaaggaagctttagacaagatagagga
agagcaaaacaaaagtaagaccaccgcacagcaagcggccgctgatct
tcagacctggaggaggagatatgagggacaattggagaagtgaattat
ataaatataaagtagtaaaaattgaaccattaggagtagcacccacca
aggcaaagagaagagtggtgcagagagaaaaaagagcagtgggaatag
gagctttgttccttgggttcttgggagcagcaggaagcactatgggcg
cagcgtcaatgacgctgacggtacaggccagacaattattgtctggta
tagtgcagcagcagaacaatttgctgagggctattgaggcgcaacagc
atctgttgcaactcacagtctggggcatcaagcagctccaggcaagaa
tcctggctgtggaaagatacctaaaggatcaacagctcctggggattt
ggggttgctctggaaaactcatttgcaccactgctgtgccttggaatg
ctagttggagtaataaatctctggaacagatttggaatcacacgacct
ggatggagtgggacagagaaattaacaattacacaagcttaatacact
ccttaattgaagaatcgcaaaaccagcaagaaaagaatgaacaagaat
tattggaattagataaatgggcaagtttgtggaattggtttaacataa
caaattggctgtggtatataaaattattcataatgatagtaggaggct
tggtaggtttaagaatagtttttgctgtactttctatagtgaatagag
ttaggcagggatattcaccattatcgtttcagacccacctcccaaccc
cgaggggacccgacaggcccgaaggaatagaagaagaaggtggagaga
gagacagagacagatccattcgattagtgaacggatcggcactgcgtg
cgccaattctgcagacaaatggcagtattcatccacaattttaaaaga
aaaggggggattggggggtacagtgcaggggaaagaatagtagacata
atagcaacagacatacaaactaaagaattacaaaaacaaattacaaaa
attcaaaattttcgggtttattacagggacagcagagatccagtttgg
ttaattaatggggggacgttaacggggcggaacggtaccgagggccta
tttcccatgattccttcatatttgcatatacgatacaaggctgttaga
gagataattagaattaatttgactgtaaacacaaagatattagtacaa
aatacgtgacgtagaaagtaataatttcttgggtagtttgcagtttta
aaattatgttttaaaatggactatcatatgcttaccgtaacttgaaag
tatttcgatttcttggctttatatatcttgtggaaaggacgaaacacc
gctctccccaccccaccttctgttttagagctagaaatagcaagttaa
aataaggctagtccgttatcaacttgaaaaagtggcaccgagtcggtg
cttttttgaattcgctagctaggtcttgaaaggagtgggaattggctc
cggtgcccgtcagtgggcagagcgcacatcgcccacagtccccgagaa
gttggggggaggggtcggcaattgatccggtgcctagagaaggtggcg
cggggtaaactgggaaagtgatgtcgtgtactggctccgcctttttcc
cgaggggggggagaaccgtatataagtgcagtagtcgccgtgaacgtt
ctttttcgcaacgggtttgccgccagaacacaggaccggtgccaccat
ggactataaggaccacgacggagactacaaggatcatgatattgatta
caaagacgatgacgataagatggccccaaagaagaagcggaaggtcgg
tatccacggagtcccagcagccgacaagaagtacagcatcggcctggc
catcggcaccaactctgtgggctgggccgtgatcaccgacgagtacaa
ggtgcccagcaagaaattcaaggtgctgggcaacaccgaccggcacag
catcaagaagaacctgatcggagccctgctgttcgacagcggcgaaac
agccgaggccacccggctgaagagaaccgccagaagaagatacaccag
acggaagaaccggatctgctatctgcaagagatcttcagcaacgagat
ggccaaggtggacgacagcttcttccacagactggaagagtccttcct
ggtggaagaggataagaagcacgagcggcaccccatcttcggcaacat
cgtggacgaggtggcctaccacgagaagtaccccaccatctaccacct
gagaaagaaactggtggacagcaccgacaaggccgacctgcggctgat
ctatctggccctggcccacatgatcaagttccggggccacttcctgat
cgagggcgacctgaaccccgacaacagcgacgtggacaagctgttcat
ccagctggtgcagacctacaaccagctgttcgaggaaaaccccatcaa
cgccagcggcgtggacgccaaggccatcctgtctgccagactgagcaa
gagcagacggctggaaaatctgatcgcccagctgcccggcgagaagaa
gaatggcctgttcggcaacctgattgccctgagcctgggcctgacccc
caacttcaagagcaacttcgacctggccgaggatgccaaactgcagct
gagcaaggacacctacgacgacgacctggacaacctgctggcccagat
cggcgaccagtacgccgacctgtttctggccgccaagaacctgtccga
cgccatcctgctgagcgacatcctgagagtgaacaccgagatcaccaa
ggcccccctgagcgcctctatgatcaagagatacgacgagcaccacca
ggacctgaccctgctgaaagctctcgtgcggcagcagctgcctgagaa
gtacaaagagattttcttcgaccagagcaagaacggctacgccggcta
cattgacggcggagccagccaggaagagttctacaagttcatcaagcc
catcctggaaaagatggacggcaccgaggaactgctcgtgaagctgaa
cagagaggacctgctgcggaagcagcggaccttcgacaacggcagcat
cccccaccagatccacctgggagagctgcacgccattctgcggcggca
ggaagatttttacccattcctgaaggacaaccgggaaaagatcgagaa
gatcctgaccttccgcatcccctactacgtgggccctctggccagggg
aaacagcagattcgcctggatgaccagaaagagcgaggaaaccatcac
cccctggaacttcgaggaagtggtggacaagggcgcttccgcccagag
cttcatcgagcggatgaccaacttcgataagaacctgcccaacgagaa
ggtgctgcccaagcacagcctgctgtacgagtacttcaccgtgtataa
cgagctgaccaaagtgaaatacgtgaccgagggaatgagaaagcccgc
cttcctgagcggcgagcagaaaaaggccatcgtggacctgctgttcaa
gaccaaccggaaagtgaccgtgaagcagctgaaagaggactacttcaa
gaaaatcgagtgcttcgactccgtggaaatctccggcgtggaagatcg
gttcaacgcctccctgggcacataccacgatctgctgaaaattatcaa
ggacaaggacttcctggacaatgaggaaaacgaggacattctggaaga
tatcgtgctgaccctgacactgtttgaggacagagagatgatcgagga
acggctgaaaacctatgcccacctgttcgacgacaaagtgatgaagca
gctgaagcggcggagatacaccggctggggcaggctgagccggaagct
gatcaacggcatccgggacaagcagtccggcaagacaatcctggattt
cctgaagtccgacggcttcgccaacagaaacttcatgcagctgatcca
cgacgacagcctgacctttaaagaggacatccagaaagcccaggtgtc
cggccagggcgatagcctgcacgagcacattgccaatctggccggcag
ccccgccattaagaagggcatcctgcagacagtgaaggtggtggacga
gctcgtgaaagtgatgggccggcacaagcccgagaacatcgtgatcga
aatggccagagagaaccagaccacccagaagggacagaagaacagccg
cgagagaatgaagcggatcgaagagggcatcaaagagctgggcagcca
gatcctgaaagaacaccccgtggaaaacacccagctgcagaacgagaa
gctgtacctgtactacctgcagaatggggggatatgtacgtggaccag
gaactggacatcaaccggctgtccgactacgatgtggacgctatcgtg
cctcagagctttctgaaggacgactccatcgacaacaaggtgctgacc
agaagcgacaagaaccggggcaagagcgacaacgtgccctccgaagag
gtcgtgaagaagatgaagaactactggcggcagctgctgaacgccaag
ctgattacccagagaaagttcgacaatctgaccaaggccgagagaggc
ggcctgagcgaactggataaggccggcttcatcaagagacagctggtg
gaaacccggcagatcacaaagcacgtggcacagatactagattcccga
atgaatacgaaatacgacgagaacgataagctgattcgggaagtcaaa
gtaatcactttaaagtcaaaattggtgtcggacttcagaaaggatttt
caattctataaagttagggagataaataactaccaccatgcgcacgac
gcttatcttaatgccgtcgtagggaccgcactcattaagaaatacccg
aagctagaaagtgagtttgtgtatggtgattacaaagtttatgacgtc
cgtaagatgatcgcgaaaagcgaacaggagataggcaaggctacagcc
aaatacttcttttattctaacattatgaatttctttaagacggaaatc
actctggcaaacggagagatacgcaaacgacctttaattgaaaccaat
ggggagacaggtgaaatcgtatgggataagggccgggacttcgcgacg
gtgagaaaagttttgtccatgccccaagtcaacatagtaaagaaaact
gaggtgcagaccggagggttttcaaaggaatcgattcttccaaaaagg
aatagtgataagctcatcgctcgtaaaaaggactgggacccgaaaaag
tacggtggcttcgtgagccctacagttgcctattctgtcctagtagtg
gcaaaagttgagaagggaaaatccaagaaactgaagtcagtcaaagaa
ttattggggataacgattatggagcgctcgtcttttgaaaagaacccc
atcgacttccttgaggcgaaaggttacaaggaagtaaaaaaggatctc
ataattaaactaccaaagtatagtctgtttgagttagaaaatggccga
aaacggatgttggctagcgccagagagcttcaaaaggggaacgaactc
gcactaccgtctaaatacgtgaatttcctgtatttagcgtcccattac
gagaagttgaaaggttcacctgaagataacgaacagaagcaacttttt
gttgagcagcacaaacattatctcgacgaaatcatagagcaaatttcg
gaattcagtaagagagtcatcctagctgatgccaatctggacaaagta
ttaagcgcatacaacaagcacagggataaacccatacgtgagcaggcg
gaaaatattatccatttgtttactcttaccaacctcggcgctccagcc
gcattcaagtattttgacacaacgatagatcgcaaagagtacagatct
accaaggaggtgctagacgcgacactgattcaccaatccatcacggga
ttatatgaaactcggatagatttgtcacagcttgggggtgacggatcc
cccaagaagaagaggaaagtcctcgagggcggaggcgggagcggatcc
ccctcccggctccagatgttcttcgctaataaccacgaccaggaattt
gaccctccaaaggtttacccacctgtcccagctgagaagaggaagccc
atccgggtgctgtctctctttgatggaatcgctacagggctcctggtg
ctgaaggacttgggcattcaggtggaccgctacattgcctcggaggtg
tgtgaggactccatcacggtgggcatggtgcggcaccaggggaagatc
atgtacgtcggggacgtccgcagcgtcacacagaagcatatccaggag
tggggcccattcgatctggtgattgggggcagtccctgcaatgacctc
tccatcgtcaaccctgctcgcaagggcctctacgagggcactggccgg
ctcttctttgagttctaccgcctcctgcatgatgcgcggcccaaggag
ggagatgatcgccccttcttctggctctttgagaatgtggtggccatg
ggcgttagtgacaagagggacatctcgcgatttctcgagtccaaccct
gtgatgattgatgccaaagaagtgtcagctgcacacagggcccgctac
ttctggggtaaccttcccggtatgaacaggccgttggcatccactgtg
aatgataagctggagctgcaggagtgtctggagcatggcaggatagcc
aagttcagcaaagtgaggaccattactacgaggtcaaactccataaag
cagggcaaagaccagcattttcctgtgttcatgaatgagaaagaggac
atcttatggtgcactgaaatggaaagggtatttggtttcccagtccac
tatactgacgtgtccaacatgagccgcttggcgaggcagagactgctg
ggccggtcatggagcgtgccagtcatccgccacctcttcgctccgctg
aaggagtattttgcgtgtgtgtccggccggcccggatccggcgcaaca
aacttctctctgctgaaacaagccggagatgtcgaagagaatcctgga
ccgaccgagtacaagcccacggtgcgcctcgccacccgcgacgacgtc
cccagggccgtacgcaccctcgccgccgcgttcgccgactaccccgcc
acgcgccacaccgtcgatccggaccgccacatcgagcgggtcaccgag
ctgcaagaactcttcctcacgcgcgtcgggctcgacatcggcaaggtg
tgggtcgcggacgacggcgccgcggtggcggtctggaccacgccggag
agcgtcgaagcgggggcggtgttcgccgagatcggcccgcgcatggcc
gagttgagcggttcccggctggccgcgcagcaacagatggaaggcctc
ctggcgccgcaccggcccaaggagcccgcgtggttcctggccaccgtc
ggagtctcgcccgaccaccagggcaagggtctgggcagcgccgtcgtg
ctccccggagtggaggcggccgagcgcgccggggtgcccgccttcctg
gagacctccgcgccccgcaacctccccttctacgagcggctcggcttc
accgtcaccgccgacgtcgaggtgcccgaaggaccgcgcacctggtgc
atgacccgcaagcccggtgcctgaacgcgttaagtcgacaatcaacct
ctggattacaaaatttgtgaaagattgactggtattcttaactatgtt
gctccttttacgctatgtggatacgctgctttaatgcctttgtatcat
gctattgcttcccgtatggctttcattttctcctccttgtataaatcc
tggttgctgtctctttatgaggagttgtggcccgttgtcaggcaacgt
ggcgtggtgtgcactgtgtttgctgacgcaacccccactggttggggc
attgccaccacctgtcagctcctttccgggactttcgctttccccctc
cctattgccacggcggaactcatcgccgcctgccttgcccgctgctgg
acaggggctcggctgttgggcactgacaattccgtggtgttgtcgggg
aaatcatcgtcctttccttggctgctcgcctgtgttgccacctggatt
ctgcgcgggacgtccttctgctacgtcccttcggccctcaatccagcg
gaccttccttcccgcggcctgctgccggctctgcggcctcttccgcgt
cttcgccttcgccctcagacgagtcggatctccctttgggccgcctcc
ccgcgtcgactttaagaccaatgacttacaaggcagctgtagatctta
gccactttttaaaagaaaaggggggactggaagggctaattcactccc
aacgaagacaagatctgctttttgcttgtactgggtctctctggttag
accagatctgagcctgggagctctctggctaactagggaacccactgc
ttaagcctcaataaagcttgccttgagtgcttcaagtagtgtgtgccc
gtctgttgtgtgactctggtaactagagatccctcagacccttttagt
cagtgtggaaaatctctagcagggcccgtttaaacccgctgatcagcc
tcgactgtgccttctagttgccagccatctgttgtttgcccctccccc
gtgccttccttgaccctggaaggtgccactcccactgtcctttcctaa
taaaatgaggaaattgcatcgcattgtctgagtaggtgtcattctatt
ctggggggggggtggggcaggacagcaagggggaggattgggaagaca
atagcaggcatgctggggatgcggtgggctctatggcttctgaggcgg
aaagaaccagctggggctctagggggtatccccacgcgccctgtagcg
gcgcattaagcgcggcgggtgtggtggttacgcgcagcgtgaccgcta
cacttgccagcgccctagcgcccgctcctttcgctttcttcccttcct
ttctcgccacgttcgccggctttccccgtcaagctctaaatcgggggc
tccctttagggttccgatttagtgctttacggcacctcgaccccaaaa
aacttgattagggtgatggttcacgtagtgggccatcgccctgataga
cggtttttcgccctttgacgttggagtccacgttctttaatagtggac
tcttgttccaaactggaacaacactcaaccctatctcggtctattctt
ttgatttataagggattttgccgatttcggcctattggttaaaaaatg
agctgatttaacaaaaatttaacgcgaattaattctgtggaatgtgtg
tcagttagggtgtggaaagtccccaggctccccagcaggcagaagtat
gcaaagcatgcatctcaattagtcagcaaccaggtgtggaaagtcccc
aggctccccagcaggcagaagtatgcaaagcatgcatctcaattagtc
agcaaccatagtcccgcccctaactccgcccatcccgcccctaactcc
gcccagttccgcccattctccgccccatggctgactaattttttttat
ttatgcagaggccgaggccgcctctgcctctgagctattccagaagta
gtgaggaggcttttttggaggcctaggcttttgcaaaaagctccctac
cgtcgacctctagctagagcttggcgtaatcatggtcatagctgtttc
ctgtgtgaaattgttatccgctcacaattccacacaacatacgagccg
gaagcataaagtgtaaagcctggggtgcctaatgagtgagctaactca
cattaattgcgttgcgctcactgcccgctttccagtcgggaaacctgt
cgtgccagctgcattaatgaatcggccaacgcgcggggagaggcggtt
tgcgtattgggcgctcttccgcttcctcgctcactgactcgctgcgct
cggtcgttcggctgcggcgagcggtatcagctcactcaaaggcggtaa
tacggttatccacagaatcaggggataacgcaggaaagaacatgtgag
caaaaggccagcaaaaggccaggaaccgtaaaaaggccgcgttgctgg
cgtttttccataggctccgcccccctgacgagcatcacaaaaatcgac
gctcaagtcagaggtggcgaaacccgacaggactataaagataccagg
cgtttccccctggaagctccctcgtgcgctctcctgttccgaccctgc
cgcttaccggatacctgtccgcctttctcccttcgggaagcgtggcgc
tttctcatagctcacgctgtaggtatctcagttcggtgtaggtcgttc
gctccaagctgggctgtgtgcacgaaccccccgttcagcccgaccgct
gcgccttatccggtaactatcgtcttgagtccaacccggtaagacacg
acttatcgccactggcagcagccactggtaacaggattagcagagcga
ggtatgtaggcggtgctacagagttcttgaagtggtggcctaactacg
gctacactagaagaacagtatttggtatctgcgctctgctgaagccag
ttaccttcggaaaaagagttggtagctcttgatccggcaaacaaacca
ccgctggtagcggtggtttttttgtttgcaagcagcagattacgcgca
gaaaaaaaggatctcaagaagatcctttgatcttttctacggggtctg
acgctcagtggaacgaaaactcacgttaagggattttggtcatgagat
tatcaaaaaggatcttcacctagatccttttaaattaaaaatgaagtt
ttaaatcaatctaaagtatatatgagtaaacttggtctgacagttacc
aatgcttaatcagtgaggcacctatctcagcgatctgtctatttcgtt
catccatagttgcctgactccccgtcgtgtagataactacgatacggg
agggcttaccatctggccccagtgctgcaatgataccgcgagacccac
gctcaccggctccagatttatcagcaataaaccagccagccggaaggg
ccgagcgcagaagtggtcctgcaactttatccgcctccatccagtcta
ttaattgttgccgggaagctagagtaagtagttcgccagttaatagtt
tgcgcaacgttgttgccattgctacaggcatcgtggtgtcacgctcgt
cgtttggtatggcttcattcagctccggttcccaacgatcaaggcgag
ttacatgatcccccatgttgtgcaaaaaagcggttagctccttcggtc
ctccgatcgttgtcagaagtaagttggccgcagtgttatcactcatgg
ttatggcagcactgcataattctcttactgtcatgccatccgtaagat
gcttttctgtgactggtgagtactcaaccaagtcattctgagaatagt
gtatgcggcgaccgagttgctcttgcccggcgtcaatacgggataata
ccgcgccacatagcagaactttaaaagtgctcatcattggaaaacgtt
cttcggggcgaaaactctcaaggatcttaccgctgttgagatccagtt
cgatgtaacccactcgtgcacccaactgatcttcagcatcttttactt
tcaccagcgtttctgggtgagcaaaaacaggaaggcaaaatgccgcaa
aaaagggaataagggcgacacggaaatgttgaatactcatactcttcc
tttttcaatattattgaagcatttatcagggttattgtctcatgagcg
gatacatatttgaatgtatttagaaaaataaacaaataggggttccgc
gcacatttccccgaaaagtgccacctgac.

In an aspect, a disclosed pBK1108 plasmid can comprise the sequence set forth below or a sequence having 70%, 75%, 80%, 85%, 90%, 95%, or more identity to the sequence set forth

(SEQ ID NO: 46)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggtggagtatttacggtaaactgcccacttggcagtacatcaag
tgtatcatatgccaagtacgccccctattgacgtcaatgacggtaaat
ggcccgcctggcattatgcccagtacatgaccttatgggactttccta
cttggcagtacatctacgtattagtcatcgctattaccatggtgatgc
ggttttggcagtacatcaatgggcgtggatagcggtttgactcacggg
gatttccaagtctccaccccattgacgtcaatgggagtttgttttggc
accaaaatcaacgggactttccaaaatgtcgtaacaactccgccccat
tgacgcaaatgggcggtaggcgtgtacggtgggaggtctatataagca
gcgcgttttgcctgtactgggtctctctggttagaccagatctgagcc
tgggagctctctggctaactagggaacccactgcttaagcctcaataa
agcttgccttgagtgcttcaagtagtgtgtgcccgtctgttgtgtgac
tctggtaactagagatccctcagacccttttagtcagtgtggaaaatc
tctagcagtggcgcccgaacagggacttgaaagcgaaagggaaaccag
aggagctctctcgacgcaggactcggcttgctgaagcgcgcacggcaa
gaggcgaggggcggcgactggtgagtacgccaaaaattttgactagcg
gaggctagaaggagagagatgggtgcgagagcgtcagtattaagcggg
ggagaattagatcgcgatgggaaaaaattcggttaaggccagggggaa
agaaaaaatataaattaaaacatatagtatgggcaagcagggagctag
aacgattcgcagttaatcctggcctgttagaaacatcagaaggctgta
gacaaatactgggacagctacaaccatcccttcagacaggatcagaag
aacttagatcattatataatacagtagcaaccctctattgtgtgcatc
aaaggatagagataaaagacaccaaggaagctttagacaagatagagg
aagagcaaaacaaaagtaagaccaccgcacagcaagcggccgctgatc
ttcagacctggaggaggagatatgagggacaattggagaagtgaatta
tataaatataaagtagtaaaaattgaaccattaggagtagcacccacc
aaggcaaagagaagagtggtgcagagagaaaaaagagcagtgggaata
ggagctttgttccttgggttcttgggagcagcaggaagcactatgggc
gcagcgtcaatgacgctgacggtacaggccagacaattattgtctggt
atagtgcagcagcagaacaatttgctgagggctattgaggcgcaacag
catctgttgcaactcacagtctggggcatcaagcagctccaggcaaga
atcctggctgtggaaagatacctaaaggatcaacagctcctggggatt
tggggttgctctggaaaactcatttgcaccactgctgtgccttggaat
gctagttggagtaataaatctctggaacagatttggaatcacacgacc
tggatggagtgggacagagaaattaacaattacacaagcttaatacac
tccttaattgaagaatcgcaaaaccagcaagaaaagaatgaacaagaa
ttattggaattagataaatgggcaagtttgtggaattggtttaacata
acaaattggctgtggtatataaaattattcataatgatagtaggaggc
ttggtaggtttaagaatagtttttgctgtactttctatagtgaataga
gttaggcagggatattcaccattatcgtttcagacccacctcccaacc
ccgaggggacccgacaggcccgaaggaatagaagaagaaggtggagag
agagacagagacagatccattcgattagtgaacggatcggcactgcgt
gcgccaattctgcagacaaatggcagtattcatccacaattttaaaag
aaaaggggggattggggggtacagtgcaggggaaagaatagtagacat
aatagcaacagacatacaaactaaagaattacaaaaacaaattacaaa
aattcaaaattttcgggtttattacagggacagcagagatccagtttg
gttaattaatggggggacgttaacggggcggaacggtaccgagggcct
atttcccatgattccttcatatttgcatatacgatacaaggctgttag
agagataattagaattaatttgactgtaaacacaaagatattagtaca
aaatacgtgacgtagaaagtaataatttcttgggtagtttgcagtttt
aaaattatgttttaaaatggactatcatatgcttaccgtaacttgaaa
gtatttcgatttcttggctttatatatcttgtggaaaggacgaaacac
cgtgtgaagggagaatgaggaagttttagagctagaaatagcaagtta
aaataaggctagtccgttatcaacttgaaaaagtggcaccgagtcggt
gcttttttgaattcgctagctaggtcttgaaaggagtgggaattggct
ccggtgcccgtcagtgggcagagcgcacatcgcccacagtccccgaga
agttggggggaggggtcggcaattgatccggtgcctagagaaggtggc
gcggggtaaactgggaaagtgatgtcgtgtactggctccgcctttttc
ccgaggggggggagaaccgtatataagtgcagtagtcgccgtgaacgt
tctttttcgcaacgggtttgccgccagaacacaggaccggtgccacca
tggactataaggaccacgacggagactacaaggatcatgatattgatt
acaaagacgatgacgataagatggccccaaagaagaagcggaaggtcg
gtatccacggagtcccagcagccgacaagaagtacagcatcggcctgg
ccatcggcaccaactctgtgggctgggccgtgatcaccgacgagtaca
aggtgcccagcaagaaattcaaggtgctgggcaacaccgaccggcaca
gcatcaagaagaacctgatcggagccctgctgttcgacagcggcgaaa
cagccgaggccacccggctgaagagaaccgccagaagaagatacacca
gacggaagaaccggatctgctatctgcaagagatcttcagcaacgaga
tggccaaggtggacgacagcttcttccacagactggaagagtccttcc
tggtggaagaggataagaagcacgagcggcaccccatcttcggcaaca
tcgtggacgaggtggcctaccacgagaagtaccccaccatctaccacc
tgagaaagaaactggtggacagcaccgacaaggccgacctgcggctga
tctatctggccctggcccacatgatcaagttccggggccacttcctga
tcgagggcgacctgaaccccgacaacagcgacgtggacaagctgttca
tccagctggtgcagacctacaaccagctgttcgaggaaaaccccatca
acgccagcggcgtggacgccaaggccatcctgtctgccagactgagca
agagcagacggctggaaaatctgatcgcccagctgcccggcgagaaga
agaatggcctgttcggcaacctgattgccctgagcctgggcctgaccc
ccaacttcaagagcaacttcgacctggccgaggatgccaaactgcagc
tgagcaaggacacctacgacgacgacctggacaacctgctggcccaga
tcggcgaccagtacgccgacctgtttctggccgccaagaacctgtccg
acgccatcctgctgagcgacatcctgagagtgaacaccgagatcacca
aggcccccctgagcgcctctatgatcaagagatacgacgagcaccacc
aggacctgaccctgctgaaagctctcgtgcggcagcagctgcctgaga
agtacaaagagattttcttcgaccagagcaagaacggctacgccggct
acattgacggcggagccagccaggaagagttctacaagttcatcaagc
ccatcctggaaaagatggacggcaccgaggaactgctcgtgaagctga
acagagaggacctgctgcggaagcagcggaccttcgacaacggcagca
tcccccaccagatccacctgggagagctgcacgccattctgcggcggc
aggaagatttttacccattcctgaaggacaaccgggaaaagatcgaga
agatcctgaccttccgcatcccctactacgtgggccctctggccaggg
gaaacagcagattcgcctggatgaccagaaagagcgaggaaaccatca
ccccctggaacttcgaggaagtggtggacaagggcgcttccgcccaga
gcttcatcgagcggatgaccaacttcgataagaacctgcccaacgaga
aggtgctgcccaagcacagcctgctgtacgagtacttcaccgtgtata
acgagctgaccaaagtgaaatacgtgaccgagggaatgagaaagcccg
ccttcctgagcggcgagcagaaaaaggccatcgtggacctgctgttca
agaccaaccggaaagtgaccgtgaagcagctgaaagaggactacttca
agaaaatcgagtgcttcgactccgtggaaatctccggcgtggaagatc
ggttcaacgcctccctgggcacataccacgatctgctgaaaattatca
aggacaaggacttcctggacaatgaggaaaacgaggacattctggaag
atatcgtgctgaccctgacactgtttgaggacagagagatgatcgagg
aacggctgaaaacctatgcccacctgttcgacgacaaagtgatgaagc
agctgaagcggcggagatacaccggctggggcaggctgagccggaagc
tgatcaacggcatccgggacaagcagtccggcaagacaatcctggatt
tcctgaagtccgacggcttcgccaacagaaacttcatgcagctgatcc
acgacgacagcctgacctttaaagaggacatccagaaagcccaggtgt
ccggccagggcgatagcctgcacgagcacattgccaatctggccggca
gccccgccattaagaagggcatcctgcagacagtgaaggtggtggacg
agctcgtgaaagtgatgggccggcacaagcccgagaacatcgtgatcg
aaatggccagagagaaccagaccacccagaagggacagaagaacagcc
gcgagagaatgaagcggatcgaagagggcatcaaagagctgggcagcc
agatcctgaaagaacaccccgtggaaaacacccagctgcagaacgaga
agctgtacctgtactacctgcagaatggggggatatgtacgtggacca
ggaactggacatcaaccggctgtccgactacgatgtggacgctatcgt
gcctcagagctttctgaaggacgactccatcgacaacaaggtgctgac
cagaagcgacaagaaccggggcaagagcgacaacgtgccctccgaaga
ggtcgtgaagaagatgaagaactactggcggcagctgctgaacgccaa
gctgattacccagagaaagttcgacaatctgaccaaggccgagagagg
cggcctgagcgaactggataaggccggcttcatcaagagacagctggt
ggaaacccggcagatcacaaagcacgtggcacagatactagattcccg
aatgaatacgaaatacgacgagaacgataagctgattcgggaagtcaa
agtaatcactttaaagtcaaaattggtgtcggacttcagaaaggattt
tcaattctataaagttagggagataaataactaccaccatgcgcacga
cgcttatcttaatgccgtcgtagggaccgcactcattaagaaataccc
gaagctagaaagtgagtttgtgtatggtgattacaaagtttatgacgt
ccgtaagatgatcgcgaaaagcgaacaggagataggcaaggctacagc
caaatacttcttttattctaacattatgaatttctttaagacggaaat
cactctggcaaacggagagatacgcaaacgacctttaattgaaaccaa
tggggagacaggtgaaatcgtatgggataagggccgggacttcgcgac
ggtgagaaaagttttgtccatgccccaagtcaacatagtaaagaaaac
tgaggtgcagaccggagggttttcaaaggaatcgattcttccaaaaag
gaatagtgataagctcatcgctcgtaaaaaggactgggacccgaaaaa
gtacggtggcttcgtgagccctacagttgcctattctgtcctagtagt
ggcaaaagttgagaagggaaaatccaagaaactgaagtcagtcaaaga
attattggggataacgattatggagcgctcgtcttttgaaaagaaccc
catcgacttccttgaggcgaaaggttacaaggaagtaaaaaaggatct
cataattaaactaccaaagtatagtctgtttgagttagaaaatggccg
aaaacggatgttggctagcgccagagagcttcaaaaggggaacgaact
cgcactaccgtctaaatacgtgaatttcctgtatttagcgtcccatta
cgagaagttgaaaggttcacctgaagataacgaacagaagcaactttt
tgttgagcagcacaaacattatctcgacgaaatcatagagcaaatttc
ggaattcagtaagagagtcatcctagctgatgccaatctggacaaagt
attaagcgcatacaacaagcacagggataaacccatacgtgagcaggc
ggaaaatattatccatttgtttactcttaccaacctcggcgctccagc
cgcattcaagtattttgacacaacgatagatcgcaaagagtacagatc
taccaaggaggtgctagacgcgacactgattcaccaatccatcacggg
attatatgaaactcggatagatttgtcacagcttgggggtgacggatc
ccccaagaagaagaggaaagtcctcgagggcggaggcgggagcggatc
cccctcccggctccagatgttcttcgctaataaccacgaccaggaatt
tgaccctccaaaggtttacccacctgtcccagctgagaagaggaagcc
catccgggtgctgtctctctttgatggaatcgctacagggctcctggt
gctgaaggacttgggcattcaggtggaccgctacattgcctcggaggt
gtgtgaggactccatcacggtgggcatggtgcggcaccaggggaagat
catgtacgtcggggacgtccgcagcgtcacacagaagcatatccagga
gtggggcccattcgatctggtgattgggggcagtccctgcaatgacct
ctccatcgtcaaccctgctcgcaagggcctctacgagggcactggccg
gctcttctttgagttctaccgcctcctgcatgatgcgcggcccaagga
gggagatgatcgccccttcttctggctctttgagaatgtggtggccat
gggcgttagtgacaagagggacatctcgcgatttctcgagtccaaccc
tgtgatgattgatgccaaagaagtgtcagctgcacacagggcccgcta
cttctggggtaaccttcccggtatgaacaggccgttggcatccactgt
gaatgataagctggagctgcaggagtgtctggagcatggcaggatagc
caagttcagcaaagtgaggaccattactacgaggtcaaactccataaa
gcagggcaaagaccagcattttcctgtgttcatgaatgagaaagagga
catcttatggtgcactgaaatggaaagggtatttggtttcccagtcca
ctatactgacgtgtccaacatgagccgcttggcgaggcagagactgct
gggccggtcatggagcgtgccagtcatccgccacctcttcgctccgct
gaaggagtattttgcgtgtgtgtccggccggcccggatccggcgcaac
aaacttctctctgctgaaacaagccggagatgtcgaagagaatcctgg
accgaccgagtacaagcccacggtgcgcctcgccacccgcgacgacgt
ccccagggccgtacgcaccctcgccgccgcgttcgccgactaccccgc
cacgcgccacaccgtcgatccggaccgccacatcgagcgggtcaccga
gctgcaagaactcttcctcacgcgcgtcgggctcgacatcggcaaggt
gtgggtcgcggacgacggcgccgcggtggcggtctggaccacgccgga
gagcgtcgaagcgggggcggtgttcgccgagatcggcccgcgcatggc
cgagttgagcggttcccggctggccgcgcagcaacagatggaaggcct
cctggcgccgcaccggcccaaggagcccgcgtggttcctggccaccgt
cggagtctcgcccgaccaccagggcaagggtctgggcagcgccgtcgt
gctccccggagtggaggcggccgagcgcgccggggtgcccgccttcct
ggagacctccgcgccccgcaacctccccttctacgagcggctcggctt
caccgtcaccgccgacgtcgaggtgcccgaaggaccgcgcacctggtg
catgacccgcaagcccggtgcctgaacgcgttaagtcgacaatcaacc
tctggattacaaaatttgtgaaagattgactggtattcttaactatgt
tgctccttttacgctatgtggatacgctgctttaatgcctttgtatca
tgctattgcttcccgtatggctttcattttctcctccttgtataaatc
ctggttgctgtctctttatgaggagttgtggcccgttgtcaggcaacg
tggcgtggtgtgcactgtgtttgctgacgcaacccccactggttgggg
cattgccaccacctgtcagctcctttccgggactttcgctttccccct
ccctattgccacggcggaactcatcgccgcctgccttgcccgctgctg
gacaggggctcggctgttgggcactgacaattccgtggtgttgtcggg
gaaatcatcgtcctttccttggctgctcgcctgtgttgccacctggat
tctgcgcgggacgtccttctgctacgtcccttcggccctcaatccagc
ggaccttccttcccgcggcctgctgccggctctgcggcctcttccgcg
tcttcgccttcgccctcagacgagtcggatctccctttgggccgcctc
cccgcgtcgactttaagaccaatgacttacaaggcagctgtagatctt
agccactttttaaaagaaaaggggggactggaagggctaattcactcc
caacgaagacaagatctgctttttgcttgtactgggtctctctggtta
gaccagatctgagcctgggagctctctggctaactagggaacccactg
cttaagcctcaataaagcttgccttgagtgcttcaagtagtgtgtgcc
cgtctgttgtgtgactctggtaactagagatccctcagacccttttag
tcagtgtggaaaatctctagcagggcccgtttaaacccgctgatcagc
ctcgactgtgccttctagttgccagccatctgttgtttgcccctcccc
cgtgccttccttgaccctggaaggtgccactcccactgtcctttccta
ataaaatgaggaaattgcatcgcattgtctgagtaggtgtcattctat
tctggggggtggggggggcaggacagcaagggggaggattgggaagac
aatagcaggcatgctggggatgcggtgggctctatggcttctgaggcg
gaaagaaccagctggggctctagggggtatccccacgcgccctgtagc
ggcgcattaagcgcggcgggtgtggtggttacgcgcagcgtgaccgct
acacttgccagcgccctagcgcccgctcctttcgctttcttcccttcc
tttctcgccacgttcgccggctttccccgtcaagctctaaatcggggg
ctccctttagggttccgatttagtgctttacggcacctcgaccccaaa
aaacttgattagggtgatggttcacgtagtgggccatcgccctgatag
acggtttttcgccctttgacgttggagtccacgttctttaatagtgga
ctcttgttccaaactggaacaacactcaaccctatctcggtctattct
tttgatttataagggattttgccgatttcggcctattggttaaaaaat
gagctgatttaacaaaaatttaacgcgaattaattctgtggaatgtgt
gtcagttagggtgtggaaagtccccaggctccccagcaggcagaagta
tgcaaagcatgcatctcaattagtcagcaaccaggtgtggaaagtccc
caggctccccagcaggcagaagtatgcaaagcatgcatctcaattagt
cagcaaccatagtcccgcccctaactccgcccatcccgcccctaactc
cgcccagttccgcccattctccgccccatggctgactaatttttttta
tttatgcagaggccgaggccgcctctgcctctgagctattccagaagt
agtgaggaggcttttttggaggcctaggcttttgcaaaaagctcccta
ccgtcgacctctagctagagcttggcgtaatcatggtcatagctgttt
cctgtgtgaaattgttatccgctcacaattccacacaacatacgagcc
ggaagcataaagtgtaaagcctggggtgcctaatgagtgagctaactc
acattaattgcgttgcgctcactgcccgctttccagtcgggaaacctg
tcgtgccagctgcattaatgaatcggccaacgcgcggggagaggcggt
ttgcgtattgggcgctcttccgcttcctcgctcactgactcgctgcgc
tcggtcgttcggctgcggcgagcggtatcagctcactcaaaggcggta
atacggttatccacagaatcaggggataacgcaggaaagaacatgtga
gcaaaaggccagcaaaaggccaggaaccgtaaaaaggccgcgttgctg
gcgtttttccataggctccgcccccctgacgagcatcacaaaaatcga
cgctcaagtcagaggtggcgaaacccgacaggactataaagataccag
gcgtttccccctggaagctccctcgtgcgctctcctgttccgaccctg
ccgcttaccggatacctgtccgcctttctcccttcgggaagcgtggcg
ctttctcatagctcacgctgtaggtatctcagttcggtgtaggtcgtt
cgctccaagctgggctgtgtgcacgaaccccccgttcagcccgaccgc
tgcgccttatccggtaactatcgtcttgagtccaacccggtaagacac
gacttatcgccactggcagcagccactggtaacaggattagcagagcg
aggtatgtaggcggtgctacagagttcttgaagtggtggcctaactac
ggctacactagaagaacagtatttggtatctgcgctctgctgaagcca
gttaccttcggaaaaagagttggtagctcttgatccggcaaacaaacc
accgctggtagcggtggtttttttgtttgcaagcagcagattacgcgc
agaaaaaaaggatctcaagaagatcctttgatcttttctacggggtct
gacgctcagtggaacgaaaactcacgttaagggattttggtcatgaga
ttatcaaaaaggatcttcacctagatccttttaaattaaaaatgaagt
tttaaatcaatctaaagtatatatgagtaaacttggtctgacagttac
caatgcttaatcagtgaggcacctatctcagcgatctgtctatttcgt
tcatccatagttgcctgactccccgtcgtgtagataactacgatacgg
gagggcttaccatctggccccagtgctgcaatgataccgcgagaccca
cgctcaccggctccagatttatcagcaataaaccagccagccggaagg
gccgagcgcagaagtggtcctgcaactttatccgcctccatccagtct
attaattgttgccgggaagctagagtaagtagttcgccagttaatagt
ttgcgcaacgttgttgccattgctacaggcatcgtggtgtcacgctcg
tcgtttggtatggcttcattcagctccggttcccaacgatcaaggcga
gttacatgatcccccatgttgtgcaaaaaagcggttagctccttcggt
cctccgatcgttgtcagaagtaagttggccgcagtgttatcactcatg
gttatggcagcactgcataattctcttactgtcatgccatccgtaaga
tgcttttctgtgactggtgagtactcaaccaagtcattctgagaatag
tgtatgcggcgaccgagttgctcttgcccggcgtcaatacgggataat
accgcgccacatagcagaactttaaaagtgctcatcattggaaaacgt
tcttcggggcgaaaactctcaaggatcttaccgctgttgagatccagt
tcgatgtaacccactcgtgcacccaactgatcttcagcatcttttact
ttcaccagcgtttctgggtgagcaaaaacaggaaggcaaaatgccgca
aaaaagggaataagggcgacacggaaatgttgaatactcatactcttc
ctttttcaatattattgaagcatttatcagggttattgtctcatgagc
ggatacatatttgaatgtatttagaaaaataaacaaataggggttccg
cgcacatttccccgaaaagtgccacctgac.

In an aspect, a disclosed pBK1109 plasmid can comprise the sequence set forth below or a sequence having 70%, 75%, 80%, 85%, 90%, 95%, or more identity to the sequence set forth

(SEQ ID NO: 47)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggtggagtatttacggtaaactgcccacttggcagtacatcaag
tgtatcatatgccaagtacgccccctattgacgtcaatgacggtaaat
ggcccgcctggcattatgcccagtacatgaccttatgggactttccta
cttggcagtacatctacgtattagtcatcgctattaccatggtgatgc
ggttttggcagtacatcaatgggcgtggatagcggtttgactcacggg
gatttccaagtctccaccccattgacgtcaatgggagtttgttttggc
accaaaatcaacgggactttccaaaatgtcgtaacaactccgccccat
tgacgcaaatgggcggtaggcgtgtacgggggaggtctatataagcag
cgcgttttgcctgtactgggtctctctggttagaccagatctgagcct
gggagctctctggctaactagggaacccactgcttaagcctcaataaa
gcttgccttgagtgcttcaagtagtgtgtgcccgtctgttgtgtgact
ctggtaactagagatccctcagacccttttagtcagtgtggaaaatct
ctagcagtggcgcccgaacagggacttgaaagcgaaagggaaaccaga
ggagctctctcgacgcaggactcggcttgctgaagcgcgcacggcaag
aggcgaggggcggcgactggtgagtacgccaaaaattttgactagcgg
aggctagaaggagagagatgggtgcgagagcgtcagtattaagcgggg
gagaattagatcgcgatgggaaaaaattcggttaaggccagggggaaa
gaaaaaatataaattaaaacatatagtatgggcaagcagggagctaga
acgattcgcagttaatcctggcctgttagaaacatcagaaggctgtag
acaaatactgggacagctacaaccatcccttcagacaggatcagaaga
acttagatcattatataatacagtagcaaccctctattgtgtgcatca
aaggatagagataaaagacaccaaggaagctttagacaagatagagga
agagcaaaacaaaagtaagaccaccgcacagcaagcggccgctgatct
tcagacctggaggaggagatatgagggacaattggagaagtgaattat
ataaatataaagtagtaaaaattgaaccattaggagtagcacccacca
aggcaaagagaagagtggtgcagagagaaaaaagagcagtgggaatag
gagctttgttccttgggttcttgggagcagcaggaagcactatgggcg
cagcgtcaatgacgctgacggtacaggccagacaattattgtctggta
tagtgcagcagcagaacaatttgctgagggctattgaggcgcaacagc
atctgttgcaactcacagtctggggcatcaagcagctccaggcaagaa
tcctggctgtggaaagatacctaaaggatcaacagctcctggggattt
ggggttgctctggaaaactcatttgcaccactgctgtgccttggaatg
ctagttggagtaataaatctctggaacagatttggaatcacacgacct
ggatggagtgggacagagaaattaacaattacacaagcttaatacact
ccttaattgaagaatcgcaaaaccagcaagaaaagaatgaacaagaat
tattggaattagataaatgggcaagtttgtggaattggtttaacataa
caaattggctgtggtatataaaattattcataatgatagtaggaggct
tggtaggtttaagaatagtttttgctgtactttctatagtgaatagag
ttaggcagggatattcaccattatcgtttcagacccacctcccaaccc
cgaggggacccgacaggcccgaaggaatagaagaagaaggtggagaga
gagacagagacagatccattcgattagtgaacggatcggcactgcgtg
cgccaattctgcagacaaatggcagtattcatccacaattttaaaaga
aaaggggggattggggggtacagtgcaggggaaagaatagtagacata
atagcaacagacatacaaactaaagaattacaaaaacaaattacaaaa
attcaaaattttcgggtttattacagggacagcagagatccagtttgg
ttaattaatggggggacgttaacggggcggaacggtaccgagggccta
tttcccatgattccttcatatttgcatatacgatacaaggctgttaga
gagataattagaattaatttgactgtaaacacaaagatattagtacaa
aatacgtgacgtagaaagtaataatttcttgggtagtttgcagtttta
aaattatgttttaaaatggactatcatatgcttaccgtaacttgaaag
tatttcgatttcttggctttatatatcttgtggaaaggacgaaacacc
ggccctatccctgggggaggggttttagagctagaaatagcaagttaa
aataaggctagtccgttatcaacttgaaaaagtggcaccgagtcggtg
cttttttgaattcgctagctaggtcttgaaaggagtgggaattggctc
cggtgcccgtcagtgggcagagcgcacatcgcccacagtccccgagaa
gttggggggaggggtcggcaattgatccggtgcctagagaaggtggcg
cggggtaaactgggaaagtgatgtcgtgtactggctccgcctttttcc
cgaggggggggagaaccgtatataagtgcagtagtcgccgtgaacgtt
ctttttcgcaacgggtttgccgccagaacacaggaccggtgccaccat
ggactataaggaccacgacggagactacaaggatcatgatattgatta
caaagacgatgacgataagatggccccaaagaagaagcggaaggtcgg
tatccacggagtcccagcagccgacaagaagtacagcatcggcctggc
catcggcaccaactctgtgggctgggccgtgatcaccgacgagtacaa
ggtgcccagcaagaaattcaaggtgctgggcaacaccgaccggcacag
catcaagaagaacctgatcggagccctgctgttcgacagcggcgaaac
agccgaggccacccggctgaagagaaccgccagaagaagatacaccag
acggaagaaccggatctgctatctgcaagagatcttcagcaacgagat
ggccaaggtggacgacagcttcttccacagactggaagagtccttcct
ggtggaagaggataagaagcacgagcggcaccccatcttcggcaacat
cgtggacgaggtggcctaccacgagaagtaccccaccatctaccacct
gagaaagaaactggtggacagcaccgacaaggccgacctgcggctgat
ctatctggccctggcccacatgatcaagttccggggccacttcctgat
cgagggcgacctgaaccccgacaacagcgacgtggacaagctgttcat
ccagctggtgcagacctacaaccagctgttcgaggaaaaccccatcaa
cgccagcggcgtggacgccaaggccatcctgtctgccagactgagcaa
gagcagacggctggaaaatctgatcgcccagctgcccggcgagaagaa
gaatggcctgttcggcaacctgattgccctgagcctgggcctgacccc
caacttcaagagcaacttcgacctggccgaggatgccaaactgcagct
gagcaaggacacctacgacgacgacctggacaacctgctggcccagat
cggcgaccagtacgccgacctgtttctggccgccaagaacctgtccga
cgccatcctgctgagcgacatcctgagagtgaacaccgagatcaccaa
ggcccccctgagcgcctctatgatcaagagatacgacgagcaccacca
ggacctgaccctgctgaaagctctcgtgcggcagcagctgcctgagaa
gtacaaagagattttcttcgaccagagcaagaacggctacgccggcta
cattgacggcggagccagccaggaagagttctacaagttcatcaagcc
catcctggaaaagatggacggcaccgaggaactgctcgtgaagctgaa
cagagaggacctgctgcggaagcagcggaccttcgacaacggcagcat
cccccaccagatccacctgggagagctgcacgccattctgcggcggca
ggaagatttttacccattcctgaaggacaaccgggaaaagatcgagaa
gatcctgaccttccgcatcccctactacgtgggccctctggccagggg
aaacagcagattcgcctggatgaccagaaagagcgaggaaaccatcac
cccctggaacttcgaggaagtggtggacaagggcgcttccgcccagag
cttcatcgagcggatgaccaacttcgataagaacctgcccaacgagaa
ggtgctgcccaagcacagcctgctgtacgagtacttcaccgtgtataa
cgagctgaccaaagtgaaatacgtgaccgagggaatgagaaagcccgc
cttcctgagcggcgagcagaaaaaggccatcgtggacctgctgttcaa
gaccaaccggaaagtgaccgtgaagcagctgaaagaggactacttcaa
gaaaatcgagtgcttcgactccgtggaaatctccggcgtggaagatcg
gttcaacgcctccctgggcacataccacgatctgctgaaaattatcaa
ggacaaggacttcctggacaatgaggaaaacgaggacattctggaaga
tatcgtgctgaccctgacactgtttgaggacagagagatgatcgagga
acggctgaaaacctatgcccacctgttcgacgacaaagtgatgaagca
gctgaagcggcggagatacaccggctggggcaggctgagccggaagct
gatcaacggcatccgggacaagcagtccggcaagacaatcctggattt
cctgaagtccgacggcttcgccaacagaaacttcatgcagctgatcca
cgacgacagcctgacctttaaagaggacatccagaaagcccaggtgtc
cggccagggcgatagcctgcacgagcacattgccaatctggccggcag
ccccgccattaagaagggcatcctgcagacagtgaaggtggtggacga
gctcgtgaaagtgatgggccggcacaagcccgagaacatcgtgatcga
aatggccagagagaaccagaccacccagaagggacagaagaacagccg
cgagagaatgaagcggatcgaagagggcatcaaagagctgggcagcca
gatcctgaaagaacaccccgtggaaaacacccagctgcagaacgagaa
gctgtacctgtactacctgcagaatggggggatatgtacgtggaccag
gaactggacatcaaccggctgtccgactacgatgtggacgctatcgtg
cctcagagctttctgaaggacgactccatcgacaacaaggtgctgacc
agaagcgacaagaaccggggcaagagcgacaacgtgccctccgaagag
gtcgtgaagaagatgaagaactactggcggcagctgctgaacgccaag
ctgattacccagagaaagttcgacaatctgaccaaggccgagagaggc
ggcctgagcgaactggataaggccggcttcatcaagagacagctggtg
gaaacccggcagatcacaaagcacgtggcacagatactagattcccga
atgaatacgaaatacgacgagaacgataagctgattcgggaagtcaaa
gtaatcactttaaagtcaaaattggtgtcggacttcagaaaggatttt
caattctataaagttagggagataaataactaccaccatgcgcacgac
gcttatcttaatgccgtcgtagggaccgcactcattaagaaatacccg
aagctagaaagtgagtttgtgtatggtgattacaaagtttatgacgtc
cgtaagatgatcgcgaaaagcgaacaggagataggcaaggctacagcc
aaatacttcttttattctaacattatgaatttctttaagacggaaatc
actctggcaaacggagagatacgcaaacgacctttaattgaaaccaat
ggggagacaggtgaaatcgtatgggataagggccgggacttcgcgacg
gtgagaaaagttttgtccatgccccaagtcaacatagtaaagaaaact
gaggtgcagaccggagggttttcaaaggaatcgattcttccaaaaagg
aatagtgataagctcatcgctcgtaaaaaggactgggacccgaaaaag
tacggtggcttcgtgagccctacagttgcctattctgtcctagtagtg
gcaaaagttgagaagggaaaatccaagaaactgaagtcagtcaaagaa
ttattggggataacgattatggagcgctcgtcttttgaaaagaacccc
atcgacttccttgaggcgaaaggttacaaggaagtaaaaaaggatctc
ataattaaactaccaaagtatagtctgtttgagttagaaaatggccga
aaacggatgttggctagcgccagagagcttcaaaaggggaacgaactc
gcactaccgtctaaatacgtgaatttcctgtatttagcgtcccattac
gagaagttgaaaggttcacctgaagataacgaacagaagcaacttttt
gttgagcagcacaaacattatctcgacgaaatcatagagcaaatttcg
gaattcagtaagagagtcatcctagctgatgccaatctggacaaagta
ttaagcgcatacaacaagcacagggataaacccatacgtgagcaggcg
gaaaatattatccatttgtttactcttaccaacctcggcgctccagcc
gcattcaagtattttgacacaacgatagatcgcaaagagtacagatct
accaaggaggtgctagacgcgacactgattcaccaatccatcacggga
ttatatgaaactcggatagatttgtcacagcttgggggtgacggatcc
cccaagaagaagaggaaagtcctcgagggcggaggcgggagcggatcc
ccctcccggctccagatgttcttcgctaataaccacgaccaggaattt
gaccctccaaaggtttacccacctgtcccagctgagaagaggaagccc
atccgggtgctgtctctctttgatggaatcgctacagggctcctggtg
ctgaaggacttgggcattcaggtggaccgctacattgcctcggaggtg
tgtgaggactccatcacggtgggcatggtgcggcaccaggggaagatc
atgtacgtcggggacgtccgcagcgtcacacagaagcatatccaggag
tggggcccattcgatctggtgattgggggcagtccctgcaatgacctc
tccatcgtcaaccctgctcgcaagggcctctacgagggcactggccgg
ctcttctttgagttctaccgcctcctgcatgatgcgcggcccaaggag
ggagatgatcgccccttcttctggctctttgcgaatgtggtggccatg
ggcgttagtgacaagagggacatctcgcgatttctcgagtccaaccct
gtgatgattgatgccaaagaagtgtcagctgcacacagggcccgctac
ttctggggtaaccttcccggtatgaacaggccgttggcatccactgtg
aatgataagctggagctgcaggagtgtctggagcatggcaggatagcc
aagttcagcaaagtgaggaccattactacgaggtcaaactccataaag
cagggcaaagaccagcattttcctgtgttcatgaatgagaaagaggac
atcttatggtgcactgaaatggaaagggtatttggtttcccagtccac
tatactgacgtgtccaacatgagccgcttggcgaggcagagactgctg
ggccggtcatggagcgtgccagtcatccgccacctcttcgctccgctg
aaggagtattttgcgtgtgtgtccggccggcccggatccggcgcaaca
aacttctctctgctgaaacaagccggagatgtcgaagagaatcctgga
ccgaccgagtacaagcccacggtgcgcctcgccacccgcgacgacgtc
cccagggccgtacgcaccctcgccgccgcgttcgccgactaccccgcc
acgcgccacaccgtcgatccggaccgccacatcgagcgggtcaccgag
ctgcaagaactcttcctcacgcgcgtcgggctcgacatcggcaaggtg
tgggtcgcggacgacggcgccgcggtggcggtctggaccacgccggag
agcgtcgaagcgggggcggtgttcgccgagatcggcccgcgcatggcc
gagttgagcggttcccggctggccgcgcagcaacagatggaaggcctc
ctggcgccgcaccggcccaaggagcccgcgtggttcctggccaccgtc
ggagtctcgcccgaccaccagggcaagggtctgggcagcgccgtcgtg
ctccccggagtggaggcggccgagcgcgccggggtgcccgccttcctg
gagacctccgcgccccgcaacctccccttctacgagcggctcggcttc
accgtcaccgccgacgtcgaggtgcccgaaggaccgcgcacctggtgc
atgacccgcaagcccggtgcctgaacgcgttaagtcgacaatcaacct
ctggattacaaaatttgtgaaagattgactggtattcttaactatgtt
gctccttttacgctatgtggatacgctgctttaatgcctttgtatcat
gctattgcttcccgtatggctttcattttctcctccttgtataaatcc
tggttgctgtctctttatgaggagttgtggcccgttgtcaggcaacgt
ggcgtggtgtgcactgtgtttgctgacgcaacccccactggttggggc
attgccaccacctgtcagctcctttccgggactttcgctttccccctc
cctattgccacggcggaactcatcgccgcctgccttgcccgctgctgg
acaggggctcggctgttgggcactgacaattccgtggtgttgtcgggg
aaatcatcgtcctttccttggctgctcgcctgtgttgccacctggatt
ctgcgcgggacgtccttctgctacgtcccttcggccctcaatccagcg
gaccttccttcccgcggcctgctgccggctctgcggcctcttccgcgt
cttcgccttcgccctcagacgagtcggatctccctttgggccgcctcc
ccgcgtcgactttaagaccaatgacttacaaggcagctgtagatctta
gccactttttaaaagaaaaggggggactggaagggctaattcactccc
aacgaagacaagatctgctttttgcttgtactgggtctctctggttag
accagatctgagcctgggagctctctggctaactagggaacccactgc
ttaagcctcaataaagcttgccttgagtgcttcaagtagtgtgtgccc
gtctgttgtgtgactctggtaactagagatccctcagacccttttagt
cagtgtggaaaatctctagcagggcccgtttaaacccgctgatcagcc
tcgactgtgccttctagttgccagccatctgttgtttgcccctccccc
gtgccttccttgaccctggaaggtgccactcccactgtcctttcctaa
taaaatgaggaaattgcatcgcattgtctgagtaggtgtcattctatt
ctggggggtggggggggcaggacagcaagggggaggattgggaagaca
atagcaggcatgctggggatgcggtgggctctatggcttctgaggcgg
aaagaaccagctggggctctagggggtatccccacgcgccctgtagcg
gcgcattaagcgcggcgggtgtggtggttacgcgcagcgtgaccgcta
cacttgccagcgccctagcgcccgctcctttcgctttcttcccttcct
ttctcgccacgttcgccggctttccccgtcaagctctaaatcgggggc
tccctttagggttccgatttagtgctttacggcacctcgaccccaaaa
aacttgattagggtgatggttcacgtagtgggccatcgccctgataga
cggtttttcgccctttgacgttggagtccacgttctttaatagtggac
tcttgttccaaactggaacaacactcaaccctatctcggtctattctt
ttgatttataagggattttgccgatttcggcctattggttaaaaaatg
agctgatttaacaaaaatttaacgcgaattaattctgtggaatgtgtg
tcagttagggtgtggaaagtccccaggctccccagcaggcagaagtat
gcaaagcatgcatctcaattagtcagcaaccaggtgtggaaagtcccc
aggctccccagcaggcagaagtatgcaaagcatgcatctcaattagtc
agcaaccatagtcccgcccctaactccgcccatcccgcccctaactcc
gcccagttccgcccattctccgccccatggctgactaattttttttat
ttatgcagaggccgaggccgcctctgcctctgagctattccagaagta
gtgaggaggcttttttggaggcctaggcttttgcaaaaagctccctac
cgtcgacctctagctagagcttggcgtaatcatggtcatagctgtttc
ctgtgtgaaattgttatccgctcacaattccacacaacatacgagccg
gaagcataaagtgtaaagcctggggtgcctaatgagtgagctaactca
cattaattgcgttgcgctcactgcccgctttccagtcgggaaacctgt
cgtgccagctgcattaatgaatcggccaacgcgcggggagaggcggtt
tgcgtattgggcgctcttccgcttcctcgctcactgactcgctgcgct
cggtcgttcggctgcggcgagcggtatcagctcactcaaaggcggtaa
tacggttatccacagaatcaggggataacgcaggaaagaacatgtgag
caaaaggccagcaaaaggccaggaaccgtaaaaaggccgcgttgctgg
cgtttttccataggctccgcccccctgacgagcatcacaaaaatcgac
gctcaagtcagaggtggcgaaacccgacaggactataaagataccagg
cgtttccccctggaagctccctcgtgcgctctcctgttccgaccctgc
cgcttaccggatacctgtccgcctttctcccttcgggaagcgtggcgc
tttctcatagctcacgctgtaggtatctcagttcggtgtaggtcgttc
gctccaagctgggctgtgtgcacgaaccccccgttcagcccgaccgct
gcgccttatccggtaactatcgtcttgagtccaacccggtaagacacg
acttatcgccactggcagcagccactggtaacaggattagcagagcga
ggtatgtaggcggtgctacagagttcttgaagtggtggcctaactacg
gctacactagaagaacagtatttggtatctgcgctctgctgaagccag
ttaccttcggaaaaagagttggtagctcttgatccggcaaacaaacca
ccgctggtagcggtggtttttttgtttgcaagcagcagattacgcgca
gaaaaaaaggatctcaagaagatcctttgatcttttctacggggtctg
acgctcagtggaacgaaaactcacgttaagggattttggtcatgagat
tatcaaaaaggatcttcacctagatccttttaaattaaaaatgaagtt
ttaaatcaatctaaagtatatatgagtaaacttggtctgacagttacc
aatgcttaatcagtgaggcacctatctcagcgatctgtctatttcgtt
catccatagttgcctgactccccgtcgtgtagataactacgatacggg
agggcttaccatctggccccagtgctgcaatgataccgcgagacccac
gctcaccggctccagatttatcagcaataaaccagccagccggaaggg
ccgagcgcagaagtggtcctgcaactttatccgcctccatccagtcta
ttaattgttgccgggaagctagagtaagtagttcgccagttaatagtt
tgcgcaacgttgttgccattgctacaggcatcgtggtgtcacgctcgt
cgtttggtatggcttcattcagctccggttcccaacgatcaaggcgag
ttacatgatcccccatgttgtgcaaaaaagcggttagctccttcggtc
ctccgatcgttgtcagaagtaagttggccgcagtgttatcactcatgg
ttatggcagcactgcataattctcttactgtcatgccatccgtaagat
gcttttctgtgactggtgagtactcaaccaagtcattctgagaatagt
gtatgcggcgaccgagttgctcttgcccggcgtcaatacgggataata
ccgcgccacatagcagaactttaaaagtgctcatcattggaaaacgtt
cttcggggcgaaaactctcaaggatcttaccgctgttgagatccagtt
cgatgtaacccactcgtgcacccaactgatcttcagcatcttttactt
tcaccagcgtttctgggtgagcaaaaacaggaaggcaaaatgccgcaa
aaaagggaataagggcgacacggaaatgttgaatactcatactcttcc
tttttcaatattattgaagcatttatcagggttattgtctcatgagcg
gatacatatttgaatgtatttagaaaaataaacaaataggggttccgc
gcacatttccccgaaaagtgccacctgac.

In an aspect, a disclosed pBK1110 plasmid can comprise the sequence set forth below or a sequence having 70%, 75%, 80%, 85%, 90%, 95%, or more identity to the sequence set forth

(SEQ ID NO: 48)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggtggagtatttacggtaaactgcccacttggcagtacatcaag
tgtatcatatgccaagtacgccccctattgacgtcaatgacggtaaat
ggcccgcctggcattatgcccagtacatgaccttatgggactttccta
cttggcagtacatctacgtattagtcatcgctattaccatggtgatgc
ggttttggcagtacatcaatgggcgtggatagcggtttgactcacggg
gatttccaagtctccaccccattgacgtcaatgggagtttgttttggc
accaaaatcaacgggactttccaaaatgtcgtaacaactccgccccat
tgacgcaaatgggcggtaggcgtgtacggtgggaggtctatataagca
gcgcgttttgcctgtactgggtctctctggttagaccagatctgagcc
tgggagctctctggctaactagggaacccactgcttaagcctcaataa
agcttgccttgagtgcttcaagtagtgtgtgcccgtctgttgtgtgac
tctggtaactagagatccctcagacccttttagtcagtgtggaaaatc
tctagcagtggcgcccgaacagggacttgaaagcgaaagggaaaccag
aggagctctctcgacgcaggactcggcttgctgaagcgcgcacggcaa
gaggcgaggggcggcgactggtgagtacgccaaaaattttgactagcg
gaggctagaaggagagagatgggtgcgagagcgtcagtattaagcggg
ggagaattagatcgcgatgggaaaaaattcggttaaggccagggggaa
agaaaaaatataaattaaaacatatagtatgggcaagcagggagctag
aacgattcgcagttaatcctggcctgttagaaacatcagaaggctgta
gacaaatactgggacagctacaaccatcccttcagacaggatcagaag
aacttagatcattatataatacagtagcaaccctctattgtgtgcatc
aaaggatagagataaaagacaccaaggaagctttagacaagatagagg
aagagcaaaacaaaagtaagaccaccgcacagcaagcggccgctgatc
ttcagacctggaggaggagatatgagggacaattggagaagtgaatta
tataaatataaagtagtaaaaattgaaccattaggagtagcacccacc
aaggcaaagagaagagtggtgcagagagaaaaaagagcagtgggaata
ggagctttgttccttgggttcttgggagcagcaggaagcactatgggc
gcagcgtcaatgacgctgacggtacaggccagacaattattgtctggt
atagtgcagcagcagaacaatttgctgagggctattgaggcgcaacag
catctgttgcaactcacagtctggggcatcaagcagctccaggcaaga
atcctggctgtggaaagatacctaaaggatcaacagctcctggggatt
tggggttgctctggaaaactcatttgcaccactgctgtgccttggaat
gctagttggagtaataaatctctggaacagatttggaatcacacgacc
tggatggagtgggacagagaaattaacaattacacaagcttaatacac
tccttaattgaagaatcgcaaaaccagcaagaaaagaatgaacaagaa
ttattggaattagataaatgggcaagtttgtggaattggtttaacata
acaaattggctgtggtatataaaattattcataatgatagtaggaggc
ttggtaggtttaagaatagtttttgctgtactttctatagtgaataga
gttaggcagggatattcaccattatcgtttcagacccacctcccaacc
ccgaggggacccgacaggcccgaaggaatagaagaagaaggtggagag
agagacagagacagatccattcgattagtgaacggatcggcactgcgt
gcgccaattctgcagacaaatggcagtattcatccacaattttaaaag
aaaaggggggattggggggtacagtgcaggggaaagaatagtagacat
aatagcaacagacatacaaactaaagaattacaaaaacaaattacaaa
aattcaaaattttcgggtttattacagggacagcagagatccagtttg
gttaattaatggggggacgttaacggggcggaacggtaccgagggcct
atttcccatgattccttcatatttgcatatacgatacaaggctgttag
agagataattagaattaatttgactgtaaacacaaagatattagtaca
aaatacgtgacgtagaaagtaataatttcttgggtagtttgcagtttt
aaaattatgttttaaaatggactatcatatgcttaccgtaacttgaaa
gtatttcgatttcttggctttatatatcttgtggaaaggacgaaacac
cgtcgggcttggggagaggagggttttagagctagaaatagcaagtta
aaataaggctagtccgttatcaacttgaaaaagtggcaccgagtcggt
gcttttttgaattcgctagctaggtcttgaaaggagtgggaattggct
ccggtgcccgtcagtgggcagagcgcacatcgcccacagtccccgaga
agttggggggaggggtcggcaattgatccggtgcctagagaaggtggc
gcggggtaaactgggaaagtgatgtcgtgtactggctccgcctttttc
ccgaggggggggagaaccgtatataagtgcagtagtcgccgtgaacgt
tctttttcgcaacgggtttgccgccagaacacaggaccggtgccacca
tggactataaggaccacgacggagactacaaggatcatgatattgatt
acaaagacgatgacgataagatggccccaaagaagaagcggaaggtcg
gtatccacggagtcccagcagccgacaagaagtacagcatcggcctgg
ccatcggcaccaactctgtgggctgggccgtgatcaccgacgagtaca
aggtgcccagcaagaaattcaaggtgctgggcaacaccgaccggcaca
gcatcaagaagaacctgatcggagccctgctgttcgacagcggcgaaa
cagccgaggccacccggctgaagagaaccgccagaagaagatacacca
gacggaagaaccggatctgctatctgcaagagatcttcagcaacgaga
tggccaaggtggacgacagcttcttccacagactggaagagtccttcc
tggtggaagaggataagaagcacgagcggcaccccatcttcggcaaca
tcgtggacgaggtggcctaccacgagaagtaccccaccatctaccacc
tgagaaagaaactggtggacagcaccgacaaggccgacctgcggctga
tctatctggccctggcccacatgatcaagttccggggccacttcctga
tcgagggcgacctgaaccccgacaacagcgacgtggacaagctgttca
tccagctggtgcagacctacaaccagctgttcgaggaaaaccccatca
acgccagcggcgtggacgccaaggccatcctgtctgccagactgagca
agagcagacggctggaaaatctgatcgcccagctgcccggcgagaaga
agaatggcctgttcggcaacctgattgccctgagcctgggcctgaccc
ccaacttcaagagcaacttcgacctggccgaggatgccaaactgcagc
tgagcaaggacacctacgacgacgacctggacaacctgctggcccaga
tcggcgaccagtacgccgacctgtttctggccgccaagaacctgtccg
acgccatcctgctgagcgacatcctgagagtgaacaccgagatcacca
aggcccccctgagcgcctctatgatcaagagatacgacgagcaccacc
aggacctgaccctgctgaaagctctcgtgcggcagcagctgcctgaga
agtacaaagagattttcttcgaccagagcaagaacggctacgccggct
acattgacggcggagccagccaggaagagttctacaagttcatcaagc
ccatcctggaaaagatggacggcaccgaggaactgctcgtgaagctga
acagagaggacctgctgcggaagcagcggaccttcgacaacggcagca
tcccccaccagatccacctgggagagctgcacgccattctgcggcggc
aggaagatttttacccattcctgaaggacaaccgggaaaagatcgaga
agatcctgaccttccgcatcccctactacgtgggccctctggccaggg
gaaacagcagattcgcctggatgaccagaaagagcgaggaaaccatca
ccccctggaacttcgaggaagtggtggacaagggcgcttccgcccaga
gcttcatcgagcggatgaccaacttcgataagaacctgcccaacgaga
aggtgctgcccaagcacagcctgctgtacgagtacttcaccgtgtata
acgagctgaccaaagtgaaatacgtgaccgagggaatgagaaagcccg
ccttcctgagcggcgagcagaaaaaggccatcgtggacctgctgttca
agaccaaccggaaagtgaccgtgaagcagctgaaagaggactacttca
agaaaatcgagtgcttcgactccgtggaaatctccggcgtggaagatc
ggttcaacgcctccctgggcacataccacgatctgctgaaaattatca
aggacaaggacttcctggacaatgaggaaaacgaggacattctggaag
atatcgtgctgaccctgacactgtttgaggacagagagatgatcgagg
aacggctgaaaacctatgcccacctgttcgacgacaaagtgatgaagc
agctgaagcggcggagatacaccggctggggcaggctgagccggaagc
tgatcaacggcatccgggacaagcagtccggcaagacaatcctggatt
tcctgaagtccgacggcttcgccaacagaaacttcatgcagctgatcc
acgacgacagcctgacctttaaagaggacatccagaaagcccaggtgt
ccggccagggcgatagcctgcacgagcacattgccaatctggccggca
gccccgccattaagaagggcatcctgcagacagtgaaggtggtggacg
agctcgtgaaagtgatgggccggcacaagcccgagaacatcgtgatcg
aaatggccagagagaaccagaccacccagaagggacagaagaacagcc
gcgagagaatgaagcggatcgaagagggcatcaaagagctgggcagcc
agatcctgaaagaacaccccgtggaaaacacccagctgcagaacgaga
agctgtacctgtactacctgcagaatggggggatatgtacgtggacca
ggaactggacatcaaccggctgtccgactacgatgtggacgctatcgt
gcctcagagctttctgaaggacgactccatcgacaacaaggtgctgac
cagaagcgacaagaaccggggcaagagcgacaacgtgccctccgaaga
ggtcgtgaagaagatgaagaactactggcggcagctgctgaacgccaa
gctgattacccagagaaagttcgacaatctgaccaaggccgagagagg
cggcctgagcgaactggataaggccggcttcatcaagagacagctggt
ggaaacccggcagatcacaaagcacgtggcacagatactagattcccg
aatgaatacgaaatacgacgagaacgataagctgattcgggaagtcaa
agtaatcactttaaagtcaaaattggtgtcggacttcagaaaggattt
tcaattctataaagttagggagataaataactaccaccatgcgcacga
cgcttatcttaatgccgtcgtagggaccgcactcattaagaaataccc
gaagctagaaagtgagtttgtgtatggtgattacaaagtttatgacgt
ccgtaagatgatcgcgaaaagcgaacaggagataggcaaggctacagc
caaatacttcttttattctaacattatgaatttctttaagacggaaat
cactctggcaaacggagagatacgcaaacgacctttaattgaaaccaa
tggggagacaggtgaaatcgtatgggataagggccgggacttcgcgac
ggtgagaaaagttttgtccatgccccaagtcaacatagtaaagaaaac
tgaggtgcagaccggagggttttcaaaggaatcgattcttccaaaaag
gaatagtgataagctcatcgctcgtaaaaaggactgggacccgaaaaa
gtacggtggcttcgtgagccctacagttgcctattctgtcctagtagt
ggcaaaagttgagaagggaaaatccaagaaactgaagtcagtcaaaga
attattggggataacgattatggagcgctcgtcttttgaaaagaaccc
catcgacttccttgaggcgaaaggttacaaggaagtaaaaaaggatct
cataattaaactaccaaagtatagtctgtttgagttagaaaatggccg
aaaacggatgttggctagcgccagagagcttcaaaaggggaacgaact
cgcactaccgtctaaatacgtgaatttcctgtatttagcgtcccatta
cgagaagttgaaaggttcacctgaagataacgaacagaagcaactttt
tgttgagcagcacaaacattatctcgacgaaatcatagagcaaatttc
ggaattcagtaagagagtcatcctagctgatgccaatctggacaaagt
attaagcgcatacaacaagcacagggataaacccatacgtgagcaggc
ggaaaatattatccatttgtttactcttaccaacctcggcgctccagc
cgcattcaagtattttgacacaacgatagatcgcaaagagtacagatc
taccaaggaggtgctagacgcgacactgattcaccaatccatcacggg
attatatgaaactcggatagatttgtcacagcttgggggtgacggatc
ccccaagaagaagaggaaagtcctcgagggggaggcgggagcggatcc
ccctcccggctccagatgttcttcgctaataaccacgaccaggaattt
gaccctccaaaggtttacccacctgtcccagctgagaagaggaagccc
atccgggtgctgtctctctttgatggaatcgctacagggctcctggtg
ctgaaggacttgggcattcaggtggaccgctacattgcctcggaggtg
tgtgaggactccatcacggtgggcatggtgcggcaccaggggaagatc
atgtacgtcggggacgtccgcagcgtcacacagaagcatatccaggag
tggggcccattcgatctggtgattgggggcagtccctgcaatgacctc
tccatcgtcaaccctgctcgcaagggcctctacgagggcactggccgg
ctcttctttgagttctaccgcctcctgcatgatgcgcggcccaaggag
ggagatgatcgccccttcttctggctctttgcgaatgtggtggccatg
ggcgttagtgacaagagggacatctcgcgatttctcgagtccaaccct
gtgatgattgatgccaaagaagtgtcagctgcacacagggcccgctac
ttctggggtaaccttcccggtatgaacaggccgttggcatccactgtg
aatgataagctggagctgcaggagtgtctggagcatggcaggatagcc
aagttcagcaaagtgaggaccattactacgaggtcaaactccataaag
cagggcaaagaccagcattttcctgtgttcatgaatgagaaagaggac
atcttatggtgcactgaaatggaaagggtatttggtttcccagtccac
tatactgacgtgtccaacatgagccgcttggcgaggcagagactgctg
ggccggtcatggagcgtgccagtcatccgccacctcttcgctccgctg
aaggagtattttgcgtgtgtgtccggccggcccggatccggcgcaaca
aacttctctctgctgaaacaagccggagatgtcgaagagaatcctgga
ccgaccgagtacaagcccacggtgcgcctcgccacccgcgacgacgtc
cccagggccgtacgcaccctcgccgccgcgttcgccgactaccccgcc
acgcgccacaccgtcgatccggaccgccacatcgagcgggtcaccgag
ctgcaagaactcttcctcacgcgcgtcgggctcgacatcggcaaggtg
tgggtcgcggacgacggcgccgcggtggcggtctggaccacgccggag
agcgtcgaagcgggggcggtgttcgccgagatcggcccgcgcatggcc
gagttgagcggttcccggctggccgcgcagcaacagatggaaggcctc
ctggcgccgcaccggcccaaggagcccgcgtggttcctggccaccgtc
ggagtctcgcccgaccaccagggcaagggtctgggcagcgccgtcgtg
ctccccggagtggaggcggccgagcgcgccggggtgcccgccttcctg
gagacctccgcgccccgcaacctccccttctacgagcggctcggcttc
accgtcaccgccgacgtcgaggtgcccgaaggaccgcgcacctggtgc
atgacccgcaagcccggtgcctgaacgcgttaagtcgacaatcaacct
ctggattacaaaatttgtgaaagattgactggtattcttaactatgtt
gctccttttacgctatgtggatacgctgctttaatgcctttgtatcat
gctattgcttcccgtatggctttcattttctcctccttgtataaatcc
tggttgctgtctctttatgaggagttgtggcccgttgtcaggcaacgt
ggcgtggtgtgcactgtgtttgctgacgcaacccccactggttggggc
attgccaccacctgtcagctcctttccgggactttcgctttccccctc
cctattgccacggcggaactcatcgccgcctgccttgcccgctgctgg
acaggggctcggctgttgggcactgacaattccgtggtgttgtcgggg
aaatcatcgtcctttccttggctgctcgcctgtgttgccacctggatt
ctgcgcgggacgtccttctgctacgtcccttcggccctcaatccagcg
gaccttccttcccgcggcctgctgccggctctgcggcctcttccgcgt
cttcgccttcgccctcagacgagtcggatctccctttgggccgcctcc
ccgcgtcgactttaagaccaatgacttacaaggcagctgtagatctta
gccactttttaaaagaaaaggggggactggaagggctaattcactccc
aacgaagacaagatctgctttttgcttgtactgggtctctctggttag
accagatctgagcctgggagctctctggctaactagggaacccactgc
ttaagcctcaataaagcttgccttgagtgcttcaagtagtgtgtgccc
gtctgttgtgtgactctggtaactagagatccctcagacccttttagt
cagtgtggaaaatctctagcagggcccgtttaaacccgctgatcagcc
tcgactgtgccttctagttgccagccatctgttgtttgcccctccccc
gtgccttccttgaccctggaaggtgccactcccactgtcctttcctaa
taaaatgaggaaattgcatcgcattgtctgagtaggtgtcattctatt
ctggggggtggggggggcaggacagcaagggggaggattgggaagaca
atagcaggcatgctggggatgcggtgggctctatggcttctgaggcgg
aaagaaccagctggggctctagggggtatccccacgcgccctgtagcg
gcgcattaagcgcggcgggtgtggtggttacgcgcagcgtgaccgcta
cacttgccagcgccctagcgcccgctcctttcgctttcttcccttcct
ttctcgccacgttcgccggctttccccgtcaagctctaaatcgggggc
tccctttagggttccgatttagtgctttacggcacctcgaccccaaaa
aacttgattagggtgatggttcacgtagtgggccatcgccctgataga
cggtttttcgccctttgacgttggagtccacgttctttaatagtggac
tcttgttccaaactggaacaacactcaaccctatctcggtctattctt
ttgatttataagggattttgccgatttcggcctattggttaaaaaatg
agctgatttaacaaaaatttaacgcgaattaattctgtggaatgtgtg
tcagttagggtgtggaaagtccccaggctccccagcaggcagaagtat
gcaaagcatgcatctcaattagtcagcaaccaggtgtggaaagtcccc
aggctccccagcaggcagaagtatgcaaagcatgcatctcaattagtc
agcaaccatagtcccgcccctaactccgcccatcccgcccctaactcc
gcccagttccgcccattctccgccccatggctgactaattttttttat
ttatgcagaggccgaggccgcctctgcctctgagctattccagaagta
gtgaggaggcttttttggaggcctaggcttttgcaaaaagctccctac
cgtcgacctctagctagagcttggcgtaatcatggtcatagctgtttc
ctgtgtgaaattgttatccgctcacaattccacacaacatacgagccg
gaagcataaagtgtaaagcctggggtgcctaatgagtgagctaactca
cattaattgcgttgcgctcactgcccgctttccagtcgggaaacctgt
cgtgccagctgcattaatgaatcggccaacgcgcggggagaggcggtt
tgcgtattgggcgctcttccgcttcctcgctcactgactcgctgcgct
cggtcgttcggctgcggcgagcggtatcagctcactcaaaggcggtaa
tacggttatccacagaatcaggggataacgcaggaaagaacatgtgag
caaaaggccagcaaaaggccaggaaccgtaaaaaggccgcgttgctgg
cgtttttccataggctccgcccccctgacgagcatcacaaaaatcgac
gctcaagtcagaggtggcgaaacccgacaggactataaagataccagg
cgtttccccctggaagctccctcgtgcgctctcctgttccgaccctgc
cgcttaccggatacctgtccgcctttctcccttcgggaagcgtggcgc
tttctcatagctcacgctgtaggtatctcagttcggtgtaggtcgttc
gctccaagctgggctgtgtgcacgaaccccccgttcagcccgaccgct
gcgccttatccggtaactatcgtcttgagtccaacccggtaagacacg
acttatcgccactggcagcagccactggtaacaggattagcagagcga
ggtatgtaggcggtgctacagagttcttgaagtggtggcctaactacg
gctacactagaagaacagtatttggtatctgcgctctgctgaagccag
ttaccttcggaaaaagagttggtagctcttgatccggcaaacaaacca
ccgctggtagcggtggtttttttgtttgcaagcagcagattacgcgca
gaaaaaaaggatctcaagaagatcctttgatcttttctacggggtctg
acgctcagtggaacgaaaactcacgttaagggattttggtcatgagat
tatcaaaaaggatcttcacctagatccttttaaattaaaaatgaagtt
ttaaatcaatctaaagtatatatgagtaaacttggtctgacagttacc
aatgcttaatcagtgaggcacctatctcagcgatctgtctatttcgtt
catccatagttgcctgactccccgtcgtgtagataactacgatacggg
agggcttaccatctggccccagtgctgcaatgataccgcgagacccac
gctcaccggctccagatttatcagcaataaaccagccagccggaaggg
ccgagcgcagaagtggtcctgcaactttatccgcctccatccagtcta
ttaattgttgccgggaagctagagtaagtagttcgccagttaatagtt
tgcgcaacgttgttgccattgctacaggcatcgtggtgtcacgctcgt
cgtttggtatggcttcattcagctccggttcccaacgatcaaggcgag
ttacatgatcccccatgttgtgcaaaaaagcggttagctccttcggtc
ctccgatcgttgtcagaagtaagttggccgcagtgttatcactcatgg
ttatggcagcactgcataattctcttactgtcatgccatccgtaagat
gcttttctgtgactggtgagtactcaaccaagtcattctgagaatagt
gtatgcggcgaccgagttgctcttgcccggcgtcaatacgggataata
ccgcgccacatagcagaactttaaaagtgctcatcattggaaaacgtt
cttcggggcgaaaactctcaaggatcttaccgctgttgagatccagtt
cgatgtaacccactcgtgcacccaactgatcttcagcatcttttactt
tcaccagcgtttctgggtgagcaaaaacaggaaggcaaaatgccgcaa
aaaagggaataagggcgacacggaaatgttgaatactcatactcttcc
tttttcaatattattgaagcatttatcagggttattgtctcatgagcg
gatacatatttgaatgtatttagaaaaataaacaaataggggttccgc
gcacatttccccgaaaagtgccacctgac.

In an aspect, a disclosed pBK1111 plasmid can comprise the sequence set forth below or a sequence having 70%, 75%, 80%, 85%, 90%, 95%, or more identity to the sequence set forth

(SEQ ID NO: 49)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggtggagtatttacggtaaactgcccacttggcagtacatcaag
tgtatcatatgccaagtacgccccctattgacgtcaatgacggtaaat
ggcccgcctggcattatgcccagtacatgaccttatgggactttccta
cttggcagtacatctacgtattagtcatcgctattaccatggtgatgc
ggttttggcagtacatcaatgggcgtggatagcggtttgactcacggg
gatttccaagtctccaccccattgacgtcaatgggagtttgttttggc
accaaaatcaacgggactttccaaaatgtcgtaacaactccgccccat
tgacgcaaatgggcggtaggcgtgtacggtgggaggtctatataagca
gcgcgttttgcctgtactgggtctctctggttagaccagatctgagcc
tgggagctctctggctaactagggaacccactgcttaagcctcaataa
agcttgccttgagtgcttcaagtagtgtgtgcccgtctgttgtgtgac
tctggtaactagagatccctcagacccttttagtcagtgtggaaaatc
tctagcagtggcgcccgaacagggacttgaaagcgaaagggaaaccag
aggagctctctcgacgcaggactcggcttgctgaagcgcgcacggcaa
gaggcgaggggcggcgactggtgagtacgccaaaaattttgactagcg
gaggctagaaggagagagatgggtgcgagagcgtcagtattaagcggg
ggagaattagatcgcgatgggaaaaaattcggttaaggccagggggaa
agaaaaaatataaattaaaacatatagtatgggcaagcagggagctag
aacgattcgcagttaatcctggcctgttagaaacatcagaaggctgta
gacaaatactgggacagctacaaccatcccttcagacaggatcagaag
aacttagatcattatataatacagtagcaaccctctattgtgtgcatc
aaaggatagagataaaagacaccaaggaagctttagacaagatagagg
aagagcaaaacaaaagtaagaccaccgcacagcaagcggccgctgatc
ttcagacctggaggaggagatatgagggacaattggagaagtgaatta
tataaatataaagtagtaaaaattgaaccattaggagtagcacccacc
aaggcaaagagaagagtggtgcagagagaaaaaagagcagtgggaata
ggagctttgttccttgggttcttgggagcagcaggaagcactatgggc
gcagcgtcaatgacgctgacggtacaggccagacaattattgtctggt
atagtgcagcagcagaacaatttgctgagggctattgaggcgcaacag
catctgttgcaactcacagtctggggcatcaagcagctccaggcaaga
atcctggctgtggaaagatacctaaaggatcaacagctcctggggatt
tggggttgctctggaaaactcatttgcaccactgctgtgccttggaat
gctagttggagtaataaatctctggaacagatttggaatcacacgacc
tggatggagtgggacagagaaattaacaattacacaagcttaatacac
tccttaattgaagaatcgcaaaaccagcaagaaaagaatgaacaagaa
ttattggaattagataaatgggcaagtttgtggaattggtttaacata
acaaattggctgtggtatataaaattattcataatgatagtaggaggc
ttggtaggtttaagaatagtttttgctgtactttctatagtgaataga
gttaggcagggatattcaccattatcgtttcagacccacctcccaacc
ccgaggggacccgacaggcccgaaggaatagaagaagaaggtggagag
agagacagagacagatccattcgattagtgaacggatcggcactgcgt
gcgccaattctgcagacaaatggcagtattcatccacaattttaaaag
aaaaggggggattggggggtacagtgcaggggaaagaatagtagacat
aatagcaacagacatacaaactaaagaattacaaaaacaaattacaaa
aattcaaaattttcgggtttattacagggacagcagagatccagtttg
gttaattaatggggggacgttaacggggggaacggtaccgagggccta
tttcccatgattccttcatatttgcatatacgatacaaggctgttaga
gagataattagaattaatttgactgtaaacacaaagatattagtacaa
aatacgtgacgtagaaagtaataatttcttgggtagtttgcagtttta
aaattatgttttaaaatggactatcatatgcttaccgtaacttgaaag
tatttcgatttcttggctttatatatcttgtggaaaggacgaaacacc
gctctccccaccccaccttctgttttagagctagaaatagcaagttaa
aataaggctagtccgttatcaacttgaaaaagtggcaccgagtcggtg
cttttttgaattcgctagctaggtcttgaaaggagtgggaattggctc
cggtgcccgtcagtgggcagagcgcacatcgcccacagtccccgagaa
gttggggggaggggtcggcaattgatccggtgcctagagaaggtggcg
cggggtaaactgggaaagtgatgtcgtgtactggctccgcctttttcc
cgaggggggggagaaccgtatataagtgcagtagtcgccgtgaacgtt
ctttttcgcaacgggtttgccgccagaacacaggaccggtgccaccat
ggactataaggaccacgacggagactacaaggatcatgatattgatta
caaagacgatgacgataagatggccccaaagaagaagcggaaggtcgg
tatccacggagtcccagcagccgacaagaagtacagcatcggcctggc
catcggcaccaactctgtgggctgggccgtgatcaccgacgagtacaa
ggtgcccagcaagaaattcaaggtgctgggcaacaccgaccggcacag
catcaagaagaacctgatcggagccctgctgttcgacagcggcgaaac
agccgaggccacccggctgaagagaaccgccagaagaagatacaccag
acggaagaaccggatctgctatctgcaagagatcttcagcaacgagat
ggccaaggtggacgacagcttcttccacagactggaagagtccttcct
ggtggaagaggataagaagcacgagcggcaccccatcttcggcaacat
cgtggacgaggtggcctaccacgagaagtaccccaccatctaccacct
gagaaagaaactggtggacagcaccgacaaggccgacctgcggctgat
ctatctggccctggcccacatgatcaagttccggggccacttcctgat
cgagggcgacctgaaccccgacaacagcgacgtggacaagctgttcat
ccagctggtgcagacctacaaccagctgttcgaggaaaaccccatcaa
cgccagcggcgtggacgccaaggccatcctgtctgccagactgagcaa
gagcagacggctggaaaatctgatcgcccagctgcccggcgagaagaa
gaatggcctgttcggcaacctgattgccctgagcctgggcctgacccc
caacttcaagagcaacttcgacctggccgaggatgccaaactgcagct
gagcaaggacacctacgacgacgacctggacaacctgctggcccagat
cggcgaccagtacgccgacctgtttctggccgccaagaacctgtccga
cgccatcctgctgagcgacatcctgagagtgaacaccgagatcaccaa
ggcccccctgagcgcctctatgatcaagagatacgacgagcaccacca
ggacctgaccctgctgaaagctctcgtgcggcagcagctgcctgagaa
gtacaaagagattttcttcgaccagagcaagaacggctacgccggcta
cattgacggcggagccagccaggaagagttctacaagttcatcaagcc
catcctggaaaagatggacggcaccgaggaactgctcgtgaagctgaa
cagagaggacctgctgcggaagcagcggaccttcgacaacggcagcat
cccccaccagatccacctgggagagctgcacgccattctgcggcggca
ggaagatttttacccattcctgaaggacaaccgggaaaagatcgagaa
gatcctgaccttccgcatcccctactacgtgggccctctggccagggg
aaacagcagattcgcctggatgaccagaaagagcgaggaaaccatcac
cccctggaacttcgaggaagtggtggacaagggcgcttccgcccagag
cttcatcgagcggatgaccaacttcgataagaacctgcccaacgagaa
ggtgctgcccaagcacagcctgctgtacgagtacttcaccgtgtataa
cgagctgaccaaagtgaaatacgtgaccgagggaatgagaaagcccgc
cttcctgagcggcgagcagaaaaaggccatcgtggacctgctgttcaa
gaccaaccggaaagtgaccgtgaagcagctgaaagaggactacttcaa
gaaaatcgagtgcttcgactccgtggaaatctccggcgtggaagatcg
gttcaacgcctccctgggcacataccacgatctgctgaaaattatcaa
ggacaaggacttcctggacaatgaggaaaacgaggacattctggaaga
tatcgtgctgaccctgacactgtttgaggacagagagatgatcgagga
acggctgaaaacctatgcccacctgttcgacgacaaagtgatgaagca
gctgaagcggcggagatacaccggctggggcaggctgagccggaagct
gatcaacggcatccgggacaagcagtccggcaagacaatcctggattt
cctgaagtccgacggcttcgccaacagaaacttcatgcagctgatcca
cgacgacagcctgacctttaaagaggacatccagaaagcccaggtgtc
cggccagggcgatagcctgcacgagcacattgccaatctggccggcag
ccccgccattaagaagggcatcctgcagacagtgaaggtggtggacga
gctcgtgaaagtgatgggccggcacaagcccgagaacatcgtgatcga
aatggccagagagaaccagaccacccagaagggacagaagaacagccg
cgagagaatgaagcggatcgaagagggcatcaaagagctgggcagcca
gatcctgaaagaacaccccgtggaaaacacccagctgcagaacgagaa
gctgtacctgtactacctgcagaatgggcgggatatgtacgtggacca
ggaactggacatcaaccggctgtccgactacgatgtggacgctatcgt
gcctcagagctttctgaaggacgactccatcgacaacaaggtgctgac
cagaagcgacaagaaccggggcaagagcgacaacgtgccctccgaaga
ggtcgtgaagaagatgaagaactactggcggcagctgctgaacgccaa
gctgattacccagagaaagttcgacaatctgaccaaggccgagagagg
cggcctgagcgaactggataaggccggcttcatcaagagacagctggt
ggaaacccggcagatcacaaagcacgtggcacagatactagattcccg
aatgaatacgaaatacgacgagaacgataagctgattcgggaagtcaa
agtaatcactttaaagtcaaaattggtgtcggacttcagaaaggattt
tcaattctataaagttagggagataaataactaccaccatgcgcacga
cgcttatcttaatgccgtcgtagggaccgcactcattaagaaataccc
gaagctagaaagtgagtttgtgtatggtgattacaaagtttatgacgt
ccgtaagatgatcgcgaaaagcgaacaggagataggcaaggctacagc
caaatacttcttttattctaacattatgaatttctttaagacggaaat
cactctggcaaacggagagatacgcaaacgacctttaattgaaaccaa
tggggagacaggtgaaatcgtatgggataagggccgggacttcgcgac
ggtgagaaaagttttgtccatgccccaagtcaacatagtaaagaaaac
tgaggtgcagaccggagggttttcaaaggaatcgattcttccaaaaag
gaatagtgataagctcatcgctcgtaaaaaggactgggacccgaaaaa
gtacggtggcttcgtgagccctacagttgcctattctgtcctagtagt
ggcaaaagttgagaagggaaaatccaagaaactgaagtcagtcaaaga
attattggggataacgattatggagcgctcgtcttttgaaaagaaccc
catcgacttccttgaggcgaaaggttacaaggaagtaaaaaaggatct
cataattaaactaccaaagtatagtctgtttgagttagaaaatggccg
aaaacggatgttggctagcgccagagagcttcaaaaggggaacgaact
cgcactaccgtctaaatacgtgaatttcctgtatttagcgtcccatta
cgagaagttgaaaggttcacctgaagataacgaacagaagcaactttt
tgttgagcagcacaaacattatctcgacgaaatcatagagcaaatttc
ggaattcagtaagagagtcatcctagctgatgccaatctggacaaagt
attaagcgcatacaacaagcacagggataaacccatacgtgagcaggc
ggaaaatattatccatttgtttactcttaccaacctcggcgctccagc
cgcattcaagtattttgacacaacgatagatcgcaaagagtacagatc
taccaaggaggtgctagacgcgacactgattcaccaatccatcacggg
attatatgaaactcggatagatttgtcacagcttgggggtgacggatc
ccccaagaagaagaggaaagtcctcgagggcggaggcgggagcggatc
cccctcccggctccagatgttcttcgctaataaccacgaccaggaatt
tgaccctccaaaggtttacccacctgtcccagctgagaagaggaagcc
catccgggtgctgtctctctttgatggaatcgctacagggctcctggt
gctgaaggacttgggcattcaggtggaccgctacattgcctcggaggt
gtgtgaggactccatcacggtgggcatggtgcggcaccaggggaagat
catgtacgtcggggacgtccgcagcgtcacacagaagcatatccagga
gtggggcccattcgatctggtgattgggggcagtccctgcaatgacct
ctccatcgtcaaccctgctcgcaagggcctctacgagggcactggccg
gctcttctttgagttctaccgcctcctgcatgatgcgcggcccaagga
gggagatgatcgccccttcttctggctctttgcgaatgtggtggccat
gggcgttagtgacaagagggacatctcgcgatttctcgagtccaaccc
tgtgatgattgatgccaaagaagtgtcagctgcacacagggcccgcta
cttctggggtaaccttcccggtatgaacaggccgttggcatccactgt
gaatgataagctggagctgcaggagtgtctggagcatggcaggatagc
caagttcagcaaagtgaggaccattactacgaggtcaaactccataaa
gcagggcaaagaccagcattttcctgtgttcatgaatgagaaagagga
catcttatggtgcactgaaatggaaagggtatttggtttcccagtcca
ctatactgacgtgtccaacatgagccgcttggcgaggcagagactgct
gggccggtcatggagcgtgccagtcatccgccacctcttcgctccgct
gaaggagtattttgcgtgtgtgtccggccggcccggatccggcgcaac
aaacttctctctgctgaaacaagccggagatgtcgaagagaatcctgg
accgaccgagtacaagcccacggtgcgcctcgccacccgcgacgacgt
ccccagggccgtacgcaccctcgccgccgcgttcgccgactaccccgc
cacgcgccacaccgtcgatccggaccgccacatcgagcgggtcaccga
gctgcaagaactcttcctcacgcgcgtcgggctcgacatcggcaaggt
gtgggtcgcggacgacggcgccgcggtggcggtctggaccacgccgga
gagcgtcgaagcgggggcggtgttcgccgagatcggcccgcgcatggc
cgagttgagcggttcccggctggccgcgcagcaacagatggaaggcct
cctggcgccgcaccggcccaaggagcccgcgtggttcctggccaccgt
cggagtctcgcccgaccaccagggcaagggtctgggcagcgccgtcgt
gctccccggagtggaggcggccgagcgcgccggggtgcccgccttcct
ggagacctccgcgccccgcaacctccccttctacgagcggctcggctt
caccgtcaccgccgacgtcgaggtgcccgaaggaccgcgcacctggtg
catgacccgcaagcccggtgcctgaacgcgttaagtcgacaatcaacc
tctggattacaaaatttgtgaaagattgactggtattcttaactatgt
tgctccttttacgctatgtggatacgctgctttaatgcctttgtatca
tgctattgcttcccgtatggctttcattttctcctccttgtataaatc
ctggttgctgtctctttatgaggagttgtggcccgttgtcaggcaacg
tggcgtggtgtgcactgtgtttgctgacgcaacccccactggttgggg
cattgccaccacctgtcagctcctttccgggactttcgctttccccct
ccctattgccacggcggaactcatcgccgcctgccttgcccgctgctg
gacaggggctcggctgttgggcactgacaattccgtggtgttgtcggg
gaaatcatcgtcctttccttggctgctcgcctgtgttgccacctggat
tctgcgcgggacgtccttctgctacgtcccttcggccctcaatccagc
ggaccttccttcccgcggcctgctgccggctctgcggcctcttccgcg
tcttcgccttcgccctcagacgagtcggatctccctttgggccgcctc
cccgcgtcgactttaagaccaatgacttacaaggcagctgtagatctt
agccactttttaaaagaaaaggggggactggaagggctaattcactcc
caacgaagacaagatctgctttttgcttgtactgggtctctctggtta
gaccagatctgagcctgggagctctctggctaactagggaacccactg
cttaagcctcaataaagcttgccttgagtgcttcaagtagtgtgtgcc
cgtctgttgtgtgactctggtaactagagatccctcagacccttttag
tcagtgtggaaaatctctagcagggcccgtttaaacccgctgatcagc
ctcgactgtgccttctagttgccagccatctgttgtttgcccctcccc
cgtgccttccttgaccctggaaggtgccactcccactgtcctttccta
ataaaatgaggaaattgcatcgcattgtctgagtaggtgtcattctat
tctggggggtggggggggcaggacagcaagggggaggattgggaagac
aatagcaggcatgctggggatgcggtgggctctatggcttctgaggcg
gaaagaaccagctggggctctagggggtatccccacgcgccctgtagc
ggcgcattaagcgcggcgggtgtggtggttacgcgcagcgtgaccgct
acacttgccagcgccctagcgcccgctcctttcgctttcttcccttcc
tttctcgccacgttcgccggctttccccgtcaagctctaaatcggggg
ctccctttagggttccgatttagtgctttacggcacctcgaccccaaa
aaacttgattagggtgatggttcacgtagtgggccatcgccctgatag
acggtttttcgccctttgacgttggagtccacgttctttaatagtgga
ctcttgttccaaactggaacaacactcaaccctatctcggtctattct
tttgatttataagggattttgccgatttcggcctattggttaaaaaat
gagctgatttaacaaaaatttaacgcgaattaattctgtggaatgtgt
gtcagttagggtgtggaaagtccccaggctccccagcaggcagaagta
tgcaaagcatgcatctcaattagtcagcaaccaggtgtggaaagtccc
caggctccccagcaggcagaagtatgcaaagcatgcatctcaattagt
cagcaaccatagtcccgcccctaactccgcccatcccgcccctaactc
cgcccagttccgcccattctccgccccatggctgactaatttttttta
tttatgcagaggccgaggccgcctctgcctctgagctattccagaagt
agtgaggaggcttttttggaggcctaggcttttgcaaaaagctcccta
ccgtcgacctctagctagagcttggcgtaatcatggtcatagctgttt
cctgtgtgaaattgttatccgctcacaattccacacaacatacgagcc
ggaagcataaagtgtaaagcctggggtgcctaatgagtgagctaactc
acattaattgcgttgcgctcactgcccgctttccagtcgggaaacctg
tcgtgccagctgcattaatgaatcggccaacgcgcggggagaggcggt
ttgcgtattgggcgctcttccgcttcctcgctcactgactcgctgcgc
tcggtcgttcggctgcggcgagcggtatcagctcactcaaaggcggta
atacggttatccacagaatcaggggataacgcaggaaagaacatgtga
gcaaaaggccagcaaaaggccaggaaccgtaaaaaggccgcgttgctg
gcgtttttccataggctccgcccccctgacgagcatcacaaaaatcga
cgctcaagtcagaggtggcgaaacccgacaggactataaagataccag
gcgtttccccctggaagctccctcgtgcgctctcctgttccgaccctg
ccgcttaccggatacctgtccgcctttctcccttcgggaagcgtggcg
ctttctcatagctcacgctgtaggtatctcagttcggtgtaggtcgtt
cgctccaagctgggctgtgtgcacgaaccccccgttcagcccgaccgc
tgcgccttatccggtaactatcgtcttgagtccaacccggtaagacac
gacttatcgccactggcagcagccactggtaacaggattagcagagcg
aggtatgtaggcggtgctacagagttcttgaagtggtggcctaactac
ggctacactagaagaacagtatttggtatctgcgctctgctgaagcca
gttaccttcggaaaaagagttggtagctcttgatccggcaaacaaacc
accgctggtagcggtggtttttttgtttgcaagcagcagattacgcgc
agaaaaaaaggatctcaagaagatcctttgatcttttctacggggtct
gacgctcagtggaacgaaaactcacgttaagggattttggtcatgaga
ttatcaaaaaggatcttcacctagatccttttaaattaaaaatgaagt
tttaaatcaatctaaagtatatatgagtaaacttggtctgacagttac
caatgcttaatcagtgaggcacctatctcagcgatctgtctatttcgt
tcatccatagttgcctgactccccgtcgtgtagataactacgatacgg
gagggcttaccatctggccccagtgctgcaatgataccgcgagaccca
cgctcaccggctccagatttatcagcaataaaccagccagccggaagg
gccgagcgcagaagtggtcctgcaactttatccgcctccatccagtct
attaattgttgccgggaagctagagtaagtagttcgccagttaatagt
ttgcgcaacgttgttgccattgctacaggcatcgtggtgtcacgctcg
tcgtttggtatggcttcattcagctccggttcccaacgatcaaggcga
gttacatgatcccccatgttgtgcaaaaaagcggttagctccttcggt
cctccgatcgttgtcagaagtaagttggccgcagtgttatcactcatg
gttatggcagcactgcataattctcttactgtcatgccatccgtaaga
tgcttttctgtgactggtgagtactcaaccaagtcattctgagaatag
tgtatgcggcgaccgagttgctcttgcccggcgtcaatacgggataat
accgcgccacatagcagaactttaaaagtgctcatcattggaaaacgt
tcttcggggcgaaaactctcaaggatcttaccgctgttgagatccagt
tcgatgtaacccactcgtgcacccaactgatcttcagcatcttttact
ttcaccagcgtttctgggtgagcaaaaacaggaaggcaaaatgccgca
aaaaagggaataagggcgacacggaaatgttgaatactcatactcttc
ctttttcaatattattgaagcatttatcagggttattgtctcatgagc
ggatacatatttgaatgtatttagaaaaataaacaaataggggttccg
cgcacatttccccgaaaagtgccacctgac.

In an aspect, a disclosed pBK1112 plasmid can comprise the sequence set forth below or a sequence having 70%, 75%, 80%, 85%, 90%, 95%, or more identity to the sequence set forth

(SEQ ID NO: 50)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggtggagtatttacggtaaactgcccacttggcagtacatcaag
tgtatcatatgccaagtacgccccctattgacgtcaatgacggtaaat
ggcccgcctggcattatgcccagtacatgaccttatgggactttccta
cttggcagtacatctacgtattagtcatcgctattaccatggtgatgc
ggttttggcagtacatcaatgggcgtggatagcggtttgactcacggg
gatttccaagtctccaccccattgacgtcaatgggagtttgttttggc
accaaaatcaacgggactttccaaaatgtcgtaacaactccgccccat
tgacgcaaatgggcggtaggcgtgtacggtgggaggtctatataagca
gcgcgttttgcctgtactgggtctctctggttagaccagatctgagcc
tgggagctctctggctaactagggaacccactgcttaagcctcaataa
agcttgccttgagtgcttcaagtagtgtgtgcccgtctgttgtgtgac
tctggtaactagagatccctcagacccttttagtcagtgtggaaaatc
tctagcagtggcgcccgaacagggacttgaaagcgaaagggaaaccag
aggagctctctcgacgcaggactcggcttgctgaagcgcgcacggcaa
gaggcgaggggcggcgactggtgagtacgccaaaaattttgactagcg
gaggctagaaggagagagatgggtgcgagagcgtcagtattaagcggg
ggagaattagatcgcgatgggaaaaaattcggttaaggccagggggaa
agaaaaaatataaattaaaacatatagtatgggcaagcagggagctag
aacgattcgcagttaatcctggcctgttagaaacatcagaaggctgta
gacaaatactgggacagctacaaccatcccttcagacaggatcagaag
aacttagatcattatataatacagtagcaaccctctattgtgtgcatc
aaaggatagagataaaagacaccaaggaagctttagacaagatagagg
aagagcaaaacaaaagtaagaccaccgcacagcaagcggccgctgatc
ttcagacctggaggaggagatatgagggacaattggagaagtgaatta
tataaatataaagtagtaaaaattgaaccattaggagtagcacccacc
aaggcaaagagaagagtggtgcagagagaaaaaagagcagtgggaata
ggagctttgttccttgggttcttgggagcagcaggaagcactatgggc
gcagcgtcaatgacgctgacggtacaggccagacaattattgtctggt
atagtgcagcagcagaacaatttgctgagggctattgaggcgcaacag
catctgttgcaactcacagtctggggcatcaagcagctccaggcaaga
atcctggctgtggaaagatacctaaaggatcaacagctcctggggatt
tggggttgctctggaaaactcatttgcaccactgctgtgccttggaat
gctagttggagtaataaatctctggaacagatttggaatcacacgacc
tggatggagtgggacagagaaattaacaattacacaagcttaatacac
tccttaattgaagaatcgcaaaaccagcaagaaaagaatgaacaagaa
ttattggaattagataaatgggcaagtttgtggaattggtttaacata
acaaattggctgtggtatataaaattattcataatgatagtaggaggc
ttggtaggtttaagaatagtttttgctgtactttctatagtgaataga
gttaggcagggatattcaccattatcgtttcagacccacctcccaacc
ccgaggggacccgacaggcccgaaggaatagaagaagaaggtggagag
agagacagagacagatccattcgattagtgaacggatcggcactgcgt
gcgccaattctgcagacaaatggcagtattcatccacaattttaaaag
aaaaggggggattggggggtacagtgcaggggaaagaatagtagacat
aatagcaacagacatacaaactaaagaattacaaaaacaaattacaaa
aattcaaaattttcgggtttattacagggacagcagagatccagtttg
gttaattaatggggggacgttaacggggcggaacggtaccgagggcct
atttcccatgattccttcatatttgcatatacgatacaaggctgttag
agagataattagaattaatttgactgtaaacacaaagatattagtaca
aaatacgtgacgtagaaagtaataatttcttgggtagtttgcagtttt
aaaattatgttttaaaatggactatcatatgcttaccgtaacttgaaa
gtatttcgatttcttggctttatatatcttgtggaaaggacgaaacac
cgtgtgaagggagaatgaggaagttttagagctagaaatagcaagtta
aaataaggctagtccgttatcaacttgaaaaagtggcaccgagtcggt
gcttttttgaattcgctagctaggtcttgaaaggagtgggaattggct
ccggtgcccgtcagtgggcagagcgcacatcgcccacagtccccgaga
agttggggggaggggtcggcaattgatccggtgcctagagaaggtggc
gcggggtaaactgggaaagtgatgtcgtgtactggctccgcctttttc
ccgaggggggggagaaccgtatataagtgcagtagtcgccgtgaacgt
tctttttcgcaacgggtttgccgccagaacacaggaccggtgccacca
tggactataaggaccacgacggagactacaaggatcatgatattgatt
acaaagacgatgacgataagatggccccaaagaagaagcggaaggtcg
gtatccacggagtcccagcagccgacaagaagtacagcatcggcctgg
ccatcggcaccaactctgtgggctgggccgtgatcaccgacgagtaca
aggtgcccagcaagaaattcaaggtgctgggcaacaccgaccggcaca
gcatcaagaagaacctgatcggagccctgctgttcgacagcggcgaaa
cagccgaggccacccggctgaagagaaccgccagaagaagatacacca
gacggaagaaccggatctgctatctgcaagagatcttcagcaacgaga
tggccaaggtggacgacagcttcttccacagactggaagagtccttcc
tggtggaagaggataagaagcacgagcggcaccccatcttcggcaaca
tcgtggacgaggtggcctaccacgagaagtaccccaccatctaccacc
tgagaaagaaactggtggacagcaccgacaaggccgacctgcggctga
tctatctggccctggcccacatgatcaagttccggggccacttcctga
tcgagggcgacctgaaccccgacaacagcgacgtggacaagctgttca
tccagctggtgcagacctacaaccagctgttcgaggaaaaccccatca
acgccagcggcgtggacgccaaggccatcctgtctgccagactgagca
agagcagacggctggaaaatctgatcgcccagctgcccggcgagaaga
agaatggcctgttcggcaacctgattgccctgagcctgggcctgaccc
ccaacttcaagagcaacttcgacctggccgaggatgccaaactgcagc
tgagcaaggacacctacgacgacgacctggacaacctgctggcccaga
tcggcgaccagtacgccgacctgtttctggccgccaagaacctgtccg
acgccatcctgctgagcgacatcctgagagtgaacaccgagatcacca
aggcccccctgagcgcctctatgatcaagagatacgacgagcaccacc
aggacctgaccctgctgaaagctctcgtgcggcagcagctgcctgaga
agtacaaagagattttcttcgaccagagcaagaacggctacgccggct
acattgacggcggagccagccaggaagagttctacaagttcatcaagc
ccatcctggaaaagatggacggcaccgaggaactgctcgtgaagctga
acagagaggacctgctgcggaagcagcggaccttcgacaacggcagca
tcccccaccagatccacctgggagagctgcacgccattctgcggcggc
aggaagatttttacccattcctgaaggacaaccgggaaaagatcgaga
agatcctgaccttccgcatcccctactacgtgggccctctggccaggg
gaaacagcagattcgcctggatgaccagaaagagcgaggaaaccatca
ccccctggaacttcgaggaagtggtggacaagggcgcttccgcccaga
gcttcatcgagcggatgaccaacttcgataagaacctgcccaacgaga
aggtgctgcccaagcacagcctgctgtacgagtacttcaccgtgtata
acgagctgaccaaagtgaaatacgtgaccgagggaatgagaaagcccg
ccttcctgagcggcgagcagaaaaaggccatcgtggacctgctgttca
agaccaaccggaaagtgaccgtgaagcagctgaaagaggactacttca
agaaaatcgagtgcttcgactccgtggaaatctccggcgtggaagatc
ggttcaacgcctccctgggcacataccacgatctgctgaaaattatca
aggacaaggacttcctggacaatgaggaaaacgaggacattctggaag
atatcgtgctgaccctgacactgtttgaggacagagagatgatcgagg
aacggctgaaaacctatgcccacctgttcgacgacaaagtgatgaagc
agctgaagcggcggagatacaccggctggggcaggctgagccggaagc
tgatcaacggcatccgggacaagcagtccggcaagacaatcctggatt
tcctgaagtccgacggcttcgccaacagaaacttcatgcagctgatcc
acgacgacagcctgacctttaaagaggacatccagaaagcccaggtgt
ccggccagggcgatagcctgcacgagcacattgccaatctggccggca
gccccgccattaagaagggcatcctgcagacagtgaaggtggtggacg
agctcgtgaaagtgatgggccggcacaagcccgagaacatcgtgatcg
aaatggccagagagaaccagaccacccagaagggacagaagaacagcc
gcgagagaatgaagcggatcgaagagggcatcaaagagctgggcagcc
agatcctgaaagaacaccccgtggaaaacacccagctgcagaacgaga
agctgtacctgtactacctgcagaatggggggatatgtacgtggacca
ggaactggacatcaaccggctgtccgactacgatgtggacgctatcgt
gcctcagagctttctgaaggacgactccatcgacaacaaggtgctgac
cagaagcgacaagaaccggggcaagagcgacaacgtgccctccgaaga
ggtcgtgaagaagatgaagaactactggcggcagctgctgaacgccaa
gctgattacccagagaaagttcgacaatctgaccaaggccgagagagg
cggcctgagcgaactggataaggccggcttcatcaagagacagctggt
ggaaacccggcagatcacaaagcacgtggcacagatactagattcccg
aatgaatacgaaatacgacgagaacgataagctgattcgggaagtcaa
agtaatcactttaaagtcaaaattggtgtcggacttcagaaaggattt
tcaattctataaagttagggagataaataactaccaccatgcgcacga
cgcttatcttaatgccgtcgtagggaccgcactcattaagaaataccc
gaagctagaaagtgagtttgtgtatggtgattacaaagtttatgacgt
ccgtaagatgatcgcgaaaagcgaacaggagataggcaaggctacagc
caaatacttcttttattctaacattatgaatttctttaagacggaaat
cactctggcaaacggagagatacgcaaacgacctttaattgaaaccaa
tggggagacaggtgaaatcgtatgggataagggccgggacttcgcgac
ggtgagaaaagttttgtccatgccccaagtcaacatagtaaagaaaac
tgaggtgcagaccggagggttttcaaaggaatcgattcttccaaaaag
gaatagtgataagctcatcgctcgtaaaaaggactgggacccgaaaaa
gtacggtggcttcgtgagccctacagttgcctattctgtcctagtagt
ggcaaaagttgagaagggaaaatccaagaaactgaagtcagtcaaaga
attattggggataacgattatggagcgctcgtcttttgaaaagaaccc
catcgacttccttgaggcgaaaggttacaaggaagtaaaaaaggatct
cataattaaactaccaaagtatagtctgtttgagttagaaaatggccg
aaaacggatgttggctagcgccagagagcttcaaaaggggaacgaact
cgcactaccgtctaaatacgtgaatttcctgtatttagcgtcccatta
cgagaagttgaaaggttcacctgaagataacgaacagaagcaactttt
tgttgagcagcacaaacattatctcgacgaaatcatagagcaaatttc
ggaattcagtaagagagtcatcctagctgatgccaatctggacaaagt
attaagcgcatacaacaagcacagggataaacccatacgtgagcaggc
ggaaaatattatccatttgtttactcttaccaacctcggcgctccagc
cgcattcaagtattttgacacaacgatagatcgcaaagagtacagatc
taccaaggaggtgctagacgcgacactgattcaccaatccatcacggg
attatatgaaactcggatagatttgtcacagcttgggggtgacggatc
ccccaagaagaagaggaaagtcctcgagggcggaggcgggagcggatc
cccctcccggctccagatgttcttcgctaataaccacgaccaggaatt
tgaccctccaaaggtttacccacctgtcccagctgagaagaggaagcc
catccgggtgctgtctctctttgatggaatcgctacagggctcctggt
gctgaaggacttgggcattcaggtggaccgctacattgcctcggaggt
gtgtgaggactccatcacggtgggcatggtgcggcaccaggggaagat
catgtacgtcggggacgtccgcagcgtcacacagaagcatatccagga
gtggggcccattcgatctggtgattgggggcagtccctgcaatgacct
ctccatcgtcaaccctgctcgcaagggcctctacgagggcactggccg
gctcttctttgagttctaccgcctcctgcatgatgcgcggcccaagga
gggagatgatcgccccttcttctggctctttgcgaatgtggtggccat
gggcgttagtgacaagagggacatctcgcgatttctcgagtccaaccc
tgtgatgattgatgccaaagaagtgtcagctgcacacagggcccgcta
cttctggggtaaccttcccggtatgaacaggccgttggcatccactgt
gaatgataagctggagctgcaggagtgtctggagcatggcaggatagc
caagttcagcaaagtgaggaccattactacgaggtcaaactccataaa
gcagggcaaagaccagcattttcctgtgttcatgaatgagaaagagga
catcttatggtgcactgaaatggaaagggtatttggtttcccagtcca
ctatactgacgtgtccaacatgagccgcttggcgaggcagagactgct
gggccggtcatggagcgtgccagtcatccgccacctcttcgctccgct
gaaggagtattttgcgtgtgtgtccggccggcccggatccggcgcaac
aaacttctctctgctgaaacaagccggagatgtcgaagagaatcctgg
accgaccgagtacaagcccacggtgcgcctcgccacccgcgacgacgt
ccccagggccgtacgcaccctcgccgccgcgttcgccgactaccccgc
cacgcgccacaccgtcgatccggaccgccacatcgagcgggtcaccga
gctgcaagaactcttcctcacgcgcgtcgggctcgacatcggcaaggt
gtgggtcgcggacgacggcgccgcggtggcggtctggaccacgccgga
gagcgtcgaagcgggggcggtgttcgccgagatcggcccgcgcatggc
cgagttgagcggttcccggctggccgcgcagcaacagatggaaggcct
cctggcgccgcaccggcccaaggagcccgcgtggttcctggccaccgt
cggagtctcgcccgaccaccagggcaagggtctgggcagcgccgtcgt
gctccccggagtggaggcggccgagcgcgccggggtgcccgccttcct
ggagacctccgcgccccgcaacctccccttctacgagcggctcggctt
caccgtcaccgccgacgtcgaggtgcccgaaggaccgcgcacctggtg
catgacccgcaagcccggtgcctgaacgcgttaagtcgacaatcaacc
tctggattacaaaatttgtgaaagattgactggtattcttaactatgt
tgctccttttacgctatgtggatacgctgctttaatgcctttgtatca
tgctattgcttcccgtatggctttcattttctcctccttgtataaatc
ctggttgctgtctctttatgaggagttgtggcccgttgtcaggcaacg
tggcgtggtgtgcactgtgtttgctgacgcaacccccactggttgggg
cattgccaccacctgtcagctcctttccgggactttcgctttccccct
ccctattgccacggcggaactcatcgccgcctgccttgcccgctgctg
gacaggggctcggctgttgggcactgacaattccgtggtgttgtcggg
gaaatcatcgtcctttccttggctgctcgcctgtgttgccacctggat
tctgcgcgggacgtccttctgctacgtcccttcggccctcaatccagc
ggaccttccttcccgcggcctgctgccggctctgcggcctcttccgcg
tcttcgccttcgccctcagacgagtcggatctccctttgggccgcctc
cccgcgtcgactttaagaccaatgacttacaaggcagctgtagatctt
agccactttttaaaagaaaaggggggactggaagggctaattcactcc
caacgaagacaagatctgctttttgcttgtactgggtctctctggtta
gaccagatctgagcctgggagctctctggctaactagggaacccactg
cttaagcctcaataaagcttgccttgagtgcttcaagtagtgtgtgcc
cgtctgttgtgtgactctggtaactagagatccctcagacccttttag
tcagtgtggaaaatctctagcagggcccgtttaaacccgctgatcagc
ctcgactgtgccttctagttgccagccatctgttgtttgcccctcccc
cgtgccttccttgaccctggaaggtgccactcccactgtcctttccta
ataaaatgaggaaattgcatcgcattgtctgagtaggtgtcattctat
tctggggggtggggggggcaggacagcaagggggaggattgggaagac
aatagcaggcatgctggggatgcggtgggctctatggcttctgaggcg
gaaagaaccagctggggctctagggggtatccccacgcgccctgtagc
ggcgcattaagcgcggcgggtgtggtggttacgcgcagcgtgaccgct
acacttgccagcgccctagcgcccgctcctttcgctttcttcccttcc
tttctcgccacgttcgccggctttccccgtcaagctctaaatcggggg
ctccctttagggttccgatttagtgctttacggcacctcgaccccaaa
aaacttgattagggtgatggttcacgtagtgggccatcgccctgatag
acggtttttcgccctttgacgttggagtccacgttctttaatagtgga
ctcttgttccaaactggaacaacactcaaccctatctcggtctattct
tttgatttataagggattttgccgatttcggcctattggttaaaaaat
gagctgatttaacaaaaatttaacgcgaattaattctgtggaatgtgt
gtcagttagggtgtggaaagtccccaggctccccagcaggcagaagta
tgcaaagcatgcatctcaattagtcagcaaccaggtgtggaaagtccc
caggctccccagcaggcagaagtatgcaaagcatgcatctcaattagt
cagcaaccatagtcccgcccctaactccgcccatcccgcccctaactc
cgcccagttccgcccattctccgccccatggctgactaatttttttta
tttatgcagaggccgaggccgcctctgcctctgagctattccagaagt
agtgaggaggcttttttggaggcctaggcttttgcaaaaagctcccta
ccgtcgacctctagctagagcttggcgtaatcatggtcatagctgttt
cctgtgtgaaattgttatccgctcacaattccacacaacatacgagcc
ggaagcataaagtgtaaagcctggggtgcctaatgagtgagctaactc
acattaattgcgttgcgctcactgcccgctttccagtcgggaaacctg
tcgtgccagctgcattaatgaatcggccaacgcgcggggagaggcggt
ttgcgtattgggcgctcttccgcttcctcgctcactgactcgctgcgc
tcggtcgttcggctgcggcgagcggtatcagctcactcaaaggcggta
atacggttatccacagaatcaggggataacgcaggaaagaacatgtga
gcaaaaggccagcaaaaggccaggaaccgtaaaaaggccgcgttgctg
gcgtttttccataggctccgcccccctgacgagcatcacaaaaatcga
cgctcaagtcagaggtggcgaaacccgacaggactataaagataccag
gcgtttccccctggaagctccctcgtgcgctctcctgttccgaccctg
ccgcttaccggatacctgtccgcctttctcccttcgggaagcgtggcg
ctttctcatagctcacgctgtaggtatctcagttcggtgtaggtcgtt
cgctccaagctgggctgtgtgcacgaaccccccgttcagcccgaccgc
tgcgccttatccggtaactatcgtcttgagtccaacccggtaagacac
gacttatcgccactggcagcagccactggtaacaggattagcagagcg
aggtatgtaggcggtgctacagagttcttgaagtggtggcctaactac
ggctacactagaagaacagtatttggtatctgcgctctgctgaagcca
gttaccttcggaaaaagagttggtagctcttgatccggcaaacaaacc
accgctggtagcggtggtttttttgtttgcaagcagcagattacgcgc
agaaaaaaaggatctcaagaagatcctttgatcttttctacggggtct
gacgctcagtggaacgaaaactcacgttaagggattttggtcatgaga
ttatcaaaaaggatcttcacctagatccttttaaattaaaaatgaagt
tttaaatcaatctaaagtatatatgagtaaacttggtctgacagttac
caatgcttaatcagtgaggcacctatctcagcgatctgtctatttcgt
tcatccatagttgcctgactccccgtcgtgtagataactacgatacgg
gagggcttaccatctggccccagtgctgcaatgataccgcgagaccca
cgctcaccggctccagatttatcagcaataaaccagccagccggaagg
gccgagcgcagaagtggtcctgcaactttatccgcctccatccagtct
attaattgttgccgggaagctagagtaagtagttcgccagttaatagt
ttgcgcaacgttgttgccattgctacaggcatcgtggtgtcacgctcg
tcgtttggtatggcttcattcagctccggttcccaacgatcaaggcga
gttacatgatcccccatgttgtgcaaaaaagcggttagctccttcggt
cctccgatcgttgtcagaagtaagttggccgcagtgttatcactcatg
gttatggcagcactgcataattctcttactgtcatgccatccgtaaga
tgcttttctgtgactggtgagtactcaaccaagtcattctgagaatag
tgtatgcggcgaccgagttgctcttgcccggcgtcaatacgggataat
accgcgccacatagcagaactttaaaagtgctcatcattggaaaacgt
tcttcggggcgaaaactctcaaggatcttaccgctgttgagatccagt
tcgatgtaacccactcgtgcacccaactgatcttcagcatcttttact
ttcaccagcgtttctgggtgagcaaaaacaggaaggcaaaatgccgca
aaaaagggaataagggcgacacggaaatgttgaatactcatactcttc
ctttttcaatattattgaagcatttatcagggttattgtctcatgagc
ggatacatatttgaatgtatttagaaaaataaacaaataggggttccg
cgcacatttccccgaaaagtgccacctgac.

In an aspect, a disclosed pBK1426 plasmid can comprise the sequence set forth below or a sequence having 70%, 75%, 80%, 85%, 90%, 95%, or more identity to the sequence set forth

(SEQ ID NO: 53)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggtggagtatttacggtaaactgcccacttggcagtacatcaag
tgtatcatatgccaagtacgccccctattgacgtcaatgacggtaaat
ggcccgcctggcattatgcccagtacatgaccttatgggactttccta
cttggcagtacatctacgtattagtcatcgctattaccatggtgatgc
ggttttggcagtacatcaatgggcgtggatagcggtttgactcacggg
gatttccaagtctccaccccattgacgtcaatgggagtttgttttggc
accaaaatcaacgggactttccaaaatgtcgtaacaactccgccccat
tgacgcaaatgggcggtaggcgtgtacggtgggaggtctatataagca
gcgcgttttgcctgtactgggtctctctggttagaccagatctgagcc
tgggagctctctggctaactagggaacccactgcttaagcctcaataa
agcttgccttgagtgcttcaagtagtgtgtgcccgtctgttgtgtgac
tctggtaactagagatccctcagacccttttagtcagtgtggaaaatc
tctagcagtggcgcccgaacagggacttgaaagcgaaagggaaaccag
aggagctctctcgacgcaggactcggcttgctgaagcgcgcacggcaa
gaggcgaggggcggcgactggtgagtacgccaaaaattttgactagcg
gaggctagaaggagagagatgggtgcgagagcgtcagtattaagcggg
ggagaattagatcgcgatgggaaaaaattcggttaaggccagggggaa
agaaaaaatataaattaaaacatatagtatgggcaagcagggagctag
aacgattcgcagttaatcctggcctgttagaaacatcagaaggctgta
gacaaatactgggacagctacaaccatcccttcagacaggatcagaag
aacttagatcattatataatacagtagcaaccctctattgtgtgcatc
aaaggatagagataaaagacaccaaggaagctttagacaagatagagg
aagagcaaaacaaaagtaagaccaccgcacagcaagcggccgctgatc
ttcagacctggaggaggagatatgagggacaattggagaagtgaatta
tataaatataaagtagtaaaaattgaaccattaggagtagcacccacc
aaggcaaagagaagagtggtgcagagagaaaaaagagcagtgggaata
ggagctttgttccttgggttcttgggagcagcaggaagcactatgggc
gcagcgtcaatgacgctgacggtacaggccagacaattattgtctggt
atagtgcagcagcagaacaatttgctgagggctattgaggcgcaacag
catctgttgcaactcacagtctggggcatcaagcagctccaggcaaga
atcctggctgtggaaagatacctaaaggatcaacagctcctggggatt
tggggttgctctggaaaactcatttgcaccactgctgtgccttggaat
gctagttggagtaataaatctctggaacagatttggaatcacacgacc
tggatggagtgggacagagaaattaacaattacacaagcttaatacac
tccttaattgaagaatcgcaaaaccagcaagaaaagaatgaacaagaa
ttattggaattagataaatgggcaagtttgtggaattggtttaacata
acaaattggctgtggtatataaaattattcataatgatagtaggaggc
ttggtaggtttaagaatagtttttgctgtactttctatagtgaataga
gttaggcagggatattcaccattatcgtttcagacccacctcccaacc
ccgaggggacccgacaggcccgaaggaatagaagaagaaggtggagag
agagacagagacagatccattcgattagtgaacggatcggcactgcgt
gcgccaattctgcagacaaatggcagtattcatccacaattttaaaag
aaaaggggggattggggggtacagtgcaggggaaagaatagtagacat
aatagcaacagacatacaaactaaagaattacaaaaacaaattacaaa
aattcaaaattttcgggtttattacagggacagcagagatccagtttg
gttaattaatggggggacgttaacggggcggaacggtaccgagggcct
atttcccatgattccttcatatttgcatatacgatacaaggctgttag
agagataattagaattaatttgactgtaaacacaaagatattagtaca
aaatacgtgacgtagaaagtaataatttcttgggtagtttgcagtttt
aaaattatgttttaaaatggactatcatatgcttaccgtaacttgaaa
gtatttcgatttcttggctttatatatcttgtggaaaggacgaaacac
cggcggtactgcaccaggcggcgttttagagctagaaatagcaagtta
aaataaggctagtccgttatcaacttgaaaaagtggcaccgagtcggt
gcttttttgaattcgctagctaggtcttgaaaggagtgggaattggct
ccggtgcccgtcagtgggcagagcgcacatcgcccacagtccccgaga
agttggggggaggggtcggcaattgatccggtgcctagagaaggtggc
gcggggtaaactgggaaagtgatgtcgtgtactggctccgcctttttc
ccgaggggggggagaaccgtatataagtgcagtagtcgccgtgaacgt
tctttttcgcaacgggtttgccgccagaacacaggaccggtgccacca
tggactataaggaccacgacggagactacaaggatcatgatattgatt
acaaagacgatgacgataagatggccccaaagaagaagcggaaggtcg
gtatccacggagtcccagcagccgacaagaagtacagcatcggcctgg
ccatcggcaccaactctgtgggctgggccgtgatcaccgacgagtaca
aggtgcccagcaagaaattcaaggtgctgggcaacaccgaccggcaca
gcatcaagaagaacctgatcggagccctgctgttcgacagcggcgaaa
cagccgaggccacccggctgaagagaaccgccagaagaagatacacca
gacggaagaaccggatctgctatctgcaagagatcttcagcaacgaga
tggccaaggtggacgacagcttcttccacagactggaagagtccttcc
tggtggaagaggataagaagcacgagcggcaccccatcttcggcaaca
tcgtggacgaggtggcctaccacgagaagtaccccaccatctaccacc
tgagaaagaaactggtggacagcaccgacaaggccgacctgcggctga
tctatctggccctggcccacatgatcaagttccggggccacttcctga
tcgagggcgacctgaaccccgacaacagcgacgtggacaagctgttca
tccagctggtgcagacctacaaccagctgttcgaggaaaaccccatca
acgccagcggcgtggacgccaaggccatcctgtctgccagactgagca
agagcagacggctggaaaatctgatcgcccagctgcccggcgagaaga
agaatggcctgttcggcaacctgattgccctgagcctgggcctgaccc
ccaacttcaagagcaacttcgacctggccgaggatgccaaactgcagc
tgagcaaggacacctacgacgacgacctggacaacctgctggcccaga
tcggcgaccagtacgccgacctgtttctggccgccaagaacctgtccg
acgccatcctgctgagcgacatcctgagagtgaacaccgagatcacca
aggcccccctgagcgcctctatgatcaagagatacgacgagcaccacc
aggacctgaccctgctgaaagctctcgtgcggcagcagctgcctgaga
agtacaaagagattttcttcgaccagagcaagaacggctacgccggct
acattgacggcggagccagccaggaagagttctacaagttcatcaagc
ccatcctggaaaagatggacggcaccgaggaactgctcgtgaagctga
acagagaggacctgctgcggaagcagcggaccttcgacaacggcagca
tcccccaccagatccacctgggagagctgcacgccattctgcggcggc
aggaagatttttacccattcctgaaggacaaccgggaaaagatcgaga
agatcctgaccttccgcatcccctactacgtgggccctctggccaggg
gaaacagcagattcgcctggatgaccagaaagagcgaggaaaccatca
ccccctggaacttcgaggaagtggtggacaagggcgcttccgcccaga
gcttcatcgagcggatgaccaacttcgataagaacctgcccaacgaga
aggtgctgcccaagcacagcctgctgtacgagtacttcaccgtgtata
acgagctgaccaaagtgaaatacgtgaccgagggaatgagaaagcccg
ccttcctgagcggcgagcagaaaaaggccatcgtggacctgctgttca
agaccaaccggaaagtgaccgtgaagcagctgaaagaggactacttca
agaaaatcgagtgcttcgactccgtggaaatctccggcgtggaagatc
ggttcaacgcctccctgggcacataccacgatctgctgaaaattatca
aggacaaggacttcctggacaatgaggaaaacgaggacattctggaag
atatcgtgctgaccctgacactgtttgaggacagagagatgatcgagg
aacggctgaaaacctatgcccacctgttcgacgacaaagtgatgaagc
agctgaagcggcggagatacaccggctggggcaggctgagccggaagc
tgatcaacggcatccgggacaagcagtccggcaagacaatcctggatt
tcctgaagtccgacggcttcgccaacagaaacttcatgcagctgatcc
acgacgacagcctgacctttaaagaggacatccagaaagcccaggtgt
ccggccagggcgatagcctgcacgagcacattgccaatctggccggca
gccccgccattaagaagggcatcctgcagacagtgaaggtggtggacg
agctcgtgaaagtgatgggccggcacaagcccgagaacatcgtgatcg
aaatggccagagagaaccagaccacccagaagggacagaagaacagcc
gcgagagaatgaagcggatcgaagagggcatcaaagagctgggcagcc
agatcctgaaagaacaccccgtggaaaacacccagctgcagaacgaga
agctgtacctgtactacctgcagaatggggggatatgtacgtggacca
ggaactggacatcaaccggctgtccgactacgatgtggacgctatcgt
gcctcagagctttctgaaggacgactccatcgacaacaaggtgctgac
cagaagcgacaagaaccggggcaagagcgacaacgtgccctccgaaga
ggtcgtgaagaagatgaagaactactggcggcagctgctgaacgccaa
gctgattacccagagaaagttcgacaatctgaccaaggccgagagagg
cggcctgagcgaactggataaggccggcttcatcaagagacagctggt
ggaaacccggcagatcacaaagcacgtggcacagatactagattcccg
aatgaatacgaaatacgacgagaacgataagctgattcgggaagtcaa
agtaatcactttaaagtcaaaattggtgtcggacttcagaaaggattt
tcaattctataaagttagggagataaataactaccaccatgcgcacga
cgcttatcttaatgccgtcgtagggaccgcactcattaagaaataccc
gaagctagaaagtgagtttgtgtatggtgattacaaagtttatgacgt
ccgtaagatgatcgcgaaaagcgaacaggagataggcaaggctacagc
caaatacttcttttattctaacattatgaatttctttaagacggaaat
cactctggcaaacggagagatacgcaaacgacctttaattgaaaccaa
tggggagacaggtgaaatcgtatgggataagggccgggacttcgcgac
ggtgagaaaagttttgtccatgccccaagtcaacatagtaaagaaaac
tgaggtgcagaccggagggttttcaaaggaatcgattcttccaaaaag
gaatagtgataagctcatcgctcgtaaaaaggactgggacccgaaaaa
gtacggtggcttcgtgagccctacagttgcctattctgtcctagtagt
ggcaaaagttgagaagggaaaatccaagaaactgaagtcagtcaaaga
attattggggataacgattatggagcgctcgtcttttgaaaagaaccc
catcgacttccttgaggcgaaaggttacaaggaagtaaaaaaggatct
cataattaaactaccaaagtatagtctgtttgagttagaaaatggccg
aaaacggatgttggctagcgccagagagcttcaaaaggggaacgaact
cgcactaccgtctaaatacgtgaatttcctgtatttagcgtcccatta
cgagaagttgaaaggttcacctgaagataacgaacagaagcaactttt
tgttgagcagcacaaacattatctcgacgaaatcatagagcaaatttc
ggaattcagtaagagagtcatcctagctgatgccaatctggacaaagt
attaagcgcatacaacaagcacagggataaacccatacgtgagcaggc
ggaaaatattatccatttgtttactcttaccaacctcggcgctccagc
cgcattcaagtattttgacacaacgatagatcgcaaagagtacagatc
taccaaggaggtgctagacgcgacactgattcaccaatccatcacggg
attatatgaaactcggatagatttgtcacagcttgggggtgacggatc
ccccaagaagaagaggaaagtcctcgagggcggaggcgggagcggatc
cccctcccggctccagatgttcttcgctaataaccacgaccaggaatt
tgaccctccaaaggtttacccacctgtcccagctgagaagaggaagcc
catccgggtgctgtctctctttgatggaatcgctacagggctcctggt
gctgaaggacttgggcattcaggtggaccgctacattgcctcggaggt
gtgtgaggactccatcacggtgggcatggtgcggcaccaggggaagat
catgtacgtcggggacgtccgcagcgtcacacagaagcatatccagga
gtggggcccattcgatctggtgattgggggcagtccctgcaatgacct
ctccatcgtcaaccctgctcgcaagggcctctacgagggcactggccg
gctcttctttgagttctaccgcctcctgcatgatgcgcggcccaagga
gggagatgatcgccccttcttctggctctttgagaatgtggtggccat
gggcgttagtgacaagagggacatctcgcgatttctcgagtccaaccc
tgtgatgattgatgccaaagaagtgtcagctgcacacagggcccgcta
cttctggggtaaccttcccggtatgaacaggccgttggcatccactgt
gaatgataagctggagctgcaggagtgtctggagcatggcaggatagc
caagttcagcaaagtgaggaccattactacgaggtcaaactccataaa
gcagggcaaagaccagcattttcctgtgttcatgaatgagaaagagga
catcttatggtgcactgaaatggaaagggtatttggtttcccagtcca
ctatactgacgtgtccaacatgagccgcttggcgaggcagagactgct
gggccggtcatggagcgtgccagtcatccgccacctcttcgctccgct
gaaggagtattttgcgtgtgtgtccggccggcccggatccggcgcaac
aaacttctctctgctgaaacaagccggagatgtcgaagagaatcctgg
accgaccgagtacaagcccacggtgcgcctcgccacccgcgacgacgt
ccccagggccgtacgcaccctcgccgccgcgttcgccgactaccccgc
cacgcgccacaccgtcgatccggaccgccacatcgagcgggtcaccga
gctgcaagaactcttcctcacgcgcgtcgggctcgacatcggcaaggt
gtgggtcgcggacgacggcgccgcggtggcggtctggaccacgccgga
gagcgtcgaagcgggggcggtgttcgccgagatcggcccgcgcatggc
cgagttgagcggttcccggctggccgcgcagcaacagatggaaggcct
cctggcgccgcaccggcccaaggagcccgcgtggttcctggccaccgt
cggagtctcgcccgaccaccagggcaagggtctgggcagcgccgtcgt
gctccccggagtggaggcggccgagcgcgccggggtgcccgccttcct
ggagacctccgcgccccgcaacctccccttctacgagcggctcggctt
caccgtcaccgccgacgtcgaggtgcccgaaggaccgcgcacctggtg
catgacccgcaagcccggtgcctgaacgcgttaagtcgacaatcaacc
tctggattacaaaatttgtgaaagattgactggtattcttaactatgt
tgctccttttacgctatgtggatacgctgctttaatgcctttgtatca
tgctattgcttcccgtatggctttcattttctcctccttgtataaatc
ctggttgctgtctctttatgaggagttgtggcccgttgtcaggcaacg
tggcgtggtgtgcactgtgtttgctgacgcaacccccactggttgggg
cattgccaccacctgtcagctcctttccgggactttcgctttccccct
ccctattgccacggcggaactcatcgccgcctgccttgcccgctgctg
gacaggggctcggctgttgggcactgacaattccgtggtgttgtcggg
gaaatcatcgtcctttccttggctgctcgcctgtgttgccacctggat
tctgcgcgggacgtccttctgctacgtcccttcggccctcaatccagc
ggaccttccttcccgcggcctgctgccggctctgcggcctcttccgcg
tcttcgccttcgccctcagacgagtcggatctccctttgggccgcctc
cccgcgtcgactttaagaccaatgacttacaaggcagctgtagatctt
agccactttttaaaagaaaaggggggactggaagggctaattcactcc
caacgaagacaagatctgctttttgcttgtactgggtctctctggtta
gaccagatctgagcctgggagctctctggctaactagggaacccactg
cttaagcctcaataaagcttgccttgagtgcttcaagtagtgtgtgcc
cgtctgttgtgtgactctggtaactagagatccctcagacccttttag
tcagtgtggaaaatctctagcagggcccgtttaaacccgctgatcagc
ctcgactgtgccttctagttgccagccatctgttgtttgcccctcccc
cgtgccttccttgaccctggaaggtgccactcccactgtcctttccta
ataaaatgaggaaattgcatcgcattgtctgagtaggtgtcattctat
tctggggggtggggggggcaggacagcaagggggaggattgggaagac
aatagcaggcatgctggggatgcggtgggctctatggcttctgaggcg
gaaagaaccagctggggctctagggggtatccccacgcgccctgtagc
ggcgcattaagcgcggcgggtgtggtggttacgcgcagcgtgaccgct
acacttgccagcgccctagcgcccgctcctttcgctttcttcccttcc
tttctcgccacgttcgccggctttccccgtcaagctctaaatcggggg
ctccctttagggttccgatttagtgctttacggcacctcgaccccaaa
aaacttgattagggtgatggttcacgtagtgggccatcgccctgatag
acggtttttcgccctttgacgttggagtccacgttctttaatagtgga
ctcttgttccaaactggaacaacactcaaccctatctcggtctattct
tttgatttataagggattttgccgatttcggcctattggttaaaaaat
gagctgatttaacaaaaatttaacgcgaattaattctgtggaatgtgt
gtcagttagggtgtggaaagtccccaggctccccagcaggcagaagta
tgcaaagcatgcatctcaattagtcagcaaccaggtgtggaaagtccc
caggctccccagcaggcagaagtatgcaaagcatgcatctcaattagt
cagcaaccatagtcccgcccctaactccgcccatcccgcccctaactc
cgcccagttccgcccattctccgccccatggctgactaatttttttta
tttatgcagaggccgaggccgcctctgcctctgagctattccagaagt
agtgaggaggcttttttggaggcctaggcttttgcaaaaagctcccta
ccgtcgacctctagctagagcttggcgtaatcatggtcatagctgttt
cctgtgtgaaattgttatccgctcacaattccacacaacatacgagcc
ggaagcataaagtgtaaagcctggggtgcctaatgagtgagctaactc
acattaattgcgttgcgctcactgcccgctttccagtcgggaaacctg
tcgtgccagctgcattaatgaatcggccaacgcgcggggagaggcggt
ttgcgtattgggcgctcttccgcttcctcgctcactgactcgctgcgc
tcggtcgttcggctgcggcgagcggtatcagctcactcaaaggcggta
atacggttatccacagaatcaggggataacgcaggaaagaacatgtga
gcaaaaggccagcaaaaggccaggaaccgtaaaaaggccgcgttgctg
gcgtttttccataggctccgcccccctgacgagcatcacaaaaatcga
cgctcaagtcagaggtggcgaaacccgacaggactataaagataccag
gcgtttccccctggaagctccctcgtgcgctctcctgttccgaccctg
ccgcttaccggatacctgtccgcctttctcccttcgggaagcgtggcg
ctttctcatagctcacgctgtaggtatctcagttcggtgtaggtcgtt
cgctccaagctgggctgtgtgcacgaaccccccgttcagcccgaccgc
tgcgccttatccggtaactatcgtcttgagtccaacccggtaagacac
gacttatcgccactggcagcagccactggtaacaggattagcagagcg
aggtatgtaggcggtgctacagagttcttgaagtggtggcctaactac
ggctacactagaagaacagtatttggtatctgcgctctgctgaagcca
gttaccttcggaaaaagagttggtagctcttgatccggcaaacaaacc
accgctggtagcggtggtttttttgtttgcaagcagcagattacgcgc
agaaaaaaaggatctcaagaagatcctttgatcttttctacggggtct
gacgctcagtggaacgaaaactcacgttaagggattttggtcatgaga
ttatcaaaaaggatcttcacctagatccttttaaattaaaaatgaagt
tttaaatcaatctaaagtatatatgagtaaacttggtctgacagttac
caatgcttaatcagtgaggcacctatctcagcgatctgtctatttcgt
tcatccatagttgcctgactccccgtcgtgtagataactacgatacgg
gagggcttaccatctggccccagtgctgcaatgataccgcgagaccca
cgctcaccggctccagatttatcagcaataaaccagccagccggaagg
gccgagcgcagaagtggtcctgcaactttatccgcctccatccagtct
attaattgttgccgggaagctagagtaagtagttcgccagttaatagt
ttgcgcaacgttgttgccattgctacaggcatcgtggtgtcacgctcg
tcgtttggtatggcttcattcagctccggttcccaacgatcaaggcga
gttacatgatcccccatgttgtgcaaaaaagcggttagctccttcggt
cctccgatcgttgtcagaagtaagttggccgcagtgttatcactcatg
gttatggcagcactgcataattctcttactgtcatgccatccgtaaga
tgcttttctgtgactggtgagtactcaaccaagtcattctgagaatag
tgtatgcggcgaccgagttgctcttgcccggcgtcaatacgggataat
accgcgccacatagcagaactttaaaagtgctcatcattggaaaacgt
tcttcggggcgaaaactctcaaggatcttaccgctgttgagatccagt
tcgatgtaacccactcgtgcacccaactgatcttcagcatcttttact
ttcaccagcgtttctgggtgagcaaaaacaggaaggcaaaatgccgca
aaaaagggaataagggcgacacggaaatgttgaatactcatactcttc
ctttttcaatattattgaagcatttatcagggttattgtctcatgagc
ggatacatatttgaatgtatttagaaaaataaacaaataggggttccg
cgcacatttccccgaaaagtgccacctgac.

In an aspect, a disclosed pBK1427 plasmid can comprise the sequence set forth below or a sequence having 70%, 75%, 80%, 85%, 90%, 95%, or more identity to the sequence set forth

(SEQ ID NO: 54)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggtggagtatttacggtaaactgcccacttggcagtacatcaag
tgtatcatatgccaagtacgccccctattgacgtcaatgacggtaaat
ggcccgcctggcattatgcccagtacatgaccttatgggactttccta
cttggcagtacatctacgtattagtcatcgctattaccatggtgatgc
ggttttggcagtacatcaatgggcgtggatagcggtttgactcacggg
gatttccaagtctccaccccattgacgtcaatgggagtttgttttggc
accaaaatcaacgggactttccaaaatgtcgtaacaactccgccccat
tgacgcaaatgggcggtaggcgtgtacggtgggaggtctatataagca
gcgcgttttgcctgtactgggtctctctggttagaccagatctgagcc
tgggagctctctggctaactagggaacccactgcttaagcctcaataa
agcttgccttgagtgcttcaagtagtgtgtgcccgtctgttgtgtgac
tctggtaactagagatccctcagacccttttagtcagtgtggaaaatc
tctagcagtggcgcccgaacagggacttgaaagcgaaagggaaaccag
aggagctctctcgacgcaggactcggcttgctgaagcgcgcacggcaa
gaggcgaggggcggcgactggtgagtacgccaaaaattttgactagcg
gaggctagaaggagagagatgggtgcgagagcgtcagtattaagcggg
ggagaattagatcgcgatgggaaaaaattcggttaaggccagggggaa
agaaaaaatataaattaaaacatatagtatgggcaagcagggagctag
aacgattcgcagttaatcctggcctgttagaaacatcagaaggctgta
gacaaatactgggacagctacaaccatcccttcagacaggatcagaag
aacttagatcattatataatacagtagcaaccctctattgtgtgcatc
aaaggatagagataaaagacaccaaggaagctttagacaagatagagg
aagagcaaaaaaaagtaagaccaccgcacagcaagcggccgctgatct
tcagacctggaggaggagatatgagggacaattggagaagtgaattat
ataaatataaagtagtaaaaattgaaccattaggagtagcacccacca
aggcaaagagaagagtggtgcagagagaaaaaagagcagtgggaatag
gagctttgttccttgggttcttgggagcagcaggaagcactatgggcg
cagcgtcaatgacgctgacggtacaggccagacaattattgtctggta
tagtgcagcagcagaacaatttgctgagggctattgaggcgcaacagc
atctgttgcaactcacagtctggggcatcaagcagctccaggcaagaa
tcctggctgtggaaagatacctaaaggatcaacagctcctggggattt
ggggttgctctggaaaactcatttgcaccactgctgtgccttggaatg
ctagttggagtaataaatctctggaacagatttggaatcacacgacct
ggatggagtgggacagagaaattaacaattacacaagcttaatacact
ccttaattgaagaatcgcaaaaccagcaagaaaagaatgaacaagaat
tattggaattagataaatgggcaagtttgtggaattggtttaacataa
caaattggctgtggtatataaaattattcataatgatagtaggaggct
tggtaggtttaagaatagtttttgctgtactttctatagtgaatagag
ttaggcagggatattcaccattatcgtttcagacccacctcccaaccc
cgaggggacccgacaggcccgaaggaatagaagaagaaggtggagaga
gagacagagacagatccattcgattagtgaacggatcggcactgcgtg
cgccaattctgcagacaaatggcagtattcatccacaattttaaaaga
aaaggggggattggggggtacagtgcaggggaaagaatagtagacata
atagcaacagacatacaaactaaagaattacaaaaacaaattacaaaa
attcaaaattttcgggtttattacagggacagcagagatccagtttgg
ttaattaatggggggacgttaacggggcggaacggtaccgagggccta
tttcccatgattccttcatatttgcatatacgatacaaggctgttaga
gagataattagaattaatttgactgtaaacacaaagatattagtacaa
aatacgtgacgtagaaagtaataatttcttgggtagtttgcagtttta
aaattatgttttaaaatggactatcatatgcttaccgtaacttgaaag
tatttcgatttcttggctttatatatcttgtggaaaggacgaaacacc
ggggcgcggacatggaggacggttttagagctagaaatagcaagttaa
aataaggctagtccgttatcaacttgaaaaagtggcaccgagtcggtg
cttttttgaattcgctagctaggtcttgaaaggagtgggaattggctc
cggtgcccgtcagtgggcagagcgcacatcgcccacagtccccgagaa
gttggggggaggggtcggcaattgatccggtgcctagagaaggtggcg
cggggtaaactgggaaagtgatgtcgtgtactggctccgcctttttcc
cgaggggggggagaaccgtatataagtgcagtagtcgccgtgaacgtt
ctttttcgcaacgggtttgccgccagaacacaggaccggtgccaccat
ggactataaggaccacgacggagactacaaggatcatgatattgatta
caaagacgatgacgataagatggccccaaagaagaagcggaaggtcgg
tatccacggagtcccagcagccgacaagaagtacagcatcggcctggc
catcggcaccaactctgtgggctgggccgtgatcaccgacgagtacaa
ggtgcccagcaagaaattcaaggtgctgggcaacaccgaccggcacag
catcaagaagaacctgatcggagccctgctgttcgacagcggcgaaac
agccgaggccacccggctgaagagaaccgccagaagaagatacaccag
acggaagaaccggatctgctatctgcaagagatcttcagcaacgagat
ggccaaggtggacgacagcttcttccacagactggaagagtccttcct
ggtggaagaggataagaagcacgagcggcaccccatcttcggcaacat
cgtggacgaggtggcctaccacgagaagtaccccaccatctaccacct
gagaaagaaactggtggacagcaccgacaaggccgacctgcggctgat
ctatctggccctggcccacatgatcaagttccggggccacttcctgat
cgagggcgacctgaaccccgacaacagcgacgtggacaagctgttcat
ccagctggtgcagacctacaaccagctgttcgaggaaaaccccatcaa
cgccagcggcgtggacgccaaggccatcctgtctgccagactgagcaa
gagcagacggctggaaaatctgatcgcccagctgcccggcgagaagaa
gaatggcctgttcggcaacctgattgccctgagcctgggcctgacccc
caacttcaagagcaacttcgacctggccgaggatgccaaactgcagct
gagcaaggacacctacgacgacgacctggacaacctgctggcccagat
cggcgaccagtacgccgacctgtttctggccgccaagaacctgtccga
cgccatcctgctgagcgacatcctgagagtgaacaccgagatcaccaa
ggcccccctgagcgcctctatgatcaagagatacgacgagcaccacca
ggacctgaccctgctgaaagctctcgtgcggcagcagctgcctgagaa
gtacaaagagattttcttcgaccagagcaagaacggctacgccggcta
cattgacggcggagccagccaggaagagttctacaagttcatcaagcc
catcctggaaaagatggacggcaccgaggaactgctcgtgaagctgaa
cagagaggacctgctgcggaagcagcggaccttcgacaacggcagcat
cccccaccagatccacctgggagagctgcacgccattctgcggcggca
ggaagatttttacccattcctgaaggacaaccgggaaaagatcgagaa
gatcctgaccttccgcatcccctactacgtgggccctctggccagggg
aaacagcagattcgcctggatgaccagaaagagcgaggaaaccatcac
cccctggaacttcgaggaagtggtggacaagggcgcttccgcccagag
cttcatcgagcggatgaccaacttcgataagaacctgcccaacgagaa
ggtgctgcccaagcacagcctgctgtacgagtacttcaccgtgtataa
cgagctgaccaaagtgaaatacgtgaccgagggaatgagaaagcccgc
cttcctgagcggcgagcagaaaaaggccatcgtggacctgctgttcaa
gaccaaccggaaagtgaccgtgaagcagctgaaagaggactacttcaa
gaaaatcgagtgcttcgactccgtggaaatctccggcgtggaagatcg
gttcaacgcctccctgggcacataccacgatctgctgaaaattatcaa
ggacaaggacttcctggacaatgaggaaaacgaggacattctggaaga
tatcgtgctgaccctgacactgtttgaggacagagagatgatcgagga
acggctgaaaacctatgcccacctgttcgacgacaaagtgatgaagca
gctgaagcggcggagatacaccggctggggcaggctgagccggaagct
gatcaacggcatccgggacaagcagtccggcaagacaatcctggattt
cctgaagtccgacggcttcgccaacagaaacttcatgcagctgatcca
cgacgacagcctgacctttaaagaggacatccagaaagcccaggtgtc
cggccagggcgatagcctgcacgagcacattgccaatctggccggcag
ccccgccattaagaagggcatcctgcagacagtgaaggtggtggacga
gctcgtgaaagtgatgggccggcacaagcccgagaacatcgtgatcga
aatggccagagagaaccagaccacccagaagggacagaagaacagccg
cgagagaatgaagcggatcgaagagggcatcaaagagctgggcagcca
gatcctgaaagaacaccccgtggaaaacacccagctgcagaacgagaa
gctgtacctgtactacctgcagaatggggggatatgtacgtggaccag
gaactggacatcaaccggctgtccgactacgatgtggacgctatcgtg
cctcagagctttctgaaggacgactccatcgacaacaaggtgctgacc
agaagcgacaagaaccggggcaagagcgacaacgtgccctccgaagag
gtcgtgaagaagatgaagaactactggcggcagctgctgaacgccaag
ctgattacccagagaaagttcgacaatctgaccaaggccgagagaggc
ggcctgagcgaactggataaggccggcttcatcaagagacagctggtg
gaaacccggcagatcacaaagcacgtggcacagatactagattcccga
atgaatacgaaatacgacgagaacgataagctgattcgggaagtcaaa
gtaatcactttaaagtcaaaattggtgtcggacttcagaaaggatttt
caattctataaagttagggagataaataactaccaccatgcgcacgac
gcttatcttaatgccgtcgtagggaccgcactcattaagaaatacccg
aagctagaaagtgagtttgtgtatggtgattacaaagtttatgacgtc
cgtaagatgatcgcgaaaagcgaacaggagataggcaaggctacagcc
aaatacttcttttattctaacattatgaatttctttaagacggaaatc
actctggcaaacggagagatacgcaaacgacctttaattgaaaccaat
ggggagacaggtgaaatcgtatgggataagggccgggacttcgcgacg
gtgagaaaagttttgtccatgccccaagtcaacatagtaaagaaaact
gaggtgcagaccggagggttttcaaaggaatcgattcttccaaaaagg
aatagtgataagctcatcgctcgtaaaaaggactgggacccgaaaaag
tacggtggcttcgtgagccctacagttgcctattctgtcctagtagtg
gcaaaagttgagaagggaaaatccaagaaactgaagtcagtcaaagaa
ttattggggataacgattatggagcgctcgtcttttgaaaagaacccc
atcgacttccttgaggcgaaaggttacaaggaagtaaaaaaggatctc
ataattaaactaccaaagtatagtctgtttgagttagaaaatggccga
aaacggatgttggctagcgccagagagcttcaaaaggggaacgaactc
gcactaccgtctaaatacgtgaatttcctgtatttagcgtcccattac
gagaagttgaaaggttcacctgaagataacgaacagaagcaacttttt
gttgagcagcacaaacattatctcgacgaaatcatagagcaaatttcg
gaattcagtaagagagtcatcctagctgatgccaatctggacaaagta
ttaagcgcatacaacaagcacagggataaacccatacgtgagcaggcg
gaaaatattatccatttgtttactcttaccaacctcggcgctccagcc
gcattcaagtattttgacacaacgatagatcgcaaagagtacagatct
accaaggaggtgctagacgcgacactgattcaccaatccatcacggga
ttatatgaaactcggatagatttgtcacagcttgggggtgacggatcc
cccaagaagaagaggaaagtcctcgagggcggaggcgggagcggatcc
ccctcccggctccagatgttcttcgctaataaccacgaccaggaattt
gaccctccaaaggtttacccacctgtcccagctgagaagaggaagccc
atccgggtgctgtctctctttgatggaatcgctacagggctcctggtg
ctgaaggacttgggcattcaggtggaccgctacattgcctcggaggtg
tgtgaggactccatcacggtgggcatggtgcggcaccaggggaagatc
atgtacgtcggggacgtccgcagcgtcacacagaagcatatccaggag
tggggcccattcgatctggtgattgggggcagtccctgcaatgacctc
tccatcgtcaaccctgctcgcaagggcctctacgagggcactggccgg
ctcttctttgagttctaccgcctcctgcatgatgcgcggcccaaggag
ggagatgatcgccccttcttctggctctttgagaatgtggtggccatg
ggcgttagtgacaagagggacatctcgcgatttctcgagtccaaccct
gtgatgattgatgccaaagaagtgtcagctgcacacagggcccgctac
ttctggggtaaccttcccggtatgaacaggccgttggcatccactgtg
aatgataagctggagctgcaggagtgtctggagcatggcaggatagcc
aagttcagcaaagtgaggaccattactacgaggtcaaactccataaag
cagggcaaagaccagcattttcctgtgttcatgaatgagaaagaggac
atcttatggtgcactgaaatggaaagggtatttggtttcccagtccac
tatactgacgtgtccaacatgagccgcttggcgaggcagagactgctg
ggccggtcatggagcgtgccagtcatccgccacctcttcgctccgctg
aaggagtattttgcgtgtgtgtccggccggcccggatccggcgcaaca
aacttctctctgctgaaacaagccggagatgtcgaagagaatcctgga
ccgaccgagtacaagcccacggtgcgcctcgccacccgcgacgacgtc
cccagggccgtacgcaccctcgccgccgcgttcgccgactaccccgcc
acgcgccacaccgtcgatccggaccgccacatcgagcgggtcaccgag
ctgcaagaactcttcctcacgcgcgtcgggctcgacatcggcaaggtg
tgggtcgcggacgacggcgccgcggtggcggtctggaccacgccggag
agcgtcgaagcgggggcggtgttcgccgagatcggcccgcgcatggcc
gagttgagcggttcccggctggccgcgcagcaacagatggaaggcctc
ctggcgccgcaccggcccaaggagcccgcgtggttcctggccaccgtc
ggagtctcgcccgaccaccagggcaagggtctgggcagcgccgtcgtg
ctccccggagtggaggcggccgagcgcgccggggtgcccgccttcctg
gagacctccgcgccccgcaacctccccttctacgagcggctcggcttc
accgtcaccgccgacgtcgaggtgcccgaaggaccgcgcacctggtgc
atgacccgcaagcccggtgcctgaacgcgttaagtcgacaatcaacct
ctggattacaaaatttgtgaaagattgactggtattcttaactatgtt
gctccttttacgctatgtggatacgctgctttaatgcctttgtatcat
gctattgcttcccgtatggctttcattttctcctccttgtataaatcc
tggttgctgtctctttatgaggagttgtggcccgttgtcaggcaacgt
ggcgtggtgtgcactgtgtttgctgacgcaacccccactggttggggc
attgccaccacctgtcagctcctttccgggactttcgctttccccctc
cctattgccacggcggaactcatcgccgcctgccttgcccgctgctgg
acaggggctcggctgttgggcactgacaattccgtggtgttgtcgggg
aaatcatcgtcctttccttggctgctcgcctgtgttgccacctggatt
ctgcgcgggacgtccttctgctacgtcccttcggccctcaatccagcg
gaccttccttcccgcggcctgctgccggctctgcggcctcttccgcgt
cttcgccttcgccctcagacgagtcggatctccctttgggccgcctcc
ccgcgtcgactttaagaccaatgacttacaaggcagctgtagatctta
gccactttttaaaagaaaaggggggactggaagggctaattcactccc
aacgaagacaagatctgctttttgcttgtactgggtctctctggttag
accagatctgagcctgggagctctctggctaactagggaacccactgc
ttaagcctcaataaagcttgccttgagtgcttcaagtagtgtgtgccc
gtctgttgtgtgactctggtaactagagatccctcagacccttttagt
cagtgtggaaaatctctagcagggcccgtttaaacccgctgatcagcc
tcgactgtgccttctagttgccagccatctgttgtttgcccctccccc
gtgccttccttgaccctggaaggtgccactcccactgtcctttcctaa
taaaatgaggaaattgcatcgcattgtctgagtaggtgtcattctatt
ctggggggtggggggggcaggacagcaagggggaggattgggaagaca
atagcaggcatgctggggatgcggtgggctctatggcttctgaggcgg
aaagaaccagctggggctctagggggtatccccacgcgccctgtagcg
gcgcattaagcgcggcgggtgtggtggttacgcgcagcgtgaccgcta
cacttgccagcgccctagcgcccgctcctttcgctttcttcccttcct
ttctcgccacgttcgccggctttccccgtcaagctctaaatcgggggc
tccctttagggttccgatttagtgctttacggcacctcgaccccaaaa
aacttgattagggtgatggttcacgtagtgggccatcgccctgataga
cggtttttcgccctttgacgttggagtccacgttctttaatagtggac
tcttgttccaaactggaacaacactcaaccctatctcggtctattctt
ttgatttataagggattttgccgatttcggcctattggttaaaaaatg
agctgatttaacaaaaatttaacgcgaattaattctgtggaatgtgtg
tcagttagggtgtggaaagtccccaggctccccagcaggcagaagtat
gcaaagcatgcatctcaattagtcagcaaccaggtgtggaaagtcccc
aggctccccagcaggcagaagtatgcaaagcatgcatctcaattagtc
agcaaccatagtcccgcccctaactccgcccatcccgcccctaactcc
gcccagttccgcccattctccgccccatggctgactaattttttttat
ttatgcagaggccgaggccgcctctgcctctgagctattccagaagta
gtgaggaggcttttttggaggcctaggcttttgcaaaaagctccctac
cgtcgacctctagctagagcttggcgtaatcatggtcatagctgtttc
ctgtgtgaaattgttatccgctcacaattccacacaacatacgagccg
gaagcataaagtgtaaagcctggggtgcctaatgagtgagctaactca
cattaattgcgttgcgctcactgcccgctttccagtcgggaaacctgt
cgtgccagctgcattaatgaatcggccaacgcgcggggagaggcggtt
tgcgtattgggcgctcttccgcttcctcgctcactgactcgctgcgct
cggtcgttcggctgcggcgagcggtatcagctcactcaaaggcggtaa
tacggttatccacagaatcaggggataacgcaggaaagaacatgtgag
caaaaggccagcaaaaggccaggaaccgtaaaaaggccgcgttgctgg
cgtttttccataggctccgcccccctgacgagcatcacaaaaatcgac
gctcaagtcagaggtggcgaaacccgacaggactataaagataccagg
cgtttccccctggaagctccctcgtgcgctctcctgttccgaccctgc
cgcttaccggatacctgtccgcctttctcccttcgggaagcgtggcgc
tttctcatagctcacgctgtaggtatctcagttcggtgtaggtcgttc
gctccaagctgggctgtgtgcacgaaccccccgttcagcccgaccgct
gcgccttatccggtaactatcgtcttgagtccaacccggtaagacacg
acttatcgccactggcagcagccactggtaacaggattagcagagcga
ggtatgtaggcggtgctacagagttcttgaagtggtggcctaactacg
gctacactagaagaacagtatttggtatctgcgctctgctgaagccag
ttaccttcggaaaaagagttggtagctcttgatccggcaaacaaacca
ccgctggtagcggtggtttttttgtttgcaagcagcagattacgcgca
gaaaaaaaggatctcaagaagatcctttgatcttttctacggggtctg
acgctcagtggaacgaaaactcacgttaagggattttggtcatgagat
tatcaaaaaggatcttcacctagatccttttaaattaaaaatgaagtt
ttaaatcaatctaaagtatatatgagtaaacttggtctgacagttacc
aatgcttaatcagtgaggcacctatctcagcgatctgtctatttcgtt
catccatagttgcctgactccccgtcgtgtagataactacgatacggg
agggcttaccatctggccccagtgctgcaatgataccgcgagacccac
gctcaccggctccagatttatcagcaataaaccagccagccggaaggg
ccgagcgcagaagtggtcctgcaactttatccgcctccatccagtcta
ttaattgttgccgggaagctagagtaagtagttcgccagttaatagtt
tgcgcaacgttgttgccattgctacaggcatcgtggtgtcacgctcgt
cgtttggtatggcttcattcagctccggttcccaacgatcaaggcgag
ttacatgatcccccatgttgtgcaaaaaagcggttagctccttcggtc
ctccgatcgttgtcagaagtaagttggccgcagtgttatcactcatgg
ttatggcagcactgcataattctcttactgtcatgccatccgtaagat
gcttttctgtgactggtgagtactcaaccaagtcattctgagaatagt
gtatgcggcgaccgagttgctcttgcccggcgtcaatacgggataata
ccgcgccacatagcagaactttaaaagtgctcatcattggaaaacgtt
cttcggggcgaaaactctcaaggatcttaccgctgttgagatccagtt
cgatgtaacccactcgtgcacccaactgatcttcagcatcttttactt
tcaccagcgtttctgggtgagcaaaaacaggaaggcaaaatgccgcaa
aaaagggaataagggcgacacggaaatgttgaatactcatactcttcc
tttttcaatattattgaagcatttatcagggttattgtctcatgagcg
gatacatatttgaatgtatttagaaaaataaacaaataggggttccgc
gcacatttccccgaaaagtgccacctgac.

In an aspect, a disclosed pBK1428 plasmid can comprise the sequence set forth below or a sequence having 70%, 75%, 80%, 85%, 90%, 95%, or more identity to the sequence set forth

(SEQ ID NO: 55)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggtggagtatttacggtaaactgcccacttggcagtacatcaag
tgtatcatatgccaagtacgccccctattgacgtcaatgacggtaaat
ggcccgcctggcattatgcccagtacatgaccttatgggactttccta
cttggcagtacatctacgtattagtcatcgctattaccatggtgatgc
ggttttggcagtacatcaatgggcgtggatagcggtttgactcacggg
gatttccaagtctccaccccattgacgtcaatgggagtttgttttggc
accaaaatcaacgggactttccaaaatgtcgtaacaactccgccccat
tgacgcaaatgggcggtaggcgtgtacgggggaggtctatataagcag
cgcgttttgcctgtactgggtctctctggttagaccagatctgagcct
gggagctctctggctaactagggaacccactgcttaagcctcaataaa
gcttgccttgagtgcttcaagtagtgtgtgcccgtctgttgtgtgact
ctggtaactagagatccctcagacccttttagtcagtgtggaaaatct
ctagcagtggcgcccgaacagggacttgaaagcgaaagggaaaccaga
ggagctctctcgacgcaggactcggcttgctgaagcgcgcacggcaag
aggcgaggggcggcgactggtgagtacgccaaaaattttgactagcgg
aggctagaaggagagagatgggtgcgagagcgtcagtattaagcgggg
gagaattagatcgcgatgggaaaaaattcggttaaggccagggggaaa
gaaaaaatataaattaaaacatatagtatgggcaagcagggagctaga
acgattcgcagttaatcctggcctgttagaaacatcagaaggctgtag
acaaatactgggacagctacaaccatcccttcagacaggatcagaaga
acttagatcattatataatacagtagcaaccctctattgtgtgcatca
aaggatagagataaaagacaccaaggaagctttagacaagatagagga
agagcaaaacaaaagtaagaccaccgcacagcaagcggccgctgatct
tcagacctggaggaggagatatgagggacaattggagaagtgaattat
ataaatataaagtagtaaaaattgaaccattaggagtagcacccacca
aggcaaagagaagagtggtgcagagagaaaaaagagcagtgggaatag
gagctttgttccttgggttcttgggagcagcaggaagcactatgggcg
cagcgtcaatgacgctgacggtacaggccagacaattattgtctggta
tagtgcagcagcagaacaatttgctgagggctattgaggcgcaacagc
atctgttgcaactcacagtctggggcatcaagcagctccaggcaagaa
tcctggctgtggaaagatacctaaaggatcaacagctcctggggattt
ggggttgctctggaaaactcatttgcaccactgctgtgccttggaatg
ctagttggagtaataaatctctggaacagatttggaatcacacgacct
ggatggagtgggacagagaaattaacaattacacaagcttaatacact
ccttaattgaagaatcgcaaaaccagcaagaaaagaatgaacaagaat
tattggaattagataaatgggcaagtttgtggaattggtttaacataa
caaattggctgtggtatataaaattattcataatgatagtaggaggct
tggtaggtttaagaatagtttttgctgtactttctatagtgaatagag
ttaggcagggatattcaccattatcgtttcagacccacctcccaaccc
cgaggggacccgacaggcccgaaggaatagaagaagaaggtggagaga
gagacagagacagatccattcgattagtgaacggatcggcactgcgtg
cgccaattctgcagacaaatggcagtattcatccacaattttaaaaga
aaaggggggattggggggtacagtgcaggggaaagaatagtagacata
atagcaacagacatacaaactaaagaattacaaaaacaaattacaaaa
attcaaaattttcgggtttattacagggacagcagagatccagtttgg
ttaattaatggggggacgttaacggggggaacggtaccgagggcctat
ttcccatgattccttcatatttgcatatacgatacaaggctgttagag
agataattagaattaatttgactgtaaacacaaagatattagtacaaa
atacgtgacgtagaaagtaataatttcttgggtagtttgcagttttaa
aattatgttttaaaatggactatcatatgcttaccgtaacttgaaagt
atttcgatttcttggctttatatatcttgtggaaaggacgaaacaccg
gcggtactgcaccaggcggcgttttagagctagaaatagcaagttaaa
ataaggctagtccgttatcaacttgaaaaagtggcaccgagtcggtgc
ttttttgaattcgctagctaggtcttgaaaggagtgggaattggctcc
ggtgcccgtcagtgggcagagcgcacatcgcccacagtccccgagaag
ttggggggaggggtcggcaattgatccggtgcctagagaaggtggcgc
ggggtaaactgggaaagtgatgtcgtgtactggctccgcctttttccc
gaggggggggagaaccgtatataagtgcagtagtcgccgtgaacgttc
tttttcgcaacgggtttgccgccagaacacaggaccggtgccaccatg
gactataaggaccacgacggagactacaaggatcatgatattgattac
aaagacgatgacgataagatggccccaaagaagaagcggaaggtcggt
atccacggagtcccagcagccgacaagaagtacagcatcggcctggcc
atcggcaccaactctgtgggctgggccgtgatcaccgacgagtacaag
gtgcccagcaagaaattcaaggtgctgggcaacaccgaccggcacagc
atcaagaagaacctgatcggagccctgctgttcgacagcggcgaaaca
gccgaggccacccggctgaagagaaccgccagaagaagatacaccaga
cggaagaaccggatctgctatctgcaagagatcttcagcaacgagatg
gccaaggtggacgacagcttcttccacagactggaagagtccttcctg
gtggaagaggataagaagcacgagcggcaccccatcttcggcaacatc
gtggacgaggtggcctaccacgagaagtaccccaccatctaccacctg
agaaagaaactggtggacagcaccgacaaggccgacctgcggctgatc
tatctggccctggcccacatgatcaagttccggggccacttcctgatc
gagggcgacctgaaccccgacaacagcgacgtggacaagctgttcatc
cagctggtgcagacctacaaccagctgttcgaggaaaaccccatcaac
gccagcggcgtggacgccaaggccatcctgtctgccagactgagcaag
agcagacggctggaaaatctgatcgcccagctgcccggcgagaagaag
aatggcctgttcggcaacctgattgccctgagcctgggcctgaccccc
aacttcaagagcaacttcgacctggccgaggatgccaaactgcagctg
agcaaggacacctacgacgacgacctggacaacctgctggcccagatc
ggcgaccagtacgccgacctgtttctggccgccaagaacctgtccgac
gccatcctgctgagcgacatcctgagagtgaacaccgagatcaccaag
gcccccctgagcgcctctatgatcaagagatacgacgagcaccaccag
gacctgaccctgctgaaagctctcgtgcggcagcagctgcctgagaag
tacaaagagattttcttcgaccagagcaagaacggctacgccggctac
attgacggcggagccagccaggaagagttctacaagttcatcaagccc
atcctggaaaagatggacggcaccgaggaactgctcgtgaagctgaac
agagaggacctgctgcggaagcagcggaccttcgacaacggcagcatc
ccccaccagatccacctgggagagctgcacgccattctgcggcggcag
gaagatttttacccattcctgaaggacaaccgggaaaagatcgagaag
atcctgaccttccgcatcccctactacgtgggccctctggccagggga
aacagcagattcgcctggatgaccagaaagagcgaggaaaccatcacc
ccctggaacttcgaggaagtggtggacaagggcgcttccgcccagagc
ttcatcgagcggatgaccaacttcgataagaacctgcccaacgagaag
gtgctgcccaagcacagcctgctgtacgagtacttcaccgtgtataac
gagctgaccaaagtgaaatacgtgaccgagggaatgagaaagcccgcc
ttcctgagcggcgagcagaaaaaggccatcgtggacctgctgttcaag
accaaccggaaagtgaccgtgaagcagctgaaagaggactacttcaag
aaaatcgagtgcttcgactccgtggaaatctccggcgtggaagatcgg
ttcaacgcctccctgggcacataccacgatctgctgaaaattatcaag
gacaaggacttcctggacaatgaggaaaacgaggacattctggaagat
atcgtgctgaccctgacactgtttgaggacagagagatgatcgaggaa
cggctgaaaacctatgcccacctgttcgacgacaaagtgatgaagcag
ctgaagcggcggagatacaccggctggggcaggctgagccggaagctg
atcaacggcatccgggacaagcagtccggcaagacaatcctggatttc
ctgaagtccgacggcttcgccaacagaaacttcatgcagctgatccac
gacgacagcctgacctttaaagaggacatccagaaagcccaggtgtcc
ggccagggcgatagcctgcacgagcacattgccaatctggccggcagc
cccgccattaagaagggcatcctgcagacagtgaaggtggtggacgag
ctcgtgaaagtgatgggccggcacaagcccgagaacatcgtgatcgaa
atggccagagagaaccagaccacccagaagggacagaagaacagccgc
gagagaatgaagcggatcgaagagggcatcaaagagctgggcagccag
atcctgaaagaacaccccgtggaaaacacccagctgcagaacgagaag
ctgtacctgtactacctgcagaatggggggatatgtacgtggaccagg
aactggacatcaaccggctgtccgactacgatgtggacgctatcgtgc
ctcagagctttctgaaggacgactccatcgacaacaaggtgctgacca
gaagcgacaagaaccggggcaagagcgacaacgtgccctccgaagagg
tcgtgaagaagatgaagaactactggcggcagctgctgaacgccaagc
tgattacccagagaaagttcgacaatctgaccaaggccgagagaggcg
gcctgagcgaactggataaggccggcttcatcaagagacagctggtgg
aaacccggcagatcacaaagcacgtggcacagatactagattcccgaa
tgaatacgaaatacgacgagaacgataagctgattcgggaagtcaaag
taatcactttaaagtcaaaattggtgtcggacttcagaaaggattttc
aattctataaagttagggagataaataactaccaccatgcgcacgacg
cttatcttaatgccgtcgtagggaccgcactcattaagaaatacccga
agctagaaagtgagtttgtgtatggtgattacaaagtttatgacgtcc
gtaagatgatcgcgaaaagcgaacaggagataggcaaggctacagcca
aatacttcttttattctaacattatgaatttctttaagacggaaatca
ctctggcaaacggagagatacgcaaacgacctttaattgaaaccaatg
gggagacaggtgaaatcgtatgggataagggccgggacttcgcgacgg
tgagaaaagttttgtccatgccccaagtcaacatagtaaagaaaactg
aggtgcagaccggagggttttcaaaggaatcgattcttccaaaaagga
atagtgataagctcatcgctcgtaaaaaggactgggacccgaaaaagt
acggtggcttcgtgagccctacagttgcctattctgtcctagtagtgg
caaaagttgagaagggaaaatccaagaaactgaagtcagtcaaagaat
tattggggataacgattatggagcgctcgtcttttgaaaagaacccca
tcgacttccttgaggcgaaaggttacaaggaagtaaaaaaggatctca
taattaaactaccaaagtatagtctgtttgagttagaaaatggccgaa
aacggatgttggctagcgccagagagcttcaaaaggggaacgaactcg
cactaccgtctaaatacgtgaatttcctgtatttagcgtcccattacg
agaagttgaaaggttcacctgaagataacgaacagaagcaactttttg
ttgagcagcacaaacattatctcgacgaaatcatagagcaaatttcgg
aattcagtaagagagtcatcctagctgatgccaatctggacaaagtat
taagcgcatacaacaagcacagggataaacccatacgtgagcaggcgg
aaaatattatccatttgtttactcttaccaacctcggcgctccagccg
cattcaagtattttgacacaacgatagatcgcaaagagtacagatcta
ccaaggaggtgctagacgcgacactgattcaccaatccatcacgggat
tatatgaaactcggatagatttgtcacagcttgggggtgacggatccc
ccaagaagaagaggaaagtcctcgagggcggaggcgggagcggatccc
cctcccggctccagatgttcttcgctaataaccacgaccaggaatttg
accctccaaaggtttacccacctgtcccagctgagaagaggaagccca
tccgggtgctgtctctctttgatggaatcgctacagggctcctggtgc
tgaaggacttgggcattcaggtggaccgctacattgcctcggaggtgt
gtgaggactccatcacggtgggcatggtgcggcaccaggggaagatca
tgtacgtcggggacgtccgcagcgtcacacagaagcatatccaggagt
ggggcccattcgatctggtgattgggggcagtccctgcaatgacctct
ccatcgtcaaccctgctcgcaagggcctctacgagggcactggccggc
tcttctttgagttctaccgcctcctgcatgatgcgcggcccaaggagg
gagatgatcgccccttcttctggctctttgcgaatgtggtggccatgg
gcgttagtgacaagagggacatctcgcgatttctcgagtccaaccctg
tgatgattgatgccaaagaagtgtcagctgcacacagggcccgctact
tctggggtaaccttcccggtatgaacaggccgttggcatccactgtga
atgataagctggagctgcaggagtgtctggagcatggcaggatagcca
agttcagcaaagtgaggaccattactacgaggtcaaactccataaagc
agggcaaagaccagcattttcctgtgttcatgaatgagaaagaggaca
tcttatggtgcactgaaatggaaagggtatttggtttcccagtccact
atactgacgtgtccaacatgagccgcttggcgaggcagagactgctgg
gccggtcatggagcgtgccagtcatccgccacctcttcgctccgctga
aggagtattttgcgtgtgtgtccggccggcccggatccggcgcaacaa
acttctctctgctgaaacaagccggagatgtcgaagagaatcctggac
cgaccgagtacaagcccacggtgcgcctcgccacccgcgacgacgtcc
ccagggccgtacgcaccctcgccgccgcgttcgccgactaccccgcca
cgcgccacaccgtcgatccggaccgccacatcgagcgggtcaccgagc
tgcaagaactcttcctcacgcgcgtcgggctcgacatcggcaaggtgt
gggtcgcggacgacggcgccgcggtggcggtctggaccacgccggaga
gcgtcgaagcgggggcggtgttcgccgagatcggcccgcgcatggccg
agttgagcggttcccggctggccgcgcagcaacagatggaaggcctcc
tggcgccgcaccggcccaaggagcccgcgtggttcctggccaccgtcg
gagtctcgcccgaccaccagggcaagggtctgggcagcgccgtcgtgc
tccccggagtggaggcggccgagcgcgccggggtgcccgccttcctgg
agacctccgcgccccgcaacctccccttctacgagcggctcggcttca
ccgtcaccgccgacgtcgaggtgcccgaaggaccgcgcacctggtgca
tgacccgcaagcccggtgcctgaacgcgttaagtcgacaatcaacctc
tggattacaaaatttgtgaaagattgactggtattcttaactatgttg
ctccttttacgctatgtggatacgctgctttaatgcctttgtatcatg
ctattgcttcccgtatggctttcattttctcctccttgtataaatcct
ggttgctgtctctttatgaggagttgtggcccgttgtcaggcaacgtg
gcgtggtgtgcactgtgtttgctgacgcaacccccactggttggggca
ttgccaccacctgtcagctcctttccgggactttcgctttccccctcc
ctattgccacggcggaactcatcgccgcctgccttgcccgctgctgga
caggggctcggctgttgggcactgacaattccgtggtgttgtcgggga
aatcatcgtcctttccttggctgctcgcctgtgttgccacctggattc
tgcgcgggacgtccttctgctacgtcccttcggccctcaatccagcgg
accttccttcccgcggcctgctgccggctctgcggcctcttccgcgtc
ttcgccttcgccctcagacgagtcggatctccctttgggccgcctccc
cgcgtcgactttaagaccaatgacttacaaggcagctgtagatcttag
ccactttttaaaagaaaaggggggactggaagggctaattcactccca
acgaagacaagatctgctttttgcttgtactgggtctctctggttaga
ccagatctgagcctgggagctctctggctaactagggaacccactgct
taagcctcaataaagcttgccttgagtgcttcaagtagtgtgtgcccg
tctgttgtgtgactctggtaactagagatccctcagacccttttagtc
agtgtggaaaatctctagcagggcccgtttaaacccgctgatcagcct
cgactgtgccttctagttgccagccatctgttgtttgcccctcccccg
tgccttccttgaccctggaaggtgccactcccactgtcctttcctaat
aaaatgaggaaattgcatcgcattgtctgagtaggtgtcattctattc
tggggggtggggggggcaggacagcaagggggaggattgggaagacaa
tagcaggcatgctggggatgcggtgggctctatggcttctgaggcgga
aagaaccagctggggctctagggggtatccccacgcgccctgtagcgg
cgcattaagcgcggcgggtgtggtggttacgcgcagcgtgaccgctac
acttgccagcgccctagcgcccgctcctttcgctttcttcccttcctt
tctcgccacgttcgccggctttccccgtcaagctctaaatcgggggct
ccctttagggttccgatttagtgctttacggcacctcgaccccaaaaa
acttgattagggtgatggttcacgtagtgggccatcgccctgatagac
ggtttttcgccctttgacgttggagtccacgttctttaatagtggact
cttgttccaaactggaacaacactcaaccctatctcggtctattcttt
tgatttataagggattttgccgatttcggcctattggttaaaaaatga
gctgatttaacaaaaatttaacgcgaattaattctgtggaatgtgtgt
cagttagggtgtggaaagtccccaggctccccagcaggcagaagtatg
caaagcatgcatctcaattagtcagcaaccaggtgtggaaagtcccca
ggctccccagcaggcagaagtatgcaaagcatgcatctcaattagtca
gcaaccatagtcccgcccctaactccgcccatcccgcccctaactccg
cccagttccgcccattctccgccccatggctgactaattttttttatt
tatgcagaggccgaggccgcctctgcctctgagctattccagaagtag
tgaggaggcttttttggaggcctaggcttttgcaaaaagctccctacc
gtcgacctctagctagagcttggcgtaatcatggtcatagctgtttcc
tgtgtgaaattgttatccgctcacaattccacacaacatacgagccgg
aagcataaagtgtaaagcctggggtgcctaatgagtgagctaactcac
attaattgcgttgcgctcactgcccgctttccagtcgggaaacctgtc
gtgccagctgcattaatgaatcggccaacgcgcggggagaggcggttt
gcgtattgggcgctcttccgcttcctcgctcactgactcgctgcgctc
ggtcgttcggctgcggcgagcggtatcagctcactcaaaggcggtaat
acggttatccacagaatcaggggataacgcaggaaagaacatgtgagc
aaaaggccagcaaaaggccaggaaccgtaaaaaggccgcgttgctggc
gtttttccataggctccgcccccctgacgagcatcacaaaaatcgacg
ctcaagtcagaggtggcgaaacccgacaggactataaagataccaggc
gtttccccctggaagctccctcgtgcgctctcctgttccgaccctgcc
gcttaccggatacctgtccgcctttctcccttcgggaagcgtggcgct
ttctcatagctcacgctgtaggtatctcagttcggtgtaggtcgttcg
ctccaagctgggctgtgtgcacgaaccccccgttcagcccgaccgctg
cgccttatccggtaactatcgtcttgagtccaacccggtaagacacga
cttatcgccactggcagcagccactggtaacaggattagcagagcgag
gtatgtaggcggtgctacagagttcttgaagtggtggcctaactacgg
ctacactagaagaacagtatttggtatctgcgctctgctgaagccagt
taccttcggaaaaagagttggtagctcttgatccggcaaacaaaccac
cgctggtagcggtggtttttttgtttgcaagcagcagattacgcgcag
aaaaaaaggatctcaagaagatcctttgatcttttctacggggtctga
cgctcagtggaacgaaaactcacgttaagggattttggtcatgagatt
atcaaaaaggatcttcacctagatccttttaaattaaaaatgaagttt
taaatcaatctaaagtatatatgagtaaacttggtctgacagttacca
atgcttaatcagtgaggcacctatctcagcgatctgtctatttcgttc
atccatagttgcctgactccccgtcgtgtagataactacgatacggga
gggcttaccatctggccccagtgctgcaatgataccgcgagacccacg
ctcaccggctccagatttatcagcaataaaccagccagccggaagggc
cgagcgcagaagtggtcctgcaactttatccgcctccatccagtctat
taattgttgccgggaagctagagtaagtagttcgccagttaatagttt
gcgcaacgttgttgccattgctacaggcatcgtggtgtcacgctcgtc
gtttggtatggcttcattcagctccggttcccaacgatcaaggcgagt
tacatgatcccccatgttgtgcaaaaaagcggttagctccttcggtcc
tccgatcgttgtcagaagtaagttggccgcagtgttatcactcatggt
tatggcagcactgcataattctcttactgtcatgccatccgtaagatg
cttttctgtgactggtgagtactcaaccaagtcattctgagaatagtg
tatgcggcgaccgagttgctcttgcccggcgtcaatacgggataatac
cgcgccacatagcagaactttaaaagtgctcatcattggaaaacgttc
ttcggggcgaaaactctcaaggatcttaccgctgttgagatccagttc
gatgtaacccactcgtgcacccaactgatcttcagcatcttttacttt
caccagcgtttctgggtgagcaaaaacaggaaggcaaaatgccgcaaa
aaagggaataagggcgacacggaaatgttgaatactcatactcttcct
ttttcaatattattgaagcatttatcagggttattgtctcatgagcgg
atacatatttgaatgtatttagaaaaataaacaaataggggttccgcg
cacatttccccgaaaagtgccacct.

In an aspect, a disclosed pBK1428 plasmid can comprise the sequence set forth below or a sequence having 70%, 75%, 80%, 85%, 90%, 95%, or more identity to the sequence set forth

(SEQ ID NO: 56)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggtggagtatttacggtaaactgcccacttggcagtacatcaag
tgtatcatatgccaagtacgccccctattgacgtcaatgacggtaaat
ggcccgcctggcattatgcccagtacatgaccttatgggactttccta
cttggcagtacatctacgtattagtcatcgctattaccatggtgatgc
ggttttggcagtacatcaatgggcgtggatagcggtttgactcacggg
gatttccaagtctccaccccattgacgtcaatgggagtttgttttggc
accaaaatcaacgggactttccaaaatgtcgtaacaactccgccccat
tgacgcaaatgggcggtaggcgtgtacggtgggaggtctatataagca
gcgcgttttgcctgtactgggtctctctggttagaccagatctgagcc
tgggagctctctggctaactagggaacccactgcttaagcctcaataa
agcttgccttgagtgcttcaagtagtgtgtgcccgtctgttgtgtgac
tctggtaactagagatccctcagacccttttagtcagtgtggaaaatc
tctagcagtggcgcccgaacagggacttgaaagcgaaagggaaaccag
aggagctctctcgacgcaggactcggcttgctgaagcgcgcacggcaa
gaggcgaggggcggcgactggtgagtacgccaaaaattttgactagcg
gaggctagaaggagagagatgggtgcgagagcgtcagtattaagcggg
ggagaattagatcgcgatgggaaaaaattcggttaaggccagggggaa
agaaaaaatataaattaaaacatatagtatgggcaagcagggagctag
aacgattcgcagttaatcctggcctgttagaaacatcagaaggctgta
gacaaatactgggacagctacaaccatcccttcagacaggatcagaag
aacttagatcattatataatacagtagcaaccctctattgtgtgcatc
aaaggatagagataaaagacaccaaggaagctttagacaagatagagg
aagagcaaaacaaaagtaagaccaccgcacagcaagcggccgctgatc
ttcagacctggaggaggagatatgagggacaattggagaagtgaatta
tataaatataaagtagtaaaaattgaaccattaggagtagcacccacc
aaggcaaagagaagagtggtgcagagagaaaaaagagcagtgggaata
ggagctttgttccttgggttcttgggagcagcaggaagcactatgggc
gcagcgtcaatgacgctgacggtacaggccagacaattattgtctggt
atagtgcagcagcagaacaatttgctgagggctattgaggcgcaacag
catctgttgcaactcacagtctggggcatcaagcagctccaggcaaga
atcctggctgtggaaagatacctaaaggatcaacagctcctggggatt
tggggttgctctggaaaactcatttgcaccactgctgtgccttggaat
gctagttggagtaataaatctctggaacagatttggaatcacacgacc
tggatggagtgggacagagaaattaacaattacacaagcttaatacac
tccttaattgaagaatcgcaaaaccagcaagaaaagaatgaacaagaa
ttattggaattagataaatgggcaagtttgtggaattggtttaacata
acaaattggctgtggtatataaaattattcataatgatagtaggaggc
ttggtaggtttaagaatagtttttgctgtactttctatagtgaataga
gttaggcagggatattcaccattatcgtttcagacccacctcccaacc
ccgaggggacccgacaggcccgaaggaatagaagaagaaggtggagag
agagacagagacagatccattcgattagtgaacggatcggcactgcgt
gcgccaattctgcagacaaatggcagtattcatccacaattttaaaag
aaaaggggggattggggggtacagtgcaggggaaagaatagtagacat
aatagcaacagacatacaaactaaagaattacaaaaacaaattacaaa
aattcaaaattttcgggtttattacagggacagcagagatccagtttg
gttaattaatggggggacgttaacggggggaacggtaccgagggccta
tttcccatgattccttcatatttgcatatacgatacaaggctgttaga
gagataattagaattaatttgactgtaaacacaaagatattagtacaa
aatacgtgacgtagaaagtaataatttcttgggtagtttgcagtttta
aaattatgttttaaaatggactatcatatgcttaccgtaacttgaaag
tatttcgatttcttggctttatatatcttgtggaaaggacgaaacacc
ggggcgcggacatggaggacggttttagagctagaaatagcaagttaa
aataaggctagtccgttatcaacttgaaaaagtggcaccgagtcggtg
cttttttgaattcgctagctaggtcttgaaaggagtgggaattggctc
cggtgcccgtcagtgggcagagcgcacatcgcccacagtccccgagaa
gttggggggaggggtcggcaattgatccggtgcctagagaaggtggcg
cggggtaaactgggaaagtgatgtcgtgtactggctccgcctttttcc
cgaggggggggagaaccgtatataagtgcagtagtcgccgtgaacgtt
ctttttcgcaacgggtttgccgccagaacacaggaccggtgccaccat
ggactataaggaccacgacggagactacaaggatcatgatattgatta
caaagacgatgacgataagatggccccaaagaagaagcggaaggtcgg
tatccacggagtcccagcagccgacaagaagtacagcatcggcctggc
catcggcaccaactctgtgggctgggccgtgatcaccgacgagtacaa
ggtgcccagcaagaaattcaaggtgctgggcaacaccgaccggcacag
catcaagaagaacctgatcggagccctgctgttcgacagcggcgaaac
agccgaggccacccggctgaagagaaccgccagaagaagatacaccag
acggaagaaccggatctgctatctgcaagagatcttcagcaacgagat
ggccaaggtggacgacagcttcttccacagactggaagagtccttcct
ggtggaagaggataagaagcacgagcggcaccccatcttcggcaacat
cgtggacgaggtggcctaccacgagaagtaccccaccatctaccacct
gagaaagaaactggtggacagcaccgacaaggccgacctgcggctgat
ctatctggccctggcccacatgatcaagttccggggccacttcctgat
cgagggcgacctgaaccccgacaacagcgacgtggacaagctgttcat
ccagctggtgcagacctacaaccagctgttcgaggaaaaccccatcaa
cgccagcggcgtggacgccaaggccatcctgtctgccagactgagcaa
gagcagacggctggaaaatctgatcgcccagctgcccggcgagaagaa
gaatggcctgttcggcaacctgattgccctgagcctgggcctgacccc
caacttcaagagcaacttcgacctggccgaggatgccaaactgcagct
gagcaaggacacctacgacgacgacctggacaacctgctggcccagat
cggcgaccagtacgccgacctgtttctggccgccaagaacctgtccga
cgccatcctgctgagcgacatcctgagagtgaacaccgagatcaccaa
ggcccccctgagcgcctctatgatcaagagatacgacgagcaccacca
ggacctgaccctgctgaaagctctcgtgcggcagcagctgcctgagaa
gtacaaagagattttcttcgaccagagcaagaacggctacgccggcta
cattgacggcggagccagccaggaagagttctacaagttcatcaagcc
catcctggaaaagatggacggcaccgaggaactgctcgtgaagctgaa
cagagaggacctgctgcggaagcagcggaccttcgacaacggcagcat
cccccaccagatccacctgggagagctgcacgccattctgcggcggca
ggaagatttttacccattcctgaaggacaaccgggaaaagatcgagaa
gatcctgaccttccgcatcccctactacgtgggccctctggccagggg
aaacagcagattcgcctggatgaccagaaagagcgaggaaaccatcac
cccctggaacttcgaggaagtggtggacaagggcgcttccgcccagag
cttcatcgagcggatgaccaacttcgataagaacctgcccaacgagaa
ggtgctgcccaagcacagcctgctgtacgagtacttcaccgtgtataa
cgagctgaccaaagtgaaatacgtgaccgagggaatgagaaagcccgc
cttcctgagcggcgagcagaaaaaggccatcgtggacctgctgttcaa
gaccaaccggaaagtgaccgtgaagcagctgaaagaggactacttcaa
gaaaatcgagtgcttcgactccgtggaaatctccggcgtggaagatcg
gttcaacgcctccctgggcacataccacgatctgctgaaaattatcaa
ggacaaggacttcctggacaatgaggaaaacgaggacattctggaaga
tatcgtgctgaccctgacactgtttgaggacagagagatgatcgagga
acggctgaaaacctatgcccacctgttcgacgacaaagtgatgaagca
gctgaagcggcggagatacaccggctggggcaggctgagccggaagct
gatcaacggcatccgggacaagcagtccggcaagacaatcctggattt
cctgaagtccgacggcttcgccaacagaaacttcatgcagctgatcca
cgacgacagcctgacctttaaagaggacatccagaaagcccaggtgtc
cggccagggcgatagcctgcacgagcacattgccaatctggccggcag
ccccgccattaagaagggcatcctgcagacagtgaaggtggtggacga
gctcgtgaaagtgatgggccggcacaagcccgagaacatcgtgatcga
aatggccagagagaaccagaccacccagaagggacagaagaacagccg
cgagagaatgaagcggatcgaagagggcatcaaagagctgggcagcca
gatcctgaaagaacaccccgtggaaaacacccagctgcagaacgagaa
gctgtacctgtactacctgcagaatggggggatatgtacgtggaccag
gaactggacatcaaccggctgtccgactacgatgtggacgctatcgtg
cctcagagctttctgaaggacgactccatcgacaacaaggtgctgacc
agaagcgacaagaaccggggcaagagcgacaacgtgccctccgaagag
gtcgtgaagaagatgaagaactactggcggcagctgctgaacgccaag
ctgattacccagagaaagttcgacaatctgaccaaggccgagagaggc
ggcctgagcgaactggataaggccggcttcatcaagagacagctggtg
gaaacccggcagatcacaaagcacgtggcacagatactagattcccga
atgaatacgaaatacgacgagaacgataagctgattcgggaagtcaaa
gtaatcactttaaagtcaaaattggtgtcggacttcagaaaggatttt
caattctataaagttagggagataaataactaccaccatgcgcacgac
gcttatcttaatgccgtcgtagggaccgcactcattaagaaatacccg
aagctagaaagtgagtttgtgtatggtgattacaaagtttatgacgtc
cgtaagatgatcgcgaaaagcgaacaggagataggcaaggctacagcc
aaatacttcttttattctaacattatgaatttctttaagacggaaatc
actctggcaaacggagagatacgcaaacgacctttaattgaaaccaat
ggggagacaggtgaaatcgtatgggataagggccgggacttcgcgacg
gtgagaaaagttttgtccatgccccaagtcaacatagtaaagaaaact
gaggtgcagaccggagggttttcaaaggaatcgattcttccaaaaagg
aatagtgataagctcatcgctcgtaaaaaggactgggacccgaaaaag
tacggtggcttcgtgagccctacagttgcctattctgtcctagtagtg
gcaaaagttgagaagggaaaatccaagaaactgaagtcagtcaaagaa
ttattggggataacgattatggagcgctcgtcttttgaaaagaacccc
atcgacttccttgaggcgaaaggttacaaggaagtaaaaaaggatctc
ataattaaactaccaaagtatagtctgtttgagttagaaaatggccga
aaacggatgttggctagcgccagagagcttcaaaaggggaacgaactc
gcactaccgtctaaatacgtgaatttcctgtatttagcgtcccattac
gagaagttgaaaggttcacctgaagataacgaacagaagcaacttttt
gttgagcagcacaaacattatctcgacgaaatcatagagcaaatttcg
gaattcagtaagagagtcatcctagctgatgccaatctggacaaagta
ttaagcgcatacaacaagcacagggataaacccatacgtgagcaggcg
gaaaatattatccatttgtttactcttaccaacctcggcgctccagcc
gcattcaagtattttgacacaacgatagatcgcaaagagtacagatct
accaaggaggtgctagacgcgacactgattcaccaatccatcacggga
ttatatgaaactcggatagatttgtcacagcttgggggtgacggatcc
cccaagaagaagaggaaagtcctcgagggcggaggcgggagcggatcc
ccctcccggctccagatgttcttcgctaataaccacgaccaggaattt
gaccctccaaaggtttacccacctgtcccagctgagaagaggaagccc
atccgggtgctgtctctctttgatggaatcgctacagggctcctggtg
ctgaaggacttgggcattcaggtggaccgctacattgcctcggaggtg
tgtgaggactccatcacggtgggcatggtgcggcaccaggggaagatc
atgtacgtcggggacgtccgcagcgtcacacagaagcatatccaggag
tggggcccattcgatctggtgattgggggcagtccctgcaatgacctc
tccatcgtcaaccctgctcgcaagggcctctacgagggcactggccgg
ctcttctttgagttctaccgcctcctgcatgatgcgcggcccaaggag
ggagatgatcgccccttcttctggctctttgcgaatgtggtggccatg
ggcgttagtgacaagagggacatctcgcgatttctcgagtccaaccct
gtgatgattgatgccaaagaagtgtcagctgcacacagggcccgctac
ttctggggtaaccttcccggtatgaacaggccgttggcatccactgtg
aatgataagctggagctgcaggagtgtctggagcatggcaggatagcc
aagttcagcaaagtgaggaccattactacgaggtcaaactccataaag
cagggcaaagaccagcattttcctgtgttcatgaatgagaaagaggac
atcttatggtgcactgaaatggaaagggtatttggtttcccagtccac
tatactgacgtgtccaacatgagccgcttggcgaggcagagactgctg
ggccggtcatggagcgtgccagtcatccgccacctcttcgctccgctg
aaggagtattttgcgtgtgtgtccggccggcccggatccggcgcaaca
aacttctctctgctgaaacaagccggagatgtcgaagagaatcctgga
ccgaccgagtacaagcccacggtgcgcctcgccacccgcgacgacgtc
cccagggccgtacgcaccctcgccgccgcgttcgccgactaccccgcc
acgcgccacaccgtcgatccggaccgccacatcgagcgggtcaccgag
ctgcaagaactcttcctcacgcgcgtcgggctcgacatcggcaaggtg
tgggtcgcggacgacggcgccgcggtggcggtctggaccacgccggag
agcgtcgaagcgggggcggtgttcgccgagatcggcccgcgcatggcc
gagttgagcggttcccggctggccgcgcagcaacagatggaaggcctc
ctggcgccgcaccggcccaaggagcccgcgtggttcctggccaccgtc
ggagtctcgcccgaccaccagggcaagggtctgggcagcgccgtcgtg
ctccccggagtggaggcggccgagcgcgccggggtgcccgccttcctg
gagacctccgcgccccgcaacctccccttctacgagcggctcggcttc
accgtcaccgccgacgtcgaggtgcccgaaggaccgcgcacctggtgc
atgacccgcaagcccggtgcctgaacgcgttaagtcgacaatcaacct
ctggattacaaaatttgtgaaagattgactggtattcttaactatgtt
gctccttttacgctatgtggatacgctgctttaatgcctttgtatcat
gctattgcttcccgtatggctttcattttctcctccttgtataaatcc
tggttgctgtctctttatgaggagttgtggcccgttgtcaggcaacgt
ggcgtggtgtgcactgtgtttgctgacgcaacccccactggttggggc
attgccaccacctgtcagctcctttccgggactttcgctttccccctc
cctattgccacggcggaactcatcgccgcctgccttgcccgctgctgg
acaggggctcggctgttgggcactgacaattccgtggtgttgtcgggg
aaatcatcgtcctttccttggctgctcgcctgtgttgccacctggatt
ctgcgcgggacgtccttctgctacgtcccttcggccctcaatccagcg
gaccttccttcccgcggcctgctgccggctctgcggcctcttccgcgt
cttcgccttcgccctcagacgagtcggatctccctttgggccgcctcc
ccgcgtcgactttaagaccaatgacttacaaggcagctgtagatctta
gccactttttaaaagaaaaggggggactggaagggctaattcactccc
aacgaagacaagatctgctttttgcttgtactgggtctctctggttag
accagatctgagcctgggagctctctggctaactagggaacccactgc
ttaagcctcaataaagcttgccttgagtgcttcaagtagtgtgtgccc
gtctgttgtgtgactctggtaactagagatccctcagacccttttagt
cagtgtggaaaatctctagcagggcccgtttaaacccgctgatcagcc
tcgactgtgccttctagttgccagccatctgttgtttgcccctccccc
gtgccttccttgaccctggaaggtgccactcccactgtcctttcctaa
taaaatgaggaaattgcatcgcattgtctgagtaggtgtcattctatt
ctggggggtggggggggcaggacagcaagggggaggattgggaagaca
atagcaggcatgctggggatgcggtgggctctatggcttctgaggcgg
aaagaaccagctggggctctagggggtatccccacgcgccctgtagcg
gcgcattaagcgcggcgggtgtggtggttacgcgcagcgtgaccgcta
cacttgccagcgccctagcgcccgctcctttcgctttcttcccttcct
ttctcgccacgttcgccggctttccccgtcaagctctaaatcgggggc
tccctttagggttccgatttagtgctttacggcacctcgaccccaaaa
aacttgattagggtgatggttcacgtagtgggccatcgccctgataga
cggtttttcgccctttgacgttggagtccacgttctttaatagtggac
tcttgttccaaactggaacaacactcaaccctatctcggtctattctt
ttgatttataagggattttgccgatttcggcctattggttaaaaaatg
agctgatttaacaaaaatttaacgcgaattaattctgtggaatgtgtg
tcagttagggtgtggaaagtccccaggctccccagcaggcagaagtat
gcaaagcatgcatctcaattagtcagcaaccaggtgtggaaagtcccc
aggctccccagcaggcagaagtatgcaaagcatgcatctcaattagtc
agcaaccatagtcccgcccctaactccgcccatcccgcccctaactcc
gcccagttccgcccattctccgccccatggctgactaattttttttat
ttatgcagaggccgaggccgcctctgcctctgagctattccagaagta
gtgaggaggcttttttggaggcctaggcttttgcaaaaagctccctac
cgtcgacctctagctagagcttggcgtaatcatggtcatagctgtttc
ctgtgtgaaattgttatccgctcacaattccacacaacatacgagccg
gaagcataaagtgtaaagcctggggtgcctaatgagtgagctaactca
cattaattgcgttgcgctcactgcccgctttccagtcgggaaacctgt
cgtgccagctgcattaatgaatcggccaacgcgcggggagaggcggtt
tgcgtattgggcgctcttccgcttcctcgctcactgactcgctgcgct
cggtcgttcggctgcggcgagcggtatcagctcactcaaaggcggtaa
tacggttatccacagaatcaggggataacgcaggaaagaacatgtgag
caaaaggccagcaaaaggccaggaaccgtaaaaaggccgcgttgctgg
cgtttttccataggctccgcccccctgacgagcatcacaaaaatcgac
gctcaagtcagaggtggcgaaacccgacaggactataaagataccagg
cgtttccccctggaagctccctcgtgcgctctcctgttccgaccctgc
cgcttaccggatacctgtccgcctttctcccttcgggaagcgtggcgc
tttctcatagctcacgctgtaggtatctcagttcggtgtaggtcgttc
gctccaagctgggctgtgtgcacgaaccccccgttcagcccgaccgct
gcgccttatccggtaactatcgtcttgagtccaacccggtaagacacg
acttatcgccactggcagcagccactggtaacaggattagcagagcga
ggtatgtaggcggtgctacagagttcttgaagtggtggcctaactacg
gctacactagaagaacagtatttggtatctgcgctctgctgaagccag
ttaccttcggaaaaagagttggtagctcttgatccggcaaacaaacca
ccgctggtagcggtggtttttttgtttgcaagcagcagattacgcgca
gaaaaaaaggatctcaagaagatcctttgatcttttctacggggtctg
acgctcagtggaacgaaaactcacgttaagggattttggtcatgagat
tatcaaaaaggatcttcacctagatccttttaaattaaaaatgaagtt
ttaaatcaatctaaagtatatatgagtaaacttggtctgacagttacc
aatgcttaatcagtgaggcacctatctcagcgatctgtctatttcgtt
catccatagttgcctgactccccgtcgtgtagataactacgatacggg
agggcttaccatctggccccagtgctgcaatgataccgcgagacccac
gctcaccggctccagatttatcagcaataaaccagccagccggaaggg
ccgagcgcagaagtggtcctgcaactttatccgcctccatccagtcta
ttaattgttgccgggaagctagagtaagtagttcgccagttaatagtt
tgcgcaacgttgttgccattgctacaggcatcgtggtgtcacgctcgt
cgtttggtatggcttcattcagctccggttcccaacgatcaaggcgag
ttacatgatcccccatgttgtgcaaaaaagcggttagctccttcggtc
ctccgatcgttgtcagaagtaagttggccgcagtgttatcactcatgg
ttatggcagcactgcataattctcttactgtcatgccatccgtaagat
gcttttctgtgactggtgagtactcaaccaagtcattctgagaatagt
gtatgcggcgaccgagttgctcttgcccggcgtcaatacgggataata
ccgcgccacatagcagaactttaaaagtgctcatcattggaaaacgtt
cttcggggcgaaaactctcaaggatcttaccgctgttgagatccagtt
cgatgtaacccactcgtgcacccaactgatcttcagcatcttttactt
tcaccagcgtttctgggtgagcaaaaacaggaaggcaaaatgccgcaa
aaaagggaataagggcgacacggaaatgttgaatactcatactcttcc
tttttcaatattattgaagcatttatcagggttattgtctcatgagcg
gatacatatttgaatgtatttagaaaaataaacaaataggggttccgc
gcacatttccccgaaaagtgccacctgac.

In an aspect, a disclosed pBK1531 plasmid can comprise the sequence set forth below or a sequence having 70%, 75%, 80%, 85%, 90%, 95%, or more identity to the sequence set forth

(SEQ ID NO: 59)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggtggagtatttacggtaaactgcccacttggcagtacatcaag
tgtatcatatgccaagtacgccccctattgacgtcaatgacggtaaat
ggcccgcctggcattatgcccagtacatgaccttatgggactttccta
cttggcagtacatctacgtattagtcatcgctattaccatggtgatgc
ggttttggcagtacatcaatgggcgtggatagcggtttgactcacggg
gatttccaagtctccaccccattgacgtcaatgggagtttgttttggc
accaaaatcaacgggactttccaaaatgtcgtaacaactccgccccat
tgacgcaaatgggcggtaggcgtgtacggtgggaggtctatataagca
gcgcgttttgcctgtactgggtctctctggttagaccagatctgagcc
tgggagctctctggctaactagggaacccactgcttaagcctcaataa
agcttgccttgagtgcttcaagtagtgtgtgcccgtctgttgtgtgac
tctggtaactagagatccctcagacccttttagtcagtgtggaaaatc
tctagcagtggcgcccgaacagggacttgaaagcgaaagggaaaccag
aggagctctctcgacgcaggactcggcttgctgaagcgcgcacggcaa
gaggcgaggggcggcgactggtgagtacgccaaaaattttgactagcg
gaggctagaaggagagagatgggtgcgagagcgtcagtattaagcggg
ggagaattagatcgcgatgggaaaaaattcggttaaggccagggggaa
agaaaaaatataaattaaaacatatagtatgggcaagcagggagctag
aacgattcgcagttaatcctggcctgttagaaacatcagaaggctgta
gacaaatactgggacagctacaaccatcccttcagacaggatcagaag
aacttagatcattatataatacagtagcaaccctctattgtgtgcatc
aaaggatagagataaaagacaccaaggaagctttagacaagatagagg
aagagcaaaacaaaagtaagaccaccgcacagcaagcggccgctgatc
ttcagacctggaggaggagatatgagggacaattggagaagtgaatta
tataaatataaagtagtaaaaattgaaccattaggagtagcacccacc
aaggcaaagagaagagtggtgcagagagaaaaaagagcagtgggaata
ggagctttgttccttgggttcttgggagcagcaggaagcactatgggc
gcagcgtcaatgacgctgacggtacaggccagacaattattgtctggt
atagtgcagcagcagaacaatttgctgagggctattgaggcgcaacag
catctgttgcaactcacagtctggggcatcaagcagctccaggcaaga
atcctggctgtggaaagatacctaaaggatcaacagctcctggggatt
tggggttgctctggaaaactcatttgcaccactgctgtgccttggaat
gctagttggagtaataaatctctggaacagatttggaatcacacgacc
tggatggagtgggacagagaaattaacaattacacaagcttaatacac
tccttaattgaagaatcgcaaaaccagcaagaaaagaatgaacaagaa
ttattggaattagataaatgggcaagtttgtggaattggtttaacata
acaaattggctgtggtatataaaattattcataatgatagtaggaggc
ttggtaggtttaagaatagtttttgctgtactttctatagtgaataga
gttaggcagggatattcaccattatcgtttcagacccacctcccaacc
ccgaggggacccgacaggcccgaaggaatagaagaagaaggtggagag
agagacagagacagatccattcgattagtgaacggatcggcactgcgt
gcgccaattctgcagacaaatggcagtattcatccacaattttaaaag
aaaaggggggattggggggtacagtgcaggggaaagaatagtagacat
aatagcaacagacatacaaactaaagaattacaaaaacaaattacaaa
aattcaaaattttcgggtttattacagggacagcagagatccagtttg
gttaattaatggggggacgttaacggggcggaacggtaccgagggcct
atttcccatgattccttcatatttgcatatacgatacaaggctgttag
agagataattagaattaatttgactgtaaacacaaagatattagtaca
aaatacgtgacgtagaaagtaataatttcttgggtagtttgcagtttt
aaaattatgttttaaaatggactatcatatgcttaccgtaacttgaaa
gtatttcgatttcttggctttatatatcttgtggaaaggacgaaacac
cggcggtactgcaccaggcggcgttttagagctagaaatagcaagtta
aaataaggctagtccgttatcaacttgaaaaagtggcaccgagtcggt
gcttttttgaattcgctagctaggtcttgaaaggagtgggaattggct
ccggtgcccgtcagtgggcagagcgcacatcgcccacagtccccgaga
agttggggggaggggtcggcaattgatccggtgcctagagaaggtggc
gcggggtaaactgggaaagtgatgtcgtgtactggctccgcctttttc
ccgaggggggggagaaccgtatataagtgcagtagtcgccgtgaacgt
tctttttcgcaacgggtttgccgccagaacacaggaccggtgccacca
tggactataaggaccacgacggagactacaaggatcatgatattgatt
acaaagacgatgacgataagatggccccaaagaagaagcggaaggtcg
gtatccacggagtcccagcagccgacaagaagtacagcatcggcctgg
ccatcggcaccaactctgtgggctgggccgtgatcaccgacgagtaca
aggtgcccagcaagaaattcaaggtgctgggcaacaccgaccggcaca
gcatcaagaagaacctgatcggagccctgctgttcgacagcggcgaaa
cagccgaggccacccggctgaagagaaccgccagaagaagatacacca
gacggaagaaccggatctgctatctgcaagagatcttcagcaacgaga
tggccaaggtggacgacagcttcttccacagactggaagagtccttcc
tggtggaagaggataagaagcacgagcggcaccccatcttcggcaaca
tcgtggacgaggtggcctaccacgagaagtaccccaccatctaccacc
tgagaaagaaactggtggacagcaccgacaaggccgacctgcggctga
tctatctggccctggcccacatgatcaagttccggggccacttcctga
tcgagggcgacctgaaccccgacaacagcgacgtggacaagctgttca
tccagctggtgcagacctacaaccagctgttcgaggaaaaccccatca
acgccagcggcgtggacgccaaggccatcctgtctgccagactgagca
agagcagacggctggaaaatctgatcgcccagctgcccggcgagaaga
agaatggcctgttcggcaacctgattgccctgagcctgggcctgaccc
ccaacttcaagagcaacttcgacctggccgaggatgccaaactgcagc
tgagcaaggacacctacgacgacgacctggacaacctgctggcccaga
tcggcgaccagtacgccgacctgtttctggccgccaagaacctgtccg
acgccatcctgctgagcgacatcctgagagtgaacaccgagatcacca
aggcccccctgagcgcctctatgatcaagagatacgacgagcaccacc
aggacctgaccctgctgaaagctctcgtgcggcagcagctgcctgaga
agtacaaagagattttcttcgaccagagcaagaacggctacgccggct
acattgacggcggagccagccaggaagagttctacaagttcatcaagc
ccatcctggaaaagatggacggcaccgaggaactgctcgtgaagctga
acagagaggacctgctgcggaagcagcggaccttcgacaacggcagca
tcccccaccagatccacctgggagagctgcacgccattctgcggcggc
aggaagatttttacccattcctgaaggacaaccgggaaaagatcgaga
agatcctgaccttccgcatcccctactacgtgggccctctggccaggg
gaaacagcagattcgcctggatgaccagaaagagcgaggaaaccatca
ccccctggaacttcgaggaagtggtggacaagggcgcttccgcccaga
gcttcatcgagcggatgaccaacttcgataagaacctgcccaacgaga
aggtgctgcccaagcacagcctgctgtacgagtacttcaccgtgtata
acgagctgaccaaagtgaaatacgtgaccgagggaatgagaaagcccg
ccttcctgagcggcgagcagaaaaaggccatcgtggacctgctgttca
agaccaaccggaaagtgaccgtgaagcagctgaaagaggactacttca
agaaaatcgagtgcttcgactccgtggaaatctccggcgtggaagatc
ggttcaacgcctccctgggcacataccacgatctgctgaaaattatca
aggacaaggacttcctggacaatgaggaaaacgaggacattctggaag
atatcgtgctgaccctgacactgtttgaggacagagagatgatcgagg
aacggctgaaaacctatgcccacctgttcgacgacaaagtgatgaagc
agctgaagcggcggagatacaccggctggggcaggctgagccggaagc
tgatcaacggcatccgggacaagcagtccggcaagacaatcctggatt
tcctgaagtccgacggcttcgccaacagaaacttcatgcagctgatcc
acgacgacagcctgacctttaaagaggacatccagaaagcccaggtgt
ccggccagggcgatagcctgcacgagcacattgccaatctggccggca
gccccgccattaagaagggcatcctgcagacagtgaaggtggtggacg
agctcgtgaaagtgatgggccggcacaagcccgagaacatcgtgatcg
aaatggccagagagaaccagaccacccagaagggacagaagaacagcc
gcgagagaatgaagcggatcgaagagggcatcaaagagctgggcagcc
agatcctgaaagaacaccccgtggaaaacacccagctgcagaacgaga
agctgtacctgtactacctgcagaatggggggatatgtacgtggacca
ggaactggacatcaaccggctgtccgactacgatgtggacgctatcgt
gcctcagagctttctgaaggacgactccatcgacaacaaggtgctgac
cagaagcgacaagaaccggggcaagagcgacaacgtgccctccgaaga
ggtcgtgaagaagatgaagaactactggcggcagctgctgaacgccaa
gctgattacccagagaaagttcgacaatctgaccaaggccgagagagg
cggcctgagcgaactggataaggccggcttcatcaagagacagctggt
ggaaacccggcagatcacaaagcacgtggcacagatactagattcccg
aatgaatacgaaatacgacgagaacgataagctgattcgggaagtcaa
agtaatcactttaaagtcaaaattggtgtcggacttcagaaaggattt
tcaattctataaagttagggagataaataactaccaccatgcgcacga
cgcttatcttaatgccgtcgtagggaccgcactcattaagaaataccc
gaagctagaaagtgagtttgtgtatggtgattacaaagtttatgacgt
ccgtaagatgatcgcgaaaagcgaacaggagataggcaaggctacagc
caaatacttcttttattctaacattatgaatttctttaagacggaaat
cactctggcaaacggagagatacgcaaacgacctttaattgaaaccaa
tggggagacaggtgaaatcgtatgggataagggccgggacttcgcgac
ggtgagaaaagttttgtccatgccccaagtcaacatagtaaagaaaac
tgaggtgcagaccggagggttttcaaaggaatcgattcttccaaaaag
gaatagtgataagctcatcgctcgtaaaaaggactgggacccgaaaaa
gtacggtggcttcgtgagccctacagttgcctattctgtcctagtagt
ggcaaaagttgagaagggaaaatccaagaaactgaagtcagtcaaaga
attattggggataacgattatggagcgctcgtcttttgaaaagaaccc
catcgacttccttgaggcgaaaggttacaaggaagtaaaaaaggatct
cataattaaactaccaaagtatagtctgtttgagttagaaaatggccg
aaaacggatgttggctagcgccagagagcttcaaaaggggaacgaact
cgcactaccgtctaaatacgtgaatttcctgtatttagcgtcccatta
cgagaagttgaaaggttcacctgaagataacgaacagaagcaactttt
tgttgagcagcacaaacattatctcgacgaaatcatagagcaaatttc
ggaattcagtaagagagtcatcctagctgatgccaatctggacaaagt
attaagcgcatacaacaagcacagggataaacccatacgtgagcaggc
ggaaaatattatccatttgtttactcttaccaacctcggcgctccagc
cgcattcaagtattttgacacaacgatagatcgcaaagagtacagatc
taccaaggaggtgctagacgcgacactgattcaccaatccatcacggg
attatatgaaactcggatagatttgtcacagcttgggggtgacggatc
cccaaagaagaaacggaaggtgggtggaggaagtggcgggtcaggtgg
ctctagacggacactggtgaccttcaaggatgtatttgtggacttcac
cagggaggagtggaagctgctggacactgctcagcagatcgtgtacag
aaatgtgatgctggagaactataagaacctggtttccttgggttatca
gcttactaagccagatgtgatcctccggttggagaagggagaagagcc
ctcgggaggtggttcgggaggtggttcggagggtgtgcaggtgaaaag
ggtcctggagaaaagtcctgggaagctccttgtcaagatgccttttca
aacttcgccagggggcaaggctgaggggggtggggccaccacatccac
ccaggtcatggtgatcaaacgccccggcaggaagcgaaaagctgaggc
cgaccctcaggccattcccaagaaacggggccgaaagccggggagtgt
ggtggcagccgctgccgccgaggccaaaaagaaagccgtgaaggagtc
ttctatccgatctgtgcaggagacagtactccccatcaagaagcgcaa
gacccgggagggcgcgcccaagaagaagaggaaagtctccggatccgg
cgcaacaaacttctctctgctgaaacaagccggagatgtcgaagagaa
tcctggaccgaccgagtacaagcccacggtgcgcctcgccacccgcga
cgacgtccccagggccgtacgcaccctcgccgccgcgttcgccgacta
ccccgccacgcgccacaccgtcgatccggaccgccacatcgagcgggt
caccgagctgcaagaactcttcctcacgcgcgtcgggctcgacatcgg
caaggtgtgggtcgcggacgacggcgccgcggtggcggtctggaccac
gccggagagcgtcgaagcgggggcggtgttcgccgagatcggcccgcg
catggccgagttgagcggttcccggctggccgcgcagcaacagatgga
aggcctcctggcgccgcaccggcccaaggagcccgcgtggttcctggc
caccgtcggagtctcgcccgaccaccagggcaagggtctgggcagcgc
cgtcgtgctccccggagtggaggcggccgagcgcgccggggtgcccgc
cttcctggagacctccgcgccccgcaacctccccttctacgagcggct
cggcttcaccgtcaccgccgacgtcgaggtgcccgaaggaccgcgcac
ctggtgcatgacccgcaagcccggtgcctgaacgcgttaagtcgacaa
tcaacctctggattacaaaatttgtgaaagattgactggtattcttaa
ctatgttgctccttttacgctatgtggatacgctgctttaatgccttt
gtatcatgctattgcttcccgtatggctttcattttctcctccttgta
taaatcctggttgctgtctctttatgaggagttgtggcccgttgtcag
gcaacgtggcgtggtgtgcactgtgtttgctgacgcaacccccactgg
ttggggcattgccaccacctgtcagctcctttccgggactttcgcttt
ccccctccctattgccacggcggaactcatcgccgcctgccttgcccg
ctgctggacaggggctcggctgttgggcactgacaattccgtggtgtt
gtcggggaaatcatcgtcctttccttggctgctcgcctgtgttgccac
ctggattctgcgcgggacgtccttctgctacgtcccttcggccctcaa
tccagcggaccttccttcccgcggcctgctgccggctctgcggcctct
tccgcgtcttcgccttcgccctcagacgagtcggatctccctttgggc
cgcctccccgcgtcgactttaagaccaatgacttacaaggcagctgta
gatcttagccactttttaaaagaaaaggggggactggaagggctaatt
cactcccaacgaagacaagatctgctttttgcttgtactgggtctctc
tggttagaccagatctgagcctgggagctctctggctaactagggaac
ccactgcttaagcctcaataaagcttgccttgagtgcttcaagtagtg
tgtgcccgtctgttgtgtgactctggtaactagagatccctcagaccc
ttttagtcagtgtggaaaatctctagcagggcccgtttaaacccgctg
atcagcctcgactgtgccttctagttgccagccatctgttgtttgccc
ctcccccgtgccttccttgaccctggaaggtgccactcccactgtcct
ttcctaataaaatgaggaaattgcatcgcattgtctgagtaggtgtca
ttctattctggggggtggggggggcaggacagcaagggggaggattgg
gaagacaatagcaggcatgctggggatgcggtgggctctatggcttct
gaggcggaaagaaccagctggggctctagggggtatccccacgcgccc
tgtagcggcgcattaagcgcggcgggtgtggtggttacgcgcagcgtg
accgctacacttgccagcgccctagcgcccgctcctttcgctttcttc
ccttcctttctcgccacgttcgccggctttccccgtcaagctctaaat
cgggggctccctttagggttccgatttagtgctttacggcacctcgac
cccaaaaaacttgattagggtgatggttcacgtagtgggccatcgccc
tgatagacggtttttcgccctttgacgttggagtccacgttctttaat
agtggactcttgttccaaactggaacaacactcaaccctatctcggtc
tattcttttgatttataagggattttgccgatttcggcctattggtta
aaaaatgagctgatttaacaaaaatttaacgcgaattaattctgtgga
atgtgtgtcagttagggtgtggaaagtccccaggctccccagcaggca
gaagtatgcaaagcatgcatctcaattagtcagcaaccaggtgtggaa
agtccccaggctccccagcaggcagaagtatgcaaagcatgcatctca
attagtcagcaaccatagtcccgcccctaactccgcccatcccgcccc
taactccgcccagttccgcccattctccgccccatggctgactaattt
tttttatttatgcagaggccgaggccgcctctgcctctgagctattcc
agaagtagtgaggaggcttttttggaggcctaggcttttgcaaaaagc
tccctaccgtcgacctctagctagagcttggcgtaatcatggtcatag
ctgtttcctgtgtgaaattgttatccgctcacaattccacacaacata
cgagccggaagcataaagtgtaaagcctggggtgcctaatgagtgagc
taactcacattaattgcgttgcgctcactgcccgctttccagtcggga
aacctgtcgtgccagctgcattaatgaatcggccaacgcgcggggaga
ggcggtttgcgtattgggcgctcttccgcttcctcgctcactgactcg
ctgcgctcggtcgttcggctgcggcgagcggtatcagctcactcaaag
gcggtaatacggttatccacagaatcaggggataacgcaggaaagaac
atgtgagcaaaaggccagcaaaaggccaggaaccgtaaaaaggccgcg
ttgctggcgtttttccataggctccgcccccctgacgagcatcacaaa
aatcgacgctcaagtcagaggtggcgaaacccgacaggactataaaga
taccaggcgtttccccctggaagctccctcgtgcgctctcctgttccg
accctgccgcttaccggatacctgtccgcctttctcccttcgggaagc
gtggcgctttctcatagctcacgctgtaggtatctcagttcggtgtag
gtcgttcgctccaagctgggctgtgtgcacgaaccccccgttcagccc
gaccgctgcgccttatccggtaactatcgtcttgagtccaacccggta
agacacgacttatcgccactggcagcagccactggtaacaggattagc
agagcgaggtatgtaggcggtgctacagagttcttgaagtggtggcct
aactacggctacactagaagaacagtatttggtatctgcgctctgctg
aagccagttaccttcggaaaaagagttggtagctcttgatccggcaaa
caaaccaccgctggtagcggtggtttttttgtttgcaagcagcagatt
acgcgcagaaaaaaaggatctcaagaagatcctttgatcttttctacg
gggtctgacgctcagtggaacgaaaactcacgttaagggattttggtc
atgagattatcaaaaaggatcttcacctagatccttttaaattaaaaa
tgaagttttaaatcaatctaaagtatatatgagtaaacttggtctgac
agttaccaatgcttaatcagtgaggcacctatctcagcgatctgtcta
tttcgttcatccatagttgcctgactccccgtcgtgtagataactacg
atacgggagggcttaccatctggccccagtgctgcaatgataccgcga
gacccacgctcaccggctccagatttatcagcaataaaccagccagcc
ggaagggccgagcgcagaagtggtcctgcaactttatccgcctccatc
cagtctattaattgttgccgggaagctagagtaagtagttcgccagtt
aatagtttgcgcaacgttgttgccattgctacaggcatcgtggtgtca
cgctcgtcgtttggtatggcttcattcagctccggttcccaacgatca
aggcgagttacatgatcccccatgttgtgcaaaaaagcggttagctcc
ttcggtcctccgatcgttgtcagaagtaagttggccgcagtgttatca
ctcatggttatggcagcactgcataattctcttactgtcatgccatcc
gtaagatgcttttctgtgactggtgagtactcaaccaagtcattctga
gaatagtgtatgcggcgaccgagttgctcttgcccggcgtcaatacgg
gataataccgcgccacatagcagaactttaaaagtgctcatcattgga
aaacgttcttcggggcgaaaactctcaaggatcttaccgctgttgaga
tccagttcgatgtaacccactcgtgcacccaactgatcttcagcatct
tttactttcaccagcgtttctgggtgagcaaaaacaggaaggcaaaat
gccgcaaaaaagggaataagggcgacacggaaatgttgaatactcata
ctcttcctttttcaatattattgaagcatttatcagggttattgtctc
atgagcggatacatatttgaatgtatttagaaaaataaacaaataggg
gttccgcgcacatttccccgaaaagtgccacctgac.

In an aspect, a disclosed pBK1532 plasmid can comprise the sequence set forth below or a sequence having 70%, 75%, 80%, 85%, 90%, 95%, or more identity to the sequence set forth

(SEQ ID NO: 60)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggtggagtatttacggtaaactgcccacttggcagtacatcaag
tgtatcatatgccaagtacgccccctattgacgtcaatgacggtaaat
ggcccgcctggcattatgcccagtacatgaccttatgggactttccta
cttggcagtacatctacgtattagtcatcgctattaccatggtgatgc
ggttttggcagtacatcaatgggcgtggatagcggtttgactcacggg
gatttccaagtctccaccccattgacgtcaatgggagtttgttttggc
accaaaatcaacgggactttccaaaatgtcgtaacaactccgccccat
tgacgcaaatgggcggtaggcgtgtacggtgggaggtctatataagca
gcgcgttttgcctgtactgggtctctctggttagaccagatctgagcc
tgggagctctctggctaactagggaacccactgcttaagcctcaataa
agcttgccttgagtgcttcaagtagtgtgtgcccgtctgttgtgtgac
tctggtaactagagatccctcagacccttttagtcagtgtggaaaatc
tctagcagtggcgcccgaacagggacttgaaagcgaaagggaaaccag
aggagctctctcgacgcaggactcggcttgctgaagcgcgcacggcaa
gaggcgaggggcggcgactggtgagtacgccaaaaattttgactagcg
gaggctagaaggagagagatgggtgcgagagcgtcagtattaagcggg
ggagaattagatcgcgatgggaaaaaattcggttaaggccagggggaa
agaaaaaatataaattaaaacatatagtatgggcaagcagggagctag
aacgattcgcagttaatcctggcctgttagaaacatcagaaggctgta
gacaaatactgggacagctacaaccatcccttcagacaggatcagaag
aacttagatcattatataatacagtagcaaccctctattgtgtgcatc
aaaggatagagataaaagacaccaaggaagctttagacaagatagagg
aagagcaaaacaaaagtaagaccaccgcacagcaagcggccgctgatc
ttcagacctggaggaggagatatgagggacaattggagaagtgaatta
tataaatataaagtagtaaaaattgaaccattaggagtagcacccacc
aaggcaaagagaagagtggtgcagagagaaaaaagagcagtgggaata
ggagctttgttccttgggttcttgggagcagcaggaagcactatgggc
gcagcgtcaatgacgctgacggtacaggccagacaattattgtctggt
atagtgcagcagcagaacaatttgctgagggctattgaggcgcaacag
catctgttgcaactcacagtctggggcatcaagcagctccaggcaaga
atcctggctgtggaaagatacctaaaggatcaacagctcctggggatt
tggggttgctctggaaaactcatttgcaccactgctgtgccttggaat
gctagttggagtaataaatctctggaacagatttggaatcacacgacc
tggatggagtgggacagagaaattaacaattacacaagcttaatacac
tccttaattgaagaatcgcaaaaccagcaagaaaagaatgaacaagaa
ttattggaattagataaatgggcaagtttgtggaattggtttaacata
acaaattggctgtggtatataaaattattcataatgatagtaggaggc
ttggtaggtttaagaatagtttttgctgtactttctatagtgaataga
gttaggcagggatattcaccattatcgtttcagacccacctcccaacc
ccgaggggacccgacaggcccgaaggaatagaagaagaaggtggagag
agagacagagacagatccattcgattagtgaacggatcggcactgcgt
gcgccaattctgcagacaaatggcagtattcatccacaattttaaaag
aaaaggggggattggggggtacagtgcaggggaaagaatagtagacat
aatagcaacagacatacaaactaaagaattacaaaaacaaattacaaa
aattcaaaattttcgggtttattacagggacagcagagatccagtttg
gttaattaatggggggacgttaacggggcggaacggtaccgagggcct
atttcccatgattccttcatatttgcatatacgatacaaggctgttag
agagataattagaattaatttgactgtaaacacaaagatattagtaca
aaatacgtgacgtagaaagtaataatttcttgggtagtttgcagtttt
aaaattatgttttaaaatggactatcatatgcttaccgtaacttgaaa
gtatttcgatttcttggctttatatatcttgtggaaaggacgaaacac
cggggcgcggacatggaggacggttttagagctagaaatagcaagtta
aaataaggctagtccgttatcaacttgaaaaagtggcaccgagtcggt
gcttttttgaattcgctagctaggtcttgaaaggagtgggaattggct
ccggtgcccgtcagtgggcagagcgcacatcgcccacagtccccgaga
agttggggggaggggtcggcaattgatccggtgcctagagaaggtggc
gcggggtaaactgggaaagtgatgtcgtgtactggctccgcctttttc
ccgaggggggggagaaccgtatataagtgcagtagtcgccgtgaacgt
tctttttcgcaacgggtttgccgccagaacacaggaccggtgccacca
tggactataaggaccacgacggagactacaaggatcatgatattgatt
acaaagacgatgacgataagatggccccaaagaagaagcggaaggtcg
gtatccacggagtcccagcagccgacaagaagtacagcatcggcctgg
ccatcggcaccaactctgtgggctgggccgtgatcaccgacgagtaca
aggtgcccagcaagaaattcaaggtgctgggcaacaccgaccggcaca
gcatcaagaagaacctgatcggagccctgctgttcgacagcggcgaaa
cagccgaggccacccggctgaagagaaccgccagaagaagatacacca
gacggaagaaccggatctgctatctgcaagagatcttcagcaacgaga
tggccaaggtggacgacagcttcttccacagactggaagagtccttcc
tggtggaagaggataagaagcacgagcggcaccccatcttcggcaaca
tcgtggacgaggtggcctaccacgagaagtaccccaccatctaccacc
tgagaaagaaactggtggacagcaccgacaaggccgacctgcggctga
tctatctggccctggcccacatgatcaagttccggggccacttcctga
tcgagggcgacctgaaccccgacaacagcgacgtggacaagctgttca
tccagctggtgcagacctacaaccagctgttcgaggaaaaccccatca
acgccagcggcgtggacgccaaggccatcctgtctgccagactgagca
agagcagacggctggaaaatctgatcgcccagctgcccggcgagaaga
agaatggcctgttcggcaacctgattgccctgagcctgggcctgaccc
ccaacttcaagagcaacttcgacctggccgaggatgccaaactgcagc
tgagcaaggacacctacgacgacgacctggacaacctgctggcccaga
tcggcgaccagtacgccgacctgtttctggccgccaagaacctgtccg
acgccatcctgctgagcgacatcctgagagtgaacaccgagatcacca
aggcccccctgagcgcctctatgatcaagagatacgacgagcaccacc
aggacctgaccctgctgaaagctctcgtgcggcagcagctgcctgaga
agtacaaagagattttcttcgaccagagcaagaacggctacgccggct
acattgacggcggagccagccaggaagagttctacaagttcatcaagc
ccatcctggaaaagatggacggcaccgaggaactgctcgtgaagctga
acagagaggacctgctgcggaagcagcggaccttcgacaacggcagca
tcccccaccagatccacctgggagagctgcacgccattctgcggcggc
aggaagatttttacccattcctgaaggacaaccgggaaaagatcgaga
agatcctgaccttccgcatcccctactacgtgggccctctggccaggg
gaaacagcagattcgcctggatgaccagaaagagcgaggaaaccatca
ccccctggaacttcgaggaagtggtggacaagggcgcttccgcccaga
gcttcatcgagcggatgaccaacttcgataagaacctgcccaacgaga
aggtgctgcccaagcacagcctgctgtacgagtacttcaccgtgtata
acgagctgaccaaagtgaaatacgtgaccgagggaatgagaaagcccg
ccttcctgagcggcgagcagaaaaaggccatcgtggacctgctgttca
agaccaaccggaaagtgaccgtgaagcagctgaaagaggactacttca
agaaaatcgagtgcttcgactccgtggaaatctccggcgtggaagatc
ggttcaacgcctccctgggcacataccacgatctgctgaaaattatca
aggacaaggacttcctggacaatgaggaaaacgaggacattctggaag
atatcgtgctgaccctgacactgtttgaggacagagagatgatcgagg
aacggctgaaaacctatgcccacctgttcgacgacaaagtgatgaagc
agctgaagcggcggagatacaccggctggggcaggctgagccggaagc
tgatcaacggcatccgggacaagcagtccggcaagacaatcctggatt
tcctgaagtccgacggcttcgccaacagaaacttcatgcagctgatcc
acgacgacagcctgacctttaaagaggacatccagaaagcccaggtgt
ccggccagggcgatagcctgcacgagcacattgccaatctggccggca
gccccgccattaagaagggcatcctgcagacagtgaaggtggtggacg
agctcgtgaaagtgatgggccggcacaagcccgagaacatcgtgatcg
aaatggccagagagaaccagaccacccagaagggacagaagaacagcc
gcgagagaatgaagcggatcgaagagggcatcaaagagctgggcagcc
agatcctgaaagaacaccccgtggaaaacacccagctgcagaacgaga
agctgtacctgtactacctgcagaatggggggatatgtacgtggacca
ggaactggacatcaaccggctgtccgactacgatgtggacgctatcgt
gcctcagagctttctgaaggacgactccatcgacaacaaggtgctgac
cagaagcgacaagaaccggggcaagagcgacaacgtgccctccgaaga
ggtcgtgaagaagatgaagaactactggcggcagctgctgaacgccaa
gctgattacccagagaaagttcgacaatctgaccaaggccgagagagg
cggcctgagcgaactggataaggccggcttcatcaagagacagctggt
ggaaacccggcagatcacaaagcacgtggcacagatactagattcccg
aatgaatacgaaatacgacgagaacgataagctgattcgggaagtcaa
agtaatcactttaaagtcaaaattggtgtcggacttcagaaaggattt
tcaattctataaagttagggagataaataactaccaccatgcgcacga
cgcttatcttaatgccgtcgtagggaccgcactcattaagaaataccc
gaagctagaaagtgagtttgtgtatggtgattacaaagtttatgacgt
ccgtaagatgatcgcgaaaagcgaacaggagataggcaaggctacagc
caaatacttcttttattctaacattatgaatttctttaagacggaaat
cactctggcaaacggagagatacgcaaacgacctttaattgaaaccaa
tggggagacaggtgaaatcgtatgggataagggccgggacttcgcgac
ggtgagaaaagttttgtccatgccccaagtcaacatagtaaagaaaac
tgaggtgcagaccggagggttttcaaaggaatcgattcttccaaaaag
gaatagtgataagctcatcgctcgtaaaaaggactgggacccgaaaaa
gtacggtggcttcgtgagccctacagttgcctattctgtcctagtagt
ggcaaaagttgagaagggaaaatccaagaaactgaagtcagtcaaaga
attattggggataacgattatggagcgctcgtcttttgaaaagaaccc
catcgacttccttgaggcgaaaggttacaaggaagtaaaaaaggatct
cataattaaactaccaaagtatagtctgtttgagttagaaaatggccg
aaaacggatgttggctagcgccagagagcttcaaaaggggaacgaact
cgcactaccgtctaaatacgtgaatttcctgtatttagcgtcccatta
cgagaagttgaaaggttcacctgaagataacgaacagaagcaactttt
tgttgagcagcacaaacattatctcgacgaaatcatagagcaaatttc
ggaattcagtaagagagtcatcctagctgatgccaatctggacaaagt
attaagcgcatacaacaagcacagggataaacccatacgtgagcaggc
ggaaaatattatccatttgtttactcttaccaacctcggcgctccagc
cgcattcaagtattttgacacaacgatagatcgcaaagagtacagatc
taccaaggaggtgctagacgcgacactgattcaccaatccatcacggg
attatatgaaactcggatagatttgtcacagcttgggggtgacggatc
cccaaagaagaaacggaaggtgggtggaggaagtggcgggtcaggtgg
ctctagacggacactggtgaccttcaaggatgtatttgtggacttcac
cagggaggagtggaagctgctggacactgctcagcagatcgtgtacag
aaatgtgatgctggagaactataagaacctggtttccttgggttatca
gcttactaagccagatgtgatcctccggttggagaagggagaagagcc
ctcgggaggtggttcgggaggtggttcggagggtgtgcaggtgaaaag
ggtcctggagaaaagtcctgggaagctccttgtcaagatgccttttca
aacttcgccagggggcaaggctgaggggggtggggccaccacatccac
ccaggtcatggtgatcaaacgccccggcaggaagcgaaaagctgaggc
cgaccctcaggccattcccaagaaacggggccgaaagccggggagtgt
ggtggcagccgctgccgccgaggccaaaaagaaagccgtgaaggagtc
ttctatccgatctgtgcaggagacagtactccccatcaagaagcgcaa
gacccgggagggcgcgcccaagaagaagaggaaagtctccggatccgg
cgcaacaaacttctctctgctgaaacaagccggagatgtcgaagagaa
tcctggaccgaccgagtacaagcccacggtgcgcctcgccacccgcga
cgacgtccccagggccgtacgcaccctcgccgccgcgttcgccgacta
ccccgccacgcgccacaccgtcgatccggaccgccacatcgagcgggt
caccgagctgcaagaactcttcctcacgcgcgtcgggctcgacatcgg
caaggtgtgggtcgcggacgacggcgccgcggtggcggtctggaccac
gccggagagcgtcgaagcgggggcggtgttcgccgagatcggcccgcg
catggccgagttgagcggttcccggctggccgcgcagcaacagatgga
aggcctcctggcgccgcaccggcccaaggagcccgcgtggttcctggc
caccgtcggagtctcgcccgaccaccagggcaagggtctgggcagcgc
cgtcgtgctccccggagtggaggcggccgagcgcgccggggtgcccgc
cttcctggagacctccgcgccccgcaacctccccttctacgagcggct
cggcttcaccgtcaccgccgacgtcgaggtgcccgaaggaccgcgcac
ctggtgcatgacccgcaagcccggtgcctgaacgcgttaagtcgacaa
tcaacctctggattacaaaatttgtgaaagattgactggtattcttaa
ctatgttgctccttttacgctatgtggatacgctgctttaatgccttt
gtatcatgctattgcttcccgtatggctttcattttctcctccttgta
taaatcctggttgctgtctctttatgaggagttgtggcccgttgtcag
gcaacgtggcgtggtgtgcactgtgtttgctgacgcaacccccactgg
ttggggcattgccaccacctgtcagctcctttccgggactttcgcttt
ccccctccctattgccacggcggaactcatcgccgcctgccttgcccg
ctgctggacaggggctcggctgttgggcactgacaattccgtggtgtt
gtcggggaaatcatcgtcctttccttggctgctcgcctgtgttgccac
ctggattctgcgcgggacgtccttctgctacgtcccttcggccctcaa
tccagcggaccttccttcccgcggcctgctgccggctctgcggcctct
tccgcgtcttcgccttcgccctcagacgagtcggatctccctttgggc
cgcctccccgcgtcgactttaagaccaatgacttacaaggcagctgta
gatcttagccactttttaaaagaaaaggggggactggaagggctaatt
cactcccaacgaagacaagatctgctttttgcttgtactgggtctctc
tggttagaccagatctgagcctgggagctctctggctaactagggaac
ccactgcttaagcctcaataaagcttgccttgagtgcttcaagtagtg
tgtgcccgtctgttgtgtgactctggtaactagagatccctcagaccc
ttttagtcagtgtggaaaatctctagcagggcccgtttaaacccgctg
atcagcctcgactgtgccttctagttgccagccatctgttgtttgccc
ctcccccgtgccttccttgaccctggaaggtgccactcccactgtcct
ttcctaataaaatgaggaaattgcatcgcattgtctgagtaggtgtca
ttctattctggggggtggggggggcaggacagcaagggggaggattgg
gaagacaatagcaggcatgctggggatgcggtgggctctatggcttct
gaggcggaaagaaccagctggggctctagggggtatccccacgcgccc
tgtagcggcgcattaagcgcggcgggtgtggtggttacgcgcagcgtg
accgctacacttgccagcgccctagcgcccgctcctttcgctttcttc
ccttcctttctcgccacgttcgccggctttccccgtcaagctctaaat
cgggggctccctttagggttccgatttagtgctttacggcacctcgac
cccaaaaaacttgattagggtgatggttcacgtagtgggccatcgccc
tgatagacggtttttcgccctttgacgttggagtccacgttctttaat
agtggactcttgttccaaactggaacaacactcaaccctatctcggtc
tattcttttgatttataagggattttgccgatttcggcctattggtta
aaaaatgagctgatttaacaaaaatttaacgcgaattaattctgtgga
atgtgtgtcagttagggtgtggaaagtccccaggctccccagcaggca
gaagtatgcaaagcatgcatctcaattagtcagcaaccaggtgtggaa
agtccccaggctccccagcaggcagaagtatgcaaagcatgcatctca
attagtcagcaaccatagtcccgcccctaactccgcccatcccgcccc
taactccgcccagttccgcccattctccgccccatggctgactaattt
tttttatttatgcagaggccgaggccgcctctgcctctgagctattcc
agaagtagtgaggaggcttttttggaggcctaggcttttgcaaaaagc
tccctaccgtcgacctctagctagagcttggcgtaatcatggtcatag
ctgtttcctgtgtgaaattgttatccgctcacaattccacacaacata
cgagccggaagcataaagtgtaaagcctggggtgcctaatgagtgagc
taactcacattaattgcgttgcgctcactgcccgctttccagtcggga
aacctgtcgtgccagctgcattaatgaatcggccaacgcgcggggaga
ggcggtttgcgtattgggcgctcttccgcttcctcgctcactgactcg
ctgcgctcggtcgttcggctgcggcgagcggtatcagctcactcaaag
gcggtaatacggttatccacagaatcaggggataacgcaggaaagaac
atgtgagcaaaaggccagcaaaaggccaggaaccgtaaaaaggccgcg
ttgctggcgtttttccataggctccgcccccctgacgagcatcacaaa
aatcgacgctcaagtcagaggtggcgaaacccgacaggactataaaga
taccaggcgtttccccctggaagctccctcgtgcgctctcctgttccg
accctgccgcttaccggatacctgtccgcctttctcccttcgggaagc
gtggcgctttctcatagctcacgctgtaggtatctcagttcggtgtag
gtcgttcgctccaagctgggctgtgtgcacgaaccccccgttcagccc
gaccgctgcgccttatccggtaactatcgtcttgagtccaacccggta
agacacgacttatcgccactggcagcagccactggtaacaggattagc
agagcgaggtatgtaggcggtgctacagagttcttgaagtggtggcct
aactacggctacactagaagaacagtatttggtatctgcgctctgctg
aagccagttaccttcggaaaaagagttggtagctcttgatccggcaaa
caaaccaccgctggtagcggtggtttttttgtttgcaagcagcagatt
acgcgcagaaaaaaaggatctcaagaagatcctttgatcttttctacg
gggtctgacgctcagtggaacgaaaactcacgttaagggattttggtc
atgagattatcaaaaaggatcttcacctagatccttttaaattaaaaa
tgaagttttaaatcaatctaaagtatatatgagtaaacttggtctgac
agttaccaatgcttaatcagtgaggcacctatctcagcgatctgtcta
tttcgttcatccatagttgcctgactccccgtcgtgtagataactacg
atacgggagggcttaccatctggccccagtgctgcaatgataccgcga
gacccacgctcaccggctccagatttatcagcaataaaccagccagcc
ggaagggccgagcgcagaagtggtcctgcaactttatccgcctccatc
cagtctattaattgttgccgggaagctagagtaagtagttcgccagtt
aatagtttgcgcaacgttgttgccattgctacaggcatcgtggtgtca
cgctcgtcgtttggtatggcttcattcagctccggttcccaacgatca
aggcgagttacatgatcccccatgttgtgcaaaaaagcggttagctcc
ttcggtcctccgatcgttgtcagaagtaagttggccgcagtgttatca
ctcatggttatggcagcactgcataattctcttactgtcatgccatcc
gtaagatgcttttctgtgactggtgagtactcaaccaagtcattctga
gaatagtgtatgcggcgaccgagttgctcttgcccggcgtcaatacgg
gataataccgcgccacatagcagaactttaaaagtgctcatcattgga
aaacgttcttcggggcgaaaactctcaaggatcttaccgctgttgaga
tccagttcgatgtaacccactcgtgcacccaactgatcttcagcatct
tttactttcaccagcgtttctgggtgagcaaaaacaggaaggcaaaat
gccgcaaaaaagggaataagggcgacacggaaatgttgaatactcata
ctcttcctttttcaatattattgaagcatttatcagggttattgtctc
atgagcggatacatatttgaatgtatttagaaaaataaacaaataggg
gttccgcgcacatttccccgaaaagtgccacctgac.

In an aspect, a disclosed pBK1536 plasmid can comprise the sequence set forth below or a sequence having 70%, 75%, 80%, 85%, 90%, 95%, or more identity to the sequence set forth below:

(SEQ ID NO: 61)
gtcgacggatcgggagatctcccgatcccctatggtgcactctcagta
caatctgctctgatgccgcatagttaagccagtatctgctccctgctt
gtgtgttggaggtcgctgagtagtgcgcgagcaaaatttaagctacaa
caaggcaaggcttgaccgacaattgcatgaagaatctgcttagggtta
ggcgttttgcgctgcttcgcgatgtacgggccagatatacgcgttgac
attgattattgactagttattaatagtaatcaattacggggtcattag
ttcatagcccatatatggagttccgcgttacataacttacggtaaatg
gcccgcctggctgaccgcccaacgacccccgcccattgacgtcaataa
tgacgtatgttcccatagtaacgccaatagggactttccattgacgtc
aatgggtggagtatttacggtaaactgcccacttggcagtacatcaag
tgtatcatatgccaagtacgccccctattgacgtcaatgacggtaaat
ggcccgcctggcattatgcccagtacatgaccttatgggactttccta
cttggcagtacatctacgtattagtcatcgctattaccatggtgatgc
ggttttggcagtacatcaatgggcgtggatagcggtttgactcacggg
gatttccaagtctccaccccattgacgtcaatgggagtttgttttggc
accaaaatcaacgggactttccaaaatgtcgtaacaactccgccccat
tgacgcaaatgggcggtaggcgtgtacggtgggaggtctatataagca
gcgcgttttgcctgtactgggtctctctggttagaccagatctgagcc
tgggagctctctggctaactagggaacccactgcttaagcctcaataa
agcttgccttgagtgcttcaagtagtgtgtgcccgtctgttgtgtgac
tctggtaactagagatccctcagacccttttagtcagtgtggaaaatc
tctagcagtggcgcccgaacagggacttgaaagcgaaagggaaaccag
aggagctctctcgacgcaggactcggcttgctgaagcgcgcacggcaa
gaggcgaggggcggcgactggtgagtacgccaaaaattttgactagcg
gaggctagaaggagagagatgggtgcgagagcgtcagtattaagcggg
ggagaattagatcgcgatgggaaaaaattcggttaaggccagggggaa
agaaaaaatataaattaaaacatatagtatgggcaagcagggagctag
aacgattcgcagttaatcctggcctgttagaaacatcagaaggctgta
gacaaatactgggacagctacaaccatcccttcagacaggatcagaag
aacttagatcattatataatacagtagcaaccctctattgtgtgcatc
aaaggatagagataaaagacaccaaggaagctttagacaagatagagg
aagagcaaaaaaaagtaagaccaccgcacagcaagcggccgctgatct
tcagacctggaggaggagatatgagggacaattggagaagtgaattat
ataaatataaagtagtaaaaattgaaccattaggagtagcacccacca
aggcaaagagaagagtggtgcagagagaaaaaagagcagtgggaatag
gagctttgttccttgggttcttgggagcagcaggaagcactatgggcg
cagcgtcaatgacgctgacggtacaggccagacaattattgtctggta
tagtgcagcagcagaacaatttgctgagggctattgaggcgcaacagc
atctgttgcaactcacagtctggggcatcaagcagctccaggcaagaa
tcctggctgtggaaagatacctaaaggatcaacagctcctggggattt
ggggttgctctggaaaactcatttgcaccactgctgtgccttggaatg
ctagttggagtaataaatctctggaacagatttggaatcacacgacct
ggatggagtgggacagagaaattaacaattacacaagcttaatacact
ccttaattgaagaatcgcaaaaccagcaagaaaagaatgaacaagaat
tattggaattagataaatgggcaagtttgtggaattggtttaacataa
caaattggctgtggtatataaaattattcataatgatagtaggaggct
tggtaggtttaagaatagtttttgctgtactttctatagtgaatagag
ttaggcagggatattcaccattatcgtttcagacccacctcccaaccc
cgaggggacccgacaggcccgaaggaatagaagaagaaggtggagaga
gagacagagacagatccattcgattagtgaacggatcggcactgcgtg
cgccaattctgcagacaaatggcagtattcatccacaattttaaaaga
aaaggggggattggggggtacagtgcaggggaaagaatagtagacata
atagcaacagacatacaaactaaagaattacaaaaacaaattacaaaa
attcaaaattttcgggtttattacagggacagcagagatccagtttgg
ttaattaatggggggacgttaacggggcggaacggtaccgagggccta
tttcccatgattccttcatatttgcatatacgatacaaggctgttaga
gagataattagaattaatttgactgtaaacacaaagatattagtacaa
aatacgtgacgtagaaagtaataatttcttgggtagtttgcagtttta
aaattatgttttaaaatggactatcatatgcttaccgtaacttgaaag
tatttcgatttcttggctttatatatcttgtggaaaggacgaaacacc
ggagacgtgtacacgtctctgttttagagctagaaatagcaagttaaa
ataaggctagtccgttatcaacttgaaaaagtggcaccgagtcggtgc
ttttttgaattcgctagctaggtcttgaaaggagtgggaattggctcc
ggtgcccgtcagtgggcagagcgcacatcgcccacagtccccgagaag
ttggggggaggggtcggcaattgatccggtgcctagagaaggtggcgc
ggggtaaactgggaaagtgatgtcgtgtactggctccgcctttttccc
gaggggggggagaaccgtatataagtgcagtagtcgccgtgaacgttc
tttttcgcaacgggtttgccgccagaacacaggaccggtgccaccatg
gactataaggaccacgacggagactacaaggatcatgatattgattac
aaagacgatgacgataagatggccccaaagaagaagcggaaggtcggt
atccacggagtcccagcagccgacaagaagtacagcatcggcctggcc
atcggcaccaactctgtgggctgggccgtgatcaccgacgagtacaag
gtgcccagcaagaaattcaaggtgctgggcaacaccgaccggcacagc
atcaagaagaacctgatcggagccctgctgttcgacagcggcgaaaca
gccgaggccacccggctgaagagaaccgccagaagaagatacaccaga
cggaagaaccggatctgctatctgcaagagatcttcagcaacgagatg
gccaaggtggacgacagcttcttccacagactggaagagtccttcctg
gtggaagaggataagaagcacgagcggcaccccatcttcggcaacatc
gtggacgaggtggcctaccacgagaagtaccccaccatctaccacctg
agaaagaaactggtggacagcaccgacaaggccgacctgcggctgatc
tatctggccctggcccacatgatcaagttccggggccacttcctgatc
gagggcgacctgaaccccgacaacagcgacgtggacaagctgttcatc
cagctggtgcagacctacaaccagctgttcgaggaaaaccccatcaac
gccagcggcgtggacgccaaggccatcctgtctgccagactgagcaag
agcagacggctggaaaatctgatcgcccagctgcccggcgagaagaag
aatggcctgttcggcaacctgattgccctgagcctgggcctgaccccc
aacttcaagagcaacttcgacctggccgaggatgccaaactgcagctg
agcaaggacacctacgacgacgacctggacaacctgctggcccagatc
ggcgaccagtacgccgacctgtttctggccgccaagaacctgtccgac
gccatcctgctgagcgacatcctgagagtgaacaccgagatcaccaag
gcccccctgagcgcctctatgatcaagagatacgacgagcaccaccag
gacctgaccctgctgaaagctctcgtgcggcagcagctgcctgagaag
tacaaagagattttcttcgaccagagcaagaacggctacgccggctac
attgacggcggagccagccaggaagagttctacaagttcatcaagccc
atcctggaaaagatggacggcaccgaggaactgctcgtgaagctgaac
agagaggacctgctgcggaagcagcggaccttcgacaacggcagcatc
ccccaccagatccacctgggagagctgcacgccattctgcggcggcag
gaagatttttacccattcctgaaggacaaccgggaaaagatcgagaag
atcctgaccttccgcatcccctactacgtgggccctctggccagggga
aacagcagattcgcctggatgaccagaaagagcgaggaaaccatcacc
ccctggaacttcgaggaagtggtggacaagggcgcttccgcccagagc
ttcatcgagcggatgaccaacttcgataagaacctgcccaacgagaag
gtgctgcccaagcacagcctgctgtacgagtacttcaccgtgtataac
gagctgaccaaagtgaaatacgtgaccgagggaatgagaaagcccgcc
ttcctgagcggcgagcagaaaaaggccatcgtggacctgctgttcaag
accaaccggaaagtgaccgtgaagcagctgaaagaggactacttcaag
aaaatcgagtgcttcgactccgtggaaatctccggcgtggaagatcgg
ttcaacgcctccctgggcacataccacgatctgctgaaaattatcaag
gacaaggacttcctggacaatgaggaaaacgaggacattctggaagat
atcgtgctgaccctgacactgtttgaggacagagagatgatcgaggaa
cggctgaaaacctatgcccacctgttcgacgacaaagtgatgaagcag
ctgaagcggcggagatacaccggctggggcaggctgagccggaagctg
atcaacggcatccgggacaagcagtccggcaagacaatcctggatttc
ctgaagtccgacggcttcgccaacagaaacttcatgcagctgatccac
gacgacagcctgacctttaaagaggacatccagaaagcccaggtgtcc
ggccagggcgatagcctgcacgagcacattgccaatctggccggcagc
cccgccattaagaagggcatcctgcagacagtgaaggtggtggacgag
ctcgtgaaagtgatgggccggcacaagcccgagaacatcgtgatcgaa
atggccagagagaaccagaccacccagaagggacagaagaacagccgc
gagagaatgaagcggatcgaagagggcatcaaagagctgggcagccag
atcctgaaagaacaccccgtggaaaacacccagctgcagaacgagaag
ctgtacctgtactacctgcagaatggggggatatgtacgtggaccagg
aactggacatcaaccggctgtccgactacgatgtggacgctatcgtgc
ctcagagctttctgaaggacgactccatcgacaacaaggtgctgacca
gaagcgacaagaaccggggcaagagcgacaacgtgccctccgaagagg
tcgtgaagaagatgaagaactactggcggcagctgctgaacgccaagc
tgattacccagagaaagttcgacaatctgaccaaggccgagagaggcg
gcctgagcgaactggataaggccggcttcatcaagagacagctggtgg
aaacccggcagatcacaaagcacgtggcacagatactagattcccgaa
tgaatacgaaatacgacgagaacgataagctgattcgggaagtcaaag
taatcactttaaagtcaaaattggtgtcggacttcagaaaggattttc
aattctataaagttagggagataaataactaccaccatgcgcacgacg
cttatcttaatgccgtcgtagggaccgcactcattaagaaatacccga
agctagaaagtgagtttgtgtatggtgattacaaagtttatgacgtcc
gtaagatgatcgcgaaaagcgaacaggagataggcaaggctacagcca
aatacttcttttattctaacattatgaatttctttaagacggaaatca
ctctggcaaacggagagatacgcaaacgacctttaattgaaaccaatg
gggagacaggtgaaatcgtatgggataagggccgggacttcgcgacgg
tgagaaaagttttgtccatgccccaagtcaacatagtaaagaaaactg
aggtgcagaccggagggttttcaaaggaatcgattcttccaaaaagga
atagtgataagctcatcgctcgtaaaaaggactgggacccgaaaaagt
acggtggcttcgtgagccctacagttgcctattctgtcctagtagtgg
caaaagttgagaagggaaaatccaagaaactgaagtcagtcaaagaat
tattggggataacgattatggagcgctcgtcttttgaaaagaacccca
tcgacttccttgaggcgaaaggttacaaggaagtaaaaaaggatctca
taattaaactaccaaagtatagtctgtttgagttagaaaatggccgaa
aacggatgttggctagcgccagagagcttcaaaaggggaacgaactcg
cactaccgtctaaatacgtgaatttcctgtatttagcgtcccattacg
agaagttgaaaggttcacctgaagataacgaacagaagcaactttttg
ttgagcagcacaaacattatctcgacgaaatcatagagcaaatttcgg
aattcagtaagagagtcatcctagctgatgccaatctggacaaagtat
taagcgcatacaacaagcacagggataaacccatacgtgagcaggcgg
aaaatattatccatttgtttactcttaccaacctcggcgctccagccg
cattcaagtattttgacacaacgatagatcgcaaagagtacagatcta
ccaaggaggtgctagacgcgacactgattcaccaatccatcacgggat
tatatgaaactcggatagatttgtcacagcttgggggtgacggatccc
caaagaagaaacggaaggtgggtggaggaagtggcgggtcaggtggct
ctagacggacactggtgaccttcaaggatgtatttgtggacttcacca
gggaggagtggaagctgctggacactgctcagcagatcgtgtacagaa
atgtgatgctggagaactataagaacctggtttccttgggttatcagc
ttactaagccagatgtgatcctccggttggagaagggagaagagccct
cgggaggtggttcgggaggtggttcggagggtgtgcaggtgaaaaggg
tcctggagaaaagtcctgggaagctccttgtcaagatgccttttcaaa
cttcgccagggggcaaggctgaggggggtggggccaccacatccaccc
aggtcatggtgatcaaacgccccggcaggaagcgaaaagctgaggccg
accctcaggccattcccaagaaacggggccgaaagccggggagtgtgg
tggcagccgctgccgccgaggccaaaaagaaagccgtgaaggagtctt
ctatccgatctgtgcaggagacagtactccccatcaagaagcgcaaga
cccgggagggcgcgcccaagaagaagaggaaagtctccggatccggcg
caacaaacttctctctgctgaaacaagccggagatgtcgaagagaatc
ctggaccgaccgagtacaagcccacggtgcgcctcgccacccgcgacg
acgtccccagggccgtacgcaccctcgccgccgcgttcgccgactacc
ccgccacgcgccacaccgtcgatccggaccgccacatcgagcgggtca
ccgagctgcaagaactcttcctcacgcgcgtcgggctcgacatcggca
aggtgtgggtcgcggacgacggcgccgcggtggcggtctggaccacgc
cggagagcgtcgaagcgggggcggtgttcgccgagatcggcccgcgca
tggccgagttgagcggttcccggctggccgcgcagcaacagatggaag
gcctcctggcgccgcaccggcccaaggagcccgcgtggttcctggcca
ccgtcggagtctcgcccgaccaccagggcaagggtctgggcagcgccg
tcgtgctccccggagtggaggcggccgagcgcgccggggtgcccgcct
tcctggagacctccgcgccccgcaacctccccttctacgagcggctcg
gcttcaccgtcaccgccgacgtcgaggtgcccgaaggaccgcgcacct
ggtgcatgacccgcaagcccggtgcctgaacgcgttaagtcgacaatc
aacctctggattacaaaatttgtgaaagattgactggtattcttaact
atgttgctccttttacgctatgtggatacgctgctttaatgcctttgt
atcatgctattgcttcccgtatggctttcattttctcctccttgtata
aatcctggttgctgtctctttatgaggagttgtggcccgttgtcaggc
aacgtggcgtggtgtgcactgtgtttgctgacgcaacccccactggtt
ggggcattgccaccacctgtcagctcctttccgggactttcgctttcc
ccctccctattgccacggcggaactcatcgccgcctgccttgcccgct
gctggacaggggctcggctgttgggcactgacaattccgtggtgttgt
cggggaaatcatcgtcctttccttggctgctcgcctgtgttgccacct
ggattctgcgcgggacgtccttctgctacgtcccttcggccctcaatc
cagcggaccttccttcccgcggcctgctgccggctctgcggcctcttc
cgcgtcttcgccttcgccctcagacgagtcggatctccctttgggccg
cctccccgcgtcgactttaagaccaatgacttacaaggcagctgtaga
tcttagccactttttaaaagaaaaggggggactggaagggctaattca
ctcccaacgaagacaagatctgctttttgcttgtactgggtctctctg
gttagaccagatctgagcctgggagctctctggctaactagggaaccc
actgcttaagcctcaataaagcttgccttgagtgcttcaagtagtgtg
tgcccgtctgttgtgtgactctggtaactagagatccctcagaccctt
ttagtcagtgtggaaaatctctagcagggcccgtttaaacccgctgat
cagcctcgactgtgccttctagttgccagccatctgttgtttgcccct
cccccgtgccttccttgaccctggaaggtgccactcccactgtccttt
cctaataaaatgaggaaattgcatcgcattgtctgagtaggtgtcatt
ctattctggggggtggggggggcaggacagcaagggggaggattggga
agacaatagcaggcatgctggggatgcggtgggctctatggcttctga
ggcggaaagaaccagctggggctctagggggtatccccacgcgccctg
tagcggcgcattaagcgcggcgggtgtggtggttacgcgcagcgtgac
cgctacacttgccagcgccctagcgcccgctcctttcgctttcttccc
ttcctttctcgccacgttcgccggctttccccgtcaagctctaaatcg
ggggctccctttagggttccgatttagtgctttacggcacctcgaccc
caaaaaacttgattagggtgatggttcacgtagtgggccatcgccctg
atagacggtttttcgccctttgacgttggagtccacgttctttaatag
tggactcttgttccaaactggaacaacactcaaccctatctcggtcta
ttcttttgatttataagggattttgccgatttcggcctattggttaaa
aaatgagctgatttaacaaaaatttaacgcgaattaattctgtggaat
gtgtgtcagttagggtgtggaaagtccccaggctccccagcaggcaga
agtatgcaaagcatgcatctcaattagtcagcaaccaggtgtggaaag
tccccaggctccccagcaggcagaagtatgcaaagcatgcatctcaat
tagtcagcaaccatagtcccgcccctaactccgcccatcccgccccta
actccgcccagttccgcccattctccgccccatggctgactaattttt
tttatttatgcagaggccgaggccgcctctgcctctgagctattccag
aagtagtgaggaggcttttttggaggcctaggcttttgcaaaaagctc
cctaccgtcgacctctagctagagcttggcgtaatcatggtcatagct
gtttcctgtgtgaaattgttatccgctcacaattccacacaacatacg
agccggaagcataaagtgtaaagcctggggtgcctaatgagtgagcta
actcacattaattgcgttgcgctcactgcccgctttccagtcgggaaa
cctgtcgtgccagctgcattaatgaatcggccaacgcgcggggagagg
cggtttgcgtattgggcgctcttccgcttcctcgctcactgactcgct
gcgctcggtcgttcggctgcggcgagcggtatcagctcactcaaaggc
ggtaatacggttatccacagaatcaggggataacgcaggaaagaacat
gtgagcaaaaggccagcaaaaggccaggaaccgtaaaaaggccgcgtt
gctggcgtttttccataggctccgcccccctgacgagcatcacaaaaa
tcgacgctcaagtcagaggtggcgaaacccgacaggactataaagata
ccaggcgtttccccctggaagctccctcgtgcgctctcctgttccgac
cctgccgcttaccggatacctgtccgcctttctcccttcgggaagcgt
ggcgctttctcatagctcacgctgtaggtatctcagttcggtgtaggt
cgttcgctccaagctgggctgtgtgcacgaaccccccgttcagcccga
ccgctgcgccttatccggtaactatcgtcttgagtccaacccggtaag
acacgacttatcgccactggcagcagccactggtaacaggattagcag
agcgaggtatgtaggcggtgctacagagttcttgaagtggtggcctaa
ctacggctacactagaagaacagtatttggtatctgcgctctgctgaa
gccagttaccttcggaaaaagagttggtagctcttgatccggcaaaca
aaccaccgctggtagcggtggtttttttgtttgcaagcagcagattac
gcgcagaaaaaaaggatctcaagaagatcctttgatcttttctacggg
gtctgacgctcagtggaacgaaaactcacgttaagggattttggtcat
gagattatcaaaaaggatcttcacctagatccttttaaattaaaaatg
aagttttaaatcaatctaaagtatatatgagtaaacttggtctgacag
ttaccaatgcttaatcagtgaggcacctatctcagcgatctgtctatt
tcgttcatccatagttgcctgactccccgtcgtgtagataactacgat
acgggagggcttaccatctggccccagtgctgcaatgataccgcgaga
cccacgctcaccggctccagatttatcagcaataaaccagccagccgg
aagggccgagcgcagaagtggtcctgcaactttatccgcctccatcca
gtctattaattgttgccgggaagctagagtaagtagttcgccagttaa
tagtttgcgcaacgttgttgccattgctacaggcatcgtggtgtcacg
ctcgtcgtttggtatggcttcattcagctccggttcccaacgatcaag
gcgagttacatgatcccccatgttgtgcaaaaaagcggttagctcctt
cggtcctccgatcgttgtcagaagtaagttggccgcagtgttatcact
catggttatggcagcactgcataattctcttactgtcatgccatccgt
aagatgcttttctgtgactggtgagtactcaaccaagtcattctgaga
atagtgtatgcggcgaccgagttgctcttgcccggcgtcaatacggga
taataccgcgccacatagcagaactttaaaagtgctcatcattggaaa
acgttcttcggggcgaaaactctcaaggatcttaccgctgttgagatc
cagttcgatgtaacccactcgtgcacccaactgatcttcagcatcttt
tactttcaccagcgtttctgggtgagcaaaaacaggaaggcaaaatgc
cgcaaaaaagggaataagggcgacacggaaatgttgaatactcatact
cttcctttttcaatattattgaagcatttatcagggttattgtctcat
gagcggatacatatttgaatgtatttagaaaaataaacaaataggggt
tccgcgcacatttccccgaaaagtgccacctgac.

### C. Methods of Administering Precision Gene Therapy

Disclosed herein is a method of administering precision gene therapy, the method comprising contacting one or more cells with a therapeutically effective amount of a disclosed isolated nucleic acid molecule, and reducing the activity and/or expression of APOE in one or more cells.

Disclosed herein is a method of administering precision gene therapy, the method comprising contacting one or more cells with a therapeutically effective amount of a disclosed isolated nucleic acid molecule, and reducing the activity and/or expression of APOE e4 in one or more cells.

Disclosed herein is a method of administering precision gene therapy, the method comprising contacting one or more cells with a therapeutically effective amount of a viral vector, wherein the viral vector comprises an isolated nucleic acid molecule comprising a nucleic acid sequence encoding (i) a fusion protein and (ii) at least one guide RNA, wherein the fusion protein comprises a Cas endonuclease and a polypeptide having an enzymatic activity, and reducing the activity and/or expression of APOE in one or more cells.

Disclosed herein is a method of administering precision gene therapy, the method comprising contacting one or more cells with a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, and reducing the activity and/or expression of APOE in one or more cells.

Disclosed herein is a method of administering precision gene therapy, the method comprising contacting one or more cells with a therapeutically effective amount of a viral vector, wherein the viral vector comprises an isolated nucleic acid molecule comprising a nucleic acid sequence encoding (i) a fusion protein and (ii) at least one guide RNA, wherein the fusion protein comprises a Cas endonuclease and a polypeptide having an enzymatic activity, and reducing the activity and/or expression of the APOE e4 allele in one or more cells.

Disclosed herein is a method of administering precision gene therapy, the method comprising contacting one or more cells with a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, and reducing the activity and/or expression of the APOE e4 allele in one or more cells.

In an aspect of a disclosed method, increased APOE expression and/or activity can be mediated by a coding mutation in exon 4, gene dysregulation, or a combination thereof.

In an aspect, a disclosed method can reduce expression and/or activity of APOE regardless of the subject's genotype.

In an aspect, the disclosed cells can be neurons such as, for example, cholinergic neurons. In an aspect, the disclosed cells can be in a subject.

In an aspect, a disclosed viral vector can be a lentiviral vector. In an aspect of a disclosed method, a disclosed viral vector can comprise one or more promoters operably linked to the isolated nucleic acid molecule. In an aspect, a disclosed promoter can drive the expression of a gRNA, the Cas9 endonuclease, a polypeptide, or a combination thereof. In an aspect, a disclosed promoter can be a hU6 promoter and a disclosed hU6 promoter can drive expression of a gRNA. In an aspect, a promoter can be an EFS-NC promoter and a disclosed EFS-NC promoter can drive expression of the Cas endonuclease. In an aspect, a disclosed promoter can comprise a hU6 promoter, an EFS-NC promoter, or a combination thereof.

In an aspect of a disclosed method, a disclosed viral vector can comprise one or more promoters operably linked to the isolated nucleic acid molecule and one or more regulatory elements. Regulatory elements are known in the art and can comprise one or more of the following: a Sp1 responsive element, a p2A signal, a woodchuck hepatitis virus post-transcriptional regulatory element, a Phi signal-packaging signal, a rev responsive element, a 5′-LTR, and a 3′-LTR. In an aspect, a disclosed viral vector can comprise two Sp1 response elements, a p2A signal, a woodchuck hepatitis virus post-transcriptional regulatory element, a Phi signal-packaging signal, a rev responsive element, a 5′-LTR, and a 3′-LTR.

In an aspect, a disclosed Cas endonuclease can comprise Cas9, SpCas9, SaCas9, a variant Cas9, a dCas, or a dCas9. In an aspect, a disclosed Cas9 can comprise the sequence set forth in SEQ ID NO:64 or SEQ ID NO:65. In an aspect, a disclosed Cas9 can have a sequence having at least 75%, at least 80%, at least 85%, at least 90%, or at least 95% identity to the sequence set forth in SEQ ID NO:64 or SEQ ID NO:65 or a fragment thereof.

In an aspect, a disclosed variant Cas9 can comprise VQR, EQR, or VRER. In an aspect a disclosed VRER can comprise the sequence set forth in SEQ ID NO:15. In an aspect, a disclosed VRER can have a sequence having at least 75%, at least 80%, at least 85%, at least 90%, or at least 95% identity to the sequence set forth in SEQ ID NO:15 or a fragment thereof. In an aspect, a disclosed dCas can comprise dVQR, dEQR, or dVRER. In an aspect, a disclosed dCas can comprise the sequence set forth in SEQ ID NO:16. In an aspect, a disclosed dCas9 can have a sequence having at least 75%, at least 80%, at least 85%, at least 90%, or at least 95% identity to the sequence set forth in SEQ ID NO:16 or a fragment thereof. A SpCas9 (3′NGG-PAM sequence) can comprise SpCas9 VQR (3′NGAN or 3′NGNG), SpCas9 EQR (3′NGAG), or SpCas9 VRER (3′NGCG).

In an aspect, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof. In an aspect, a disclosed encoded polypeptide can be histone deacetylase or heterochromatin protein 1. In an aspect, a disclosed encoded polypeptide can comprise transcription repression activity. In an aspect, a disclosed DNMT3A can have the amino acid sequence set forth in SEQ ID NO:17 or the nucleotide sequence set forth in SEQ ID NO:18. In an aspect, a disclosed DNMT3A can have a sequence having at least 75%, at least 80%, at least 85%, at least 90%, or at least 95% identity to the sequence set forth in SEQ ID NO:17 or SEQ ID NO:18 or a fragment thereof.

In an aspect, at least one encoded polypeptide can comprise Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed TRD of MeCP2 can comprise the nucleotide sequence set forth in SEQ ID NO:57 or the amino acid sequence set forth in SEQ ID NO:58. In an aspect, a disclosed TRD of MeCP2 can have a sequence having at least 75%, at least 80%, at least 85%, at least 90%, or at least 95% identity to the sequence set forth in SEQ ID NO:57 or SEQ ID NO:58 or a fragment thereof. In an aspect, a disclosed KRAB-MeCP2 can comprise the nucleotide sequence set forth in SEQ ID NO:62 or the amino acid sequence set forth in SEQ ID NO:63. In an aspect, a disclosed KRAB-MeCP2 can have a sequence having at least 75%, at least 80%, at least 85%, at least 90%, or at least 95% identity to the sequence set forth in SEQ ID NO:62 or SEQ ID NO:63 or a fragment thereof.

In an aspect, a disclosed gRNA can be designed to target exon 4 of the APOE gene or designed to target a protospacer-adjacent motif (PAM) created by a SNP rs429358 in exon 4 of the APOE gene. In an aspect, a disclosed gRNA can have the sequence set forth in any one of SEQ ID NO:05-SEQ ID NO:14, SEQ ID NO:25-SEQ ID NO:28, SEQ ID NO:39-SEQ ID NO:42, and SEQ ID NO:51-SEQ ID NO:52.

In an aspect, a disclosed Cas endonuclease can be fused to a disclosed polypeptide having an enzymatic activity. In an aspect, a disclosed Cas endonuclease can be dCas9 and the polypeptide can be DNMT3A. In an aspect, a dCas9-DNMT3A fusion protein can have the amino acid sequence set forth SEQ ID NO:19. In an aspect, a dCas9-DNMT3A fusion protein can be encoded by the sequence set forth in SEQ ID NO:20. In an aspect, a disclosed dCas9-DNMT3A fusion protein can have a sequence having at least 75%, at least 80%, at least 85%, at least 90%, or at least 95% identity to the sequence set forth in SEQ ID NO: 19 or SEQ ID NO:20 or a fragment thereof. In an aspect, a disclosed Cas endonuclease can be dVRER and the polypeptide can be DNMT3A. In an aspect, a disclosed dVRER-DNMT3A fusion protein can have the amino acid sequence set forth SEQ ID NO:38. In an aspect, a disclosed dVRER-DNMT3A fusion protein can be encoded by the sequence set forth in SEQ ID NO:37. In an aspect, a disclosed dVRER-DNMT3A fusion protein can have a sequence having at least 75%, at least 80%, at least 85%, at least 90%, or at least 95% identity to the sequence set forth in SEQ ID NO:38 or SEQ ID NO:37 or a fragment thereof.

In an aspect, a disclosed Cas endonuclease can be dCas9 and the polypeptide can be Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed Cas endonuclease can be dVRER and the polypeptide can be Krüppel-associated box (KRAB), the transcription repression domain (TRD) of Methyl-CpG Binding Protein 2 (MeCP2), or a fusion of KRAB-MeCP2 (KRAB-MeCP2).

In an aspect of a disclosed method, a subject can be a human. In an aspect, a subject can be suspected of having or can be diagnosed with having Alzheimer's disease (such as, for example, LOAD). In an aspect, a disclosed subject can be symptomatic or asymptomatic.

In an aspect, a disclosed method can comprise reducing the pathological phenotype associated with Alzheimer's disease. In an aspect, reducing the pathological phenotype associated with Alzheimer's disease can comprise reducing the A042/40 ratio and reducing the level of Tau. In an aspect, a disclosed method can comprise diagnosing the subject with Alzheimer's disease.

In an aspect, a disclosed method can comprise repeating one or more steps of the method and/or modifying one or more steps of the method.

In an aspect of a disclosed method, administering a disclosed viral vector can comprise intravenous administration, intracerebral administration, intra-CSF administration, intracerebroventricular (ICV) administration, intraventricular administration, intra-cistema magna (ICM) administration, intraparenchymal administration, intrathecal (lumbar, cistemal, or both) administration, or any combination thereof.

In an aspect, a disclosed method can comprise administering to the subject a therapeutically effective amount of a therapeutic agent, an effective amount of an immune modulator, or a combination thereof.

Disclosed herein is a method of administering precision gene therapy, the method comprising administering to a subject in need thereof a therapeutically effective amount of a viral vector, wherein the viral vector comprises an isolated nucleic acid molecule comprising a nucleic acid sequence encoding (i) a fusion protein and (ii) at least one guide RNA, wherein the fusion protein comprises a Cas endonuclease and a polypeptide having an enzymatic activity, and reducing expression of the APOE e4 allele.

Disclosed herein is a method of administering precision gene therapy, the method comprising administering to a subject in need thereof a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, and reducing expression of the APOE e4 allele.

In an aspect, the disclosed cells can be neurons such as, for example, cholinergic neurons. In an aspect, the disclosed cells can be in a subject.

In an aspect, a disclosed viral vector can be a lentiviral vector. In an aspect of a disclosed method, a disclosed viral vector can comprise one or more promoters operably linked to the isolated nucleic acid molecule. In an aspect, a disclosed promoter can drive the expression of a gRNA, the Cas9 endonuclease, a polypeptide, or a combination thereof. In an aspect, a disclosed promoter can be a hU6 promoter and a disclosed hU6 promoter can drive expression of a gRNA. In an aspect, a disclosed promoter can be an EFS-NC promoter and a disclosed EFS-NC promoter can drive expression of the Cas endonuclease. In an aspect, a disclosed promoter can comprise a hU6 promoter, an EFS-NC promoter, or a combination thereof.

In an aspect of a disclosed method, a disclosed viral vector can comprise one or more promoters operably linked to the isolated nucleic acid molecule and one or more regulatory elements. Regulatory elements are known in the art and can comprise one or more of the following: a Sp1 responsive element, a p2A signal, a woodchuck hepatitis virus post-transcriptional regulatory element, a Phi signal-packaging signal, a rev responsive element, a 5′-LTR, and a 3′-LTR. In an aspect, a disclosed viral vector can comprise two Sp1 response elements, a p2A signal, a woodchuck hepatitis virus post-transcriptional regulatory element, a Phi signal-packaging signal, a rev responsive element, a 5′-LTR, and a 3′-LTR.

In an aspect, a disclosed Cas endonuclease can comprise Cas9, SpCas9, SaCas9, a variant Cas9, a dCas, or a dCas9. In an aspect, a disclosed Cas9 can comprise the sequence set forth in SEQ ID NO:64 or SEQ ID NO:65. In an aspect, a disclosed Cas9 can have a sequence having at least 75%, at least 80%, at least 85%, at least 90%, or at least 95% identity to the sequence set forth in SEQ ID NO:64 or SEQ ID NO:65 or a fragment thereof.

In an aspect, a disclosed variant Cas9 can comprise VQR, EQR, or VRER. In an aspect a disclosed VRER can comprise the sequence set forth in SEQ ID NO:15. In an aspect, a disclosed VRER can have a sequence having at least 75%, at least 80%, at least 85%, at least 90%, or at least 95% identity to the sequence set forth in SEQ ID NO:15 or a fragment thereof. In an aspect, a disclosed dCas can comprise dVQR, dEQR, or dVRER. In an aspect, a disclosed dCas can comprise the sequence set forth in SEQ ID NO:16. In an aspect, a disclosed dCas9 can have a sequence having at least 75%, at least 80%, at least 85%, at least 90%, or at least 95% identity to the sequence set forth in SEQ ID NO:16 or a fragment thereof. A SpCas9 (3′NGG-PAM sequence) can comprise SpCas9 VQR (3′NGAN or 3′NGNG), SpCas9 EQR (3′NGAG), or SpCas9 VRER (3′NGCG).

In an aspect, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof. In an aspect, a disclosed encoded polypeptide can be histone deacetylase or heterochromatin protein 1. In an aspect, a disclosed encoded polypeptide can comprise transcription repression activity. In an aspect, a disclosed DNMT3A can have the amino acid sequence set forth in SEQ ID NO:17 or the nucleotide sequence set forth in SEQ ID NO:18. In an aspect, a disclosed DNMT3A can have a sequence having at least 75%, at least 80%, at least 85%, at least 90%, or at least 95% identity to the sequence set forth in SEQ ID NO:17 or SEQ ID NO:18 or a fragment thereof.

In an aspect, at least one encoded polypeptide can comprise Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed TRD of MeCP2 can comprise the nucleotide sequence set forth in SEQ ID NO:57 or the amino acid sequence set forth in SEQ ID NO:58. In an aspect, a disclosed TRD of MeCP2 can have a sequence having at least 75%, at least 80%, at least 85%, at least 90%, or at least 95% identity to the sequence set forth in SEQ ID NO:57 or SEQ ID NO:58 or a fragment thereof. In an aspect, a disclosed KRAB-MeCP2 can comprise the nucleotide sequence set forth in SEQ ID NO:62 or the amino acid sequence set forth in SEQ ID NO:63. In an aspect, a disclosed KRAB-MeCP2 can have a sequence having at least 75%, at least 80%, at least 85%, at least 90%, or at least 95% identity to the sequence set forth in SEQ ID NO:62 or SEQ ID NO:63 or a fragment thereof.

In an aspect, a disclosed gRNA can be designed to target exon 4 of the APOE gene or designed to target a protospacer-adjacent motif (PAM) created by a SNP rs429358 in exon 4 of the APOE gene. In an aspect, a disclosed gRNA can have the sequence set forth in any one of SEQ ID NO:05-SEQ ID NO:14, SEQ ID NO:25-SEQ ID NO:28, SEQ ID NO:39-SEQ ID NO:42, and SEQ ID NO:51-SEQ ID NO:52.

In an aspect, a disclosed Cas endonuclease can be fused to a disclosed polypeptide having an enzymatic activity. In an aspect, a disclosed Cas endonuclease can be dCas9 and the polypeptide can be DNMT3A. In an aspect, a dCas9-DNMT3A fusion protein can have the amino acid sequence set forth SEQ ID NO:19. In an aspect, a dCas9-DNMT3A fusion protein can be encoded by the sequence set forth in SEQ ID NO:20. In an aspect, a disclosed dCas9-DNMT3A fusion protein can have a sequence having at least 75%, at least 80%, at least 85%, at least 90%, or at least 95% identity to the sequence set forth in SEQ ID NO: 19 or SEQ ID NO:20 or a fragment thereof.

In an aspect, a disclosed Cas endonuclease can be dVRER and a disclosed polypeptide can be DNMT3A. In an aspect, a disclosed dVRER-DNMT3A fusion protein can have the amino acid sequence set forth SEQ ID NO:38. In an aspect, a disclosed dVRER-DNMT3A fusion protein can be encoded by the sequence set forth in SEQ ID NO:37. In an aspect, a disclosed dVRER-DNMT3A fusion protein can have a sequence having at least 75%, at least 80%, at least 85%, at least 90%, or at least 95% identity to the sequence set forth in SEQ ID NO:38 or SEQ ID NO:37 or a fragment thereof.

In an aspect, a disclosed Cas endonuclease can be dCas9 and the polypeptide can be Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed Cas endonuclease can be dVRER and the polypeptide can be Krüppel-associated box (KRAB), the transcription repression domain (TRD) of Methyl-CpG Binding Protein 2 (MeCP2), or a fusion of KRAB-MeCP2 (KRAB-MeCP2).

In an aspect of a disclosed method, a subject can be a human. In an aspect, a subject can be suspected of having or can be diagnosed with having Alzheimer's disease (such as, for example, LOAD). In an aspect, a disclosed subject can be symptomatic or asymptomatic.

In an aspect, a disclosed method can comprise reducing the pathological phenotype associated with Alzheimer's disease. In an aspect, reducing the pathological phenotype associated with Alzheimer's disease can comprise reducing the A042/40 ratio and reducing the level of Tau. In an aspect, a disclosed method can comprise diagnosing the subject with Alzheimer's disease.

In an aspect, a disclosed method can comprise repeating one or more steps of the method and/or modifying one or more steps of the method.

In an aspect of a disclosed method, administering a disclosed vector can comprise intravenous administration, intracerebral administration, intra-CSF administration, intracerebroventricular (ICV) administration, intraventricular administration, intra-cistema magna (ICM) administration, intraparenchymal administration, intrathecal (lumbar, cistemal, or both) administration, or any combination thereof.

In an aspect, a disclosed method can comprise administering to the subject a therapeutically effective amount of a therapeutic agent, an effective amount of an immune modulator, or a combination thereof.

### D. Methods of Treating and/or Preventing Alzheimer's Disease Progression

Disclosed herein is a method of treating and/or preventing Alzheimer's disease progression in a subject, the method comprising administering to a subject in need thereof a therapeutically effective amount of a viral vector, wherein the viral vector comprises an isolated nucleic acid molecule comprising a nucleic acid sequence encoding (i) a fusion protein comprising a Cas endonuclease and a polypeptide having an enzymatic activity and (ii) at least one guide RNA, and reducing expression of APOE, thereby reducing the pathological phenotype associated with Alzheimer's disease.

Disclosed herein is a method of treating and/or preventing Alzheimer's disease progression in a subject, the method comprising reducing the pathological phenotype associated with Alzheimer's disease by administering to a subject in need thereof a therapeutically effective amount of a viral vector, wherein the viral vector comprises an isolated nucleic acid molecule comprising a nucleic acid sequence encoding (i) a fusion protein comprising a Cas endonuclease and a polypeptide having an enzymatic activity and (ii) at least one guide RNA, and reducing expression of APOE.

Disclosed herein is a method of treating and/or preventing Alzheimer's disease progression in a subject, the method comprising administering to a subject in need thereof a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, and reducing expression of APOE, thereby reducing the pathological phenotype associated with Alzheimer's disease.

Disclosed herein is a method of treating and/or preventing Alzheimer's disease progression in a subject, the method comprising reducing the pathological phenotype associated with Alzheimer's disease by administering to a subject in need thereof a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, and reducing expression of APOE.

Disclosed herein is a method of treating and/or preventing Alzheimer's disease progression in a subject, the method comprising administering to a subject in need thereof a therapeutically effective amount of a viral vector, wherein the viral vector comprises an isolated nucleic acid molecule comprising a nucleic acid sequence encoding (i) a fusion protein comprising a Cas endonuclease and a polypeptide having an enzymatic activity and (ii) at least one guide RNA, and reducing expression of the APOE e4 allele, thereby reducing the pathological phenotype associated with Alzheimer's disease.

Disclosed herein is a method of treating and/or preventing Alzheimer's disease progression in a subject, the method comprising reducing the pathological phenotype associated with Alzheimer's disease by administering to a subject in need thereof a therapeutically effective amount of a viral vector, wherein the viral vector comprises an isolated nucleic acid molecule comprising a nucleic acid sequence encoding (i) a fusion protein comprising a Cas endonuclease and a polypeptide having an enzymatic activity and (ii) at least one guide RNA, and reducing expression of the APOE e4 allele.

Disclosed herein is a method of treating and/or preventing Alzheimer's disease progression in a subject, the method comprising administering to a subject in need thereof a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, and reducing expression of the APOE e4 allele, thereby reducing the pathological phenotype associated with Alzheimer's disease.

Disclosed herein is a method of treating and/or preventing Alzheimer's disease progression in a subject, the method comprising reducing the pathological phenotype associated with Alzheimer's disease by administering to a subject in need thereof a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, and reducing expression of the APOE e4 allele.

In an aspect of a disclosed method, increased APOE expression and/or activity can be mediated by a coding mutation in exon 4, gene dysregulation, or a combination thereof.

In an aspect, a disclosed method can reduce expression and/or activity of APOE regardless of the subject's genotype.

In an aspect, a disclosed viral vector can be a lentiviral vector. In an aspect of a disclosed method, a disclosed viral vector can comprise one or more promoters operably linked to the isolated nucleic acid molecule. In an aspect, a disclosed promoter can drive the expression of a gRNA, the Cas9 endonuclease, a polypeptide, or a combination thereof. In an aspect, a disclosed promoter can be a hU6 promoter and a disclosed hU6 promoter can drive expression of a gRNA. In an aspect, a disclosed promoter can be an EFS-NC promoter and a disclosed EFS-NC promoter can drive expression of the Cas endonuclease. In an aspect, a disclosed promoter can comprise a hU6 promoter, an EFS-NC promoter, or a combination thereof.

In an aspect of a disclosed method, a disclosed viral vector can comprise one or more promoters operably linked to the isolated nucleic acid molecule and one or more regulatory elements. Regulatory elements are known in the art and can comprise one or more of the following: a Sp1 responsive element, a p2A signal, a woodchuck hepatitis virus post-transcriptional regulatory element, a Phi signal-packaging signal, a rev responsive element, a 5′-LTR, and a 3′-LTR. In an aspect, a disclosed viral vector can comprise two Sp1 response elements, a p2A signal, a woodchuck hepatitis virus post-transcriptional regulatory element, a Phi signal-packaging signal, a rev responsive element, a 5′-LTR, and a 3′-LTR.

In an aspect, a disclosed Cas endonuclease can comprise Cas9, SpCas9, SaCas9, a variant Cas9, a dCas, or a dCas9. In an aspect, a disclosed Cas9 can comprise the sequence set forth in SEQ ID NO:64 or SEQ ID NO:65. In an aspect, a disclosed Cas9 can have a sequence having at least 75%, at least 80%, at least 85%, at least 90%, or at least 95% identity to the sequence set forth in SEQ ID NO:64 or SEQ ID NO:65 or a fragment thereof.

In an aspect, a disclosed variant Cas9 can comprise VQR, EQR, or VRER. In an aspect a disclosed VRER can comprise the sequence set forth in SEQ ID NO:15. In an aspect, a disclosed VRER can have a sequence having at least 75%, at least 80%, at least 85%, at least 90%, or at least 95% identity to the sequence set forth in SEQ ID NO:15 or a fragment thereof. In an aspect, a disclosed dCas can comprise dVQR, dEQR, or dVRER. In an aspect, a disclosed dCas can comprise the sequence set forth in SEQ ID NO:16. In an aspect, a disclosed dCas9 can have a sequence having at least 75%, at least 80%, at least 85%, at least 90%, or at least 95% identity to the sequence set forth in SEQ ID NO:16 or a fragment thereof. A SpCas9 (3′NGG-PAM sequence) can comprise SpCas9 VQR (3′NGAN or 3′NGNG), SpCas9 EQR (3′NGAG), or SpCas9 VRER (3′NGCG).

In an aspect, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof. In an aspect, a disclosed encoded polypeptide can be histone deacetylase or heterochromatin protein 1. In an aspect, a disclosed encoded polypeptide can comprise transcription repression activity. In an aspect, a disclosed DNMT3A can have the amino acid sequence set forth in SEQ ID NO:17 or the nucleotide sequence set forth in SEQ ID NO:18. In an aspect, a disclosed DNMT3A can have a sequence having at least 75%, at least 80%, at least 85%, at least 90%, or at least 95% identity to the sequence set forth in SEQ ID NO:17 or SEQ ID NO:18 or a fragment thereof.

In an aspect, at least one encoded polypeptide can comprise Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed TRD of MeCP2 can comprise the nucleotide sequence set forth in SEQ ID NO:57 or the amino acid sequence set forth in SEQ ID NO:58. In an aspect, a disclosed TRD of MeCP2 can have a sequence having at least 75%, at least 80%, at least 85%, at least 90%, or at least 95% identity to the sequence set forth in SEQ ID NO:57 or SEQ ID NO:58 or a fragment thereof. In an aspect, a disclosed KRAB-MeCP2 can comprise the nucleotide sequence set forth in SEQ ID NO:62 or the amino acid sequence set forth in SEQ ID NO:63. In an aspect, a disclosed KRAB-MeCP2 can have a sequence having at least 75%, at least 80%, at least 85%, at least 90%, or at least 95% identity to the sequence set forth in SEQ ID NO:62 or SEQ ID NO:63 or a fragment thereof.

In an aspect, a disclosed gRNA can be designed to target exon 4 of the APOE gene or designed to target a protospacer-adjacent motif (PAM) created by a SNP rs429358 in exon 4 of the APOE gene. In an aspect, a disclosed gRNA can have the sequence set forth in any one of SEQ ID NO:05-SEQ ID NO:14, SEQ ID NO:25-SEQ ID NO:28, SEQ ID NO:39-SEQ ID NO:42, and SEQ ID NO:51-SEQ ID NO:52.

In an aspect, a disclosed Cas endonuclease can be fused to a disclosed polypeptide having an enzymatic activity. In an aspect, a disclosed Cas endonuclease can be dCas9 and the polypeptide can be DNMT3A. In an aspect, a dCas9-DNMT3A fusion protein can have the amino acid sequence set forth SEQ ID NO:19. In an aspect, a dCas9-DNMT3A fusion protein can be encoded by the sequence set forth in SEQ ID NO:20. In an aspect, a disclosed Cas endonuclease can be dVRER and the polypeptide can be DNMT3A. In an aspect, a disclosed dVRER-DNMT3A fusion protein can have the amino acid sequence set forth SEQ ID NO:38. In an aspect, a disclosed dVRER-DNMT3A fusion protein can be encoded by the sequence set forth in SEQ ID NO:37. In an aspect, a disclosed dVRER-DNMT3A fusion protein can have a sequence having at least 75%, at least 80%, at least 85%, at least 90%, or at least 95% identity to the sequence set forth in SEQ ID NO:38 or SEQ ID NO:37 or a fragment thereof.

In an aspect, a disclosed Cas endonuclease can be dCas9 and the polypeptide can be Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed Cas endonuclease can be dVRER and the polypeptide can be Krüppel-associated box (KRAB), the transcription repression domain (TRD) of Methyl-CpG Binding Protein 2 (MeCP2), or a fusion of KRAB-MeCP2 (KRAB-MeCP2).

In an aspect of a disclosed method, a subject can be a human. In an aspect, a subject can be suspected of having or can be diagnosed with having Alzheimer's disease (such as, for example, LOAD). In an aspect, a disclosed subject can be symptomatic or asymptomatic. In an aspect, a disclosed method can comprise reducing the pathological phenotype associated with Alzheimer's disease. In an aspect, reducing the pathological phenotype associated with Alzheimer's disease can comprise reducing the A042/40 ratio and reducing the level of Tau. In an aspect, a disclosed method can comprise diagnosing the subject with Alzheimer's disease.

In an aspect, a disclosed method can comprise repeating one or more steps of the method and/or modifying one or more steps of the method.

In an aspect of a disclosed method, administering a disclosed vector can comprise intravenous administration, intracerebral administration, intra-CSF administration, intracerebroventricular (ICV) administration, intraventricular administration, intra-cistema magna (ICM) administration, intraparenchymal administration, intrathecal (lumbar, cistemal, or both) administration, or any combination thereof.

In an aspect, a disclosed method can comprise administering to the subject a therapeutically effective amount of a therapeutic agent, an effective amount of an immune modulator, or a combination thereof.

In an aspect, a disclosed method can comprise administering one or more additional therapeutic agents. Additional therapeutic agents can comprise any disclosed therapeutic agents. A therapeutic agent can be any that effects a desired clinical outcome in a subject having a Alzheimer's disease, suspected of having Alzheimer's disease, and/or likely to develop or acquire Alzheimer's disease. In an aspect, a disclosed therapeutic agent can be an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed therapeutic agent can comprise an isolated nucleic acid molecule encoding a protein that is deficient or absent in the subject. In an aspect, a disclosed therapeutic agent can be a biologically active agent, a pharmaceutically active agent, an anti-bacterial agent, an anti-fungal agent, a corticosteroid, an analgesic, an immunostimulant, an immune-based product, or any combination thereof.

### E. Methods of Reducing Activity and/or Expression of APOE

Disclosed herein is a method of reducing expression of APOE, the method comprising administering to a subject in need thereof a therapeutically effective amount of a viral vector, wherein the viral vector comprises an isolated nucleic acid molecule comprising a nucleic acid sequence encoding (i) a fusion protein comprising a Cas endonuclease and a polypeptide having an enzymatic activity and (ii) at least one guide RNA, thereby reducing expression of APOE.

Disclosed herein is a method of reducing expression of APOE, the method comprising administering to a subject in need thereof a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, thereby reducing expression of APOE.

In an aspect of a disclosed method, increased APOE expression and/or activity can be mediated by a coding mutation in exon 4, gene dysregulation, or a combination thereof.

In an aspect, a disclosed viral vector can be a lentiviral vector. In an aspect of a disclosed method, a disclosed viral vector can comprise one or more promoters operably linked to the isolated nucleic acid molecule. In an aspect, a disclosed promoter can drive the expression of a gRNA, the Cas9 endonuclease, a polypeptide, or a combination thereof. In an aspect, a disclosed promoter can be a hU6 promoter and a disclosed hU6 promoter can drive expression of a gRNA. In an aspect, a disclosed promoter can be an EFS-NC promoter and a disclosed EFS-NC promoter can drive expression of the Cas endonuclease. In an aspect, a disclosed promoter can comprise a hU6 promoter, an EFS-NC promoter, or a combination thereof.

In an aspect of a disclosed method, a disclosed viral vector can comprise one or more promoters operably linked to the isolated nucleic acid molecule and one or more regulatory elements. Regulatory elements are known in the art and can comprise one or more of the following: a Sp1 responsive element, a p2A signal, a woodchuck hepatitis virus post-transcriptional regulatory element, a Phi signal-packaging signal, a rev responsive element, a 5′-LTR, and a 3′-LTR. In an aspect, a disclosed viral vector can comprise two Sp1 response elements, a p2A signal, a woodchuck hepatitis virus post-transcriptional regulatory element, a Phi signal-packaging signal, a rev responsive element, a 5′-LTR, and a 3′-LTR.

In an aspect, a disclosed Cas endonuclease can comprise Cas9, SpCas9, SaCas9, a variant Cas9, a dCas, or a dCas9. In an aspect, a disclosed Cas9 can comprise the sequence set forth in SEQ ID NO:64 or SEQ ID NO:65. In an aspect, a disclosed Cas9 can have a sequence having at least 75%, at least 80%, at least 85%, at least 90%, or at least 95% identity to the sequence set forth in SEQ ID NO:64 or SEQ ID NO:65 or a fragment thereof.

In an aspect, a disclosed variant Cas9 can comprise VQR, EQR, or VRER. In an aspect a disclosed VRER can comprise the sequence set forth in SEQ ID NO:15. In an aspect, a disclosed VRER can have a sequence having at least 75%, at least 80%, at least 85%, at least 90%, or at least 95% identity to the sequence set forth in SEQ ID NO:15 or a fragment thereof. In an aspect, a disclosed dCas can comprise dVQR, dEQR, or dVRER. In an aspect, a disclosed dCas can comprise the sequence set forth in SEQ ID NO:16. In an aspect, a disclosed dCas9 can have a sequence having at least 75%, at least 80%, at least 85%, at least 90%, or at least 95% identity to the sequence set forth in SEQ ID NO:16 or a fragment thereof. A SpCas9 (3′NGG-PAM sequence) can comprise SpCas9 VQR (3′NGAN or 3′NGNG), SpCas9 EQR (3′NGAG), or SpCas9 VRER (3′NGCG).

In an aspect, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof. In an aspect, a disclosed encoded polypeptide can be histone deacetylase or heterochromatin protein 1. In an aspect, a disclosed encoded polypeptide can comprise transcription repression activity. In an aspect, a disclosed DNMT3A can have the amino acid sequence set forth in SEQ ID NO:17 or the nucleotide sequence set forth in SEQ ID NO:18. In an aspect, a disclosed DNMT3A can have a sequence having at least 75%, at least 80%, at least 85%, at least 90%, or at least 95% identity to the sequence set forth in SEQ ID NO:17 or SEQ ID NO:18 or a fragment thereof.

In an aspect, at least one encoded polypeptide can comprise Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed TRD of MeCP2 can comprise the nucleotide sequence set forth in SEQ ID NO:57 or the amino acid sequence set forth in SEQ ID NO:58. In an aspect, a disclosed TRD of MeCP2 can have a sequence having at least 75%, at least 80%, at least 85%, at least 90%, or at least 95% identity to the sequence set forth in SEQ ID NO:57 or SEQ ID NO:58 or a fragment thereof. In an aspect, a disclosed KRAB-MeCP2 can comprise the nucleotide sequence set forth in SEQ ID NO:62 or the amino acid sequence set forth in SEQ ID NO:63. In an aspect, a disclosed KRAB-MeCP2 can have a sequence having at least 75%, at least 80%, at least 85%, at least 90%, or at least 95% identity to the sequence set forth in SEQ ID NO:62 or SEQ ID NO:63 or a fragment thereof.

In an aspect, a disclosed gRNA can be designed to target exon 4 of the APOE gene or designed to target a protospacer-adjacent motif (PAM) created by a SNP rs429358 in exon 4 of the APOE gene. In an aspect, a disclosed gRNA can have the sequence set forth in any one of SEQ ID NO:05-SEQ ID NO:14, SEQ ID NO:25-SEQ ID NO:28, SEQ ID NO:39-SEQ ID NO:42, and SEQ ID NO:51-SEQ ID NO:52.

In an aspect, a disclosed Cas endonuclease can be fused to a disclosed polypeptide having an enzymatic activity. In an aspect, a disclosed Cas endonuclease can be dCas9 and the polypeptide can be DNMT3A. In an aspect, a dCas9-DNMT3A fusion protein can have the amino acid sequence set forth SEQ ID NO:19. In an aspect, a dCas9-DNMT3A fusion protein can be encoded by the sequence set forth in SEQ ID NO:20. In an aspect, a disclosed Cas endonuclease can be dVRER and the polypeptide can be DNMT3A. In an aspect, a disclosed dVRER-DNMT3A fusion protein can have the amino acid sequence set forth SEQ ID NO:38. In an aspect, a disclosed dVRER-DNMT3A fusion protein can be encoded by the sequence set forth in SEQ ID NO:37. In an aspect, a disclosed dVRER-DNMT3A fusion protein can have a sequence having at least 75%, at least 80%, at least 85%, at least 90%, or at least 95% identity to the sequence set forth in SEQ ID NO:38 or SEQ ID NO:37 or a fragment thereof.

In an aspect, a disclosed Cas endonuclease can be dCas9 and the polypeptide can be Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed Cas endonuclease can be dVRER and the polypeptide can be Krüppel-associated box (KRAB), the transcription repression domain (TRD) of Methyl-CpG Binding Protein 2 (MeCP2), or a fusion of KRAB-MeCP2 (KRAB-MeCP2).

In an aspect of a disclosed method, a subject can be a human. In an aspect, a subject can be suspected of having or can be diagnosed with having Alzheimer's disease (such as, for example, LOAD). In an aspect, a disclosed subject can be symptomatic or asymptomatic. In an aspect, a disclosed method can comprise reducing the pathological phenotype associated with Alzheimer's disease. In an aspect, reducing the pathological phenotype associated with Alzheimer's disease can comprise reducing the A042/40 ratio and reducing the level of Tau. In an aspect, a disclosed method can comprise diagnosing the subject with Alzheimer's disease.

In an aspect, a disclosed method can comprise repeating one or more steps of the method and/or modifying one or more steps of the method.

In an aspect of a disclosed method, administering a disclosed vector can comprise intravenous administration, intracerebral administration, intra-CSF administration, intracerebroventricular (ICV) administration, intraventricular administration, intra-cistema magna (ICM) administration, intraparenchymal administration, intrathecal (lumbar, cisternal, or both) administration, or any combination thereof.

In an aspect, a disclosed method can comprise administering to the subject a therapeutically effective amount of a therapeutic agent, an effective amount of an immune modulator, or a combination thereof.

### F. Methods of Reducing Activity and/or Expression of APOE e4

Disclosed herein is a method of reducing expression of APOE e4, the method comprising administering to a subject in need thereof a therapeutically effective amount of a viral vector, wherein the viral vector comprises an isolated nucleic acid molecule comprising a nucleic acid sequence encoding (i) a fusion protein comprising a Cas endonuclease and a polypeptide having an enzymatic activity and (ii) at least one guide RNA, thereby reducing expression of the APOE e4 allele.

Disclosed herein is a method of reducing expression of APOE e4, the method comprising administering to a subject in need thereof a therapeutically effective amount of a viral vector, wherein the viral vector comprises an isolated nucleic acid molecule comprising a nucleic acid sequence encoding (i) a Cas endonuclease, (ii) at least one polypeptide having an enzymatic activity, and (iii) at least one guide RNA, thereby reducing expression of the APOE e4 allele.

In an aspect of a disclosed method, increased APOE expression and/or activity can be mediated by a coding mutation in exon 4, gene dysregulation, or a combination thereof.

In an aspect, a disclosed viral vector can be a lentiviral vector. In an aspect of a disclosed method, a disclosed viral vector can comprise one or more promoters operably linked to the isolated nucleic acid molecule. In an aspect, a disclosed promoter can drive the expression of a gRNA, the Cas9 endonuclease, a polypeptide, or a combination thereof. In an aspect, a disclosed promoter can be a hU6 promoter and a disclosed hU6 promoter can drive expression of a gRNA. In an aspect, a disclosed promoter can be an EFS-NC promoter and a disclosed EFS-NC promoter can drive expression of the Cas endonuclease. In an aspect, a disclosed promoter can comprise a hU6 promoter, an EFS-NC promoter, or a combination thereof.

In an aspect of a disclosed method, a disclosed viral vector can comprise one or more promoters operably linked to the isolated nucleic acid molecule and one or more regulatory elements. Regulatory elements are known in the art and can comprise one or more of the following: a Sp1 responsive element, a p2A signal, a woodchuck hepatitis virus post-transcriptional regulatory element, a Phi signal-packaging signal, a rev responsive element, a 5′-LTR, and a 3′-LTR. In an aspect, a disclosed viral vector can comprise two Sp1 response elements, a p2A signal, a woodchuck hepatitis virus post-transcriptional regulatory element, a Phi signal-packaging signal, a rev responsive element, a 5′-LTR, and a 3′-LTR.

In an aspect, a disclosed Cas endonuclease can comprise Cas9, SpCas9, SaCas9, a variant Cas9, a dCas, or a dCas9. In an aspect, a disclosed Cas9 can comprise the sequence set forth in SEQ ID NO:64 or SEQ ID NO:65. In an aspect, a disclosed Cas9 can have a sequence having at least 75%, at least 80%, at least 85%, at least 90%, or at least 95% identity to the sequence set forth in SEQ ID NO:64 or SEQ ID NO:65 or a fragment thereof.

In an aspect, a disclosed variant Cas9 can comprise VQR, EQR, or VRER. In an aspect a disclosed VRER can comprise the sequence set forth in SEQ ID NO:15. In an aspect, a disclosed VRER can have a sequence having at least 75%, at least 80%, at least 85%, at least 90%, or at least 95% identity to the sequence set forth in SEQ ID NO:15 or a fragment thereof. In an aspect, a disclosed dCas can comprise dVQR, dEQR, or dVRER. In an aspect, a disclosed dCas can comprise the sequence set forth in SEQ ID NO:16. In an aspect, a disclosed dCas9 can have a sequence having at least 75%, at least 80%, at least 85%, at least 90%, or at least 95% identity to the sequence set forth in SEQ ID NO:16 or a fragment thereof. A SpCas9 (3′NGG-PAM sequence) can comprise SpCas9 VQR (3′NGAN or 3′NGNG), SpCas9 EQR (3′NGAG), or SpCas9 VRER (3′NGCG).

In an aspect, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof. In an aspect, a disclosed encoded polypeptide can be histone deacetylase or heterochromatin protein 1. In an aspect, a disclosed encoded polypeptide can comprise transcription repression activity. In an aspect, a disclosed DNMT3A can have the amino acid sequence set forth in SEQ ID NO:17 or the nucleotide sequence set forth in SEQ ID NO:18. In an aspect, a disclosed DNMT3A can have a sequence having at least 75%, at least 80%, at least 85%, at least 90%, or at least 95% identity to the sequence set forth in SEQ ID NO:17 or SEQ ID NO:18 or a fragment thereof.

In an aspect, at least one encoded polypeptide can comprise Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed TRD of MeCP2 can comprise the nucleotide sequence set forth in SEQ ID NO:57 or the amino acid sequence set forth in SEQ ID NO:58. In an aspect, a disclosed TRD of MeCP2 can have a sequence having at least 75%, at least 80%, at least 85%, at least 90%, or at least 95% identity to the sequence set forth in SEQ ID NO:57 or SEQ ID NO:58 or a fragment thereof. In an aspect, a disclosed KRAB-MeCP2 can comprise the nucleotide sequence set forth in SEQ ID NO:62 or the amino acid sequence set forth in SEQ ID NO:63. In an aspect, a disclosed KRAB-MeCP2 can have a sequence having at least 75%, at least 80%, at least 85%, at least 90%, or at least 95% identity to the sequence set forth in SEQ ID NO:62 or SEQ ID NO:63 or a fragment thereof.

In an aspect, a disclosed gRNA can be designed to target exon 4 of the APOE gene or designed to target a protospacer-adjacent motif (PAM) created by a SNP rs429358 in exon 4 of the APOE gene. In an aspect, a disclosed gRNA can have the sequence set forth in any one of SEQ ID NO:05-SEQ ID NO:14, SEQ ID NO:25-SEQ ID NO:28, SEQ ID NO:39-SEQ ID NO:42, and SEQ ID NO:51-SEQ ID NO:52.

In an aspect, a disclosed Cas endonuclease can be fused to a disclosed polypeptide having an enzymatic activity. In an aspect, a disclosed Cas endonuclease can be dCas9 and the polypeptide can be DNMT3A. In an aspect, a dCas9-DNMT3A fusion protein can have the amino acid sequence set forth SEQ ID NO:19. In an aspect, a dCas9-DNMT3A fusion protein can be encoded by the sequence set forth in SEQ ID NO:20. In an aspect, a disclosed Cas endonuclease can be dVRER and the polypeptide can be DNMT3A. In an aspect, a disclosed dVRER-DNMT3A fusion protein can have the amino acid sequence set forth SEQ ID NO:38. In an aspect, a disclosed dVRER-DNMT3A fusion protein can be encoded by the sequence set forth in SEQ ID NO:37. In an aspect, a disclosed dVRER-DNMT3A fusion protein can have a sequence having at least 75%, at least 80%, at least 85%, at least 90%, or at least 95% identity to the sequence set forth in SEQ ID NO:38 or SEQ ID NO:37 or a fragment thereof.

In an aspect, a disclosed Cas endonuclease can be dCas9 and the polypeptide can be Krüppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed Cas endonuclease can be dVRER and the polypeptide can be Krüppel-associated box (KRAB), the transcription repression domain (TRD) of Methyl-CpG Binding Protein 2 (MeCP2), or a fusion of KRAB-MeCP2 (KRAB-MeCP2).

In an aspect of a disclosed method, a subject can be a human. In an aspect, a subject can be suspected of having or can be diagnosed with having Alzheimer's disease (such as, for example, LOAD). In an aspect, a disclosed subject can be symptomatic or asymptomatic. In an aspect, a disclosed method can comprise reducing the pathological phenotype associated with Alzheimer's disease. In an aspect, reducing the pathological phenotype associated with Alzheimer's disease can comprise reducing the A042/40 ratio and reducing the level of Tau. In an aspect, a disclosed method can comprise diagnosing the subject with Alzheimer's disease.

In an aspect, a disclosed method can comprise repeating one or more steps of the method and/or modifying one or more steps of the method.

In an aspect of a disclosed method, administering a disclosed vector can comprise intravenous administration, intracerebral administration, intra-CSF administration, intracerebroventricular (ICV) administration, intraventricular administration, intra-cistema magna (ICM) administration, intraparenchymal administration, intrathecal (lumbar, cistemal, or both) administration, or any combination thereof.

In an aspect, a disclosed method can comprise administering to the subject a therapeutically effective amount of a therapeutic agent, an effective amount of an immune modulator, or a combination thereof.

### G. Kits

Disclosed herein is a kit comprising one or more disclosed isolated nucleic acid molecules, disclosed vectors, disclosed lentiviral vectors, disclosed pharmaceutical formulations, disclosed host cells, disclosed guide RNAs, disclosed plasmids, or any combination thereof with or without additional therapeutic agents to treat, prevent, inhibit, and/or ameliorate one or more symptoms or complications associated AD or LOAD. In an aspect, a disclosed kit can be used in a disclosed method to reduce expression and/or activity of APOE regardless of the subject's genotype.

In an aspect, a disclosed kit can comprise at least two components constituting the kit. Together, the components constitute a functional unit for a given purpose (such as, for example, treating a subject diagnosed with or suspected of having A or LOAD). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed pharmaceutical formulation, a disclosed therapeutic agent, a disclosed reagent, or a combination thereof, and a label or package insert with instructions for use. In an aspect, suitable containers include, for example, bottles, vials, syringes, blister pack, etc. The containers can be formed from a variety of materials such as glass or plastic. The container can hold, for example, a disclosed pharmaceutical formulation and/or a disclosed therapeutic agent and can have a sterile access port (for example the container may be an intravenous solution bag or a vial having a stopper pierceable by a hypodermic injection needle). The label or package insert can indicate that a disclosed pharmaceutical formulation and/or a disclosed therapeutic agent can be used for treating, preventing, inhibiting, and/or ameliorating Alzheimer's disease (such as, for example, LOAD) or complications and/or symptoms associated with Alzheimer's disease. In an aspect, a disclosed kit can comprise additional components necessary for administration such as, for example, other buffers, diluents, filters, needles, and syringes.

### VI. EXAMPLES

Apolipoprotein E (ApoE) is encoded by the APOE gene (SEQ ID NO:01) positioned on chromosome 19q13.32 (GRCh 38: chr19:44,905,795-44,909,392). Two common coding SNPs in exon 4 of the gene give rise to three allelic variants, APOEe2 (SEQ ID NO:02), APOEe3 (SEQ ID NO:03), and APOEe4 (SEQ ID NO:04), encoding three corresponding protein isoforms that differ at two amino acid positions 112 and 158. The e4 allele of the apolipoprotein E gene (APOE e4) is the first, strongest, and most firmly established genetic risk factor for LOAD (Corder E H, et al. (1993) Science 261:921-923; Liu N, et al. (2008) Adv Genet. 60:335-405; Schmechel D E, et al. (1993) Proc Natl Acad Sci USA. 90:9649-9653; Saunders A M, et al. (1993) Neurology. 43:1467-1472). The initial discovery was made nearly 30 years ago by linkage analysis of pedigrees (Corder et al., 1993) and over the ensuing years it has become the most highly replicated genetic risk factor for LOAD (Corder et al., 1993; Liu et al. 2008; Schmechel et al., 1993; Saunders et al., 1993). Subsequent LOAD genome-wide association studies (GWAS) have confirmed strong associations with the APOE genomic region, and no other LOAD-association remotely approached the same level of significance (Harold D, et al. (2009) Nat Genet. 41:1088-1093; Lambert J C, et al. (2009) Nat Genet. 41:1094-1090; Heinzen E L, et al. (2009) J Alzheimers Dis. 19(1):69-77; Kamboh M I, et al. (2012) Mol Psychiatry. 17:1340-1346; Kamboh M I, et al. (2012) Transl Psychiatry. 2:e117; Seshadri, S. et al. (2010) JAMA. 303:1832-1840; Kunkle B W, et al. (2019) Nat Genet. 51:414-430; Lambert J C, et al. (2013) Nat Genet. 45:1452-1458; Coon K D, et al. (2007) J Clin Psychiatry. 68:613-618).

Carrying the APOE e4 variant significantly increases the lifetime risk for LOAD, whereas the number of e4 copies affects the level of risk and is associated with lower age of clinical disease onset (Corder et al., 1993; Farrer L A, et al. (1997) JAMA. 278:1349-1356), while APOE e3 is natural and APOE e2 conferred a protective effect (Saunders A M, et al. (1993) Neurology. 43:1467-1472; Reiman E M, et al. (2020) Nat Commun. 11:667). 40-65% of LOAD patients carry the e4 allele compared to 10-15% in the general population. Although, the precise molecular mechanisms underlying ApoE e4-mediated risk effects have not been fully elucidated, it was suggested that ApoE e4 acquired hyperfunction (gain of toxic effects) (Gottschalk W K, et al. (2016) J Alzheimers Dis Parkinsonism. 6(1):209) and increasing data indicate several cellular pathways through which ApoE e4 may exert toxicity associated with LOAD pathologic phenotypes (Huang Y A, et al. (2017) Cell. 168:427-441 e21; Sen A, et al. (2015) J Neurosci. 35:7538-7551; Theendakara V, et al. (2013) Proc Natl Acad Sci USA. 110:18303-18308; Theendakara V, et al. (2016) J Neurosci. 36:685-700; Min S W, et al. (2010) Neuron. 67:953-966; Tambini M D, et al. (2016) EMBO Rep. 17:27-36; Hatters D M, et al. (2006) J Mol Biol. 361:932-944). Collectively, these studies provide strong support to the concept that decreasing the levels of ApoE e4 specifically will have a therapeutic implication.

However, ApoE e4 as a target for LOAD remains significantly understudied, despite the few recent studies that have begun to pave the way. Another study utilized an antibody that specifically recognized the ApoE4 isoform and showed inhibition of A3 accumulation in the hippocampus and reversed the cognitive impairments compared to the control APOE4 mice (Luz I, et al. (2016) Curr Alzheimer Res. 13:918-929). Additional study applied the anti-human ApoE4 antibody and also found a reduction in A3 pathology characteristic of the APPS1-21/humanAPOE4 mice (Liao F, et al. (2018) J Clin Invest. 128:2144-2155). Collectively, these observations (Yang A, et al. (2021) Int J Mol Sci. 22(3):1244) demonstrated the beneficial effects of reducing the expression levels of ApoE, thus, supporting APOE as a promising therapeutics target for LOAD.

Moreover, accumulating evidence indicates that the increased overall expression of APOE plays an important role in the etiology of LOAD. Significant higher levels of APOE-mRNA in brain tissues obtained from e3/3 LOAD patients compared to 3/3 healthy donors, consistently with other reports showing elevated levels of APOE-mRNA in LOAD brains (Linnertz C, et al. (2014) Alzheimers Dement. 10:541-551; Zarow C, et al. (1998) Exp. Neurol. 149:79-86; Matsui T, et al. (2007) Brain Res. 1161:116-123; Akram A, et al. (2012) Neurobiol. Aging. 33:628e1). Further, studies using the APP/PS1 transgenic mice showed that lowering the ApoE protein levels ameliorated cognitive dysfunctions and AR pathology (Huynh T V, et al. (2017) Neuron. 96:1013-1023.e1014: Zheng J Y, et al. (2017) Neurobiol. Aging 2017, 54:112-132) independent of the APOE allele (Bien-Ly N, et al. (2012) J. Neurosci. 32:4803-4811; Kim J, et al. (2011) J. Neurosci. 31:18007-18012). While ApoE4 has received much attention for its LOAD-risk effect, there are clear changes in APOE expression associated with LOAD and independent of the e4 allele.

This means that the regulation of APOE expression can impact the risk to develop LOAD, making the modulation of the overall ApoE protein levels useful as a therapeutic target. To this end, reduction in APOE expression had beneficial effects, specifically a decrease in AR pathology. Antisense oligonucleotide (ASO) treatment lowered the APOE-mRNA and protein levels in the brains of APP/PS1-21 mouse model by at least 50%, leading to a significant decrease in AR pathology (Huynh T V, et al. (2017) Neuron 96:1013-1023 e4). Administration of anti-ApoE antibody have consistent effects on reducing A3 pathology and improved brain function and cognitive abilities (Kim J, et al. (2012) Annu Rev Neurosci. 31:75-93; Liao F, et al. (2014) J Neurosci. 34:7281-7292)).

Thus, as described below, methods of modifying the e4 isoform and reducing APOE expression and/or activity levels provide a promising avenue towards precision medicine in AD and especially LOAD.

### Example 1

Determining the Effect of APOE Genotypes of APOE-mRNA Levels

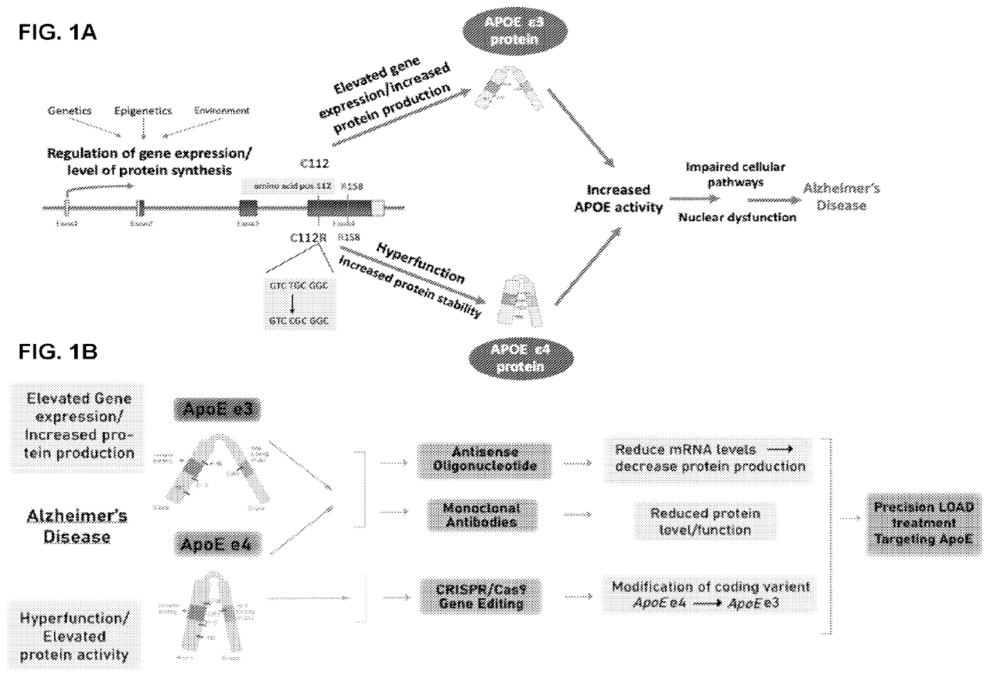

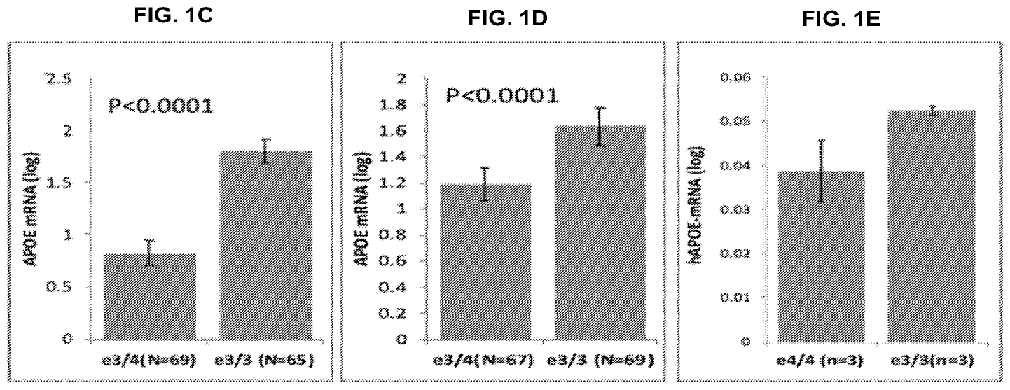

FIG. 1A shows a schematic model describing the mechanisms that lead to increased ApoE activity and by that mediate the pathogenic effect of APOE e4 and APOE e3 (differ in amino acid at position 112 Arg and Cys, respectively) on LOAD. FIG. 1B shows a diagram of the different technologies to target ApoE, including antisense oligonucleotide (ASO), monoclonal antibody (mAbs), and CRISPR/Cas9 gene editing technologies. Total RNA was extracted from brain samples (100 mg) using TRIzol reagent (Invitrogen, Carlsbad, CA) followed by purification with a RNeasy kit (Qiagen, Valencia, CA) according to the manufacturer's protocol. RNA concentration was determined spectrophotometrically at 260 nm, while the quality of the purified RNA was determined by 260 nm/280 nm ratio. All the RNA samples were of acceptable quality having ratios between 1.9 and 2.1. Sample quality and the absence of significant degradation products were confirmed by establishing that every sample had a RNA Integrity Number (RIN), as measured on an Agilent Bioanalyzer, of greater than 7. cDNA was synthesized using MultiScribe RT enzyme (Applied Biosystems, Foster City, CA) under these conditions: 10 min at 25° C. and 120 min at 37° C.

Real-time PCR was then used to quantify the levels of human TOMM40 mRNA and APOE mRNA. Duplicates of each sample were assayed by relative quantitative real-time PCR using the ABI 7900HT to determine the level of TOMM40 and APOE messages relative to the mRNAs for the housekeeping genes glyceraldehyde-3-phosphate dehydrogenase (GAPDH) and cyclophilin A (PPIA). ABI MGB probe and primer set assays were used to amplify APOE cDNA (ID Hs00171168_ml, 108 bp); and the two RNA reference controls, GAPDH (ID Hs99999905_ml, 122 bp) and PPIA (ID Hs99999904_ml, 98 bp) (Applied Biosystems, Foster City, CA). Each cDNA (10 ng) was amplified in duplicate in at least two independent runs (overall >4 repeats), using TaqMan Universal PCR master mix reagent (Applied Biosystems, Foster City, CA) and the following conditions: 2 min at 50° C., 10 min at 95° C., 40 cycles; 15 sec at 95° C.; 1 min at 60° C. As a negative control for the specificity of the amplification, RNA control samples that were not converted to cDNA (no-RT) and no-cDNA/RNA samples (no-template) were used in each plate. No amplification product was detected in control reactions. Data were analyzed with a threshold set in the linear range of amplification. The cycle number at which any particular sample crossed that threshold (Ct) was then used to determine fold difference, whereas the geometric mean of the two control genes served as a reference for normalization. Fold difference was calculated as 2^{-DDCt}; where DCt=[Ct(target)−Ct (reference)] and DDCt=[DCt(sample)]−[DCt(calibrator)].

The calibrator was a particular brain RNA sample used in every plate for normalization within and across runs. The variation of the DCt values among the calibrator replicates was less than 10%. For assay validation, standard curves for TOMAM40, APOE and each reference assay, GAPDH and PPIA using different amounts of human brain total RNA (0.1 ng-100 ng) were generated. In addition, the slope of the relative efficiency plot for TOMM40 and APOE with each internal control (GAPDH and PPIA) was determined to validate the assays. The slope in the relative efficiency plot for APOE and the reference genes were <0.1, showing a standard value required for the validation of the relative quantitative method. This methodology was published in Linnertz C, et al. (2014) Alzheimer's Dement. 10:541-551. FIG. 1C-FIG. 1E show the effect of APOE genotypes on APOE-mRNA levels. The fold levels of human APOE mRNA were assayed using qRT-PCR in temporal tissues (FIG. 1C) and in occipital tissues (FIG. 1D). FIG. 1E shows the level of human APOE-mRNA in whole brain tissues from humanized mice assayed by qRT-PCR.

### Example 2

### Schematic Representation of APOE Gene

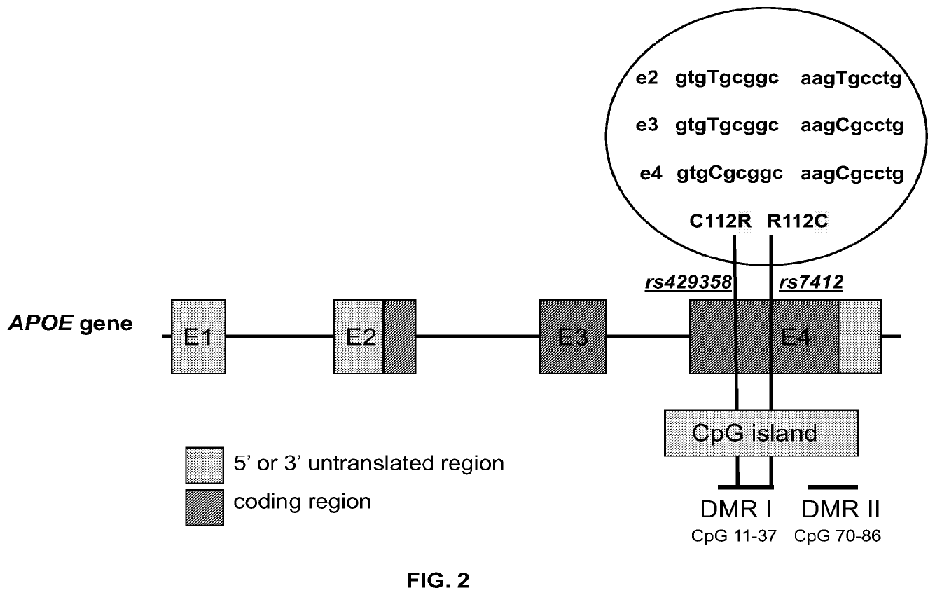

FIG. 2 shows a schematic representation of APOE gene, which is located at chromosome 19q13.2. The SNP rs429358 changes amino acid in position 112 and defines APOE e4 allele. The SNP rs7412 changes amino acid in position 158 and defines the APOE e2 allele. The CpG island in exon 4 is highlighted. DMRI and DMR2 regions are defined by two CGIs, which are marked in a yellow box. Exons 1-4 are designated in boxes. The translated exons are highlighted in dark blue. 5′-UTR and 3′-UTR of the gene are highlighted in light blue.

### Example 3

### Determining the DNA-Methylation Profile of the APOE Linkage Disequilibrium Region

The manufacturer's instructions were followed for the Infinium MethylationEPIC BeadChip Kit, which determined the profile of over 850,000 methylation sites quantitatively across the genome. Initial processing and quality control assessment of the methyl-array data were carried out using the minfi⁷² package from the R statistical programming environment (R Foundation for Statistical Computing, 2018). Normalization of the data was carried out separately within each of the three datasets using the ‘preprocessSWAN’ function to remove systematic differences across arrays. (Maksimovic J, et al. (2012) Genome Biol. 13:R44). Probes that have a detection p-value >0.05 in any sample were removed from subsequent analysis. A standard linear model was deployed for each analysis to identify differentially methylated probes. Probes were annotated with their genomic coordinates in the hg19 version of the human genome and the nearest gene to the probe was listed using the gene models provided by Ensembl (version 74).

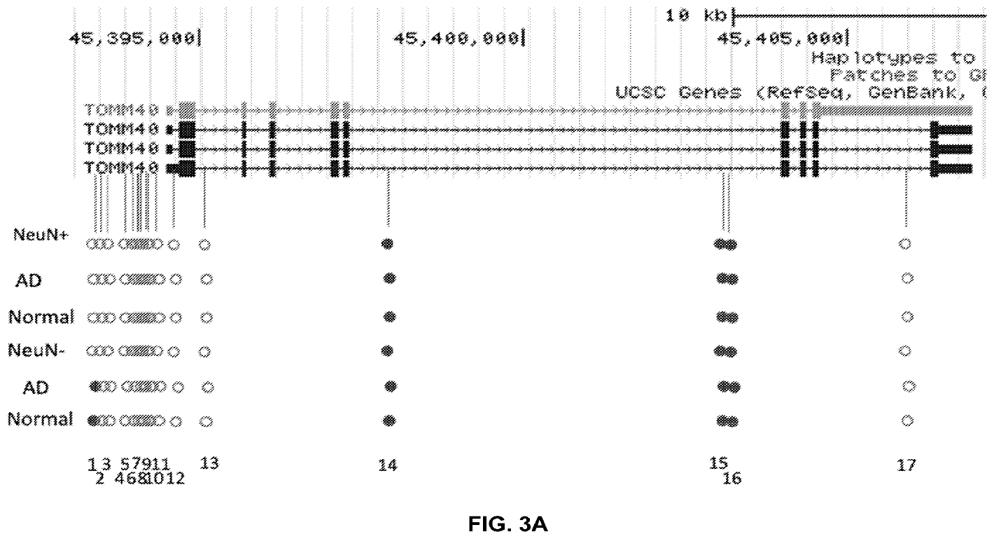

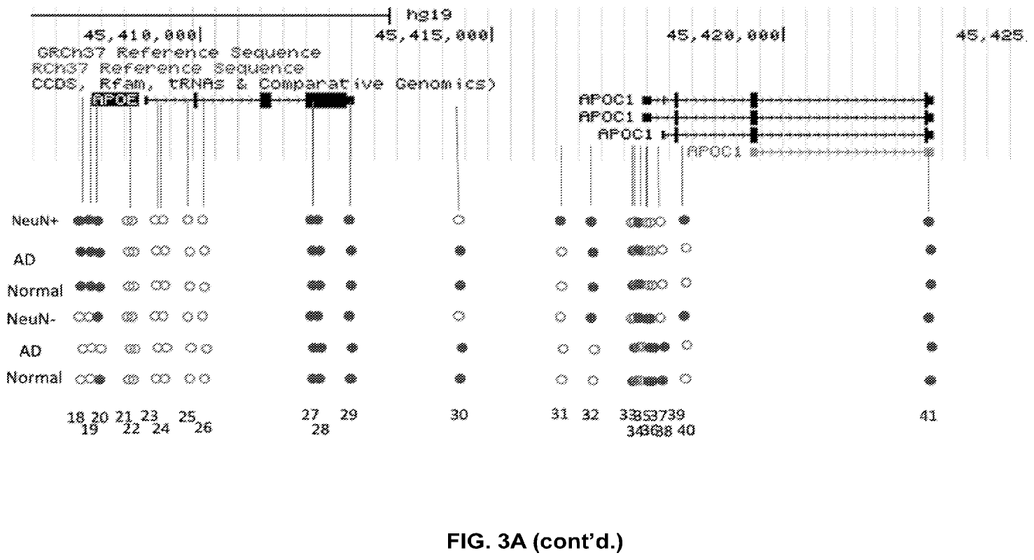

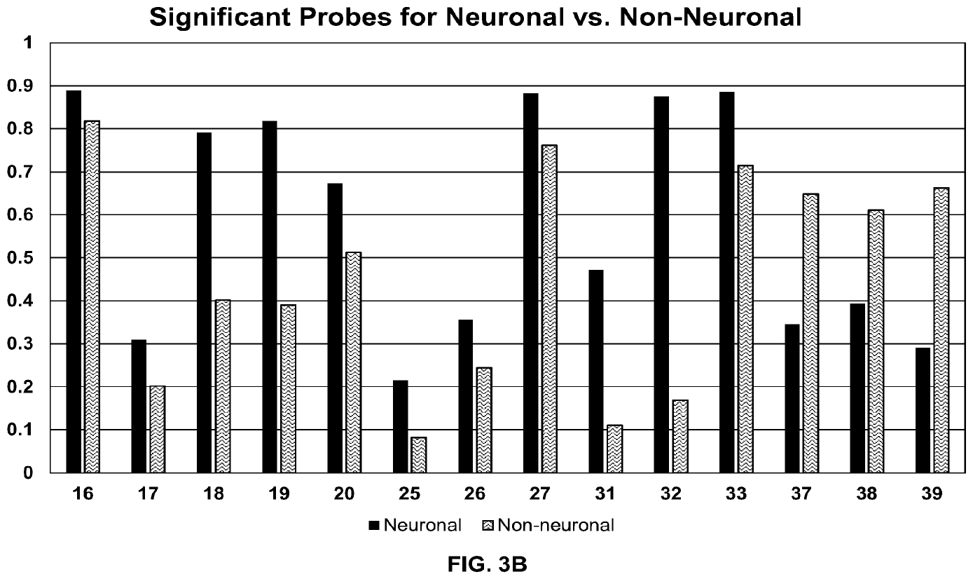

FIG. 3 shows the DNA-methylation profile of the APOE linkage disequilibrium (LD) region in FANS-sorted neuronal and non-neuronal nuclei. FIG. 3A shows a map of MethylEPIC array probes in the chr19: 45,393,000-45,424,000; hg19. The red circles represent probes with >0.5 methylation levels while the blue circles represent probes with <0.5 methylation levels. The APOE promoter region is hypomethylated and is an excellent target region for enhancement of DNA-methylation. FIG. 3B shows that probes showed significant differences in methylation levels between NeuN⁺ (n=16), NeuN⁻ sorted nuclei (n=16), or LOAD (n=8) vs. Normal (n=8). Solid bars represent neuronal population while the hatched bars represent the non-neuronal population. The accompanying table summarizes the p-values for each of the significant probes in FIG. 3B.

| Probe | p-value | Probe | p-value |
|---|---|---|---|
| 16 | 4.14E−11 | 31 | 9.67E−23 |
| 17 | 6.04E−15 | 32 | 1.88E−30 |
| 18 | 8.63E−15 | 33 | 2.24E−12 |
| 19 | 1.06E−16 | 37 | 1.09E−16 |
| 20 | 1.04E−09 | 38 | 8.72E−16 |
| 25 | 8.49E−26 | 39 | 9.21E−20 |
| 26 | 4.60E−10 | 11 | 0.000423 |
| 27 | 1.60E−08 | | |

### Example 4

### Targeting the Promoter Region of APOE Gene

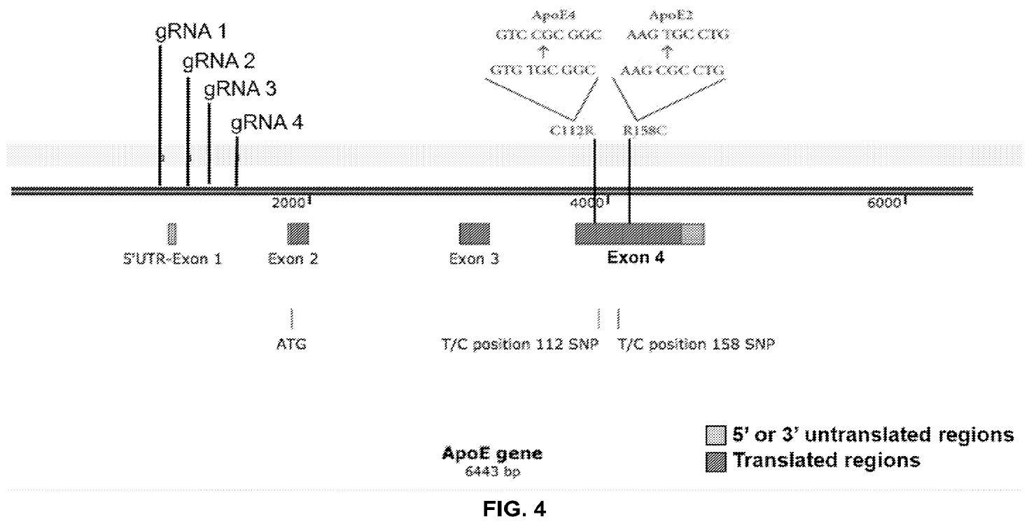

FIG. 4 shows the structure of human APOE gene and spCas9 gRNA design to target promoter region of the APOE gene. Genomic organization of the gene outlined in the lower panel while two SNPs within exon 4 are highlighted. The gRNA targeting promoter region of the gene is outlined. The 5′ UTR and 3′ UTR of the gene are indicated in boxes.

### Example 5

### Designing a Lentiviral Vector Carrying DNMT3A to Target the APOE Gene

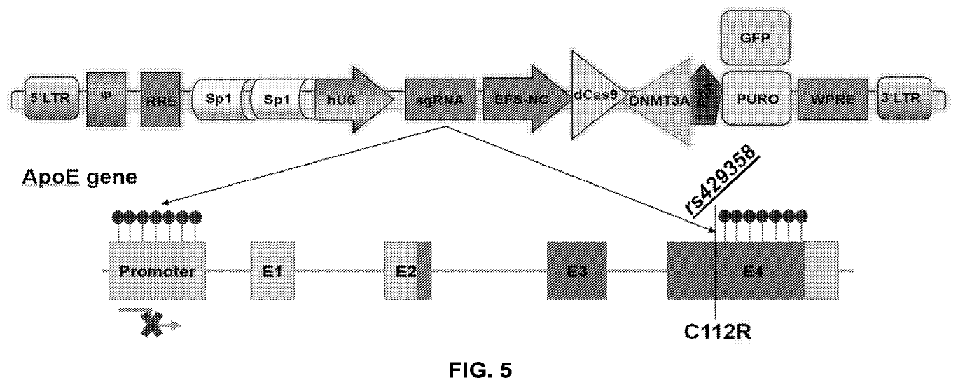

FIG. 5 shows the schematic representation of lentiviral vector system carrying DNMT3A to target the promoter and exon 4 regions of APOE gene. The 5′-LTR and the 3′-LTR represent long terminal repeats. Phi represents the packaging signal of the vector. RRE represents the rev responsive element responsible for binding REV protein of the virus. The Sp1 responsive element inclusion (Ortiniski et al., 2017; Kantor et al., 2018) demonstrated high production yield. The hU6 promoter drives expression of the gRNA and the EFS-NC promoter drives the expression of dCAS9 (to target promoter of APOE) or dVRER to target SNP (112) at the exon 4 region. The p2A signal separates the effector molecule from GFP/Puro reporters. WPRE is the Woodchuck Hepatitis Virus (WHP) Post-Transcriptional Regulatory Element (WPRE), which is a DNA sequence that when transcribed creates a tertiary structure enhancing expression. The arrow pointing to the promoter region highlights the binding of the dCas9-DNMT3A-gRNA to the promoter region or the SNP region that results in the DNA methylation (red lollipops) and downregulation of gene expression (represented with the red cross sign).

### Example 6

### Targeting the APOE Promoter Region with a gRNA-dCas9-DNMT3A Lentiviral Vector

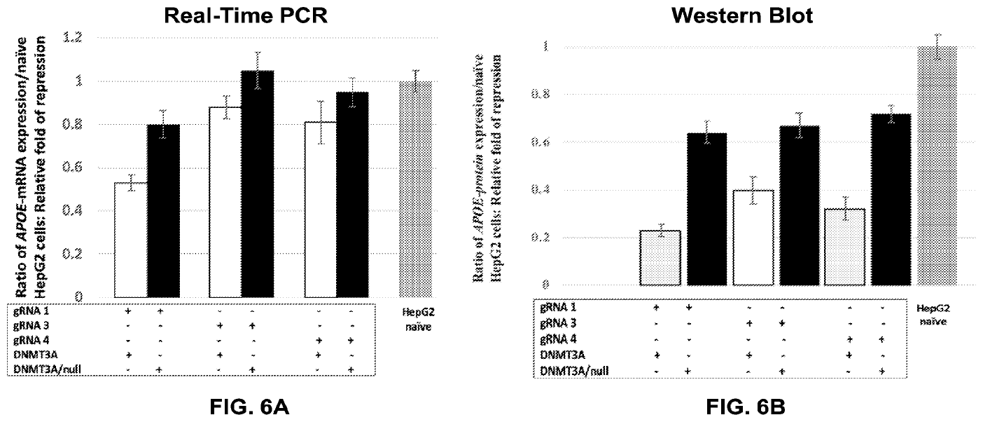

FIG. 6 shows the targeting of the promoter region of APOE with gRNA-dCas9-DNMT3A lentiviral vector system. Human hepatocytes HEPG2 cells were stably transduced with lentiviral vector carrying 4 different gRNA paired with dCas9-DNMT3A or dCAS9-DNMT3A null vectors. The table below shows the selection of gRNA to target APOE promoter region. The APOE promoter region was targeted by SpCas9-DNMT3A fusion protein via a set of gRNAs. Viral constructs 1026-1029 have an active version of DNMT3A while viral constructs 1030-1033 have an inactive version of DNMT3A (null). The sequences for the gRNAs targeting the promoter region of APOE for each construct are shown.

| Construct # | Guide | SEQ ID | | DNMT3A |
|---|---|---|---|---|
| (SEQ ID NO) | RNA | NO | Sequence | Activity |
| 1026 | gRNA1 | SEQ ID | gacaggggga | active |
| (SEQ ID | | NO: 25 | gccctataat | |
| NO: 29) | | | | |
| 1027 | gRNA2 | SEQ ID | tcaggagagc | active |
| (SEQ ID | | NO: 26 | tactcggggt | |
| NO: 30) | | | | |
| 1028 | gRNA3 | SEQ ID | actgggatgt | active |
| (SEQ ID | | NO: 27 | aagccatagc | |
| NO: 31) | | | | |
| 1029 | gRNA4 | SEQ ID | gttggagctt | active |
| (SEQ ID | | NO: 28 | agaatgtgaa | |
| NO: 32) | | | | |
| 1030 | gRNA1 | SEQ ID | gacaggggga | not active |
| (SEQ ID | | NO: 25 | gccctataat | |
| NO: 33) | | | | |
| 1031 | gRNA2 | SEQ ID | tcaggagagc | not active |
| (SEQ ID | | NO: 26 | tactcggggt | |
| NO: 34) | | | | |
| 1032 | gRNA3 | SEQ ID | actgggatgt | not active |
| (SEQ ID | | NO: 27 | aagccatagc | |
| NO: 35) | | | | |
| 1033 | gRNA4 | SEQ ID | gttggagctt | not active |
| SEQ ID | | NO: 28 | agaatgtgaa | |
| NO: 36) | | | | |

In FIG. 6A-FIG. 6B, the levels of the mRNA and protein downregulation were compared to untransduced naïve HEPG2 cells. The vectors delivering the active version of DNMT3A represented in white bars while the null mutants are shown in black bars. The experiments were repeated three time and the SD bars are highlighted. FIG. 6A shows the levels of RNA knockdown following the transduction with a lentiviral vector as assessed by real-time PCR (as described above). gRNA1 showed the most robust reduction in APOE-mRNA. FIG. 6B shows the levels of ApoE protein knockdown following the transduction with a lentivirval vector as assessed by western blot. The effects on the protein levels were comparable with the effects on the mRNA shown in FIG. 6A, demonstrating the most robust decrease in protein levels was driven by gRNA1. The levels of the mRNA and protein downregulation were compared to untransduced, naïve HEPG2 cells. In FIG. 6A-FIG. 6B, the vectors delivering the active version of DNMT3A were represented with white bars while null mutants were represented with black bars. The experiments were repeated three time and the SD bars are highlighted.

### Example 7

### Identifying the Location of VRER gRNAs and SpCas9 gRNAs Targeting the APOE Gene Promoter

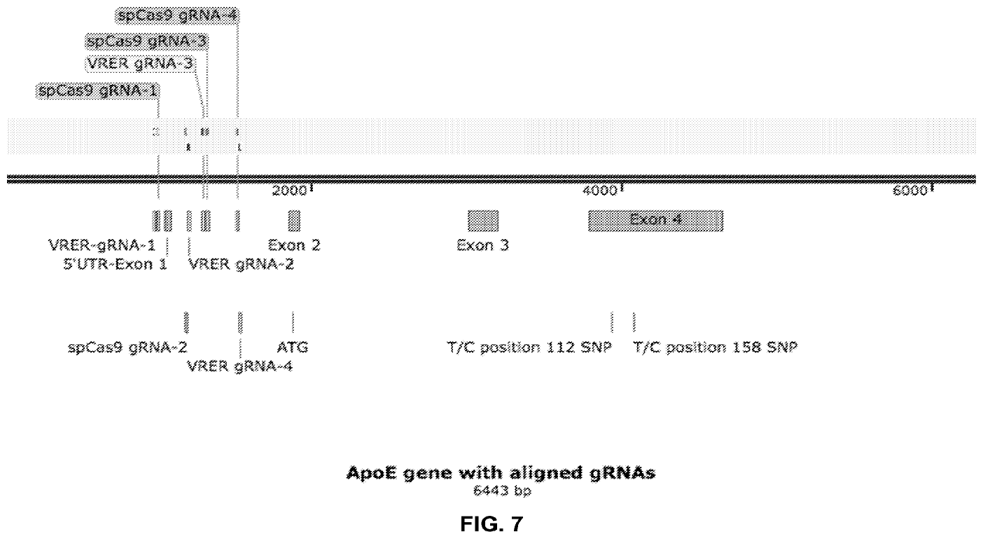

FIG. 7 shows the structure of human APOE gene and the position of the VRER gRNAs relative to positions of the spCas9 gRNAs, all of which targeted the promoter region of the APOE gene. Genomic organization of the gene outlined in the lower panel highlighting the 2 SNPs within exon 4. gRNA targeting promoter region of the gene are outlined. spCas9 gRNAs (in green) and VRER gRNAs (in yellow) positions. The 5′-UTR and the 3′-UTR of the gene are indicated in boxes.

### Example 8

### Validating the VRER System Using GFP-Reporter Cells

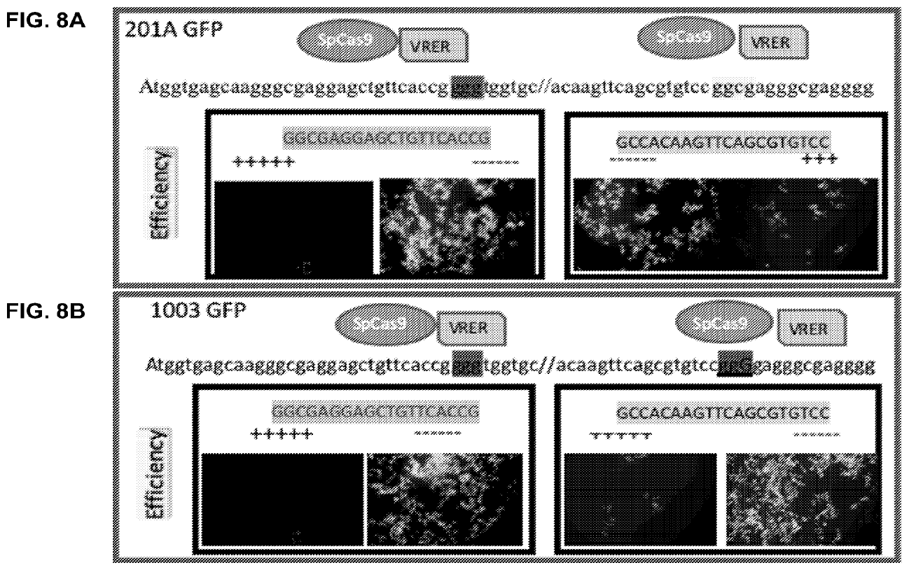

FIG. 8A-FIG. 8B show the validation of the VRER system using GFP-reporter cells. Here, an all-in-one lentiviral vector harboring catalytically active SpCas9 and VRER-Cas9 and gRNA targeting different regions of the eGFP was created. Two gRNAs targeting eGFP sequences adjunct to NGG or NGCG PAMs were selected. In FIG. 8, the two gRNA targeting GFP-ORF are highlighted in green (SEQ ID NO: 13-ggcgaggagctgttcaccg) and light-blue (SEQ ID NO:14-gccacaagttcagcgtgtcc). The NGG motif recognized by dCas9 is highlighted in pink. The NGCG motif recognized by VRER protein is highlighted in yellow. A GFP-reporter 293T cell line was created by stable transduction using lentiviral vector. The HEK293T cell lines expressed the WT version of GFP and the mutated version (C-to-G) are 201A GFP (FIG. 8A) and 1003 (FIG. 8B). GFP was subjected to site-directed mutagenesis to change the PAM motif for VRER enzyme NGCG to GGG, which is recognized by SpCas9. To preserve the amino acid composition, all modifications were made at the third-base positions. The target cells were transduced with SpCas9-gRNA-to-GFP vector VRER-gRNA-to-GFP vector to assess the specificity and efficacy of the corresponding enzymes. The efficiency and the specificity of the Cas9 and VRER toward NGG and NGCG PAMs was assessed by measuring GFP-depletion in the cells transduced with the respective viruses. This was recorded with a +/− score with +++++ (i.e., 5 “+”) having the maximal cleavage activity while −−−−− (i.e., 5 “−”) indicated minimal cleavage activity. The methods described in Ortinski P I, et al. (2017) Mol Ther Methods Clin Dev. 5:153-164, are incorporated by reference in its entirety for teaching of these methods. The specificity of VRER was found to be comparable to that of Cas9 while the efficacy was demonstrated to be significantly lower. In other words, VRER-dCas9 was capable of efficiently discriminating between NGG and NGCG PAM motifs. No detectable cleavage of the enzyme was observed in the context of NGG.

### Example 9

### Targeting the APOE Promoter Region with a dVRER-DNMT3A Lentiviral Vector

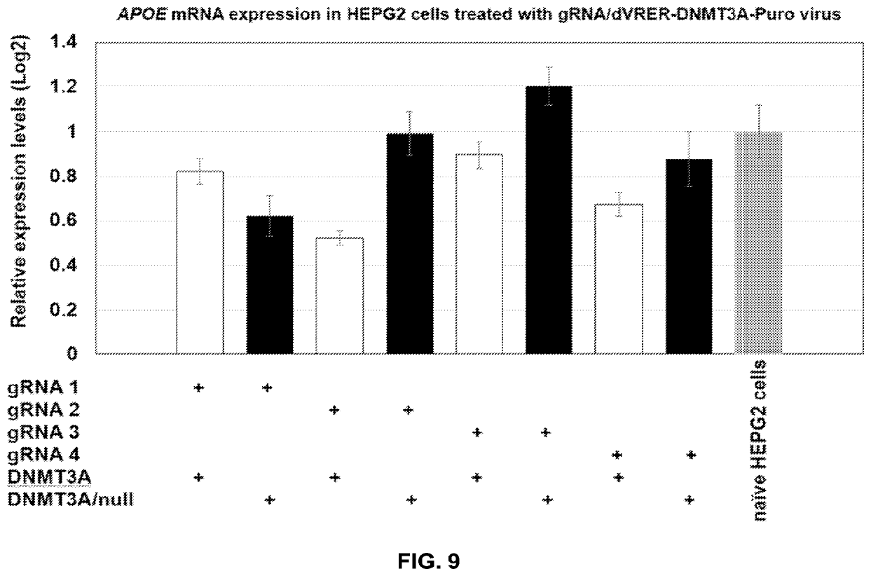

FIG. 9 shows the effect of targeting the promoter region of APOE with a gRNA-dVRER-DNMT3A lentiviral vector system. Human hepatocytes HEPG2 cells were stably transduced with lentiviral vector carrying 4 different gRNA targeting the APOE promoter region and paired with dVRER-DNMT3A or dVRER-DNMT3A null vectors. gRNA1 was gccctatccctgggggaggg (SEQ ID NO:39). gRNA2 was tcgggcttggggagaggagg (SEQ ID NO:40). gRNA3 was ctctccccaccccaccttct (SEQ ID NO:41). gRNA4 was tgtgaagggagaatgaggaa (SEQ ID NO:42). FIG. 9 shows the level of RNA knockdown following the transduction using real-time PCR. The levels of the mRNA downregulation was compared to untransduced, naïve HEPG2 cells. The vectors delivering the active version of DNMT3A are represented with white bars while the null mutants are represented with black bars. The experiments were repeated three times and the SD bars are highlighted. The gRNAs in this example are provided below.

| Construct # | Guide | gRNA Sequence | DNMT3A |
|---|---|---|---|
| SEQ ID NO | RNA | SEQ ID NO | Activity |
| 1105 | gRNA1 | gccctatccc | active |
| SEQ ID | | tgggggaggg | |
| NO: 43 | | SEQ ID | |
| | | NO: 39 | |
| 1106 | gRNA2 | tcgggcttgg | active |
| SEQ ID | | ggagaggagg | |
| NO: 44 | | SEQ ID | |
| | | NO: 40 | |
| 1107 | gRNA3 | ctctccccac | active |
| SEQ ID | | cccaccttct | |
| NO: 45 | | SEQ ID | |
| | | NO: 41 | |
| 1108 | gRNA4 | tgtgaaggga | active |
| SEQ ID | | gaatgaggaa | |
| NO: 46 | | SEQ ID | |
| | | NO: 42 | |
| 1109 | gRNA1 | gccctatccc | not active |
| SEQ ID | | tgggggaggg | |
| NO: 47 | | SEQ ID | |
| | | NO: 39 | |
| 1110 | gRNA2 | tcgggcttgg | not active |
| SEQ ID | | ggagaggagg | |
| NO: 48 | | SEQ ID | |
| | | NO: 40 | |
| 1111 | gRNA3 | ctctccccac | not active |
| SEQ ID | | cccaccttct | |
| NO: 49 | | SEQ ID | |
| | | NO: 41 | |
| 1112 | gRNA4 | tgtgaaggga | not active |
| SEQ ID | | gaatgaggaa | |
| NO: 50 | | SEQ ID | |
| | | NO: 42 | |

### Example 10

### Differentiating and Characterizing hiPSC-Derived Neurons

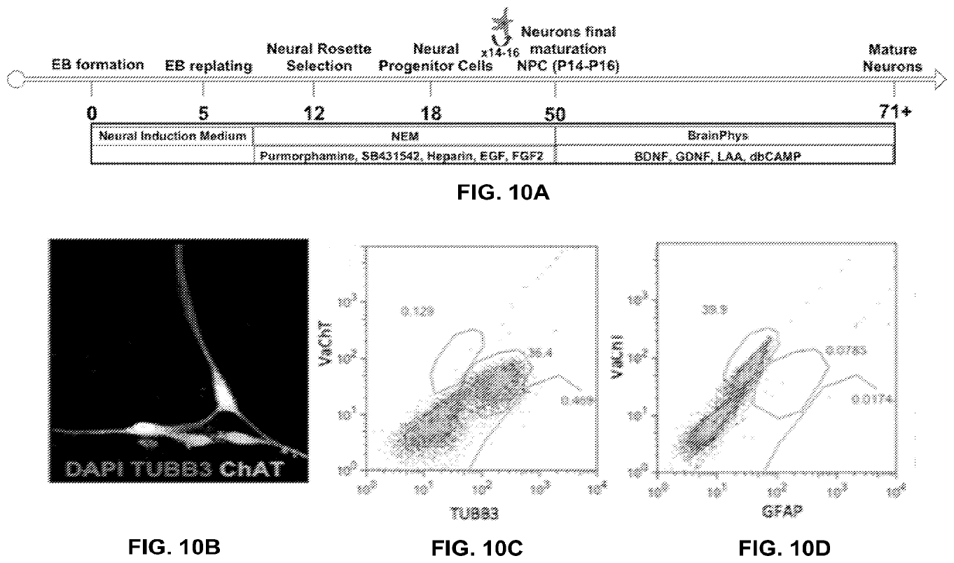

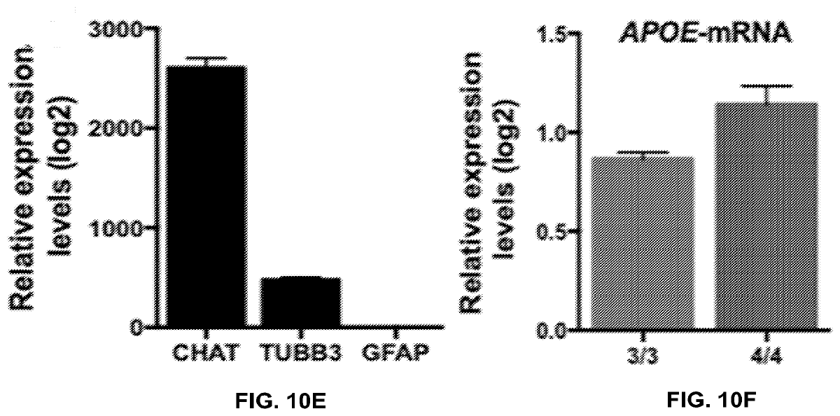

hiPSCs lines were differentiated into cholinergic neurons (the primary LOAD-affected neurons) as described by Tagliafierro L, et al. (2018) Hum Mol Genet. 28(3):407-421 and Tagliafierro L, et al. (2017) Alzheimer's Dement. 13(11):1237-1250. FIG. 10A shows the timeline of differentiation. FIG. 10B shows the representative immunocytochemistry of hiPSC-derived neurons. FACS-analysis shows co-expression of TUBB3 and VachT (36.4%) and absence of GFAP signal (FIG. 10C-FIG. 10D). FIG. 10E shows relative expression levels of neuronal-(TUBB3 and CHAT) and astrocytes (GFAP) specific markers; and FIG. 10F illustrate APOE-mRNA expression in isogenic APOE 3/3 and 4/4 hiPSC-derived neurons. APOE-mRNA expression in isogenic APOE 3/3 was greater than in isogenic APOE 4/4, which is consistent with the observation in human brain as shown in FIG. 10A-FIG. 10D.

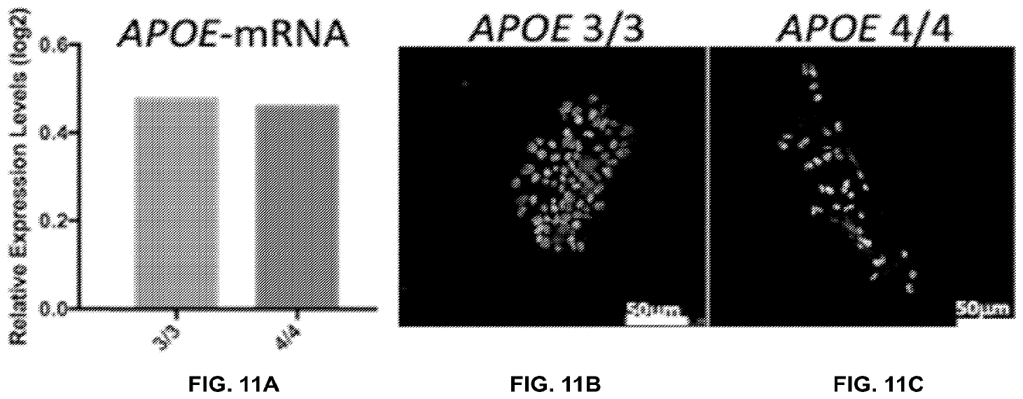

Furthermore, isogenic cell lines having APOE 3/3, APOE 3/4, and APOE 4/4 genotypes were cultured. Using CRISPR/Cas9 genome editing, isogenic lines were created such that the only difference is the SNP that defines the e4 allele. FIG. 11A-FIG. 11C show expression levels and immunohistochemical staining of isogenic APOE-hiPSC. FIG. 11A shows the fold levels of human APOE mRNA assayed by qRT-PCR using TaqMan assay. FIG. 11B (APOE 3/3) and FIG. 11C (APOE 4/4) show hiPSC shows cells stained with pluripotency markers OCT 4 and NANOG. (FROM GRANT)

### Example 11

### Evaluating Nuclear Envelope Markers in hiPSC-Derived Neurons

Loss of the integrity of the nuclear envelope has been associated with aging (Miller, et al., Cell Stem Cell 13, 691-705 (2013); Liu et al., Nature 491, 603-607 (2012)). To evaluate the nuclear architecture of the hiPSC-derived cells, nuclear envelope markers in isogenic APOE 3/3 and APOE 4/4 hiPSC-derived neurons were analyzed according to the methods described in Tagliafierro, et al., Hum Mol Genet (2018). In particular, the nuclear envelope integrity was assessed by using two specific nuclear envelope markers. First, Lamin A C (Miller, et al., Cell Stem Cell 13, 691-705 (2013); Tagliafierro, et al., Hum Mol Genet (2018)), wherein folded nuclei were counted as abnormal. Second, Lamin B1 (Liu et al., Nature 491, 603-607 (2012); Tagliafierro, et al., Hum Mol Genet (2018)), wherein nuclear circularity was quantified using the built-in ImageJ circularity plugin and assessed based on the Lamin B1 marker. 400 cells per staining were analyzed for two independent experiments.

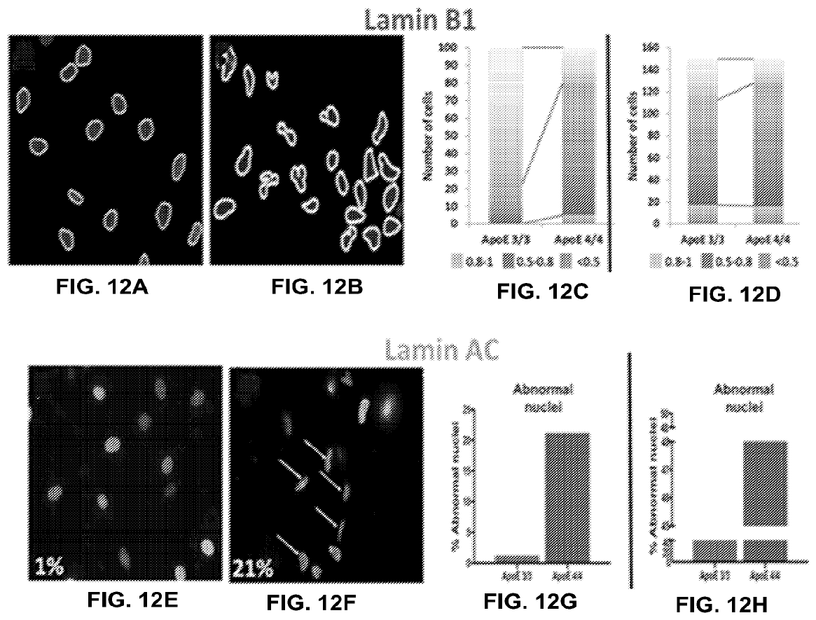

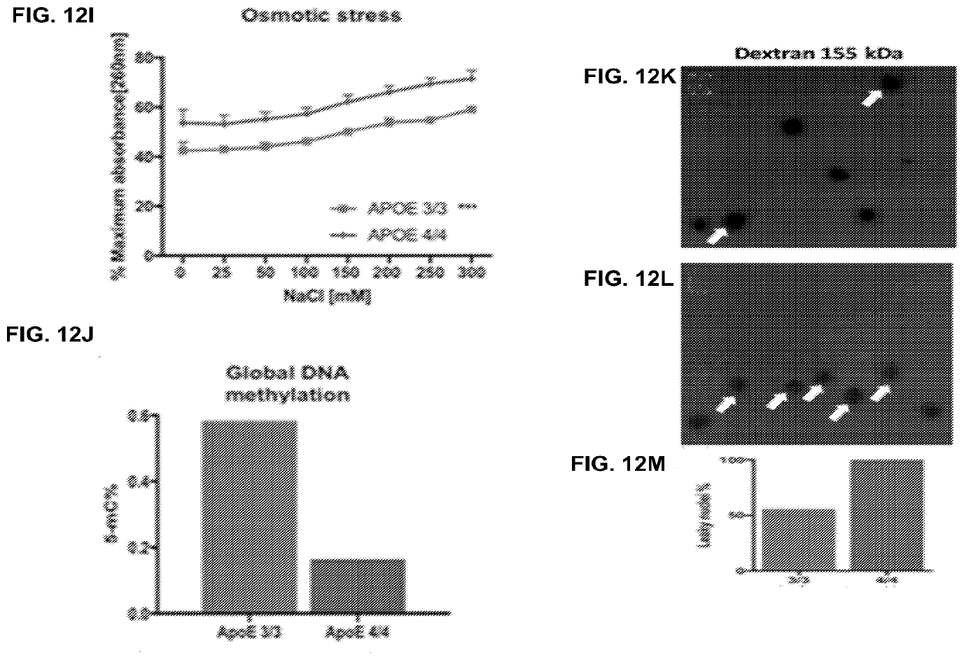

FIG. 12A-FIG. 12M show the results of the analysis of nuclear envelope markers in isogenic APOE 3/3 and APOE 4/4 hiPSC-derived neurons. FIG. 12A shows the immunocytochemistry for lamin B1 in APOE 3/3 hiPSC-derived neurons while FIG. 12B shows lamin B1 staining in APOE 4/4 hiPSC-derived neurons. As demonstrated in FIG. 12C, the quantification of the nuclear envelope circularity showed loss circularity in the APOE 4/4 hiPSC-derived neurons vs. the APOE 3/3 hiPSC-derived neurons before heat treatment while FIG. 12D shows the same comparison after heat treatment (i.e., heat-shock treatment as described by Vigouroux, et al., J. Cell Sci. 114, 4459-4468 (2001)). FIG. 12E shows the immunocytochemistry for lamin AC in APOE 3/3 hiPSC-derived neurons while FIG. 12F shows lamin B1 staining in APOE 4/4 hiPSC-derived neurons. FIG. 12G shows the proportion of cells with abnormal nuclear morphology in the APOE 4/4 hiPSC-derived neurons vs. the APOE 3/3 hiPSC-derived neurons before heat treatment while FIG. 12H shows the same comparison after heat treatment (described by Vigouroux et al., 2001).

As shown in FIG. 12I, osmotic stress was applied by incubating the cells with an increasing concentration of NaCl₂ (Czubryt, et al., Mol. Cell. Biochem. 172, 97-102 (1997)), which resulted in an increased sensitivity of the nuclear envelope in the APOE 4/4 neurons compared to the APOE 3/3 neurons. Using a commercially available kit, the percentage of the 5 methylcytosine (5 mC %) was measured (Jones M J, et al. (2015) Aging Cell. 14(6):924-932). FIG. 12J shows the decrease in global 5-mC % in APOE 4/4 hiPSC-derived neurons as compared to APOE 3/3 hiPSC-derived neurons.

For the nuclear dextran size exclusion assay (described by Eftekharzadeh B, et al. (2019) Neuron. 101(2):349; D'Angelo M A, et al. (2009) Cell. 136:284-295), low-molecular-weight (<25 kDa) dextran was expected to freely traverse nuclear pore complexes and fill the nucleus, whereas higher molecular weight (e.g., 70-kDa and 500-kDa) dextrans were expected to be excluded from the nucleoplasm when the nuclear pore complexes are intact. Nuclei were isolated with a sucrose gradient and incubated with fluorescent dextrans of different molecular weight. Intranuclear 155-kDa dextran indicated leakiness of the nuclear membrane. FIG. 12K and FIG. 12L shows the nuclear leakage as assessed by a dextran assay using 155 kDa fluorescently-label molecule APOE 3/3 hiPSC-derived neurons and 4/4 hiPSC-derived neurons, respectively. FIG. 12M shows the percentage of leaky nuclei for both APOE 3/3 and APO 4/4 hiPSC-derived neurons.

### Example 12

### Examining the Methylation Profile of the APOE Linkage Disequilibrium Region in hiPSC-Derived Neurons

Pyrosequencing was performed using bisulfite converted DNA to quantitatively determine the methylation levels according to the protocol set forth in Bassil C F, et al. (2013) Methods Mol Biol. 1049:95-107, which is incorporated by reference in its entirety for its teaching of a bisulfite pyrosequencing protocol. The assay was designed across APOE exon 4 covered CpG 11-38 (chr19: 45411858-45412063; hg19) as described by Tulloch J, et al. (2018) Brain Res. 1698:179-186, which is incorporated by reference in its entirety for its teaching of the CpG assay design.

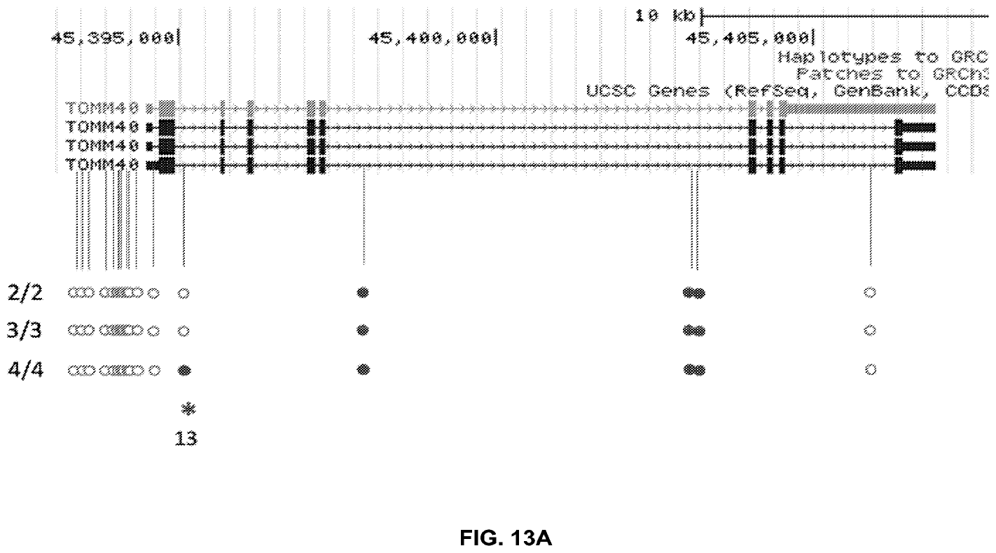

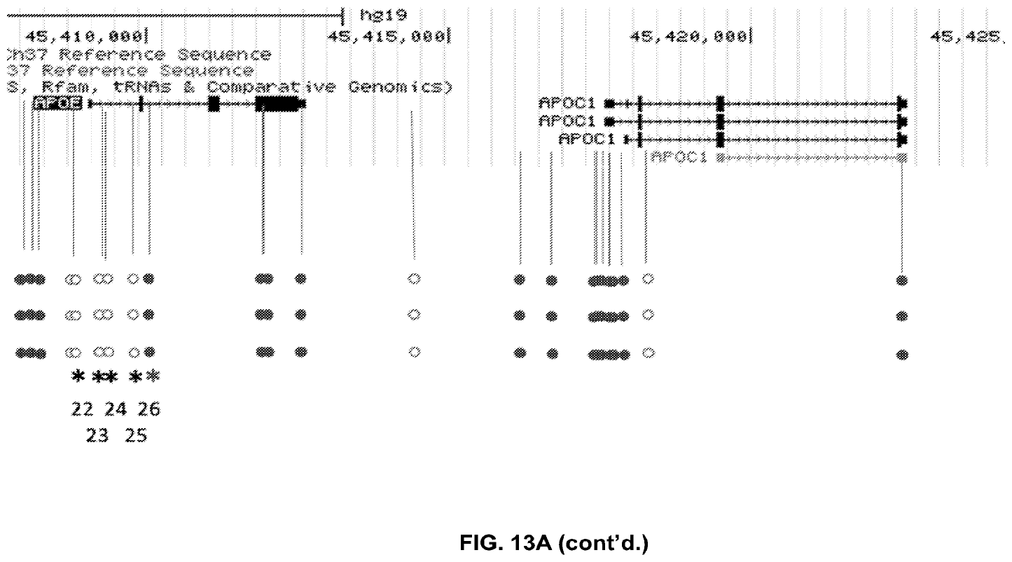

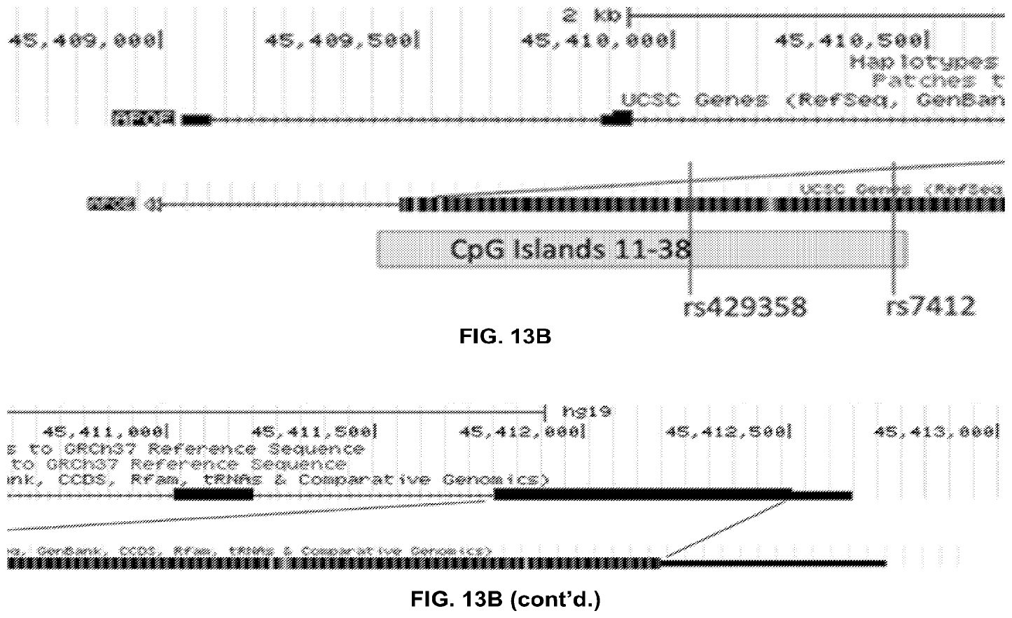

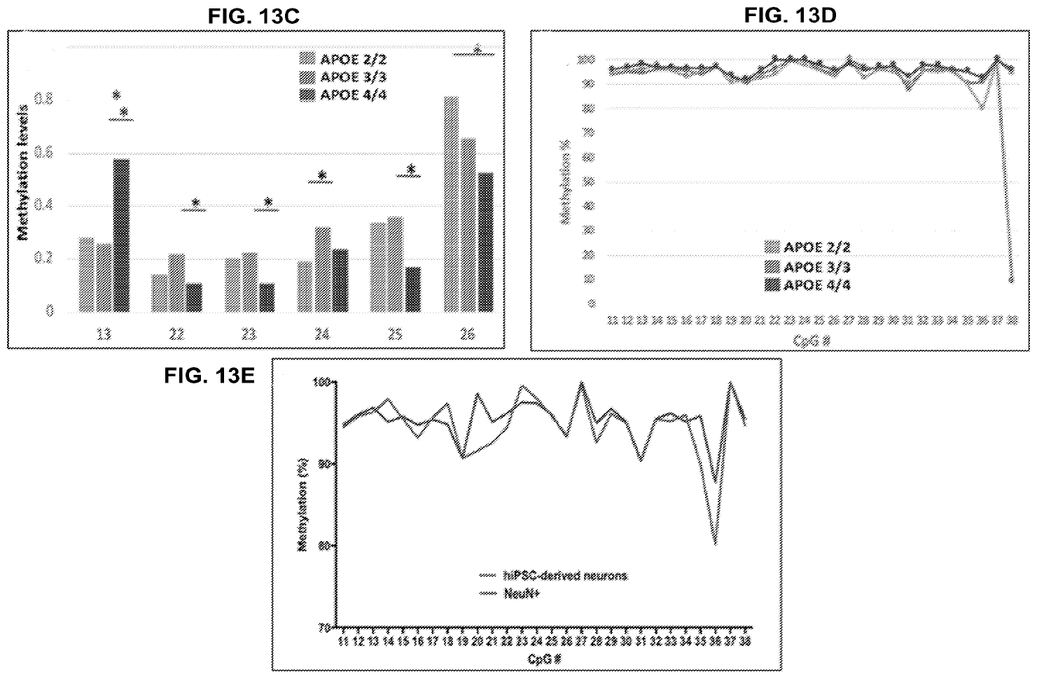

FIG. 13A-FIG. 13E shows the methylation profile of the APOE linkage disequilibrium (LD) region in isogenic APOE hiPSC-derived neurons. FIG. 13A shows a map of MethylEPIC array probes in chromosome 19 from 45,393,000-45,424,000 (hg19). Those probes with >0.5 methylation levels are highlighted in red. Those probes with <0.5 methylation levels are highlighted in blue. Significant differences in methylation between the APOE neuronal lines are shown using asterisks as follows: black asterisk (>0.1) and red asterisk (>0.2). Because the APOE promoter region was hypomethylated, it was an excellent target region for enhancement of DNA-methylation. FIG. 13B shows a schematic representation of the 27 CpG islands for pyrosequencing in the APOE region, i.e., chromosome 19 from 45,411,858-45,412,079 (hg19). FIG. 13C shows those probes that had significant differences in DNA-methylation levels between isogenic APOE hiPSC-derived neurons. FIG. 13D shows the methylation level (%) of the CpG 11-38 that was quantitatively determined in the isogenic hiPSC-derived neurons using pyrosequencing. FIG. 13E shows a comparison of the methylation level (%) of CpG 11-38 between hiPSC-derived neurons and NeuN⁺ FANS-sorted nuclei using pyrosequencing. Here, the DNA-methylation profiles of the hiPSC-derived neurons were comparable to those observed for the human brain sorted neuronal nuclei (indicating that the hiPSC-derived neuronal system was suitable for drug discovery studies aiming at DNA-methylation editing).

### Example 13

### Examining Alzheimer's Disease Phenotypes in hiPSC-Derived Neurons

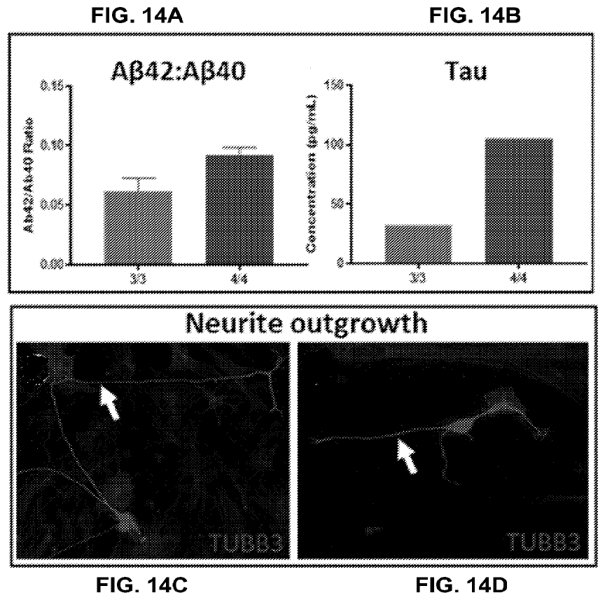

To generate the data presented in FIG. 14A, an ELISA kit and a V-PLEX Plus AR Peptide Panel 1 (6E10) Kit (Cat: K15200G-1) was used to measure secreted levels of Ab40 and Ab42. The Ab42/40 ratio was then calculated according to Lin Y T, et al. (2018) Neuron. 98(6):1294 and Wang C, et al. (2018) Nat Med. 24(5):647-657, both of which are incorporated by reference in their entirety for the teaching of these protocols. As shown in FIG. 14B, the total tau and pTau levels were measured by ELISA kits using (i) an Invitrogen Human Tau (Total) ELISA Kit (Cat: KHB0041) and (ii) an Invitrogen Human Tau [pT181] phosphoELISA™ ELISA Kit (Cat: KH00631). The neurite outgrowth in FIG. 14C and FIG. 14D was assessed by TUBB3 staining followed by a tracing analysis to determine (i) the number of neurites originating from the soma of each neuron, (ii) the individual length of the longest single neurite, and (iii) the total length of all neurites in a single neuron (Lin Y T, et al. (2018) and Wang C, et al. (2018)). FIG. 14A-FIG. 14D present the disease related cellular perturbations and pathological characteristics of the hiPSC-derived neuronal model system that are being used in the first stage for the in vitro studies.

### Example 14

### Examining DNA Methylation in the APOE Promoter Target Region

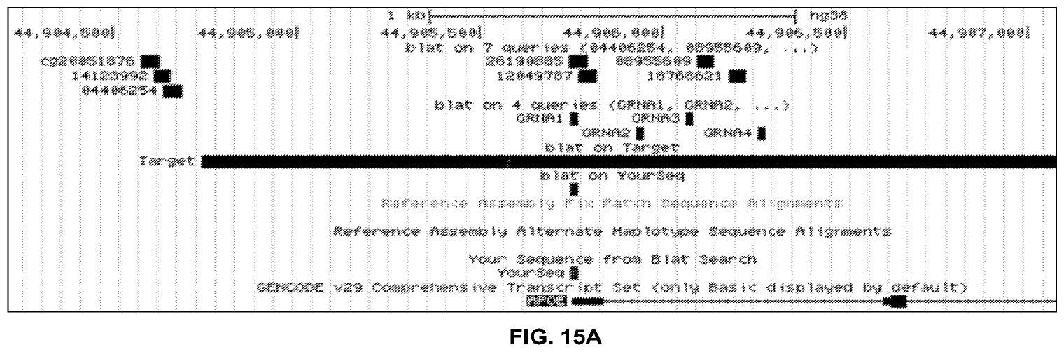

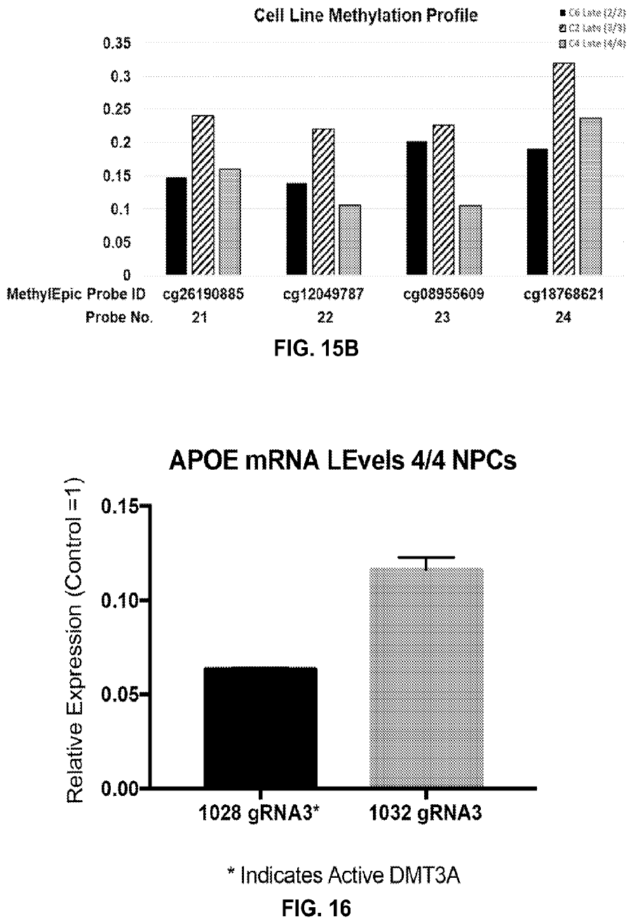

FIG. 15A shows a map of the targeted APOE promoter region was generated using a UCSC genome browser viewer. In the upper panel, black bars indicate the positions of the target region, the designed gRNAs, and the MethylEpic probes. In the lower panel, the APOE gene structure is shown with the promoter, exon 1, intron 1, and the TSS. FIG. 15B shows the analysis of DNA-methylation within the APOE-promoter target region, specifically those probes that overlapped the target region and showed differences in DNA-methylation levels between the isogenic APOE hiPSC-derived neurons. These lines will be used in the first stage for the in vitro studies for proof of concept of the developed epigenome-editing system as a therapeutic strategy for precision medicine in Alzheimer's.

### Example 15

### Targeting the APOE Promoter Region with a gRNA-dCas9-DNMT3A Lentiviral Vector

hiPSC-derived cholinergic neurons homozygote to the APOE e4 allele were stably transduced with lentiviral vector carrying gRNA3 paired with dCas9-DNMT3A or dCAS9-DNMT3A null vectors. FIG. 16 shows the levels of RNA knockdown following the transduction as assessed by real-time PCR.

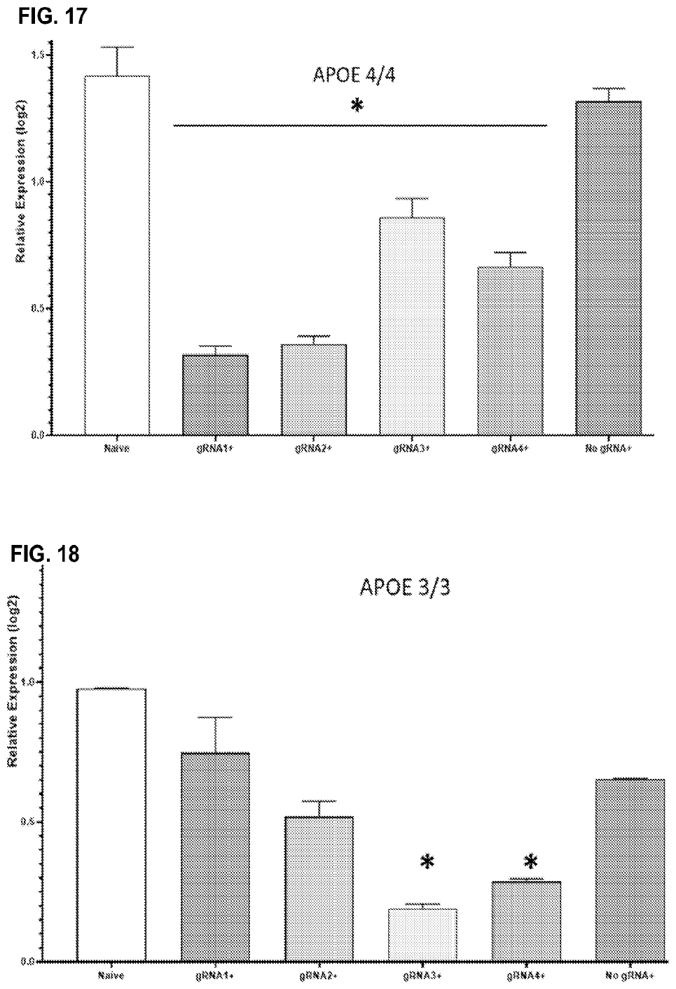

Similarly, hiPSC-derived cholinergic neurons homozygote to the APOE e4 allele were stably transduced with lentiviral vector carrying gRNAs 1-4 paired with dCas9-DNMT3A or with a dCAS9-DNMT3A vector with no-gRNA. FIG. 17 shows the level of mRNA knockdown following the transduction as assessed by real-time PCR. The vectors having a gRNA all significantly knocked down the level of APOE mRNA compared to either a null vector or a vector having no gRNA.

Then, hiPSC-derived cholinergic neurons homozygote to the APOE e3 allele were stably transduced with lentiviral vector carrying gRNAs 1-4 paired with dCas9-DNMT3A or a dCAS9-DNMT3A vector with no-gRNA. FIG. 18 shows the level of mRNA knockdown following the transduction as assessed by real-time PCR. The vectors having gRNA3 or gRNA4 significantly knocked down the level of APOE mRNA compared to either a null vector or a vector having gRNA1 or gRNA2.

### Example 16

### Targeting the APOE Promoter Region with a gRNA-dVRER-DNMT3A Lentiviral Vector

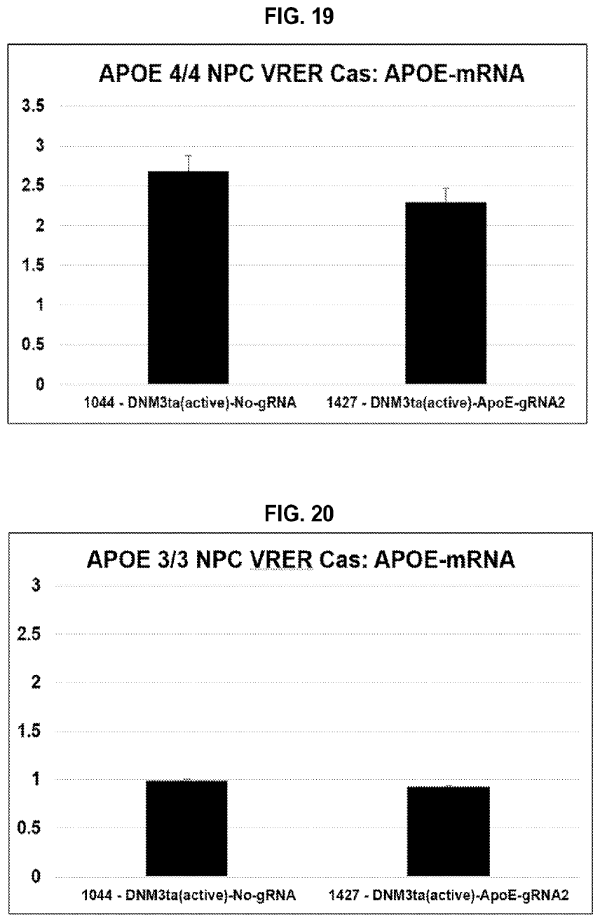

hiPSC-derived cholinergic neurons homozygote to the APOE e4 allele were stably transduced with a lentiviral vector carrying a gRNA that was 2′ paired with dVRER-DNMT3A or with a dVRER-DNMT3A lentiviral vector with no gRNA. FIG. 19 shows the level of mRNA knockdown following the transduction as assessed by real-time PCR assessed. The vector having gRNA2 achieved a 15% reduction in the level of APOE mRNA compared to the vector having no gRNA. Similarly, hiPSC-derived cholinergic neurons homozygote to the APOE e3 allele were stably transduced with a lentiviral vector carrying a gRNAs 2′ paired with dVRER-DNMT3A or a dVRER-DNMT3A lentiviral vector with no gRNA. FIG. 20 shows the level of mRNA knockdown following the transduction as assessed by real-time PCR assessed. No changes in the level of APOE mRNA were observed.

### Example 17

### Designing Lentiviral Vectors Having Additional Effector Molecules

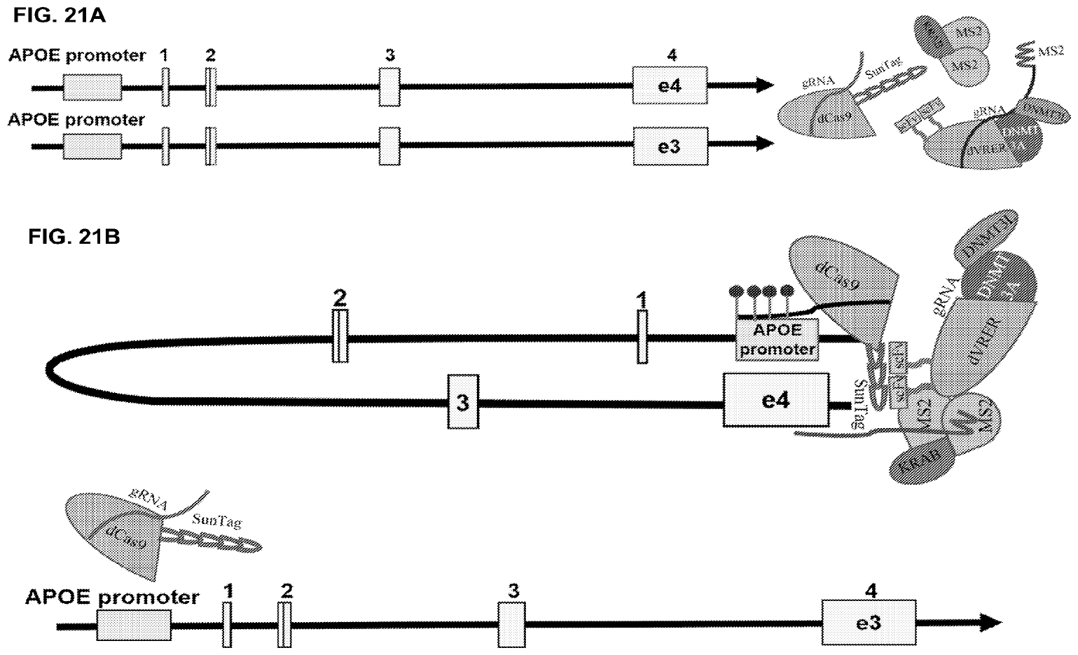

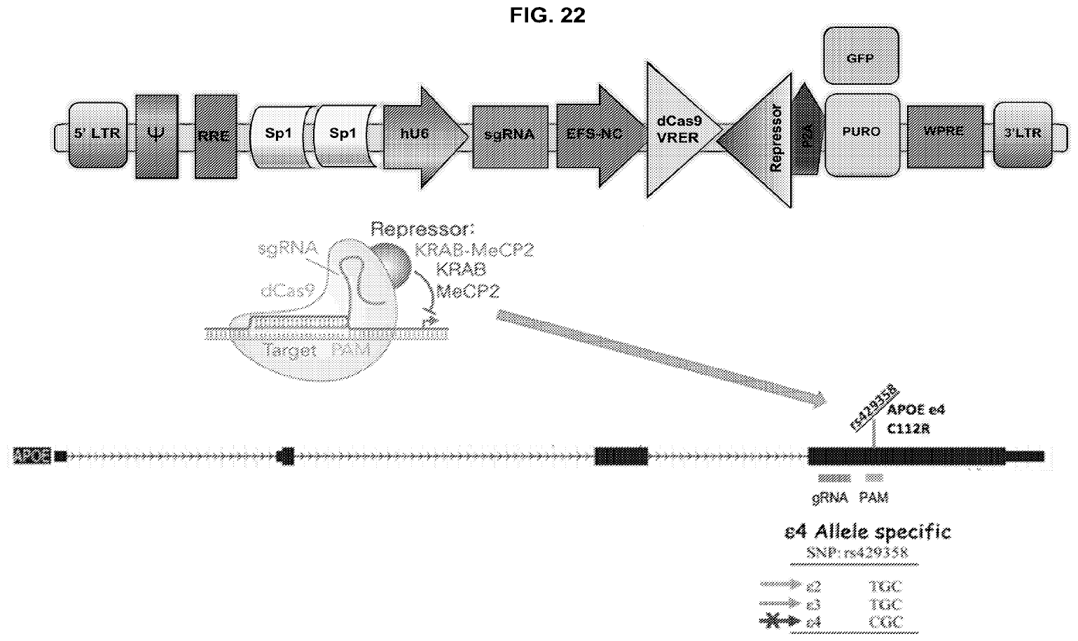

A strategy to silence APOEe4 allele using DNMT3A-DNMT3L enzymes and KRAB repressor as the effector molecules was developed. FIG. 21A shows a schematic representation of the APOE gene including promoter region and exon 1-4. Here, two lentiviral vector systems are established. The first lentiviral vector carries dCAS9-gRNA-to-promoter. The vector also harbors a SunTag epitope recognized by single-chain scFv protein. The second lentiviral vector carries dVRER and gRNA for specific targeting of SNP rs429358 in the exon 4 (on the e4) and DNMT3A-DNMT3L effectors. The gRNA with the MS2 binding sites allows for the recruitment of KRAB repressor via the MS2-protein (fusion).

FIG. 21B shows that following lentiviral vector-delivery, the dCAS9-gRNA-SunTag binds to the promoter region on both alleles. However, it is inactive on the e3-allele as it lacks the effector molecules. The recruitment of dVRER via specific binding mediated throughout the recognition of the PAM (NGCG) brings the effector molecules in the action. Following interaction between SunTag-scFv, DNA on the e4 is looped out and two the effector molecules, KRAB and DNMT3A-L, repress and methylate the promoter of the e4. The SunTag-MS2-bridging system allows specific repression of the e4-allele.

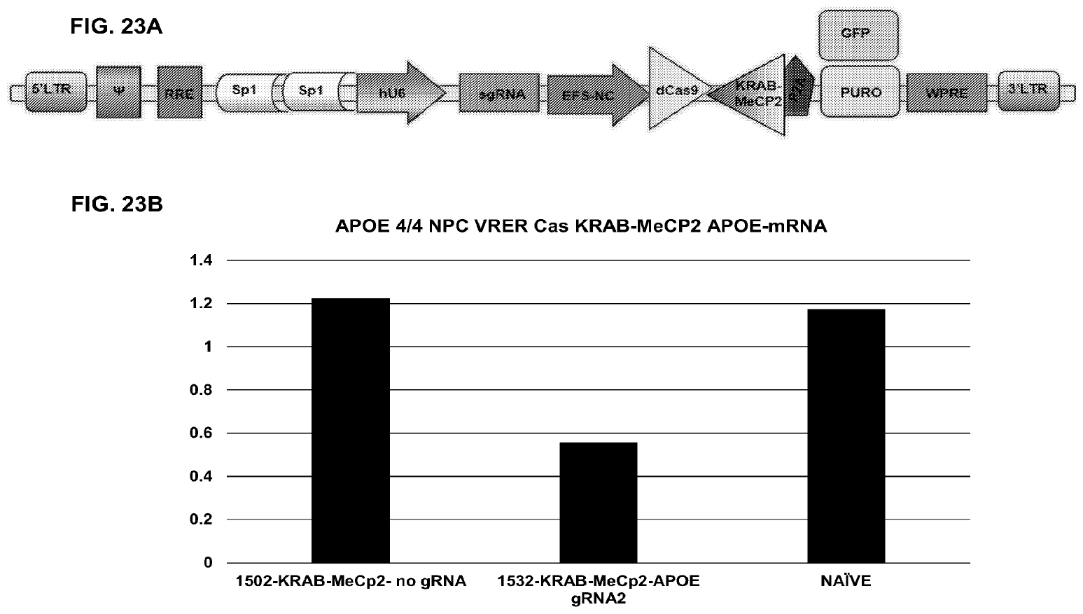

FIG. 22 shows a schematic illustration of the lentiviral vector carrying gRNA-dCas9/dVRER-repressor transgene. The vector backbone was optimized by inclosing Sp1 binding sites². dCas9-KRAB/MeCP2/KRAB-MeCP2 fusion expressed from EFS-NC promoter. Human U6 promoter drives gRNA expression. Other elements of the vector are highlighted^{2,3}. The vector carries gRNA to target regulatory element within exon 4 overlapping the e4-SNP, to specifically target the ApoE4 allele. The expected downregulation in the transcription activity of the different APOE alleles is denoted. FIG. 23A-FIG. 23B show the targeting exon 4 region of APOE with a gRNA-dVRER-DNMT3A lentiviral vector system. FIG. 23A shows that the construct was identical to that of FIG. 5 but for the addition of the repressor to the fused domains of KRAB-MeCP2. FIG. 23B shows the mRNA level in hiPSC-derived cholinergic neurons homozygote to the APOE e4 allele following stable transduction with lentiviral vector carrying a gRNA 2′-paired with dVRER-CRAB MeCp2 or a lentiviral vector carrying a dVRER-KRAB MeCp2 vector with no gRNA. Real-time PCR assessed the levels of mRNA knockdown following the transduction. The vector have a gRNA caused a >50% reduction in the level of APOE mRNA.

### SUMMARY OF EXAMPLES

The Examples provided herein show that epigenome-based therapy paired with lentiviral vector is an advantageous strategy for the treatment of LOAD because it has versatility, low immunogenicity, and remarkable suitability for viral-mediated gene transfers. Pre-existing approaches including antisense oligonucleotides (ASO) and immunotherapy are plagued by significant disadvantages such as low efficiency and specificity, low stability and solubility, adverse immunoreactivity, and inability to penetrate blood-brain barrier (BBB). Epigenome editing also holds key advantages over direct gene knockout because epigenome editing triggers the natural cellular system that leads to gene silencing by a defined mechanism (Rittiner J E, et al. (2020) Front Mol Neurosci. 13:148). By contrast, knocking out a gene by conventional genome editing depends on targeted DNA double-strand breakage followed by repair, which can occur via variable repair pathways that are not fully predictable.

The APOE-targeted epigenome therapy described herein combines emerging innovative genomic technologies and delivery techniques to overcome these limitations. As demonstrated by the data presented in the Examples, the allelic discrimination approach is innovative as it allows a precise and fine-tuned downregulation of APOEe4 allele expression. The utility of dCas9-variant, VRER (Kleinstiver B P, et al. (2015) Nature. 523:481-485) in gene therapy is innovative and the combination of the epigenomic modification approach and the strategy to target allele specific is novel. The novel vector system disclosed herein circumvents several challenges related to gene therapy. It has a high efficiency for delivery of oversized CRISPR/Cas9 components. It is suitable for a broad range of cellular tropisms. It has low cytotoxicity and immunogenicity. It generates long-term and sustainable expression of the transgene which will ensure that the epigenetic changes imprinted within APOE exon 4 are permanent. Importantly, lentiviruses are very efficient in transducing post-mitotic neurons in vivo.

### Sequences

In an aspect, a disclosed ApoE gene can comprise the sequence set forth below or a sequence having 70%, 75%, 80%, 85%, 90%, 95%, or more identity to the sequence set forth below:

(SEQ ID NO: 01)
ggaacttgatgctcagagaggacaagtcatttgcccaagg
tcacacagctggcaactggcagagccaggattcacgccct
ggcaatttgactccagaatcctaaccttaacccagaagca
cggcttcaagcccctggaaaccacaatacctgtggcagcc
agggggaggtgctggaatctcatttcacatgtggggaggg
ggctcccctgtgctcaaggtcacaaccaaagaggaagctg
tgattaaaacccaggtcccatttgcaaagcctcgactttt
agcaggtgcatcatactgttcccacccctcccatcccact
tctgtccagccgcctagccccactttcttttttttctttt
tttgagacagtctccctcttgctgaggctggagtgcagtg
gcgagatctcggctcactgtaacctccgcctcccgggttc
aagcgattctcctgcctcagcctcccaagtagctaggatt
acaggcgcccgccaccacgcctggctaacttttgtatttt
tagtagagatggggtttcaccatgttggccaggctggtgt
gaaactcctgaccttaagtgattcgcccactgtggcctcc
caaagtgctgggattacaggcgtgagctaccgcccccagc
ccctcccatcccacttctgtccagccccctagccctactt
tctttctgggatccaggagtccagatccccagccccctct
ccagattacattcatccaggcacaggaaaggacagggtca
ggaaaggaggactctgggcggcagcctccacattcccctt
ccacgcttggcccccagaatggaggagggtgtcttgatta
ctgggcgaggtgtcctcccttcctggggactgtggggggt
ggtcaaaagacctctatgccccacctccttcctccctctg
ccctgctgtgcctggggcagggggagaacagcccacctcg
tgactgggggctggcccagcccgccctatccctgggggag
ggggcgggacagggggagccctataattggacaagtctgg
gatccttgagtcctactcagccccagcggaggtgaaggac
gtccttccccaggagccggtgagaagcgcagtcgggggca
cggggatgagctcaggggcctctagaaagagctgggaccc
tgggaacccctggcctccaggtagtctcaggagagctact
cggggtcgggcttggggagaggaggagcgggggtgaggca
agcagcaggggactggacctgggaagggctgggcagcaga
gacgacccgacccgctagaaggtggggggggagagcagct
ggactgggatgtaagccatagcaggactccacgagttgtc
actatcatttatcgagcacctactgggtgtccccagtgtc
ctcagatctccataactggggagccaggggcagcgacacg
gtagctagccgtcgattggagaactttaaaatgaggactg
aattagctcataaatggaacacggcgcttaactgtgaggt
tggagcttagaatgtgaagggagaatgaggaatgcgagac
tgggactgagatggaaccggcggtggggagggggtggggg
gatggaatttgaaccccgggagaggaagatggaattttct
atggaggccgacctggggatggggagataagagaagacca
ggagggagttaaatagggaatgggttgggggcggcttggt
aaatgtgctgggattaggctgttgcagataatgcaacaag
gcttggaaggctaacctggggtgaggccgggttggggccg
ggctggggggggaggagtcctcactggcggttgattgaca
gtttctccttccccagactggccaatcacaggcaggaaga
tgaaggttctgtgggctgcgttgctggtcacattcctggc
aggtatgggggggggcttgctcggttccccccgctcctcc
ccctctcatcctcacctcaacctcctggccccattcaggc
agaccctgggccccctcttctgaggcttctgtgctgcttc
ctggctctgaacagcgatttgacgctctctgggcctcggt
ttcccccatccttgagataggagttagaagttgttttgtt
gttgttgtttgttgttgttgttttgtttttttgagatgaa
gtctcgctctgtcgcccaggctggagtgcagtggcgggat
ctcggctcactgcaagctccgcctcccaggtccacgccat
tctcctgcctcagcctcccaagtagctgggactacaggca
catgccaccacacccgactaacttttttgtattttcagta
gagacggggtttcaccatgttggccaggctggtctggaac
tcctgacctcaggtgatctgcccgtttcgatctcccaaag
tgctgggattacaggcgtgagccaccgcacctggctggga
gttagaggtttctaatgcattgcaggcagatagtgaatac
cagacacggggcagctgtgatctttattctccatcacccc
cacacagccctgcctggggcacacaaggacactcaataca
tgcttttccgctgggcgcggtggctcacccctgtaatccc
agcactttgggaggccaaggtgggaggatcacttgagccc
aggagttcaacaccagcctgggcaacatagtgagaccctg
tctctactaaaaatacaaaaattagccaggcatggtgcca
cacacctgtgctctcagctactcaggaggctgaggcagga
ggatcgcttgagcccagaaggtcaaggttgcagtgaacca
tgttcaggccgctgcactccagcctgggtgacagagcaag
accctgtttataaatacataatgctttccaagtgattaaa
ccgactcccccctcaccctgcccaccatggctccaaagaa
gcatttgtggagcaccttctgtgtgcccctaggtactaga
tgcctggacggggtcagaaggaccctgacccaccttgaac
ttgttccacacaggatgccaggccaaggtggagcaagcgg
tggagacagagccggagcccgagctgcgccagcagaccga
gtggcagagcggccagcgctgggaactggcactgggtcgc
ttttgggattacctgcgctgggtgcagacactgtctgagc
aggtgcaggaggagctgctcagctcccaggtcacccagga
actgaggtgagtgtccccatcctggcccttgaccctcctg
gtgggggctatacctccccaggtccaggtttcattctgcc
cctgtcgctaagtcttggggggcctgggtctctgctggtt
ctagcttcctcttcccatttctgactcctggctttagctc
tctggaattctctctctcagctttgtctctctctcttccc
ttctgactcagtctctcacactcgtcctggctctgtctct
gtccttccctagctcttttatatagagacagagagatggg
gtctcactgtgttgcccaggctggtcttgaacttctgggc
tcaagcgatcctcccgcctcggcctcccaaagtgctggga
ttagaggcatgagccaccttgcccggcctcctagctcctt
cttcgtctctgcctctgccctctgcatctgctctctgcat
ctgtctctgtctccttctctcggcctctgccccgttcctt
ctctccctcttgggtctctctggctcatccccatctcgcc
cgccccatcccagcccttctccccgcctcccactgtgcga
caccctcccgccctctcggccgcagggcgctgatggacga
gaccatgaaggagttgaaggcctacaaatcggaactggag
gaacaactgaccccggtggcggaggagacgcgggcacggc
tgtccaaggagctgcaggcggcgcaggcccggctgggcgc
ggacatggaggacgtgtgcggccgcctggtgcagtaccgc
ggcgaggtgcaggccatgctcggccagagcaccgaggagc
tgcgggtgcgcctcgcctcccacctgcgcaagctgcgtaa
gcggctcctccgcgatgccgatgacctgcagaagcgcctg
gcagtgtaccaggccggggcccgcgagggcgccgagcgcg
gcctcagcgagcaggcccagcagatacgcctgcaggccga
ggccttccaggcccgcctcaagagctggttcgagcccctg
gtggaagacatgcagcgccagtgggccgggctggtggaga
aggtgcaggctgccgtgggcaccagcgccgcccctgtgcc
cagcgacaatcactgaacgccgaagcctgcagccatgcga
ccccacgccaccccgtgcctcctgcctccgcgcagcctgc
agcgggagaccctgtccccgccccagccgtcctcctgggg
tggaccctagtttaataaagattcaccaagtttcacgcat
ctgctggcctccccctgtgatttcctctaagccccagcct
cagtttctctttctgcccacatactggccacacaattctc
agccccctcctctccatctgtgtctgtgtgtatctttctc
tctgcccttttttttttttttagacggagtctggctctgt
cacccaggctagagtgcagtggcacgatcttggctcactg
caacctctgcctcttgggttcaagcgattctgctgcctca
gtagctgggattacaggctcacaccaccacacccggctaa
tttttgtatttttagtagagacgagctttcaccatgttgg
ccaggcaggtctcaaactcctgaccaagtgatccacccgc
cggcctcccaaagtgctgagattacaggcctgagccacca
tgcccggcctctgcccctctttcttttttagggggcaggg
aaaggtctcaccctgtcacccgccatcacagctcactgca
gcctccacctcctggactcaagtgataagtgatcctcccg
cctcagcctttccagtagctgagactacaggcgcatacca
ctaggattaatttgggggggggggggtgtgtgtggagatg
gggtctggctttgttggccaggctgatgtggaattcctgg
gctcaagcgatactcccaccttggcctcctgagtagctga
gactactggctagcaccaccacacccagctttttattatt
atttgtagagacaaggtctcaatatgttgcccaggctagt
ctcaaacccctgggctcaagagatcctccgccatcggcct
cccaaagtgctgggattccaggcatggggctccgagcccg
gcctgcccaacttaataatacttgttcctcagagttgcaa
ctccaaatgacctgagattggtgcctttattctaagctat
tttcattttttttctgctgtcattattctcccccttctct
cctccagtcttatctgatatctgcctccttcccacccacc
ctgcaccccatcccacccctctgtctctccctgttctcct
caggagactctggcttcctgttttcctccacttctatctt
ttatctctccctcctacggtttcttttctttctccccggc
ctgcttgtttctcccccaacccccttcatctggatttctt
cttctgccattcagtttggtttgagctctctgcttctccg
gttccctctgagctagctgtcccttcacccactgtgaact
gggtttccctgcccaaccctcattctctttctttctttct
ttttttttttttttttttttttttttttttgagacagagt
cttgctctgttgcccagcctggagtgcagtggtgcaatct
tggttcactgcaacctccacttcccagattcaagcaattc
tcctgcctcagcctccagagtagctgggattacaggcgtg
tcccaccacacccgactaatttttgtatttttggtagaga
caaggcttcggcattgttggccaggcaggtctcgaactcc
tgacctcaagtaatctgcctgcctcaccctcccaaagtgc
tgggattacaggcatgagccacctcacccggaccatccct
cattctccatcctttcctccagttgtgatgtctacccctc
atgtttcccaacaagcctactgggtgctgaatccaggctg
ggaagagaagggagcggctcttctgtcggagtctgcacca
ggcccatgctgagacgagagctggcgctcagagaggggaa
gcttggatggaagcccaggagccgccggcactctcttctc
ctcccaccccctcagttctcagagacggggaggagggttc
ccaccaacgggggacaggctgagacttgagcttgtatctc
ctgggccagctgcaacatctgcttgtccctctgcccatct
tggctcctgcacaccctgaacttggtgctttccctggcac
tgctctgatcacccacgtggaggcagcacccctcccct.

In an aspect, a disclosed APOEe2 variant can comprise the sequence set forth below or a sequence having 70%, 75%, 80%, 85%, 90%, 95%, or more identity to the sequence set forth below:

(SEQ ID NO: 02)
Atgaaggttctgtgggctgcgttgctggtcacattcctgg
caggatgccaggccaaggtggagcaagcggtggagacaga
gccggagcccgagctgcgccagcagaccgagtggcagagc
ggccagcgctgggaactggcactgggtcgcttttgggatt
acctgcgctgggtgcagacactgtctgagcaggtgcagga
ggagctgctcagctcccaggtcacccaggaactgagggcg
ctgatggacgagaccatgaaggagttgaaggcctacaaat
cggaactggaggaacaactgaccccggtggcggaggagac
gcgggcacggctgtccaaggagctgcaggcggcgcaggcc
cggctgggcgcggacatggaggacgtgtgcggccgcctgg
tgcagtaccgcggcgaggtgcaggccatgctcggccagag
caccgaggagctgcgggtgcgcctcgcctcccacctgcgc
aagctgcgtaagcggctcctccgcgatgccgatgacctgc
agaagtgcctggcagtgtaccaggccggggcccgcgaggg
cgccgagcgcggcctcagcgccatccgcgagcgcctgggg
cccctggtggaacagggccgcgtgcgggccgccactgtgg
gctccctggccggccagccgctacaggagcgggcccaggc
ctggggcgagcggctgcgcgcgcggatggaggagatgggc
agccggacccgcgaccgcctggacgaggtgaaggagcagg
tggcggaggtgcgcgccaagctggaggagcaggcccagca
gatacgcctgcaggccgaggccttccaggcccgcctcaag
agctggttcgagcccctggtggaagacatgcagcgccagt
gggccgggctggtggagaaggtgcaggctgccgtgggcac
cagcgccgcccctgtgcccagcgacaatcactga.

In an aspect, a disclosed APOEe3 variant can comprise the sequence set forth below or a sequence having 70%, 75%, 80%, 85%, 90%, 95%, or more identity to the sequence set forth below:

(SEQ ID NO: 03)
Atgaaggttctgtgggctgcgttgctggtcacattcctgg
caggatgccaggccaaagtcgaacaggctgtcgaaactga
acccgaaccggagctgcgccagcagaccgagtggcagagc
ggccagcgctgggaactggcactgggtcgcttttgggatt
acctgcgctgggtgcagacactgtctgagcaggtgcagga
ggagctgctcagctcccaggtcacccaggaactgagggcg
ctgatggacgagaccatgaaggagttgaaggcctacaaat
cggaactggaggaacaactgaccccggtggcggaggagac
gcgggcacggctgtccaaggagctgcaggcggcgcaggcc
cggctgggcgcggacatggaggacgtgtgcggccgcctgg
tgcagtaccgcggcgaggtgcaggccatgctcggccagag
caccgaggagctgcgggtgcgcctcgcctcccacctgcgc
aagcttcgtaagcggctcctccgcgatgccgatgacctgc
agaagcgcctggcagtgtaccaggccggggcccgcgaggg
cgccgagcgcggcctcagcgccatccgcgagcgcctgggg
cccctggtggaacagggccgcgtgcgggccgccactgtgg
gctccctggccggccagccgctacaggagcgggcccaggc
ctggggcgagcggctgcgcgcgcggatggaggagatgggc
agccggacccgcgaccgcctggacgaggtgaaggagcagg
tggcggaggtgcgcgccaagctggaggagcaggcccagca
gatacgcctacaggccgaggccttccaggcccgcctcaag
agctggttcgagcccctggtggaagacatgcagcgccagt
gggccgggctggtggagaaggtgcaggctgccgtgggcac
cagcgccgcccctgtgcccagcgacaatcactga.

In an aspect, a disclosed APOEe4 variant can comprise the sequence set forth below or a sequence having 70%, 75%, 80%, 85%, 90%, 95%, or more identity to the sequence set forth

(SEQ ID NO: 04)
Atgaaggttctgtgggctgcgttgctggtcacattcctgg
caggatgccaggccaaagtcgaacaggctgtcgaaactga
acccgaaccggagctgcgccagcagaccgagtggcagagc
ggccagcgctgggaactggcactgggtcgcttttgggatt
acctgcgctgggtgcagacactgtctgagcaggtgcagga
ggagctgctcagctcccaggtcacccaggaactgagggcg
ctgatggacgagaccatgaaggagttgaaggcctacaaat
cggaactggaggaacaactgaccccggtggcggaggagac
gcgggcacggctgtccaaggagctgcaggcggcgcaggcc
cggctgggcgcggacatggaggacgtgcgcggccgcctgg
tgcagtaccgcggcgaggtgcaggccatgctcggccagag
caccgaggagctgcgggtgcgcctcgcctcccacctgcgc
aagcttcgtaagcggctcctccgcgatgccgatgacctgc
agaagcgcctggcagtgtaccaggccggggcccgcgaggg
cgccgagcgcggcctcagcgccatccgcgagcgcctgggg
cccctggtggaacagggccgcgtgcgggccgccactgtgg
gctccctggccggccagccgctacaggagcgggcccaggc
ctggggcgagcggctgcgcgcgcggatggaggagatgggc
agccggacccgcgaccgcctggacgaggtgaaggagcagg
tggcggaggtgcgcgccaagctggaggagcaggcccagca
gatacgcctacaggccgaggccttccaggcccgcctcaag
agctggttcgagcccctggtggaagacatgcagcgccagt
gggccgggctggtggagaaggtgcaggctgccgtgggcac
cagcgccgcccctgtgcccagcgacaatcactga.

1. An isolated nucleic acid molecule, comprising:
a nucleic acid sequence encoding
(i) a Cas endonuclease,
(ii) at least one polypeptide having repressor activity, and
(iii) at least one guide RNA designed to target (i) exon 4 of the APOE gene, (ii) the promoter region of the APOE gene, or (iii) a regulatory element within the APOE gene.

2. The isolated nucleic acid molecule of claim 1, wherein the Cas endonuclease comprises Cas9 or VRER Cas9.

3. The isolated nucleic acid molecule of claim 1, wherein the at least one encoded polypeptide having repressor activity comprises Krüppel-associated box (KRAB), the transcription repression domain (TRD) of Methyl-CpG Binding Protein 2 (MeCP2), or a fusion of KRAB-MeCP2 (KRAB-MeCP2).

4. The isolated nucleic acid molecule of claim 1, wherein, in exon 4 of the APOE gene, the at least one gRNA is designed to target a protospacer-adjacent motif (PAM) created by a SNP rs429358 in exon 4 of the APOE gene.

5. The isolated nucleic acid molecule of claim 1, where the Cas endonuclease comprises Cas9 VRER and the polypeptide having repressor activity comprises Krüppel-associated box (KRAB), the transcription repression domain (TRD) of Methyl-CpG Binding Protein 2 (MeCP2), or a fusion of KRAB-MeCP2 (KRAB-MeCP2).

6. A viral vector comprising the isolated nucleic acid molecule of claim 1.

7. The viral vector of claim 6, wherein the viral vector comprises one or more promoters operably linked to the isolated nucleic acid molecule, and wherein the one or more promoters drive the expression of the at least one gRNA and the Cas9 endonuclease.

8. (canceled)

9. The viral vector of claim 6, wherein the viral vector comprises one or more regulatory elements.

10. The viral vector of claim 9, wherein the one or more regulatory elements comprise a Sp1 responsive element, a p2A signal, a woodchuck hepatitis virus post-transcriptional regulatory element, a Phi signal-packaging signal, a rev responsive element, a 5′-LTR, and a 3′-LTR.

11. (canceled)

12. The viral vector of claim 6, wherein the viral vector is a lentiviral vector.

13.-24. (canceled)

25. A method of administering precision gene therapy to a subject, the method comprising:
administering to a human subject in need thereof a therapeutically effective amount of the viral vector of claim 6, wherein the expression of APOE is reduced or wherein the expression of the APOE e4 allele is reduced.

26. (canceled)

27. The method of claim 25, wherein the human subject is at risk of developing, suspected of having, or has been diagnosed with having Alzheimer's disease.

28. The method of claim 25, wherein the pathological phenotype associated with Alzheimer's disease is reduced.

29. The method of claim 25, wherein administering the viral vector to the subject comprises intravenous administration, intracerebral administration, intra-CSF administration, intracerebroventricular (ICV) administration, intraventricular administration, intra-cisterna magna (ICM) administration, intraparenchymal administration, intrathecal administration, or any combination thereof.

30. The method of claim 25, further comprising administering to the human subject a therapeutically effective amount of a therapeutic agent and/or an immune modulator.

31.-36. (canceled)

37. The isolated nucleic acid molecule of claim 1, wherein the at least one encoded polypeptide comprises the sequence set forth in any one of SEQ ID NO:57, SEQ ID NO:58, SEQ ID NO:62, or SEQ ID NO:63.

38. The isolated nucleic acid molecule of claim 1, wherein the at least one gRNA designed to target exon 4 of the APOE gene comprises the sequence set forth in any one of SEQ ID NO:05-SEQ ID NO:14.

39. The isolated nucleic acid molecule of claim 2, wherein the Cas9 comprises a sequence having at least 75%, at least 80%, at least 85%, at least 90%, or at least 95% identity to the sequence set forth in SEQ ID NO:64 or SEQ ID NO:65, or wherein the VRER Cas9 comprises a sequence having at least 75%, at least 80%, at least 85%, at least 90%, or at least 95% identity to the sequence set forth in SEQ ID NO:15.

40. The method of claim 25, wherein the at least one gRNA is designed to target the promoter region of the APOE gene or a regulatory element within the APOE gene, and wherein the expression of APOE is reduced.

41. The method of claim 25, wherein the at least one gRNA is designed to target a protospacer-adjacent motif (PAM) created by a SNP rs429358 in exon 4 of the APOE gene, and wherein the expression of the APOE e4 allele is reduced.


SEQUENCE LISTING (65 sequences)

SEQ ID NO: 1 - DNA, 6443 bp, Artificial Sequence
  Synthetic - ApoE gene
ggaacttgatgctcagagaggacaagtcatttgcccaaggtcacacagctggcaactggcagagccaggattcacgccctggcaatttgactccagaatcctaaccttaacccagaagcacggcttcaagcccctggaaaccacaatacctgtggcagccagggggaggtgctggaatctcatttcacatgtggggagggggctcccctgtgctcaaggtcacaaccaaagaggaagctgtgattaaaacccaggtcccatttgcaaagcctcgacttttagcaggtgcatcatactgttcccacccctcccatcccacttctgtccagccgcctagccccactttcttttttttctttttttgagacagtctccctcttgctgaggctggagtgcagtggcgagatctcggctcactgtaacctccgcctcccgggttcaagcgattctcctgcctcagcctcccaagtagctaggattacaggcgcccgccaccacgcctggctaacttttgtatttttagtagagatggggtttcaccatgttggccaggctggtgtgaaactcctgaccttaagtgattcgcccactgtggcctcccaaagtgctgggattacaggcgtgagctaccgcccccagcccctcccatcccacttctgtccagccccctagccctactttctttctgggatccaggagtccagatccccagccccctctccagattacattcatccaggcacaggaaaggacagggtcaggaaaggaggactctgggcggcagcctccacattccccttccacgcttggcccccagaatggaggagggtgtcttgattactgggcgaggtgtcctcccttcctggggactgtggggggtggtcaaaagacctctatgccccacctccttcctccctctgccctgctgtgcctggggcagggggagaacagcccacctcgtgactgggggctggcccagcccgccctatccctgggggagggggcgggacagggggagccctataattggacaagtctgggatccttgagtcctactcagccccagcggaggtgaaggacgtccttccccaggagccggtgagaagcgcagtcgggggcacggggatgagctcaggggcctctagaaagagctgggaccctgggaacccctggcctccaggtagtctcaggagagctactcggggtcgggcttggggagaggaggagcgggggtgaggcaagcagcaggggactggacctgggaagggctgggcagcagagacgacccgacccgctagaaggtggggtggggagagcagctggactgggatgtaagccatagcaggactccacgagttgtcactatcatttatcgagcacctactgggtgtccccagtgtcctcagatctccataactggggagccaggggcagcgacacggtagctagccgtcgattggagaactttaaaatgaggactgaattagctcataaatggaacacggcgcttaactgtgaggttggagcttagaatgtgaagggagaatgaggaatgcgagactgggactgagatggaaccggcggtggggagggggtggggggatggaatttgaaccccgggagaggaagatggaattttctatggaggccgacctggggatggggagataagagaagaccaggagggagttaaatagggaatgggttgggggcggcttggtaaatgtgctgggattaggctgttgcagataatgcaacaaggcttggaaggctaacctggggtgaggccgggttggggccgggctgggggtgggaggagtcctcactggcggttgattgacagtttctccttccccagactggccaatcacaggcaggaagatgaaggttctgtgggctgcgttgctggtcacattcctggcaggtatgggggcggggcttgctcggttccccccgctcctccccctctcatcctcacctcaacctcctggccccattcaggcagaccctgggccccctcttctgaggcttctgtgctgcttcctggctctgaacagcgatttgacgctctctgggcctcggtttcccccatccttgagataggagttagaagttgttttgttgttgttgtttgttgttgttgttttgtttttttgagatgaagtctcgctctgtcgcccaggctggagtgcagtggcgggatctcggctcactgcaagctccgcctcccaggtccacgccattctcctgcctcagcctcccaagtagctgggactacaggcacatgccaccacacccgactaacttttttgtattttcagtagagacggggtttcaccatgttggccaggctggtctggaactcctgacctcaggtgatctgcccgtttcgatctcccaaagtgctgggattacaggcgtgagccaccgcacctggctgggagttagaggtttctaatgcattgcaggcagatagtgaataccagacacggggcagctgtgatctttattctccatcacccccacacagccctgcctggggcacacaaggacactcaatacatgcttttccgctgggcgcggtggctcacccctgtaatcccagcactttgggaggccaaggtgggaggatcacttgagcccaggagttcaacaccagcctgggcaacatagtgagaccctgtctctactaaaaatacaaaaattagccaggcatggtgccacacacctgtgctctcagctactcaggaggctgaggcaggaggatcgcttgagcccagaaggtcaaggttgcagtgaaccatgttcaggccgctgcactccagcctgggtgacagagcaagaccctgtttataaatacataatgctttccaagtgattaaaccgactcccccctcaccctgcccaccatggctccaaagaagcatttgtggagcaccttctgtgtgcccctaggtactagatgcctggacggggtcagaaggaccctgacccaccttgaacttgttccacacaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgaggtgagtgtccccatcctggcccttgaccctcctggtgggcggctatacctccccaggtccaggtttcattctgcccctgtcgctaagtcttggggggcctgggtctctgctggttctagcttcctcttcccatttctgactcctggctttagctctctggaattctctctctcagctttgtctctctctcttcccttctgactcagtctctcacactcgtcctggctctgtctctgtccttccctagctcttttatatagagacagagagatggggtctcactgtgttgcccaggctggtcttgaacttctgggctcaagcgatcctcccgcctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatctgctggcctccccctgtgatttcctctaagccccagcctcagtttctctttctgcccacatactggccacacaattctcagccccctcctctccatctgtgtctgtgtgtatctttctctctgcccttttttttttttttagacggagtctggctctgtcacccaggctagagtgcagtggcacgatcttggctcactgcaacctctgcctcttgggttcaagcgattctgctgcctcagtagctgggattacaggctcacaccaccacacccggctaatttttgtatttttagtagagacgagctttcaccatgttggccaggcaggtctcaaactcctgaccaagtgatccacccgccggcctcccaaagtgctgagattacaggcctgagccaccatgcccggcctctgcccctctttcttttttagggggcagggaaaggtctcaccctgtcacccgccatcacagctcactgcagcctccacctcctggactcaagtgataagtgatcctcccgcctcagcctttccagtagctgagactacaggcgcataccactaggattaatttgggggggggggtggtgtgtgtggagatggggtctggctttgttggccaggctgatgtggaattcctgggctcaagcgatactcccaccttggcctcctgagtagctgagactactggctagcaccaccacacccagctttttattattatttgtagagacaaggtctcaatatgttgcccaggctagtctcaaacccctgggctcaagagatcctccgccatcggcctcccaaagtgctgggattccaggcatggggctccgagcccggcctgcccaacttaataatacttgttcctcagagttgcaactccaaatgacctgagattggtgcctttattctaagctattttcattttttttctgctgtcattattctcccccttctctcctccagtcttatctgatatctgcctccttcccacccaccctgcaccccatcccacccctctgtctctccctgttctcctcaggagactctggcttcctgttttcctccacttctatcttttatctctccctcctacggtttcttttctttctccccggcctgcttgtttctcccccaacccccttcatctggatttcttcttctgccattcagtttggtttgagctctctgcttctccggttccctctgagctagctgtcccttcacccactgtgaactgggtttccctgcccaaccctcattctctttctttctttctttttttttttttttttttttttttttttttgagacagagtcttgctctgttgcccagcctggagtgcagtggtgcaatcttggttcactgcaacctccacttcccagattcaagcaattctcctgcctcagcctccagagtagctgggattacaggcgtgtcccaccacacccgactaatttttgtatttttggtagagacaaggcttcggcattgttggccaggcaggtctcgaactcctgacctcaagtaatctgcctgcctcaccctcccaaagtgctgggattacaggcatgagccacctcacccggaccatccctcattctccatcctttcctccagttgtgatgtctacccctcatgtttcccaacaagcctactgggtgctgaatccaggctgggaagagaagggagcggctcttctgtcggagtctgcaccaggcccatgctgagacgagagctggcgctcagagaggggaagcttggatggaagcccaggagccgccggcactctcttctcctcccaccccctcagttctcagagacggggaggagggttcccaccaacgggggacaggctgagacttgagcttgtatctcctgggccagctgcaacatctgcttgtccctctgcccatcttggctcctgcacaccctgaacttggtgctttccctggcactgctctgatcacccacgtggaggcagcacccctcccct

SEQ ID NO: 2 - DNA, 954 bp, Artificial Sequence
  Synthetic - APOEe2
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 3 - DNA, 954 bp, Artificial Sequence
  Synthetic - APOEe3
atgaaggttctgtgggctgcgttgctggtcacattcctggcaggatgccaggccaaagtcgaacaggctgtcgaaactgaacccgaaccggagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagcttcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctacaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 4 - DNA, 954 bp, Artificial Sequence
  APOEe4
atgaaggttctgtgggctgcgttgctggtcacattcctggcaggatgccaggccaaagtcgaacaggctgtcgaaactgaacccgaaccggagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgcgcggccgcctggtgcagtaccgcggcgaggtgcaggccatgctcggccagagcaccgaggagctgcgggtgcgcctcgcctcccacctgcgcaagcttcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctacaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 5 - DNA, 20 bp, Artificial Sequence
  gRNA1 targeting promoter region of ApoE using SpCas9
gacagggggagccctataat

SEQ ID NO: 6 - DNA, 20 bp, Artificial Sequence
  gRNA2 targeting promoter region of ApoE using SpCas9
tcaggagagctactcggggt

SEQ ID NO: 7 - DNA, 20 bp, Artificial Sequence
  gRNA3 targeting promoter region of ApoE using SpCas9
actgggatgtaagccatagc

SEQ ID NO: 8 - DNA, 20 bp, Artificial Sequence
  gRNA4 targeting promoter region of ApoE using SpCas9
gttggagcttagaatgtgaa

SEQ ID NO: 9 - DNA, 20 bp, Artificial Sequence
  Synthetic - gRNA1 targeting promoter region of ApoE using VRER Cas9
gccctatccctgggggaggg

SEQ ID NO: 10 - DNA, 20 bp, Artificial Sequence
  Synthetic - gRNA2 targeting promoter region of ApoE using VRER Cas9
tcgggcttggggagaggagg

SEQ ID NO: 11 - DNA, 20 bp, Artificial Sequence
  Synthetic - gRNA3 targeting promoter region of ApoE using VRER Cas9
ctctccccaccccaccttct

SEQ ID NO: 12 - DNA, 20 bp, Artificial Sequence
  Synthetic - gRNA4 targeting promoter region of ApoE using VRER Cas9
tgtgaagggagaatgaggaa

SEQ ID NO: 13 - DNA, 19 bp, Artificial Sequence
  gRNA targeting GFP ORF using SpCas9
ggcgaggagctgttcaccg

SEQ ID NO: 14 - DNA, 20 bp, Artificial Sequence
  Synthetic - gRNA targeting GFP ORF using VRER- SpCas9
gccacaagttcagcgtgtcc

SEQ ID NO: 15 - DNA, 4107 bp, Artificial Sequence
  Synthetic - VRER
atggataaaaagtattctattggtttagacatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgactga

SEQ ID NO: 16 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 17 - protein, 311 aa, Artificial Sequence
  Synthetic - DNMT3A
PSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 18 - protein, 933 aa, Artificial Sequence
  Synthetic - DNMT3A
CCCTCCCGGCTCCAGATGTTCTTCGCTAATAACCACGACCAGGAATTTGACCCTCCAAAGGTTTACCCACCTGTCCCAGCTGAGAAGAGGAAGCCCATCCGGGTGCTGTCTCTCTTTGATGGAATCGCTACAGGGCTCCTGGTGCTGAAGGACTTGGGCATTCAGGTGGACCGCTACATTGCCTCGGAGGTGTGTGAGGACTCCATCACGGTGGGCATGGTGCGGCACCAGGGGAAGATCATGTACGTCGGGGACGTCCGCAGCGTCACACAGAAGCATATCCAGGAGTGGGGCCCATTCGATCTGGTGATTGGGGGCAGTCCCTGCAATGACCTCTCCATCGTCAACCCTGCTCGCAAGGGCCTCTACGAGGGCACTGGCCGGCTCTTCTTTGAGTTCTACCGCCTCCTGCATGATGCGCGGCCCAAGGAGGGAGATGATCGCCCCTTCTTCTGGCTCTTTGAGAATGTGGTGGCCATGGGCGTTAGTGACAAGAGGGACATCTCGCGATTTCTCGAGTCCAACCCTGTGATGATTGATGCCAAAGAAGTGTCAGCTGCACACAGGGCCCGCTACTTCTGGGGTAACCTTCCCGGTATGAACAGGCCGTTGGCATCCACTGTGAATGATAAGCTGGAGCTGCAGGAGTGTCTGGAGCATGGCAGGATAGCCAAGTTCAGCAAAGTGAGGACCATTACTACGAGGTCAAACTCCATAAAGCAGGGCAAAGACCAGCATTTTCCTGTGTTCATGAATGAGAAAGAGGACATCTTATGGTGCACTGAAATGGAAAGGGTATTTGGTTTCCCAGTCCACTATACTGACGTGTCCAACATGAGCCGCTTGGCGAGGCAGAGACTGCTGGGCCGGTCATGGAGCGTGCCAGTCATCCGCCACCTCTTCGCTCCGCTGAAGGAGTATTTTGCGTGTGTG

SEQ ID NO: 19 - protein, 1702 aa, Artificial Sequence
  Synthetic - dCas9-DNMT3A fusion protein
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKLEGGGGSGSPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFAC

SEQ ID NO: 20 - DNA, 5109 bp, Artificial Sequence
  Synthetic - dCas9-DNMT3A fusion protein
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtg

SEQ ID NO: 21 - DNA, 14092 bp, Artificial Sequence
  Synthetic - pBK546 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgtgtacacgtctctgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 22 - DNA, 13812 bp, Artificial Sequence
  Synthetic - pBK539 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgtgtacacgtctctgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggggccggcccggatccggcgcaacaaacttctctctgctgaaacaagccggagatgtcgaagagaatcctggaccgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtgccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 23 - DNA, 13039 bp, Artificial Sequence
  Synthetic - pBK500 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgctcagggtagatagctggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 24 - DNA, 13813 bp, Artificial Sequence
  pBK744 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtttttcaagcggaaacgctagttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggggccggcccggatccggcgcaacaaacttctctctgctgaaacaagccggagatgtcgaagagaatcctggaccgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtgccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 25 - DNA, 20 bp, Artificial Sequence
  gRNA1 cloned into pBK1026 and pBK1030
gacagggggagccctataat

SEQ ID NO: 26 - DNA, 20 bp, Artificial Sequence
  Synthetic - gRNA2 cloned into pBK1027 and pBK1031
tcaggagagctactcggggt

SEQ ID NO: 27 - DNA, 20 bp, Artificial Sequence
  Synthetic - gRNA3 cloned into pBK1028 and pBK1032
actgggatgtaagccatagc

SEQ ID NO: 28 - DNA, 20 bp, Artificial Sequence
  Synthetic - gRNA4 cloned into pBK1029 and pBK1033
gttggagcttagaatgtgaa

SEQ ID NO: 29 - DNA, 14093 bp, Artificial Sequence
  Synthetic - pBK1026 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggacagggggagccctataatgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 30 - DNA, 14093 bp, Artificial Sequence
  Synthetic - pBK1027 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcaggagagctactcggggtgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 31 - DNA, 14093 bp, Artificial Sequence
  Synthetic - pBK1028 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactgggatgtaagccatagcgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 32 - DNA, 14093 bp, Artificial Sequence
  Synthetic - pBK1029plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttggagcttagaatgtgaagttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 33 - DNA, 14093 bp, Artificial Sequence
  pBK1030 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggacagggggagccctataatgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgcgaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 34 - DNA, 14093 bp, Artificial Sequence
  Synthetic - pBK1031 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcaggagagctactcggggtgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgcgaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 35 - DNA, 14093 bp, Artificial Sequence
  Synthetic - pBK1032 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactgggatgtaagccatagcgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgcgaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 36 - DNA, 14093 bp, Artificial Sequence
  Synthetic - pBK1033 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttggagcttagaatgtgaagttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgcgaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 37 - DNA, 5208 bp, Artificial Sequence
  Synthetic - SpCas9 VRER sequence linked with DNMT3A
atggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatcccccaagaagaagaggaaagtcctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtg

SEQ ID NO: 38 - protein, 1688 aa, Artificial Sequence
  VRER- DNMT3A
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLAGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNLKGSPEDNEQKQLFVEQHKHYLDEIIEQISEFSKRVILADANLDKVLSAYNKHRDKPIREQAENIIHLFTLTNLGAPAAFKYFDTTIDRKEYRSTKEVLDATLIHQSITGLYETRIDLSQLGGDGSPKKKVLEGGGGSGSPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 39 - DNA, 20 bp, Artificial Sequence
  Synthetic - gRNA1 cloned into pBK1105 and pBK1109 plasmids
gccctatccctgggggaggg

SEQ ID NO: 40 - DNA, 20 bp, Artificial Sequence
  gRNA2 cloned into pBK1106 and pBK1110 plasmids
tcgggcttggggagaggagg

SEQ ID NO: 41 - DNA, 20 bp, Artificial Sequence
  Synthetic - gRNA3 cloned into pBK1107 and pBK1111 plasmids
ctctccccaccccaccttct

SEQ ID NO: 42 - DNA, 20 bp, Artificial Sequence
  Synthetic - gRNA4 cloned into pBK1108 and pBK1112 plasmids
tgtgaagggagaatgaggaa

SEQ ID NO: 43 - DNA, 13330 bp, Artificial Sequence
  Synthetic - pBK1105 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggccctatccctgggggaggggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatcccccaagaagaagaggaaagtcctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 44 - DNA, 13330 bp, Artificial Sequence
  pBK1106 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcgggcttggggagaggagggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatcccccaagaagaagaggaaagtcctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 45 - DNA, 13330 bp, Artificial Sequence
  pBK1107 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctctccccaccccaccttctgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatcccccaagaagaagaggaaagtcctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 46 - DNA, 13330 bp, Artificial Sequence
  Synthetic - pBK1108 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgtgaagggagaatgaggaagttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatcccccaagaagaagaggaaagtcctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 47 - DNA, 13330 bp, Artificial Sequence
  pBK1109 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggccctatccctgggggaggggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatcccccaagaagaagaggaaagtcctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgcgaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 48 - DNA, 13330 bp, Artificial Sequence
  Synthetic - pBK1110 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcgggcttggggagaggagggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatcccccaagaagaagaggaaagtcctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgcgaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 49 - DNA, 13330 bp, Artificial Sequence
  Synthetic - pBK1111 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctctccccaccccaccttctgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatcccccaagaagaagaggaaagtcctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgcgaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 50 - DNA, 13330 bp, Artificial Sequence
  Synthetic - pBK1112 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgtgaagggagaatgaggaagttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatcccccaagaagaagaggaaagtcctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgcgaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 51 - DNA, 20 bp, Artificial Sequence
  Synthetic - gRNA1 cloned into pBK1426 and pBK1428 plasmids
gcggtactgcaccaggcggc

SEQ ID NO: 52 - DNA, 20 bp, Artificial Sequence
  Synthetic - gRNA2 cloned into pBK1427 and pBK1429 plasmids
gggcgcggacatggaggacg

SEQ ID NO: 53 - DNA, 13330 bp, Artificial Sequence
  Synthetic - pBK1426 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcggtactgcaccaggcggcgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatcccccaagaagaagaggaaagtcctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 54 - DNA, 13330 bp, Artificial Sequence
  Synthetic - pBK1427 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggcgcggacatggaggacggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatcccccaagaagaagaggaaagtcctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 55 - DNA, 13327 bp, Artificial Sequence
  Synthetic - pBK1428 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcggtactgcaccaggcggcgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatcccccaagaagaagaggaaagtcctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgcgaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacct

SEQ ID NO: 56 - DNA, 13330 bp, Artificial Sequence
  Synthetic - pBK1429 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggcgcggacatggaggacggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatcccccaagaagaagaggaaagtcctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgcgaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 57 - DNA, 327 bp, Artificial Sequence
  Synthetic - MeCP2 Transcription Repression Domain (TRD)
atgtcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggagtaa

SEQ ID NO: 58 - protein, 108 aa, Artificial Sequence
  Synthetic - MeCP2 Transcription Repression Domain (TRD)
MSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRE

SEQ ID NO: 59 - DNA, 12952 bp, Artificial Sequence
  Synthetic - pBK1531 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcggtactgcaccaggcggcgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatccccaaagaagaaacggaaggtgggtggaggaagtggcgggtcaggtggctctagacggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctcgggaggtggttcgggaggtggttcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggagggcgcgcccaagaagaagaggaaagtctccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 60 - DNA, 12952 bp, Artificial Sequence
  Synthetic - pBK1532 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggcgcggacatggaggacggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatccccaaagaagaaacggaaggtgggtggaggaagtggcgggtcaggtggctctagacggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctcgggaggtggttcgggaggtggttcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggagggcgcgcccaagaagaagaggaaagtctccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 61 - DNA, 12951 bp, Artificial Sequence
  Synthetic - pBK1536 plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgtgtacacgtctctgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatccccaaagaagaaacggaaggtgggtggaggaagtggcgggtcaggtggctctagacggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctcgggaggtggttcgggaggtggttcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggagggcgcgcccaagaagaagaggaaagtctccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 62 - DNA, 537 bp, Artificial Sequence
  Synthetic - KRAB-MeCP2 Repressor
atgcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctcgggaggtggttcgggaggtggttcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggagtaa

SEQ ID NO: 63 - protein, 178 aa, Artificial Sequence
  Synthetic - KRAB-MeCP2 Repressor Protein ORF
MRTLVTFKDVFVDFTREEWKLLDTAQQIVYRNVMLENYKNLVSLGYQLTKPDVILRLEKGEEPSGGGSGGGSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRE

SEQ ID NO: 64 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 65 - protein, 1368 aa, Streptococcus pyogenes
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRLNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGLTIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD